(19) United States
(12) Patent Application Publication
(10) Pub. No.: US 2010/0004159 A1
(43) Pub. Date: Jan. 7, 2010

(54) SUBSTITUTED IMIDAZOLONE DERIVATIVES, PREPARATIONS AND USES

(75) Inventors: Edith Bouey, Lorgies (FR); Christophe Masson, Lambersart (FR); Karine Bertrand, Frelinghien (FR)

Correspondence Address: NIXON & VANDERHYE, PC, 901 NORTH GLEBE ROAD, 11TH FLOOR, ARLINGTON, VA, 22203, US

(73) Assignee: GENFIT, Loos (FR)

(21) Appl. No.: 12/309,642

(22) PCT Filed: Jul. 24, 2007
(86) PCT No.: PCT/FR2007/051716

§ 371 Date: Jan. 26, 2009

(30) Foreign Application Priority Data
Jul. 24, 2006 (FR) 0606752

(51) Int. Cl. A61K 31/4166; C07D 235/26; C07D 233/70; C07D 513/04; C07D 403/12; A61K 31/429; A61K 38/28; A61P 3/00; A61P 9/00; A61P 29/00; A61P 25/00

(52) U.S. Cl. 514/4; 548/300.7; 548/325.5; 548/147; 548/253; 514/381; 514/368; 514/386

(57) ABSTRACT

The present invention relates to polysubstituted imidazolone derivatives, to the pharmaceutical compositions comprising them and to the therapeutic uses thereof in the human and animal health fields. The present invention also relates to a process for preparing these derivatives.

The present invention relates to polysubstituted imidazolone derivatives, pharmaceutical compositions comprising them and the therapeutic uses thereof, in particular in the human and animal health fields. The present invention also relates to a process for preparing these derivatives.

The inventors unexpectedly discovered a family of original molecules that have a “multimodal” action mechanism. The compounds according to the invention present PPAR (Peroxisome Proliferator-Activated Receptor) activating properties, notably PPARα, and angiotensin II AT1receptor antagonist properties. The molecules described in the invention are therefore of particular interest for the treatment of pathologies linked to lipid and glucid metabolism disorders and/or hypertension.

The compounds according to the invention, because of their PPAR agonist properties, are of particular interest for the treatment of pathologies related to deregulations in lipid and/or glucid metabolism, such as diabetes, obesity, dyslipidemias, or inflammation, as well as for reducing the global cardiovascular risks. PPARs (α, γ and δ) are known to be involved in such pathologies (Kota B P et al., 2005): ligands of their receptors, for example fibrates or thiazolidinediones, are therefore marketed for the treatment of these pathologies (Lefebvre P et al., 2006) and various PPAR modulators, agonist or antagonist, selective or non-selective, are currently in high development for the treatment of these pathologies. The family of PPARs includes three distinct members, known as α, β, and δ (also known as β), each being coded by a different gene. These receptors belong to the nuclear receptor and transcription factor superfamily which are activated upon contact with certain fatty acids and/or their lipid metabolites.

Additionally, compounds according to the invention are linked to the angiotensin II AT1 receptor. Angiotensin II, an octapeptide produced by the renin-angiotensin system (RAS), is a powerful vasoconstrictor. Angiotensin II comes from the cleavage of angiotensin I by angiotensin converting enzyme (ACE). Angiotensin II produces its effects by stimulating specific receptors called AT1 and AT2 (de Gasparo M et al., 2000). AT1 receptor has a ubiquitous distribution and is involved in the main physiological actions of angiotensin II: the activation of the AT1 receptor stimulates vasoconstriction, growth, and cellular proliferation by activating different tyrosine kinases.

The present invention therefore relates to new compounds in which the PPAR/AT1 “multimodal” action mechanism permits greater therapeutic progress. Diabetes, obesity, dyslipidemias (elevated plasma levels of LDL (low density lipoproteins), cholesterol and triglycerides, low HDL cholesterol (high density lipoproteins), etc.), and hypertension are clearly-identified cardiovascular risk factors (Mensah M, 2004), which predispose an individual to develop a cardiovascular pathology.

The prevalence of these risk factors is rampantly increasing: the prevalence of dyslipidemias, whose treatment mainly uses statins, fibrates, and other triglyceride reducers, affected 43.6% of the population in 2004 in the principal developed countries and hypertension affected 30.1 % (Fox-Tucker J, 2005). Hypertension, characterized by elevated arterial pressure (greater than 140/90 mm Hg), is currently treated using 6 types of molecules: diuretics, beta blockers, angiotensin conversion enzyme inhibitors, calcium inhibitors, vasodilators, or alpha-blockers.

Additionally, the lifestyle risk factors such as tobacco consumption, a sedentary lifestyle, and an unbalanced diet, should be also considered. These factors have a synergetic effect: the simultaneous presence of several of these factors dramatically increases cardiovascular risks. It is therefore appropriate to speak in terms of global risk for cardiovascular diseases.

According to the International Atherosclerosis Society (International Atherosclerosis Society, 2003), cardiovascular disease is the primary cause of death in industrialized countries and is becoming ever more prevalent in developing countries. The principal cardiovascular diseases are heart disease, cerebral ischemia, and peripheral arterial disease.

These data therefore justify taking vigorous measures to significantly reduce cardiovascular morbidity and mortality rates and reveal the necessity of finding effective treatments, in conjunction with life style modification. Taking into account the risk factors for cardiovascular diseases and their consequences, this is a worldwide emergency.

Current therapeutic strategies consist in, on one hand, combining several medications in order to reduce different individual risk factors (Morphy R and Rankovic Z, 2005), which can sometimes provoke serious side effects (for example, simultaneously administering fibrates and statins increases risk of myopathy (Denke Mass., 2003)), and, on the other hand, developing medications whose the “multimodal” effect presents advantages linked to the administration of just one active constituent, in terms of compliance, tolerance, pharmacokinetics, and pharmacodynamics. This type of product may reduce the risk of cardiovascular disease and allows the treatment of each dysfunction and its consequences, individually considered (dyslipidemias, diabetes, etc.).

The combination of PPAR agonist molecules and angiotensin II receptor antagonists has been the subject of different publications. A recent clinical study has shown that the combination of fenofibrate and candesartan improves endothelial function and reduces inflammation markers more completely in hypertensive hypertriglyceridemic patients (Koh K K et al., 2006). Fenofibrate also seems to prevent the development of angiotensin II-induced hypertension in mice (Vera T et al., 2005). Patent application WO 2004/017896 describes the combination of a PPARα/γ agonist and an AT1 angiotensin receptor antagonist useful for the treatment of diabetes, metabolic syndrome etc.

Benson et al. (Benson S C et al., 2004) also mentions the advantages of molecules having both angiotensin II antagonist properties and PPARγ agonist properties, for the treatment of metabolic syndrome. It was recently shown that angiotensin II antagonists selectively activate PPARγ (Benson S C, Pershadsingh H A, Ho Cl, Chittiboyina A, Desai P, Pravenec M, Qi N, Wang J, Avery M A and Kurtz T W, 2004, Kurtz T W, 2005). This effect is specific to PPARγ, no activation of PPARα or PPARδ has been shown. Thiazolidinediones (PPARγ agonists) also seem to regulate the signal of angiotensin on multiple levels, by significantly reducing the expression of the AT1 receptor and by blocking the transduction of the signal via this receptor to suppress the vascular remodelling, the formation of the atherosclerotic lesion, and oxidative stress (Kintscher U et al., 2004). The patent applications WO 2004/060399 and WO 2004/014308 describe compounds with PPAR agonist and angiotensin II receptor antagonist properties, which is of interest for weight loss, and the treatment of cardiovascular diseases and insulin-resistance syndromes.

The molecules described in the invention, thanks to their PPAR agonist/AT1 antagonist action, are of particular interest for the treatment of pathologies linked to lipid and glucid disorders and/or hypertension such as complications associated with metabolic syndrome, diabetes, dyslipidemias, atherosclerosis, cardiovascular diseases, obesity, hypertension, inflammatory diseases (asthma, etc.), insulin resistance, neurodegenerative pathologies, cancers, etc., as well as for reducing the global cardiovascular risk. Compounds according to the invention are especially of interest for the treatment of dyslipidemias and/or hypertension (especially hypertension associated or not with dyslipidemias and/or hypertension associated or not with diabetes).

These goals as well as other ones were reached by the present invention which relates to polysubstituted derivatives of imidazolones according to general formula (I):

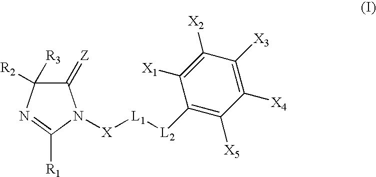

in which:

R1 represents a hydrogen atom or an alkyl, cycloalkyl, alkyloxy, alkylthio, alkenyl, alkynyl, aryl, arylalkyl, heteroaryl group or a heterocycle;

R2 and R3, identical or different, represent independently a hydrogen atom or an alkyl, alkenyl, alkynyl, cycloalkyl, aryl, arylalkyl group or a heterocycle, or R2 and R3 together may form, with the carbon they are linked to, a cycle or a heterocycle;

Z represents an oxygen or a sulfur atom;

X represents an alkyl group whose principal chain has from 1 to 6 carbon atoms or X represents an alkenyl or alkynyl group whose principal chain has from 2 to 6 carbon atoms;

L1 represents:
(i) a covalent bond, or
(ii) a heterocycle, or
(iii) a formula (II) group defined as follows:

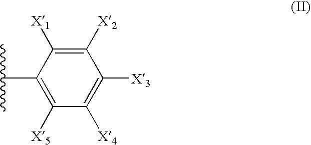

X′1, X′2, X′3, X′4, and X′5, identical or different, independently representing a hydrogen or halogen atom, an NO₂, nitrile, alkyl, cycloalkyl, alkenyl, alkynyl, aryl, arylalkyl, —OR4, —SR4, —NR4R5, —SOR6, or —SO₂R6 group, or a heterocycle, in which one of X′1, X′2, X′3, X′4, and X′5 is L2;

L2 represents:
(i) a covalent bond, or
(ii) a carbonyl group (CO), or
(iii) an oxygen or sulfur atom, or
(iv) a methylene group (CH₂);

L1 and L2 cannot simultaneously represent a covalent bond if X has only 1 carbon atom;

X1, X2, X3, X4, and X5, identical or different, independently represent a hydrogen or halogen atom, an NO₂, nitrile, alkyl, cycloalkyl, alkenyl, alkynyl, aryl, arylalkyl, —OR4, —SR4, —NR4R5, —SOR6, or —SO₂R6 group, a heterocycle, or a —Y-E type group, with at least one of the X1, X2, X3, X4, and X5 group being a —Y-E type group;

R4 and R5, identical or different, represent independently a hydrogen atom or an alkyl, cycloalkyl, alkenyl, alkynyl, aryl, arylalkyl group, a heterocycle, or R4 and R5 together may form, with the nitrogen atom they are linked to, a cycle or a heterocycle;

R6, substituted or not, independently represents an alkyl, cycloalkyl, alkenyl, alkynyl, aryl, arylalkyl group, or a heterocycle;

Y represents a methylene group substituted or not, an oxygen, sulfur, or selenium atom, a SO, SO₂, SeO, SeO₂, or NR group in which R represents a hydrogen atom, or an alkyl, cycloalkyl, alkenyl, alkynyl, aryl, arylalkyl group, or a heterocycle;

E represents an alkyl, cycloalkyl, alkenyl, or alkynyl chain, comprising or not one or several Y1 groups and substituted by one or several W groups,

Y1 represents an oxygen or sulfur atom, or a NR type group, R representing a hydrogen atom or an alkyl, cycloalkyl, alkenyl, alkynyl, aryl, or arylalkyl group, in particular a hydrogen atom or an alkyl radical;

W represents:
(i) a carboxylic acid (—COOH) or an ester (—COOR4), a thioester (—COSR4), an amide (—CONR4R5), a thioamide (—CSNR4R5), a nitrile (—CN) derivative, or
(ii) an acylsulfonamide group (—CONHSO₂R6), or
(iii) a tetrazole, or
(iv) an isoxazole, or
(v) a sulfonic acid (—SO₃H), or
(vi) a (—SO₃R4) or (—SO₂NR4R5) derivative, or
(vii) a hydrazide (—CONHNR4R5),

R4, R5, and R6 being as above-described;

their stereoisomers (diastereoisomers, enantiomers), pure or mixed, racemic mixtures, geometrical isomers, tautomers, salts, hydrates, solvates, solid forms and mixtures thereof.

In the context of the present invention, the term “alkyl” designates a hydrocarbon radical that is saturated, linear, branched, or cyclic, substituted or not, having from 1 to 24, and preferably from 1 to 10, carbon atoms (e.g. methyl, ethyl, n-propyl, isopropyl, n-butyl, isobutyl, tert-butyl, sec-butyl, pentyl, neopentyl, n-hexyl, or cyclohexyl).

The term “alkenyl” designates an unsaturated hydrocarbon radical (having at least one double bond), linear, branched or cyclic, substituted or not, having from 2 to 24, preferably 2 to 10, carbon atoms (e.g. ethenyl, 1-propenyl, 2-propenyl, 1-butenyl, 2-butenyl, 1-pentenyl, 2- pentenyl, 3-methyl-3-butenyl).

The term “alkynyl” designates an unsaturated hydrocarbon radical (having at least one triple bond), linear, branched or cyclic, substituted or not, having from 2 to 24, preferably 2 to 10, carbon atoms (e.g. ethenyl, 1-propenyl, 2-propenyl, 1-butenyl, 2-butenyl, 1-pentenyl, or 2- pentenyl).

The term “alkyloxy” refers to an alkyl chain linked to the molecule by means of an oxygen atom (ether bond). The term “alkyl” corresponds to the previously expressed definition (cite.g. methodoxy, ethoxy, n-propyloxy, isopropyloxy, n-butyloxy, isobutyloxy, tert-butyloxy, sec-butyloxy, or hexyloxy).

The term “alkylthio” refers to an alkyl chain linked to a molecule by means of a sulfur atom (thioether bond). The term “alkyl” corresponds to the previously given definition. For example, methylthio, ethylthio, n-propylthio, isopropylthio, n-butylthio, isobutylthio, tert-butylthio, sec-butylthio and hexylthio can be cited.

The term “aryl” designates an aromatic hydrocarbon radical, substituted or not, having preferably from 6 to 14 carbon atoms. It can possibly be substituted, in particular, by at least one halogen atom, an alkyl, hydroxyl, thiol, alkyloxy, or alkylthio radical, or a nitro function (NO₂). Preferably, aryl radicals according to the invention are chosen from among phenyl, naphthyl (e.g. 1-naphthyl or 2-naphthyl), biphenyl (e.g., 2-, 3-, or 4-biphenyl), anthryl, or fluorenyl. phenyl groups, substituted or not, are especially preferred.

The term “heteroaryl” designates an aromatic hydrocarbon radical having one or several heteroatoms such as nitrogen, sulfur, and oxygen, substituted or not. It can possibly be substituted particularly by at least one halogen atom, an alkyl (as defined above), hydroxyl, thiol, alkyloxy (as defined above), alkylthio (as defined above), or a nitro function (NO₂). For example, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, isoxazolyl, and oxazolyle groups, etc can be cited.

The term “arylalkyl” designates an alkyl type radical substituted by an aryl group. The terms “alkyl” and “aryl” correspond to the previously given definitions. Phenethyl groups, possibly substituted, are especially preferred.

The term “heterocycle” designates a monocyclic or polycyclic, saturated, unsaturated, or aromatic radical, substituted or not, having one or several heteroatoms such as nitrogen, sulfur, and oxygen. Advantageously, they can be substituted by at least one alkyl, alkenyl, aryl, alkyloxy, or alkylthio groups as previously defined or a halogen atom. Pyridyl, furyl, thienyl, isoxazolyl, oxadiazolyl, oxazolyl, benzimidazol, indolyl, benzofuranyl, thiazolotriazolyl, morpholinyl, piperidinyl, piperazinyl, 2-oxo-piperidin-1-yl, and 2-oxo-pyrrolidin-1-yl radicals are especially preferred.

The term “cycloalkyl” designates more particularly a hydrocarbon cycle, substituted or not, saturated or unsaturated, generally having from 3 to 24, preferably from 3 to 10, carbon atoms. Cycloalkyls specially include cyclohexyl, cyclopentyl, cyclopropyl, cyclobutyl, cycloheptyl, and norbornyl groups.

By the term “cycle”, it is more particularly understood a hydrocarbon cycle, substituted or not, possibly presenting at least one heteroatom (such as a nitrogen, sulfur, or oxygen atom, for example), saturated, unsaturated, or aromatic. Cycles specially include cycloalkyl, aryl, or heterocycle groups as defined above.

The term “halogen” designates chlorine, bromine, fluorine and iodine.

Sulfur atoms may, within the context of the present invention, be oxidized or not.

The so-defined radicals may be substituted, in particular, by at least one halogen atom, an alkyl, cycloalkyl, aryl, hydroxyl, thiol, alkyloxy, alkylthio, hydroxyl, or heterocycle radical, or a nitro (NO₂) function. Hence, the alkyl group can be a perhalogenoalkyl radical, in particular perfluoroalkyl, such as —CF₃.

X represents an alkyl group whose principal chain has 1, 2, 3, 4, 5, or 6 carbon atoms or X represents an alkenyl or alkynyl group whose principal chain has 2, 3, 4, 5, or 6 carbon atoms.

A particular aspect of the invention relates to compounds of general formula (I) in which L1 represents a group of formula (II) defined as follows:

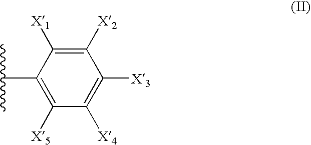

in which X′1, X′2, X′3, X′4, and X′5 are such as previously defined.

Preferably, compounds of formula (I) present a L1 group of formula (II) defined as follows:

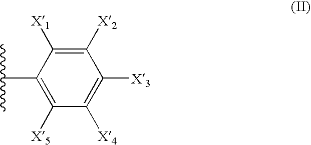

in which X′1, X′2, X′4, and X′5 are such as previously defined, and X′3 represents the L2 group.

Preferably, compounds of formula (I) present a L1 group of formula (II) defined as follows:

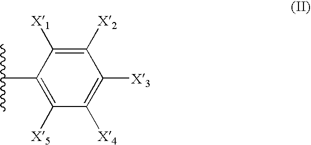

in which X′1, X′2, X′4, and X′5 represent a hydrogen atom, a nitro function (—NO₂), a trifluoromethyl radical (—CF3), an alkoxy group, preferably methoxy, or an alkyl radical, preferably methyl, ethyl or propyl, and X′3 represents the L2 group.

Preferably, compounds of formula (I) present a L1 group of formula (II) defined as follows:

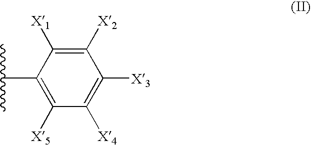

in which X′1, X′2, X′4, and X′5 represent a hydrogen atom and X′3 represents the L2 group.

Another aspect of the invention relates to compounds of general formula (I) in which L2 represents a covalent bond.

A preferred aspect of the invention relates to compounds of general formula (I) in which L2 represents a covalent bond and L1 represents a group of formula (II) as defined above.

Even more preferably, L1 represents a group of formula (II) as defined above and L2 represents a covalent bond in para position, with respect to X. Hence, the invention relates to compounds of general formula (III):

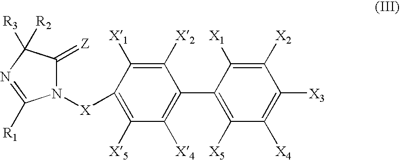

in which R1, R2, R3, Z, X, X1, X2, X3, X4, X5, X′1, X′2, X′4, and X′5 are as previously defined.

Another aspect of the invention relates to compounds of general formula (I) in which L2 represents a carbonyl group (CO).

According to a preferred aspect, the invention relates to compounds of general formula (I) in which L1 represents a group of formula (II) as defined above and L2 represents a carbonyl group (CO).

Even more preferably, L1 represents a group of formula (II) as defined above and L2 represents a carbonyl group (CO) in para position, with respect to X. Hence, the invention relates to compounds of general formula (IV):

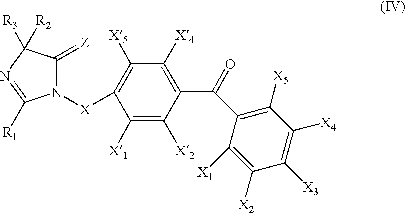

in which R1, R2, R3, Z, X, X1, X2, X3, X4, X5, X′1, X′2, X′4, and X′5 are as previously defined.

Another preferred aspect of the invention relates to compounds of general formula (I) in which L2 represents an oxygen atom. More preferably, the invention relates to compounds of general formula (I) in which L1 represents a group of formula (II) as defined above and L2 represents an oxygen atom.

Even more preferably, L1 represents a group of formula (II) as defined above and L2 represents an oxygen atom in para position, with respect to X. Hence, the invention relates to compounds of general formula (V):

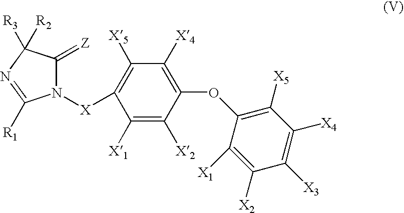

in which R1, R2, R3, Z, X, X1, X2, X3, X4, X5, X′1, X′2, X′4, and X′5 are as previously defined.

Another preferred aspect of the invention relates to compounds of general formula (I) in which L2 represents a sulfur atom. More preferably, the invention relates to compounds of general formula (I) in which L1 represents a group of formula (II) as defined above and L2 represents a sulfur atom (oxidized or not).

Even more preferably, L1 represents group of formula (II) as defined above and L2 represents a sulfur atom (oxidized or not) in para position, with respect to X. Hence, the invention relates to compounds of general formula (VI):

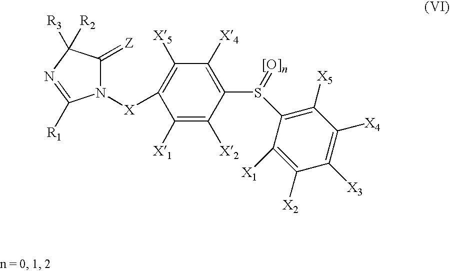

in which R1, R2, R3, Z, X, X1, X2, X3, X4, X5, X′1, X′2, X′4, and X′5 are as previously defined.

Another preferred aspect of the invention relates to compounds of general formula (I) in which L2 represents a methylene group. More preferably, the invention relates to compounds of general formula (I) in which L1 represents a group of formula (II) as defined above and L2 represents a methylene group.

Even more preferably, L1 represents a group of formula (II) as defined above and L2 represents a methylene group situated in para position, with respect to X. Hence, the invention relates to compounds of general formula (VII):

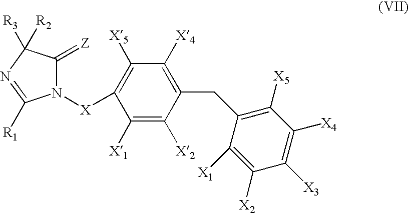

in which R1, R2, R3, Z, X, X1, X2, X3, X4, X5, X′1, X′2, X′4, and X′5 are as previously defined.

Another distinctive aspect of the invention relates to compounds of general formula (I) in which L1 represents a covalent bond and L2 is such as above defined.

Preferably, the invention relates to compounds of general formula (I) in which L1 and L2 simultaneously represent a covalent bond and in which X has more than one carbon atom.

Hence, the invention relates to compounds of general formula (VIII):

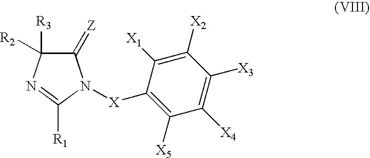

in which R1, R2, R3, Z, X, X1, X2, X3, X4, and X5 are such as previously defined and in which X is such as previously defined and has more than one carbon atom.

Another distinctive aspect of the invention relates to compounds of formula (I) in which L1 represents a group of formula (II) defined as follows:

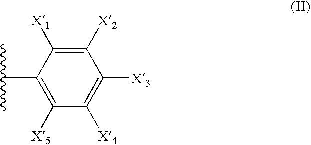

in which X′1, X′3, X′4, and X′5 are such as previously defined, and X′2 represents the L2 group.

Preferably, compounds of formula (I) present a L1 group of formula (II) defined as follows:

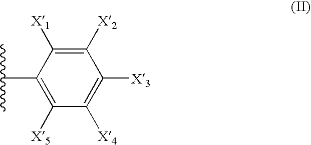

in which X′1, X′3, X′4, and X′5 represent a hydrogen atom and X′2 represents the L2 group.

Another aspect of the invention relates to compounds of general formula (I) in which L2 represents a covalent bond.

A preferred aspect of the invention relates to compounds of general formula (I) in which L2 represents a covalent bond and L1 represents a group of formula (II) as above defined.

Even more preferably, L1 represents a group of formula (II) as defined above and L2 represents a covalent bond in meta position, with respect to X. Hence, the invention relates to compounds of general formula (IX):

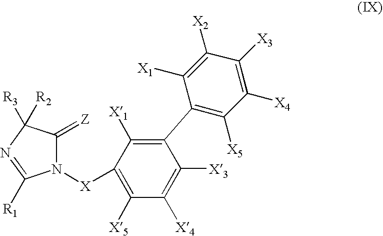

in which R1, R2, R3, Z, X, X1, X2, X3, X4, X5, X′1, X′3, X′4, and X′5 are such as previously defined.

Another distinctive aspect of the invention relates to the general formula (I) compounds in which L1 represents a formula (IX) group defined as follows.

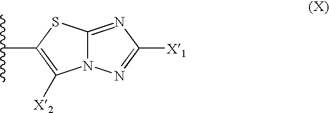

in which X′1 and X′2 are such as previously defined.

Preferably, compounds of formula (I) present a L1 group of formula (X) group defined as follows:

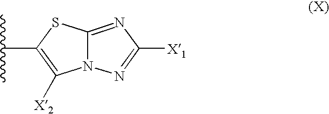

in which X′2 is such as previously defined, and X′1 represents the L2 group.

Preferably, compounds of formula (I) present a L1 group of formula (X) defined as follows:

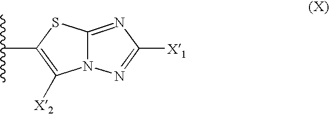

in which X′2 is a methyl and X′1, represents the L2 group.

Another aspect of the invention relates to compounds of general formula (I) in which L2 represents a covalent bond.

A preferred aspect of the invention relates to compounds of general formula (I) in which L2 represents a covalent bond and L1 represents a group of formula (X) as defined above.

Even more preferably, L1 represents a group of formula (X) as defined above and X′1 represents the L2 group, the L2 group being a covalent bond. Hence, the invention relates to general formula (XI) compounds:

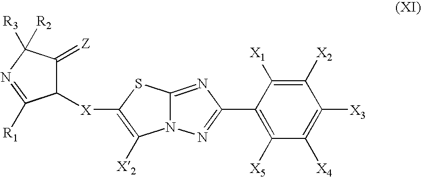

in which R1, R2, R3, Z, X, X1, X2, X3, X4, X5, and X′2 are such as previously defined.

A particular subject-matter of the invention relates to compounds of general formula (I), preferably (III), (IV), (V), (VI), (VII), (VIII), (X), or (XI) in which R1 represents an alkyl, cycloalkyl, alkenyl, alkynyl, aryl, arylalkyl, or a heterocycle group, preferably an alkyl group.

More preferably, R1 represents an alkyl group, substituted or not, having in its principal chain preferably 1, 2, 3, 4, 5, or 6 carbon atoms. R1 can be substituted by an aryl or cycloalkyl group possibly having a heteroatom.

R1 can, for example, represent a butyl, isobutyl, ethyl, methyl, cyclopropyl, or methyl substituted by a phenyl group or by a thiophenyl group. Even more preferably, R1 represents a butyl group.

A particular subject-matter of the invention relates to compounds of general formula (I), advantageously (III), (IV), (V), (VI), (VIl), (VIII), (X), or (XI) in which R2 and R3, identical or different, independently represent an alkyl group having preferably 1, 2, 3, 4, 5, or 6 carbon atoms or an arylalkyl group, or in which R2 and R3 form a cycle with the carbon they are bonded to, preferably a cycle having from 3 to 8 carbon atoms. The cycle formed by R2, R3, and the carbon which they are bonded to can have 3, 4, 5, 6, 7, or 8 carbon atoms.

More preferably, R2 and R3, identical or different, independently represent a hydrogen atom, a methyl, ethyl or phenyl group, or R2 and R3 form, with the carbon they are bonded to, a cycle having 5 or 6 carbon atoms, preferably a cyclopentyl or a cyclohexyl.

A particular subject-matter of the invention relates to general formula (I) compounds, advantageously (III), (IV), (V), (VI), (VII), (VIII), (IX), or (XI) in which Z represents an oxygen atom.

A particular subject-matter of the invention relates to general formula (I) compounds, advantageously (III), (IV), (V), (VI), (VIl), (VIII), (IX), or (XI) in which X represents an alkyl group in which the principal chain has 1 or 2 carbon atoms, preferably non-substituted.

A particular subject-matter of the invention relates to general formula (I) compounds, advantageously (III), (IV), (V), (VI), (VII), (VIII), (IX), or (XI) in which X1, X2, X3, X4, and X5, identical or different, independently represent a hydrogen atom, a halogen atom, preferably bromine or fluorine, an alkyle group—preferably propyl, ethyl, isobutyl-, an alkyloxy -preferably methoxy-, a nitrile (CN), a nitro (NO₂), or a —Y-E group as previously defined, at least one of the groups X1, X2, X3, X4, and X5 being a —Y-E group.

Within the context of the present invention, the position of the Y-E group(s) can be in ortho (X1 and/or X5=Y-E), meta (X2 and/or X4=Y-E) and/or para (X3=Y-E) of the aromatic cycle to which it(they) is(are) bonded, with respect to the L2 group.

Preferably, only one of the groups X1, X2, X3, X4, and X5 represents a —Y-E group. Even more preferably, X2 or X4 represents the Y-E (the Y-E group is then in meta position of the aromatic cycle to which it is bonded), X1, X3, X5, and X4 or X2, respectively, possibly representing a hydrogen atom, a halogen atom, an alkyl, alkyloxy, nitrile or a nitro group (NO₂).

A particular subject-matter of the invention relates to compounds of general formula (I), advantageously (III), (IV), (V), (VI), (VIl), (VIII), (IX), or (XI) in which at least 3 of the groups X1, X2, X3, X4, and X5 represent a hydrogen atom.

A particular subject-matter of the invention relates to compounds of general formula (I), advantageously (Ill), (IV), (V), (VI), (VII), (VIII), (IX), or (XI) in which at least one of the groups X1, X2, X3,. X4, and X5 represents a halogen atom, preferably bromine or fluorine.

A particular subject-matter of the invention relates to compounds of general formula (I), advantageously (III), (IV), (V), (VI), (VII), (VIII), (IX), or (XI) in which at least one of the groups X1, X2, X3, X4, and X5 represents an alkyl chain, preferably ethyl, propyl or isobutyl.

A particular subject-matter of the invention relates to compounds of general formula (I), advantageously (III), (IV), (V), (VI), (VII), (VIII), (IX), or (XI) in which at least one of the groups X1, X2, X3, X4, and X5 represents an alkoxy group, preferably methoxy.

A particular subject-matter of the invention relates to compounds of general formula (I), advantageously (III), (IV), (V), (VI), (VII), (VIII), (IX), or (XI) in which at least one of the groups X1, X2, X3, X4, and X5 represents a nitrile group.

A particular subject-matter of the invention relates to compounds of general formula (I), advantageously (III), (IV), (V), (VI), (VIl), (VIII), (IX), or (XI) in which at least one of the groups X1, X2, X3, X4, and X5 represents a nitro group (NO₂).

A particular subject-matter of the invention relates to compounds of general formula (I), advantageously (III), (IV), (V), (VI), (VII), (VIII), (IX), or (XI) in which Y represents an oxygen atom.

A particular subject-matter of the invention relates to general formula (I) compounds, advantageously (III), (IV), (V), (VI), (VIl), (VIII), (IX), or (XI) in which E represents a principal alkyl chain, branched or not, having preferably 1, 2, 3, 4, 5, 6, 7, 8, or 9 carbon atoms, substituted by one or several W groups as above-defined, preferably by only one W group.

A particular subject-matter of the invention relates to general formula (I) compounds, advantageously (III), (IV), (V), (VI), (VII), (VIII), (IX), or (XI) in which the W group represents a carboxylic acid (—COOH) or an ester (COOR4), a thioester (—COSR4), an amide (—CONR4R5), a thioamide (—CSNR4R5), a nitrile (—CN), an acylsulfonamide (—CONHSO₂R6), a hydrazide (—CONHNR4R5), or a tetrazole; R4, R5, and R6 being as previously described.

Preferably, W represents a carboxylic acid (—COOH) or an ester (—COOR4), a nitrile (—CN), or a tetrazole.

A particular subject-matter of the invention relates to compounds of general formula (I), advantageously (III), (IV), (V), (VI), (VII), (VIII), (IX), or (XI) in which the —Y-E group represents —O—C(CH₃)₂—COOH, —O—(CH₂)₃—C(CH₃)₂—COOH, —O—CH₂—CN, —O—CH₂—C(CH₃)₂—COOH, —O—(CH₂)₆—C(CH₃)₂—COOH, —O—CH₂—COOH, —O—CH(CH₃)—COOH, —O—CH(CH₂CH₃)—COOH, —O—CH(CH(CH₃)₂)—COOH, O—CH₂-tetrazole, —O—CH(CH₂CH₃)tetrazole, —O—C(spirocyclobutyle)—COOH.

Even more preferably, the invention is directed to compounds of general formula (I) in which at least one, and preferably all, of the following conditions are met:

R1 represents an alkyl group, substituted or not, having in its principal chain preferably 1, 2, 3, 4, 5, or 6 carbon atoms; and/or

R2 and R3, identical or different, independently represent an alkyl group, preferably having 1, 2, 3, 4, 5, or 6 carbon atoms or an arylalkyl group, or R2 and R3, with the carbon to which they are bonded, form a cycle having from 3 to 8 carbon atoms; and/or

Z represents an oxygen atom; and/or

X represents an alkyl group, in which the principal chain comprises 1 or 2 carbon atoms; and/or

L1 represents:
(i) a covalent bond, or
(ii) a group of formula (II) defined as follows:

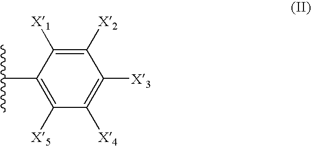

X′1, X′2, X′4, and X′5, identical or different, independently represent a hydrogen atom, a halogen atom, or an alkyl chain;

X′3 representing L2;

or alternatively X′1, X′2, X′4, and X′5, identical or different, independently represent a hydrogen atom, a halogen atom, or an alkyl chain;

X′2 representing L2; and/or

L2 represents:
(i) a covalent bond, or
(ii) a carbonyl group (CO), or
(iii) an oxygen or sulfur atom; or
(iv) a methylene group (CH₂););

L1 and L2 do not simultaneously represent a covalent bond if X has only 1 carbon atom; and/or

X1, X2, X3, X4, and X5, identical or different, independently represent a hydrogen atom, a halogen atom, an alkyl chain, an alkoxy, nitrile, nitro (—NO₂) group, or a —Y-E group, with at least, preferably only one, of the groups X1, X2, X3, X4, and X5 being a —Y-E group; and/or

Y represents an oxygen atom; and/or

E represents an alkyl principal chain, branched or not, having preferably 1, 2, 3, 4, 5, 6, 7, 8, or 9 carbon atoms, substituted by one or several W groups; and/or

W represents a —COOH carboxylic acid or an ester (—COOR4), nitrile (—CN), or tetrazole;

their stereoisomers (diastereoisomers, enantiomers), pure or mixed, racemic mixtures, geometrical isomers, tautomers, salts, hydrates, solvates, solid forms and mixtures thereof.

In accordance with a particular embodiment of the invention, the preferred compounds are indicated below:

### Compound 1: 2-butyl-1-[(3′-((1-carboxy-1,1-dimethylmethyl)oxy)biphenyl-4-yl)methyl]-4-spirocyclopentyl-1H-imidazol-5(4H)-one

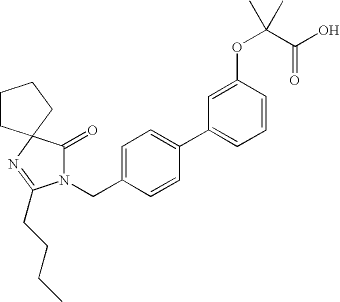

### Compound 2: 2-butyl-1-[(4′-((1-carboxy-1,1-dimethylmethyl)oxy)biphenyl-4-yl)methyl]-4-spirocyclopentyl-1H-imidazol-5(4H)-one

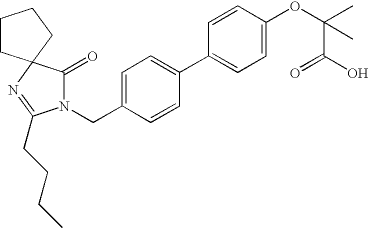

### Compound 3: 2-butyl-1-[2-(4-((1-carboxy-1,1-dimethylmethyl)oxy)phenyl)ethyl]-4-spirocyclopentyl-1H-imidazol-5(4H)-one

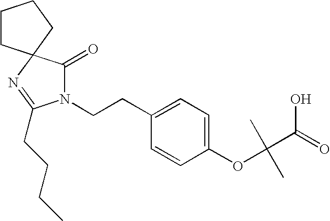

### Compound 4: 1-[(5′-bromo-2′-((1-carboxy-1,1-dimethylmethyl)oxy)biphenyl-4-yl)methyl]-2-butyl-4-spirocyclopentyl-1H-imidazol-5(4H)-one

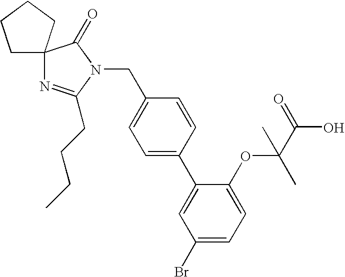

### Compound 5: 2-butyl-1-[[4-[(3-((1-carboxy-1,1-dimethylmethyl)oxy)phenyl)carbonyl]phenyl]methyl]-4-spirocyclopentyl-1H-imidazol-5(4H)-one

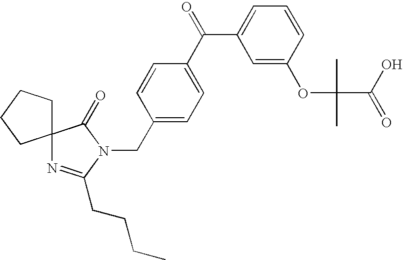

### Compound 6: 2-butyl-1-[(2′-((1-carboxy-1,1-dimethylmethyl)oxy)biphenyl-4-yl)methyl]-4-spirocyclopentyl-1H-imidazol-5(4H)-one

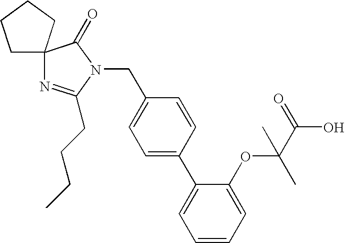

### Compound 7: 2-butyl-1-[[4-[(2-((1-carboxy-1,1-dimethylmethyl)oxy)phenyl)carbonyl]phenyl]methyl]-4-spirocyclopentyl-1H-imidazol-5(4H)-one

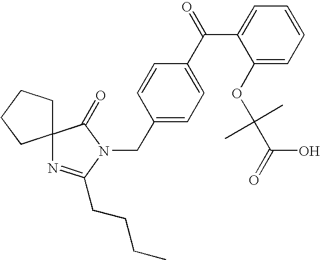

### Compound 8: 2-butyl-1-[2-(3-((1-carboxy-1,1-dimethylmethyl)oxy)phenyl)ethyl]-spirocyclopentyl-1H-imidazol-5(4H)-one

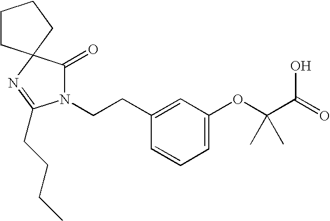

### Compound 9: 2-butyl-1-[[4-[(4-((1-carboxy-1,1-dimethylmethyl)oxy)phenyl)carbonyl]phenyl]methyl]-4-spirocyclopentyl-1H-imidazol-5(4H)-one

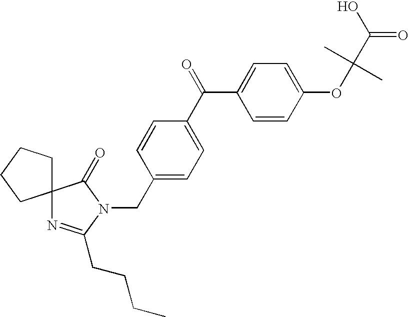

### Compound 10: 2-butyl-1-[2-(2-((1-carboxy-1,1-dimethylmethyl)oxy)phenyl)ethyl]-4-spirocyclopentyl-1H-imidazol-5(4H)-one

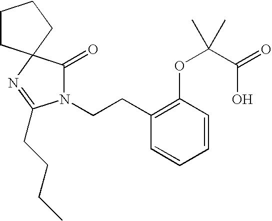

### Compound 11: 2-butyl-1-[[4-[(3-((1-carboxy-1,1-dimethylmethyl)oxy)phenyl)carbonyl]phenyl]methyl]-4-spirocyclohexyl-1H-imidazol-5(4H)-one

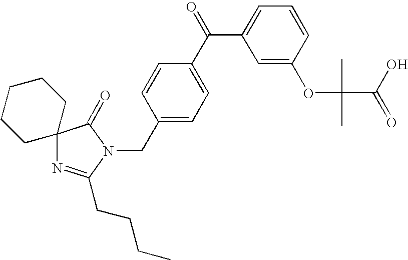

### Compound 12: 1-[(6′-bromo-3′-((1-carboxy-1,1-dimethylmethyl)oxy)biphenyl-4-yl)methyl]-2-butyl-4-spirocyclopentyl-1H-imidazol-5(4H)-one

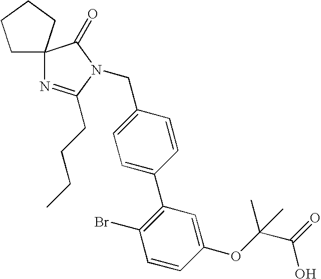

### Compound 13: 2-butyl-1-[(3′-((1-carboxy-1,1-dimethylmethyl)oxy)biphenyl-4-yl)methyl]-4,4-dimethyl-1H-imidazol-5(4H)-one

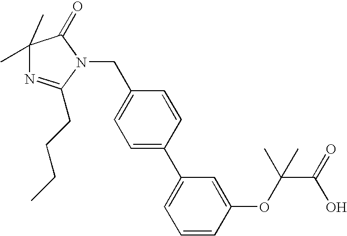

### Compound 14: 2-butyl-1-[[4-[(3-((1-carboxy-1,1-dimethylmethyl)oxy)phenyl)carbonyl]phenyl]methyl]-4,4-dimethyl-1H-imidazol-5(4H)-one

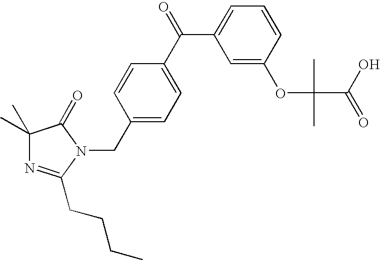

### Compound 15: 2-butyl-1-[[4-[(3-((1-carboxy-1,1-dimethylmethyl)oxy)phenyl)carbonyl]phenyl]methyl]-4-phenyl-1H-imidazol-5(4H)-one

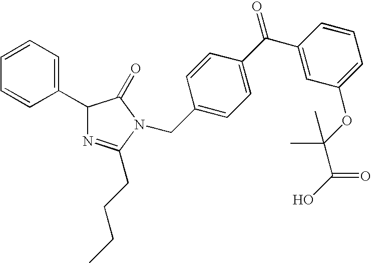

### Compound 16: 1-[(3′-((1-carboxy-1,1-dimethylmethyl)oxy)biphenyl-4-yl)methyl]-2-propyl-4-spirocyclopentyl-1H-imidazol-5(4H)-one

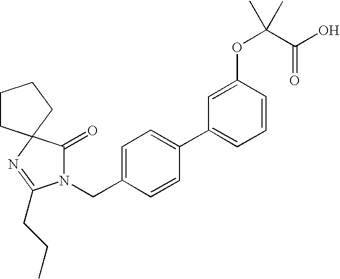

### Compound 17: 1-[(3′-((1-carboxy-1,1-dimethylmethyl)oxy)biphenyl-4-yl)methyl]-2-ethyl-4-spirocyclopentyl-1H-imidazol-5(4H)-one

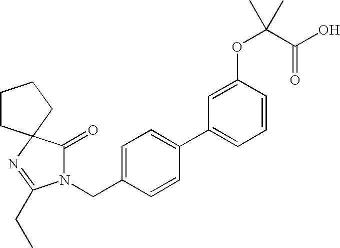

### Compound 18: 1-[(3′-((1-carboxy-1,1-dimethylmethyl)oxy)biphenyl-4-yl)methyl]-2-methyl-4-spirocyclopentyl-1H-imidazol-5(4H)-one

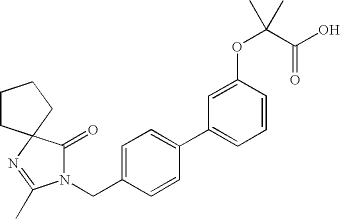

### Compound 19: 2-butyl-1-[(3′-((1-carboxy-1,1-dimethylmethyl)oxy)biphenyl-4-yl)methyl]-4-phenyl-1H-imidazol-5(4H)-one

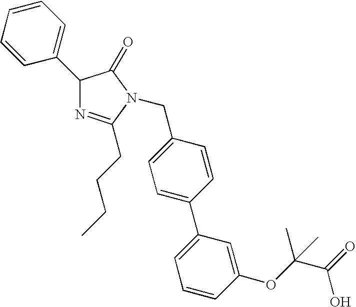

### Compound 20: 2-butyl-1-[[4-[(4-((1-carboxy-1,1-dimethylmethyl)oxy)phenyl)carbonyl]phenyl]methyl]-4-phenyl-1H-imidazol-5(4H)-one

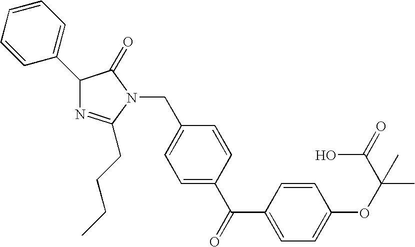

### Compound 21: 2-butyl-1-[(3′-((1-carboxy-1,1-dimethylmethyl)oxy)biphenyl-4-yl)methyl]-4-spirocyclopentyl-1H-imidazol-5(4H)-one

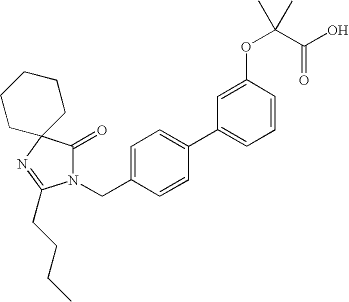

### Compound 22: 1-[(6′-bromo-3′-((1-carboxy-1,1-dimethylmethyl)oxy)biphenyl-4-yl)methyl]-2-butyl-4-spirocyclohexyl-1H-imidazol-5(4H)-one

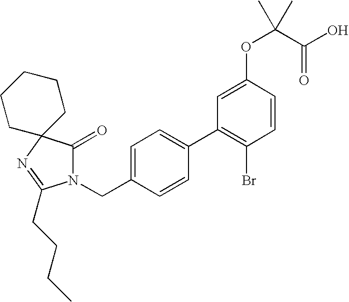

### Compound 23: 2-butyl-1-[(3′-(cyanomethoxy)biphenyl-4-yl)methyl]-4-spirocyclopentyl-1H-imidazol-5(4H)-one

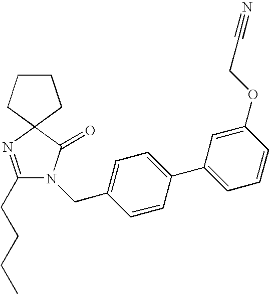

### Compound 24: 1-[(5′-bromo-2′-((1-carboxy-1,1-dimethylmethyl)oxy)biphenyl-4-yl)methyl]-2-butyl-4-spirocyclohexyl-1H-imidazol-5(4H)-one

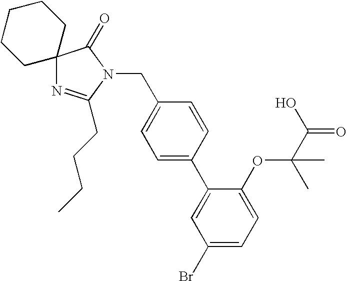

### Compound 25: 2-butyl-1-[(4′-((1-carboxy-1,1-dimethylmethyl)oxy)biphenyl-4-yl)methyl]-4-spirocyclopentyl-1H-imidazol-5(4H)-one

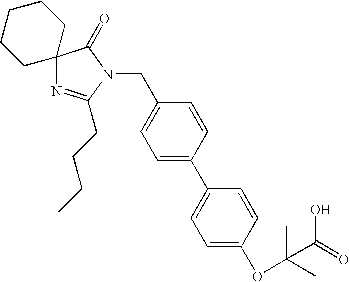

### Compound 26: 1-[(5′-bromo-2′-((1-carboxy-1,1-dimethylmethyl)oxy)biphenyl-4-yl)methyl]-2-butyl-4-phenyl-1H-imidazol-5(4H)-one

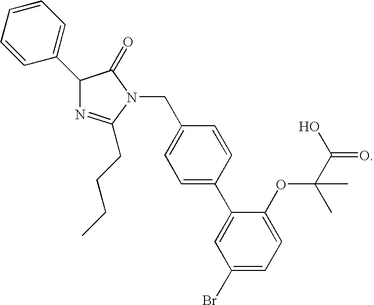

### Compound 27: 1-[(3′-((1-carboxy-1,1-dimethylmethyl)oxy)biphenyl-4-yl)methyl]-2-(2-methyl)propyl-4-spirocyclopentyl-1H-imidazol-5(4H)-one

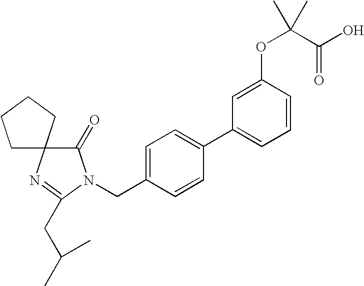

### Compound 28: 2-benzyl-1-[(3′-((1-carboxy-1,1-dimethylmethyl)oxy)biphenyl-4-yl)methyl]-4-spirocyclopentyl-1H-imidazol-5(4H)-one

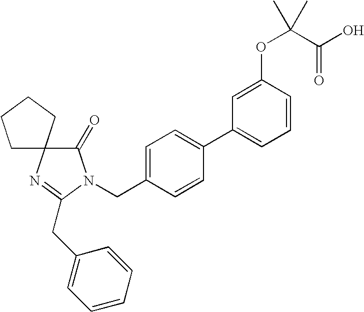

### Compound 29: 1-[(3′-((1-carboxy-1,1-dimethylmethyl)oxy)biphenyl-4-yl)methyl]-2-cyclopropyl-4-spirocyclopentyl-1H-imidazol-5(4H)-one

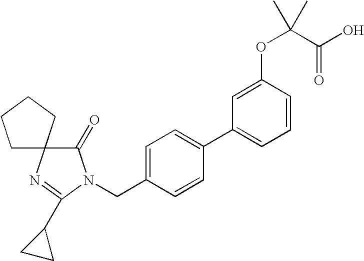

### Compound 30: 1-[(3′-((1-carboxy-1,1-dimethylmethyl)oxy)biphenyl-4-yl)methyl]-2-(thiophen-2-yl)methyl-4-spirocyclopentyl-1H-imidazol-5(4H)-one

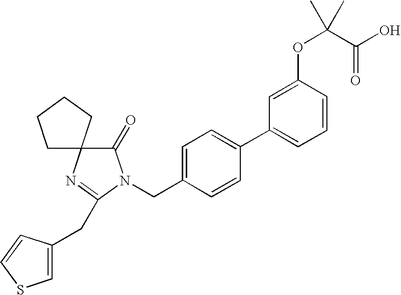

### Compound 31: 2-butyl-1-[[4-[(4-(1-carboxy-1,1-dimethylmethyloxy)phenyl)methyl]phenyl]methyl]-4-spirocyclopentyl-1H-imidazol-5(4H)-one

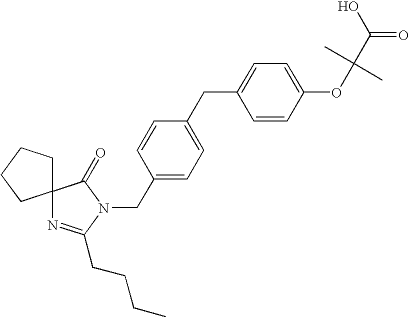

### Compound 32: 2-butyl-1-[(4′-((4-carboxy-4,4-dimethylbutan-1-yl)oxy)biphenyl-4-yl)methyl]-4-spirocyclopentyl-1H-imidazol-5(4H)-one

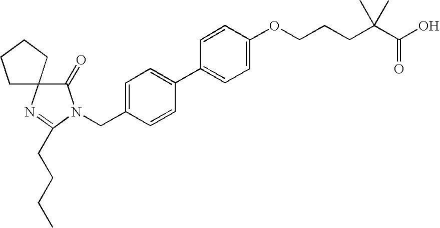

### Compound 33: 2-butyl-1-[(3′-((4-carboxy-4,4-dimethylbutan-1-yl)oxy)biphenyl-4-yl)methyl]-4-spirocyclopentyl-1H-imidazol-5(4H)-one

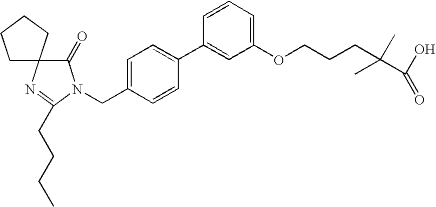

### Compound 34: 2-butyl-1-[[4-[(4-(1-carboxy-1,1-dimethylmethyloxy)phenyl)oxy]phenyl]methyl]-4-spirocyclopentyl-1H-imidazol-5(4H)-one

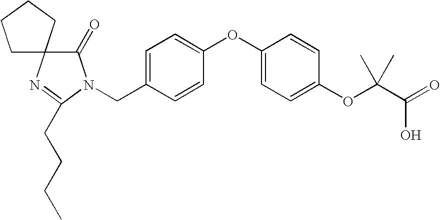

### Compound 35: 2-butyl-1-[[4-[(4-(1-carboxy-1,1-dimethylmethyloxy)phenyl)oxy]phenyl]methyl]-4-spirocyclohexyl-1H-imidazol-5(4H)-one

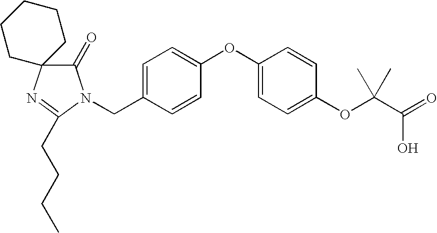

### Compound 36: 2-butyl-1-[[4-[(3-(1-carboxy-1,1-dimethylmethyloxy)phenyl)methyl]phenyl]methyl]-4-spirocyclopentyl-1H-imidazol-5(4H)-one

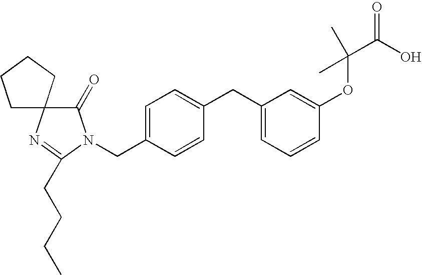

### Compound 37: 2-butyl-1-[[4-[(3-(1-carboxy-1,1-dimethylmethyloxy)phenyl)methyl]phenyl]methyl]-4-spirocyclohexyl-1H-imidazol-5(4H)-one

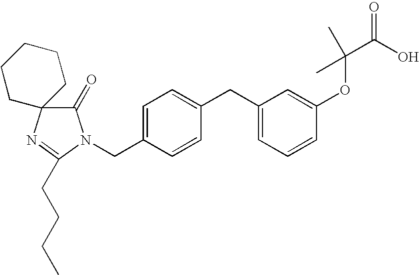

### Compound 38: 2-butyl-1-[(4′-((4-carboxy-4,4-dimethylbutan-1-yl)oxy)biphenyl-4-yl)methyl]-4-spirocyclohexyl-1H-imidazol-5(4H)-one

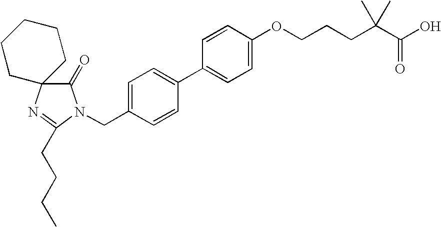

### Compound 39: 2-butyl-1-[[4-[(3-(1-carboxy-1,1-dimethylmethyloxy)phenyl)oxy]phenyl]methyl]-4-spirocyclohexyl-1H-imidazol-5(4H)-one

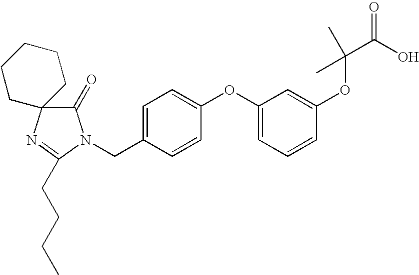

### Compound 40: 2-butyl-1-[[4-[(3-(1-carboxy-1,1-dimethylmethyloxy)phenyl)oxy]phenyl]methyl]-4-spirocyclopentyl-1H-imidazol-5(4H)-one

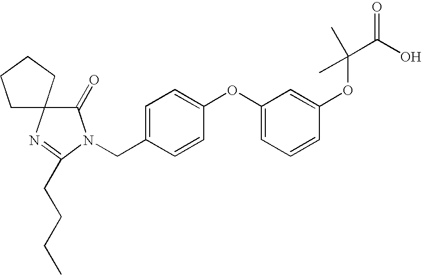

### Compound 41: 2-butyl-1-[[4-[(2-(1-carboxy-1,1-dimethylmethyloxy)phenyl)methyl]phenyl]methyl]-4-spirocyclopentyl-1H-imidazol-5(4H)-one

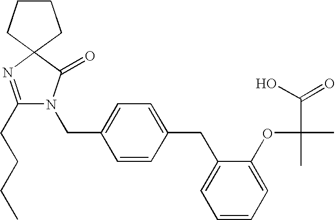

### Compound 42: 2-butyl-1-[(2′-((4-carboxy-4,4-dimethylbutan-1-yl)oxy)biphenyl-4-yl)methyl]-4-spirocyclohexyl-1H-imidazol-5(4H)-one

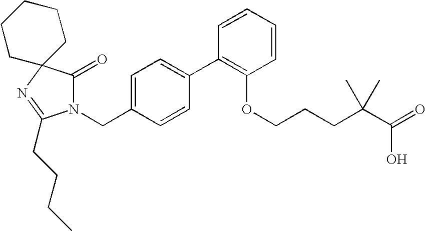

### Compound 43: 2-butyl-1-[(2′-((7-carboxy-7,7-dimethylheptan-1-yl)oxy)biphenyl-4-yl)methyl]-4-spirocyclopentyl-1H-imidazol-5(4H)-one

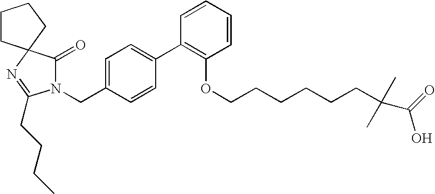

### Compound 44: 2-butyl-1-[(2′-((4-carboxy-4,4-dimethylbutan-1-yl)oxy)biphenyl-4-yl)methyl]-4-spirocyclopentyl-1H-imidazol-5(4H)-one

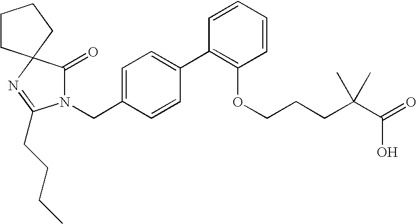

### Compound 45: 2-butyl-1-[[4-[(2-(1-carboxy-1,1-dimethylmethyloxy)phenyl)methyl]phenyl]methyl]-4-spirocyclohexyl-1H-imidazol-5(4H)-one

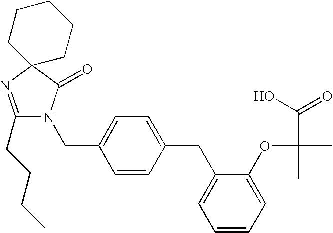

### Compound 46: 2-butyl-1-[[4-[(3-(1-carboxy-1,1-dimethylmethyloxy)phenyl)thio]phenyl]methyl]-4-spirocyclopentyl-1H-imidazol-5(4H)-one

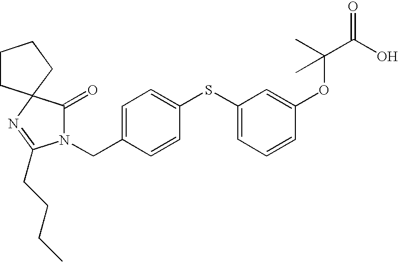

### Compound 47: 2-butyl-1-[[4-[(3-(1-carboxy-1,1-dimethylmethyloxy)phenyl)thio]phenyl]methyl]-4-spirocyclopentyl-1H-imidazol-5(4H)-one

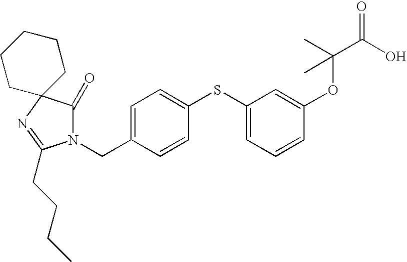

### Compound 48: 2-butyl-1-[[4-[(4-(1-carboxy-1,1-dimethylmethyloxy)phenyl)thio]phenyl]methyl]-4-spirocyclohexyl-1H-imidazol-5(4H)-one

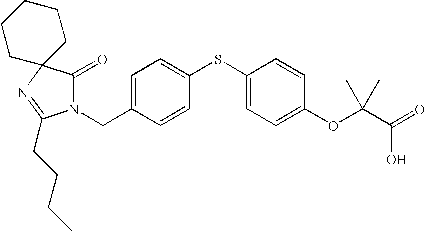

### Compound 49: 2-butyl-1-[(3′-((2-carboxy-2,2-dimethylethyl-1-yl)oxy)biphenyl-4-yl)methyl]-4-spirocyclopentyl-1H-imidazol-5(4H)-one

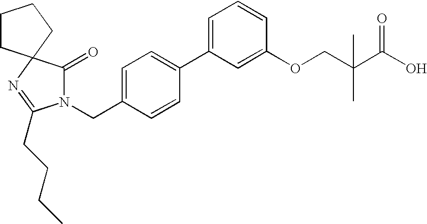

### Compound 50: 2-butyl-1-[[4-[(4-(1-carboxy-1,1-dimethylmethyloxy)phenyl)methyl]phenyl]methyl]-4-spirocyclohexyl-1H-imidazol-5(4H)-one

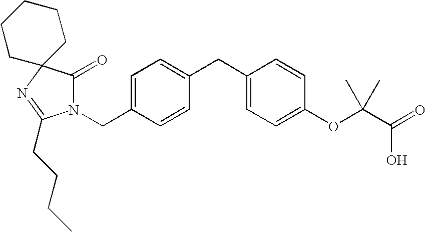

### Compound 51: 2-butyl-1-[[4-[(4-(1-carboxy-1,1-dimethylmethyloxy)phenyl)thio]phenyl]methyl]-4-spirocyclopentyl-1H-imidazol-5(4H)-one

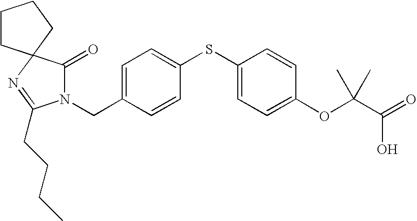

### Compound 52: 2-butyl-1-[(3′-((1-carboxymethyl)oxy)biphenyl-4-yl)methyl]-4-spirocyclohexyl-1H-imidazol-5(4H)-one

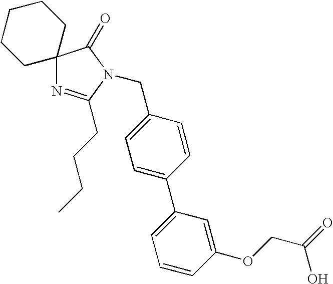

### Compound 53: 2-butyl-1-[(3′-((1-carboxy-1-methylmethyl)oxy)biphenyl-4-yl)methyl]-4-spirocyclohexyl-1H-imidazol-5(4H)-one

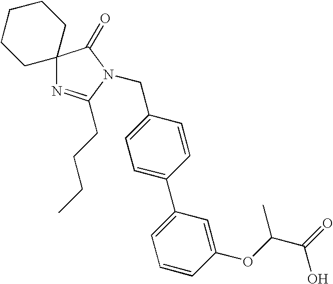

### Compound 54: 2-butyl-1-[(3′-((1-carboxy-1-ethylmethyl)oxy)biphenyl-4-yl)methyl]-4-spirocyclohexyl-1H-imidazol-5(4H)-one

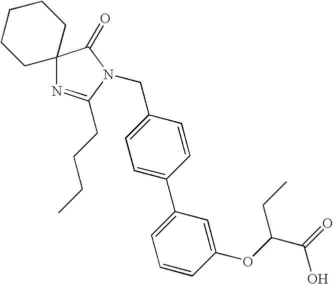

### Compound 55: 2-butyl-1-[(3′-((1-carboxy-1-(1,1-dimethylmethyl)methyl)oxy)-biphenyl-4-yl)methyl]-4-spirocyclohexyl-1H-imidazol-5(4H)-one

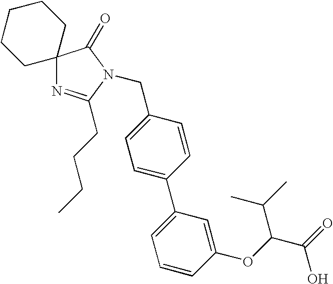

### Compound 56: 2-butyl-1-[(3′-((1-carboxymethyl)oxy)-6′-fluoro-biphenyl-4-yl)methyl]-4-spirocyclo hexyl-1H-imidazol-5(4H)-one

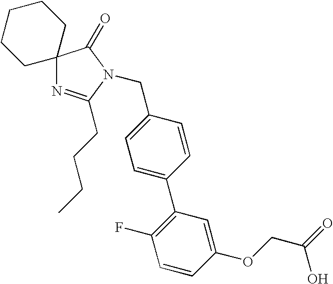

### Compound 57: 2-butyl-1-[(3′-((1-carboxy-1-methylmethyl)oxy)-6′-fluoro-biphenyl-4-yl)methyl]-4-spirocyclohexyl-1H-imidazol-5(4H)-one

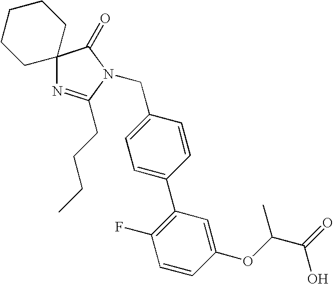

### Compound 58: 2-butyl-1-[(3′-((1-carboxy-1-ethylmethyl)oxy)-6′-fluoro-biphenyl-4-yl)methyl]-4-spirocyclohexyl-1H-imidazol-5(4H)-one

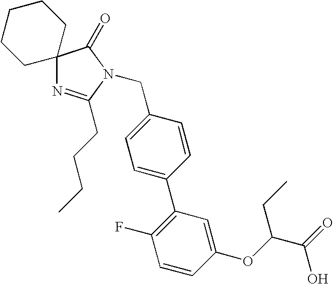

### Compound 59: 2-butyl-1-[(3′-((1-carboxy-1-(1,1-dimethylmethyl)methyl)oxy)-6′-fluoro-biphenyl-4-yl)methyl]-4-spirocyclohexyl-1H-imidazol-5(4H)-one

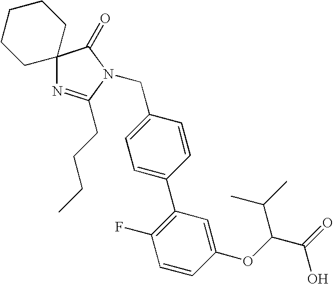

### Compound 60: 2-butyl-1-[(3′-((1-carboxy-1,1-dimethylmethyl)oxy)-6′-fluoro-biphenyl-4-yl)methyl]-4-spirocyclohexyl-1H-imidazol-5(4H)-one

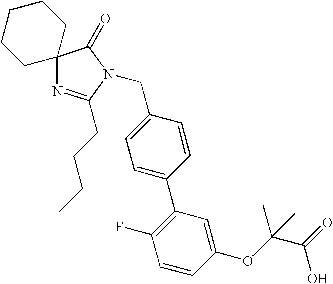

### Compound 61: 2-butyl-4-spirocyclohexyl-1-[(3′-((1-(tetrazol-5-yl)methyl)oxy)biphenyl-4-yl)methyl]-1H-imidazol-5(4H)-one

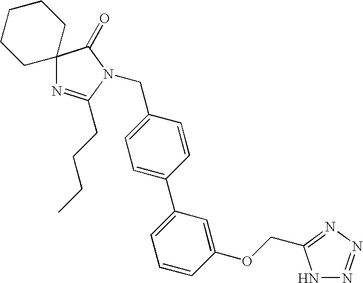

### Compound 62: 2-butyl-1-[(6′-fluoro-3′-((1-(tetrazol-5-yl)methyl)oxy)biphenyl-4-yl)methyl]-4-spirocyclohexyl-1H-imidazol-5(4H)-one

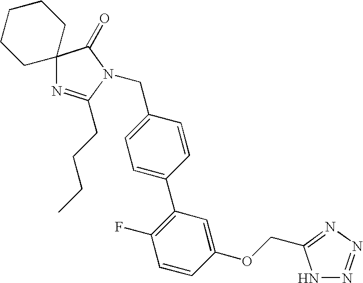

### Compound 63: 2-butyl-1-[(3′-((1-carboxy-1-(1,1-dimethylmethyl)methyl)oxy)-biphenyl-4-yl)methyl]-4,4-diethyl-1H-imidazol-5(4H)-one

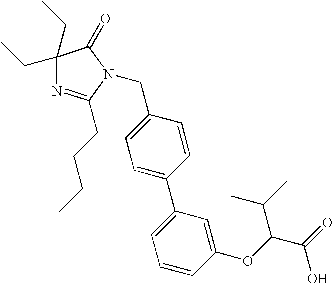

### Compound 64: 2-butyl-1-[(3′-((1-carboxy-1-ethylmethyl)oxy)biphenyl-4-yl)methyl]-4,4-diethyl-1H-imidazol-5(4H)one

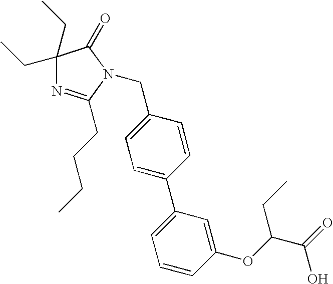

### Compound 65: 2-butyl-1-[(3′-((1-carboxy-1,1-dimethylmethyl)oxy)biphenyl-4-yl)methyl]-4,4-dimethyl-1H-imidazol-5(4H)-one

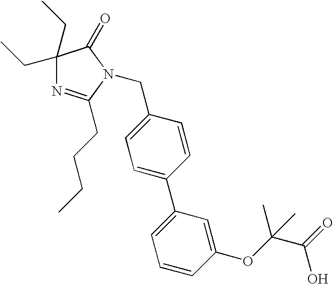

### Compound 66: 2-butyl-1-[(3′-((1-carboxy-1-methylmethyl)oxy)biphenyl-4-yl)methyl]-4,4-diethyl-1H-imidazol-5(4H)one

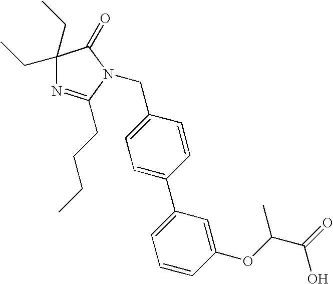

### Compound 67: 2-butyl-1-[(3′-((1-carboxy-1-spirocyclobutylmethyl)oxy)-biphenyl-4-yl)methyl]-4-spirocyclohexyl-1H-imidazol-5(4H)-one

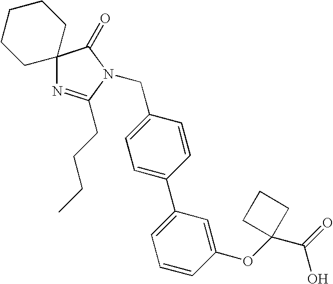

### Compound 68: 2-butyl-1-[(3′-((1-carboxy-1-(1,1-dimethylmethyl)methyl)oxy)-6′-fluoro-biphenyl-4-yl)methyl]-4,4-diethyl-1H-imidazol-5(4H)one

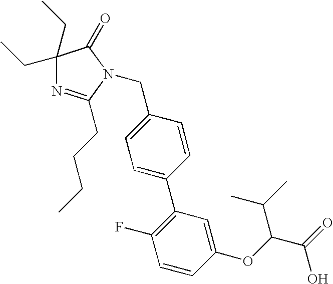

### Compound 69: 2-butyl-1-[(3′-((1-carboxy-1,1-dimethylmethyl)oxy)-6′-fluoro-biphenyl-4-yl)methyl]-4,4-diethyl-1H-imidazol-5(4H)one

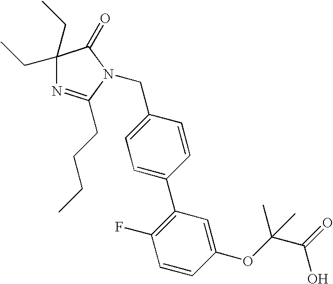

### Compound 70: 2-butyl-1-[(3′-((1-carboxy-1-methylmethyl)oxy)-3-methyl-biphenyl-4-yl)methyl]-4-spirocyclohexyl-1H-imidazol-5(4H)-one

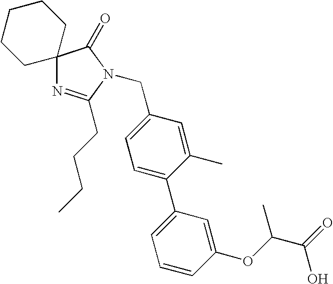

### Compound 71: 2-butyl-1-[(3′-((1-carboxy-1-ethylmethyl)oxy)-3-methyl-biphenyl-4-yl)methyl]-4-spirocyclohexyl-1H-imidazol-5(4H)-one

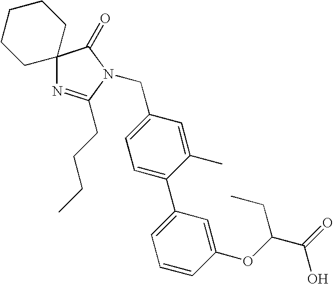

### Compound 72: 2-butyl-1-[(3′-((1-carboxy-1,1-dimethylmethyl)oxy)-3-methyl-biphenyl-4-yl)methyl]-4-spirocyclohexyl-1H-imidazol-5(4H)one

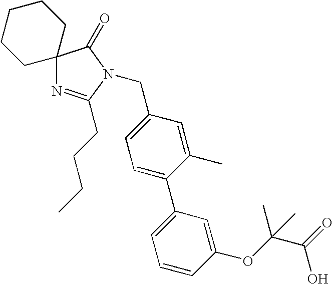

### Compound 73: 2-butyl-1-[(3′-((1-carboxy-1-(1,1-dimethylmethyl)methyl)oxy)-3-methyl-biphenyl-4-yl)methyl]-4-spirocyclohexyl-1H-imidazol-5(4H)one

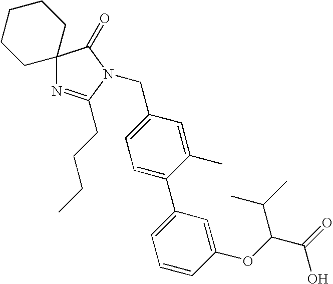

### Compound 74: 2-butyl-1-[(3′-((1-carboxy-1,1-dimethylmethyl)oxy)biphenyl-3-yl)methyl]-4-spirocyclopentyl-1H-imidazol-5(4H)-one

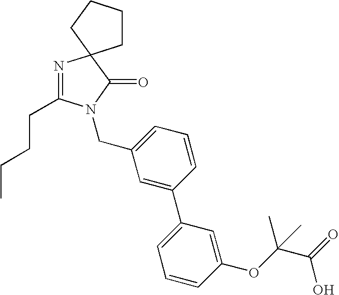

### Compound 75: 2-butyl-1-[(2′-((1-carboxy-1 1-dimethylmethyl)oxy)biphenyl-3-yl)methyl]-4-spirocyclopentyl-1H-imidazol-5(4H)-one

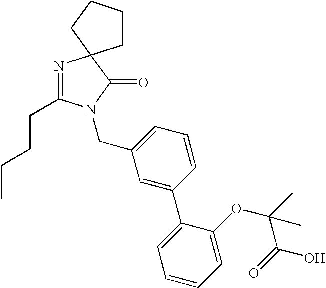

### Compound 76: 2-butyl-1-[(3′-((1-carboxy-1,1-dimethylmethyl)oxy)-6′-propyl-biphenyl-4-yl)methyl]-4-spirocyclopentyl-1H-imidazol-5(4H)-one

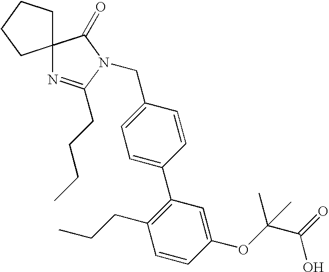

### Compound 77: 2-butyl-1-[(4′-((1-carboxy-1,1-dimethylmethyl)oxy)biphenyl-3-yl)methyl]-4-spirocyclopentyl-1H-imidazol-5(4H)-one

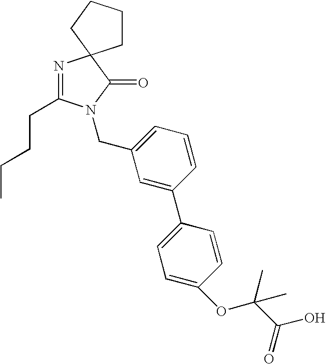

### Compound 78: 2-butyl-1-[(3′-((1-carboxy-1,1-dimethylmethyl)oxy)-2′-fluoro-biphenyl-4-yl)methyl]-4-spirocyclopentyl-1H-imidazol-5(4H)-one

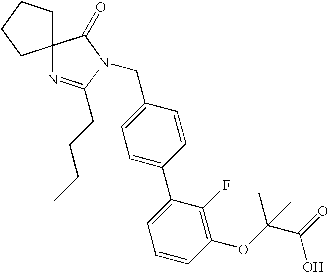

### Compound 79: 2-butyl-1-[(3′-((1-carboxy-1,1-dimethylmethyl)oxy)-4′-methoxy-biphenyl-4-yl)methyl]-4-spirocyclopentyl-1H-imidazol-5(4H)-one

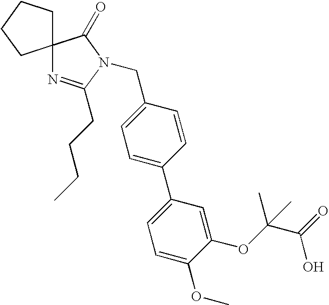

### Compound 80: 2-butyl-1-[(3′-((1-carboxy-1 1-dimethylmethyl)oxy)-6′-ethyl-biphenyl-4-yl)methyl]-4-spirocyclopentyl-1H-imidazol-5(4H)-one

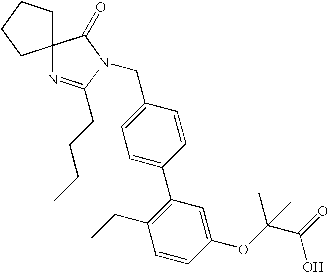

### Compound 81: 2-butyl-1-[(3′-((1-carboxy-1,1-dimethylmethyl)oxy)-4′-isobutyl-biphenyl-4-yl)methyl]-4-spirocyclopentyl-1H-imidazol-5(4H)-one

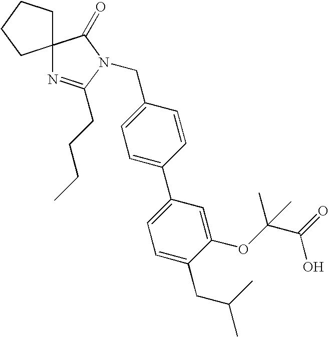

### Compound 82: 2-butyl-1-[(3′-((1-carboxy-1,1-dimethylmethyl)oxy)-2-methoxy-biphenyl-4-yl)methyl]-4-spirocyclopentyl-1H-imidazol-5(4H)-one

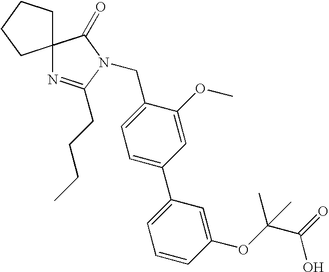

### Compound 83: 2-butyl-1-[(3′-((1-carboxy-1-methylmethyl)oxy)-6′-propyl-biphenyl-4-yl)methyl]-4-spirocyclopentyl-1H-imidazol-5(4H)-one

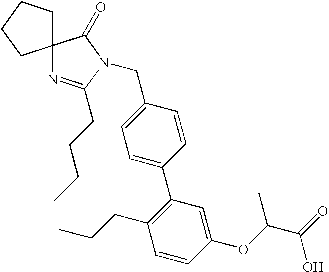

### Compound 84: 2-butyl-1-[(3′-((1-carboxymethyl)oxy)-6′-propyl-biphenyl-4-yl)methyl]-4-spirocyclopentyl-1H-imidazol-5(4H)-one

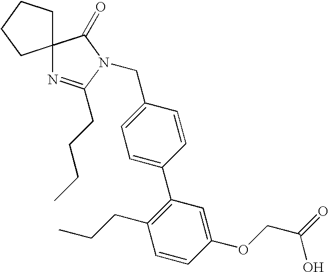

### Compound 85: 2-butyl-1-[(3′-((1-carboxy-1-ethylmethyl)oxy)-6′-propyl-biphenyl-4-yl)methyl]-4-spirocyclopentyl-1H-imidazol-5(4H)-one

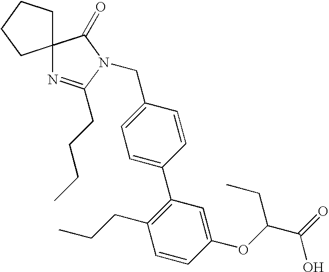

### Compound 86: 2-butyl-1-[(3′-((1-carboxy-1-(1,1-dimethylmethyl)methyl)oxy)-6′-propyl-biphenyl-4-yl)methyl]-4-spirocyclopentyl-1H-imidazol-5(4H)-one

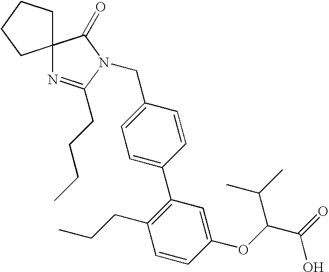

### Compound 87: 2-butyl-1-[(3′-((1-carboxy-1,1-dimethylmethyl)oxy)-6′-isobutyl-biphenyl-4-yl)methyl]-4-spirocyclopentyl-1H-imidazol-5(4H)-one

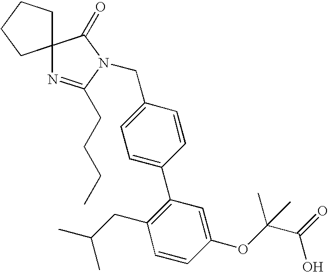

### Compound 88: 2-butyl-1-[(3′-((1-carboxy-1,1-dimethylmethyl)oxy)-3-ethyl-biphenyl-4-yl)methyl]-4-spirocyclopentyl-1H-imidazol-5(4H)-one

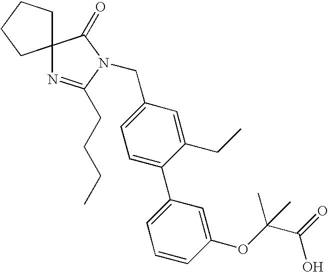

### Compound 89: 2-butyl-1-[(3′-((1-carboxy-1,1-dimethylmethyl)oxy)-6′-cyano-biphenyl-4-yl)methyl]-4-spirocyclopentyl-1H-imidazol-5(4H)-one

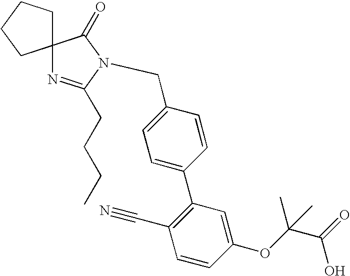

### Compound 90: 2-butyl-1-[(3′-((1-carboxy-1,1-dimethylmethyl)oxy)-3-methoxy-biphenyl-4-yl)methyl]-4-spirocyclopentyl-1H-imidazol-5(4H)-one

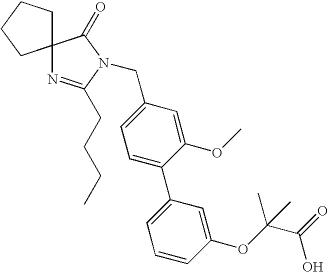

### Compound 91: 2-butyl-1-[(3′-((1-carboxy-1,1-dimethylmethyl)oxy)-2-methyl-biphenyl-4-yl)methyl]-4-spirocyclopentyl-1H-imidazol-5(4H)-one

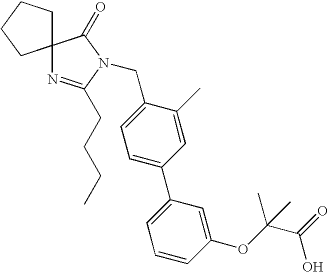

### Compound 92: 2-butyl-1-[[2-[(4-(1-carboxy-1,1-dimethylmethyloxy)phenyl]-6-methyl]thiazolo[3,2-b][1,2,4]triazol-5-yl]methyl]-4-spirocyclopentyl-1H-imidazol-5(4H)-one

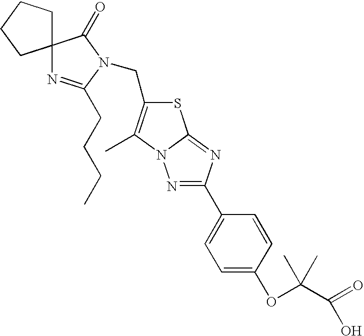

### Compound 93: 2-butyl-1-[[2-[(3-(1-carboxy-1,1-dimethylmethyloxy)phenyl]-6-methyl]thiazolo[3,2-b][1,2,4]triazol-5-yl]methyl]-4-spirocyclopentyl-1H-imidazol-5(4H)-one

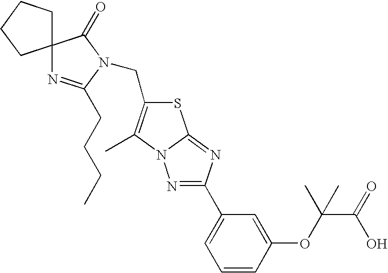

### Compound 94: 2-butyl-1-[(3′-((1-carboxy-1,1-dimethylmethyl)oxy)-3-propyl-biphenyl-4-yl)methyl]-4-spirocyclopentyl-1H-imidazol-5(4H)-one

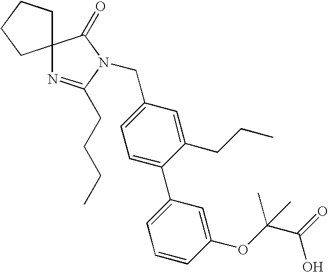

### Compound 95: 2-butyl-1-[(3′-((1-carboxy-1,1-dimethylmethyl)oxy)-4′-nitro-biphenyl-4-yl)methyl]-4-spirocyclopentyl-1H-imidazol-5(4H)-one

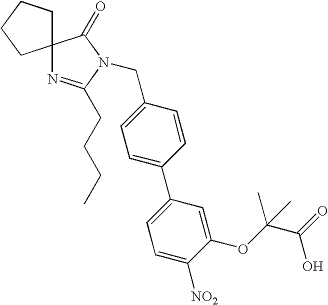

### Compound 96: 2-butyl-1-[(3′-((1-carboxy-1,1-dimethylmethyl)oxy)-3-trifluoromethyl-biphenyl-4-yl)methyl]-4-spirocyclopentyl-1H-imidazol-5(4H)-one

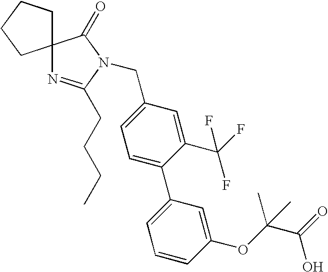

### Compound 97: 2-butyl-1-[(3′-((1-carboxy-1,1-dimethylmethyl)oxy)-3-nitro-biphenyl-4-yl)methyl]-4-spirocyclopentyl-1H-imidazol-5(4H)-one

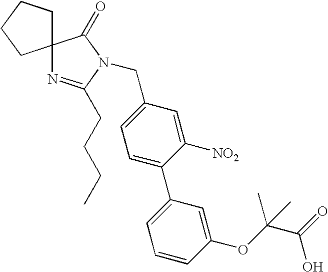

### Compound 98: 2-butyl-1-[(3′-((1-carboxy-1,1-dimethylmethyl)oxy)-4′-propyl-biphenyl-4-yl)methyl]-4-spirocyclopentyl-1H-imidazol-5(4H)-one

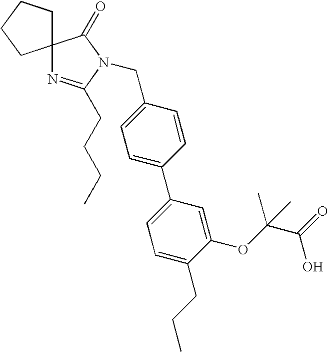

### Compound 99: 2-butyl-1-[(3′-((1-(tetrazol-5-yl)-1-ethylmethyl)oxy)-biphenyl-4-yl)methyl]-4-spirocyclopentyl-1H-imidazol-5(4H)-one

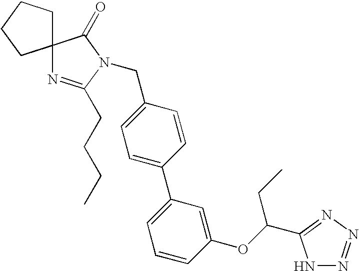

Even more preferably, the compounds according to the invention are:

Compound 1: 2-butyl-1-[(3′-((1-carboxy-1,1-dimethylmethyl)oxy)biphenyl-4-yl)methyl]-4-spirocyclopentyl-1H-imidazol-5(4H)-one;

Compound 12: 1-[(6′-bromo-3′-((1-carboxy-1,1-dimethylmethyl)oxy)biphenyl-4-yl)methyl]-2-butyl-4-spirocyclopentyl-1H-imidazol-5(4H)-one;

Compound 21: 2-butyl-1-[(3′-((1-carboxy-1,1-dimethylmethyl)oxy)biphenyl-4-yl)methyl]-4-spirocyclopentyl-1H-imidazol-5(4H)-one;

Compound 24: 1-[(5′-bromo-2′-((1-carboxy-1,1-dimethylmethyl)oxy)biphenyl-4-yl)methyl]-2-butyl-4-spirocyclohexyl-1H-imidazol-5(4H)-one.

Compound 53: 2-butyl-1-[(3′-((1-carboxy-1-methylmethyl)oxy)biphenyl-4-yl)methyl]-4-spirocyclohexyl-1H-imidazol-5(4H)-one.

Compound 80: 2-butyl-1-[(3′-((1-carboxy-1,1-dimethylmethyl)oxy)-6′-ethyl-biphenyl-4-yl)methyl]-4-spirocyclopentyl-1H-imidazol-5(4H)-one.

The compounds of the present invention include their stereoisomers (diastereoisomers, enantiomers), pure or mixed, racemic forms, their geometric isomers, their tautomers, their salts, their hydrates, their solvates, their solid forms, and mixtures thereof.

The compounds according to the invention can contain one or several asymmetrical centers. The present invention includes stereoisomers (diastereoisomers, enantiomers), pure or mixed, as well as racemic forms.

The present invention also includes geometric isomers of compounds according to the invention.

When an enantiomerically pure (or enriched) mixture is desired, it can be obtained either by purification of the final product or chiral intermediates, or by asymmetrical synthesis following the methods known by one of ordinary skill in the art (for example, using reagents and chiral catalysts). Some compounds according to the invention can have different stable tautomeric forms and all these forms and mixtures thereof are included in the invention.

The present invention also concerns pharmaceutically acceptable salts of compounds according to the invention. Generally, this term designates slightly- or non-toxic salts obtained from organic or inorganic bases or acids. These salts may be obtained during the final purification step of the compound according to the invention or by incorporating the salt into the purified compound.

Some compounds according to the invention and their salts could be stable in several solid forms. The present invention includes all the solid forms of the compounds according to the invention which includes amorphous, polymorphous, mono- and polycrystalline forms.

The compounds according to the invention can exist in non-solvated or solvated form, for example with pharmaceutically acceptable solvents such as water (hydrates) or ethanol.

The present invention also includes the prodrugs of the compounds according to the invention which, after being administered to a subject, turn into compounds such as those described in the invention or into metabolites that present therapeutic effects comparable to the compounds according to the invention. Preferably, the expected metabolites are those metabolites stemming from the oxidation of compounds leading to mono- or poly-hydroxylated compounds or metabolites ensuing from the oxidation of these hydroxylated metabolites (ketonic, hydroxy-ketonic, or carboxylic derivatives). The expected metabolites are also those stemming from glucuronidations or more metabolites ensuing from the opening of the imidazolone cycle or derivatives or other metabolites stemming from N-dealkylation as shown as follows in scheme A:

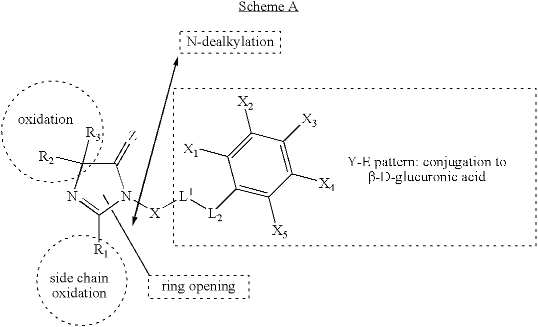

Compounds according to the invention labeled with one or more isotopes are also included in the invention: these compounds are structurally identical but different by the fact that at least one atom of the structure is replaced by an isotope (radioactive or not). Examples of isotopes that can be included in the structure of the compounds according to the invention can be chosen among hydrogen, carbon, nitrogen, oxygen, and sulfur such as ²H, ³H, ¹³C, ¹⁴C, ¹⁵N, ¹⁸O, ¹⁷O, ³⁵S respectively. Radioactive isotopes ³H and ¹⁴C are particularly preferable since they are easy to prepare and detect within the scope of in vivo bioavailability studies of the substances. Heavy isotopes (such as ²H) are particularly preferred for their use as internal standards in analytical studies.

The present invention is also directed to compounds such as above described as medicines.

Another subject-matter of the present invention concerns a pharmaceutical composition comprising, in a pharmaceutically acceptable support, at least one compound as above described, possibly in association with one or several other therapeutic and/or cosmetic active constituents. It is advantageously a pharmaceutical compound for the treatment of pathologies related to lipid and glucid disorders and/or hypertension such as complications associated with metabolic syndrome, diabetes, dyslipidemias, atherosclerosis, cardiovascular diseases, obesity, hypertension, inflammatory diseases (asthma, etc.), insulin resistance, neurodegenerative pathologies, cancers, etc., and/or to diminish the global cardiovascular risk. The pharmaceutical compound according to the invention is preferably used to treat dyslipidemias and/or hypertension (especially hypertension associated or not with dyslipidemias and/or hypertension associated or not with diabetes).

Another subject-matter relates to the use of at least one compound as previously described for the preparation of pharmaceutical compounds intended for treating diverse pathologies, especially those related to metabolic disorders and/or hypertension of which complications associated with metabolic syndrome, diabetes, dyslipidemias, atherosclerosis, cardiovascular diseases, obesity, hypertension, inflammatory diseases (especially asthma, etc.), insulin resistance, neurodegenerative pathologies, cancers, etc., can be cited as examples, as well as for reducing the global cardiovascular risk.

More generally, the subject-matter of the invention concerns the use of at least one compound previously described for the preparation of pharmaceutical compositions intended for treating the cardiovascular disease risk factors related to lipid metabolism disorders and/or hypertension and then, intended for reducing the global risk.

For example (but not limitatively), the molecules according to the invention can advantageously be administered in combination with other therapeutic and/or cosmetic agents, currently available in the market or in development, such as:
anti-diabetics: secretagogues (sulfonylurea (glibenclamide, glimepiride, gliclazide, etc.) and glinides (repaglinide, nateglinide, etc.)), alpha-glucosidase inhibitors, PPARγ agonists (thiazolidinediones such as rosiglitazone, pioglitazone), mixed PPARα/γ agonists (tesaglitazar, muraglitazar), pan-PPARs (compounds that simultaneously activate the 3 PPAR isoforms), biguanides (metformin), Dipeptidyl Peptidase IV inhibitors (MK-431, vildagliptin), Glucagon-Like Peptide-1 (GLP-1) agonists (exenatide), etc.
insulin
lipid-lowering and/or cholesterol-lowering molecules: fibrates (fenofibrate, gemfibrozil), HMG CoA reductase inhibitors or hydroxylmethylglutaryl coenzyme A reductase (statins such as atorvastatin, simvastatin, fluvastatin), cholesterol absorption inhibitors (ezetimibe, phytosterols), CETP or cholesteryl ester transfer protein inhibitors (torcetrapib), ACAT or acyl-coenzyme a cholesterol acyltransferase (avasimibe, eflucimibe), MTP (Microsomal Triglyceride Transfer Protein) inhibitors, biliary acid sequestering agents (cholestyramine), vitamin E, polyunsaturated fatty acids, omega 3 fatty acids, nicotinic acid type derivatives (niacin), etc.
anti-hypertensive agents and hypotensive agents: ACE (Angiotensin-Converting Enzyme) inhibitors (captopril, enalapril, ramipril, or quinapril), angiotensin II receptor antagonists (losartan, valsartan, telmisartan, eposartan, irbesartan, etc.), beta blockers (atenolol, metoprolol, labetalol, propranolol), thiazide and non-thiazide diuretics (furosemide, indapamide, hydrochlorthiazide, anti-aldosterone), vasodilators, calcium channel blockers (nifedipine, felodipine, or amlodipine, diltiazem or verapamil), etc.
anti-platelet agents: Aspirin, Ticlopidine, Dipyridamol, Clopidogrel, flurbiprofen, etc.
anti-obesity agents: Sibutramine, lipase inhibitors (orlistat), PPAR (Peroxisome proliferator-activated receptor)δ agonists and antagonists, cannabinoid CB1 receptor antagonists (especially rimonabant), etc.
anti-inflammatory agents: for example, corticoids (prednisone, betamethasone, dexamethasone, prednisolone, methylprednisolone, hydrocortisone, etc.), NSAIDs or non-steroidal anti-inflammatory drugs derived from indole (indomethacin, sulindac), NSAIDs of the arylcarboxylic group (tiaprofenic acid, diclofenac, etodolac, flurbiprofen, ibuprofen, ketoprofen, naproxen, nabumetone, alminoprofen), NSAIDs derived from oxicam (meloxicam, piroxicam, tenoxicam), NSAIDs from the fenamate group, COX2 selective inhibitors (celecoxib, rofecoxib), etc.
antioxidant agents: for example probucol, etc.
agents used in the treatment of cardiac insufficiency: thiazidic and non-thiazidic diuretics (furosemide, indapamide, hydrochlorthiazide, antialdosterone), ACE (Angiotensin converting enzyme) inhibitors (captopril, enalapril, ramipril or quinapril), digitalis drugs (digoxin, digitoxin), beta blockers (atenolol, metoprolol, labetalol, propranolol), phosphodiesterase inhibitors (enoximone, milrinone), etc.
agents used in the treatment of coronary insufficiency: beta blockers (atenolol, metoprolol, labetalol, propranolol), calcium channel blockers (nifedipine, felodipine, or amlodipine, bepridil, diltiazem or verapamil), NO (nitric oxide) donors (trinitrine, isosorbide dinitrate, molsidomine), amiodarone, etc.
anti-cancer drugs: cytotoxic agents (agents interacting with DNA (Deoxyribonucleic Acid), alkylating agents, cisplatin, and derivatives), cytostatic agents (GnRH (Gonatropin-Releasing Hormone) analogues, somatostatin analogues, progestin, anti-oestrogen drugs, aromatase inhibitors, etc.), immune response modulators (interferons, IL2, etc.), etc.
antiasthmatic drugs such as bronchodilators (especially beta 2 receptor agonists), corticoids, cromoglycate, leucotriene receptor antagonists (especially montelukast), etc.
corticoids used in the treatment of skin pathologies such as psoriasis and dermatitis
vasodilators and/or anti-ischemic agents (especially buflomedil, ginkgo biloba extract, naftidrofuryl, pentoxifylline, piribedil), etc.

The invention also concerns a method for treating pathologies related to lipid metabolism and/or hypertension comprising the administration to a subject, in particular a human, of an effective quantity of a compound or a pharmaceutical composition as above-defined. Within the context of the invention, the term “an effective quantity” refers to an amount of the compound, sufficient to produce the desired biological result. Within the context of the invention, the term “subject” means a mammal and more particularly a human.

The term “treatment” designates curative, symptomatic, or preventative treatment. The compounds of the present invention can thus be used upon subjects (such as mammals, in particular humans) having a declared disease. The compounds of the present invention can also be used to delay or slow down the progress or prevent the further progress of the disease, thus improving the subjects' condition. The compounds of the present invention can finally be administered to healthy subjects that might normally develop the disease or have a significant risk of developing the disease.

Pharmaceutical inventions according to the invention advantageously include one or several excipients or vehicles, acceptable within a pharmaceutical context. (e.g. saline solutions, physiological solutions, isotonic solutions, etc., compatible with pharmaceutical usage and well-known by one of ordinary skill in the art. The compositions can contain one or several agents or vehicles chosen among dispersants, solubilizers, stabilizers, preservatives, etc. Usable agents or vehicles for these formulations (liquid and/or injectable and/or solid) are notably methylcellulose, hydroxymethylcellulose, carboxymethylcellulose, polysorbate 80, mannitol, gelatin, lactose, vegetable oils, acacia, liposomes, etc. The compositions can be formulated in the form of injectable suspensions, gels, oils, pills, suppositories, powders, gelcaps, capsules, aerosols, etc., eventually by means of galenic forms or devices assuring a prolonged and/or slow release. For this kind of formulation, agents such as cellulose, carbonates, or starches can advantageously be used.

The compounds or compositions according to the invention can be administered in different ways and in different forms. Thus, for example, they may be administered in a systematic way, per os, parenterally, by inhalation, or by injection, such as for example intravenously, by intramuscular route, by subcutaneous route, by transdermal route, by intra-arterial route, etc. For the injections, the compounds are generally conditioned in the form of liquid suspensions which can be injected using syringes or perfusions, for example.

It is understood that the speed and/or the dose relative to the injection can be adapted by one of ordinary skill in the art, in function of the patient, the pathology, the form of administration, etc. Typically, the compounds are administered in doses varying between 1 μg and 2 g per administration, preferably from 0.1 mg to 1 g per administration. Administrations can be performed daily or several times per day. Additionally, the compositions according to the invention can include, moreover, other agents or active constituents.

Another subject-matter of the invention concerns the processes for preparing the compounds derived from polysubstituted imidazolones according to the invention.

The compounds according to the invention can be prepared using commercially available products to create a combination of chemical reactions well-known to one of ordinary skill in the art.

The subject-matter of the present invention concerns a process for the preparation of above disclosed compounds according to the invention, comprising:
(i) a step of condensation of a halogenated derivative or of a derivative carrying a sulfonate group, preferably mesyl or tosyl, on a mono- or poly-substituted heterocycle such as imidazolone,
   or a step of cyclisation of an appropriately substituted amino amide and a ortho-ester, and possibly, before and/or after step (i)
(ii) one or several steps of insertion and/or transformation of functional groups.

### Preparation of Compounds of Formula (I)

Preferably, the compounds of general formula (I) are synthesized using hydrolysis, thermolysis, or hydrogenolysis (A) of an intermediate of general formula (Ia):

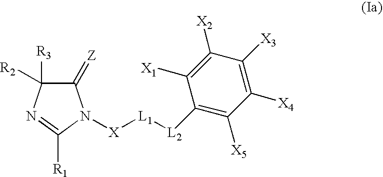

in which R1, R2, R3, Z, X, L1, L2, X1, X2, X3, X4, and X5 are as previously defined, with at least one of groups X1, X2, X3, X4, and X5 representing a type —Y— E′ group, the E′ group being by definition a group that can be used to afford the E group via hydrolysis, thermolysis, or hydrogenolysis.

This synthetic route is preferably applied if E contains at least one carboxylic acid function. In such a case, E′ is a group comprising a chemical function which can be transformed into a carboxylic derivative via hydrolysis, thermolysis, or hydrogenolysis.

Some examples of chemical functions which are hydrolysable in carboxylic acid are acid derivatives (esters, thioesters, orthoesters, etc.) and nitrile, tetrazolyl, 1,3-oxazol-2-yl, 1,3-oxazolin-2-yl, etc.

The hydrolysis reactions can be advantageously performed in the presence of an organic acid (e.g. trifluoroacetic acid) or an inorganic acid (e.g. hydrochloric acid) or in the presence of a base (e.g. sodium hydroxide) in water or a mixture of solvents containing water (water/methanol, water/ethanol, water/THF (tetrahydrofuran), water/dioxane, etc.) They are carried out at temperatures between −10° C. and 120° C., preferably between 20° C. and the temperature of the solvent reflux.

Some examples of chemical functions the thermolysis generates an acid function are tertiary alkyl esters, preferably tertiobutyl esters.

The thermolysis reactions are preferably carried out in absence of solvent (melt blend) or in an inert solvent such as dichloromethane, chloroform, toluene, tetrahydrofuran, or dioxane. Adding catalytic amounts of strong acids, such as paratoluenesulfonic acid, is generally necessary for thermolysis. These reactions are preferably performed using heating, advantageously at the boiling temperature of the used solvent.

Some examples of chemical functions the hydrogenolysis generates an acid function are arylalkyl esters, preferably benzyl esters.

The hydrogenolysis reactions are carried out in the presence of a metallic catalyst (Pd/C, Pt, etc.) in a suitable solvent such as methanol, ethanol, tetrahydrofuran (THF), acetic acid, ethyl acetate, etc. They are carried out at temperatures between 0° C. and 60° C., preferably at room temperature, under hydrogen pressure between 1 and 6 bars. An alternative route uses ammonium formate to produce hydrogen in situ.

Preferably, E′ contains acid function(s) in a protected form. It is up to the one of ordinary skill in the art to choose the most appropriate protection group in function of the different substituents.

In accordance with a preferred embodiment of the invention, the compounds (Ia) corresponding to the esterified form of the compounds (I).

According to the nature of the ester function(s) within the E′ group, different methods are applicable for regenerating E acid:
(i) basic hydrolysis: this methodology is applicable to alkyl esters such as methyl ester and ethyl ester;
(ii) acid hydrolysis: this methodology is applicable to alkyl esters such as tert-butyl esters;
(iii) hydrogenolysis: this methodology is applicable to benzyl type esters and analogues.

If E contains at least one tetrazolyl function, then E′ can be a group containing a chemical function such as a nitrile function, which can be transformed into tetrazole by methods well-known to the one of ordinary skill in the art, or a tetrazole group protected by a protecting group, preferably a benzyloxymethylether or trityl group which may be hydrolyzed in accordance with methods that are well-known to the one of ordinary skill in the art.

If E contains at least one amide function, then E′ is a group containing a chemical function which can be transformed into amide, such as a carboxylic acid function, via methods well-known to one of ordinary skill in the art.

If E contains at least one acylsulfonamide function, then E′ is a group containing a chemical function which can be transformed into acylsulfonamide, such as a carboxylic acid function, by methods well-known to one of ordinary skill in the art.

If E contains at least one hydrazide function, then E′ is a group containing a chemical function which can be transformed into hydrazide, such as a carboxylic acid function, by methods well-known to one of ordinary skill in the art.

The compounds of general formula (I) according to the invention, in which Z represents a sulfur atom, can be obtained from compounds of general formula (Ia) according to the invention in which Z represents an oxygen atom by reaction with classical reagents well-known to one of ordinary skill in the art, for example using Lawesson's reagent.

### Preparation of Compounds of Formula (Ia)

The compounds of formula (Ia) in which R1, R2, R3, Z, X, L1, L2, X1, X2, X3, X4, and X5 are as previously defined, with at least one of groups X1, X2, X3, X4, and X5 representing a —Y-E′ type group, the E′ group being by definition a group that can be used to produce the E group via hydrolysis, thermolysis, or hydrogenolysis, can also be obtained according to the following process (scheme 1):

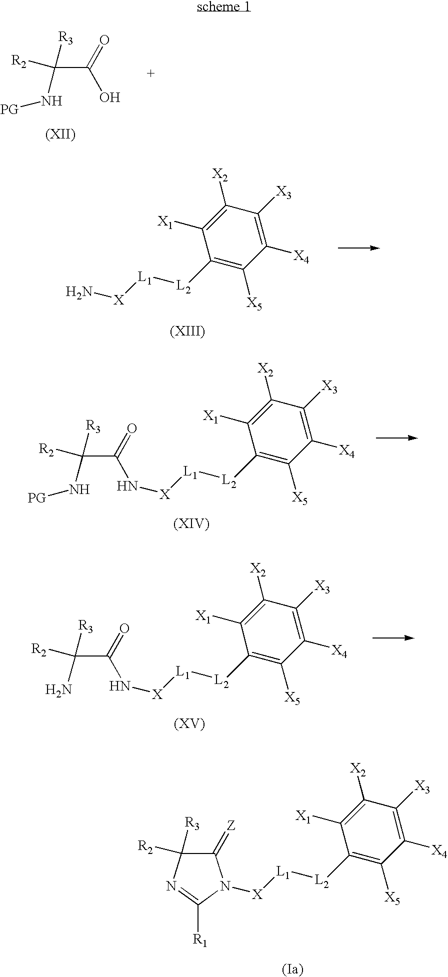

(a) reaction of an amino acid of formula (XII) in which PG represents a protecting group such as BOC (tert-butyloxycarbonyl) or Cbz (benzyloxycarbonyl) with a compound of formula (XIII) in presence of coupling reagents or activators well-known to one of ordinary skill in the art to obtain a compound of formula (XIV)
(b) deprotection of the compound of formula (XIV) in conditions well-known to one of ordinary skill in the art to obtain a compound of formula (XV)
(c) reaction of a compound of formula (XV) with an ortho ester of R1C formula (OR9)₃, in which R1 is as previously defined and R9 represents a short alkyl chain (C1-C4).

The compounds of formula (Ia) in which R1, R2, R3, Z, X, L1, L2, X1, X2, X3, X4, and X5 are such as previously defined with at least one of the groups X1, X2, X3, X4, and X5 representing a Y-E′ group, group E′ being by definition a group which by hydrolysis, thermolysis, or hydrogenolysis leads to the E group, can be obtained preferably and advantageously according to the following process (see scheme 2) by condensation between a heterocyclic derivative of general formula (XVI), in which R1, R2, R3, and Z are such as previously defined, and a derivative of general formula (XVII) in which X, L1, L2, X1, X2, X3, X4, and X5 are such as previously defined, W representing a carboxyl group or a derivative, such as ester (—COOR4), thioester (—COSR4), amide (—CONR4R5), thioamide (—CSNR4R5), or an acylsulfonamide (—CONHSO₂R6) group, and LG representing a leaving group chosen, for example, from among halogens (iodine, bromine, chlorine) or a group such as mesyl or tosyl in the presence of probable activators well-known to the one of ordinary skill in the art.

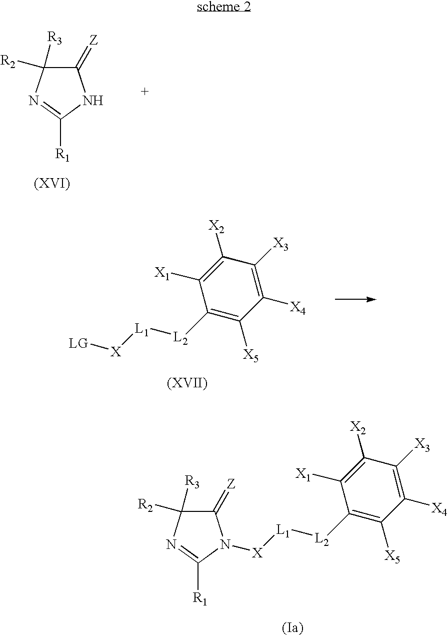

The condensation reaction can be achieved in multiple ways, well-known to the one of ordinary skill in the art. The preferred way consists in working with a solvent such as dichloromethane, chloroform, diethyl ether, tetrahydrofuran, acetonitrile, or dimethylformamide. Such reactions are performed in the presence of bases like sodium hydride or carbonates (as potassium carbonate or sodium carbonate). These reactions can be performed at temperatures between −25° C. and 250° C., preferably between −10° C. and the boiling point of the solvent.

The compounds of general formula (Ia) in which X, L1, L2, X1, X2, X3, X4, and X5 are as previously defined, at least one of groups X1, X2, X3, X4, or X5 being a type Y-E′ group, can be obtained preferably and advantageously according to the following process (see scheme 3) by reaction of a compound of formula LG-E′ with a compound of formula (Ib) in which X, L1, L2, X1, X2, X3, X4, and X5 are as previously defined, at least one of the X1, X2, X3, X4, or X5 groups being a Y—H type group, Y representing an oxygen atom or a sulfur atom (scheme 3). E′ is by definition a group which, by hydrolysis, thermolysis, or hydrogenolysis, lead to the formation of group E; and LG represents a leaving group chosen, for example, from among the halogens (iodine, bromine, chlorine) or a sulfonate type leaving group, such as mesylate or toyslate, possibly in the presence of activators well-known to the one of ordinary skill in the art.

The condensation reaction of the LG-E′ group can be achieved in multiple ways, well-known to the one of ordinary skill in the art. The preferred way consists in working with a solvent such as dichloromethane, chloroform, diethyl ether, tetrahydrofuran, acetonitrile, or dimethylformamide. Such reactions are performed in the presence of bases such as sodium hydride or carbonates (e.g. potassium carbonate or sodium carbonate). These reactions can be performed at temperatures between −25° C. and 250° C., preferably between −10° C. and the boiling point of the solvent.

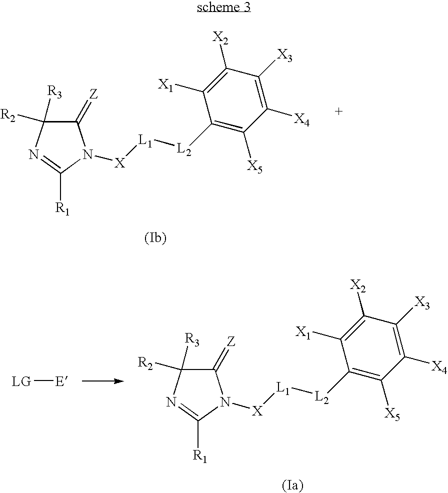

### Preparation of Compounds of Formula (XVI)

The compounds of general formula (XVI) in which R1, R2, and R3 are as previously defined and in which Z represents an oxygen atom, are prepared using an amino acid ester of general formula (XVIII) and an imidate of general formula (XIX) in which R1, R2, and R3 are as previously defined and R0 represents an alkyl group, preferably methyl or ethyl, in accordance to a procedure described by Bernhart C et al., 1993 (scheme 4).

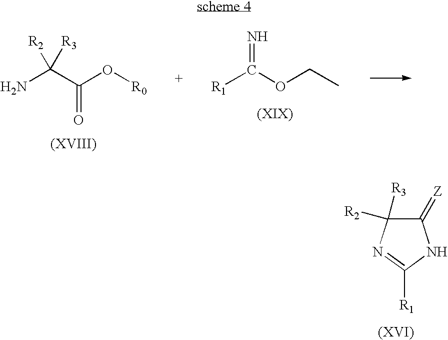

The compounds of general formula (XVIII) are well-known, commercially available or can be prepared in accordance with methods well-known to one of ordinary skill in the art, for example, using compounds of formula (XVIII) in which R2 and R3 are as previously defined and R0 represents a hydrogen atom in accordance with the Fischer esterification method (Tsang J W et al., 1984). These compounds can also be obtained optically pure using asymmetrical synthesis methods or chiral purification methods well-known to one of ordinary skill in the art.

The compounds of general formula (XIX) are prepared using a nitrile of formula (XX) in ethanol in the presence of an acid as hydrochloric acid, R1 being as previously defined (Bernhart C et al., 2003, McElwain S and Nelson J, 1942) (scheme 5).

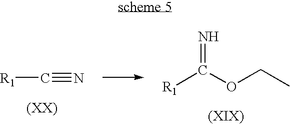

The compounds of general formula (XX) are well-known, commercially available or can be prepared in accordance with methods well-known to one of ordinary skill in the art.

According to another synthetic process, the compounds of general formula (XVI) in which R1, R2, and R3 are as previously defined and in which Z represents an oxygen atom, are prepared using an amino-amide of general formula (XXI) and an alkyl orthoester of general formula (XXII) in which R1, R2, and R3 are as previously defined and R′0 represents a short alkyl chain (C1-C4), in an acid medium according to a process well-known to one of ordinary skill in the art (Bernhart C, Perreaut P, Ferrari B, Muneaux Y, Assens J, Clement J, Haudricourt F, Muneaux C, Taillades J, and Vignal M, 1993) (scheme 6).

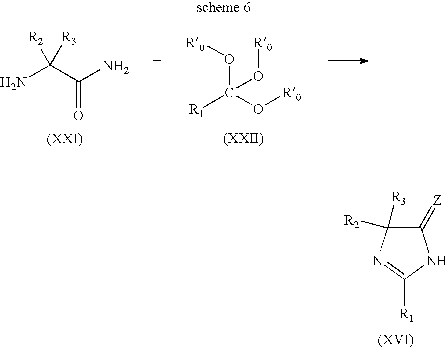

The compounds of general formula (XXI) and (XXII) in which R′0, R1, R2, and R3 are as previously defined, are well-known, commercially available or can be prepared in accordance with methods well-known to one of ordinary skill in the art.

According to a third synthetic process, the compounds of general formula (XVI) in which R1, R2, and R3 are as previously defined and Z represents an oxygen atom, are prepared by reaction of an acid halide of general formula (XXIII) in which R1 is as previously defined and Hal represents a halogen, preferably a chlorine atom, with an amino-amide of general formula (XXI) in which R2 and R3 are as previously defined (scheme 7).

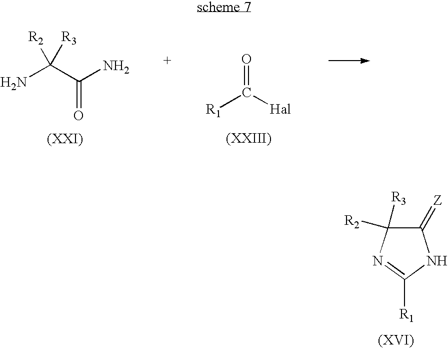

The compounds of general formula (XXI) and (XXIII) in which R1, R2, and R3 are as previously defined, are well-known, commercially available or can be prepared in accordance with methods well-known to one of ordinary skill in the art.

### Preparation of Compounds of Formula (XVII)

The compounds of general formula (XVII) in which X, L1, L2, X1, X2, X3, X4, and X5 are as previously described, at least one of groups X1, X2, X3, X4, or X5 being a Y-E′ type group, and in which LG represents a leaving group such as halogen, advantageously a bromine atom or a chlorine atom, or a sulfonate, advantageously a mesylate or a tosylate, are prepared under classical conditions well-known to one of ordinary skill in the art (scheme 8), using compounds of general formula (XXIV) in which X, L1, L2, X1, X2, X3, X4, and X5 are as previously defined, with at least one of groups X1, X2, X3, X4, or X5 being a Y-E′ type group. In the particular case in which X represents a methylene group and L1 represents a heterocycle or a cycle as previously defined, the synthesis of compounds of general formula (XVII) in which LG represents a halogen atom, preferably a bromine atom, can be advantageously carried out by free-radical halogenations under conditions well-known to one of ordinary skill in the art.

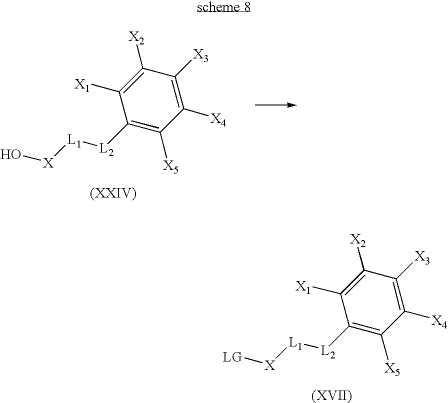

The compounds of general formula (XXIV) in which X, L1, L2, X1, X2, X3, X4, and X5 are as previously defined, with at least one of groups X1, X2, X3, X4, or X5 being a Y-E′ type group, are obtained by reaction of a compound of formula LG-E′ with a compound of formula (XXIVa) in which X, L1, L2, X1, X2, X3, X4, and X5 are as previously defined, with at least one of groups X1, X2, X3, X4, or X5 being a Y—H type group, Y representing an oxygen atom or a sulfur atom (scheme 9). E′ is by definition a group which, by hydrolysis, thermolysis, or hydrogenolysis, leads to the formation of the group E; and LG represents a leaving group chosen, for example, from among the halogens (iodine, bromine, chlorine) or a sulfonate type group, such as mesylate or tosylate, possibly in the presence of activators well-known to the one of ordinary skill in the art.

The condensation reaction of the LG-E′ group can be achieved in multiple ways, well-known to the one of ordinary skill in the art. The preferred way consists in working with a solvent such as dichloromethane, chloroform, diethyl ether, tetrahydrofuran, acetonitrile, or dimethylformamide. Such reactions are performed in the presence of bases as sodium hydride or carbonates (like potassium carbonate or sodium carbonate). These reactions can be performed at temperatures between −25° C. and 250° C., preferably between −10° C. and the boiling point of the solvent.

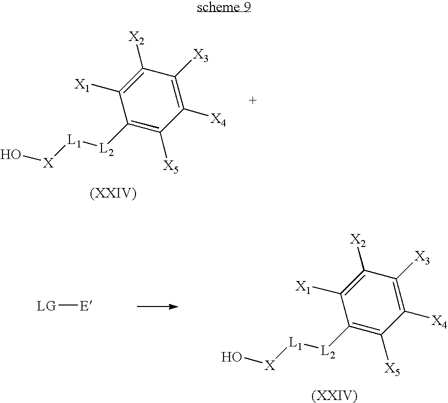

The compounds of Formula (XXIVa), in which X, L1, L2, X1, X2, X3, X4, and X5 are as previously defined, with at least one of groups X1, X2, X3, X4, or X5 being a Y—H type group in which Y represents an oxygen or sulfur atom, are well-known, commercially available or can be prepared in accordance to methods well-known to one of ordinary skill in the art.

Some synthetic routes are preferred for the synthesis of compounds of formula (XVII):
the routes include especially applying the Suzuki reaction (Zou Y et al., 2004) for compounds of general formula (XVII) in which L2 represents a covalent bond and L1 represents a formula (II) group in which X′1, X′2, X′3, X′4, and X′5, are as previously defined
the routes include especially applying aromatic nucleophilic substitution reactions (Sawyer J S et al., 1998) for compounds of formula (XVII) in which L2 represents an oxygen atom or a sulfur atom and L1 represents a formula (II) group in which X′1, X′2, X′3, X′4, and X′5, are as previously described.
the routes include especially applying the Friedel-Crafts reaction to obtain compounds of formula (XVII) in which L2 represents a carbonyl group and L1 represents a formula (II) group in which X′1, X′2, X′3, X′4, and X′5, are as previously described.

The preferred routes for synthesis include especially applying a selective reduction reaction of compounds of formula (XVII) in which L2 represents a carbonyl group and L1 represents a formula (II) group in which X′1, X′2, X′3, X′4, and X′5 are as previously defined so as to obtain compounds of formula (XVII) in which L2 represents a methylene group and L1 represents a formula (II) group in which X′1, X′2, X′3, X′4, and X′5, are as previously defined.

### Preparation of Compounds of Formula (Ib)

The compounds of general formula (Ib) in which X, X1, X2, X3, X4, and X5 are as previously defined, with at least one of groups X1, X2, X3, X4, or X5 being a OR4 group, advantageously a hydroxy or methoxy group, L2 represents a covalent bond and L1 represents a group of formula (II) in which X′1, X′2, X′3, X′4, and X′5 are as previously defined, can be obtained preferably and advantageously according to the following process (see scheme 10) using Suzuki type coupling reaction catalysed with palladium of a derivative of general formula (XXV) in which X6, X7, X8, X9, and X10, identical or different, represent independently a hydrogen or halogen atom, an acid or boronic ester group, a NO₂, nitrile, alkyl, cycloalkyl, alkenyl, alkynyl, aryl, arylalkyl, —OR4, —SR4, —NR4R5, —SOR6, —SO₂R6 group, a heterocycle, with one of X6, X7, X8, X9, and X10 being an RG reactive group such as a halogen, a boronic acid, or a boronic ester and an aromatic derivative of general formula (XXVI) in which X1, X2, X3, X4, and X5, are as previously defined, L2 being a covalent bond and RG being a reactive group such as a halogen or a boronic acid in the presence of metallic catalysts well-known to one of ordinary skill in the art.

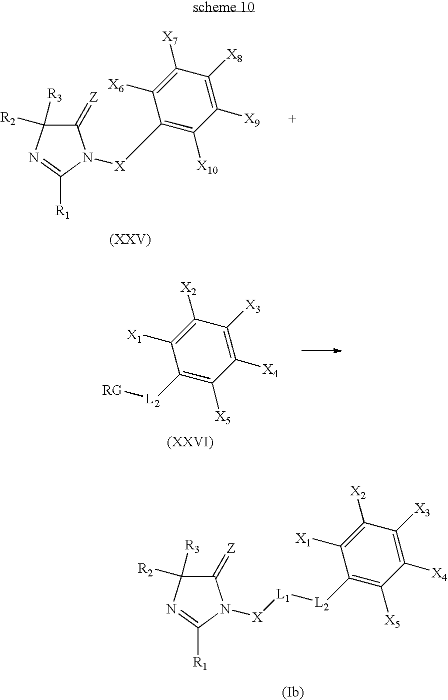

The compounds of general formula (Ib) in which L1, L2, X, X1, X2, X3, X4, and X5 are as previously defined, with at least one of groups X1, X2, X3, X4, or X5 being a hydroxy type OR4 group, can be obtained preferably and advantageously by a demethylation reaction of compounds of general formula (Ib) in which L1, L2, X, X1, X2, X3, X4, and X5 are as previously defined, with at least one of groups X1, X2, X3, X4, or X5 being a OR4 group of methoxy type under conditions well-known to one of ordinary skill in the art, for example in the presence of boron tribromide.

### Preparation of Compounds of Formula (XXV)

The compounds of general formula (XXV) in which X6, X7, X8, X9, and X10, identical or different, represent independently a hydrogen or halogen atom, an acid or boronic ester group, a NO₂, nitrile, alkyl, cycloalkyl, alkenyl, alkynyl, aryl, arylalkyl, —OR4, —SR4, —NR4R5, —SOR6, —SO₂R6 group, a heterocycle, one of X6, X7, X8, X9, and X10 being a reactive group of halogen type, boronic acid type or boronic ester type, can be obtained preferably and advantageously according to the following process (see scheme 11) of condensation between a heterocyclic derivative of general formula (XVI), in which R1, R2, R3, and Z are as previously defined, and a derivative of general formula (XXVII) in which X, X6, X7, X8, X9, and X10 are as previously defined, and with LG representing a leaving group chosen, for example, from among the halogens (iodine, bromine, chlorine) or a sulfonate group such as mesyl or tosyl, possibly in the presence of activators well-known to one of ordinary skill in the art.

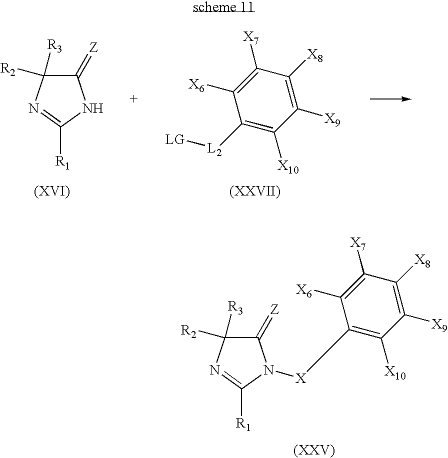

The condensation reaction can be achieved in multiple ways, well-known to the one of ordinary skill in the art. The preferred way consists in working with a solvent such as dichloromethane, chloroform, diethyl ether, tetrahydrofuran, acetonitrile, or dimethylformamide. Such reactions are performed in the presence of bases as sodium hydride or carbonates (as potassium carbonate or sodium carbonate). These reactions can be performed at temperatures between −25° C. and 250° C., preferably between −10° C. and the boiling point of the solvent.

The compounds of general formula (XXVI) and (XXVII) in which L2, X, X1, X2, X3, X4, and X5 are as previously defined and X6, X7, X8, X9, and X10, identical or different, represent independently a hydrogen or halogen atom, an acid or boronic ester group, a NO₂, nitrile, alkyl, cycloalkyl, alkenyl, alkynyl, aryl, arylalkyl, —OR4, —SR4, —NR4R5, —SOR6, —SO₂R6 group, a heterocycle, with one of X6, X7, X8, X9, and X10 being an RG reactive group such as a halogen, a boronic acid, or a boronic ester are well-known compounds, commercially available or can be prepared in accordance with methods well-known to one of ordinary skill in the art.

### FIGURE LEGEND

### Abbreviations:

Cpd=compounds

Ctrl=control

Ang. II=angiotensin II

P=arterial pressure

mpk=mg/kg/day

LDL-cholesterol=Low Density Lipoprotein cholesterol

HDL-cholesterol=High Density Lipoprotein cholesterol

VLDL-cholesterol=Very Low Density Lipoprotein cholesterol

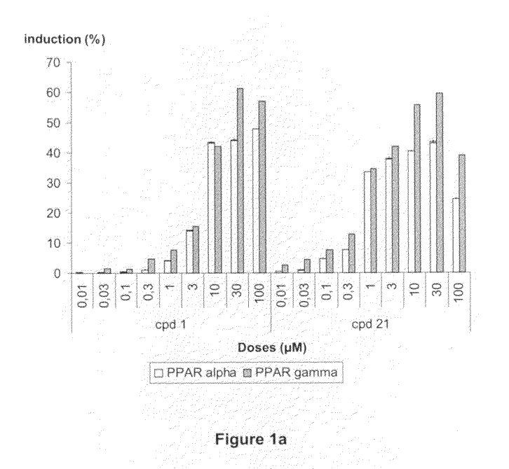

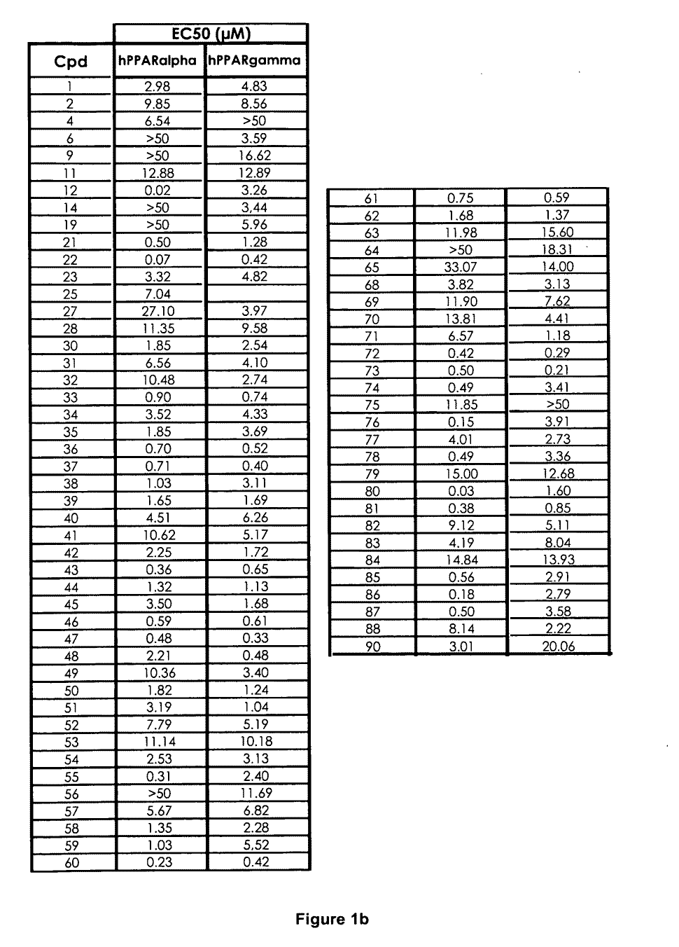

### FIG. 1: In Vitro Evaluation of PPAR Activating Properties of the Compounds according to the Invention

The activation of PPARs is evaluated in vitro using a monkey kidney fibroblast line (COS-7), by measuring the transcriptional activity of chimeras made up of the DNA binding domain of the Gal4 transcription factor of yeast and of the binding domain to the ligand of the different PPARs.

The compounds are tested at doses of between 0.01 and 100 μM on Gal4-PPARα, γ, and δ chimeras. The induction factor, i.e. the ratio between the luminescence induced by the compound and the luminescence induced by the control, is measured for each condition. The higher the induction factor is, the more the compound has PPAR activating properties.

FIG. 1a: The compounds according to the invention were tested at doses between 0.01 and 100 μM on Gal4-PPARα and Gal4-PPARγ chimeras

FIG. 1b: EC50 (μM) relative to PPARα and PPARγ (human isoforms) activating properties of compounds according to the invention. EC50 corresponds to the compound concentration for which 50% of the maximum effect is obtained. The lower the EC50 is, the higher the affinity of the compound of the invention for the receptor.

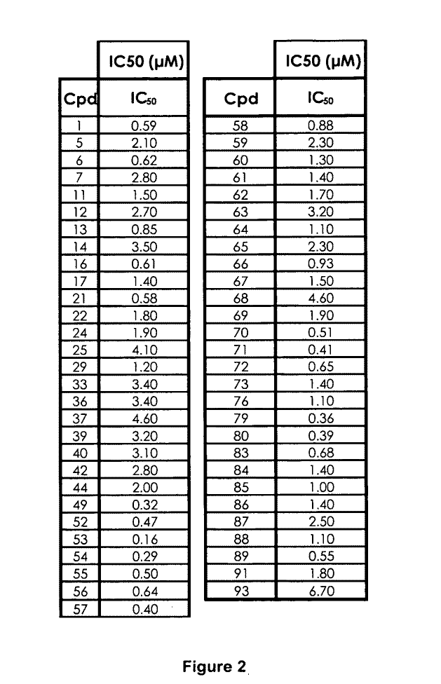

FIG. 2: In Vitro Evaluation of the Bond Between the Compounds According to the Invention and the Human Angiotensin II AT1 Receptor

The disclosed results reflect the specific bond of the compounds according to the invention to the human angiotensin II AT1 receptor. The specific bond corresponds to the difference between the total bond and the non-specific bond determined in the presence of an excess of non-labeled reference ligand (saralasin). The displacement of the radio-labeled molecule was measured for each dose of compound according to the invention. IC50 stands for the compound concentration needed to inhibit 50% of the binding of the reference molecule (saralasine). The lower the IC50 is, the stronger the affinity of the compound for AT1 receptor.

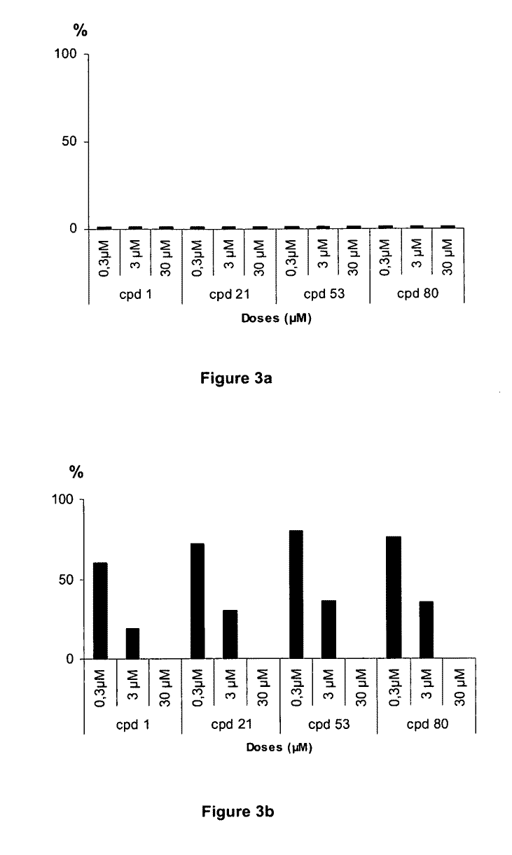

FIGS. 3a and 3b: Ex Vivo Evaluation of the Antagonist Effect of the Compounds According to the Invention on the Angiotensin II AT1 Receptor

The disclosed results, expressed in percentages, show the effects of compounds 1, 21, 53 and 80 according to the invention tested as agonists or antagonists of human angiotensin II AT1 receptor on rabbit thoracic aorta. The parameter measured was the maximum change in tension induced by each concentration of compound. The results were expressed in percentages of the control response to angiotensin II.

FIG. 3a: Agonist activity of compounds according to the invention at 0.3, 3, and 30 μM.

FIG. 3b: Antagonist activity of compounds according to the invention at 0.3, 3, and 30 μM.

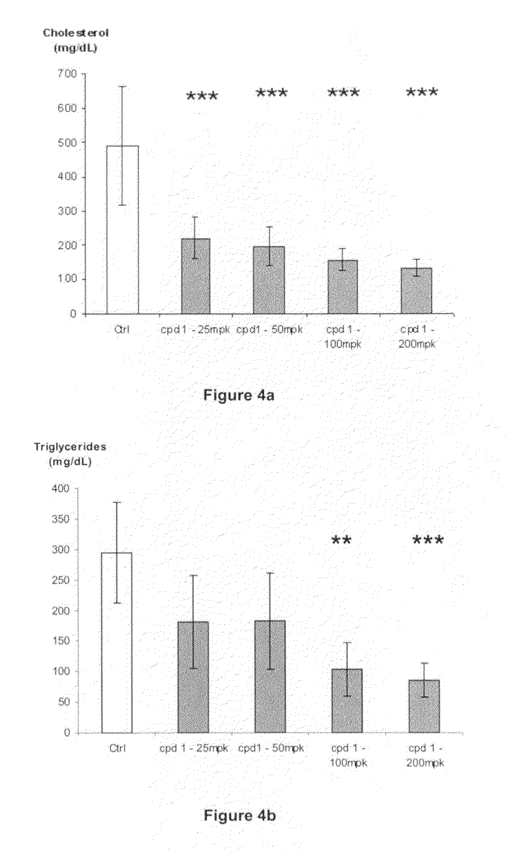

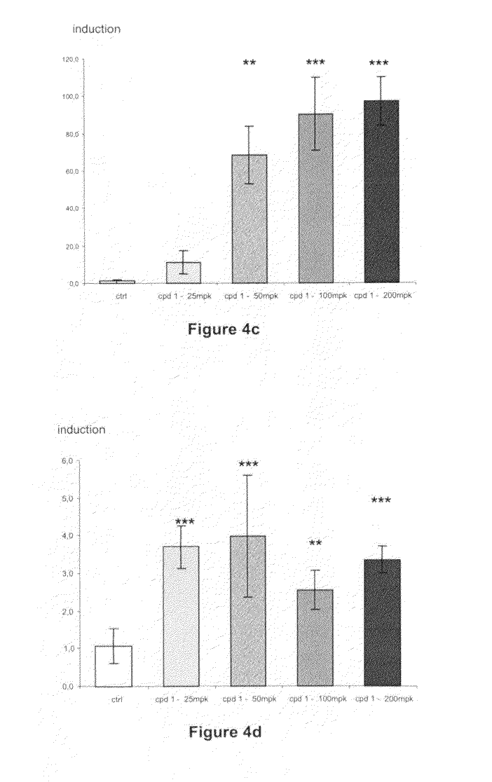

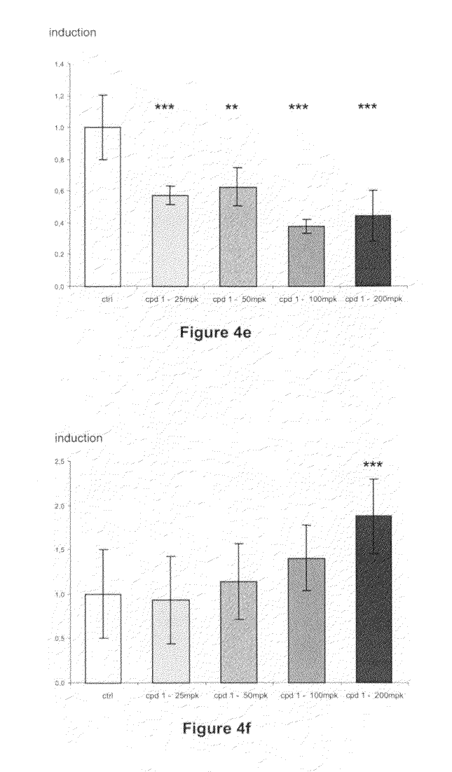

FIGS. 4a to 4f: In Vitro Evaluation of the Hypolipemic Properties of the Compounds According to the Invention

The effect of the compounds according to the invention is in vivo evaluated in humanized mouse with E2 isoform of apolipoprotein E (E2/E2 mouse).

The total plasma cholesterol and triglycerides levels were measured in dislipidemic E2/E2 mouse after a seven-day per os treatment with compounds according to the invention. These parameters were compared to the ones obtained with the control animals (animals not treated with the compounds according to the invention): the measured difference shows the effect of the compounds according to the invention on body weight, their hypolipemic effect as well.

FIG. 4a: Plasma cholesterol level after 7 days of treatment with compound 1, administered at 25, 50, 100 and 200 mpk

FIG. 4b: Plasma triglycerides level after 8 days of treatment with compound 1, administered at 25, 50, 100 and 200 mpk

The efficiency of the compounds according to the invention was also evaluated by measuring the expression of genes involved in lipid and/or glucid metabolism, in the hepatic and epididymal tissues. The expression levels relative to each gene were normalized regarding the expression level of reference genes (36B4 for hepatic tissue, and 18S for epididymal tissue). The induction factor, i.e. the ratio between the relative signal (induced by the compound according to the invention) and the average of the relative values obtained with the control group, is then calculated. The higher the induction factor is, the more the compound promotes hepatic gene expression. The final result is represented as the average of the induction values obtained with each experimental group.

FIG. 4c: Expression of PDK4 (isoform 4 of Pyruvate Dehydrogenase Kinase) in the hepatic tissue of the E2/E2 mouse, after 7 days of treatment with compound 1, administered at 4 doses (25, 50, 100, and 200 mpk)

FIG. 4d: Expression of ACO (acyl-CoA oxidase) in the hepatic tissue of the E2/E2 mouse, after 7 days of treatment with compound 1, administered at 4 doses (25, 50, 100, and 200 mpk)

FIG. 4e: Expression of ApoCIII (Apolipoprotein C3) in the hepatic tissue of a E2/E2 mouse, after 7 days of treatment with compound 1, administered at 4 doses (25, 50, 100, and 200 mpk)

FIG. 4f: Expression of PEPCK (PhosphoEnolPyruvate CarboxylKinase) in the hepatic tissue of the E2/E2 mouse, after 7 days of treatment with compound 1, administered at 4 doses (25, 50, 100, and 200 mpk)

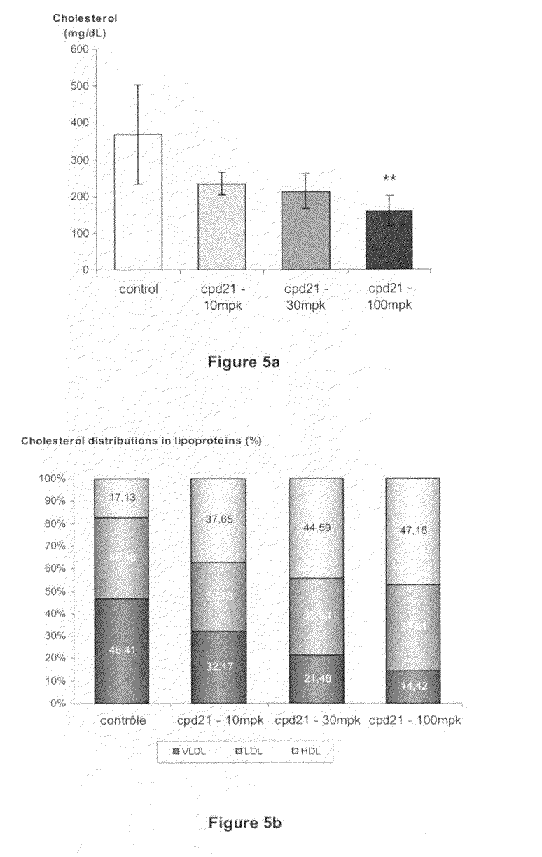

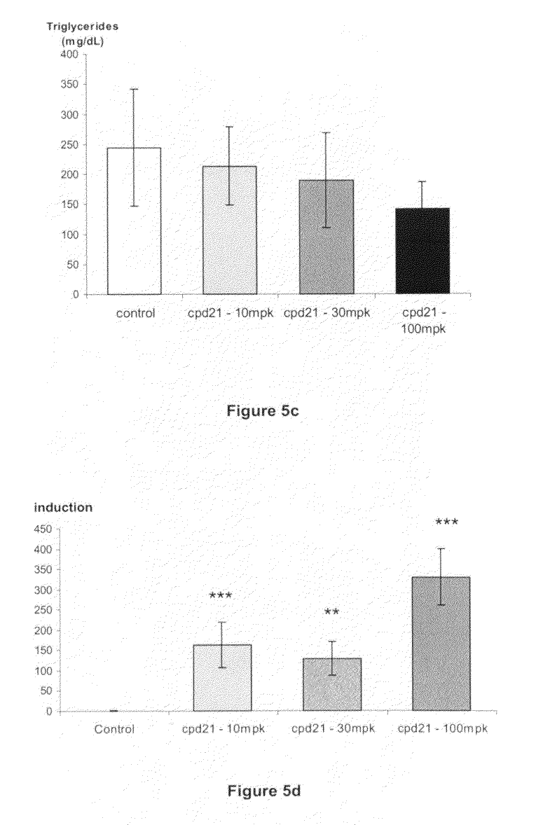

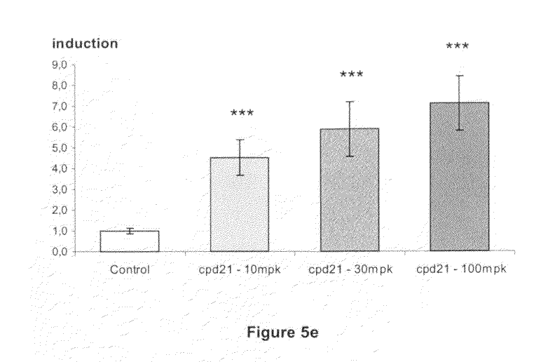

FIGS. 5a to 5e: In Vivo Evaluation of the Hypolipemic Properties of the Compounds According to the Invention, in the ApoE2/E2 Mouse

The effect of the compounds according to the invention is in vivo evaluated in humanized mouse with E2 isoform of apolipoprotein E (E2/E2 mouse).

The total plasma cholesterol and triglycerides levels were measured in dislipidemic E2/E2 mouse after a seven-day per os treatment with compounds according to the invention. These parameters were compared to the ones obtained with the control animals (animals not treated with the compounds according to the invention): the measured difference shows the effect of the compounds according to the invention on body weight, their hypolipemic effect as well.

FIG. 5a: Plasma cholesterol level after 7 days of treatment with compound 21, administered at 10, 30 and 100 mpk

FIG. 5b: Distribution of cholesterol in different plasma lipoprotein fractions after 7 days of treatment with compound 21, administered at 10, 30 and 100 mpk

FIG. 5c: Plasma triglycerides level after 7 days of treatment with compound 21, administered at 10, 30 and 100 mpk

The efficiency of the compounds according to the invention was also evaluated by measuring, in hepatic tissue, the expression of genes involved in lipid and/or glucid metabolism, in energy dissipation and in the anti-inflammatory response. The expression levels relative to each gene were normalized regarding the expression level of reference 36B4 gene. The induction factor, i.e. the ratio between the relative signal (induced by the compound according to the invention) and the average of the relative values obtained with the control group, is then calculated. The higher this induction factor is, the more the compound promotes gene expression. The final result is represented as the average of the induction values obtained with each experimental group.

FIG. 5d: Expression of PDK4 (isoform 4 of Pyruvate Dehydrogenase Kinase) in the hepatic tissue of the E2/E2 mouse, after 7 days of treatment with compound 21, administered at 10, 30 and 100 mpk

FIG. 5e: Expression of ACO (acyl-CoA oxidase) in the hepatic tissue of the E2/E2 mouse, after 7 days of treatment with compound 21, administered at 10, 30 and 100 mpk

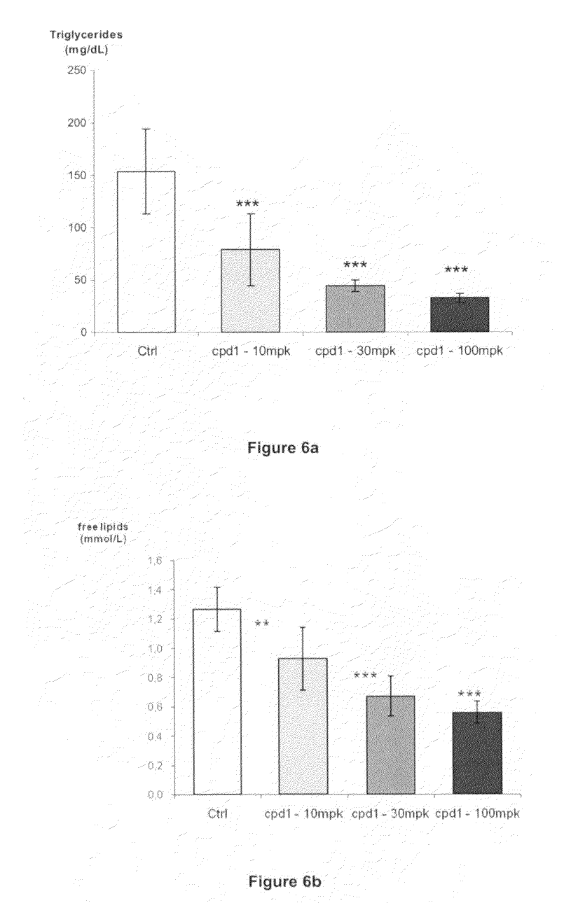

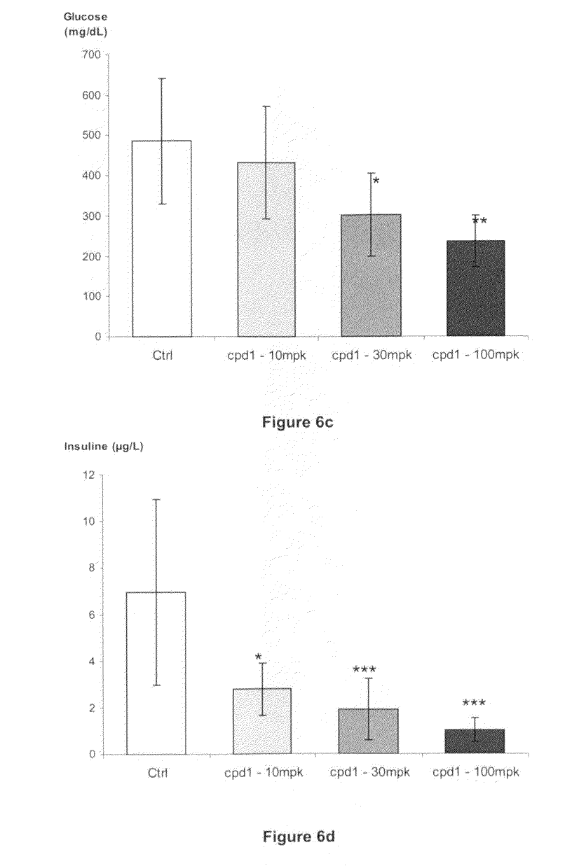

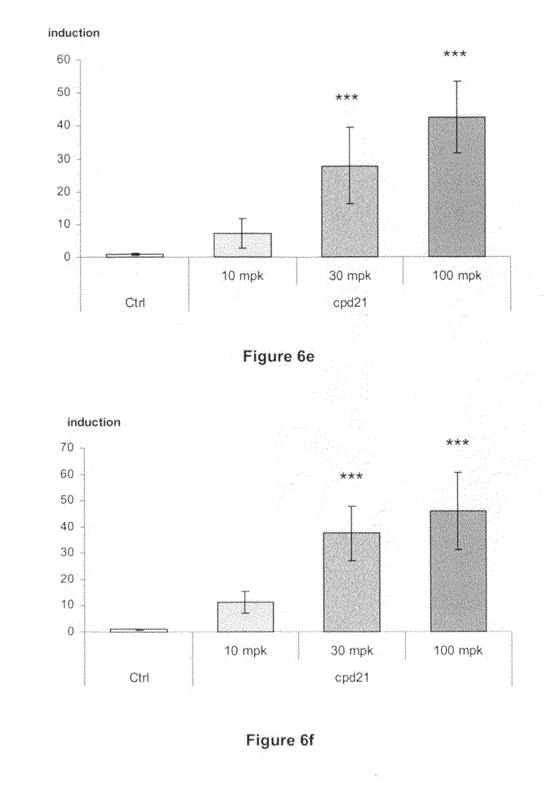

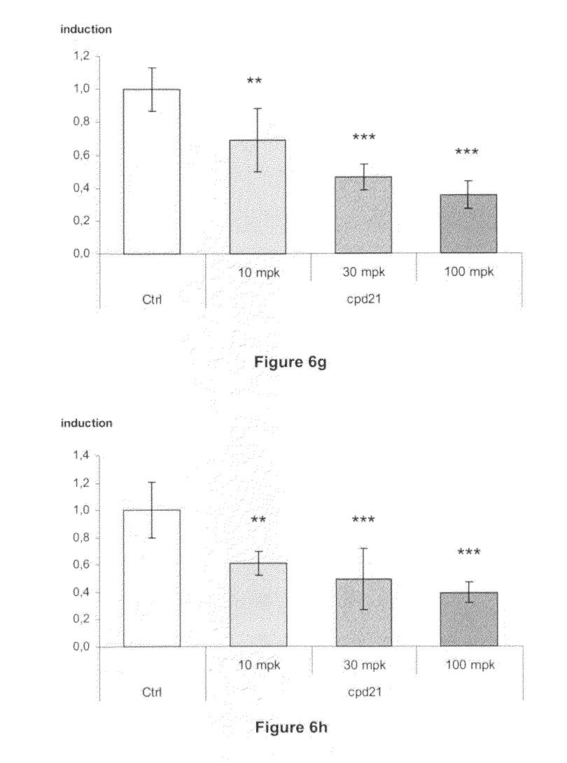

FIGS. 6a to 6h: In Vivo Evaluation, on the db/db Mouse, of Antidiabetic and Hypolipemic Properties of the Compounds According to the Invention.

The effects of the compounds according to the invention is in vivo evaluated by the measurement of the total cholesterol, triglycerides, and of the levels of plasma glucose and insulin after 28 days of a per os treatment with the compounds according to the invention. These parameters were compared to the ones obtained with the control animals (animals not treated with the compounds according to the invention): the measured difference shows the hypolipemic effect of the compounds according to the invention, and their effect on insulin-resistance as well.

FIG. 6a: Plasma triglycerides level after 28 days of a treatment with compound 1, administered at 10, 30 and 100 mpk

FIG. 6b: Plasma lipids level after 28 days of a treatment with compound 1, administered at 10, 30 and 100 mpk

FIG. 6c: Glycemia after 28 days of a treatment with the compound 1, administered at 10, 30 and 100 mpk

FIG. 6d: Insulinemia after 28 days of a treatment with the compound 1, administered at 10, 30 and 100 mpk

The efficiency of the compounds according to the invention was also evaluated by measuring, in hepatic tissue, the expression of genes involved in lipid and/or glucid metabolism, in energy dissipation and in the anti-inflammatory response. The expression levels relative to each gene were normalized regarding the expression level of reference 36B4 gene. The induction factor, i.e. the ratio between the relative signal (induced by the compound according to the invention) and the average of the relative values obtained with the control group, is then calculated. The higher this induction factor is, the more the compound promotes gene expression. The final result is represented as the average of the induction values obtained with each experimental group.

FIG. 6e: Expression of PDK4 (isoform 4 of Pyruvate Dehydrogenase Kinase) in the hepatic tissue of the db/db mouse, after 28 days of a treatment with compound 1, administered at 10, 30 and 100 mpk

FIG. 6f: Expression of CPT1b (Carnitine PalmitoylTransferase 1b) in the hepatic tissue of the db/db mouse, after 28 days of a treatment with compound 1, administered at 10, 30 and 100 mpk

FIG. 6g: Expression of ApoCIII (Apolipoprotein C3) in the hepatic tissue of the db/db mouse, after 28 days of a treatment with compound 1, administered at 10, 30 and 100 mpk

FIG. 6h: Expression of FGb (fibrinogen beta chain) in the hepatic tissue of the db/db mouse, after 28 days of a treatment with compound 1, administered at 10, 30 and 100 mpk

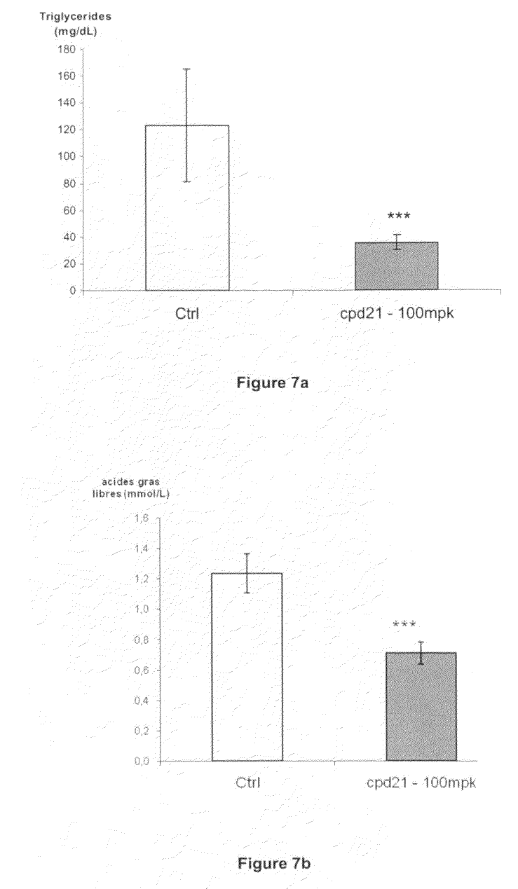

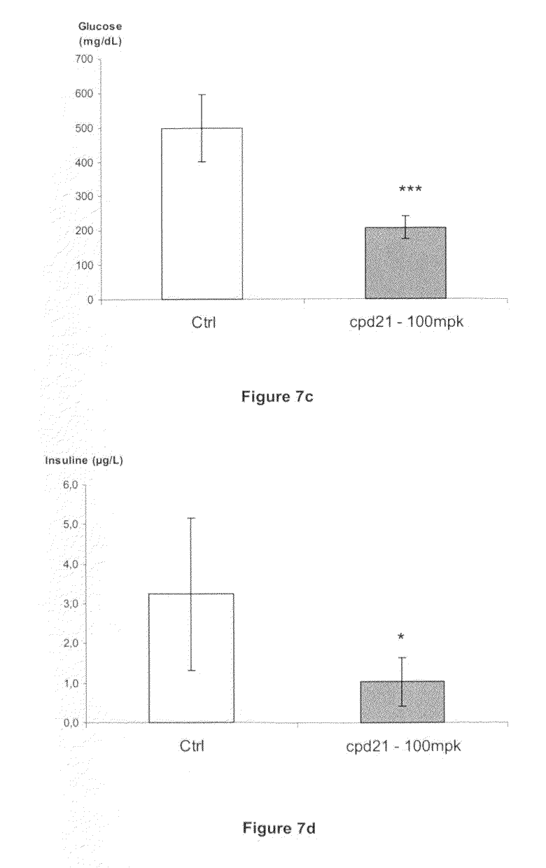

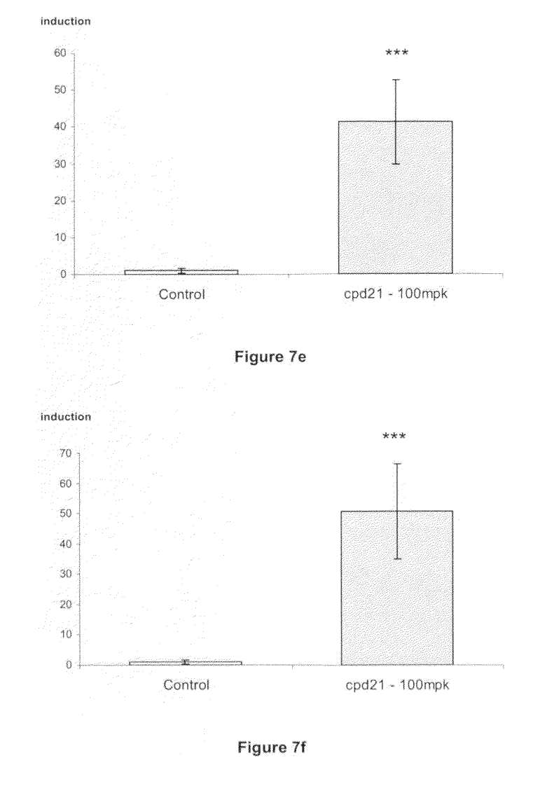

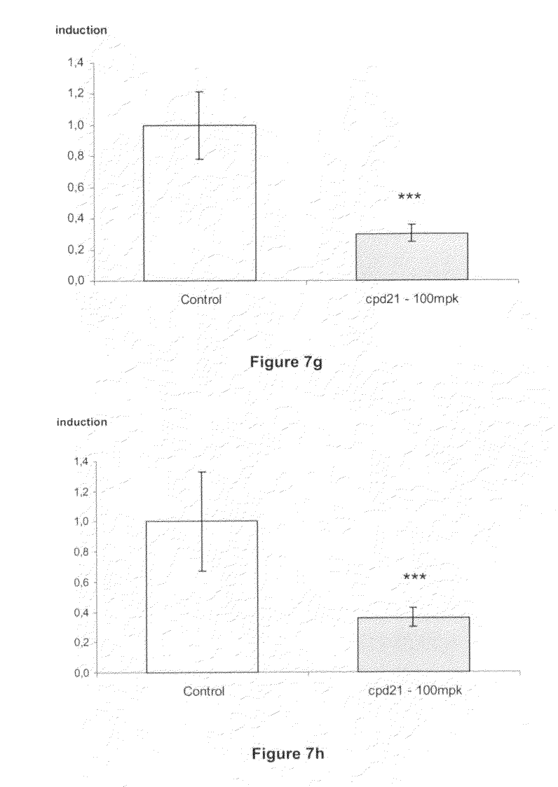

FIGS. 7a to 7i: In Vitro Evaluation of the Hypolipemic Properties of the Compounds According to the Invention

The effect of the compounds according to the invention is in vivo evaluated in the db/db mouse by measuring the plasma cholesterol, triglycerides, the level of plasma glucose and insulin after 28 days of a per os treatment with the compounds according to the invention. These parameters are compared to the ones obtained with the control animals (animals not treated with the compounds according to the invention): the measured difference shows the hypolipemic effect of the compounds according to the invention, and their effect on insulin-resistance.

FIG. 7a: Plasma triglycerides level after 28 days of a treatment with compound 21, administered at 100 mpk

FIG. 7b: Plasma lipids level after 28 days of a treatment with compound 21, administered at 100 mpk

FIG. 7c; Glycemia after 28 days of a treatment with compound 21, administered at 100 mpk

FIG. 7d: Insulinemia after 28 days of a treatment with compound 21, administered at 100 mpk

The efficiency of the compounds according to the invention was also evaluated by measuring, in the hepatic and adipose epididymal tissues, the expression of genes involved in glucid and/or lipid metabolism, in energy dissipation and in the anti-inflammatory response. The expression levels relative to each gene were normalized regarding the expression level of reference genes (36B4 for hepatic tissue, and 18S for epididymal tissue). The induction factor, i.e. the ratio between the relative signal (induced by the compound according to the invention) and the average of the relative values obtained with the control group, is then calculated. The higher the induction factor is, the more the compound promotes hepatic gene expression. The final result is represented as the average of the induction values obtained with each experimental group.

FIG. 7e: Expression of PDK4 (isoform 4 of Pyruvate Dehydrogenase Kinase) in the hepatic tissue of the db/db mouse, after 28 days of a treatment with compound 21, administered at 100 mpk

FIG. 7f: Expression of CPT1b (Carnitine PalmitoylTransferase 1b) in the hepatic tissue of the db/db mouse, after 28 days of a treatment with compound 21, administered at 100 mpk

FIG. 7g: Expression of ApoCIII (Apolipoprotein C3) in the hepatic tissue of the db/db mouse, after 28 days of a treatment with compound 21, administered at 100 mpk

FIG. 7h: Expression of FGb (fibrinogen beta chain) in the hepatic tissue of the db/db mouse, after 28 days of a treatment with compound 21, administered at 100 mpk

FIG. 7i: Expression of PEPCK (PhosphoEnolPyruvate CarboxyKinase) in the adipose epididymal tissue of the db/db mouse, after 28 days of a treatment with compound 21, administered at 100 mpk

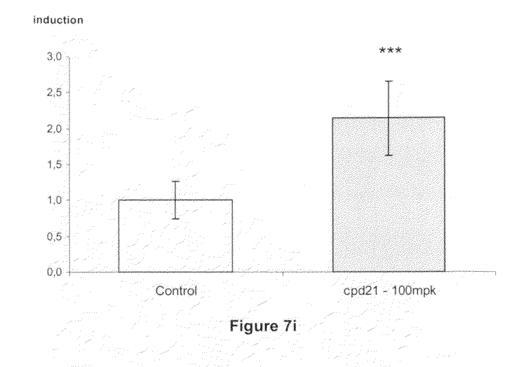

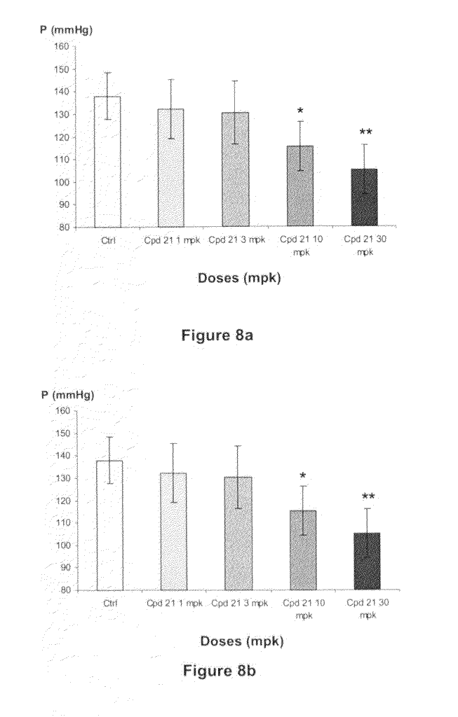

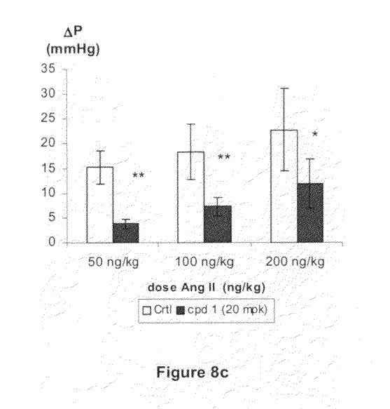

FIGS. 8a to 8b: In Vivo Evaluation of the Angiotensin II Antagonist Properties of the Compounds According to the Invention on Rats

FIG. 8a: Measurement of arterial pressure (P) of Wistar rats under perfusion of angiotensin II and intravenously treated with compound 1 (1, 3, 10, and 30 mpk).

The results, expressed in mm of Hg, express the arterial pressure measured after administration of the compounds according to the invention at the specified dose.

FIG. 8b: Measurement of arterial pressure (P) of Wistar rats under perfusion of angiotensin II and intravenously treated with compound 21 (1, 3, 10, and 30 mpk).

The results, expressed in mm of Hg, express the arterial pressure measured after administration of the compounds according to the invention at the specified dose.

FIG. 8c: Measurement of the difference of arterial pressure (ΔP) of Wistar rats under repeated administrations of angiotensin II (at 50, 100, and 200 ng/kg) and treated intravenously with compound 1 (20 mpk).

The results, expressed in mm of Hg, express the measured difference of arterial pressure between basal pressure and the pressure measured after the intravenous administration of angiotensin II (temporary hypertension) and after intravenous administration of compounds according to the invention at 20 mpk.

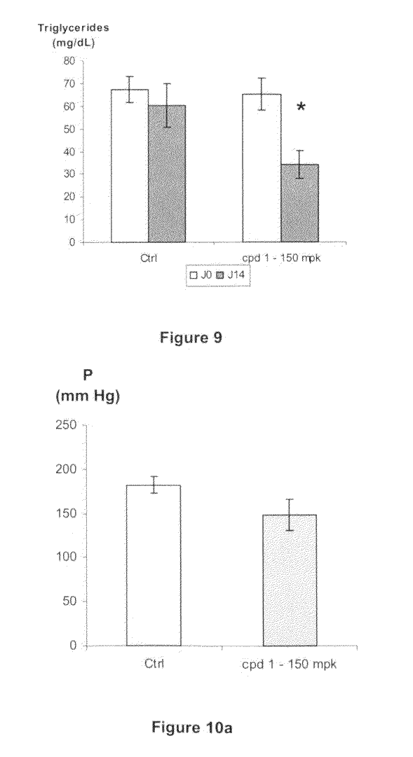

FIGS. 9, 10, and 11: In Vitro Evaluation of the Cardioprotective Properties of the Compounds According to the Invention

FIG. 9: Plasma triglyceride level after 14 days of treatment with compound 1, administrated at 150 mpk

The measured levels were compared to the ones obtained with the control animals (animals not treated with the compounds according to the invention): the measured difference shows the hypolipemic effect of the compounds according to the invention.

FIG. 10a: Measurement of arterial pressure (P) on SHR rats treated for 14 days with compound 1 (150 mpk), before repeated administrations of angiotensin II (50 ng/kg)

The results, expressed in mm of Hg, express the arterial pressure measured after 14 days of treatment.

FIG. 10b: Measurement of the difference of arterial pressure (ΔP) in SHR rats treated for 14 days with compound 1 (150 mpk), after three successive intravenous administrations of angiotensin II (50 ng/kg)

The results, expressed in mm of Hg, express the measured difference of arterial pressure between basal pressure and pressure measured after administration of angiotensin II (transient hypertension).

FIG. 10c: Measurement of the difference of arterial pressure (ΔP) in SHR rats treated for 14 days with compound 1 (150 mpk), after three successive intravenous administrations of angiotensin II (50 ng/kg)

The results, expressed in mm of Hg, express the measured difference of arterial pressure between basal pressure and pressure measured after administration of angiotensin II (transient hypertension).

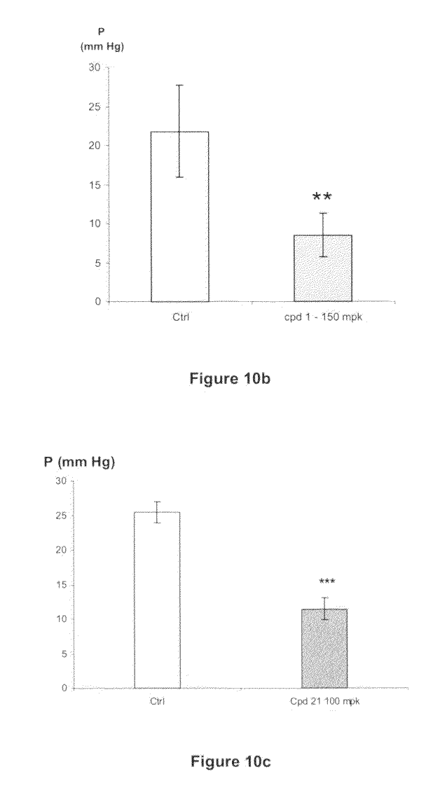

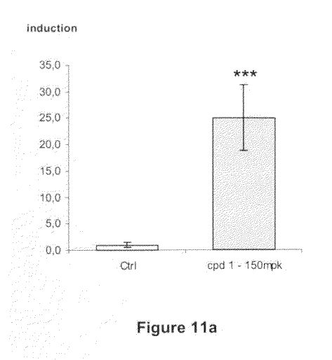

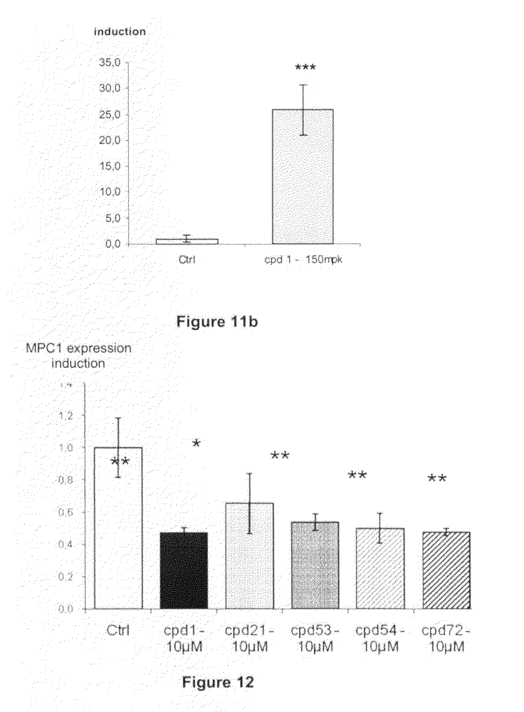

FIG. 11a: Expression of ACO in hepatic tissue, in SHR rats, after 14 days of treatment with compound 1, administered at 150 mpk

FIG. 11b: Expression of PDK4 in hepatic tissue, in SHR rats, after 14 days of treatment with compound 1, administered at 150 mpk

The expression levels of each gene are determined, and then normalized regarding the expression level of the reference 36B4 gene.

The induction factor, i.e. the ratio between the relative signal (induced by the compound according to the invention) and the average of the relative values obtained with the control group, was then calculated. The higher the induction factor is, the more the compound promotes gene expression. The final result was represented as the average of the induction values of each experimental group.

FIG. 12: In Vitro Evaluation of the Anti-Inflammatory Properties of the Compounds According to the Invention, by the Measurement of the MCP1 Secretion by Monocytes Treated with the Compounds According to the Invention and Stimulated by PMA

The anti-inflammatory effects relative to compounds according to the invention were evaluated by the measurement of MCP1 secretion (Monocyte chemotactic protein-1) by THP1 monocytes treated for 24 hours with the compounds according to the invention and stimulated simultaneously with PMA (Phorbal 12-myristate 13-acetate, induces an inflammatory reaction of the cells and their differentiation into macrophages). The more the expression of MCP-1 decreases, the more the compound according to the invention inhibits the inflammatory reaction.

FIG. 12: MCP1 (Monocyte chemotactic protein-1) secretion in THP1 monocytes, after 24 hours of a treatment with the compounds according to the invention at 10 μM

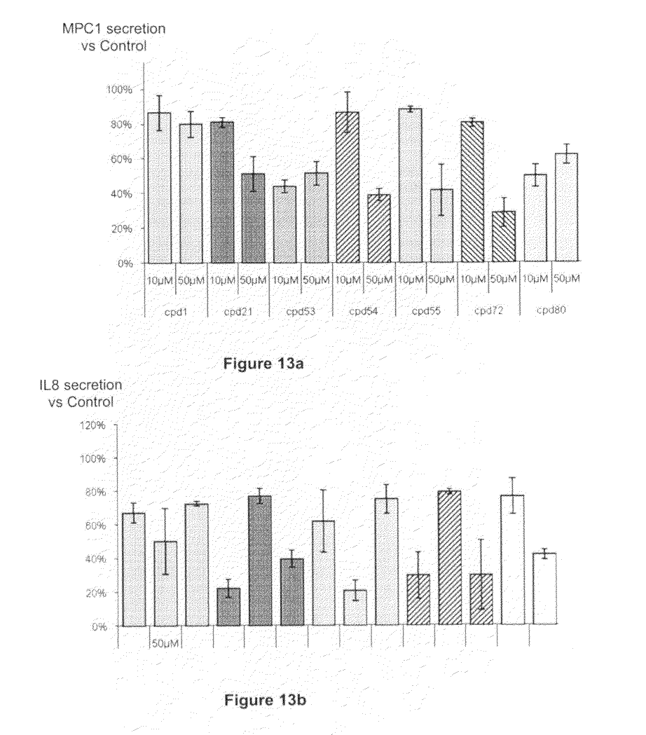

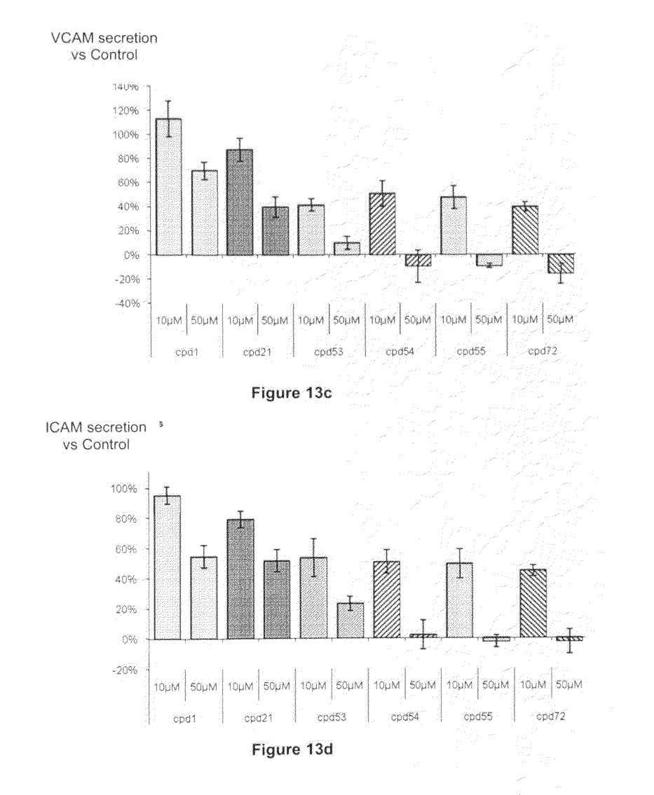

FIG. 13: In Vitro Evaluation of the Anti-Inflammatory Properties of the Compounds According to the Invention, by the Measurement of the Secretion of MCP1, IL8, VCAM and ICAM by HUVEC (Human Umbilical Vein Endothelial Cells) Treated by the Compounds According to the Invention and Stimulated with LPS

The anti-inflammatory effects relative to compounds according to the invention were evaluated by the measurement of the secretion of MCP1 (Monocyte chemotactic protein-1), d′IL8 (Interleukin 8), VCAM (Vascular Cell Adhesion Molecule) by HUVEC (Human Umbilical Vein Endothelial Cells) treated for 24 hours with LPS 1 μg/μl (Lipopolysaccharide, induces an inflammatory of cells). The more the inflammatory markers secretion decreases, the more the compound according to the invention inhibits the inflammatory reaction.

FIG. 13a: MCP1 secretion in HUVEC, after 24 hours of a treatment with the compounds according to the invention at 10 μM

FIG. 13b: IL8 secretion in HUVEC, after 24 hours of a treatment with the compounds according to the invention at 10 and 50 μM

FIG. 13c: VCAM secretion in HUVEC, after 24 hours of a treatment with the compounds according to the invention at 10 and 50 μM

FIG. 13d: ICAM secretion in HUVEC, after 24 hours of a treatment with the compounds according to the invention at 10 and 50 μM

### STATISTIC ANALYSES

The statistic studies of the different pharmacological tests were carried out using a univariate analysis of variance (ANOVA). The results are express in terms of a control group according to the value of parameter p: p<0.05 (noted *); p<0.01 (noted **); p<0.001 (noted ***).

### Examples

The following examples illustrate the invention without limiting it.

In these examples, different analyses of the identified compounds were carried out.

The melting points (MP) are given in Celsius degrees and, unless otherwise indicated, they were measured without recrystallization of the compound.

The purity of the products was controlled by thin-layer chromatography (TLC) and/or by HPLC (high-performance liquid chromatography).

The infra-red spectra (IR) were performed on inert support (germanium crystal).

The mass spectra were performed by ESI-MS (Electrospray Ionization—mass spectroscopy) or MALDI-TOF (Matrix Assisted Laser Desorption/lonization—Time of Flight).

The NMR spectra were recorded at 200 or 300 MHz in a deuterated solvent which was adjusted for each analysis: DMSO-d₆, CDCl₃ or Methanol-d4. The following abbreviations were used for interpreting the spectra: s for singlet, d for doublet, dd for dedoubled doublet, ddd for dedoubled dedoubled doublet, t for triplet, td for dedoubled triplet, q for quadruplet, quint for quintuplet, sext for sextuplet, m for multiplet or massive.

### Example 1

### General Procedure for the Preparation of Imidates

Method 1A: The appropriate nitrile (1eq) was added at 0° C. to a solution of anhydrous ethanol saturated with gaseous hydrochloric acid. The reaction mixture was stirred at 0° C. for 96 hours. The mixture was then dissolved in anhydrous diethyl ether and cooled at a temperature of −80° C. The precipitate of ethyl imidate hydrochloride was filtered and washed with diethyl ether at 20° C. The crystals were dried in a desiccator in the presence of P₂O₅.

Method 1B: The appropriate nitrile (1eq) was added at 0° C. to a solution of anhydrous ethanol saturated with gaseous hydrochloric acid (6.3eq). The reaction mixture was stirred for 18 hours at room temperature. The reaction mixture was evaporated under reduced pressure and dried in a desiccator.

### Example 1.1

### ethyl pentanimidate hydrochloride

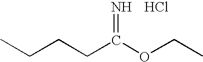

Prepared as a white powder following the general procedure previously described (method 1A) using valeronitrile.

Yield: 76%

IR: νC═N: 1650 cm⁻¹

NMR ¹H (DMSO-d₆): 0.87 (t, 3H, J=7.3 Hz); 1.23-1.35 (m, 5H); 1.56 (quint, 2H, J=7.3 Hz); 2.62 (t, 2H, J=7.3 Hz); 4.42 (q, 2H, J=7 Hz); 1117 (sl, 1H); 12.11 (sl, 1H).

### Example 1.2

### ethyl acetimidate hydrochloride

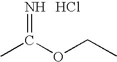

Prepared as a white powder following the general procedure previously described (method 1A) using acetonitrile.

Yield: 27%

MP: 112-114° C.

NMR ¹H (DMSO-d₆): 1.15 (t, 3H, J=7.3 Hz); 2.38 (s, 3H); 4.42 (q, 2H, J=7.3 Hz); 11.10 (sl, 1H); 12.12 (sl, 1H).

### Example 1.3

### ethyl propanimidate hydrochloride

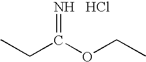

Prepared as a white powder following the general procedure previously described (method 1A) using propionitrile.

Yield: 29%

NMR ¹H (DMSO-d₆): 1.18 (t, 3H, J=7.3 Hz); 1.27 (t, 3H, J=7.6 Hz); 2.62 (q, 2H, J=7.3 Hz); 4.21 (q, 2H, J=7 Hz); 11.20 (sl, 1H); 12.02 (sl, 1H).

### Example 1.4

### ethyl butanimidate hydrochloride

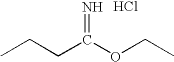

Prepared as a white powder following the general procedure previously described (method 1A) using butyronitrile.

Yield: 31%

MP: 74-79° C. NMR ¹H (DMSO-d₆): 0.87 (t, 3H, J=7.3 Hz); 1.27 (t, 3H, J=7.6 Hz); 1.62 (sext, 2H, J=7.3 Hz); 2.60 (t, 2H, J=7.3 Hz); 4.42 (q, 2H, J=7.6 Hz); 11.19 (sl, 1H); 12.11 (sl, 1H).

### Example 1.5

### ethyl 3-methylbutanimidate hydrochloride

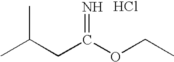

Prepared as a white powder following the general procedure previously described (method 1B) using 3-methylbutyronitrile.

Yield: 99%

NMR ¹H (DMSO-d₆): 0.92 (d, 6H, J=7.6 Hz); 1.18 (t, 3H, J=7.6 Hz); 2.04 (m, 1H 2.51 (d, 2H, J=5 Hz); 4.42 (q, 2H, J=7.6 Hz); 11.42 (sl, 2H).

### Example 1.6

### ethyl cyclopropylcarboximidate hydrochloride

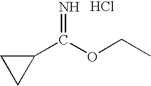

Prepared as a white powder following the general procedure previously described (method 1B) using cyclopropanecarbonitrile.

Yield: 83%

NMR ¹H (DMSO-d₆): 1.10-1.21 (m, 4H); 1.23 (t, 3H, J=7.6 Hz); 2.22 (m, 1H); 4.39 (q, 2H, J=7.6 Hz); 11.10 (sl , 1H); 12.18 (sl, 1H).

### Example 1.7

### ethyl 2-phenylacetimidate hydrochloride

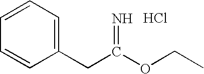

Prepared as a white powder following the general procedure previously described (method 1B) using 2-phenylacetonitrile.

Yield: 96%

NMR ¹H (DMSO-d₆): 1.30 (t, 3H, J=7.6 Hz); 4.02 (s, 2H); 4.40 (q, 2H, J=7.6 Hz 7.24-7.42 (m, 5H); 11.82 (sl, 2H).

### Example 1.8

### ethyl 2-(thiophen-3-yl)acetimidate hydrochloride

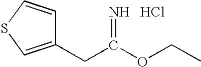

Prepared as a white powder following the general procedure previously described (method 1B) using 2-(thiophen-3-yl)acetonitrile.

Yield: 99%

NMR ¹H (DMSO-d₆): 1.26 (t, 3H, J=7.6 Hz); 4.02 (s, 2H); 4.42 (q, 2H, J=7.6 Hz 7.12 (dd, 1H, J=4 Hz, J=1 Hz); 7.20-7.50 (m, 2H); 7.52 (d, 1H, J=1 Hz); 7.59 (q, 1H, J=3 Hz, J=1 Hz).

### Example 2

### General Procedure for the Preparation of Amino Acid Esters

Aminocarboxylic acid (1eq) was added at 0° C. to the appropriate alcohol (methanol or ethanol) and the mixture was saturated with anhydrous hydrochloric acid. Thionyl chloride was then added drop by drop. The reaction mixture was stirred at reflux for 12 hours. The reaction mixture was concentrated under reduced pressure and diethyl ether was added to the crude residue. The resulting powder was filtered and washed with diethyl ether.

### Example 2.1

### 1-aminocyclopentanecarboxylic acid methyl ester hydrochloride

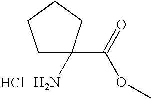

Prepared as a white powder following the general procedure previously described using cycloleucine and methanol.

Yield: 91%

Rf (dichloromethane/methanol 9/1): 0.5

MP: 157-159° C. IR: νCO: 1742 cm⁻¹

NMR ¹H (DMSO-d₆): 1.68-1.84 (m, 6H); 2.04 (m, 2H); 3.71 (s, 3H).

### Example 2.2

### 1-aminocyclopentanecarboxylic acid ethyl ester hydrochloride

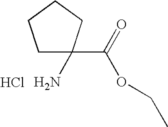

Prepared as a white powder following the general procedure previously described using cycloleucine and ethanol.

Yield: 82%

Rf (dichloromethane/methanol 9/1): 0.5

IR: νCO: 1736 cm⁻¹

NMR ¹H (DMSO-d₆): 1.22 (t, 3H, 7 Hz); 1.70-2.13 (m, 8H); 4.17 (q, 2H, 7 Hz); 8.83 (s, 3H).

### Example 2.3

### 1-aminocyclohexanecarboxylic acid methyl ester hydrochloride

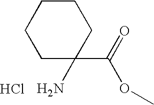

Prepared as a white powder following the general procedure previously described using 1-aminocyclohexanecarboxylic acid and methanol.

Yield: 69%

Rf (dichloromethane/methanol 9/1): 0.5

MP: 210° C.

IR: νCO: 1741 cm⁻¹

NMR ¹H (DMSO-d₆): 1.38-1.97 (m, 10H); 3.73 (s, 3H); 8.82 (sl, 3H).

### Example 2.4

### 1-aminoisobutyric acid methyl ester hydrochloride

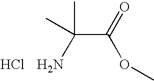

Prepared as a viscous oil following the general procedure previously described using 1-aminoisobutyric acid and methanol.

Yield: 81%

Rf (dichloromethane/methanol 9/1): 0.5

IR: νCO: 1746 cm⁻¹

NMR ¹H (CDCI₃): 1.70 (s, 6H); 3.80 (s, 3H).

### Example 2.5

### DL-2-phenylglycine methyl ester hydrochloride

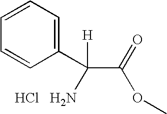

Prepared following the general procedure previously described using DL-2-phenylglycine and methanol.

Yield: 67%

Rf (dichloromethane/methanol 9/1): 0.5

MP: 207-209° C.

IR: νCO: 1742 cm⁻¹

NMR ¹H (DMSO-d₆): 3.69 (s, 3H); 5.23 (s, 1H); 7.43-7.55 (m, 5H); 9.17 (s, 3H).

### Example 2.6

### methyl 2-amino-2-ethylbutanoate hydrochloride

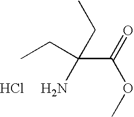

Prepared following the general procedure previously described using 2-amino-2-ethylbutanoic acid and methanol.

Yield: 47.5%

Rf (dichloromethane/methanol 9/1): 0.5

NMR ¹H (DMSO-d₆): 1.01 (t, 6H, J=7.6 Hz); 1.97 (q, 4H, J=7.6 Hz); 3.80 (s, 3H).

### Example 3

### General Procedure for the Preparation of Imidazolones

Imidate hydrochloride and aminoacid ester were separately neutralized by preliminary washing with a sodium carbonate solution and extracted with dichloromethane. The organic layers were separately dried over magnesium sulfate and evaporated under reduced pressure. To a solution of the ester (1eq) in xylene and acetic acid (0.06eq) was added the imidate (1eq). The reaction mixture was stirred at reflux for 6 hours. The mixture was evaporated under reduced pressure and the residue was chromatographed over silica gel.

### Example 3.1

### 2-methyl-4-spirocyclopentyl-1H-imidazol-5(4H)-one

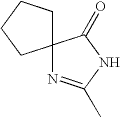

Prepared following the general procedure previously described using 1-aminocyclopentanecarboxylic acid methyl ester hydrochloride (example 2.1) and ethyl acetimidate hydrochloride (example 1.2). The product was chromatographed over silica gel (eluent dichloromethane/methanol 98/2, then 97/3, then 96/4). The product was obtained as a yellow oil.

Yield: 20%

NMR ¹H (CDCl₃): 1.60-2.15 (m, 8H); 2.16 (s, 3H).

### Example 3.2

### 2-ethyl-4-spirocyclopentyl-1H-imidazol-5(4H)-one

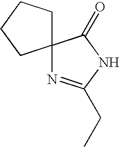

Prepared following the general procedure previously described using 1-aminocyclopentanecarboxylic acid methyl ester hydrochloride (example 2.1) and ethyl propanimidate hydrochloride (example 1.3). The product was chromatographed over silica gel (eluent dichloromethane/methanol 98/2, then 95/5, then 90/10) The product was obtained as anoil.

Yield: 20%

NMR ¹H (CDCl₃): 1.34 (t, 3H, J=7 Hz); 1.75-2.04 (m, 8H); 2.50 (q, 2H, J=7 HZ).

### Example 3.3

### 2-propyl-4-spirocyclopentyl-1H-imidazol-5(4H)-one

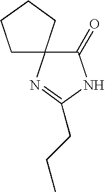

Prepared following the general procedure previously described using 1-aminocyclopentanecarboxylic acid methyl ester hydrochloride (example 2.1) and ethyl butanimidate hydrochloride (example 1.4). The product was chromatographed over silica gel (eluent dichloromethane/methanol 98/2, then 97/3, then 96/4). The product was obtained as an oil.

Yield: 32%

NMR ¹H (CDCl₃): 1.05 (t, 3H, J=7 Hz); 1.75 (sext, 2H, J=7 Hz); 1.80-2.02 (m, 8H); 2.49 (t, 2H, J=7 Hz).

### Example 3.4

### 2-butyl-4-spirocyclopentyl-1H-imidazol-5(4H)-one

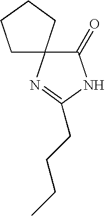

Prepared following the general procedure previously described using 1-aminocyclopentanecarboxylic acid methyl ester hydrochloride (example 2.1) and ethyl pentanimidate hydrochloride (example 1.1). The product was chromatographed over silica gel (eluent dichloromethane/methanol 95/5). The product was obtained as an oil.

Yield: 88%

Rf (dichloromethane/methanol 95/5): 0.25

IR: νCO: 1725 cm⁻¹

NMR ¹H (CDCl₃): 0.95 (t, 3H, J=7.3 Hz); 1.4 (sext, 2H, J=7.6 Hz); 1.66 (quint, 2H, J=7.9 Hz); 1.80-1.94 (m, 8H); 2.46 (t, 2H, J=7.6 Hz); 9.38 (s, 1H).

### Example 3.5

### 2-butyl-4-spirocyclohexyl-1H-imidazol-5(4H)-one

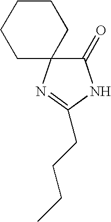

Prepared following the general procedure previously described using methyl 1-aminocyclohexanecarboxylic acid methyl ester hydrochloride (example 2.3) and ethyl pentanimidate hydrochloride (example 1.1). The product was chromatographed over silica gel (eluent dichloromethane/methanol 95/5) and obtained as a white powder.

Yield: 49%

Rf (dichloromethane/methanol 95/5): 0.25

MP: 123-125° C. IR: νCO: 1732 cm⁻¹

NMR ¹H (CDCl₃): 0.95 (t, 3H, J=7.3 Hz); 1.39 (sext, 2H, J=7.6 Hz); 1.40-1.73 (m, 12H); 2.48 (t, 2H, J=7.6 Hz); 9.32 (s, 1H).

### Example 3.6

### 2-butyl-4,4-dimethyl-1H-imidazol-5(4H)-one

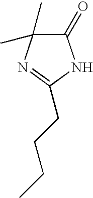

Prepared following the general procedure previously described using 1-aminoisobutyric acid methyl ester hydrochloride (example 2.4) and ethyl pentanimidate hydrochloride (example 1.1). The product was chromatographed over silica gel (eluent dichloromethane/methanol 95/5).The product was obtained as an oil.

Yield: 28%

Rf (dichloromethane/methanol 95/5): 0.25

IR: νCO: 1733 cm⁻¹

NMR ¹H (CDCl₃): 0.94 (t, 3H, J=7.3 Hz); 1.32 (s, 6H); 1.39 (quint, 2H, J=7.6 Hz); 1.65 (sext, 2H, J=7.6 Hz); 2.46 (t, 2H, J=7.6 Hz); 9.64 (s, 1H).

### Example 3.7

### 2-butyl-4-phenyl-1H-imidazol-5(4H)-one

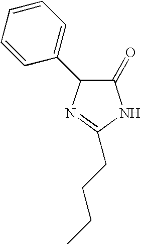

Prepared following the general procedure previously described using DL-2-phenylglycine methyl ester hydrochloride (example 2.5) and ethyl pentanimidate hydrochloride (example 1.1). The product was chromatographed over silica gel (eluent dichloromethane/methanol 95/5). The product was obtained as a white powder.

Yield: 5%

Rf (dichloromethane/methanol 95/5): 0.25

MP: 211-220° C. IR: νCO: 1732 cm⁻¹

NMR ¹H (DMSO-d₆): 0.92 (t, 3H, J=7.3 Hz); 1.33 (sext, 2H, J=7.6 Hz); 1.65 (quint, 2H, J=7.9 Hz); 2.57 (t, 2H, J=7.3 Hz); 5.13 (s, 1H); 7.20-7.70 (m, 5H); 8.66 (s, 1H).

### Example 3.8

### 2-isobutyl-4-spirocyclopentyl-1H-imidazol-5(4H)-one

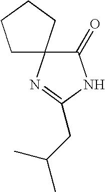

Prepared following the general procedure previously described using 1-aminocyclopentanecarboxylic acid methyl ester hydrochloride (example 2.1) and ethyl 3-methylbutanimidate hydrochloride (example 1.5). The product was chromatographed over silica gel (eluent dichloromethane/methanol 98/2, then 97/3, then 96/4). The product was obtained as a yellow oil.

Yield: 39%

NMR ¹H (CDCl₃): 1.32 (d, 6H, J=7.6 Hz); 1.61 (m, 2H); 1.71-1.96 (m, 8H); 2.10 (m, 1H).

### Example 3.9

### 2-benzyl-4-spirocyclopentyl-1H-imidazol-5(4H)-one

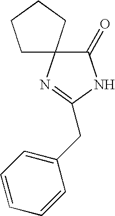

Prepared following the general procedure previously described using 1-aminocyclopentanecarboxylic acid methyl ester hydrochloride (example 2.1) and ethyl 2-phenylacetimidate hydrochloride (example 1.7). The product was chromatographed over silica gel (eluent dichloromethane/methanol 99/1, then 97/3). The product was obtained as a yellow oil.

Yield: 28%

NMR ¹H (CDCl₃): 1.74-2.07 (m, 8H); 3.75 (s, 2H); 7.22-7.41 (m, 5H).

### Example 3.10

### 2-cyclopropyl-4-spirocyclopentyl-1H-imidazol-5(4H)-one

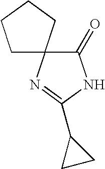

Prepared following the general procedure previously described using 1-aminocyclopentanecarboxylic acid methyl ester hydrochloride (example 2.1) and ethyl cyclopropylcarboximidate hydrochloride (example 1.6). The product was chromatographed over silica gel (eluent dichloromethane/methanol 95/5). The product was obtained as an oil.

Yield: 31%

NMR ¹H (DMSO-d₆): 0.95 (m, 4H); 1.50-1.84 (m, 8H); 2.12 (m, 1H).

### Example 3.11

### 2-(thiophen-3-yl)-4-spirocyclopentyl-1H-imidazol-5(4H)-one

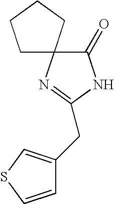

Prepared following the general procedure previously described using 1-aminocyclopentanecarboxylic acid methyl ester hydrochloride (example 2.1) and ethyl 2-(thiophen-3-yl)acetimidate hydrochloride (example 1.8). The product was chromatographed over silica gel (eluent dichloromethane/methanol 99/1, then 97/3). The product was obtained as a yellow oil.

Yield: 21%

NMR ¹H (CDCl₃): 1.78-2.05 (m, 8H); 3.85 (s, 2H); 7.02 (dd, 1H, J=4 Hz, J=1 Hz); 7.20 (d,1H, J=1 Hz); 7.38 (q, 1H, J=3 Hz, J=1 Hz).

### Example 3.12

### 2-butyl-4,4-diethyl-1H-imidazol-5(4H)-one

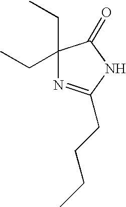

Prepared following the general procedure previously described using methyl 2-amino-2-ethylbutanoate hydrochloride (example 2.6) and ethyl pentanimidate hydrochloride (example 1.1). The product was chromatographed over silica gel (gradient of elution dichloromethane/methanol 100/0 to 98/2). The product was obtaines as an oil.

Yield: 43.3%

NMR ¹H (CDCl₃): 0.76 (t, 3H, J=7.6 Hz); 0.93 (m, 6H); 1.4 (m, 2H); 1.74 (m, 6H); 2.25 (t, 1H, J=7.3 Hz); 2.52 (t, 1H, J=7.7 Hz).

### Example 4

### General Procedure for the Preparation of Alkyl Ester Iodides

Alkyl ester iodides were prepared via reaction between methyl 2-methylpropanoate and appropriate alkyl diodide in the presence of butylithium and diisopropylamine according to the following process: under inert atmosphere, N,N-diisopropylamine (1.1eq) was dissolved in tetrahydrofuran (10eq). To the solution cooled down to 0° C. was added n-butyllithium (1.1eq) drop by drop. The solution was then cooled to −70° C. before adding 2-methylpropanoic acid (1eq). The mixture was stirred at −70° C. for 15 minutes. The appropriate diiodated derivative (2eq) was added drop by drop at −70° C., and then the reaction mixture was gradually warmed to room temperature and stirred for 20 hours. The solution was then hydrolysed by adding HCl 2N to reach acidic pH. The aqueous layer was extracted with ethyl acetate. The organic layer was washed with brine, dried over sodium sulfate, filtered, and evaporated under reduced pressure. The residue was chromatographed over silica gel.

### Example 4.1

### methyl 2,2-dimethyl-5-iodo-pentanoate

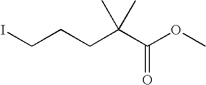

Prepared following the general procedure previously described using methyl 2-methylpropanoate and 1,3-diiodopropane. The residue was chromatographed over silica gel (eluent cyclohexane). The product was obtained as a pale yellow oil.

Yield: 79%

Rf (cyclohexane/ethyl acetate 98/2): 0.32

NMR ¹H (CDCl₃): 1.20 (s, 6H); 1.62 (m, 2H); 1.78 (m, 2H); 3.15 (t, 2H, J=7 Hz); 3.69 (s, 2H).

### Example 4.2

### methyl 2,2-dimethyl-8-iodo-octanoate

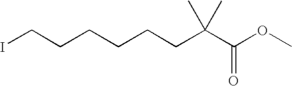

Prepared following the general procedure previously described using methyl 2-methylpropanoate and 1,6-diiodohexane. The residue was chromatographed over silica gel (eluent cyclohexane). The product was obtained as a colorless oil.

Yield: 82%

Rf (cyclohexane/ethyl acetate 98/2): 0.43

NMR ¹H (CDCl₃): 1.15 (s, 6H); 1.10-1.60 (m, 8H); 1.8 (m, 2H); 3.19 (m, 2H); 3.65 (s, 3H).

### Example 4.3

### methyl 2,2-dimethyl-3-iodo-propanoate

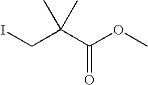

Prepared following the general procedure previously described using methyl 2-methylpropanoate and diiodomethane. The product was chromatographed over silica gel (eluent cyclohexane, and then cyclohexane/ethyl acetate 9/1). The product was obtained as an orange oil.

Yield: 51%

Rf (cyclohexane/ethyl acetate 98/2): 0.45

NMR ¹H (CDCl₃): 1.35 (s, 6H); 3.35 (s, 2H); 3.72 (s, 3H).

### Example 5

### General Procedure for the Preparation of Phenethyl Bromides

Phenethyl bromides were prepared in 2 steps using the appropriate 2-(hydroxyphenyl)ethanol: the phenol function was alkylated, and then the hydroxyl function of the alkyl chain was brominated.

Phenol Function Substitution

To a solution of the appropriate phenol (1eq) and the suitable brominated derivative (1eq) in acetonitrile was added a suspension of potassium carbonate. The reaction mixture was stirred at reflux for 12 hours. The mixture was cooled to room temperature, acidified with a hydrochloric acid 1N solution, and then extracted with ethyl acetate. The combined organic layers were dried over magnesium sulfate, and evaporated under reduced pressure. The residue was chromatographed over silica gel.

Bromination

The product previously prepared (1eq) and triphenylphosphine (1.2eq) were dissolved in dichloromethane. The reaction mixture was cooled to 0° C. before adding bromine (1.2eq). The reaction mixture was stirred at room teperature for 5 hours, then evaporated under reduced pressure. The residue was chromatographed over silica gel.

### Example 5.1

### ethyl 2-(2-(2-bromoethyl)phenoxy)-2-methylpropanoate

### 5.1.1 ethyl 2-(2-(2-hydroxyethyl)phenoxy)-2-methylpropanoate

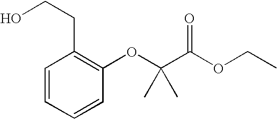

Prepared following the general substitution procedure previously described from 2-(2-hydroxyethyl)phenol and ethyl 2-bromoisobutyrate. The product was chromatographed over silica gel (eluent cyclohexane/ethyl acetate 70/30). The product was obtained as a colorless oil.

Yield: 68%

Rf (cyclohexane/ethyl acetate 7/3): 0.30

IR: νCO: 1733 cm⁻¹

NMR ¹H (CDCl₃): 1.23 (t, 3H, J=7 Hz); 1.65 (s, 6H); 2.93 (t, 2H, J=6.2 Hz); 3.85 (t, 2H, J=6.2 Hz); 4.21 (q, 2H, J=7 Hz); 6.68 (d, 1H, J=8.3 Hz); 6.93 (t, 1H, J=6.4 Hz); 7.05-7.19 (m, 2H).

### 5.1.2 ethyl 2-(2-(2-bromoethyl)phenoxy)-2-methylpropanoate

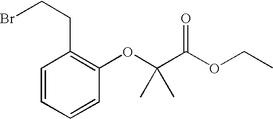

Prepared following the general bromination procedure previously described from ethyl 2-(2-(2-hydroxyethyl)phenoxy)-2-methylpropanoate (example 5.1.1). The product was chromatographed over silica gel (eluent cyclohexane/ethyl acetate 95/5). The product was obtained as a colorless oil.

Yield: 33%

Rf (cyclohexane/ethyl acetate 9/1): 0.40

IR: νCO: 1736 cm⁻¹

NMR ¹H (CDCl₃): 1.22 (t, 3H, J=7 Hz); 1.65 (s, 6H); 3.19 (t, 2H, J=7.9 Hz); 3.62 (t, 2H, J=7.9 Hz); 4.22 (q, 2H, J=7 Hz); 6.66 (d, 1H, J=8.2 Hz); 6.92 (t, 1H, J=7.3 Hz); 7.15 (m, 2H).

### Example 5.2

### ethyl 2-(3-(2-bromoethyl)phenoxy)-2-methylpropanoate

### 5.2.1 ethyl 2-(3-(2-hydroxyethyl)phenoxy)-2-methylpropanoate

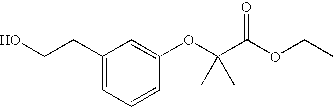

Prepared following the general substitution procedure previously described from 3-(2-hydroxyethyl)phenol and ethyl 2-bromoisobutyrate. The product was chromatographed over silica gel (eluent cyclohexane/ethyl acetate 70/30). The product was obtained as a colorless oil.

Yield: 71%

Rf (cyclohexane/ethyl acetate 6/4): 0.45

IR: νCO: 1732 cm⁻¹

NMR ¹H (CDCl₃): 1.22 (t, 3H, J=7 Hz); 1.57 (s, 6H); 2.75 (t, 2H, J=6.7 Hz); 3.81 (t, 2H, J=6.7 Hz); 4.24 (q, 2H, J=7 Hz); 6.65 (d, 1H, J=8.2 Hz); 6.71 (s, 1H); 6.81 (d, 1H, J=7.6 Hz); 7.13 (t,1H, J=7.9 Hz).

### 5.2.2 ethyl 2-(3-(2-bromoethyl)phenoxy)-2-methylpropanoate

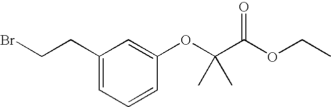

Prepared following the general bromination procedure previously described from ethyl 2-(3-(2-hydroxyethyl)phenoxy)-2-methylpropanoate (example 5.2.1). The product was chromatographed over silica gel (eluent cyclohexane/ethyl acetate 98/2, and then cyclohexane/ethyl acetate 95/5). The product was obtained as a colorless oil.

Yield: 29%

Rf (cyclohexane/ethyl acetate 98/2): 0.3

IR: νCO: 1734 cm⁻¹

NMR ¹H (CDCl₃): 1.26 (t, 3H, J=7 Hz); 1.63 (s, 6H); 3.10 (t, 2H, J=7.6 Hz); 3.53 (t, 2H, J=7.9 Hz); 4.24 (q, 2H, J=7.3 Hz); 6.72 (m, 2H); 6.83 (d, 1H, J=7.6 Hz); 7.18 (t, 1H, J=7.6 Hz).

### Example 5.3

### ethyl 2-(4-(2-bromoethyl)phenoxy)-2-methylpropanoate

### 5.3.1 ethyl 2-(4-(2-hyd roxyethyl)phenoxy)-2-methylpropanoate

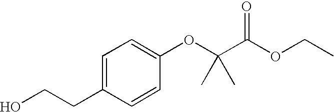

Prepared following the general substitution procedure previously described from 4-(2-hydroxyethyl)phenol and ethyl 2-bromoisobutyrate. The product was chromatographed over silica gel (eluent cyclohexane/ethyl acetate 70/30). The product was obtained as a colorless oil.

Yield: 97%

Rf (cyclohexane/ethyl acetate 7/3): 0.2

IR: νCO: 1732 cm⁻¹

NMR ¹H (CDCl₃): 1.26 (t, 3H, J=7.3 Hz); 1.59 (s, 6H); 2.8 (t, 2H, J=6.7 Hz); 3.81 (m, 2H); 4.24 (q, 2H, J=7 Hz); 6.80 (d, 2H, J=8.5 Hz); 7.1 (d, 2H, J=8.5 Hz).

### 5.3.2 ethyl 2-(4-(2-bromoethyl)phenoxy)-2-methylpropanoate

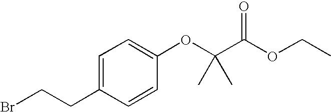

Prepared following the general bromination procedure previously described from ethyl 2-(4-(2-hydroxyethyl)phenoxy)-2-methylpropanoate (example 5.3.1). The product was chromatographed over silica gel (eluent cyclohexane/ethyl acetate 95/5). The product was obtained as a colorless oil.

Yield: 88%

Rf (cyclohexane/ethyl acetate 95/5): 0.30

IR: νCO: 1733 cm⁻¹

NMR ¹H (CDCl₃): 1.26 (t, 3H, J=7 Hz); 1.60 (s, 6H); 3.10 (t, 2H, J=7.6 Hz); 3.53 (t 2H, J=7.9 Hz); 4.24 (q, 2H, J=7.3 Hz); 6.80 (d, 2H, J=8.5 Hz); 7.08 (d, 2H, J=8.5 Hz).

### Example 6

### General Procedure for the Preparation of Biarylmethyl Bromides

Biarylmethyl bromides were prepared in several steps according to the following methods:

Method 6A: using the appropriate bromophenol with an alkylated phenol function. The O-alkylation was followed by a Suzuki reaction. The aromatic methyl was then free-radical brominated.

Bromophenol Substitution

To a solution of the appropriate bromophenol (1eq) and suitable halogenated derivative (1eq) in acetonitrile was added a suspension of potassium carbonate (3eq). The reaction mixture was stirred at reflux for 12 hours. The mixture was cooled to room temperature, acidified with a hydrochloric acid 1N solution, and then extracted with ethyl acetate. The combined organic layers were dried over magnesium sulfate, and evaporated under reduced pressure. The residue was chromatographed over silica gel.

Suzuki Reaction

The Tetrakis(triphenylphosphine)palladium (Pd[P(Ph)₃]₄) derivative (0.01eq) and the O-alkylated product previously prepared (1eq) were heated to 120° C. with tetrabutylammonium bromide (3.7eq) until a brown coloration was reached. A potassium carbonate solution (2N) (1eq) and the appropriate boronic acid (1.15eq) were then added. The reaction mixture was stirred at 120° C. for 30 minutes. The temperature was then reduced to 60° C. and diethyl ether was added with caution. The mixture was stirred vigorously for a few minutes and cooled to room temperature. The organic layer was separated. The aqueous layer was washed several times with ether. The combined organic layers were dried over magnesium sulfate, and evaporated under reduced pressure. The residue was chromatographed over silica gel.

Methyl Bromination

N-bromosuccinimide (1.2eq), benzoyl peroxide (0.08eq), and the biphenylmethyl derivative previously prepared (1eq) were dissolved in chloroform. The reaction mixture was stirred at reflux under a light source (500 W). The mixture turned brown after 15 minutes of stirring at reflux and the color gradually fades. The mixture is cooled to room temperature and washed with water. The aqueous layer was extracted with dichloromethane. The combined organic layers were dried over magnesium sulfate, and evaporated under reduced pressure. The residue was chromatographed over silica gel. Analyses of the purified product may show the presence of a part of the derivative that also carries a bromine atom on the aromatic cycle.

Method 6B: using the appropriate bromophenol. The Suzuki reaction was followed by 0-alkylation. The aromatic methyl was then free-radical brominated.

Suzuki Reaction

To a solution of the suitable boronic acid (1.25eq) and the appropriate bromophenol (1eq) in 1.2-dimethoxyethane (100eq) under nitrogen atmosphere was added the tetrakis(triphenylphosphine)palladium (Pd[P(Ph)₃]₄) derivative (0.034eq). The reaction mixture was stirred at reflux for 12 hours. Water was added and the mixture was extracted three times with ethyl acetate. The combined organic layers were dried over sodium sulfate, and evaporated under reduced pressure. The residue was chromatographed over silica gel.

Phenol Substitution

To a solution of the phenylphenol previously prepared (1eq) in dimethylformamide was added the appropriate brominated derivative (4eq) at 80° C. before adding potassium carbonate (3eq). The reaction mixture was stirred at 80° C. for 12 hours before adding again brominated derivative (4eq) and potassium carbonate (4eq). The reaction mixture was stirred at 80° C. another 20 hours. The dimethylformamide was evaporated under reduced pressure. The residue was partitioned between ethyl acetate and water. The aqueous layer was washed with ethyl acetate. The combined organic layers were dried over sodium sulfate, filtered, and evaporated under reduced pressure. The residue was chromatographed over silica gel.

Methyl Bromination

To a solution of the biphenylmethyl derivative previously prepared (1eq) in carbon tetrachloride (80eq) were added N-bromosuccinimide (1.2eq) and 2.2′-azo-bis-isobutyronitrile (AIBN) (0.015eq). The reaction mixture was stirred at 80° C. for 15 minutes then AIBN (0.016) was added. The mixture was stirred at reflux for 12 hours. The reaction mixture was cooled to room temperature. The resulting precipitate was filtered and the filtrate was evaporated under reduced pressure. The residue was taken up in dichloromethane and washed with a saturated sodium thiosulfate aqueous solution then with brine. The combined organic layers were dried over sodium sulfate, filtered, and evaporated under reduced pressure. The residue was chromatographed over silica gel.

Method 6C: using the appropriate hydroxyphenylboronic acid. The Suzuki reaction was followed by O-alkylation. The aromatic methyl was then free-radical brominated.

Suzuki Reaction

To a solution of bromotoluene (1eq) in dioxane (30eq) were added the appropriate hydroxyphenylboronic acid (1.1eq), the tetrakis(triphenylphosphine)palladium (Pd[P(Ph)₃]₄) derivative (0.03eq) and potassium carbonate (3eq). The reaction mixture was stirred at 100° C. for 16 hours. After cooling down, the solvent was evaporated under reduced pressure. The residue was taken up in ethyl acetate and washed with brine. The organic layer was dried over sodium sulfate, filtered, and concentrated under reduced pressure. The residue was chromatographed over silica gel.

Phenol Substitution

To a solution of the 4′-methylbiphenol previously prepared (1eq) in dimethylformamide, was added potassium carbonate (4eq). The suspension was stirred at 80° C. The halogenated derivative was then added drop by drop and the reaction mixture was stirred at 80° C. for 48 hours. The potassium carbonate was filtered and the dimethylformamide was evaporated under reduced pressure. The residue was taken up in ethyl acetate and washed with brine. The aqueous layer was dried over sodium sulfate, filtered, and evaporated under reduced pressure. The residue was chromatographed over silica gel.

Methyl Bromination

To a solution of the biphenylmethyl derivative previously prepared (1eq) in carbon tetrachloride (80eq) were added N-bromosuccinimide (0.95eq) and 2.2′-azo-bis-isobutyronitrile (AIBN) (0.5eq). The reaction mixture was stirred at 80° C. for 6 hours. The reaction mixture was cooled to room temperature. The resulting precipitate was filtered and the filtrate was evaporated under reduced pressure. The residue was taken up in dichloromethane and washed with a saturated sodium thiosulfate aqueous solution and with brine. The organic layer was dried over sodium sulfate, filtered, and concentrated under reduced pressure. The residue was chromatographed over silica gel.

Method 6D: using the appropriate 1,2,4-triazole-3-thiol. The thiazolotriale with an ester function was prepared. The cyclisation was followed by a reduction of the ester function. The hydroxyl group was then brominated with N-bromosuccinimide and triphenylphosphine.

Cyclisation of 1,2.4-triazole-3-thiol in thiazolotriazole

To a solution of the 1,2,4-triazole-3-thiol (1eq) in anhydrous ethanol was added ethyl 2-chloroacetoacetate drop by drop at room temperature. The reaction mixture was stirred at reflux for 12 hours. The resulting precipitate was filtered, washed with ethanol and dried in a desiccator.

Ester reduction

The ester previously prepared was dissolved in anhydrous THF. The solution was cooled in an ice bath. Lithium tetrahydroaluminate was the added in portions. The reaction mixture was stirred for 2 hours. After adding water, the sodium hydroxide 2N solution then water, the reaction mixture as stirred for 15 minutes the filtered. The filtrate was evaporated under reduced pressure. The residu was recristallized in acetonitrile.

Preparation of the Brominated Derivative

To a suspension of the alcohol previously prepared (1eq) in acetonitrile and the suspension at 0° C. was added triphenylphosphine (3eq) in portions. After 5 minutes of stirring, N-bromosuccinimide (3eq) was added in portions at 0° C. The reaction mixture was stirred at room temperature for 12 hours, then evaporated under reduced pressure. The residue was taken up in a minimal amount of dichloromethane and purified by filtration on silica gel.

### Example 6.1

### ethyl 2-((4′-bromomethylbiphenyl-2-yl)oxy)-2-methylpropanoate and ethyl 2-((5-bromo-4′-bromomethyl biphenyl-2-yl)oxy)-2-methylpropanoate

### 6.1.1 ethyl 2-(2-bromophenyloxy)-2-methylpropanoate

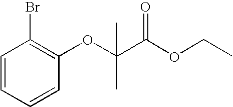

Prepared following the general substitution procedure previously described (Method 6A) using 2-bromophenol and ethyl 2-bromoisobutyrate. The product was chromatographed over silica gel (eluent cyclohexane/ethyl acetate 95/5). The product was obtained as a colorless oil.

Yield: 47%

Rf (cyclohexane/ethyl acetate 9/1): 0.55

IR: νCO: 1734 cm⁻¹

NMR ¹H (CDCl₃): 1.27 (t, 3H, J=7 Hz); 1.63 (s, 6H); 4.26 (q, 2H, J=7 Hz); 6.84-6.89 (m, 2H); 7.17 (td, 1H, J=6.7 Hz, J=1.5 Hz); 7.54 (dd, 1H, J=6.7 Hz, J=1.5 Hz).

### 6.1.2 ethyl 2-((4′-methylbiphenyl-2-yl)oxy)-2-methylpropanoate

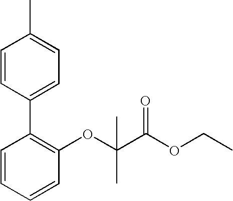

Prepared following the Suzuki reaction previously described (Method 6A) using ethyl 2-(2-bromophenyloxy)-2-methylpropanoate (example 6.1.1) and 4-tolyboronic acid. The product was chromatographed over silica gel (eluent cyclohexane/dichloromethane 7/3). The product was obtained as a colorless oil.

Yield: 58%

Rf (cyclohexane/dichloromethane 7/3): 0.30

IR: νCO: 1735 cm⁻¹

NMR ¹H (CDCl₃): 1.27 (t, 3H, J=7 Hz); 1.44 (s, 6H); 2.41 (s, 3H); 4.25 (q, 2H, J=7.3 Hz); 6.89 (d, 1H, J=8.2 Hz); 7.08 (t, 1H, J=7.3 Hz); 7.23 (m, 3H); 7.35 (dd, 1H, J=7.3 Hz, J=1.5 Hz,); 7.49 (d, 2H, J=8.2 Hz).

### 6.1.3 ethyl 2-((4′-bromomethylbiphenyl-2-yl)oxy)-2-methylpropanoate and ethyl 2-((5-bromo-4′-bromomethylbiphenyl-2-yl)oxy)-2-methylpropanoate

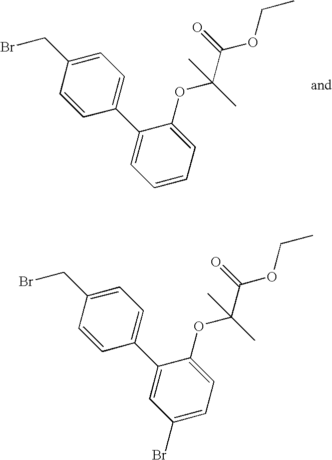

Prepared according to the bromination reaction previously described (Method 6A) using ethyl 2-((4′-methylbiphenyl-2-yl)oxy)-2-methylpropanoate (example 6.1.2). The products were chromatographed over silica gel (eluent cyclohexane/ethyl acetate 95/5). The products were obtained as a colorless oil (mixture of two compounds).

Total yield: 61%

Rf (cyclohexane/ethyl acetate 8/2): 0.70

IR: νCO: 1733 cm⁻¹

NMR ¹H (CDCl₃) (derivative non-brominated on the aromatic cycle): 1.26 (t, 3H, J=7 Hz); 1.65 (s, 6H); 4.24 (q, 2H, J=7 Hz); 4.56 (s, 2H); 6.87 (d, 2H, J=7.6 Hz); 7.08 (t, 1H, J=7.3 Hz); 7.15-7.25 (m, 2H); 7.33 (dd, 1H, J=7.6 Hz, J=1.8 Hz); 7.55 (d, 2H, J=7.6 Hz).

NMR ¹H (CDCl₃) (derivative brominated on the aromatic cycle): 1.29 (t, 3H, J=7 Hz); 1.65 (s, 6H); 4.29 (q, 2H, J=7 Hz); 4.57 (s, 2H); 6.78 (dd, 1H, J=8.2 Hz, J=2.9 Hz); 6.88 (d, 1H, J=2.9 Hz); 7.17 (d, 2H, J=8 Hz); 7.33 (d, 2H, J=7.6 Hz); 7.50 (d, 1H, J=7.6 Hz).

### Example 6.2

### ethyl 2-((4′-bromomethylbiphenyl-3-yl)oxy)-2-methylpropanoate and ethyl 2-((6-bromo-4′-bromomethyl biphenyl-3-yl)oxy)-2-methyl propanoate

### 6.2.1 ethyl 2-(3-bromophenyloxy)-2-methylpropanoate

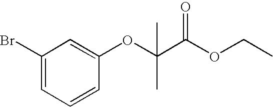

Prepared following the general substitution procedure previously described (Method 6A) using 3-bromophenol and ethyl 2-bromoisobutyrate. The product was chromatographed over silica gel (eluent cyclohexane/ethyl acetate 9/1). The product was obtained as a colorless oil.

Yield: 85%

Rf (cyclohexane/ethyl acetate 9/1): 0.50

IR: νCO: 1736 cm⁻¹

NMR ¹H (CDCl₃): 1.26 (t, 3H, J=7.3 Hz); 1.61 (s, 6H); 4.25 (q, 2H, J=7.3 Hz); 6.77 (d, 1H, J=7 Hz); 7.03 (s, 1H); 7.08 (m, 2H).

### 6.2.2 ethyl 2-((4′-methylbiphenyl-3-yl)oxy)-2-methyl propanoate

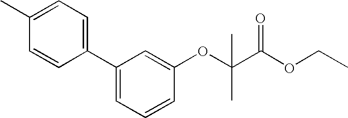

Prepared following the Suzuki reaction previously described (Method 6A) using ethyl 2-(3-bromophenyloxy)-2-methylpropanoate (example 6.2.1) and 4-tolyboronic acid. The product was chromatographed over silica gel (eluent cyclohexane/dichloromethane 8/2, then toluene/cyclohexane 7/3). The product was obtained as a colorless oil.

Yield: 41%

Rf (cyclohexane/dichloromethane 7/3): 0.30

IR: νCO: 1733 cm⁻¹

NMR ¹H (CDCl₃): 1.28 (t, 3H, J=7.3 Hz); 1.66 (s, 6H); 2.42 (s, 3H); 4.27 (q, 2H, J=7.3 Hz); 6.81 (m, 1H); 7.13 (m, 1H); 7.22-7.33 (m, 4H); 7.48 (d, 2H, J=8.2 Hz).

### 6.2.3 ethyl 2-((4′-bromomethylbiphenyl-3-yl)oxy)-2-methylpropanoate and ethyl 2-((6-bromo-4′-bromomethylbiphenyl-3-yl)oxy)-2-methylpropanoate

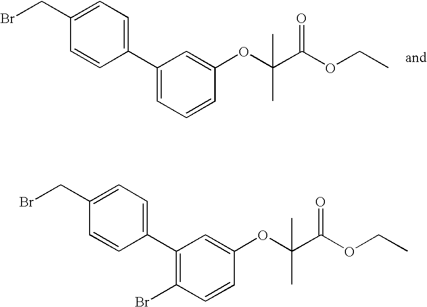

Prepared following the bromination reaction previously described (Method 6A) using ethyl 2-((4′-methylbiphenyl-3-yl)oxy)-2-methylpropanoate (example 6.2.2). The products were chromatographed over silica gel (eluent cyclohexane/acetone 97/3). The products were obtained as a colorless oil (mixture of two compounds).

Total yield: 57%

Rf (cyclohexane/acetone 97/3): 0.25

IR: νCO: 1732 cm⁻¹

NMR ¹H (CDCl₃) (derivative non-brominated on the aromatic cycle): 1.26 (t, 3H, J=7 Hz); 1.66 (s, 6H); 4.27 (q, 2H, J=7 Hz); 4.56 (s, 2H); 6.81-6.86 (dd, 1H, J=8.2 Hz, J=2.6 Hz); 7.12 (t, 1H, J=1.7 Hz); 7.21-7.26 (td, 1H, J=6.5 Hz, J=1.4 Hz); 7.30-7.35 (m, 1H); 7.46 (d, 2H, J=8.5 Hz); 7.55 (d, 2H, J=8.2 Hz).

NMR ¹H (CDCl₃) (derivative brominated on the aromatic cycle): 1.29 (t, 3H, J=7 Hz); 1.62 (s, 6H); 4.21-4.30 (m, 4H); 6.67-6.73 (dd, 1H, J=8.8 Hz, J=2.9 Hz); 6.86 (d, 1H, J=2.9 Hz); 7.10-7.14 (d, 1H, J=7 Hz); 7.26-7.32 (d, 2H, J=8.5 Hz); 7.48-7.53 (d, 2H, J=8.8 Hz).

### Example 6.3

### ethyl 2-((4′-bromomethylbiphenyl-4-yl)oxy)-2-methylpropanoate

### 6.3.1 ethyl 2-(4-bromophenyloxy)-2-methylpropanoate

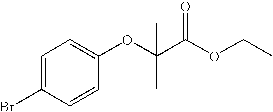

Prepared following the general substitution procedure previously described (Method 6A) using 4-bromophenol and ethyl 2-bromoisobutyrate. The product was chromatographed over silica gel (eluent cyclohexane/ethyl acetate 95/5). The product was obtained as a colorless oil.

Yield: 60%

Rf (cyclohexane/ethyl acetate 9/1): 0.55

IR: νCO: 1734 cm⁻¹

NMR ¹H (CDCl₃): 1.24 (t, 3H, J=7.1 Hz); 1.58 (s, 6H); 4.19-4.26 (q, 2H, J=7.5 Hz); 6.73 (d, 2H, J=9.1 Hz); 7.33 (d, 2H, J=9.1 Hz).

### 6.3.2 ethyl 2-((4′-methylbiphenyl-4-yl)oxy)-2-methylpropanoate

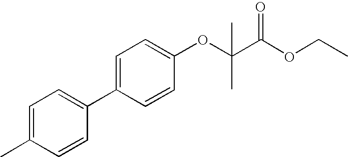

Prepared following the Suzuki reaction previously described (Method 6A) using ethyl 2-(4-bromophenyloxy)-2-methylpropanoate (example 6.3.1) and 4-tolyboronic acid. The product was chromatographed over silica gel (eluent cyclohexane/dichloromethane 7/3). The product was obtained as a colorless oil.

Yield: 61%

Rf (cyclohexane/dichloromethane 6/4): 0.30

IR: νCO: 1722 cm⁻¹

NMR ¹H (CDCl₃): 1.27 (t, 3H, J=7.1 Hz); 1.67 (s, 6H); 2.41 (s, 3H); 4.25-4.32 (q, 2H, J=7.1 Hz); 6.93-6.96 (d, 2H, J=8.7 Hz); 7.24-7.26 (d, 2H, J=7.7 Hz); 7.45-7.49 (d, 2H, J=8.2 Hz); 7.48-7.53 (d, 2H, J=8.2 Hz).

### 6.3.3 ethyl 2-((4′-bromomethylbiphenyl-4-yl )oxy)-2-methylpropanoate

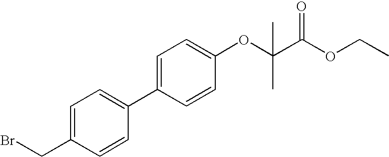

Prepared following the bromination reaction previously described (Method 6A) using ethyl 2-((4′-methylbiphenyl-4-yl)oxy)-2-methylpropanoate (example 6.3.2).

The product was chromatographed over silica gel (eluent cyclohexane/ethyl acetate 95/5). The product was obtained as a colorless oil.

Yield: 58%

Rf (cyclohexane/dichloromethane 6/4): 0.35

IR: νCO: 1730 cm⁻¹

NMR ¹H (CDCl₃): 1.26 (t, 3H, J=7 Hz); 1.66 (s, 6H); 4.26-4.33 (q, 2H, J=7 Hz); 4.56 (s, 2H); 6.93-6.96 (d, 2H, J=8.7 Hz); 7.24-7.26 (d, 2H, J=7.7 Hz); 7.45-7.49 (d, 2H, J=8.2 Hz); 7.48-7.53 (d, 2H, J=8.2 Hz).

### Example 6.4

### tert-butyl 2-((4′-bromomethylbiphenyl-3-yl)oxy)-2-methyl propanoate

### 6.4.1 4′-methylbiphenyl-3-ol

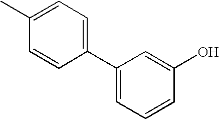

Prepared following the Suzuki condensation method previously described (Method 6B) using 3-bromophenol and 4-tolylboronic acid. The product was chromatographed over silica gel (eluent cyclohexane, and then cyclohexane/ethyl acetate 95/5, then 9/1). The product was obtained as a brown oil.

Yield: 77%

Rf (cyclohexane/ethyl acetate 9/1): 0.30

NMR ¹H (CDCl₃): 2.44 (s, 3H); 6.82 (d, 1H, J=8.5 Hz); 7.08 (s, 1H); 7.18 (d, 1H, J=8 Hz); 7.27 (d, 2H, J=8.2 Hz); 7.31 (t, 1H, J=8 Hz); 7.50 (d, 2H, J=8.2Hz).

### 6.4.2 tert-butyl 2-((4′-methylbiphenyl-3-yl)oxy)-2-methylpropanoate

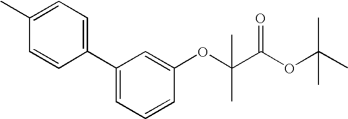

Prepared following the alkylation reaction previously described (Method 6B) using 4′-methylbiphenyl-3-ol (example 6.4.1) and tert-butyl 2-bromoisobutyrate. The product was chromatographed over silica gel (eluent cyclohexane, and then cyclohexane/ethyl acetate 98/2, then 99/1). The product was obtained as a colorless oil.

Yield: 40%

NMR ¹H (CDCl₃): 1.51 (s, 9H); 1.66 (s, 6H); 2.44 (s, 3H); 6.88 (d, 1H, J=8.5 Hz); 7.17 (s, 1H); 7.21-7.38 (m, 4H); 7.52 (d, 2H, J=8.2 Hz).

### 6.4.3 tert-butyl 2-((4′-bromomethylbiphenyl-3-yl)oxy)-2-methylpropanoate

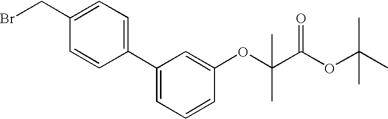

Prepared following the bromination reaction previously described (Method 6B) using tert-butyl 2-((4′-methylbiphenyl-3-yl)oxy)-2-methylpropanoate (example 6.4.2). After extraction the productobtained as a colorless oil, was used without any further purification.

Yield: 90%

NMR ¹H (CDCl₃): 1.45 (s, 9H); 1.62 (s, 6H); 4.56 (s, 2H); 6.88 (dd, 1H, J=8.2 Hz, J=2.6 Hz); 7.14 (s, 1H); 7.21-7.26 (d, 1H, J=6.5 Hz); 7.30-7.35 (t, 1H, J=7 Hz); 7.48 (d, 2H, J=8.5 Hz); 7.56 (d, 2H, J=8.5 Hz).

### Example 6.5

### 2-((4′-bromomethylbiphenyl-3-yl)oxy)acetonitrile

### 6.5.1 2-(3-bromophenoxy)acetonitrile

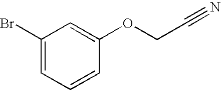

Prepared following the general substitution procedure previously described (Method 6A) using 3-bromophenol and 2-chloroacetonitrile. The product was chromatographed over silica gel (eluent cyclohexane/dichloromethane 5/5). The product was obtained as a colorless oil. Yield: 93%

Rf (dichloromethane/ethyl acetate 98/2): 0.70

IR: νCC: 1589 cm⁻¹

NMR ¹H (CDCl₃): 4.76 (s, 2H); 6.92-6.95 (m, 1H); 7.16 (s, 1H); 7.22-7.24 (m, 2H).

### 6.5.2 2-((4′-methylbiphenyl-3-yl)oxy)acetonitrile

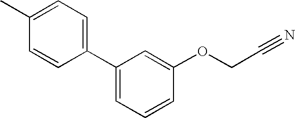

Prepared following the Suzuki reaction previously described (Method 6A) using ethyl 2-(3-bromophenyloxy)acetonitrile (example 6.5.1) and 4-tolyboronic acid. The product was chromatographed over silica gel (eluent cyclohexane/ethyl acetate 85/15). The product was obtained as a colorless oil.

Yield: 70%

Rf (cyclohexane/ethyl acetate 8/2): 0.50

IR: νCC: 1588 cm⁻¹

NMR ¹H (CDCl₃): 2.43 (s, 3H); 4.83 (s, 2H); 6.90-7.00 (m, 1H); 7.17 (d, 1H, J=1.8 Hz); 7.24-7.36 (m, 3H); 7.40-7.45 (t,1H, J=7.9 Hz); 7.50 (d, 2H, J=7.9 Hz).

### 6.5.3 2-((4′-bromomethylbiphenyl-3-yl)oxy)acetonitrile

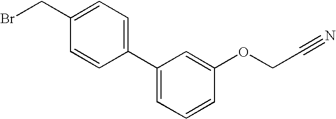

Prepared following the bromination reaction previously described (Method 6A) using 2-((4′-methylbiphenyl-3-yl)oxy)acetonitrile (example 6.5.2). The product was chromatographed over silica gel (eluent cyclohexane/ethyl acetate 9/1). The product was obtained as a white solid.

Yield: 16%

Rf (cyclohexane/ethyl acetate 8/2): 0.80

IR: νCC: 1588 cm⁻¹

NMR ¹H (CDCl₃): 4.57 (s, 2H); 4.85 (s, 2H); 6.98-7.03 (ddd, 1H, J=8.2 Hz, J=2.6 Hz, J=0.9 Hz); 7.15-7.21 (dd, 1H, J=9.1 Hz, J=2.6 Hz); 7.30-7.36 (dd, 1H, J=6.4 Hz, J=1.2 Hz); 7.42-7.47 (t, 1H, J=7.9 Hz); 7.47-7.52 (d, 2H, J=8.5 Hz); 7.55-7.60 (d, 2H, J=8.2 Hz).

### Example 6.6

### methyl 5-((4′-bromomethylbiphenyl-4-yl)oxy)-2,2-dimethyl-pentanoate

### 6.6.1 4′-methylbiphenyl-4-ol

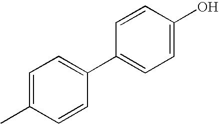

Prepared following the Suzuki reaction previously described (Method 6C) using 4-bromotoluene and 4-hydroxyphenylboronic acid. The product was chromatographed over silica gel (eluent cyclohexane/ethyl acetate 9/1). The product was obtained as a pale yellow solid.

Yield: 49%

Rf (cyclohexane/ethyl acetate 8/2): 0.37

NMR ¹H (CDCl₃): 2.44 (s, 3H); 4.83 (s, 1H); 6.94 (d, 2H, J=8 Hz); 7.30 (d, 2H J=8 Hz); 7.50 (t, 4H, J=8 Hz)

### 6.6.2

### methyl 2,2-dimethyl-5-((4′-methylbiphenyl-4-yl )oxy)pentanoate

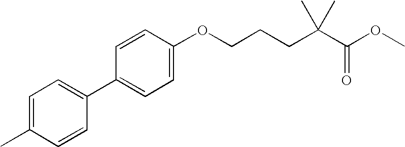

Prepared following the general substitution procedure previously described (Method 6C) using 4′-methylbiphenyl-4-ol (example 6.6.1) and methyl 2,2-dimethyl-5-iodo-pentanoate (example 4.1). The product was chromatographed over silica gel (eluent cyclohexane/ethyl acetate 9/1). The product was obtained as a yellow oil.

Yield: 82%

Rf (cyclohexane/ethyl acetate 8/2): 0.56

NMR ¹H (CDCl₃): 1.30 (s, 6H); 1.79 (m, 4H); 2.43 (s, 3H); 3.72 (s, 3H); 4.02 (t, 2H, J=6 Hz); 6.99 (d, 2H, J=8 Hz); 7.29 (t, 2H, J=8 Hz); 7.50 (d, 2H, J=8 Hz); J=8 Hz).

### 6.6.3 methyl 5-((4′-bromomethylbiphenyl-4-yl)oxy)-2,2-dimethyl-pentanoate

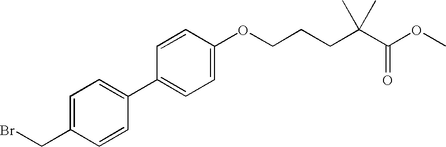

Prepared following the bromination reaction previously described (Method 6C) using methyl 2,2-dimethyl-5-((4′-methylbiphenyl-4-yl)oxy)pentanoate (example 6.6.2). The product was chromatographed over silica gel (eluent cyclohexane/ethyl acetate 9/1). The product was obtained as a white solid.

Yield: 61%

Rf (cyclohexane/ethyl acetate 8/2): 0.45

NMR ¹H (CDCl₃): 1.25 (s, 6H); 1.76 (m, 4H); 3.70 (s, 3H); 4.00 (t, 2H, J=6 Hz); 4.56 (s, 2H); 6.98 (d, 2H, J=8 Hz); 7.46 (d, 2H, J=8 Hz); 7.53 (t, 4H, J=8 Hz).

### Example 6.7

### methyl 5-((4′-bromomethylbiphenyl-3-yl)oxy)-2,2-dimethyl-pentanoate

### 6.7.1 methyl 2,2-dimethyl-5-(4′-methyl-biphenyl-3-yloxy)pentanoate

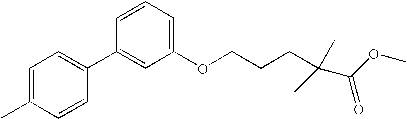

Prepared following the general substitution procedure previously described (Method 6C) using 4′-methylbiphenyl-3-ol (example 6.4.1) and methyl 2,2-dimethyl-5-iodo-pentanoate (example 4.1). The product was chromatographed over silica gel (eluent cyclohexane/ethyl acetate 98/2). The product was obtained as a yellow oil.

Yield: 82%

Rf (cyclohexane/ethyl acetate 8/2): 0.57

NMR ¹H (CDCl₃): 1.30 (s, 6H); 1.72 (m, 4H); 2.40 (s, 3H); 3.68 (s, 3H); 3.99 (t, 2H, J=6 Hz); 6.85 (dd, 1H, J=8.5 Hz; J=2 Hz); 7.10 (t, 1H, J=2 Hz); 7.20-7.38 (m, 4H); 7.49 (d, 2H, J=8.2 Hz).

### 6.7.2 methyl 5-((4′-bromomethylbiphenyl-3-yl )oxy)-2,2-dimethyl-pentanoate

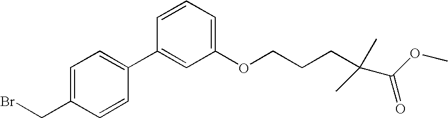

Prepared following the bromination reaction previously described (Method 6C) using methyl 2,2-dimethyl-5-((4′-methylbiphenyl-3-yl)oxy)pentanoate (example 6.7.1). The product was chromatographed over silica gel (eluent cyclohexane, and then cyclohexane/ethyl acetate 99/1, then 98/2). The product was obtained as a colorless oil.

Yield: 50%

Rf (cyclohexane/ethyl acetate 8/2): 0.44

NMR ¹H (CDCl₃): 1.25 (s, 6H); 1.40-1.55 (m, 2H); 1.65-1.80 (m, 2H); 3.65 (s, 3H); 4.00 (t, 2H, J=6 Hz); 4.53 (s, 2H); 6.88 (dd, 1H, J=8.5 Hz ; J=2 Hz); 7.10 (t, 1H, J=2 Hz); 7.20-7.38 (m, 4H); 7.49 (d, 2H, J=8.2 Hz).

### Example 6.8.

### methyl 5-((4′-bromomethylbiphenyl-2-yl)oxy)-2,2-dimethyl-pentanoate

### 6.8.1

### 4′-methylbiphenyl-2-ol

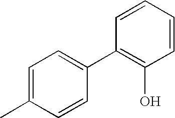

Prepared following the Suzuki reaction previously described (Method 6C) using 4-bromotoluene and 2-hydroxyphenylboronic acid. The product was chromatographed over silica gel (eluent cyclohexane, and then cyclohexane/ethyl acetate 95/5, then 9/1). The product was obtained as an oil.

Yield: 86%

Rf (cyclohexane/ethyl acetate 8/2): 0.55

NMR ¹H (CDCl₃): 2.40 (s, 3H); 5.25 (s, 1H); 6.99 (m, 2H); 7.18-7.50 (m, 6H).

### 6.8.2

### methyl 2,2-dimethyl-5-((4′-methylbiphenyl-2-yl)oxy)pentanoate

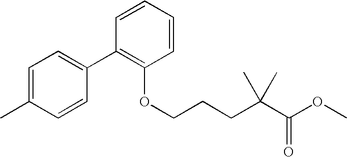

Prepared following the general substitution procedure previously described (Method 6C) using 4′-methylbiphenyl-2-ol (example 6.8.1) and methyl 2,2-dimethyl-5-iodo-pentanoate (example 4.1). The product was chromatographed over silica gel (eluent cyclohexane/ethyl acetate 98/2, then 95/5). The product was obtained as a yellow oil.

Yield: 69%

Rf (cyclohexane/ethyl acetate 8/2): 0.55

NMR ¹H (CDCl₃): 1.15 (s, 6H); 1.63 (m, 4H); 2.40 (s, 3H); 3.65 (s, 3H); 3.92 (t, 2H, J=6 Hz); 6.99 (m, 2H); 7.15-7.38 (m, 4H); 7.45 (d, 2H, J=8 Hz).

### 6.8.3

### methyl 5-((4′-bromomethylbiphenyl-2-yl)oxy)-2,2-dimethyl-pentanoate

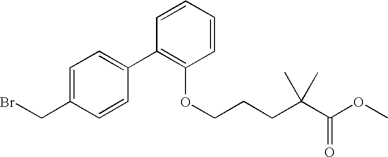

Prepared following the bromination reaction previously described (Method 6C) using methyl 2,2-dimethyl-5-((4′-methylbiphenyl-2-yl)oxy)pentanoate (example 6.8.2). The product was chromatographed over silica gel (eluent cyclohexane/ethyl acetate 100/0 to 96/4). The product was obtained as a yellow oil.

Yield: 49%

Rf (cyclohexane/ethyl acetate 8/2): 0.45

NMR ¹H (CDCl₃): 1.15 (s, 6H); 1.62 (m, 4H); 3.60 (s, 3H); 3.95 (t, 2H, J=6 Hz); 4.54 (s, 2H); 6.99 (m, 2H); 7.20-7.45 (m, 4H); 7.55 (m, 2H).

### Example 6.9

### methyl 8-((4′-bromomethylbiphenyl-2-yl)oxy)-2,2-dimethyl-octanoate

### 6.9.1

### methyl 2,2-dimethyl-8-((4′-methylbiphenyl-2-yl)oxy)octanoate

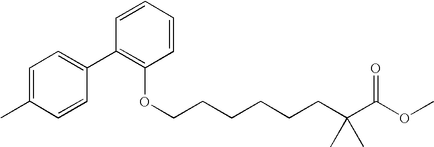

Prepared following the general substitution procedure previously described (Method 6C) using 4′-methylbiphenyl-2-ol (example 6.8.1) and methyl 2,2-dimethyl-8-iodo-octanoate (example 4.2). The product was chromatographed over silica gel (eluent cyclohexane/ethyl acetate 98/2, then 95/5). The product was obtained as a yellow oil.

Yield: 85%

Rf (cyclohexane/ethyl acetate 8/2): 0.55

NMR ¹H (CDCl₃): 1.15 (s, 6H); 1.20-1.30 (m, 2H); 1.42-1.55 (m, 6H); 1.70 (t, 2H, J=7 Hz); 2.39 (s, 3H); 3.65 (s, 3H); 3.95 (t, 2H, J=6 Hz); 6.99 (m, 2H); 7.15-7.38 (m, 4H); 7.44 (d, 2H, J=8 Hz).

### 6.9.2

### methyl 8-((4′-bromomethylbiphenyl-2-yl)oxy)-2,2-dimethyl-octanoate

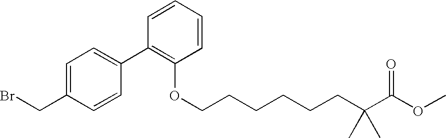

Prepared following the bromination reaction previously described (Method 6C) using methyl 2,2-dimethyl-8-((4′-methylbiphenyl-2-yl)oxy)octanoate (example 6.9.1). The product was chromatographed over silica gel (eluent cyclohexane, and then cyclohexane/ethyl acetate 98/2, then 96/4). The product was obtained as a yellow oil.

Yield: 48%

Rf (cyclohexane/ethyl acetate 8/2): 0.45

NMR ¹H (CDCl₃): 1.15 (s, 6H); 1.18-1.59 (m, 8H); 1.70 (quint, 2H, J=7 Hz); 3.64 (s, 3H); 3.95 (t, 2H, J=6 Hz); 4.55 (s, 2H); 6.99 (m, 2H); 7.15-7.38 (m, 4H); 7.44 (d, 2H, J=8 Hz).

### Example 6.10.

### methyl 3-((4′-bromomethylbiphenyl-3-yl)oxy)-2,2-dimethyl-propanoate

### 6.10.1

### methyl 2,2-dimethyl-3-((4′-methylbiphenyl-3-yl)oxy)propanoate

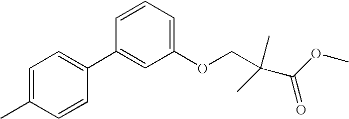

Prepared following the general substitution procedure previously described (Method 6C) using 4′-methylbiphenyl-3-ol (example 6.4.1) and methyl 2,2-dimethyl-3-iodo-propanoate (example 4.3). The product was chromatographed over silica gel (eluent cyclohexane/ethyl acetate 98/2). The product was obtained as a yellow oil.

Yield: 70%

Rf (cyclohexane/ethyl acetate 8/2): 0.50

NMR ¹H (CDCl₃): 1.20 (s, 6H); 2.38 (s, 3H); 3.53 (s, 3H); 3.95 (s, 2H); 6.95 (m, 2H); 7.10-7.50 (m, 6H).

### 6.10.2

### methyl 3-((4′-bromomethylbiphenyl-3-yl)oxy)-2,2-dimethyl-propanoate

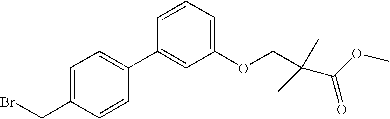

Prepared following the bromination reaction previously described (Method 6C) using methyl 2,2-dimethyl-3-((4′-methylbiphenyl-3-yl)oxy)propanoate (example 6.10.1). The product was chromatographed over silica gel (eluent cyclohexane/ethyl acetate 98/2, then 95/5). The product was obtained as a yellowish oil.

Yield: 74%

Rf (cyclohexane/ethyl acetate 8/2): 0.46

NMR ¹H (CDCl₃): 1.15 (s, 6H); 3.55 (s, 3H); 3.95 (s, 2H); 4.58 (s, 2H); 7.00 (m, 2H); 7.20-7.50 (m, 6H).

### Example 6.11.

### 5-bromomethyl-2-(4-methoxyphenyl)-6-methyl-thiazolo[3,2-b][1,2,4]triazole

### 6.11.1

### 5-ethoxycarbonyl-2-(4-methoxyphenyl)-6-methyl-thiazolo[3,2-b][1,2,4]triazole

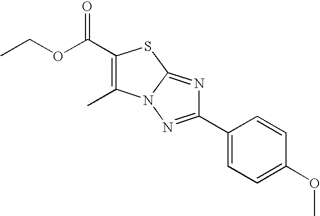

Prepared following the cyclisation reaction previously described (Method 6D) using 5-(4-methoxyphenyl)-2H-1,2,4-triazole-3-thiol and ethyl chloroacetoacetate. The compound was obtained as a white solid.

Yield: 52%

Rf (cyclohexane/ethyl acetate 70/30): 0.6

IR: νCO: 1706 cm⁻¹

NMR ¹H (DMSO): 1.33 (t, 3H, J=7.3 Hz); 2.84 (s, 3H); 3.82 (s, 3H); 4.35 (q, 2H, J=7.3 Hz); 7.05 (d, 2H, J=8.8 Hz); 8.02 (d, 2H, J=8.8 Hz).

### 6.11.2

### 5-hydroxymethyl-2-(4-methoxyphenyl)-6-methyl-thiazolo[3,2-b][1,2,4]triazole

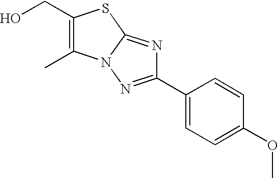

Prepared following the reduction reaction previously described (Method 6D) using 5-ethoxycarbonyl-2-(4-methoxyphenyl)-6-methyl-thiazolo[3,2-b][1,2,4]triazole (example 6.11.1). The product was purified by recristallization in acetonitrile. The product was obtained as a white solid.

Yield: 34%

Rf (cyclohexane/ethyl acetate 60/40): 0.2

NMR ¹H (Methanol-d4): 2.55 (s, 3H); 3.86 (s, 3H); 4.76 (s, 2H); 7.01 (d, 2H, J=8.8 Hz); 8.03 (d, 2H, J=8.8 Hz).

### 6.11.3

### 5-bromomethyl-2-(4-methoxyphenyl)-6-methyl-thiazolo[3,2-b][1,2,4]triazole

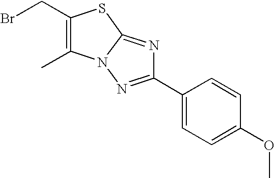

Prepared following the bromination reaction previously described (Method 6D) using 5-hydroxymethyl-2-(4-methoxyphenyl)-6-methyl-thiazolo[3,2-b][1,2,4]triazole (example 6.11.2). The product was chromatographed over silica gel (eluent cyclohexane/ethyl acetate 70/30). The product was obtained as a yellowish solid.

Yield: 35%

Rf (cyclohexane/ethyl acetate 70/30): 0.4

NMR ¹H (DMSO): 2.53 (s, 3H); 3.82 (s, 3H); 5.15 (s, 2H); 7.05 (d, 2H, J=8.8 Hz); 8.00 (d, 2H, J=8.8 Hz).

### Example 6.12.

### 5-bromomethyl-2-(3-methoxyphenyl)-6-methyl-thiazolo[3,2-b][1,2,4]triazole

### 6.12.1

### 5-ethoxycarbonyl-2-(3-methoxyphenyl)-6-methyl-thiazolo[3,2-b][1,2,4]triazole

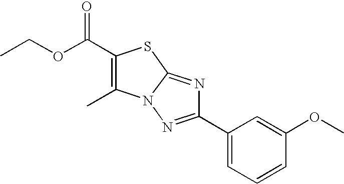

Prepared following the cyclisation reaction previously described (Method 6D) using 5-(3-methoxyphenyl)-2H-1,2,4-triazole-3-thiol and ethyl chloroacetoacetate. The compound was obtained as a white solid.

Yield: 33.3%

Rf (cyclohexane/ethyl acetate 70/30): 0.5

IR: νCO: 1694 cm⁻¹

NMR ¹H (DMSO): 1.43 (t, 3H, J=7.2 Hz); 2.95 (s, 3H); 3.91 (s, 3H); 4.41 (q, 2H, J=7.2 Hz); 7.01 (m, 1H); 7.39 (m, 1H); 7.72 (m, 1H); 7.80 (d, 2H, J=7.6 Hz).

### 6.12.2

### 5-hydroxymethyl-2-(3-methoxyphenyl)-6-methyl-thiazolo[3,2-b][1,2,4]triazole

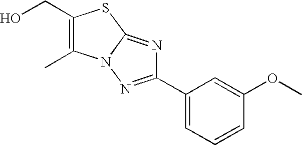

Prepared following the reduction reaction previously described (Method 6D) using 5-ethoxycarbonyl-2-(3-methoxyphenyl)-6-methyl-thiazolo[3,2-b][1,2,4]triazole (example 6.12.1). The product was chromatographed over silica gel (eluent dichloromethane/methanol 98/2). The product was obtained as a white solid.

Yield: 55.4%

Rf (cyclohexane/ethyl acetate 70/30): 0.15

NMR ¹H (CDCl₃): 2.53 (s, 3H); 3.91 (s, 3H); 4.78 (s, 2H); 6.98 (m, 1H); 7.37 (m, 1H); 7.27 (m, 1H); 7.78 (m, 1H).

### 6.12.3

### 5-bromomethyl-2-(3-methoxyphenyl)-6-methyl-thiazolo[3,2-b][1,2,4]triazole

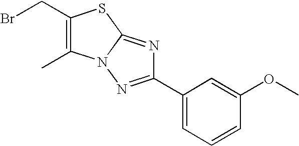

Prepared following the cyclisation reaction previously described (Method 6D) using 5-hydroxymethyl-2-(3-methoxyphenyl)-6-methyl-thiazolo[3,2-b][1,2,4]triazole (example 6.12.2). The product was chromatographed over silica gel (eluent cyclohexane/ethyl acetate 70/30). The product was obtained as a yellowish solid.

Yield: 31%

Rf (cyclohexane/ethyl acetate 70/30): 0.5

NMR ¹H (CDCl₃): 2.60 (s, 3H); 3.91 (s, 3H); 4.67 (s, 2H); 7.00 (m, 1H); 7.39 (m, 1H); 7.72 (m, 1H); 7.78 (m, 1H).

### Example 7.

### General Procedure for the Preparation of Benzoylbenzyl Bromides

Benzoylbenzyl bromides were prepared in 3 or 4 steps using toluene and the appropriate methoxybenzoyl chloride. Friedel-Crafts acylation was followed by demethylation of the methoxy function, then by O-alkylation. The aromatic methyl was then free-radical brominated.

Friedel-Crafts Acylation

To a solution of aluminium chloride (1.1eq) in toluene (10eq) at 0° C. was added the appropriate acyl chloride (1eq) drop by drop. The reaction mixture was stirred to room temperature for 12 hours. The reaction mixture was then slowly hydrolysed by addition of water, and then extracted with ethyl acetate. The combined organic layers were dried over magnesium sulfate, and concentrated under reduced pressure. The residue was chromatographed over silica gel.

Demethylation

To a solution of the methoxy derivative previously prepared in chloroform was added at 0° C. boron tribromide (2eq) drop by drop. The reaction mixture was stirred at room temperature for 24-48 hours. The mixture was then partitioned between water and dichloromethane. The organic layers were dried over magnesium sulfate and evaporated under reduced pressure. The residue was chromatographed over silica gel.

Phenol O-Alkylation

To a solution of the phenol previously prepared (1eq) and the appropriate brominated derivative (2eq) in acetonitrile was added a suspension of potassium carbonate (3eq). The reaction mixture was stirred at reflux for 12 hours. The mixture was cooled to room temperature, acidified by a hydrochloric acid 1N solution, and then extracted with ethyl acetate. The combined organic layers were dried over magnesium sulfate, and evaporated under reduced pressure. The residue was chromatographed over silica gel.

Methyl Bromination

A solution of N-bromosuccinimide (1.2eq), benzoyle peroxide (0.08eq), and the phenyltolylmethanone derivative previously prepared (1eq) in chloroform was stirred at reflux under a light source (500 W). The mixture turns brown after 15 minutes of stirring at reflux and the color gradually fades. The mixture was cooled to room temperature and washed with water. The aqueous layer was extracted with dichloromethane. The combined organic layers were dried over magnesium sulfate, and evaporated under reduced pressure. The residue was chromatographed over silica gel.

### Example 7.1.

### (2-((1-ethoxycarbonyl-1,1-dimethylmethyl)oxy)phenyl)(4-(bromomethyl)phenyl)methanone

### 7.1.1

### (2-methoxyphenyl)(p-tolyl)methanone

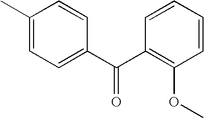

Prepared following the Friedel-Crafts reaction previously described using toluene and 2-methoxybenzoyl chloride. The product was chromatographed over silica gel (eluent cyclohexane/ethyl acetate 95/5). The product was obtained as a colorless oil.

Yield: 28%

Rf (cyclohexane/dichloromethane 9/1): 0.32

IR: νCO: 1661 cm⁻¹

NMR ¹H (CDCl₃): 2.50 (s, 3H); 3.75 (s, 3H); 6.97-7.09 (m, 2H); 7.22-7.26 (d, 2H, J=8.2 Hz); 7.32-7.38 (dd, 1H, J=7.6 Hz, J=1.4 Hz);); 7.42-7.52 (td, 1H, J=8.5 Hz, J=1.4 Hz); 7.71-7.78 (d, 2H, J=7.9 Hz).

### 7.1.2

### (2-hydroxyphenyl)(p-tolyl)methanone

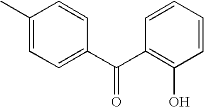

Prepared following the demethylation method previously described using (2-methoxyphenyl)(p-tolyl)methanone (example 7.1.1). The product was chromatographed over silica gel (eluent cyclohexane/ethyl acetate 95/5). The product was obtained as a colorless oil.

This compound was also produced as a by-product of the Friedel-Crafts reaction previously described (example 6.1.1).

Yield: 30%

Rf (cyclohexane/dichloromethane 9/1): 0.32

IR: νCO: 1627 cm⁻¹

NMR ¹H (CDCl₃): 2.52 (s, 3H); 6.85-6.93 (t, 2H, J=7.9 Hz); 7.05-7.11 (d, 1H, J=8.5 Hz); 7.22-7.35 (d, 2H, J=7.9 Hz); 7.45-7.52 (t, 1H, J=8.2Hz); 7.55-7.69 (d, 2H, J=7.9 Hz); 12.09 (s, 1H).

### 7.1.3

### (2-((1-ethoxycarbonyl-1,1-dimethylmethyl)oxy)phenyl)(p-tolyl)methanone

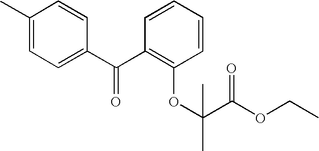

Prepared following the O-alkylation method previously described using (2-hydroxyphenyl)(p-tolyl)methanone (example 7.1.2) and ethyl 2-bromoisobutyrate. The product was chromatographed over silica gel (eluent cyclohexane/ethyl acetate 95/5). The product was obtained as a white powder.

Yield: 40%

Rf (cyclohexane/ethyl acetate 8/2): 0.35

MP: 40-45° C. IR: νCO: 1732 c⁻; 1659 cm⁻¹

NMR ¹H (CDCl₃): 1.24 (t, 3H, J=7.3 Hz); 1.36 (s, 6H); 2.42 (s, 3H); 4.21 (q, 2H, J=7 Hz); 6.77 (d, 1H, J=8.5 Hz); 7.07 (t, 1H, J=7.6 Hz); 7.23 (d, 2H, J=7.9 Hz); 7.33-7.43 (m, 2H); 7.73 (d, 2H, J=8.2 Hz).

### 7.1.4

### 2-((1-ethoxycarbonyl-1,1-dimethylmethyl)oxy)phenyl)-(4-(bromomethyl)phenyl)methanone

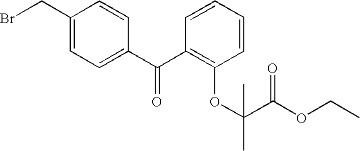

Prepared following the bromination method previously described using (2-((1-ethoxycarbonyl-1,1-dimethylmethyl)oxy)phenyl)(p-tolyl)methanone (example 7.1.3). The product was chromatographed over silica gel (eluent cyclohexane/ethyl acetate 9/1). The product was obtained as a colorless oil.

Yield: 60%

Rf (cyclohexane/ethyl acetate 8/2): 0.70

IR: νCO: 1734 cm⁻¹; 1663 cm⁻¹

NMR ¹H (CDCl₃): 1.24 (t, 3H, J=7 Hz); 1.33 (s, 6H); 4.21 (q, 2H, J=7.3 Hz); 4.52 (s, 2H); 6.75 (d, 1H, J=8.5 Hz); 7.09 (t, 1H, J=7 Hz); 7.39 (t, 1H, J=7.3 Hz); 7.44-7.48 (m, 3H); 7.80 (d, 2H, J=8.2 Hz).

### Example 7.2.

### (3-((1-ethoxycarbonyl-1,1-dimethylmethyl)oxy)phenyl)(4-(bromomethyl)phenyl)methanone

### 7.2.1

### (3-methoxyphenyl)(p-tolyl)methanone

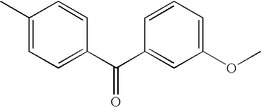

Prepared following the Friedel-Crafts reaction previously described using toluene and 3-methoxybenzoyl chloride. The product was chromatographed over silica gel (eluent cyclohexane/ethyl acetate 9/1). The product was obtained as a colorless oil.

Yield: 72%

Rf (cyclohexane/ethyl acetate 9/1): 0.32

IR: νCO: 1657 cm⁻¹

NMR ¹H (CDCl₃): 2.45 (s, 3H); 3.86 (s, 3H); 7.13 (dd, 1H, J=7.6 Hz, J=1.8 Hz); 7.27-7.30 (d, 2H, J=7.3 Hz); 7.32-7.41 (m, 3H); 7.75 (d, 2H, J=8.2 Hz).

### 7.2.2

### (3-hydroxyphenyl)(p-tolyl)methanone

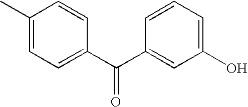

Prepared following the demethylation method previously described using (3-methoxyphenyl)(p-tolyl)methanone (example 7.2.1). The product was chromatographed over silica gel (eluent cyclohexane/ethyl acetate 8/2). The product was obtained as an orange powder.

Yield: 60%

Rf (cyclohexane/ethyl acetate 9/1): 0.20

MP: 113-115° C. IR: νCO: 1638 cm⁻¹

NMR ¹H (CDCl₃): 2.42 (s, 3H); 7.12 (m, 1H); 7.22-7.31 (m, 4H); 7.40 (m, 1H); 7.61 (s, 1H); 7.72 (d, 2H, J=8.2 Hz).

### 7.2.3

### (3-((1-ethoxycarbonyl-1,1-dimethylmethyl)oxy)phenyl)(p-tolyl)methanone

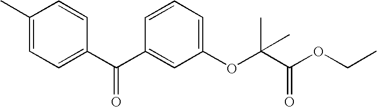

Prepared following the O-alkylation method previously described using (3-hydroxyphenyl)(p-tolyl)methanone (example 7.2.2) and ethyl 2-bromoisobutyrate. The product was chromatographed over silica gel (eluent cyclohexane/ethyl acetate 9/1). The product was obtained as a yellowish oil.

Yield: 87%

Rf (cyclohexane/ethyl acetate 8/2): 0.35

IR: νCO: 1737 cm⁻¹; 1657 cm⁻¹

NMR ¹H (CDCl₃): 1.24 (t, 3H, J=6.9 Hz); 1.62 (s, 6H); 2.43 (s, 3H); 4.22 (q, 2H, J=7.3 Hz); 7.07 (d, 1H, J=7 Hz); 7.25-7.42 (m, 5H); 7.70 (d, 2H, J=8.2 Hz).

### 7.2.4

### (3-((1-ethoxycarbonyl-1,1-dimethylmethyl)oxy)phenyl)(4-(bromomethyl)phenyl)methanone

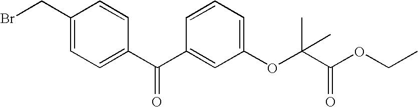

Prepared following the bromination method previously described using (3-((1-ethoxycarbonyl-1,1-dimethylmethyl)oxy)phenyl)(p-tolyl)methanone (example 7.2.3). The product was chromatographed over silica gel (eluent cyclohexane/ethyl acetate 9/1). The product was obtained as a colorless oil.

Yield: 16%

Rf (cyclohexane/ethyl acetate 8/2): 0.30

IR: νCO: 1735 cm⁻¹; 1660 cm⁻¹

NMR ¹H (CDCl₃): 1.24 (t, 3H, J=7 Hz); 1.63 (s, 6H); 4.22 (q, 2H, J=7 Hz); 4.54 (s, 2H); 7.07-7.11 (ddd, 1H, J=7.9 Hz, J=2.6 Hz, J=1.2 Hz); 7.26 (d, 1H, J=1.5 Hz); 7.36 (t, 1H, J=7.9 Hz); 7.44 (dd, 1H, J=7.9 Hz, J=1.2 Hz); 7.51 (d, 2H, J=8.2 Hz); 7.77 (d, 2H, J=8.2 Hz).

### Example 7.3.

### (4-((1-ethoxycarbonyl-1,1-dimethylmethyl)oxy)phenyl)(4-(bromomethyl)phenyl)methanone

### 7.3.1 (4-methoxyphenyl)(p-tolyl)methanone

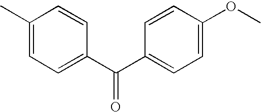

Prepared following the Friedel-Crafts reaction previously described using toluene and 4-methoxybenzoyl chloride. The product was chromatographed over silica gel (eluent cyclohexane/ethyl acetate 9/1). The product was obtained as a white solid.

Yield: 75%

Rf (cyclohexane/ethyl acetate 9/1): 0.32

MP: 77-79° C. IR: νCO: 1644 cm⁻¹

NMR ¹H (CDCl₃): 2.46 (s, 3H);); 3.90 (s, 3H); 6.97 (d, 2H, J=9.1 Hz); 7.29 (d, 2H, J=7.6 Hz); 7.70 (d, 2H, J=7.9 Hz); 7.83 (d, 2H, J=8.8 Hz).

### 7.3.2

### (4-hydroxyphenyl)(p-tolyl)methanone

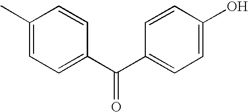

Prepared following the demethylation method previously described using (4-methoxyphenyl)(p-tolyl)methanone (example 7.3.1). The product was chromatographed over silica gel (eluant cyclohexane/ethyl acetate 7/3). The product was obtained as a white powder.

Yield: 86%

Rf (cyclohexane/ethyl acetate 8/2): 0.17

MP: 148-150° C. IR: νCO: 1642 cm⁻¹

NMR ¹H (CDCl₃): 2.46 (s, 3H);); 6.66 (s, 1H); 6.93 (d, 2H, J=8.8 Hz); 7.29 (d, 2H, J=8.8 Hz); 7.70 (d, 2H, J=8.2 Hz); 7.78 (d, 2H, J=8.5 Hz).

### 7.3.3

### (4-((1-ethoxycarbonyl-1,1-dimethylmethyl)oxy)phenyl)(p-tolyl)methanone

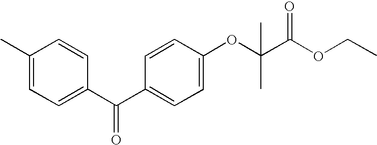

Prepared following the O-alkylation method previously described using (4-hydroxyphenyl)(p-tolyl)methanone (example 7.3.2) and ethyl 2-bromoisobutyrate. The product was chromatographed over silica gel (eluent cyclohexane/ethyl acetate 9/1). The product was obtained as a white solid.

Yield: 79%

Rf (cyclohexane/ethyl acetate 9/1): 0.28

MP: 82-84° C. IR: νCO: 1737 cm⁻¹; 1648 cm⁻¹

NMR ¹H (CDCl₃): 1.25 (t, 3H, J=7.3 Hz); 1.68 (s, 6H); 2.45 (s, 3H); 4.25 (q, 2H, J=7 Hz); 6.87 (d, 2H, J=8.8 Hz); 7.28 (d, 2H, J=7.9 Hz); 7.69 (d, 2H, J=7.9 Hz); 7.75 (d, 2H, J=8.8 Hz).

### 7.3.4

### (4-((1-ethoxycarbonyl-1,1-dimethylmethyl)oxy)-phenyl)(4-(bromomethyl)phenyl)methanone

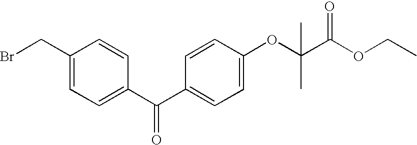

Prepared following the bromination method previously described using (4-((1-ethoxycarbonyl-1,1-dimethylmethyl)oxy)phenyl)(p-tolyl)methanone (example 7.3.3). The product was chromatographed over silica gel (eluent cyclohexane/ethyl acetate 9/1). The product was obtained as a colorless oil.

Yield: 63%

Rf (cyclohexane/ethyl acetate 9/1): 0.25

IR: νCO: 1734 cm⁻¹; 1653 cm⁻¹

NMR ¹H (CDCl₃): 1.19 (t, 3H, J=7 Hz); 1.38 (s, 6H); 4.20 (q, 2H, J=7 Hz); 4.48 (s, 2H); 6.83 (d, 2H, J=8.8 Hz); 7.45 (d, 2H, J=8.2 Hz); 7.61-7.70 (m, 4H).

### Example 8.

### General Procedure for the Preparation of (phenylmethyl)benzyl Bromides

(Phenylmethyl)benzyl bromides were prepared in one step by the reduction of the corresponding (bromomethyl)(phenyl)methanone.

To a solution of the benzoylbenzyl bromide previously prepared (EXAMPLE 6) (1eq) in trifluoroacetic acid (30eq) was added triethylsilane (2.6eq) drop by drop at room temperature. The reaction mixture was then stirred at 50° C. for 1 hour. The reaction mixture was cooled to room temperature before addition of water. The organic layer was partitioned between water and ethyl acetate. The combined organic layers were dried over sodium sulfate, filtered, and evaporated under reduced pressure. The product was chromatographed over silica gel.

### Example 8.1.

### ethyl 2-[3-[(4-bromomethyl)benzyl]phenyloxy]-2-methylpropanoate

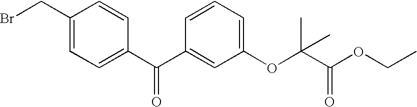

Prepared following the reduction method previously described using (3-((1-ethoxycarbonyl-1,1-dimethylmethyl)oxy)phenyl)-(4-(bromomethyl)phenyl)-methanone (example 7.2.4). The product was chromatographed over silica gel (eluent cyclohexane/ethyl acetate 95/5, then 9/1). The product was obtained as a yellow oil.

Yield: 84%

Rf (cyclohexane/ethyl acetate 8/2): 0.35

NMR ¹H (CDCl₃): 1.19 (t, 3H, J=7 Hz); 1.58 (s, 6H); 3.90 (s, 2H); 4.13 (q, 2H, J=7 Hz); 4.48 (s, 2H); 6.62 (m, 2H); 6.80 (d, 1H, J=7.9 Hz); 7.01-7.21 (m, 3H); 7.30 (d, 2H, J=8.2 Hz).

### Example 8.2.

### ethyl 2-[2-[(4-bromomethyl)benzyl]phenyloxy]-2-methylpropanoate

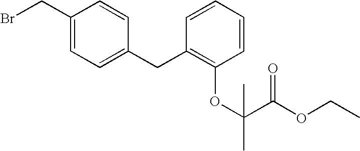

Prepared following the reduction method previously described using (2-((1-ethoxycarbonyl-1,1-dimethylmethyl)oxy)phenyl)-(4-(bromomethyl)phenyl)-methanone (example 7.1.4). The product was chromatographed over silica gel (elution gradient cyclohexane/ethyl acetate 10/0 to 9/1). The product was obtained as a yellow oil.

Yield: 49%

Rf (cyclohexane/ethyl acetate 8/2): 0.34

NMR ¹H (CDCl₃): 1.22 (t, 3H, J=7 Hz); 1.49 (s, 6H); 3.98 (s, 2H); 4.22 (q, 2H, J=7 Hz); 4.50 (s, 2H); 6.62 (d, 1H, J=8 Hz); 6.90 (d, 1H, J=7.9 Hz); 7.00-7.31 (m, 6H).

### Example 8.3.

### ethyl 2-[4-[(4-bromomethyl)benzyl]phenyloxy]-2-methylpropanoate

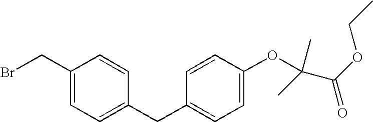

Prepared following the reduction method previously described using (4-((1-ethoxycarbonyl-1,1-dimethylmethyl)oxy)-phenyl)-(4-(bromomethyl)phenyl)-methanone (example 7.3.4). The product was chromatographed over silica gel (eluent cyclohexane/ethyl acetate 95/5). The product was obtained as a yellow oil.

Yield: 49%

Rf (cyclohexane/ethyl acetate 8/2): 0.35

NMR ¹H (CDCl₃): 1.21 (t, 3H, J=7 Hz); 1.58 (s, 6H); 3.90 (s, 2H); 4.21 (q, 2H, J=7 Hz); 4.50 (s, 2H); 6.78 (d, 2H, J=8 Hz); 6.90-7.40 (m, 6H).

### Example 9.

### General Procedure for the Preparation of Phenyloxybenzyl Bromides and Phenylthiobenzyl Bromides

Method 9A: phenyloxybenzyl bromides were prepared in 4 steps using the appropriate methylphenol and the suitable iodoanisole. The phenol function was demethylated, then alkylated. The O-alkylation was followed by a free-radical bromination of the aromatic methyl.

Etherification

Under inert atmosphere, to a solution of the appropriate iodoanisole (1eq) in dioxane were successively added the appropriate methylphenol (1.4eq), copper iodide (I) (0.11eq), N,N-dimethylglycine hydrochloride (0.32eq), and cesium carbonate (2.1eq). The reaction mixture was stirred for 24 hours at 110° C. After cooling down, the mixture was partitioned between water and ethyl acetate. The organic layer was washed with a sodium hydroxide solution (2N) then with brine, then dried over sodium sulfate, filtered, and evaporated under reduced pressure. The residue was chromatographed over silica gel.

Demethylation

Under inert atmosphere, the methoxyether derivative previously prepared was dissolved in dichloromethane. The reaction mixture was cooled to 0° C. before adding a molar solution of boron tribromide in dichloromethane (2eq) drop by drop. The reaction mixture was stirred at 0° C. for 30 minutes, then at room temperature for 3 hours. The mixture was then partitioned between water and dichloromethane. The organic layer was washed with a sodium hydroxide 2N solution. The aqueous layer was acidified to pH 1 and extracted with dichloromethane. The organic layers were combined, dried over sodium sulfate, and evaporated under reduced pressure.

Phenol O-Alkylation

To a solution of the phenol previously prepared (1eq) in dimethylformamide was added potassium (4eq). The suspension was stirred at 80° C. and the brominated derivative (4eq) was added drop by drop. The reaction mixture was stirred at 80° C. for 20 hours. The potassium carbonate was filtered and the dimethylformamide was evaporated under reduced pressure. The residue was partitioned between ethyl acetate and brine. The organic layer was dried over sodium sulfate, filtered, and evaporated under reduced pressure. The product was chromatographed over silica gel.

Methyl Bromination

To a solution of the alkylated product previously prepared (1eq) in carbon tetrachloride were added N-bromosuccinimide (0.95eq) and 2.2′-azo-bis-isobutyronitrile (0.5eq). The reaction mixture was stirred at 80° C. for 1 hour. After cooling down, the reaction mixture was filtered and evaporated under reduced pressure. The residue was taken up in dichloromethane, and washed with aqueous sodium thiosulfate solution then with brine. The organic layer was dried over sodium sulfate, filtered, and evaporated under reduced pressure. The product was chromatographed over silica gel.

Method 9B: phenylthiobenzyl bromides were prepared in 4 steps using the appropriate methylthiophenol and the suitable iodoanisole. The phenol function was demethylated, then alkylated. O-alkylation was followed by free-radical bromination of the aromatic methyl.

Thioetherification

Under inert atmosphere, to a solution of the appropriate iodoanisole (1eq) in dioxane were successively added the appropriate methylthiophenol (1.4eq), copper iodide (I) (0.11eq), N,N-dimethylglycine hydrochloride (0.32eq), and cesium carbonate (2.1eq) The reaction mixture was stirred for 48 hours at 110° C. After cooling down, the mixture was partitioned between water and ethyl acetate. The organic layer was washed with a sodium hydroxide solution (2N), then brine, then dried over sodium sulfate, filtered, and evaporated under reduced pressure. The residue was chromatographed over silica gel.

Demethylation

Under inert atmosphere, the methoxythioether derivative previously prepared (1eq) was dissolved in dichloromethane. The reaction mixture was cooled to 0° C. before adding a molar solution of boron tribromide in dichlorormethane (2eq) drop by drop. The reaction mixture was stirred at 0° C. for 30 minutes, then at room temperature for 6 hours. The mixture was then partitioned between water and dichloromethane. The organic layer was washed with a sodium hydroxide 2N solution. The aqueous layer was acidified to pH 1 and extracted with dichloromethane. The organic layers were combined, dried over sodium sulfate, and evaporated under reduced pressure.

Phenol O-Alkylation

To a solution of the phenol previously prepared (1eq) in dimethylformamide was added potassium carbonate (4eq). The suspension was stirred at 80° C. and the brominated derivative (4eq) was added drop by drop. The reaction mixture was stirred at 80° C. for 20 hours. The potassium carbonate was filtered and the dimethylformamide was evaporated under reduced pressure. The residue was partitioned between ethyl acetate and brine. The organic layer was dried over sodium sulfate, filtered, and evaporated under reduced pressure. The product was chromatographed over silica gel.

Methyl Bromination

To a solution of the alkylated product previously prepared (1eq) in carbon tetrachloride were added N-bromosuccinimide (0.95eq) and 2.2′-azo-bis-isobutyronitrile (0.5eq). The reaction mixture was stirred at 80° C. for 1 hour. After cooling down, the reaction mixture was filtered and evaporated under reduced pressure. The residue was taken up in dichloromethane, and washed with a sodium thiosulfate aqueous solution then with brine. The organic layer was dried over sodium sulfate, filtered, and evaporated under reduced pressure. The product was chromatographed over silica gel.

### Example 9.1.

### tert-butyl 2-[4-((4-bromomethylphenyl)oxy)phenyloxy]-2-methylpropanoate

### 9.1.1

### 1-methoxy-4-(p-tolyoxy)benzene

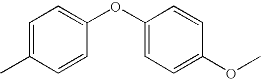

Prepared following the etherification procedure previously described (Method 9A) using 4-methylphenol and 4-iodoanisole. The product was chromatographed over silica gel (eluent cyclohexane/ethyl acetate 95/5). The product was obtained as a white solid.

Yield: 85%

Rf (cyclohexane/ethyl acetate 9/1): 0.55

NMR ¹H (CDCl₃): 2.33 (s, 3H); 3.80 (s, 3H); 6.79-67.02 (m, 6H); 7.10 (d, 2H, J=8.2 Hz).

### 9.1.2

### 4-(p-tolyloxy)phenol

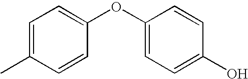

Prepared following the demethylation method previously described (Method 9A) using 1-methoxy-4-(p-tolyloxy)benzene (example 9.1.1). The product was obtained as a beige solid.

Yield: 95%

Rf (cyclohexane/ethyl acetate 8/2): 0.32

NMR ¹H (CDCl₃): 2.30 (s, 3H); 4.86 (s, 1H); 6.72-6.95 (m, 6H); 7.10 (d, 2H, J=8.2 Hz).

### 9.1.3

### tert-butyl 2-methyl-2-((4-(p-tolyloxy)phenyl)oxy)propanoate

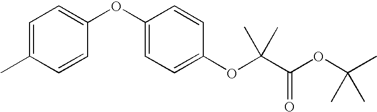

Prepared following the alkylation reaction previously described (Method 9A) using 4-(p-tolyloxy)phenol (example 9.1.2) and tert-butyl 2-bromoisobutyrate. The product was chromatographed over silica gel (eluent cyclohexane/ethyl acetate 9/1). The product was obtained as a yellowish oil.

Yield: 71%

Rf (cyclohexane/ethyl acetate 8/2): 0.65

NMR ¹H (CDCl₃): 1.46 (s, 9H); 1.55 (s, 6H); 2.32 (s, 3H); 6.81-6.95 (m, 6H); 7.11 (d, 2H, J=8.2 Hz).

### 9.1.4

### tert-butyl 2-[4-((4-bromomethyl)phenyloxy)phenyloxy]-2-methylpropanoate

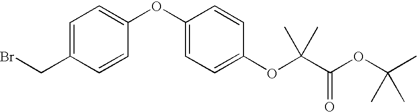

Prepared following the bromination method previously described (Method 9A) using tert-butyl 2-methyl-2-(4-(p-tolyloxy)phenyloxy)propanoate (example 9.1.3). The product was chromatographed over silica gel (eluent cyclohexane/ethyl acetate 95/5). The product was obtained as a colorless oil.

Yield: 67%

Rf (cyclohexane/ethyl acetate 9/1): 0.45

NMR ¹H (CDCl₃): 1.47 (s, 9H); 1.58 (s, 6H); 4.50 (s, 2H); 6.81-6.98 (m, 6H); 7.32 (d, 2H, J=8.2 Hz).

### Example 9.2.

### tert-butyl 2-[3-((4-bromomethyl)phenyloxy)phenyloxy]-2-methylpropanoate

### 9.2.1

### 1-methoxy-3-(p-tolyloxy)benzene

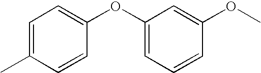

Prepared following the etherification procedure previously described (Method 9A) using 4-methylphenol and 3-iodoanisole. The product was chromatographed over silica gel (eluent cyclohexane/ethyl acetate 9/1). The product was obtained as a yellow oil.

Yield: 93%

Rf (cyclohexane/ethyl acetate 9/1): 0.55

NMR ¹H (CDCl₃): 2.33 (s, 3H); 3.76 (s, 3H); 6.42-6.70 (m, 3H); 6.92 (d, 2H, J=8.2 Hz); 7.08-7.25 (m, 3H).

### 9.2.2

### 3-(p-tolyloxy)phenol

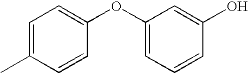

Prepared following the demethylation method previously described (Method 9A) using 1-methoxy-3-(p-tolyloxy)benzene (example 9.2.1). The product was obtained as a yellow oil.

Yield: 98%

Rf (cyclohexane/ethyl acetate 8/2): 0.32

NMR ¹H (CDCl₃): 2.32 (s, 3H); 5.19 (s, 1H); 6.45 (s, 1H); 6.55 (d, 2H, J=8 Hz); 6.92 (d, 2H, J=8 Hz); 7.05-7.20 (m, 3H).

### 9.2.3

### tert-butyl 2-methyl-2-(3-(p-tolyloxy)phenyloxy)propanoate

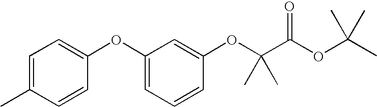

Prepared following the alkylation reaction previously described (Method 9A) using 3-(p-tolyloxy)phenol (example 9.2.2) and tert-butyl 2-bromoisobutyrate. The product was chromatographed over silica gel (eluent cyclohexane/ethyl acetate 95/5). The product was obtained as a yellow oil.

Yield: 63%

Rf (cyclohexane/ethyl acetate 8/2): 0.65

NMR ¹H (CDCl₃): 1.41 (s, 9H); 1.55 (s, 6H); 2.32 (s, 3H); 6.45-6.65 (m, 3H); 6.90 (d, 2H, J=8.2 Hz); 7.08-7.20 (m, 3H).

### 9.2.4

### tert-butyl 2-[3-((4-bromomethyl)phenyloxy)phenyloxy]-2-methylpropanoate

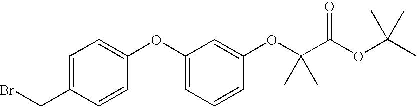

Prepared following the bromination method previously described (Method 9A) using tert-butyl 2-methyl-2-(3-(p-tolyloxy)phenyloxy)propanoate (example 9.2.3). The product was chromatographed over silica gel (eluent cyclohexane/ethyl acetate 95/5). The product was obtained as an orange oil.

Yield: 66%

Rf (cyclohexane/ethyl acetate 9/1): 0.45

NMR ¹H (CDCl₃): 1.42 (s, 9H); 1.57 (s, 6H); 4.50 (s, 2H); 6.45-6.70 (m, 3H); 6.95 (d, 2H, J=8 Hz); 7.05-7.25 (m, 1H); 7.35 (d, 2H, J=8 Hz).

### Example 9.3.

### tert-butyl 2-[4-((4-bromomethyl)phenylthio)phenyloxy]-2-methylpropanoate

### 9.3.1

### 1-methoxy-4-(p-tolylthio)benzene

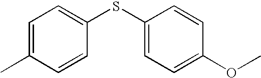

Prepared following the thioetherification method previously described (Method 9B) using 4-methylbenezenethiol and 4-iodoanisole. The product was chromatographed over silica gel (eluent cyclohexane/ethyl acetate 95/5). The product was obtained as a white solid.

Yield: 75%

Rf (cyclohexane/ethyl acetate 9/1): 0.45

NMR ¹H (CDCl₃): 2.28 (s, 3H); 3.82 (s, 3H); 6.85 (d, 2H, J=8.2 Hz); 7.04 (d, 2H, J=8.2 Hz); 7.12 (d, 2H, J=8.2 Hz); 7.37 (d, 2H, J=8.2 Hz).

### 9.3.2

### 4-(p-tolylthio)phenol

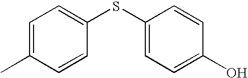

Prepared following the demethylation method previously described (Method 9B) using 1-methoxy-4-(p-tolylthio)benzene (example 9.2.1). The product was obtained as a beige solid.

Yield: 69%

Rf (cyclohexane/ethyl acetate 8/2): 0.34

NMR ¹H (CDCl₃): 2.30 (s, 3H); 5.64 (s, 1H); 6.81 (d, 2H, J=8.2 Hz); 7.01-7.21 (m, 4H); 7.30 (d, 2H, J=8.2 Hz)

### 9.3.3

### tert-butyl 2-methyl-2-(4-p-tolylthio)phenyloxy)propanoate

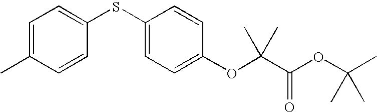

Prepared following the alkylation reaction previously described (Method 9B) using 4-(p-tolylthio)phenol (example 9.1.2) and tert-butyl 2-bromoisobutyrate. The product was chromatographed over silica gel (eluent cyclohexane/ethyl acetate 98/2). The product was obtained as a yellowish oil.

Yield: 86%

Rf (cyclohexane/ethyl acetate 95/5): 0.65

NMR ¹H (CDCl₃): 1.42 (s, 9H); 1.59 (s, 6H); 2.30 (s, 3H); 6.80 (d, 2H, J=8.2 Hz); 7.08 (d, 2H, J=8 Hz); 7.15 (d, 2H, J=8 Hz); 7.28 (d, 2H, J=8.2 Hz).

### 9.3.4

### tert-butyl 2-[4-((4-bromomethyl)phenylthio)phenyloxy]-2-methylpropanoate

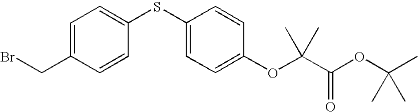

Prepared according to the bromination method previously described (Method 9B) using tert-butyl 2-methyl-2-(4-p-(tolylthio)phenyloxy)propanoate (example 9.3.3). The product was chromatographed over silica gel (eluent cyclohexane/ethyl acetate 95/5). The product was obtained as an orange oil.

Yield: 36%

Rf (cyclohexane/ethyl acetate 9/1): 0.45

NMR ¹H (CDCl₃): 1.45 (s, 9H); 1.60 (s, 6H); 4.45 (s, 2H); 6.83 (d, 2H, J=8.2 Hz); 7.10 (d, 2H, J=8 Hz); 7.25 (d, 2H, J=8 Hz); 7.35 (d, 2H, J=8.2 Hz).

### Example 9.4.

### tert-butyl 2-[3-((4-bromomethyl)phenylthio)phenyloxy]-2-methylpropanoate

### 9.4.1

### 1-methoxy-3-(p-tolylthio)benzene

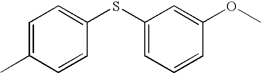

Prepared following the thioetherification method previously described (Method 9B) using 4-methylbenezenethiol and 3-iodoanisole. The product was chromatographed over silica gel (eluent cyclohexane/ethyl acetate 95/5). The product was obtained as a yellow oil.

Yield: 33%

Rf (cyclohexane/ethyl acetate 9/1): 0.45

NMR ¹H (CDCl₃): 2.36 (s, 3H); 3.72 (s, 3H); 6.65-6.95 (m, 3H); 7.02-7.25 (m, 3H); 7.30 (d, 2H, J=8 Hz).

### 9.4.2

### 3-(p-tolylthio)phenol

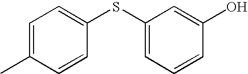

Prepared following the demethylation method previously described (Method 9B) using 1-methoxy-3-(p-tolylthio)benzene (example 9.4.1). The product was obtained as a yellow oil.

Yield: 72%

Rf (cyclohexane/ethyl acetate 8/2): 0.34

NMR ¹H (CDCl₃): 2.35 (s, 3H); 4.90 (s, 1H); 6.61 (d, 1H, J=8Hz); 6.65 (s, 1H); 6.81 (dd, 1H, J=8 Hz, J=2 Hz); 7.02-7.23 (m, 3H); 7.35 (d, 2H, J=8 Hz).

### 9.4.3

### tert-butyl 2-methyl-2-(3-(p-tolyloxy)phenylthio)propanoate

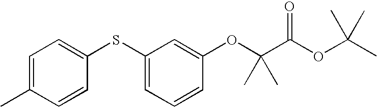

Prepared following the alkylation reaction previously described (Method 9B) using 3-(p-tolyloxy)phenol (example 9.4.2) and tert-butyl 2-bromoisobutyrate. The product was chromatographed over silica gel (eluent cyclohexane/ethyl acetate 98/2). The product was obtained as a yellow oil.

Yield: 74%

Rf (cyclohexane/ethyl acetate 8/2): 0.68

NMR ¹H (CDCl₃): 1.39 (s, 9H); 1.51 (s, 6H); 2.35 (s, 3H); 6.68 (dd, 1H, J=8 Hz, J=2 Hz); 6.76 (t, 1H, J=2 Hz); 6.83 (dd, 1H, J=8 Hz, J=2 Hz); 7.05-7.19 (m, 3H); 7.29 (d, 2H, J=8 Hz).

### 9.4.4

### tert-butyl 2-[3-((4-bromomethyl)phenylthio)phenyloxy]-2-methylpropanoate

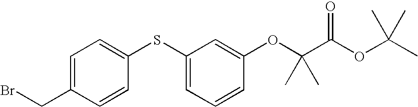

Prepared according to the bromination method previously described (Method 9B) using tert-butyl 2-methyl-2-(3-(p-tolylthio)phenyloxy)propanoate (example 9.4.3). The product was chromatographed over silica gel (eluent cyclohexane/ethyl acetate 95/5). The product was obtained as a yellow oil.

Yield: 64%

Rf (cyclohexane/ethyl acetate 9/1): 0.48

NMR ¹H (CDCl₃): 1.40 (s, 9H); 1.53 (s, 6H); 4.45 (s, 2H); 6.68 (dd, 1H, J=8 Hz, J=2 Hz); 6.76 (t, 1H, J=2 Hz); 6.83 (dd, 1H, J=8 Hz, J=2 Hz); 7.05-7.19 (m, (d, 2H, J=8 Hz).

### Example 10.

### General Procedure for the Preparation of Bromobenzenes

Method 10A: the acylation of the appropriate bromobenzene was followed by a reduction of the carbonyl function

Friedel-Craft's Reaction

Under inert atmosphere, to a solution of aluminium trichloride (1.25eq) in dichloromethane cooled at 0° C. was added the appropriate bromobenzene (1eq) drop by drop during 10 minutes. The reaction mixture was stirred at 0° C. for 1 hour then was added acyl chloride (1.05eq) in dichloromethane drop by drop. The reaction mixture was stirred for 2 hours then poured into ice. The phases were separated. The organic layer was washed with brine, dried over magnesium sulfate and evaporated under reduced pressure. The residue was chromatographed over silica gel.

Reduction of the Carbonyl Function

Under inert atmosphere, to a solution of the ketone previously prepared (1eq) in dichloromethane cooled at 0° C. were added boron trifluoride diethyletherate (2eq) then triethylsilane (3eq) drop by drop. The recation mixture was stirred at room temperature for 24 hours then water was added. The organic layer was washed with brine, dried over magnesium sulfate and evaporated under reduced pressure. The residue was chromatographed over silica gel.

Method 10B: the bromophenol was prepared using the appropriate fluorobromobenzene with methanesulfonylethanol.

Under inert atmosphere, to a solution of the fluorinated derivative in dimethylformamide was added methylsulfonylethanol (1.5eq). The mixture was stirred at 0° C. before adding sodium hydride (5eq). The reaction mixture was stirred at room temperature, then acidified with 1M hydrochloric acid solution to pH 2, and then extracted with ethyl acetate. The organic layer was washed with brine, dried over magnesium sulfate and evaporated under reduced pressure. The residue was chromatographed over silica gel.

Method 10C: bromination of the appropriate benzyl alcohol.

To a solution of the benzyl alcohol (1eq) in toluene at 0° C. was added boron tribromide (1eq). The reaction mixture was stirred at room temperature for 12 hours. The reaction was poured into ice, then extracted with toluene. The organic layers were dried over magnesium sulphate, filtered and evaporated under reduced pressure. The brominated derivative was used without any further purification.

Method 10D: using the appropriate methyl benzoate.

Free-Radical Bromination of Methylbenzene

A solution of methylbenzene (1eq), N_bromosuccinimide (1.1eq), and benzoyle peroxide (0.01eq) in dichloromethane was stirred at reflux under irradiation of a 75W lamp for 24 hours. The reaction mixture was washed with water, then with brine. The organic layer was dried over magnesium sulfate, filtered and evaporated under reduced pressure. The product was purified by recristallization.

Alkylation with an Organomagnesium Derivative

The bromomethylbenzene previously prepared (1eq) and copper iodide (0.1eq) were dissolved in tetrahydrofuran under argon atmosphere. The solution was stirred for 15 min at −40° C. (dry ice/acetonitrile bath), then methylmagnesium bromide (1.1eq) was added. The reaction mixture was slowly warmed to 0° C. and stirred for 2 hours before adding an ammonium chloride 2.5M solution. The mixture was extracted with dichloromethane. The organic layer was washed with brine, dried over magnesium sulfate and evaporated under reduced pressure. The residue was chromatographed over silica gel.

Ester Reduction

The ester previously prepared (1eq) was dissolved in anhydrous THF. The reaction mixture was cooled in a bath of ice with sodium chloride, then lithium tetrahydroaluminate (1eq) was added in portions. The reaction mixture was slowly warmed to room temperature and stirred for 12 hours at room temperature. Then water, then sodium hydroxide 2N solution, then water were added and the mixture was stirred for 15 minutes. The precipitate was filtered off. The filtrate was evaporated and the residue was used without any further purification. Alcohol bromination. According to the method previously described (Method 10C)

Method 10E: using the appropriate benzyl alcohol.

Aromatic Bromination of the Benzyl Alcohol

The benzyl alcohol was dissolved in an equivolumetric acetonitrile/water mixture. Potassium bromide, then sodium hydrogenosulfite were added and the reaction mixture was stirred at room temperature for 1 h30. A sodium bisulfate 10% solution was a added and the mixture was extracted with diethyl ether. The organic layer was washed with a saturated sodium carbonate solution, dried over magnesium sulfate, filtered and evaporated under reduced pressure. The product was chromatographed over silica gel.

Alcohol Bromination

According to the method previously described (Method 10C).

Method 10F: using the appropriate benzylic acid.

### Acid Reduction

To a solution of the acid (1eq) in anhydrous tetrahydrofuran under argon atmosphere were added boron dimethylsulfite 2M solution in tetrahydrofuran drop by drop. The reaction mixture was stirred for 48 hours at room temperature. The tetrahydrofuran was evaporated and the residue was taken up in water and extracted with dichloromethane. The organic layer was washed with brine, dried over magnesium sulfate and evaporated under reduced pressure. The product was used without any further purification.

Alcohol Bromination

According to the method previously described (Method 10C).

### Example 10.1.

### 3-Bromo-4-ethylanisole and 3-Bromo-6-ethylanisole

### 10.1.1

### 1-(2-Bromo-4-methoxyphenyl)ethanone and 1-(4-Bromo-2-methoxyphenyl)ethanone

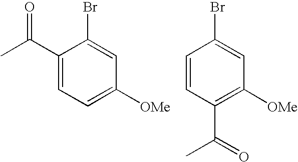

Prepared following the Friede-Craft's method previously described (Method 10A) using 3-bromoanisole and acetyl chloride. The products were chromatographed over silica gel (elution gradient petroleum ether/ethyl acetate 98/2 to 90/10). 1-(2-bromo-4-methoxyphenyl)ethanone was obtained as a colorless oil and 1-(4-bromo-2-methoxyphenyl)ethanone was obtained as a white solid.

Yield: 55% (1-(2-bromo-4-methoxyphenyl)ethanone) and 18% (1-(4-bromo-2-methoxyphenyl)ethanone)

Rf (petroleum ether/ethyl acetate 95/5): 0.33 (1-(2-bromo-4-methoxyphenyl)ethanone) and 0.5 (1-(4-bromo-2-methoxyphenyl)ethanone)

NMR ¹H (CDCl₃) (1-(2-bromo-4-methoxyphenyl)ethanone): 2.60 (s, 3H); 3.82 (s, 3H); 6.85 (d, 1H, J=1.9 Hz); 7.13 (s, 1H); 7.58 (d, 1H, J=5.0 Hz).

NMR ¹H (CDCl₃) (1-(4-bromo-2-methoxyphenyl)ethanone): 2.61 (s, 3H); 3.94 (s, 3H); 7.15 (m, 2H); 7.64 (d, 1H, J=8.3 Hz).

### 10.1.2

### 3-Bromo-4-ethylanisole

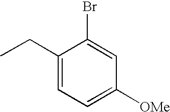

Prepared following the reduction method previously described (Method 10A) using (1-(2-bromo-4-methoxyphenyl)ethanone) (example 10.1.1). The product was chromatographed over silica gel (eluent petroleum ether/ethyl acetate 98/2). The product was obtained as a colorless oil.

Yield: 93%

Rf (petroleum ether/ethyl acetate 95/5): 0.5

NMR ¹H (DMSO): 1.10 (t, 3H, J=7.5 Hz); 2.67 (q, 2H); 3.71 (s, 3H); 6.80 (dd, 1H, J=8.3 Hz and J=2.8 Hz); 6.93 (d, 1H, J=2.8 Hz); 7.05 (d, 1H, J=8.3 Hz).

### 10.1.3

### 3-Bromo-6-ethylanisole

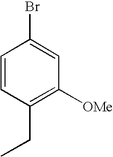

Prepared following the reduction method previously described (Method 10A) using (1-(4-bromo-2-methoxyphenyl)ethanone) (example 10.1.1). The product was chromatographed over silica gel (eluent petroleum ether/ethyl acetate 98/2). The product was obtained as a colorless oil.

Yield: 82%

Rf (petroleum ether/ethyl acetate 95/5): 0.8

NMR ¹H (CDCl₃): 1.14 (t, 3H, J=7.5 Hz); 2.54 (q, 2H); 3.74 (s, 3H); 6.92 (d, 1H, J=1.7 Hz); 6.96-7.02 (m, 2H).

### Example 10.2.

### 3-Bromo-4-propylanisole and 3-Bromo-6-propylanisole

### 10.2.1

### 1-(2-Bromo-4-methoxyphenyl)propan-1-one and 1-(4-Bromo-2-methoxyphenyl)propan-1-one

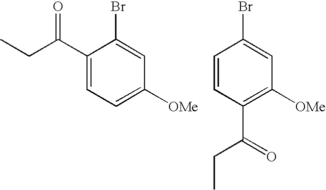

Prepared following the Friedel-Craft's method previously described (Method 10A) using 3-bromoanisole and propionyl chloride. The products were chromatographed over silica gel (elution gradient petroleum ether/ethyl acetate 99/1 to 95/5). 1-(2-bromo-4-methoxyphenyl)propan-1-one was obtained as a pale yellow solid and 1-(4-bromo-2-methoxyphenyl)propan-1-one was obtained as a white solid.

Yield: 33% (1-(2-bromo-4-methoxyphenyl)propan-1-one) and 23% (1-(4-bromo-2-methoxyphenyl)propan-1-one)

Rf (petroleum ether/ethyl acetate 95/5): 0.15 (1-(2-bromo-4-methoxyphenyl)propan-1-one) and 0.33 (1-(4-bromo-2-methoxyphenyl)propan-1-one)

NMR ¹H (CDCl₃) (1-(2-bromo-4-methoxyphenyl)propan-1-one): 1.18 (t, 3H, J=7.3 Hz); 2.91 (q, 2H, J=7.3 Hz); 3.81 (s, 3H); 6.85 (dd, 1H, J=8.6 Hz and J=2.5 Hz); 7.11 (d, 1H, J=2.5 Hz); 7.47 (d, 1H, J=8.6 Hz).

NMR ¹H (CDCl₃) (1-(4-bromo-2-methoxyphenyl)propan-1-one): 1.24 (t, 3H, J=7.2 Hz); 3.00 (q, 2H, J=7.2 Hz); 7.03 (dd, 1H, J=8.6 Hz and J=1.9 Hz); 7.18 (d, 1H, J=1.9 Hz); 7.61 (d, 1H, J=8.6 Hz); 12.44 (s, 1 H).

### 10.2.2

### 3-Bromo-4-propylanisole

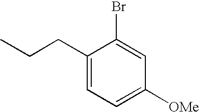

Prepared following the reduction method previously described (Method 10A) using 1-(2-bromo-4-methoxyphenyl)propan-1-one (example 10.2.1). The product was chromatographed over silica gel (eluent petroleum ether/ethyl acetate 98/2). The product was obtained as a colorless oil.

Yield: 77%

Rf (petroleum ether/ethyl acetate 95/5): 0.4

NMR ¹H (CDCl₃): 0.95 (t, 3H, J=7.3 Hz); 1.53-1.68 (m, 2H); 2.60-2.66 (m, 2H); 3.76 (s, 3H); 6.78 (dd, 1H, J=8.6 Hz J=2.6 Hz); 7.07-7.11 (m, 2H).

### 10.2.3

### 3-Bromo-6-propylanisole

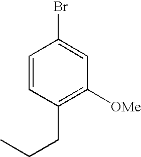

Prepared following the reduction method previously described (Method 10A) using 1-(4-bromo-2-methoxyphenyl)propan-1-one (example 10.2.1). The product was chromatographed over silica gel (eluent petroleum ether/ethyl acetate 98/2). The product was obtained as a colorless oil.

Yield: 82%

Rf (petroleum ether/ethyl acetate 95/5): 0.4

NMR ¹H (CDCl₃): 0.91 (t, 3H, J=7.3 Hz); 1.48-1.62 (m, 2H); 2.48-2.54 (m, 2H); 3.79 (s, 3H); 6.93-6.98 (m, 3H).

### Example 10.3.

### 3-Bromo-4-isobutylanisole and 3-Bromo-6-isobutylanisole

### 10.3.1

### 1-(2-bromo-4-methoxyphenyl)-2-methylpropan-1-one and 1-(4-bromo-2-methoxyphenyl)-2-methylpropan-1-one

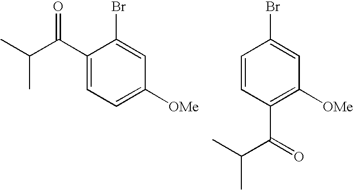

Prepared following the Friedel-Craft's method previously described (Method 10A) using 3-bromoanisole and isobutyl chloride. The products were chromatographed over silica gel (elution gradient petroleum ether/ethyl acetate 99/1 to 98/2). The products were obtained as a pale yellow oil.

Yield: 10% (1-(2-bromo-4-methoxyphenyl)-2-methylpropan-1-one) and 16% (1-(4-bromo-2-methoxyphenyl)-2-methylpropan-1-one)

Rf (petroleum ether/ethyl acetate 90/50): 0.3 (1-(2-bromo-4-methoxyphenyl)-2-methylpropan-1-one) and 0.4 (1-(4-bromo-2-methoxyphenyl)-2-methylpropan-1-one)

NMR ¹H (CDCl₃) (1-(2-bromo-4-methoxyphenyl)-2-methylpropan-1-one): 1.15 (d, 6H, J=6.9 Hz); 3.28-3.45 (m, 1H); 3.80 (s, 3H); 6.85 (dd, 1H, J=8.6 Hz J=2.4 Hz); 7.11 (d, 1H, J=2.4 Hz); 7.33 (d, 1H, J=8.6 Hz).

NMR ¹H (CDCl₃) (1-(4-bromo-2-methoxyphenyl)-2-methylpropan-1-one): 1.12 (d, 6H, J=6.9 Hz); 3.35-3.52 (m, 1H); 3.87 (s, 3H); 7.10-7.14 (m, 2H); 7.40 (d, 1H, J=8 Hz).

### 10.3.2

### 3-bromo-4-isobutylanisole

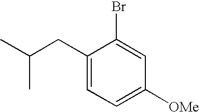

Prepared following the reduction method previously described (Method 10A) using 1-(2-bromo-4-methoxyphenyl)-2-methylpropan-1-one (example 10.3.1). The product was chromatographed over silica gel (eluent petroleum ether/ethyl acetate 98/2). The product was obtained as a colorless oil.

Yield: 82%

Rf (petroleum ether/ethyl acetate 90/50): 0.7

NMR ¹H (CDCl₃): 0.90 (d, 6H, J=6.7 Hz); 1.92 (hept, 1H, J=6.7 Hz); 2.54 (d, 2H, J=7.2 Hz); 3.71 (s, 3H); 6.73 (dd, 1H, J=8.5 Hz J=2.7 Hz); 7.02 (d, 1H, J=8.5 Hz); 7.07 (d, 1H, J=2.7 Hz).

### 10.3.3

### 4-bromo-6-isobutylanisole

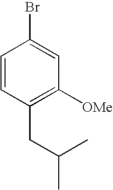

Prepared following the reduction method previously described (Method 10A) using 1-(4-bromo-2-methoxyphenyl)-2-methylpropan-1-one (example 10.3.1). The product was chromatographed over silica gel (eluent petroleum ether/ethyl acetate 98/2). The product was obtained as a colorless oil.

Yield: 89%

Rf (petroleum ether/ethyl acetate 90/50): 0.75

NMR ¹H (CDCl₃): 0.87 (d, 6H, J=6.6 Hz); 1.78-1.94 (m, 1H); 2.41 (d, 2H, J=7.1 Hz); 3.79 (s, 3H); 6.91-7.02 (m, 3H).

### Example 10.4.

### 2-bromo-4-hydroxybenzonitrile

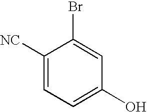

Prepared following the method previously described (Method 10B) using 2-bromo-4-fluorobenzonitrile. The product was chromatographed over silica gel (elution gradient petroleum ether/ethyl acetate 90/10 to 80/20). The product was obtained as a white solid.

Yield: 95%

Rf (petroleum ether/ethyl acetate 70/30): 0.25

NMR ¹H (CDCl₃): 5.95 (s, 1H); 6.87 (dd, 1H, J=8.5 Hz, J=2.4 Hz); 7.17 (d, 1H, J=2.4 Hz); 7.54 (d, 1H, J=8.5 Hz).

### Example 10.5.

### 4-bromo-1-(bromomethyl)-2-methoxybenzene

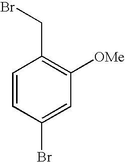

Prepared following the bromination method previously described (Method 10C) using 4-bromo-1-(hydroxymethyl)-2-methoxybenzene. The product was obtained as a white solid.

Yield: 69%

Rf (petroleum ether): 0.75

NMR ¹H (CDCl₃): 3.92 (s, 3H); 4.53 (s, 2H); 7.05 (s, 1H); 7.10 (d, 1H, J=7.5 Hz); 7.22 (d, 1H, J=7.5 Hz).

### Example 10.6.

### 4-bromo-1-(bromomethyl)-2-ethylbenzene

### 10.6.1

### Methyl 4-bromo-3-(bromomethyl)benzoate

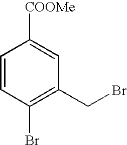

Prepared following the free-radical bromination method previously described (Method 10D) using methyl 4-bromo-3-methylbenzoate. The product was purified by recristallization in heptane. The product was obtained as a white solid.

Yield: 76%

Rf (petroleum ether/ethyl acetate 90/50): 0.45

NMR ¹H (CDCl₃): 3.95 (s, 3H); 4.64 (s, 2H); 7.68 (d, 1H, J=7.5 Hz); 7.83 (d, 1H, J=7.5 Hz); 8.13 (d, 1H, J=2.5 Hz).

### 10.6.2

### Methyl 4-bromo-3-ethylbenzoate

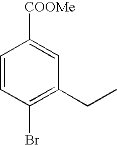

Prepared following the alkylation with an organomagnesium derivative method previously described (Method 10D) using methyl 4-bromo-3-(bromomethyl)benzoate (example 10.6.1). The product was chromatographed over silica gel (elution gradient petroleum ether/ethyl acetate 98/2). The product was obtained as a colorless oil.

Yield: 54%

Rf (petroleum ether/ethyl acetate 90/10): 0.53

NMR ¹H (CDCl₃): 1.28 (t, 3H, J=7.5 Hz); 2.83 (q, 2H, J=7.5 Hz); 3.93 (s, 3H); 7.62 (d, 1H, J=7.5 Hz); 7.72 (dd, 1H, J=7.5 Hz, J=2.5 Hz); 7.92 (d, 1H, J=2.5 Hz).

### 10.6.3

### (4-bromo-3-ethylphenyl)methanol

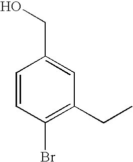

Prepared following the reduction method previously described (Method 10D) using methyl 4-bromo-3-ethylbenzoate (example 10.6.2). The product was obtained as a colorless oil.

Yield: 75%

Rf (petroleum ether/ethyl acetate 90/10): 0.28

NMR ¹H (CDCl₃): 1.26 (t, 3H, J=7.5 Hz); 2.04 (s, 1H); 2.84 (q, 2H, J=7.5 Hz); 4.64 (s, 2H); 7.07 (d, 1H, J=7.5 Hz); 7.25 (s, 1H); 7.53 (d, 1H, J=7.5 Hz).

### 10.6.4

### 4-bromo-1-(bromomethyl)-2-ethylbenzene

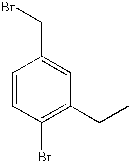

Prepared following the bromination method previously described (Method 10C) using (4-bromo-3-ethylphenyl)methanol (example 10.6.3). The product was obtained as a colorless oil.

Yield: 79%

Rf (petroleum ether): 0.36

NMR ¹H (CDCl₃): 1.28 (t, 3H, J=7.5 Hz); 2.80 (q, 2H, J=7.5 Hz); 4.47 (s, 2H); 7.12 (dd, 1H, J=7.5 Hz, J=2.5 Hz); 7.29 (s, 1H); 7.54 (d, 1H, J=7.5 Hz).

### Example 10.7.

### 1-bromo-4-(bromomethyl)-2-propylbenzene

### 10.7.1

### Methyl 4-bromo-3-propylbenzoate

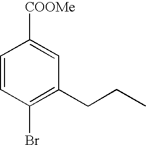

Prepared following the alkylation method with methylmagnesium bromide previously described (Method 10D) using methyl 4-bromo-3-(bromomethyl)benzoate (example 10.6.1). The product was chromatographed over silica gel (eluent petroleum ether/ethyl acetate 98/2). The product was obtained as a yellow oil.

Yield: 33%

Rf (petroleum ether): 0.45

NMR ¹H (CDCl₃): 1.01 (t, 3H, J=7.5 Hz); 1.62-1.77 (m, 2H); 2.77 (t, 2H, J=7.5 Hz); 3.93 (s, 3H); 7.62 (d, 1H, J=7.5 Hz); 7.72 (dd, 1H, J=7.5 Hz, J=2.5 Hz); 7.90 (d, 1H, J=2.5 Hz).

### 10.7.2

### (4-bromo-3-propylphenyl)methanol

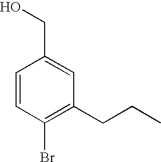

Prepared following the reduction method previously described (Method 10D) using methyl 4-bromo-3-propylbenzoate (example 10.7.1). The product was obtained as a yellow solid.

Yield: 73%

Rf (petroleum ether/ethyl acetate 90/10): 0.73

NMR ¹H (CDCl₃): 1.02 (t, 3H, J=7.5 Hz); 1.60-1.75 (m, 2H); 1.90 (s, 1H); 2.73 (q, 2H, J=7.5 Hz); 4.65 (s, 2H); 7.06 (dd, 1H, J=7.5 Hz, J=2.5 Hz); 7.23 (d, 1H, J=2.5 Hz); 7.53 (d, 1H, J=7.5 Hz).

### 10.7.3

### 4-bromo-1-(bromomethyl)-2-propylbenzene

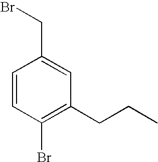

Prepared following the bromination method previously described (Method 10C) using (4-bromo-3-propylphenyl)methanol (example 10.7.2). The product was obtained as a colorless oil.

Yield: 96%

Rf (petroleum ether): 0.5

NMR ¹H (CDCl₃): 1.03 (t, 3H, J=7.5 Hz); 1.61.1.76 (m, 2H); 2.73 (t, 2H, J=7.5 Hz); 4.46 (s, 2H); 7.11 (dd, 1H, J=7.5 Hz J=2.5 Hz); 7.27 (d, 1H); 7.53 (d, 1H, J=7.5 Hz).

### Example 10.8.

### 1-bromo-4-(bromomethyl)-2-methoxybenzene

### 10.8.1

### (4-bromo-3-methoxyphenyl)methanol

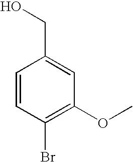

Prepared following the aromatic bromination method previously described (Method 10E) using (3-methoxyphenyl)methanol. The product was chromatographed over silica gel (eluent petroleum ether/ethyl acetate 95/5). The product was obtained as a beige solid.

Yield: 86%

Rf (petroleum ether/ethyl acetate 90/10): 0.57

NMR ¹H (CDCl₃): 3.66 (s, 1H); 3.73 (s, 3H); 4.61 (s, 2H); 6.64 (dd, 1H, J=7.5 Hz, J=2.5 Hz); 7.01 (d, 1H, J=2.5 Hz); 7.34 (d, 1H, J=7.5 Hz).

### 10.8.2

### 1-bromo-4-(bromomethyl)-2-methoxybenzene

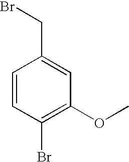

Prepared following the bromination method previously described (Method 10C) using (4-bromo-3-methoxyphenyl)methanol (example 10.8.1). The product was obtained as a colorless oil.

Yield: 88%

Rf (petroleum ether): 0.7

NMR ¹H (CDCl₃): 3.82 (s, 3H); 4.58 (s, 2H); 6.76 (dd, 1H, J=7.5 Hz and J=2.5 Hz); 7.02 (d, 1H, J=2.5 Hz); 7.48 (d, 1H, J=7.5 Hz).

### Example 10.9.

### 1-bromo-4-(bromomethyl)-3-methylbenzene

### 10.9.1

### (4-bromo-2-methylphenyl)methanol

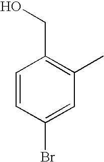

Prepared following the ester reduction method previously described (Method 10F) using 4-bromo-3-methylbenzoic acid. The product was obtained as a colorless oil.

Yield: 75%

Rf (petroleum ether/ethyl acetate 90/10): 0.75

NMR ¹H (CDCl₃): 1.95 (s, 1H); 2.33 (s, 3H); 4.63 (s, 2H); 7.22-7.36 (m, 3H).

### 10.9.2

### 1-bromo-4-(bromomethyl)-3-methylbenzene

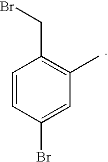

Prepared following the bromination method previously described (Method 10C) using (4-bromo-2-methylphenyl)methanol (example 10.9.1). The product was obtained as a yellow oil.

Yield: 98%

Rf (petroleum ether): 0.5

NMR ¹H (CDCl₃): 2.42 (s, 3H); 4.48 (s, 2H); 7.20 (d, 1H, J=7.5 Hz); 7.33 (d, 1H, J=7.5 Hz, J=2.5 Hz); 7.38 (s, 1H, J=7.5 Hz).

### Example 10.10.

### 1-bromo-4-(bromomethyl)-2-trifluoromethylbenzene

### 10.10.1

### (4-bromo-3-(trifluoromethyl)phenyl)methanol

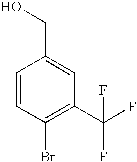

Prepared following the acid reduction method previously described (Method 10F) using 4-bromo-2-trifluoromethylbenzoic acid. The product was obtained as a white solid.

Yield: 93%

Rf (petroleum ether/ethyl acetate 90/10): 0.3

NMR ¹H (CDCl₃): 4.69 (s, 2H); 7.36 (d, 1H, J=7.5 Hz); 7.66-7.68 (m, 2H).

### 10.10.2

### 1-bromo-4-(bromomethyl)-2-trifluoromethylbenzene

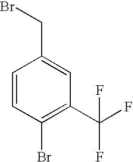

Prepared following the bromination method previously described (Method 10C) using (4-bromo-3-(trifluoromethyl)phenyl)methanol (example 10.10.1). The product was obtained as a white solid.

Yield: 92%

Rf (petroleum ether): 0.57

NMR ¹H (CDCl₃): 4.45 (s, 2H); 7.42 (d, 1H, J=7.5 Hz); 7.69 (m, 2H).

### Example 10.11.

### 1-bromo-4-(bromomethyl)-2-nitrobenzene

### 10.11.1

### (4-bromo-3-nitrophenyl)methanol

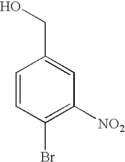

Prepared following the acid reduction method previously described (Method 10F) using 4-bromo-2-nitrobenzoic acid. The product was obtained as a yellow solid.

Yield: 95%

Rf (petroleum ether/ethyl acetate 70/30): 0.48

NMR ¹H (CDCl₃): 2.52 (t, 1H, J=5 Hz); 4.75 (d, 2H, J=5 Hz); 7.42 (d, 1H, J=7.5 Hz); 7.71 (d, 1H, J=7.5 Hz); 7.84 (s, 1H).

### 10.11.2

### 1-bromo-4-(bromomethyl)-2-nitrobenzene

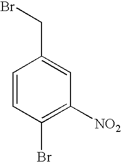

Prepared following the bromination method previously described (Method 10C) using (4-bromo-3-nitrophenyl)methanol (example 10.11.1). The product was obtained as a yellow solid.

Yield: 90%

Rf (petroleum ether/ethyl acetate 95/5): 0.34

NMR ¹H (CDCl₃): 4.48 (s, 2H); 7.49 (dd, 1H, J=7.5 Hz, J=2.0 Hz); 7.71 (d, 1H, J=7.5 Hz); 7.90 (d, 1H, J=2.0 Hz).

### Example 11.

### General Procedure Generate for the Preparation of Protected Tetrazolyl Derivative

### Example 11.1.

### (1-(benzyloxymethyl)-1H-tetrazole

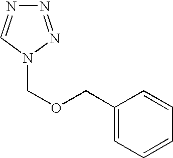

Tetrazole (1eq) and the sodium bis(trimethylsilyl)amide 2M solution (1eq) in tetrahydrofuran were dissolved in anhydrous tetrahydrofuran under argon atmosphere The reaction mixture was stirred at 0° C. for 30 minutes, then benzylchloromethylether (1eq) was added. The mixture was stirred at room temperature for 3 hours. The mixture was then extracted with ethyl acetate. The organic layers were combined and washed with brine, dried over magnesium sulfate and evaporated under reduced pressure. The residue was chromatographed over silica gel (eluent petroleum ether/ethyl acetate 80/20). The product was obtained as a colorless oil.

Yield: 43%

Rf (petroleum ether/ethyl acetate 70/30): 0.48

NMR ¹H (CDCl₃): 4.67 (s, 2H); 5.96 (s, 2H); 7.29-7.40 (m, 5H); 8.61 (s, 1H).

### Example 11.2.

### 1-(1-(benzyloxymethyl)-1H-tetrazol-5-yl)propan-1-ol

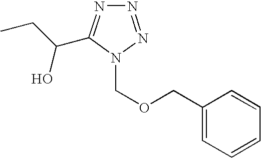

The (1-(benzyloxymethyl)-1H-tetrazole (1eq, example 11.1) est was dissolved in anhydrous tetrahydrofuran under argon atmosphere then collated to −78° C. n-butyllithium (1eq, 1M in hexane) was slowly added, then the reaction mixture was stirred at −78% for 15 minutes. Propionaldehyde was then added and the reaction mixture was stirred at −78° C. for 15 minutes then at temperature ambiante for 45 minutes. An ammonium chloride saturated solution was added and the reaction mixture was extracted with ethyl acetate. The organic layer was washed with brine, dried over magnesium sulfate and evaporated. The residue was chromatographed over silica gel (eluent petroleum ether/ethyl acetate 80/20). The product was obtained as a colorless oil.

Yield: 75%

Rf (petroleum ether/ethyl acetate 70/30): 0.48

NMR ¹H (CDCl₃): 1.04 (t, 3H, J=7.5 Hz); 1.97-2.12 (m, 2H); 3.04 (d, 1H, J=5 Hz); 4.69 (s, 2H); 5.02 (q, 1H, J=5 Hz); 5.92 (s, 2H); 7.35-7.38 (m, 5H).

### Example 11.3.

### methanesulfonate de 1-(1-(benzyloxymethyl)-1H-tetrazol-5-yl)propyle

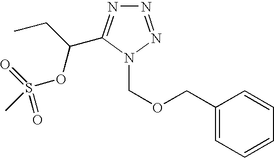

The 1-(1-(benzyloxymethyl)-1H-tetrazol-5-yl)propan-1-ol (1eq, example 11.2) and mesyle chloride (1.2eq) were dissolved in anhydrous dichloromethane under argon atmosphere. The solution was cooled to −10° C., then triethylamine (1.5eq) was added drop by drop. The reaction mixture was stirred at room temperature for 2 hours. The reaction mixture was extracted with dichloromethane and the organic layer was washed with brine, dried over magnesium sulfate and evaporated. The product was obtained as a colorless oil.

Yield: 99%

NMR ¹H (CDCl₃): 1.10 (t, 3H, J=7.5 Hz); 2.28 (m, 2H); 3.08 (s, 3H); 4.71 (s, 2H); 5.87 (t, 1H, J=6.8 Hz); 5.96 (s, 2H); 7.29-7.36 (m, 5H).

### Example 12.

### General Procedure for the Substitution of Imidazolones

Method 12A: To a solution of the appropriate imidazolone (1eq) in anhydrous acetonitrile was added potassium carbonate (2eq). The reaction mixture was stirred for 15 minutes at room temperature, then the brominated derivative was added and the reaction mixture was stirred at 90° C. for 12 hours. The mixture was cooled to room temperature, acidified with a hydrochloric acid solution, and then extracted with ethyl acetate. The organic layers were combined, dried over magnesium sulfate, and evaporated under reduced pressure. The residue was chromatographed over silica gel.

Method 12B: To a solution of the appropriate imidazolone (1eq) and brominated derivative (1.5eq) in N,N-dimethylformamide was added potassium carbonate (2eq). The reaction mixture was stirred for 12 hours at room temperature. The solvent was evaporated under reduced pressure and the residue was taken up in an ethyl acetate/water mixture. The organic layer was separated and washed with brine, and then dried over sodium sulfate, filtered, and concentrated under reduced pressure. The residue was chromatographed over silica gel.

Method 12C: A solution of the brominated derivative (1eq) and the tetrabutylammonium hydrogenosulfate (0.125eq) in toluene was stirred at 90° C. A solution of imidazolone (1.15eq) and potassium hydroxide in water previously stirred for 40 minutes was then added. The biphasic mixture was vigourously stirred at 90° C. for 1 hour. The reaction mixture was then stirred at room temperature for 1 hour. After addition of water, the layers were separated, and the aqueous layer was extracted with toluene. The organic layer was washed with brine, then dried over sodium sulfate, filtered and evaporated. The residue was chromatographed over silica gel.

Method 12D: To a solution of imidazole (1eq) in acetonitrile at 0° C. under nitrogen atmosphere was added sodium hydride (3eq) in portions. The mixture was stirred for 20 minutes, then a solution of the brominated derivative in acetonitrile was added very slowly. The reaction mixture was stirred at room temperature for 12 hours. Water was then added and acetonitrile was evaporated. The mixture was taken up in an water/dichloromethane mixture. The organic layer was separated and washed with brine, then dried over sodium sulfate, filtered and concentrated under reduced pressure. The residue was chromatographed over silica gel.

### Example 12.1.

### 2-butyl-1-[(3′-((1-ethoxycarbonyl-1,1-dimethylmethyl)oxy)biphenyl-4-yl)methyl]-4-spirocyclopentyl-1H-imidazol-5(4H)-one and 1-[(6′-bromo-3′-((1-ethoxycarbonyl-1,1-dimethylmethyl)oxy)biphenyl-4-yl)methyl]-2-butyl-4-spirocyclopentyl-1H-imidazol-5(4H)-one

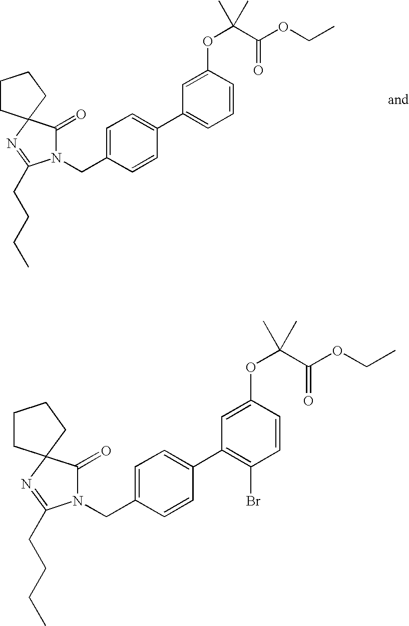

Prepared following the general procedure previously described (Method 12A) using 2-butyl-4-spirocyclopentyl-1H-imidazol-5(4H)-one (example 3.4) and a mixture of ethyl 2-((4′-bromomethylbiphenyl-3-yl)oxy)-2-methylpropanoate and ethyl 2-((6-bromo-4′-bromomethylbiphenyl-3-yl)oxy)-2-methylpropanoate (example 6.2). The products were chromatographed over silica gel (eluent dichloromethane/methanol 98/2, then cyclohexane/acetone 85/15). The products were obtained as a colorless oil (mixture of 2 compounds).

Yield: 69%

Rf (dichloromethane/methanol 98/2): 0.34

IR: νCO: 1732 cm⁻¹; 1633 cm⁻¹

NMR ¹H (CDCl₃) (derivative non-brominated on the aromatic cycle): 0.87 (t, 3H, J=7.3 Hz); 1.23 (t, 3H, J=7.3 Hz); 1.33 (sext, 2H, J=7.6 Hz); 1.57 (quint, 2H, J=7.6 Hz); 1.64 (s, 6H); 1.82-2.05 (m, 8H); 2.34 (t, 2H, J=8.2 Hz); 4.26 (q, 2H, J=6.7 Hz); 4.72 (s, 2H); 6.82 (ddd, 1H, J=8.2 Hz, J=2.3 Hz, J=1.2 Hz); 7.09 (t, 1H, J=2.1 Hz); 7.19-7.23 (m, 3H); 7.31 (t, 1H, J=7.9 Hz); 7.52 (d, 2H, J=8.2 Hz) NMR ¹H (CDCl₃) (derivative brominated on the aromatic cycle): 0.87 (t, 3H, J=7.3 Hz); 1.23 (t, 3H, J=7.3 Hz); 1.33 (sext, 2H, J=7.6 Hz); 1.57 (quint, 2H, J=7.6 Hz); 1.64 (s, 6H); 1.82-2.05 (m, 8H); 2.34 (t, 2H, J=8.2 Hz); 4.26 (q, 2H, J=6.7 Hz); 4.74 (s, 2H); 6.70 (dd, 1H, J=8.8 Hz, J=2.9 Hz); 7.09 (t, 1H, J=2.1 Hz); 7.19-7.23 (m, 2H); 7.36 (d, 1H, J=8.5 Hz); 7.50 (d, 2H, J=7 Hz).

### Example 12.2.

### 2-butyl-1-[(4′-((1-ethoxycarbonyl-1,1-dimethylmethyl)oxy)biphenyl-4-yl)methyl]-4-spirocyclopentyl-1H-imidazol-5(4H)-one

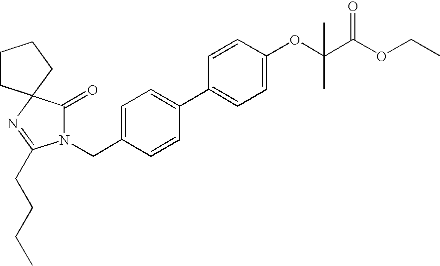

Prepared following the general procedure previously described (Method 12A) using 2-butyl-4-spirocyclopentyl-1H-imidazol-5(4H)-one (example 3.4) and ethyl 2-((4′-bromomethylbiphenyl-4-yl)oxy)-2-methylpropanoate (example 6.3). The product was chromatographed over silica gel (eluent cyclohexane/ethyl acetate 9/1 to 6/4). The product was obtained as a colorless oil.

Yield: 35%

Rf (cyclohexane/ethyl acetate 5/5): 0.70

IR: νCO: 1730 cm⁻¹

NMR ¹H (CDCl₃): 0.86 (t, 3H, J=7.2 Hz); 1.27 (t, 3H, J=6.9 Hz); 1.25-1.40 (m, 2H); 1.52-1.65 (m, 2H); 1.64 (s, 6H); 1.75-1.90 (m, 2H); 1.90-2.10 (m, 6H); 2.34 (t, 2H, J=7.2 Hz); 4.22-4.29 (q, 2H, J=7.2 Hz); 4.71 (s, 2H); 6.91 (d, 2H, J=8.8 Hz); 7.20 (d, 2H, J=8.2 Hz); 7.45 (d, 2H, J=8.9 Hz); 7.51 (d, 2H, J=8.2 Hz).

### Example 12.3.

### 2-butyl-1-[2-(4-((1-ethoxycarbonyl-1,1-dimethylmethyl)oxy)phenyl)ethyl]-4-spirocyclopentyl-1H-imidazol-5(4H)-one

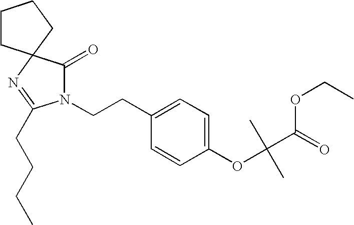

Prepared following the general procedure previously described (Method 12A) using 2-butyl-4-spirocyclopentyl-1H-imidazol-5(4H)-one (example 3.4) and ethyl 2-(4-(2-bromoethyl)phenoxy)-2-methylpropanoate (example 5.3). The product was chromatographed over silica gel (eluent dichloromethane/methanol 7/3). The product was obtained as a colorless oil.

Yield: 9%

Rf (cyclohexane/ethyl acetate 6/4): 0.30

IR: νCO: 1730 cm⁻; 1629 cm⁻

NMR ¹H (CDCl₃): 0.91 (t, 3H, J=7.3 Hz); 1.27 (t, 3H, J=7.3 Hz); 1.34 (sext, 2H, J=7.3 Hz); 1.45-1.64 (m, 2H); 1.59 (s, 6H); 1.65-1.83 (m, 2H); 1.83-2.00 (m, 6H); 2.07 (t, 2H, J=7.9 Hz); 2.83 (t, 2H, J=7 Hz); 3.63 (t, 2H, J=7Hz); 4.25 (q, 2H, J=7 Hz); 6.78 (d, 2H, J=8.5 Hz); 7.00 (d, 2H, J=8.5 Hz).

### Example 12.4.

### 2-butyl-1-[(2′-((1-ethoxycarbonyl-1,1-dimethylmethyl)oxy)biphenyl-4-yl)methyl]-4-spirocyclopentyl-1H-imidazol-5(4H)-one and 1-[(5′-bromo-2′-((1-ethoxycarbonyl-1,1-dimethylmethyl)oxy)biphenyl-4-yl)methyl]-2-butyl-4-spirocyclopentyl-1H-imidazol-5(4H)-one

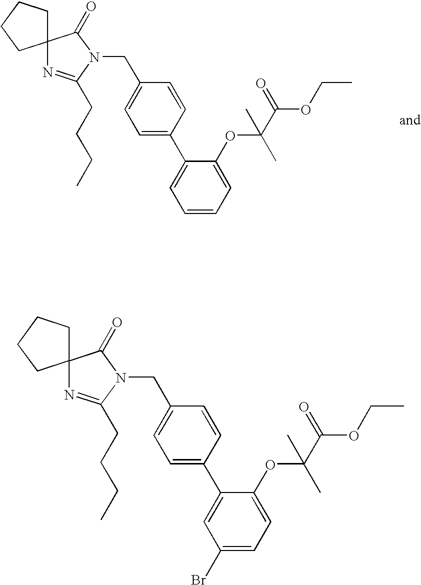

Prepared following the general procedure previously described (Method 12A) using 2-butyl-4-spirocyclopentyl-1H-imidazol-5(4H)-one (example 3.4) and a mixture of ethyl 2-((4′-bromomethylbiphenyl-2-yl)oxy)-2-methylpropanoate and ethyl 2-((5-bromo-4′-bromomethylbiphenyl-2-yl)oxy)-2-methylpropanoate (example 6.1). The products were chromatographed over silica gel (eluent dichloromethane/ethyl acetate 9/1). The product was obtained as a colorless oil (mixture of 2 compounds).

Total yield: 48%

Rf (dichloromethane/ethyl acetate 9/1): 0.30

IR: νCO: 1737 cm⁻¹; 1633 cm⁻¹

NMR ¹H (CDCl₃) (derivative non-brominated on the aromatic cycle): 0.85 (t, 3H, J=7.3 Hz); 1.23 (t, 3H, J=7 Hz); 1.33 (sext, 2H, J=7.3 Hz); 1.40 (s, 6H); 1.57 (quint, 2H, J=7 Hz); 1.81-2.03 (m, 8H); 2.34 (t, 2H, J=7.9 Hz); 4.23 (q, 2H, J=7 Hz); 4.74 (s, 2H); 6.87 (d, 1H, J=8.2 Hz); 7.07 (t, 1H, J=7.3 Hz); 7.15-7.21 (d, 2H, J=7.9 Hz); 7.20-7.26 (dd, 1H, J=7.9 Hz, J=1.8 Hz); 7.29-7.34 (dd, 1H, J=7.6 Hz, J=1.8 Hz); 7.52 (d, 2H, J=8.2 Hz).

NMR ¹H (CDCl₃) (derivative brominated on the aromatic cycle): 0.90 (t, 3H, J=7.3 Hz); 1.23 (t, 3H, J=7 Hz); 1.30-1.43 (m, 2H); 1.40 (s, 6H); 1.60-1.72 (quint, 2H, J=7.9 Hz); 1.81-2.03 (m, 8H); 2.43 (t, 2H, J=7.9 Hz); 4.23 (q, 2H, J=7 Hz); 4.88 (s, 2H); 6.95 (d, 1H, J=8.2 Hz); 7.07 (d, 2H, J=7.9 Hz); 7.38 (dd, 1H, J=8.8 Hz, J=2.6 Hz); 7.45 (d, 1H, J=2.6 Hz); 7.57 (d, 2H, J=8.2 Hz).

### Example 12.5.

### 2-butyl-1-[[4-[(3-((1-ethoxycarbonyl-1,1-dimethylmethyl)oxy)phenyl)carbonyl]phenyl]methyl]-4-spirocyclopentyl-1H-imidazol-5(4H)-one

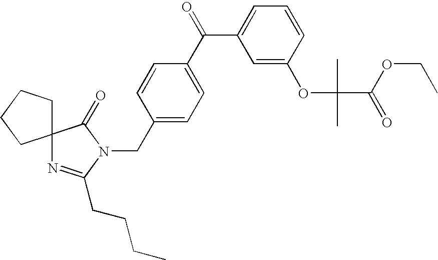

Prepared following the general procedure previously described (Method 12A) using 2-butyl-4-spirocyclopentyl-1H-imidazol-5(4H)-one (example 3.4) and (3-((1-ethoxycarbonyl-1,1-dimethylmethyl)oxy)phenyl)(4-(bromomethyl)phenyl)methanone (example 7.2). The product was chromatographed over silica gel (eluent dichloromethane/ethyl acetate 8/2). The product was obtained as a colorless oil.

Yield: 14%

Rf (dichloromethane/ethyl acetate 8/2): 0.30

IR: νCO: 1729 cm⁻¹; 1659 cm⁻¹

NMR ¹H (CDCl₃): 0.88 (t, 3H, J=7.3 Hz); 1.25 (t, 3H, J=7.3 Hz); 1.34 (sext, 2H, J=7.6 Hz); 1.50-1.68 (quint, 2H, J=7.9 Hz); 1.63 (s, 6H); 1.70-2.05 (m, 8H); 2.32 (t, 2H, J=7.3 Hz); 4.23 (q, 2H, J=7 Hz); 4.77 (s, 2H); 7.08 (d, 1H, J=7.6 Hz); 7.25-7.42 (m, 5H); 7.78 (d, 2H, J=7.9 Hz).

### Example 12.6.

### 2-butyl-1-[[4-[(2-((1-ethoxycarbonyl-1,1-dimethylmethyl)oxy)phenyl)carbonyl]phenyl]methyl]-4-spirocyclopentyl-1H-imidazol-5(4H)-one

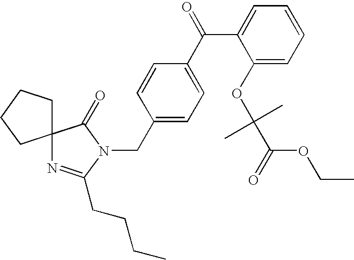

Prepared following the general procedure previously described (Method 12A) using 2-butyl-4-spirocyclopentyl-1H-imidazol-5(4H)-one (example 3.4) and (2-((1-ethoxycarbonyl-1,1-dimethylmethyl)oxy)phenyl)(4-(bromomethyl)phenyl)methanone (example 7.1). The product was chromatographed over silica gel (eluent dichloromethane/methanol 98/2). The product was obtained as a colorless oil.

Yield: 81%

Rf (dichloromethane/methanol 98/2): 0.30

IR: νCO: 1729 cm⁻¹; 1653 cm⁻¹; 1633 cm⁻¹

NMR ¹H (CDCl₃): 0.87 (t, 3H, J=7.3 Hz); 1.23 (t, 3H, J=7 Hz); 1.28-1.40 (m, 2H); 1.31 (s, 6H); 1.58 (quint, 2H, J=7.6 Hz); 1.80-2.01 (m, 8H); 2.27 (t, 2H, J=7.6 Hz); 4.20 (q, 2H, J=7 Hz); 4.74 (s, 2H); 6.75 (d, 1H, J=8.2 Hz); 7.08 (t, 1H, J=7.3 Hz); 7.21 (d, 2H, J=8.2 Hz); 7.37 (td, 1H, J=8.7 Hz, J=1.7 Hz); 7.44 (dd, 1H, J=7.3 Hz J=1.7 Hz); 7.81 (d, 2H, J=8.2 Hz).

### Example 12.7.

### 2-butyl-1-[2-(3-((1-ethoxycarbonyl-1,1-dimethylmethyl)oxy)phenyl)ethyl]-4-spirocyclopentyl-1H-imidazol-5(4H)-one

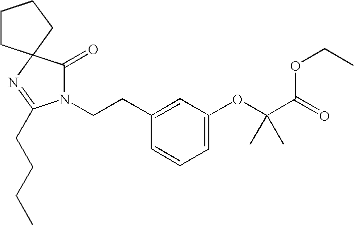

Prepared following the general procedure previously described (Method 12A) using 2-butyl-4-spirocyclopentyl-1H-imidazol-5(4H)-one (example 3.4) and ethyl 2-(3-(2-bromoethyl)phenoxy)-2-methylpropanoate (example 5.2). The product was chromatographed over silica gel (eluent dichloromethane/ethyl acetate 5/5, then dichloromethane/methanol 9/1). The product was obtained as a colorless oil.

Yield: 18%

Rf (dichloromethane/ethyl acetate 5/5): 0.45

IR: νCO: 1726 cm⁻¹; 1630 cm⁻¹

NMR ¹H (CDCl₃): 0.91 (t, 3H, J=7.3 Hz); 1.26 (t, 3H, J=7 Hz); 1.25-1.39 (sext, 2H, J=7.6 Hz); 1.50-1.61 (m, 8H); 1.65-1.85 (m, 2H); 1.85-2.02 (m, 6H); 2.08 (t, 2H, J=7.3 Hz); 2.82 (t, 2H, J=7.3 Hz); 3.64 (t, 2H, J=7 Hz); 4.24 (q, 2H, J=7 Hz); 6.67-6.69 (d, 1H, J=6.4 Hz); 6.69 (s, 1H); 6.75 (d, 1H, J=7.6 Hz); 7.16 (t, 1H, J=7.6 Hz)

### Example 12.8.

### 2-butyl-1-[[4-[(4-((1-ethoxycarbonyl-1,1-dimethylmethyl)oxy)phenyl)carbonyl]phenyl]methyl]-4-spirocyclopentyl-1H-imidazol-5(4H)-one

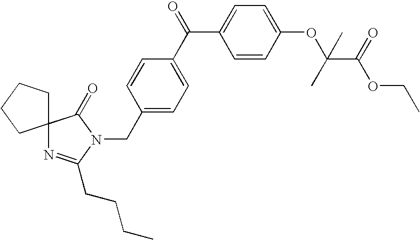

Prepared following the general procedure previously described (Method 12A) using 2-butyl-4-spirocyclopentyl-1H-imidazol-5(4H)-one (example 3.4) and (4-((1-ethoxycarbonyl-1,1-dimethylmethyl)oxy)phenyl)(4-(bromomethyl)phenyl)methanone (example 7.3). The product was chromatographed over silica gel (eluent dichloromethane/methanol 98/2). The product was obtained as a colorless oil.

Yield: 79%

Rf (dichloromethane/methanol 98/2): 0.30

IR: νCO: 1729 cm⁻¹; 1653 cm⁻¹; 1633 cm⁻¹

NMR ¹H (CDCl₃): 0.87 (t, 3H, J=7.3 Hz); 1.23 (t, 3H, J=7 Hz); 1.23-1.40 (sext, 2H, J=7.6 Hz); 1.50-1.66 (quint, 2H, J=7.6 Hz); 1.67 (s, 6H); 1.75-2.06 (m, 8H); 2.32 (t, 2H, J=7.6 Hz); 4.23 (q, 2H, J=7 Hz); 4.76 (s, 2H); 6.85 (d, 2H, J=9 Hz); 7.25 (d, 2H, J=8.2 Hz); 7.75 (d, 4H, J=7.2 Hz).

### Example 12.9.

### 2-butyl-1-[2-(2-((1-ethoxycarbonyl-1,1-dimethylmethyl)oxy)phenyl)ethyl]-4-spirocyclopentyl-1H-imidazol-5(4H)-one

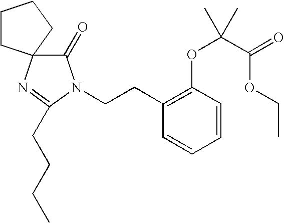

Prepared following the general procedure previously described (Method 12A) using 2-butyl-4-spirocyclopentyl-1H-imidazol-5(4H)-one (example 3.4) and ethyl 2-(2-(2-bromoethyl)phenoxy)-2-methylpropanoate (example 5.1). The product was chromatographed over silica gel (eluent dichloromethane/methanol 97/3). The product was obtained as a colorless oil.

Yield: 20%

Rf (dichloromethane/methanol 97/3): 0.30

IR: νCO: 1733 cm⁻¹; 1624 cm⁻¹

NMR ¹H (CDCl₃): 0.91 (t, 3H, J=7.3 Hz); 1.23 (t, 3H, J=7.3 Hz); 1.35 (sext, 2H, J=7.6 Hz); 1.58 (quint, 2H, J=8.2 Hz); 1.67 (s, 6H); 1.73-1.94 (m, 8H); 2.21 (t, 2H, J=7.3 Hz); 2.91 (t, 2H, J=7.3 Hz); 3.69 (t, 2H, J=7 Hz); 4.23 (q, 2H, J=7 Hz); 6.64 (d, 1H, J=7.9 Hz); 6.87 (t, 1H, J=7.3 Hz); 7.03 (dd, 1H, J=7.3 Hz, J=1.4 Hz); 7.11 (td, 1H, J=8.5 Hz, J=1.7 Hz).

### Example 12.10.

### 2-butyl-1-[[4-[(3-((1-ethoxycarbonyl-1,1-dimethylmethyl)oxy)phenyl)carbonyl]phenyl]methyl]-4-spirocyclohexyl-1H-imidazol-5(4H)-one

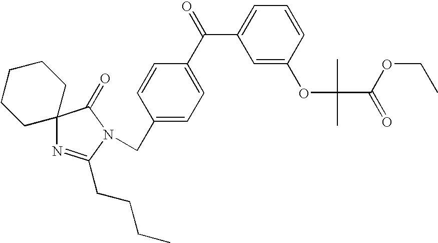

Prepared following the general procedure previously described (Method 12A) using 2-butyl-4-spirocyclohexyl-1H-imidazol-5(4H)-one (example 3.5) and (3-((1-ethoxycarbonyl-1,1-dimethylmethyl)oxy)phenyl)(4-(bromomethyl)phenyl)methanone (example 7.2). The product was chromatographed over silica gel (eluent dichloromethane/methanol 98/2). The product was obtained as a yellowish oil.

Yield: 78%

Rf (dichloromethane/methanol 98/2): 0.30

IR: νCO: 1,728 cm⁻¹; 1,660 cm⁻¹; 1,636 cm⁻¹

NMR ¹H (CDCl₃): 0.88 (t, 3H, J=7.3 Hz); 1.24 (t, 3H, J=7.3 Hz); 1.36 (sext, 2H, J=7.6 Hz); 1.52-1.80 (m, 12H); 1.62 (s, 6H); 2.33 (t, 2H, J=7.6 Hz); 4.22 (q, 2H, J=7 Hz); 4.75 (s, 2H); 7.08 (d, 1H, J=7.6 Hz); 7.24-7.28 (m, 3H); 7.33-7.40 (m, 2H); 7.78 (d, 2H, J=8.5 Hz).

### Example 12.11.

### 2-butyl-4,4-dimethyl-1-[(3′-((1-ethoxycarbonyl-1,1-dimethylmethyl)oxy)biphenyl-4-yl)methyl]-1H-imidazol-5(4H)-one and 1-[(6′-bromo-3′-((1-ethoxycarbonyl-1,1-dimethylmethyl)oxy)biphenyl-4-yl)methyl]-2-butyl-4,4-dimethyl-1H-imidazol-5(4H)-one

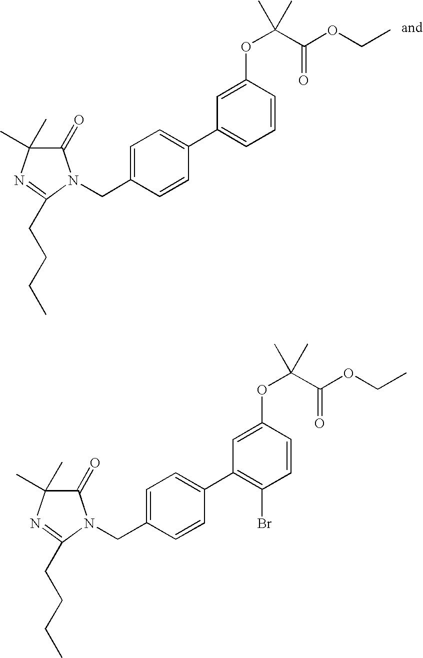

Prepared following the general procedure previously described (Method 12A) using 2-butyl-4,4-dimethyl-1H-imidazol-5(4H)-one (example 3.6) and a mixture of ethyl 2-((4′-bromomethylbiphenyl-3-yl)oxy)-2-methylpropanoate and ethyl 2-((6-bromo-4′-bromomethylbiphenyl-3-yl)oxy)-2-methylpropanoate (example 6.2). The products were chromatographed over silica gel (eluent dichloromethane/methanol 98/2). The products were obtained as a yellowish oil (mixture of 2 compounds).

Yield: 57%

Rf (dichloromethane/ethyl acetate 5/5): 0.60

IR: νCO: 1728 cm⁻¹; 1635 cm⁻¹

NMR ¹H (CDCl₃) (derivative non-brominated on the aromatic cycle): 0.88 (t, 3H, J=7.3 Hz); 1.25 (t, 3H, J=7 Hz); 1.35 (sext, 2H, J=7 Hz); 1.39 (s, 6H); 1.61-1.68 (m, 2H); 1.64 (s, 6H); 2.34 (t, 2H, J=7.6 Hz); 4.26 (q, 2H, J=6.7 Hz); 4.71 (s, 2H); 6.82 (d, 1H, J=8.2 Hz); 7.09 (s, 1H); 7.19-7.37 (m, 4H); 7.53 (d, 2H, J=8.2 Hz).

NMR ¹H (CDCl₃) (derivative brominated on the aromatic cycle): 0.88 (t, 3H, J=7.3 Hz); 1.25 (t, 3H, J=7 Hz); 1.35 (sext, 2H, J=7 Hz); 1.39 (s, 6H); 1.61-1.68 (m, 2H); 1.64 (s, 6H); 2.34 (t, 2H, J=7.6 Hz); 4.26 (q, 2H, J=6.7 Hz); 4.73 (s, 2H),; 6.70 (dd, 1H, J=8.2 Hz, J=2.8 Hz); 7.09 (s, 1H); 7.19-7.37 (m, 3H); 7.53 (d, 2H, J=8.2 Hz).

### Example 12.12.

### 2-butyl-4,4-dimethyl-1-[[4-[(3-((1-ethoxycarbonyl-1,1-dimethylmethyl)oxy)phenyl)carbonyl]phenyl]methyl]-1H-imidazol-5(4H)-one

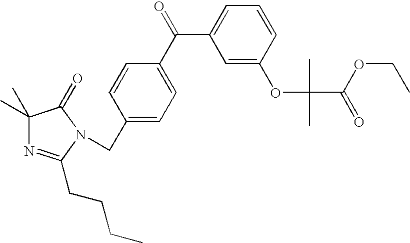

Prepared following the general procedure previously described (Method 12A) using 2-butyl-4,4-dimethyl-1H-imidazol-5(4H)-one (example 3.6) and (3-((1-ethoxycarbonyl-1,1-dimethylmethyl)oxy)phenyl)(4-(bromomethyl)phenyl)methanone (example 7.2). The product was chromatographed over silica gel (eluent dichloromethane/methanol 98/2). The product was obtained as a colorless oil.

Yield: 34%

Rf (dichloromethane/methanol 98/2): 0.25

IR: νCO: 1731 cm⁻¹; 1686 cm⁻¹; 1636 cm⁻¹

NMR ¹H (CDCl₃): 0.80 (t, 3H, J=7.3 Hz); 1.16 (t, 3H, J=7 Hz); 1.25 (sext, 2H, J=7.9 Hz); 1.31 (s, 6H); 1.51-1.61 (m, 2H); 1.54 (s, 6H); 2.26 (t, 2H, J=7.3 Hz); 4.14 (q, 2H, J=7 Hz); 4.70 (s, 2H); 7.01 (dd, 1H, J=7.3 Hz, J=2.1 Hz); 7.20-7.33 (m, 5H); 7.71 (d, 2H, J=8.2 Hz).

### Example 12.13.

### 2-butyl-1-[[4-[(3-((1-ethoxycarbonyl-1,1-dimethylmethyl)oxy)phenyl)carbonyl]phenyl]methyl]-4-phenyl-1H-imidazol-5(4H)-one

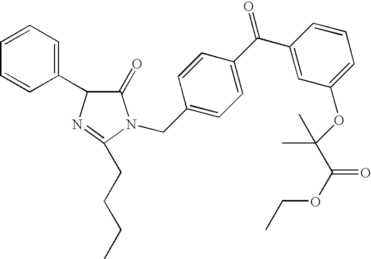

Prepared following the general procedure previously described (Method 12A) using 2-butyl-4-phenyl-imidazol-5(4H)-one (example 3.7) and (3-((1-ethoxycarbonyl-1,1-dimethylmethyl)oxy)phenyl )(4-(bromomethyl)phenyl)methanone (example 7.2). The product was chromatographed over silica gel (eluent dichloromethane/methanol 98/2). The product was obtained as a brown oil.

Yield: 83%

Rf (dichloromethane/methanol 98/2): 0.34

IR: νCO: 1734 cm⁻¹; 1657 cm⁻¹; 1666 cm⁻¹

NMR ¹H (CDCl₃): 0.84 (t, 3H, J=7.3 Hz); 1.15-1.29 (m, 5H); 1.46 (quint, 2H, J=7.3 Hz); 1.57 (s, 6H); 2.28 (t, 2H, J=7.6 Hz); 3.25-3.55 (m, 2H); 4.26 (q, 2H, J=6.7 Hz); 5.29 (s, 1H); 7.01-7.11 (m, 2H); 7.27-7.45 (m, 7H); 7.60-7.82 (m, 4H).

### Example 12.14.

### 1-[(3′-((1-tert-butyloxycarbonyl-1,1-dimethylmethyl)oxy)biphenyl-4-yl)methyl]-2-propyl-4-spirocyclopentyl-1H-imidazol-5(4H)-one

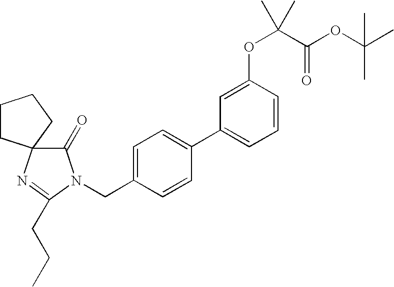

Prepared following the general procedure previously described (Method 12B) using 2-propyl-4-spirocyclopentyl-1H-imidazol-5(4H)-one (example 3.3) and tert-butyl 2-((4′-bromomethylbiphenyl-3-yl)oxy)-2-methylpropanoate (example 6.4). The product was chromatographed over silica gel (elution gradient cyclohexane/ethyl acetate 8/2 to 1/1). The product was obtained as a viscous yellow oil.

Yield: 38%

NMR ¹H (CDCl₃): 0.85 (t, 3H, J=7.3 Hz); 1.45 (s, 9H); 1.62 (s, 6H); 1.68 (sext, 2H, J=7.6 Hz); 1.85 (m, 2H); 1.92-2.10 (m, 6H); 2.32 (q, 2H, J=7.3 Hz); 4.73 (s, 2H); 6.85 (dd, 1H, J=8.2 Hz, J=2 Hz); 7.12 (s, 1H); 7.18-7.25 (m, 3H); 7.32 (t, 1H, J=7.9 Hz); 7.55 (d, 2H, J=8 Hz).

### Example 12.15.

### 1-[(3′-((1-tert-butyloxycarbonyl-1,1-dimethylmethyl)oxy)biphenyl-4-yl)methyl]-2-ethyl-4-spirocyclopentyl-1H-imidazol-5(4H)-one

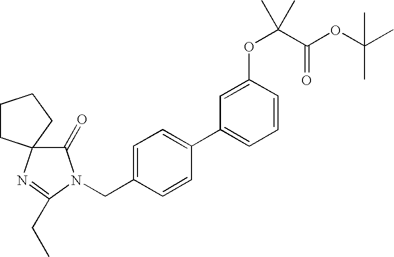

Prepared following the general procedure previously described (Method 12B) using 2-ethyl-4-spirocyclopentyl-1H-imidazol-5(4H)-one (example 3.2) and tert-butyl 2-((4′-bromomethylbiphenyl-3-yl)oxy)-2-methylpropanoate (example 6.4). The product was chromatographed over silica gel (elution gradient cyclohexane/ethyl acetate 8/2 to 1/1). The product was obtained as a viscous yellow oil.

Yield: 70%

NMR ¹H (CDCl₃): 1.20 (t, 3H, J=7.3 Hz); 1.45 (s, 9H); 1.62 (s, 6H); 1.85 (m, 2H); 1.94-2.08 (m, 6H); 2.39 (q, 2H, J=7.3 Hz); 4.72 (s, 2H); 6.83 (dd, 1H, J=8.2 Hz, J=2 Hz); 7.09 (s, 1H); 7.15-7.25 (m, 3H); 7.30 (t, 1H, J=7.9 Hz); 7.52 (d, 2H, J=8 Hz).

### Example 12.16.

### 1-[(3′-((1-tert-butyloxycarbonyl-1,1-dimethylmethyl)oxy)biphenyl-4-yl)methyl]-2-methyl-4-spirocyclopentyl-1H-imidazol-5(4H)-one

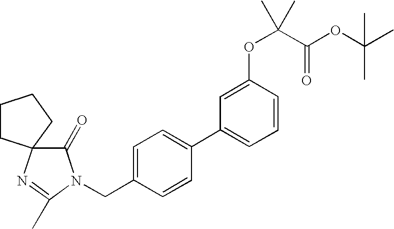

Prepared following the general procedure previously described (Method 12B) using 2-methyl-4-spirocyclopentyl-1H-imidazol-5(4H)-one (example 3.1) and tert-butyl 2-((4′-bromomethylbiphenyl-3-yl)oxy)-2-methylpropanoate (example 6.4). The product was chromatographed over silica gel (elution gradient cyclohexane/ethyl acetate 95/5 to 20/80). The product was obtained as a viscous yellow oil.

Yield: 30%

NMR ¹H (CDCl₃): 1.45 (s, 9H); 1.60 (s, 6H); 1.80 (m, 2H); 1.90-2.08 (m, 6H); 2.11 (s, 3H); 4.72 (s, 2H); 6.83 (dd, 1H, J=8.2 Hz, J=2 Hz); 7.09 (s, 1H); 7.19 (d, 1H, J=8 Hz); 7.23 (d, 2H, J=8 Hz); 7.30 (t, 1H, J=7.9 Hz); 7.52 (d, 2H, J=8 Hz).

### Example 12.17.

### 2-butyl-1-[(3′-((1-ethoxycarbonyl-1,1-dimethylmethyl)oxy)biphenyl-4-yl)methyl]-4-phenyl-1H-imidazol-5(4H)-one

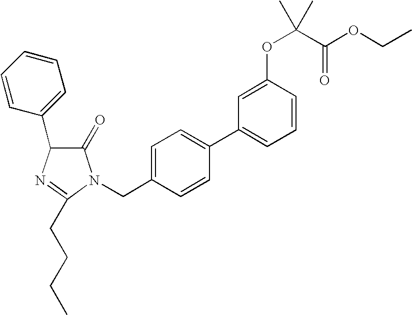

Prepared following the general procedure previously described (Method 12A) using 2-butyl-4-phenyl-1H-imidazol-5(4H)-one (example 3.7) and a mixture of ethyl 2-((4′-bromomethylbiphenyl-3-yl)oxy)-2-methylpropanoate and ethyl 2-((6-bromo-4′-bromomethyl-biphenyl-3-yl)oxy)-2-methylpropanoate (example 6.2). The product was chromatographed over silica gel (eluent dichloromethane/methanol 98/2). The product was obtained as a yellowish oil.

Yield: 52%

Rf (dichloromethane/methanol 95/5): 0.30

IR: νCO: 1732 cm⁻¹; 1642 cm⁻¹

NMR ¹H (CDCl₃): 0.82 (t, 3H, J=7.3 Hz); 1.10-1.22 (sext, 2H, J=7.3 Hz); 1.23 (t, 3H, J=7 Hz); 1.40-1.51 (quint, 2H, J=7.3 Hz); 1.64 (s, 6H); 2.34 (m, 2H); 3.33-3.52 (m, 2H); 4.26 (q, 2H, J=6.7 Hz); 5.32 (s, 1H); 6.77-6.81 (dd, 1H, J=8.2 Hz, J=1.8 Hz); 7.09 (s, 1H); 7.15-7.45 (m, 9H); 7.75 (d, 2H, J=8.2 Hz).

### Example 12.18.

### 2-butyl-1-[[4-[(4-((1-ethoxycarbonyl-1,1-dimethylmethyl)oxy)phenyl)carbonyl]phenyl]methyl]-4-phenyl-1H-imidazol-5(4H)-one

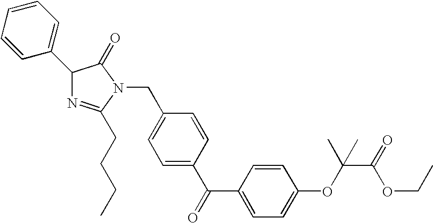

Prepared following the general procedure previously described (Method 12A) using 2-butyl-4-phenyl-1H-imidazol-5(4H)-one (example 3.7) and (4-((1-ethoxycarbonyl-1,1-dimethylmethyl)oxy)phenyl)(4-(bromomethyl)phenyl)methanone (example 7.3). The product was chromatographed over silica gel (eluent dichloromethane/methanol 98/2). The product was obtained as a colorless oil.

Yield: 17%

Rf (dichloromethane/methanol 98/2): 0.25

IR: νCO: 1735 cm⁻¹; 1647 cm⁻¹; 1599 cm⁻¹

NMR ¹H (CDCl₃): 0.82 (t, 3H, J=7 Hz); 1.17 (sext, 2H, J=7.9 Hz); 1.23 (t, 3H, J=7.3 Hz); 1.43 (quint, 2H, J=7.3 Hz); 1.66 (s, 6H); 2.27-2.33 (t, 2H, J=7.6 Hz); 3.33-3.53 (m, 2H); 4.23 (q, 2H, J=7 Hz); 5.33 (s, 1H); 6.83 (d, 2H, J=8.8 Hz); 7.25-7.41 (m, 5H); 7.60 (d, 2H, J=7.9 Hz); 7.70 (d, 4H, J=8.8 Hz).

### Example 12.19.

### 2-butyl-1-[(3′-((1-ethoxycarbonyl-1,1-dimethylmethyl)oxy)biphenyl-4-yl)methyl]-4-spirocyclohexyl-1H-imidazol-5(4H)-one and 1-[(6′-bromo-3′-((1-ethoxycarbonyl-1,1-dimethylmethyl)oxy)biphenyl-4-yl)methyl]-2-butyl-4-spirocyclohexyl-1H-imidazol-5(4H)-one

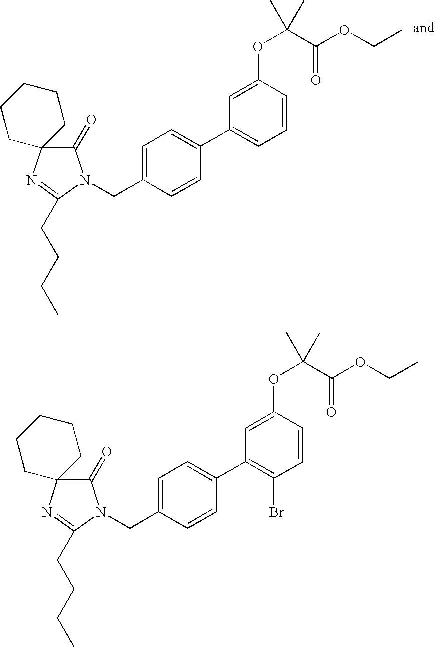

Prepared following the general procedure previously described (Method 12A) using 2-butyl-4-spirocyclohexyl-1H-imidazol-5(4H)-one (example 3.5) and a mixture of ethyl 2-((4′-bromomethylbiphenyl-3-yl)oxy)-2-methylpropanoate and ethyl 2-((6-bromo-4′-bromomethyl-biphenyl-3-yl )oxy)-2-methylpropanoate (example 6.2). The products were chromatographed over silica gel (eluent dichloromethane/methanol 98/2). The products were obtained as a yellowish oil (mixture of 2 compounds).

Yield: 83%

Rf (dichloromethane/ethyl acetate 5/5): 0.60

IR: νCO: 1727 cm⁻¹; 1634 cm⁻¹

NMR ¹H (CDCl₃) (derivative non-brominated on the aromatic cycle): 0.87 (t, 3H, J=7.3 Hz); 1.25 (t, 3H, J=7.3 Hz); 1.26-1.38 (sext, 2H, J=7.6 Hz); 1.42-1.88 (m, 12H); 1.64 (s, 6H); 2.33-2.40 (t, 2H, J=8.2 Hz); 4.25 (q, 2H, J=6.7 Hz); 4.71 (s, 2H); 6.81 (ddd, 1H, J=9.1 Hz, J=2.9 Hz, J=1.5 Hz); 7.08 (t, 1H, J=2.1 Hz); 7.17-7.24 (m, 3H); 7.31 (t, 1H, J=7.9 Hz); 7.52 (d, 2H, J=8.2 Hz).

NMR ¹H (CDCl₃) (derivative brominated on the aromatic cycle): 0.87 (t, 3H, J=7.3 Hz); 1.24 (t, 3H, J=7.3 Hz); 1.26-1.38 (sext, 2H, J=7.6 Hz); 1.42-1.88 (m, 12H); 1.62 (s, 6H); 2.33-2.40 (t, 2H, J=8.2 Hz); 4.25 (q, 2H, J=6.7 Hz); 4.73 (s, 2H); 6.68-6.72 (dd, 1H, J=8.8 Hz, J=2.9 Hz); 7.09 (t, 1H, J=2.1 Hz); 7.17-7.24 (m, 2H); 7.36 (d, 1H, J=8.5 Hz); 7.50 (d, 2H, J=7 Hz).

### Example 12.20.

### 1-[(5′-bromo-2′-((1-ethoxycarbonyl-1,1-dimethylmethyl)oxy)biphenyl-4-yl)methyl]-2-butyl-4-spirocyclohexyl-1H-imidazol-5(4H)-one and 2-butyl-1-[(2′-((1-ethoxycarbonyl-1,1-dimethylmethyl)oxy)biphenyl-4-yl)methyl]-4-spirocyclohexyl-1H-imidazol-5(4H)-one

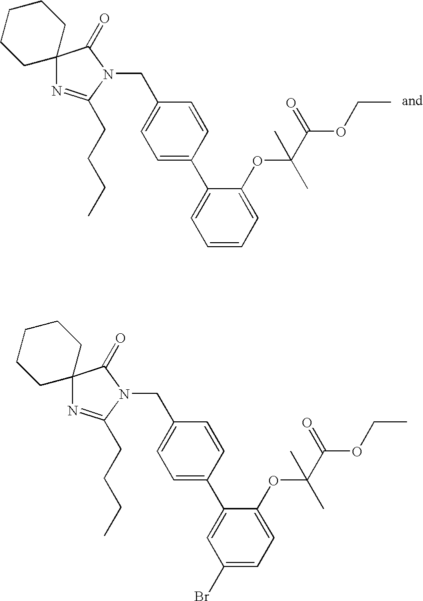

Prepared following the general procedure previously described (Method 12A) using 2-butyl-4-spirocyclohexyl-1H-imidazol-5(4H)-one (example 3.5) and a mixture of ethyl 2-((4′-bromomethylbiphenyl-2-yl)oxy)-2-methylpropanoate and ethyl 2-((5-bromo-4′-bromomethyl-biphenyl-2-yl)oxy)-2-methylpropanoate (example 6.1). The products were chromatographed over silica gel (eluent dichloromethane/methanol 95/5). The products were obtained as a colorless oil (mixture of 2 compounds).

Total yield: 48%

Rf (dichloromethane/methanol 98/2): 0.25

IR: νCO: 1728 cm⁻¹; 1635 cm⁻¹

NMR ¹H (CDCl₃) (derivative non-brominated on the aromatic cycle): 0.88 (t, 3H, J=7.3 Hz); 1.28 (t, 3H, J=7 Hz); 1.34 (sext, 2H, J=7.6 Hz); 1.42 (s, 6H); 1.45-1.81 (m, 12H); 2.36 (m, 2H); 4.24 (q, 2H, J=7.3 Hz); 4.73 (s, 2H); 6.88 (dd, 1H, J=8.2 Hz, J=0.9 Hz); 7.07 (td, 1H, J=7.3 Hz, J=0.9 Hz); 7.18 (d, 2H, J=7.9 Hz); 7.22 (m, 1 H); 7.31 (dd, 1H, J=7.3 Hz, J=1.5 Hz); 7.53 (d, 2H, J=8.2 Hz).

NMR ¹H (CDCl₃) (derivative brominated on the aromatic cycle): 0.88 (t, 3H, J=7.3 Hz); 1.28 (t, 3H, J=7 Hz); 1.34 (sext, 2H, J=7.6 Hz); 1.42 (s, 6H); 1.45-1.81 (m, 12H); 2.36 (m, 2H); 4.24 (q, 2H, J=7.3 Hz); 4.73 (s, 2H); 6.76 (d, 1H, J=8.2 Hz); 7.18 (d, 2H, J=7.9 Hz); 7.22 (m, 1H); 7.43 (m, 1H); 7.53 (d, 2H, J=8.2 Hz).

### Example 12.21.

### 2-butyl-1-[(4′-((1-ethoxycarbonyl-1,1-4-yl)methyl]-4-spirocyclohexyl-1H-imidazol-5(4H)-one

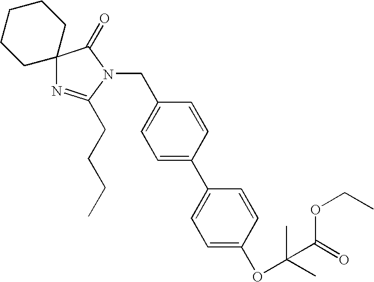

Prepared following the general procedure previously described (Method 12A) using 2-butyl-4-spirocyclohexyl-1H-imidazol-5(4H)-one (example 3.5) and ethyl 2-((4′-bromomethylbiphenyl-4-yl)oxy)-2-methylpropanoate (example 6.3). The product was chromatographed over silica gel (eluent cyclohexane/ethyl acetate 9/1 to 6/4). The product was obtained as a colorless oil.

Yield: 28%

Rf (dichloromethane/methanol 98/2): 0.25

IR: νCO: 1726 cm⁻¹; 1635 cm⁻¹

NMR ¹H (CDCl₃): 0.85 (t, 3H, J=7.3 Hz); 1.25 (t, 3H, J=7 Hz); 1.33 (sext, 2H, J=7.6 Hz); 1.46-1.78 (m, 12H); 1.62 (s, 6H); 2.33 (t, 2H, J=7.3 Hz); 4.24 (q, 2H, J=7 Hz); 4.68 (s, 2H); 6.89 (d, 2H, J=8.5 Hz); 7.18 (d, 2H, J=8.2 Hz); 7.43 (d, 2H, J=8.5 Hz); 7.49 (d, 2H, J=7.9 Hz).

### Example 12.22.

### 1-[(5′-bromo-2′-((1-ethoxycarbonyl-1,1-dimethylmethyl)-oxy)biphenyl-4-yl)methyl]-2-butyl-4-phenyl-1H-imidazol-5(4H)-one and 1-[(-2′-((1-ethoxycarbonyl-1,1-dimethylmethyl)oxy)biphenyl-4-yl)methyl]-2-butyl-4-phenyl-1H-imidazol-5(4H)

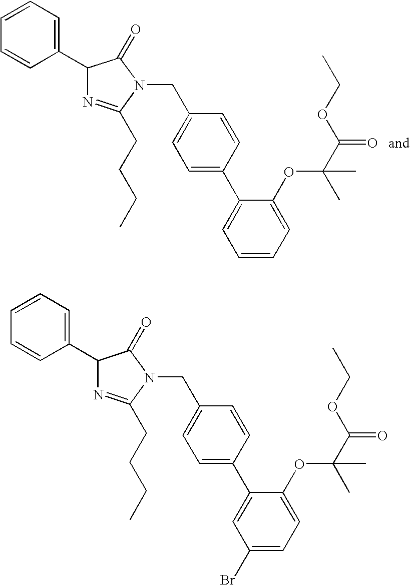

The compounds were prepared following the general procedure previously described (Method 12A) using 2-butyl-4-phenyl-1H-imidazol-5(4H)-one (example 3.7) and a mixture of ethyl 2-((4′-bromomethylbiphenyl-2-yl)oxy)-2-methylpropanoate and ethyl 2-((5-bromo-4′-bromomethyl-biphenyl-2-yl)oxy)-2-methylpropanoate (example 6.1). The products were chromatographed over silica gel (eluent dichloromethane/methanol 95/5). The products were obtained as a yellowish oil (mixture of 2 compounds).

Total yield: 48%

Rf (dichloromethane/methanol 98/2): 0.25

IR: νCO: 1732 cm⁻¹; 1645 cm⁻¹

NMR ¹H (CDCl₃) (derivative non-brominated on the aromatic cycle): 0.86 (t, 3H, J=7.3 Hz); 1.20-1.42 (m, 11H); 1.42-1.60 (quint, 2H, J=7.3 Hz); 2.31 (t, 2H, J=7.6 Hz); 3.3-3.5 (m, 2H); 4.24 (q, 2H, J=7.3 Hz); 5.32 (s, 1 H); 6.90 (d, 1H, J=8.2 Hz); 7.08 (t, 1H, J=7.3 Hz); 7.17-7.45 (m, 9H); 7.76 (d, 2H, J=8.2 Hz).

NMR ¹H (CDCl₃) (derivative brominated on the aromatic cycle): 0.98 (t, 3H, J=7.3 Hz); 1.20-1.42 (m, 11H); 1.80-1.95 (quint, 2H, J=7.3 Hz); 2.79 (t, 2H, J=7.6 Hz); 3.3-3.5 (m, 2H); 4.22 (q, 2H, J=7.3 Hz); 5.32 (s, 1H); 6.79 (d, 1h, J=8.2 Hz); 7.17-7.45 (m, 9H); 7.82 (d, 2H, J=8.2 Hz).

### Example 12.23.

### 1-[(3′-((1-tert-butyloxycarbonyl-1,1-dimethylmethyl)oxy)biphenyl-4-yl)methyl]-2-(2-methyl)propyl-4-spirocyclopentyl-1H-imidazol-5(4H)-one

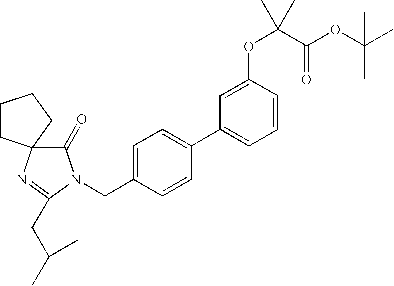

Prepared following the general procedure previously described (Method 12B) using 2-isobutyl-4-spirocyclopentyl-1H-imidazol-5(4H)-one (example 3.8) and tert-butyl 2-((4′-bromomethylbiphenyl-3-yl)oxy)-2-methylpropanoate (example 6.4). The product was chromatographed over silica gel (elution gradient cyclohexane/ethyl acetate 9/1 to 7/3). The product was obtained as a yellow oil.

Yield: 47%.

NMR ¹H (CDCl₃): 0.96 (d, 6H, J=6.7 Hz); 1.45 (s, 9H); 1.71 (s, 6H); 1.90-2.20 (m, 10H); 2.50 (m, 1H); 4.89 (s, 2H); 7.02 (d, 1H, J=7.3 Hz); 7.21 (s, 1H); 7.25 (d, 2H, J=8.2 Hz); 7.31 (m, 1H); 7.42 (t, 1H, J=7.9 Hz); 7.60 (d, 2H, J=8.2 Hz).

HPLC: purity: 98%.

### Example 12.24.

### 2-benzyl-1-[(3′-((1-tert-butyloxycarbonyl-1,1-dimethylmethyl)oxy)biphenyl-4-yl)methyl]-4-spirocyclopentyl-1H-imidazol-5(4H)-one

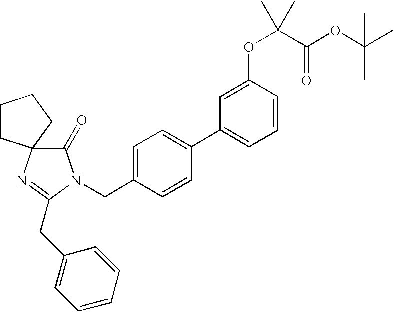

Prepared following the general procedure previously described (Method 12B) using 2-benzyl-4-spirocyclopentyl-1H-imidazol-5(4H)-one (example 3.9) and tert-butyl 2-((4′-bromomethylbiphenyl-3-yl)oxy)-2-methylpropanoate (example 6.4). The product was chromatographed over silica gel (eluent cyclohexane/ethyl acetate 9/1). The product was obtained as a yellow oil.

Yield: 7%

NMR ¹H (CDCl₃): 1.45 (s, 9H); 1.65 (s, 6H); 1.90-2.19 (m, 8H); 3.72 (s, 2H); 4.49 (s, 2H); 6.88 (dd, 1H, J=8.2 Hz, J=2 Hz); 7.12 (m, 1H); 7.21 (m, 2H); 7.28-7.40 (m, 2H); 7.52 (d, 2H, J=8 Hz).

### Example 12.25.

### 1-[(3′-((1-tert-butyloxycarbonyl-1,1-dimethylmethyl)oxy)biphenyl-4-yl)methyl]-2-cyclopropyl-4-spirocyclopentyl-1H-imidazol-5(4H)-one

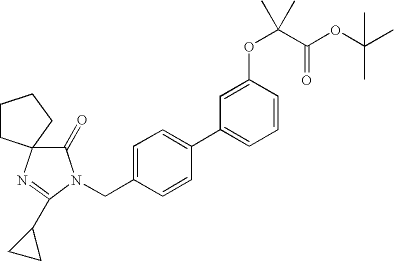

Prepared following the general procedure previously described (Method 12B) using 2-cyclopropyl-4-spirocyclopentyl-1H-imidazol-5(4H)-one (example 3.10) and tert-butyl 2-((4′-bromomethylbiphenyl-3-yl)oxy)-2-methylpropanoate (example 6.4). The product was chromatographed over silica gel (eluent cyclohexane/ethyl acetate 95/5). The product was obtained as an oil.

Yield: 30%

NMR ¹H (CDCl₃): 0.85 (m, 2H); 0.88 (m, 2H); 1.45 (s, 9H); 1.52 (m, 1H); 1.61 (s, 6H); 1.78 (m, 2H); 1.88-2.05 (m, 6H); 4.85 (s, 2H); 6.85 (dd, 1H, J=8.2 Hz, J=2Hz); 7.11 (s, 1H); 7.19 (d, 1H, J=7.9 Hz); 7.22-7.33 (m, 3H); 7.53 (d, 2H, J=8Hz).

### Example 12.26.

### 1-[(3′-((1-tert-butyloxycarbonyl-1,1-dimethylmethyl)oxy)biphenyl-4-yl)methyl]-2-(thiophen-2-yl)methyl-4-spirocyclopentyl-1H-imidazol-5(4H)-one

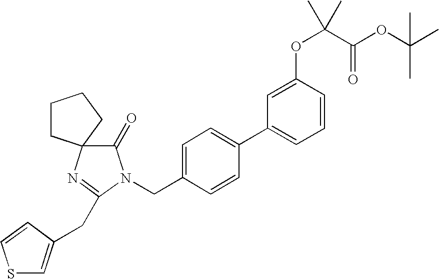

Prepared following the general procedure previously described (Method 12B) using 2-(thiophen-2-yl)-4-spirocyclopentyl-1H-imidazol-5(4H)-one (example 3.11) and tert-butyl 2-((4′-bromomethylbiphenyl-3-yl)oxy)-2-methylpropanoate (example 6.4). The product was chromatographed over silica gel (eluent cyclohexane/ethyl acetate 5/9, then 95/5, then 9/1). The product was obtained as an oil.

Yield: 20%

NMR ¹H (CDCl₃): 1.50 (s, 9H); 1.68 (s, 6H); 1.89-2.20 (m, 8H); 3.75 (s, 2H); 4,59 (s, 2H); 6.90 (d, 1H, J=8.2 Hz); 6.99 (d, 1H, J=4 Hz); 7.08 (s, 1H); 7.12-7.19 (m, 3H); 7.22 (d, 1H, J=7 Hz); 7.31-7.40 (m, 2H); 7.55 (d, 2H, J=8 Hz).

### Example 12.27.

### 2-butyl-1-[[4-[(4-((1-ethoxycarbonyl-1,1-dimethylmethyloxy)phenyl)methyl]phenyl]methyl]-4-spirocyclopentyl-1H-imidazol-5(4H)-one

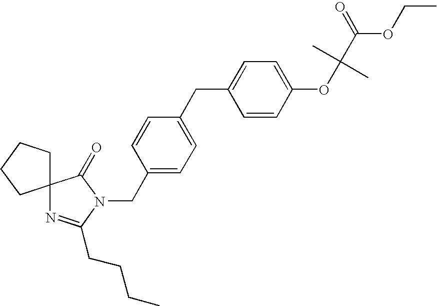

Prepared following the general procedure previously described (Method 12B) using 2-butyl-4-spirocyclopentyl-1H-imidazol-5(4H)-one (example 3.4) and ethyl 2-[4-[4-bromomethylbenzyl)phenyloxy]-2-methylpropanoate (example 8.3). The product was chromatographed over silica gel (elution gradient cyclohexane/ethyl acetate 9/1 to 7/3, then 9/1). The product was obtained as a white oil.

Yield: 30%

Rf (cyclohexane/ethyl acetate 7/3): 0.25

NMR ¹H (CDCl₃): 0.84 (t, 3H, J=7.2 Hz); 1.24 (t, 3H, J=7 Hz); 1.40-1.55 (m, 6H); 1.57 (s, 6H); 1.50-2.10 (m, 6H); 2.31 (t, 2H, J=8 Hz); 3.88 (s, 2H); 4.24 (q, 2H, J=7 Hz); 4.64 (s, 2H); 6.75 (d, 2H, J=8 Hz); 6.94-7.12 (m, 6H).

### Example 12.28.

### 2-butyl-1-[(4′-((4-methyloxycarbonyl-4,4-dimethylbutan-1-yl)oxy)biphenyl-4-yl)methyl]-4-spirocyclopentyl-1H-imidazol-5(4H)-one

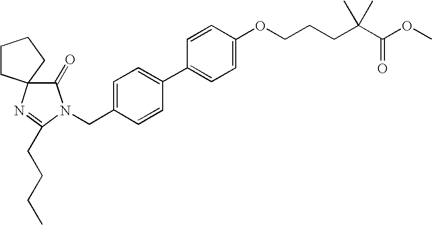

Prepared following the general procedure previously described (Method 12B) using 2-butyl-4-spirocyclopentyl-1H-imidazol-5(4H)-one (example 3.4) and methyl 5-(4′-bromomethyl-biphenyl-4-yloxy)-2,2-dimethyl-pentanoate (example 6.6). The product was chromatographed over silica gel (eluent cyclohexane/ethyl acetate 7/3, and then an elution gradient cyclohexane/ethyl acetate 8/2 to 7/3). The product was obtained as a colorless oil.

Yield: 45%

Rf (ethyl acetate/cyclohexane 6/4): 0.30

NMR ¹H (CDCl₃): 0.88 (t, 3H, J=7.2 Hz); 1.24 (s, 6H); 1.27 (m, 2H); 1.36 (sext, 2H, J=7.6 Hz); 1.59 (quint, 2H, J=8.4 Hz); 1.75 (m, 2H); 1.90-2.10 (m, 8H); 2.34 (t, 2H, J=8 Hz); 3.68 (s, 3H); 4.00 (t, 2H, J=6 Hz); 4.72 (s, 2H); 6.96 (d, 2H, J=9.2 Hz); 7.21 (d, 2H, J=8 Hz); 7.50 (d, 2H, J=8.8 Hz); 7.52 (d, 2H, J=8.4 Hz).

### Example 12.29.

### 2-butyl-1-[(3′-((4-methoxycarbonyl-4,4-dimethylbutan-1-yl)oxy)biphenyl -4-yl) methyl]-4-spirocyclopentyl-1H-imidazol-5(4H)-one

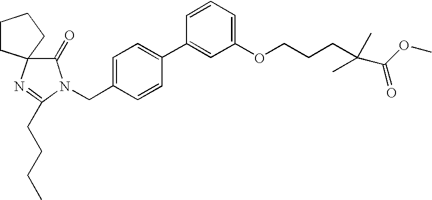

Prepared following the general procedure previously described (Method 12B) using 2-butyl-4-spirocyclopentyl-1H-imidazol-5(4H)-one (example 3.4) and methyl 5-(4′-bromomethyl-biphenyl-3-yloxy)-2,2-dimethyl-pentanoate (example 6.7). The product was chromatographed over silica gel (eluent cyclohexane/ethyl acetate 7/3). The product was obtained as a yellow oil.

Yield: 40%

Rf (cyclohexane/ethyl acetate 7/3): 0.25

NMR ¹H (CDCl₃): 0.85 (t, 3H, J=7.2 Hz); 1.12 (s, 6H); 1.20-1.40 (m, 2H); 1.45-1.68 (m, 8H); 1.75-2.05 (m, 6H); 2.32 (t, 2H, J=8 Hz); 3.60 (s, 3H); 3.90 (t, 2H, J=6 Hz); 4.69 (s, 2H); 6.96 (m, 2H); 7.15 (d, 2H, J=8 Hz); 7.20-7.30 (m, 2H); 7.50 (d, 2H, J=8.4 Hz).

### Example 12.30.

### 2-butyl-1-[[4-[(4-(1-tert-butyloxycarbonyl-1,1-dimethylmethyloxy)phenyl)oxy]phenyl]methyl]-4-spirocyclopentyl-1H-imidazol-5(4H)-one

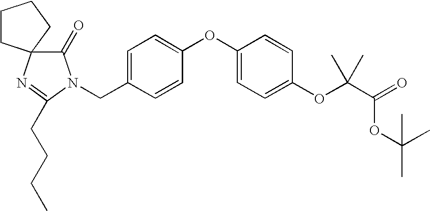

Prepared following the general procedure previously described (Method 12B) using 2-butyl-4-spirocyclopentyl-1H-imidazol-5(4H)-one (example 3.4) and tert-butyl 2-[4-(4-bromomethylphenyloxy)phenyloxy]-2-methylpropanoate (example 9.1). The product was chromatographed over silica gel (eluent cyclohexane/ethyl acetate 7/3). The product was obtained as a viscous white oil.

Yield: 11%

Rf (cyclohexane/ethyl acetate 7/3): 0.30

NMR ¹H (CDCl₃): 0.87 (t, 3H, J=7.2 Hz); 1.33 (sext, 2H, J=7 Hz); 1.48 (s, 9H); 1.55 (s, 6H); 1.50-1.65 (m, 2H); 1.80 (m, 2H); 1.87-2.08 (m, 6H); 2.32 (t, 2H, J=8 Hz); 4.65 (s, 2H); 6.85-7.00 (m, 6H); 7.10 (d, 2H, J=8 Hz).

### Example 12.31.

### 2-butyl-1-[[4-[(4-(1-tert-butyloxycarbonyl-1,1-dimethylmethyloxy)phenyl)oxy]phenyl]methyl]-4-spirocyclohexyl-1H-imidazol-5(4H)-one

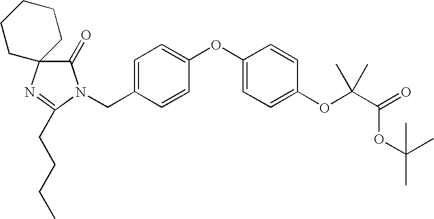

Prepared following the general procedure previously described (Method 12B) using 2-butyl-4-spirocyclohexyl-1H-imidazol-5(4H)-one (example 3.5) and tert-butyl 2-[4-(4-bromomethylphenyloxy)phenyloxy]-2-methylpropanoate (example 9.1). The product was chromatographed over silica gel (eluent cyclohexane/ethyl acetate 8/2). The product was obtained as a colorless viscous oil.

Yield: 18%

Rf (cyclohexane/ethyl acetate 7/3): 0.30

NMR ¹H (CDCl₃): 0.89 (t, 3H, J=7.2 Hz); 1.34 (sext, 2H, J=7 Hz); 1.40-1.50 (m, 2H); 1.48 (s, 9H); 1.55 (s, 6H); 1.50-1.90 (m, 10H); 2.32 (t, 2H, J=8 Hz); 4.62 (s, 2H); 6.80-6.98 (m, 6H); 7.10 (d, 2H, J=8 Hz).

### Example 12.32.

### 2-butyl-1-[[4-[(3-((1-ethoxycarbonyl-1,1-dimethylmethyloxy)phenyl)methyl]phenyl]methyl]-4-spirocyclopentyl-1H-imidazol-5(4H)-one

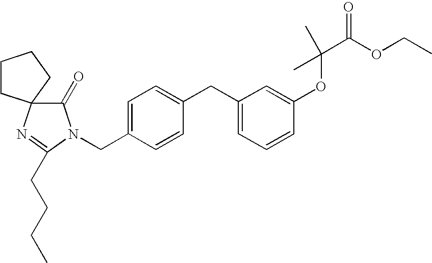

Prepared following the general procedure previously described (Method 12B) using 2-butyl-4-spirocyclopentyl-1H-imidazol-5(4H)-one (example 3.4) and ethyl 2-[3-[4-bromomethylbenzyl)phenyloxy]-2-methylpropanoate (example 8.1). The product was chromatographed over silica gel (eluent cyclohexane/ethyl acetate 8/2, then 7/3 and 6/4). The product was obtained as an oil.

Yield: 20%

Rf (cyclohexane/ethyl acetate 7/3): 0.25

NMR ¹H (CDCl₃): 0.85 (t, 3H, J=7.2 Hz); 1.20 (t, 3H, J=7 Hz); 1.25 (sext, 2H, J=7 Hz); 1.45-1.70 (m, 2H); 1.55 (s, 6H); 1.72-2.10 (m, 8H); 2.29 (t, 2H, J=8 Hz); 3.90 (s, 2H); 4.17 (q, 2H, J=7 Hz); 4.65 (s, 2H); 6.60-6.70 (m, 2H); 6.79 (d, 1H, J=8 Hz); 7.02-7.20 (m, 5H).

### Example 12.33.

### 2-butyl-1-[[4-[(3-((1-ethoxycarbonyl-1,1-dimethylmethyloxy)phenyl)methyl]phenyl]methyl]-4-spirocyclohexyl-1H-imidazol-5(4H)-one

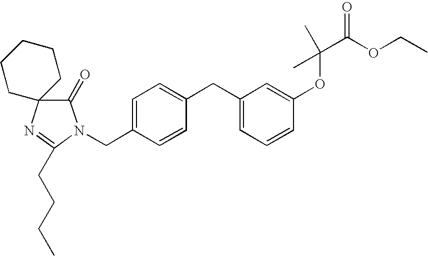

Prepared following the general procedure previously described (Method 12B) using 2-butyl-4-spirocyclohexyl-1H-imidazol-5(4H)-one (example 3.5) and ethyl 2-[3-[4-bromomethylbenzyl)phenyloxy]-2-methylpropanoate (example 8.1). The product was chromatographed over silica gel (elution gradient cyclohexane/ethyl acetate 8/2 to 6/4, then 9/1). The product was obtained as an oil.

Yield: 16%

Rf (cyclohexane/ethyl acetate 7/3): 0.25

NMR ¹H (CDCl₃): 0.86 (t, 3H, J=7.2 Hz); 1.20 (t, 3H, J=7 Hz); 1.28 (sext, 2H, J=7 Hz); 1.48-1.60 (m, 2H); 1.55 (s, 6H); 1.60-2.05 (m, 10H); 2.30 (t, 2H, J=8 Hz); 3.89 (s, 2H); 4.18 (q, 2H, J=7 Hz); 4.62 (s, 2H); 6.60-6.70 (m, 2H); 6.79 (d, 1H, J=8 Hz); 7.00-7.20 (m, 5H).

### Example 12.34.

### 2-butyl-1-[(4′-((4-methoxycarbonyl-4,4-dimethylbutan-1-yl)oxy)biphenyl-4-yl)methyl]-4-spirocyclohexyl-1H-imidazol-5(4H)-one

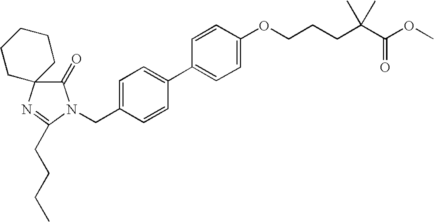

Prepared following the general procedure previously described (Method 12B) using 2-butyl-4-spirocyclohexyl-1H-imidazol-5(4H)-one (example 3.5) and methyl 5-(4′-bromomethyl-biphenyl-4-yloxy)-2,2-dimethyl-pentanoate (example 6.6). The product was chromatographed over silica gel (eluent cyclohexane/ethyl acetate 98/2, then 9/1 and 8/2). The product was obtained as a yellow oil.

Yield: 16%

Rf (ethyl acetate/cyclohexane 6/4): 0.30

NMR ¹H (CDCl₃): 0.87 (t, 3H, J=7.2 Hz); 1.22 (s, 6H); 1.27 (t, 2H, J=7.2 Hz); 1.35 (sext, 2H, J=7.6 Hz); 1.45-1.63 (m, 4H); 1.65-1.88 (m, 10H); 2.36 (t, 2H, J=8 Hz); 3.67 (s, 3H); 3.97 (t, 2H, J=6 Hz); 4.69 (s, 2H); 6.94 (d, 2H, J=9.2 Hz); 7.18 (d, 2H, J=8 Hz); 7.47 (d, 2H, J=8.8 Hz); 7.50 (d, 2H, J=8.4 Hz).

### Example 12.35.

### 2-butyl-1-[[4-[(3-(1-tert-butyloxycarbonyl-1,1-dimethylmethyloxy)phenyl)oxy]phenyl]methyl]-4-spirocyclohexyl-1H-imidazol-5(4H)-one

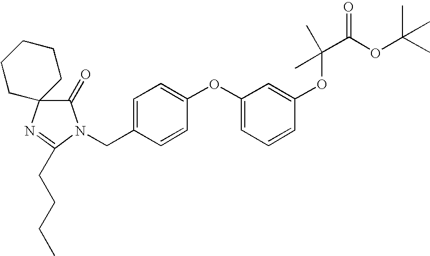

Prepared following the general procedure previously described (Method 12B) using 2-butyl-4-spirocyclohexyl-1H-imidazol-5(4H)-one (example 3.5) and tert-butyl 2-[3-(4-bromomethylphenyloxy)phenyloxy]-2-methylpropanoate (example 9.2). The product was chromatographed over silica gel (eluent cyclohexane/ethyl acetate 9/1). The product was obtained as a colorless oil.

Yield: 19%

Rf (cyclohexane/ethyl acetate 7/3): 0.30

NMR ¹H (CDCl₃): 0.89 (t, 3H, J=7.2 Hz); 1.30-1.85 (m, 14H); 1.40 (s, 9H) 1.55 (s, 6H); 2.32 (t, 2H, J=8 Hz); 4.65 (s, 2H); 6.51 (s, 1H); 6.60 (d, 2H, J=8Hz); 6.95 (d, 2H, J=8 Hz); 7.10 (d, 2H, J=8 Hz); 7.17 (t, 1H, J=8 Hz).

### Example 12.36.

### 2-butyl-1-[[4-[(3-(1-tert-butyloxycarbonyl-1,1-dimethylmethyloxy)phenyl)oxy]phenyl]methyl]-4-spirocyclopentyl-1H-imidazol-5(4H)-one

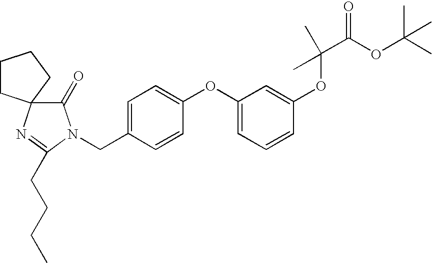

Prepared following the general procedure previously described (Method 12B) using 2-butyl-4-spirocyclopentyl-1H-imidazol-5(4H)-one (example 3.4) and tert-butyl 2-[3-(4-bromomethylphenyloxy)phenyloxy]-2-methylpropanoate (example 9.2). The product was chromatographed over silica gel (eluent cyclohexane/ethyl acetate 9/1). The product was obtained as a colorless oil.

Yield: 51%

Rf (cyclohexane/ethyl acetate 7/3): 0.30

NMR ¹H (CDCl₃): 0.88 (t, 3H, J=7.2 Hz); 1.32 (sext, 2H, J=7 Hz); 1.42 (s, 9H); 1.50-1.68 (m, 2H); 1.55 (s, 6H); 1.70-2.08 (m, 8H); 2.32 (t, 2H, J=8 Hz); 4.65 (s, 2H); 6.52 (d, 1H, J=2 Hz); 6.61 (d, 2H, J=8Hz); 6.98 (d, 2H, J=8 Hz); 7.13 (d, 2H, J=8 Hz); 7.15 (t, 1H, J=8 Hz).

### Example 12.37.

### 2-butyl-1-[[4-[(2-((1-ethoxycarbonyl-1,1-dimethylmethyloxy)phenyl)methyl]phenyl]methyl]-4-spirocyclopentyl-1H-imidazol-5(4H)-one

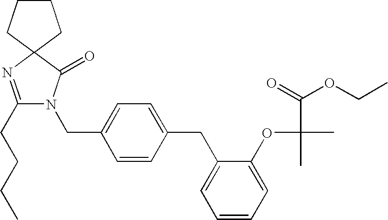

Prepared following the general procedure previously described (Method 12B) using 2-butyl-4-spirocyclopentyl-1H-imidazol-5(4H)-one (example 3.4) and ethyl 2-[2-[4-bromomethylbenzyl)phenyloxy]-2-methylpropanoate (example 8.2). The product was chromatographed over silica gel (elution gradient cyclohexane/ethyl acetate 10/0 to 7/3). The product was obtained as a colorless oil.

Yield: 34%

Rf (ethyl acetate/cyclohexane 6/4): 0.45

NMR ¹H (CDCl₃): 0.85 (t, 3H, J=7 Hz); 1.23 (t, 3H, J=7 Hz); 1.11-1.38 (m, 2H); 1.40-1.52 (m, 2H); 1.45 (s, 6H); 1.70-2.10 (m, 8H); 2.29 (t, 2H, J=8 Hz); 3.96 (s, 2H); 4.21 (q, 2H, J=7 Hz); 4.64 (s, 2H); 6.60 (dd, 1H, J=9.2 Hz, J=2Hz); 6.88 (t, 1H, J=8 Hz); 7.00-7.30 (m, 6H).

### Example 12.38.

### 2-butyl-1-[(2′-((4-methoxycarbonyl-4,4-dimethylbutan-1-yl)oxy)biphenyl-4-yl)methyl]-4-spirocyclohexyl-1H-imidazol-5(4H)-one

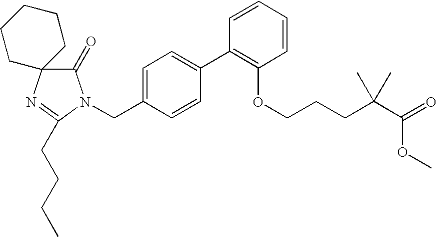

Prepared following the general procedure previously described (Method 12B) using 2-butyl-4-spirocyclohexyl-1H-imidazol-5(4H)-one (example 3.5) and methyl 5-(4′-bromomethyl-biphenyl-2-yloxy)-2,2-dimethyl-pentanoate (example 6.8). The product was chromatographed over silica gel (eluent cyclohexane/ethyl acetate 98/2, then 9/1 and 8/2). The product was obtained as an oil.

Yield: 12%

Rf (cyclohexane/ethyl acetate 7/3): 0.28

NMR ¹H (CDCl₃): 0.87 (t, 3H, J=7 Hz); 1.14 (s, 6H); 1.33 (sext, 2H, J=7.2 Hz); 1.45-1.90 (m, 16H); 2.36 (t, 2H, J=8 Hz); 3.62 (s, 3H); 3.93 (t, 2H, J=6 Hz); 4.71 (s, 2H); 6.93 (d, 1H, J=8 Hz); 7.02 (t, 1H, J=8 Hz); 7.17 (d, 2H, J=8 Hz); 7.29 (d, 8 Hz); 7.52 (d, 2H, J=8 Hz).

### Example 12.39.

### 2-butyl-1-[(2′-((7-methoxycarbonyl-7,7-dimethylheptan-1-yl)oxy)biphenyl-4-yl)methyl]-4-spirocyclopentyl-1H-imidazol-5(4H)-one

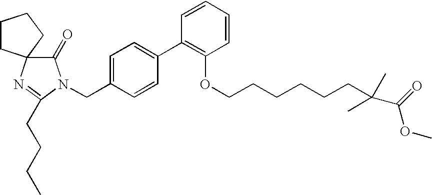

Prepared following the general procedure previously described (Method 12B) using 2-butyl-4-spirocyclopentyl-1H-imidazol-5(4H)-one (example 3.4) and methyl 8-(4′-bromomethyl-biphenyl-2-yloxy)-2,2-dimethyl-octanoate (example 6.9). The product was chromatographed over silica gel (eluent cyclohexane/ethyl acetate 9/1). The product was obtained as a yellowish oil.

Yield: 60%

Rf (cyclohexane/ethyl acetate 7/3): 0.26

NMR ¹H (CDCl₃): 0.87 (t, 3H, J=7 Hz); 1.16 (s, 6H); 1.17-1.43 (m, 8H); 1.50 (m, 2H); 1.60 (m, 2H); 1.71 (m, 2H); 1.82 (m, 2H); 1.90-2.10 (m, 6H); 2.35 (t, 2H, J=8 Hz); 3.63 (s, 3H); 3.95 (t, 2H, J=6 Hz); 4.70 (s, 2H); 6.93 (d, 1H, J=8 Hz); 7.01 (t, 1H, J=8 Hz); 7.15 (d, 2H, J=8 Hz); 7.30 (d, 2H, J=8 Hz); 7.51 (d, 2H, J=8 Hz).

### Example 12.40.

### 2-butyl-1-[(2′-((4-methoxycarbonyl-4,4-dimethylbutan-1-yl)oxy)biphenyl-4-yl)methyl]-4-spirocyclopentyl-1H-imidazol-5(4H)-one

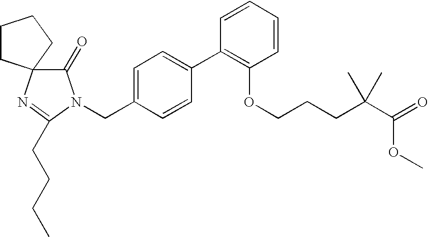

Prepared following the general procedure previously described (Method 12B) using 2-butyl-4-spirocyclopentyl-1H-imidazol-5(4H)-one (example 3.4) and methyl 5-(4′-bromomethyl-biphenyl-2-yloxy)-2,2-dimethyl-pentanoate (example 6.8). The product was chromatographed over silica gel (eluent cyclohexane/ethyl acetate 98/2, then 9/1 and 8/2). The product was obtained as an oil.

Yield: 41%

Rf (cyclohexane/ethyl acetate 7/3): 0.26

NMR ¹H (CDCl₃): 0.87 (t, 3H, J=7 Hz); 1.14 (s, 6H); 1.33 (sext, 2H, J=7.2 Hz); 1.52-1.73 (m, 8H); 1.90-2.10 (m, 6H); 2.35 (t, 2H, J=8 Hz); 3.63 (s, 3H); 3.93 (t, 2H, J=6 Hz); 4.72 (s, 2H); 6.93 (d, 1H, J=8 Hz); 7.01 (t, 1H, J=8 Hz); 7.18 (d, 2H, J=8 Hz); 7.29 (d, 2H, J=8 Hz); 7.52 (d, 2H, J=8 Hz).

### Example 12.41.

### 2-butyl-1-[[4-[(2-((1-ethoxycarbonyl-1,1-dimethylmethyloxy)phenyl)methyl]phenyl]methyl]-4-spirocyclohexyl-1H-imidazol-5(4H)-one

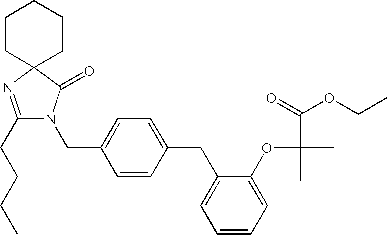

Prepared following the general procedure previously described (Method 12B) using 2-butyl-4-spirocyclohexyl-1H-imidazol-5(4H)-one (example 3.5) and ethyl 2-[2-[4-bromomethylbenzyl)phenyloxy]-2-methylpropanoate (example 8.2). The product was chromatographed over silica gel (eluent cyclohexane/ethyl acetate 7/3). The product was obtained as a colorless oil.

Yield: 16%

Rf (ethyl acetate/cyclohexane 6/4): 0.45

NMR ¹H (CDCl₃): 0.85 (t, 3H, J=7 Hz); 1.21 (t, 3H, J=7 Hz); 1.21-1.40 (m, 1.40-1.60 (m, 4H); 1.42 (s, 6H); 1.61-1.88 (m, 8H); 2.30 (t, 2H, J=8 Hz); 3.95 (s, 2H); 4.21 (q, 2H, J=7 Hz); 4.62 (s, 2H); 6.61 (d, 1H, J=9 Hz); 6.89 (t, 1H, J=8 Hz); 6.69-7.22 (m, 6H).

### Example 12.42.

### 2-butyl-1-[[4-[(3-(1-tert-butyloxycarbonyl-1,1-dimethylmethyloxy)phenyl)thio]phenyl]methyl]-4-spirocyclopentyl-1H-imidazol-5(4H)-one

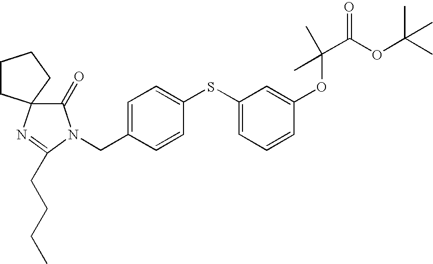

Prepared following the general procedure previously described (Method 12B) using 2-butyl-4-spirocyclopentyl-1H-imidazol-5(4H)-one (example 3.4) and tert-butyl 2-[3-(4-bromomethylphenylthio)phenyloxy]-2-methylpropanoate (example 9.4). The product was chromatographed over silica gel (eluent cyclohexane/ethyl acetate 9/1). The product was obtained as a colorless oil.

Yield: 35%

Rf (cyclohexane/ethyl acetate 7/3): 0.30

NMR ¹H (CDCl₃): 0.85 (t, 3H, J=7.2 Hz); 1.20-1.35 (m, 2H); 1.40 (s, 9H); 1.50-1.80 (m, 2H); 1.53 (s, 6H); 1.85-2.10 (m, 8H); 2.30 (t, 2H, J=8 Hz); 4.64 (s, 2H); 6.73 (dd, 1H, J=8 Hz, J=2 Hz); 6.84 (t, 1H, J=2 Hz); 6.92 (d, 1H, J=8 Hz); 7.10 (d, 2H, J=8 Hz); 7.19 (t, 1H, J=8 Hz); 7.28 (d, 2H, J=8 Hz).

### Example 12.43.

### 2-butyl-1-[[4-[(3-(1-tert-butyloxycarbonyl-1,1-dimethylmethyloxy)phenyl)thio]phenyl]methyl]-4-spirocyclohexyl-1H-imidazol-5(4H)-one

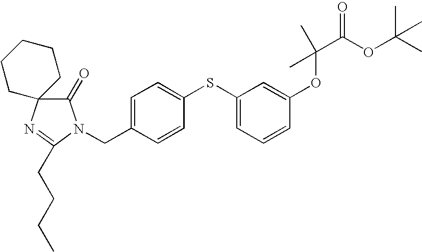

Prepared following the general procedure previously described (Method 12B) using 2-butyl-4-spirocyclohexyl-1H-imidazol-5(4H)-one (example 3.5) and tert-butyl 2-[3-(4-bromomethylphenylthio)phenyloxy]-2-methylpropanoate (example 9.4). The product was chromatographed over silica gel (eluent cyclohexane/ethyl acetate 9/1). The product was obtained as a yellow oil.

Yield: 21%

Rf (cyclohexane/ethyl acetate 7/3): 0.30

NMR ¹H (CDCl₃): 0.88 (t, 3H, J=7.2 Hz); 1.10-1.45 (m, 2H); 1.39 (s, 9H); 1.52 (s, 6H); 1.55-1.90 (m, 12H); 2.31 (t, 2H, J=8 Hz); 4.62 (s, 2H); 6.73 (dd, 1H, J=8 Hz, J=2 Hz); 6.82 (d, 1H, J=2 Hz); 6.93 (d, 1H, J=8 Hz); 7.08 (d, 2H, J=8 Hz); 7.19 (t, 1H, J=8 Hz); 7.28 (d, 2H, J=8 Hz).

### Example 12.44.

### 2-butyl-1-[[4-[(4-(1-tert-butyloxycarbonyl-1,1-dimethylmethyloxy)phenyl)thio]phenyl]methyl]-4-spirocyclohexyl-1H-imidazol-5(4H)-one

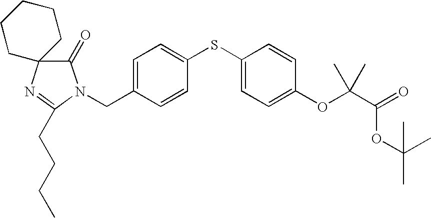

Prepared following the general procedure previously described (Method 12B) using 2-butyl-4-spirocyclohexyl-1H-imidazol-5(4H)-one (example 3.5) and tert-butyl 2-[4-(4-bromomethylphenylthio)phenyloxy]-2-methylpropanoate (example 9.3). The product was chromatographed over silica gel (eluent cyclohexane/ethyl acetate 95/5). The product was obtained as a yellow oil.

Yield: 12%

Rf (cyclohexane/ethyl acetate 7/3): 0.30

NMR ¹H (CDCl₃): 0.86 (t, 3H, J=7.2 Hz); 1.20-1.38 (m, 2H); 1.42 (s, 9H); 1.60 (s, 6H); 1.50-1.85 (m, 12H); 2.29 (t, 2H, J=8 Hz); 4.60 (s, 2H); 6.82 (d, 2H, J=8 Hz); 7.00 (d, 2H, J=2 Hz); 7.10 (d, 2H, J=8 Hz); 7.30 (d, 2H, J=8 Hz).

### Example 12.45.

### 2-butyl-1-[(3′-((2-methoxycarbonyl-2,2-dimethylethyl-1-yl)oxy)biphenyl-4-yl)methyl]-4-spirocyclopentyl-1H-imidazol-5(4H)-one

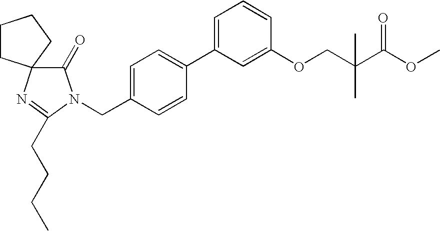

Prepared following the general procedure previously described (Method 12B) using 2-butyl-4-spirocyclopentyl-1H-imidazol-5(4H)-one (example 3.4) and methyl 3-(4′-bromomethyl-biphenyl-3-yloxy)-2,2-dimethyl-propanoate (example 6.10). The product was chromatographed over silica gel (eluent cyclohexane/ethyl acetate 98/2, then 9/1 and 8/2). The product was obtained as a yellow oil.

Yield: 34%

Rf (cyclohexane/ethyl acetate 7/3): 0.25

NMR ¹H (CDCl₃): 0.87 (t, 3H, J=7.2 Hz); 1.18 (s, 6H); 1.32 (sext, 2H, J=7 Hz); 1.48-1.70 (m, 2H); 1.75-2.10 (m, 8H); 2.33 (t, 2H, J=8 Hz); 3.57 (s, 3H); 3.95 (s, 2H); 4.72 (s, 2H); 7.00 (m, 2H); 7.14 (d, 2H, J=8 Hz); 7.21-7.35 (m, 4H); 7.44 (d, 2H, J=8.4 Hz).

### Example 12.46.

### 2-butyl-1-[[4-[(4-((1-ethoxycarbonyl-1,1-dimethylmethyloxy)phenyl)methyl]phenyl]methyl]-4-spirocyclohexyl-1H-imidazol-5(4H)-one

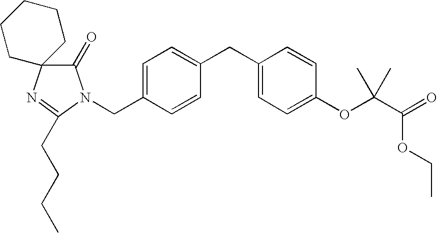

Prepared following the general procedure previously described (Method 12B) using 2-butyl-4-spirocyclohexyl-1H-imidazol-5(4H)-one (example 3.5) and ethyl 2-[4-[4-bromomethylbenzyl)phenyloxy]-2-methylpropanoate (example 8.3). The product was chromatographed over silica gel (eluent cyclohexane/ethyl acetate 9/1, then 8/2) The product was obtained as a colorless oil.

Yield: 60%

Rf (cyclohexane/ethyl acetate 7/3): 0.25

NMR ¹H (CDCl₃): 0.84 (t, 3H, J=7.2 Hz); 1.24 (t, 3H, J=7 Hz); 1.30-1.59 (m, 6H); 1.57 (s, 6H); 1.60-1.90 (m, 8H); 2.30 (t, 2H, J=8 Hz); 3.88 (s, 2H); 4.22 (q, 2H, J=7 Hz); 4.62 (s, 2H); 6.75 (d, 2H, J=8 Hz); 6.90-7.15 (m, 6H).

### Example 12.47.

### 2-butyl-1-[[4-[(4-(1-tert-butyloxycarbonyl-1,1-dimethylmethyloxy)phenyl)thio]phenyl]methyl]-4-spirocyclopentyl-1H-imidazol-5(4H)-one

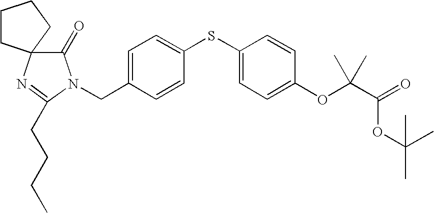

Prepared following the general procedure previously described (Method 12B) using 2-butyl-4-spirocyclopentyl-1H-imidazol-5(4H)-one (example 3.4) and tert-butyl 2-[4-(4-bromomethylphenylthio)phenyloxy]-2-methylpropanoate (example 9.3). The product was chromatographed over silica gel (eluent cyclohexane/ethyl acetate 9/1, then 8/2). The product was obtained as a colorless oil.

Yield: 21%

Rf (cyclohexane/ethyl acetate 7/3): 0.30

NMR ¹H (CDCl₃): 0.85 (t, 3H, J=7.2 Hz); 1.18-1.35 (m, 2H); 1.45 (s, 9H); 1.60 (s, 6H); 1.50-1.85 (m, 10H); 2.28 (t, 2H, J=8 Hz); 4.60 (s, 2H); 6.82 (d, 2H, J=8 Hz); 7.00 (d, 2H, J=2 Hz); 7.09 (d, 2H, J=8 Hz); 7.31 (d, 2H, J=8 Hz).

### Example 12.48.

### 2-butyl-1-[(4-bromophenyl)methyl]-4-spirocyclohexyl-1H-imidazol-5(4H)-one

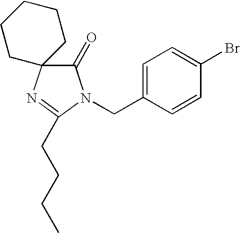

Prepared following the general procedure previously described (Method 12C) using 2-butyl-4-spirocyclohexyl-1H-imidazol-5(4H)-one (example 3.5) and 1-bromo-4-(bromomethyl)benzene. The product was obtained as a colorless oil.

Yield: 70%

Rf (cyclohexane/ethyl acetate 6/4): 0.60

NMR ¹H (CDCl₃): 0.88 (t, 3H, J=7.3 Hz); 1.35 (m, 2H); 1.54 (m, 4H); 1.74 (m, 8H); 2.31 (t, 2H, J=7.3 Hz); 4.62 (s, 2H); 7.04 (d, 2H, J=8.5 Hz); 7.47 (d, 2H, J=8.5 Hz).

### Example 12.49.

### 2-butyl-1-[(4-bromophenyl)methyl]-4,4-diethyl-1H-imidazol-5(4H)-one

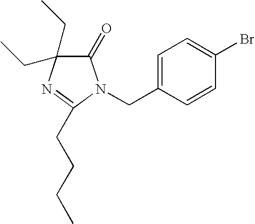

Prepared following the general procedure previously described (Method 12C) using 2-butyl-4,4-diethyl-1H-imidazol-5(4H)-one (example 3.12) and 1-bromo-4-(bromomethyl)benzene. The product was chromatographed over silica gel (eluant cyclohexane/ethyl acetate 80/20). The product was obtained as a yellow oil.

Yield: 40.2%

Rf (cyclohexane/ethyl acetate 6/4): 0.5

NMR ¹H (CDCl₃): 0.65 (t, 6H, J=7.3 Hz); 0.84 (t, 3H, J=7.6 Hz); 1.31 (m, 2H); 1.59 (m, 2H); 1.76 (q, 4H, J=7.3 Hz); 2.32 (t, 2H, J=7.3 Hz); 4.57 (s, 2H); 7.06 (d, 2H, J=8.5 Hz); 7.42 (d, 2H, J=8.2 Hz).

### Example 12.50.

### 2-butyl-1-[(4-bromo-3-methylphenyl)methyl]-4-spirocyclohexyl-1H-imidazol-5(4H)-one

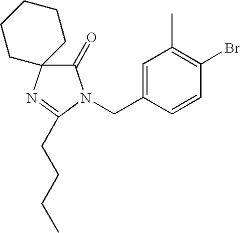

Prepared following the general procedure previously described (Method 12C) using 2-butyl-4-spirocyclohexyl-1H-imidazol-5(4H)-one (example 3.5) and 1-bromo-4-(bromomethyl)-2-methylbenzene. The product was chromatographed over silica gel (eluent cyclohexane/ethyl acetate 80/20). The product was obtained as a yellow oil.

Yield: 81.4%

Rf (cyclohexane/ethyl acetate 7/3): 0.5

NMR ¹H (CDCl₃): 0.87 (t, 3H, J=7.3 Hz); 1.33 (m, 2H); 1.44-1.79 (m, 12H); 2.3 (t, 2H, J=7.9 Hz); 2.37 (s, 3H); 4.59 (s, 2H); 6.82 (m, 1H); 7.01 (m, 1H); 7.47 (d, 1H, J=8.2 Hz).

### Example 12.51.

### 2-butyl-1-[(3-bromophenyl)methyl]-4-spirocyclopentyl-1H-imidazol-5(4H)-one

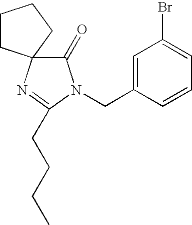

Prepared following the general procedure previously described (Method 12C) using 2-butyl-4-spirocyclopentyl-1H-imidazol-5(4H)-one (example 3.4) and 1-bromo-3-(bromomethyl)benzene. The product was chromatographed over silica gel (elution gradient petroleum ether/ethyl acetate 100/0 to 70/30). The product was obtained as a colorless oil.

Yield: 75%

Rf (dichloromethane/methanol 95/5): 0.13

NMR ¹H (CDCl₃): 0.87 (t, 3H, J=7.2 Hz); 1.32 (m, 2H); 1.56 (m, 2H); 1.80-2.02 (m, 8H); 2.29 (t, 2H, J=7.5 Hz); 4.65 (s, 2H); 7.09 (d, 1H, J=7.3 Hz); 7.21 (t, 1H, J=7.7 Hz); 7.30 (s, 1H); 7.42 (d, 1H, J=7.9 Hz).

### Example 12.52.

### 2-butyl-1-[(4-bromophenyl)methyl]-4-spirocyclopentyl-1H-imidazol-5(4H)-one

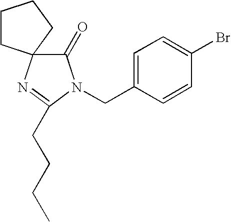

Prepared following the general procedure previously described (Method 12C) using 2-butyl-4-spirocyclopentyl-1H-imidazol-5(4H)-one (example 3.4) and 1-bromo-4-(bromomethyl)benzene. The product was chromatographed over silica gel (eluent cyclohexane/ethyl acetate 70/30). The product was obtained as a colorless oil.

Yield: 45.4%

Rf (cyclohexane/ethyl acetate 7/3): 0.25

NMR ¹H (CDCl₃): 0.86 (t, 3H, J=7.3 Hz); 1.31 (m, 2H); 1.55 (m, 2H); 1.80 (m, 2H); 1.96 (m, 6H); 2.27 (t, 2H, J=7.6 Hz); 4.61 (s, 2H); 7.02 (d, 2H, J=8.5 Hz); 7.45 (d, 2H, J=8.5 Hz).

### Example 12.53.

### 2-butyl-1-[(4-bromo-2-methoxyphenyl)methyl]-4-spirocyclopentyl-1H-imidazol-5(4H)-one

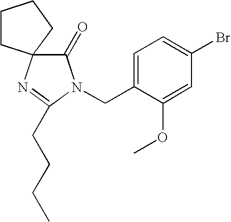

Prepared following the general procedure previously described (Method 12C) using 2-butyl-4-spirocyclopentyl-1H-imidazol-5(4H)-one (example 3.4) and 4-bromo-1-(bromomethyl)-2-methoxybenzene (example 10.5). The product was chromatographed over silica gel (elution gradient petroleum ether/ethyl acetate 100/0 to 70/30). The product was obtained as a colorless oil.

Yield: 69%

Rf (petroleum ether/ethyl acetate 60/40): 0.38

NMR ¹H (CDCl₃): 0.90 (t, 3H, J=7.5 Hz); 1.33-1.39 (m, 2H); 1.52-1.64 (m, 2H); 1.80-2.00 (m, 8H); 2.29-2.35 (m, 2H); 3.87 (s, 3H); 4.65 (s, 2H); 6.86 (d, 1H, J=7.5 Hz); 7.03 (d, 1H, J=2.5 Hz); 7.07 (dd, 1H, J=7.5 Hz, J=2.5 Hz).

### Example 12.54.

### 2-butyl-1-[(4-bromo-3-ethyl phenyl)methyl]-4-spirocyclopentyl-1H-imidazol-5(4H)-one

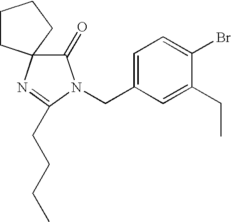

Prepared following the general procedure previously described (Method 12C) using 2-butyl-4-spirocyclopentyl-1H-imidazol-5(4H)-one (example 3.4) and 4-bromo-1-(bromomethyl)-3-ethylbenzene (example 10.6). The product was chromatographed over silica gel (eluent petroleum ether/ethyl acetate 80/20). The product was obtained as a colorless oil.

Yield: 70%

Rf (petroleum ether/ethyl acetate 70/30): 0.35

NMR ¹H (CDCl₃): 0.88 (t, 3H, J=7.5 Hz); 1.21 (t, 3H, J=7.5 Hz); 1.29-1.41 (m, 2H); 1.52-1.64 (m, 2H); 1.81-2.06 (m, 8H); 2.27-2.33 (m, 2H); 2.75 (q, 2H, J=7.5 Hz); 4.63 (s, 2H); 6.86 (dd, 1H, J=7.5 Hz, J=2.5 Hz); 7.03 (d, 1H, J=2.5 Hz); 7.50 J=7.5 Hz).

### Example 12.55.

### 2-butyl-1-[(4-(4,4,5,5-tetramethyl-[1,3,2]dioxaborolan-2-yl)phenyl)methyl]-4-spirocyclopentyl-1H-imidazol-5(4H)-one

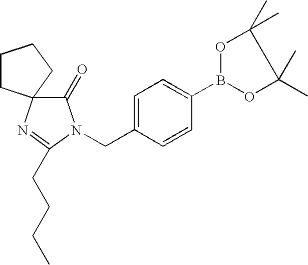

Prepared following the general procedure previously described (Method 12B) using 2-butyl-4-spirocyclopentyl-1H-imidazol-5(4H)-one (example 3.4) and 2-(4-(bromomethyl)phenyl)-4,4,5,5-tetramethyl-1,3,2-dioxaborolane The product was obtained as a brown oil and was used without any further purification.

NMR ¹H (CDCl₃): 0.83 (t, 3H, J=7.3 Hz); 1.32-1.41 (m, 14H); 1.49-1.55 (m, 2H); 1.77-1.96 (m, 8H); 2.39-2.45 (m, 2H); 4.68 (s, 2H); 7.13 (d, 2H, J=8.1 Hz); 7.76 (d, 2H, J=8.1 Hz).

### Example 12.56.

### 2-butyl-1-[(4-bromo-3-methoxyphenyl)methyl]-4-spirocyclopentyl-1H-imidazol-5(4H)-one

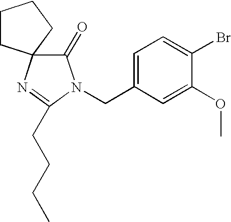

Prepared following the general procedure previously described (Method 12C) using 2-butyl-4-spirocyclopentyl-1H-imidazol-5(4H)-one (example 3.4) and 1-bromo-4-(bromomethyl)-2-methoxybenzene (example 10.8). The product was chromatographed over silica gel (elution gradient: petroleum ether/ethyl acetate 100/0 to 70/30). The product was obtained as a colorless oil.

Yield: 70%

Rf (petroleum ether/ethyl acetate 60/40): 0.41

NMR ¹H (CDCl₃): 0.88 (t, 3H, J=7.5 Hz); 1.31-1.39 (m, 2H); 1.56-1.62 (m, 2H); 1.84-2.05 (m, 8H); 2.27-2.33 (m, 2H); 3.73 (s, 3H); 4.75 (s, 2H); 6.46 (d, 1H, J=2.5 Hz); 6.72 (dd, 1H, J=7.5 Hz, J=2.5 Hz); 7.46 (d, 1H, J=7.5 Hz).

### Example 12.57.

### 2-butyl-1-[(4-bromo-2-methylphenyl)methyl]-4-spirocyclopentyl-1H-imidazol-5(4H)-one

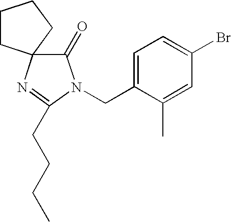

Prepared following the general procedure previously described (Method 12C) using 2-butyl-4-spirocyclopentyl-1H-imidazol-5(4H)-one (example 3.4) and 1-bromo-4-(bromomethyl)-3-methylbenzene (example 10.9). The product was chromatographed over silica gel (eluent: petroleum ether/ethyl acetate 80/20). The product was obtained as a white solid.

Yield: 69%

Rf (petroleum ether/ethyl acetate 70/30): 0.37

NMR ¹H (CDCl₃): 0.86 (t, 3H, J=7.5 Hz); 1.23-1.38 (m, 2H); 1.49-1.1.61 (m, 2H); 1.82-2.02 (m, 8H); 2.20-2.26 (m, 2H); 2.29 (s, 3H); 4.60 (s, 2H); 6.73 (d, 1H, J=7.5 Hz); 7.29 (d, 1H, J=7.5 Hz); 7.34 (s, 1 H).

### Example 12.58.

### 2-butyl-1-[(4-bromo-3-propylphenyl)methyl]-4-spirocyclopentyl-1H-imidazol-5(4H)-one

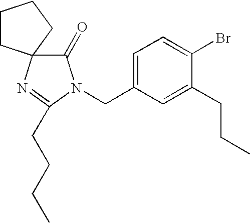

Prepared following the general procedure previously described (Method 12C) using 2-butyl-4-spirocyclopentyl-1H-imidazol-5(4H)-one (example 3.4) and 1-bromo-4-(bromomethyl)-2-propylbenzene (example 10.7). The product was chromatographed over silica gel (eluent: petroleum ether/ethyl acetate 80/20). The product was obtained as a colorless oil.

Yield: 70%

Rf (petroleum ether/ethyl acetate 70/30): 0.40

NMR ¹H (CDCl₃): 0.86 (t, 3H, J=7.5 Hz); 0.93 (t, 3H, J=7.5 Hz); 1.29-1.34 (m, 2H); 1.51-1.1.56 (m, 4H); 1.58-1.63 (m, 2H); 1.76-1.79 (m, 6H); 2.26-2.30 (m, 2H); 2.67 (t, 2H, J=7.5 Hz); 4.60 (s, 2H); 6.84 (dd, 1H, J=7.5 Hz, J=2.5 Hz); 6.99 (d, 1H, J=2.5 Hz); 7.47 (d, 1H, J=7.5 Hz).

### Example 12.59.

### 2-butyl-1-[(4-bromo-3-trifluoromethylphenyl)methyl]-4-spirocyclopentyl-1H-imidazol-5(4H)-one

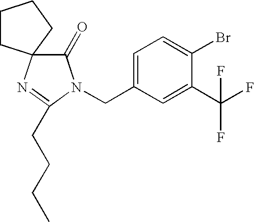

Prepared following the general procedure previously described (Method 12C) using 2-butyl-4-spirocyclopentyl-1H-imidazol-5(4H)-one (example 3.4) and 1-bromo-4-(bromomethyl)-2-trifluoromethylbenzene (example 10.10). The product was chromatographed over silica gel (eluent: petroleum ether/ethyl acetate 70/30). The product was obtained as a colorless oil.

Yield: 81%

Rf (petroleum ether/ethyl acetate 60/40): 0.46

NMR ¹H (CDCl₃): 0.85 (t, 3H, J=7.5 Hz); 1.24-1.39 (m, 2H); 1.50-1.62 (m, 2H); 1.78-1.95 (m, 8H); 2.24-2.30 (m, 2H); 4.65 (s, 2H); 7.17 (d, 1H, J=7.5 Hz); 7.45 (s, 1H); 7.67 (d, 1H, J=7.5 Hz).

### Example 12.60.

### 2-butyl-1-[(4-bromo-3-nitrophenyl)methyl]-4-spirocyclopentyl-1H-imidazol-5(4H)-one

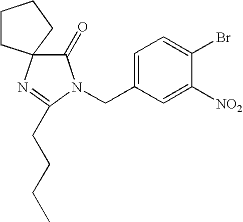

Prepared following the general procedure previously described (Method 12C) using 2-butyl-4-spirocyclopentyl-1H-imidazol-5(4H)-one (example 3.4) and 1-bromo-4-(bromomethyl)-2-nitrobenzene (example 10.11). The product was chromatographed over silica gel (eluent: petroleum ether/ethyl acetate 70/30). The product was obtained as a colorless oil.

Yield: 87%

Rf (petroleum ether/ethyl acetate 60/40): 0.33

NMR ¹H (CDCl₃): 0.90 (t, 3H, J=7.5 Hz); 1.32-1.43 (m, 2H); 1.57-1.67 (m, 2H); 1.81-2.05 (m, 8H); 2.29-2.35 (m, 2H); 4.70 (s, 2H); 7.26 (d, 1H, J=7.5 Hz); 7.67 (s, 1 H); 7.74 (d, 1H, J=7.5 Hz).

### Example 12.61.

### 2-butyl-1-[[2-[(4-methoxyphenyl]-6-methyl-thiazolo[3,2-b][1,2,4]triazol-5-yl]methyl]-4-spirocyclopentyl-1H-imidazol-5(4H)-one

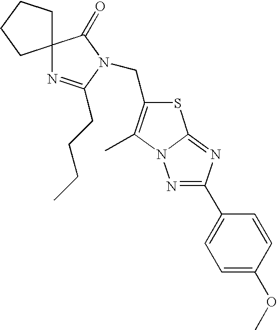

Prepared following the general procedure previously described (Method 12D) using 2-butyl-4-spirocyclopentyl-1H-imidazol-5(4H)-one (example 3.4) and 5-bromomethyl-2-[(4-methoxyphenyl]-6-methyl-thiazolo[3,2-b][1,2,4]triazole (example 6.11). The product was chromatographed over silica gel (elution gradient cyclohexane/ethyl acetate 80/20 to 50/50). The product was obtained as a colorless oil.

Yield: 64%

Rf (cyclohexane/ethyl acetate 50/50): 0.25

NMR ¹H (CDCl₃): 0.95 (t, 3H, J=7.3 Hz); 1.43 (m, 2H); 1.71 (m, 4H); 1.94 (m, 6H); 2.46 (t, 2H, J=7.3 Hz); 2.64 (s, 3H); 3.86 (s, 3H); 4.76 (s, 2H); 6.97 (d, 2H, J=8.8 Hz); 8.09 (d, 2H, J=8.8 Hz).

### Example 12.62.

### 2-butyl-1-[[2-[(3-methoxyphenyl]-6-methyl-thiazolo[3,2-b][1,2,4]triazol-5-yl]methyl]-4-spirocyclopentyl-1H-imidazol-5(4H)-one

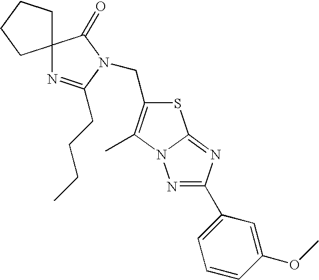

Prepared following the general procedure previously described (Method 12D) using 2-butyl-4-spirocyclopentyl-1H-imidazol-5(4H)-one (example 3.4) and 5-bromomethyl-2-[(3-methoxyphenyl]-6-methyl-thiazolo[3,2-b][1,2,4]triazole (example 6.12). The product was chromatographed over silica gel (elution gradient cyclohexane/ethyl acetate 80/20 to 50/50). The product was obtained as a colorless oil.

Yield: 64.2%

Rf (cyclohexane/ethyl acetate 50/50): 0.2

NMR ¹H (CDCl₃): 0.90 (t, 3H, J=7.3 Hz); 1.39 (m, 2H); 1.69 (m, 4H); 1.90 (m, 6H); 2.40 (t, 2H, J=7.9 Hz); 2.58 (s, 3H); 3.84 (s, 3H); 4.68 (s, 2H); 6.92 (dd, 1H, J=1.8 Hz, J=7.2 Hz); 7.31 (t, 1H); 7.65 (m, 1H); 7.71 (d, 1H, J=7.6 Hz).

### Example 13.

### General Procedure for the Preparation of Phenol

Method 13A: using the appropriate brominated derivative and the suitable hydroxyphenylboronic acid.

Preparation of the non commercially available boronic acids using corresponding bromobenzenes commercially available or prepared according to the methods previously described (Example 10)

Bromobenzene (1eq) was dissolved in tetrahydrofuran under inert atmosphere. The reaction mixture was cooled to −78° C., and then n-butyllithium (1.1eq) was added drop by drop. The mixture was stirred at −78° C. for 1 hour. Triisopropyl borate was added and the reaction mixture was then stirred at room temperature for 16 hours. Borate was hydrolyzed with water then tetrahydrofuran was partially evaporated at room temperature under reduced pressure. The residue was taken up in water and the mixture was acidified at 0° C. with a hydrochloric acid 1 M solution to reach pH 2, then extracted with ethyl acetate. The organic layer was washed with water, dried over magnesium sulfate and evaporated under reduced pressure at room temperature. The residue was taken up in petroleum ether and the mixture was cooled at −18° C. for a night. The resulting precipitate was filtered and used without any further purification.

Suzuki Reaction

Boronic acid (1eq, commercially available or prepared according to the method previously described), then brominated derivative (1 to 1.5eq), tetrakis palladium (0.03eq) and then aqueous potassium carbonate 1M solution (1eq to 3eq) was successively poured into 1,4-dioxane. The reaction mixture was stirred at reflux for 1 night. 1,4-dioxane was evaporated under reduced pressure. The residue was taken up in ethyl acetate and washed with brine. The organic layer was dried over sodium sulfate, filtered and evaporated under reduced pressure. The product was chromatographed over silica gel.

Method 13B: using the appropriate bromintade derivative and the phenylboronic acid with an alkylated hydroxyle function. The Suzuki reaction was followed by a deprotection of the alkylated hydroxyle.

Suzuki Reaction

Boronic acid (1eq, commercially available or prepared according to the method 13A previously described), then brominated derivative (1eq), tetrakis palladium (0.03eq) and then aqueous potassium carbonate 1M solution (1eq) was successively poured into 1,4-dioxane. The reaction mixture was stirred at reflux for 1 night. 1,4-dioxane was evaporated under reduced pressure. The residue was taken up in ethyl acetate and washed with brine. The organic layer was dried over sodium sulfate, filtered and evaporated under reduced pressure. The product was chromatographed over silica gel.

Demethylation Reaction

The methoxylated derivative previously prepared (1eq) was dissolved in chloroform. The mixture was cooled at 0° C., and then boron tribromide (2 to 9eq) was added drop by drop. The reaction mixture was slowly warmed to room temperature then stirred at room temperature for 8 hours. The mixture was poured into ice and extracted with dichloromethane. The organic layer was dried over sodium sulfate, filtered and evaporated under reduced pressure. The product was chromatographed over silca gel.

### Example 13.1.

### 2-butyl-1-[(3′-hydroxybiphenyl-4-yl)methyl]-4-spirocyclohexyl-1H-imidazol-5(4H)-one

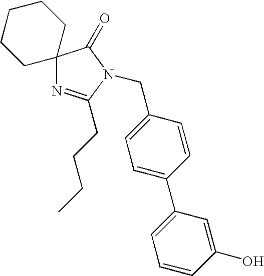

Prepared following the Suzuki reaction previously described (Method 13A) using 2-butyl-1-[(4-bromophenyl)methyl]-4-spirocyclohexyl-1H-imidazol-5(4H)-one (example 12.48) and 3-hydroxyphenylboronic acid. The product was chromatographed over silica gel (elution gradient cyclohexane/ethyl acetate 8/2 to 7/3). The product was obtained as a colorless oil.

Yield: 41.4%

Rf (cyclohexane/ethyl acetate 60/40): 0.4

IR: νCO 1712 cm⁻¹

NMR ¹H (CDCl₃): 0.77 (t, 3H, J=7.3 Hz); 1.25 (m, 2H); 1.67 (m, 12H); 2.38 (t, 2H J=7.9 Hz); 4.74 (s, 2H, J=1.7. 7.9 Hz); 6.86 (dd, 1H, J=8.2 Hz); 7.05 (m, 1H); 7.1 (m, 1H); 7.19 (d, 2H, J=8.2 Hz); 7.32 (t, 1H, J=7.2 Hz); 7.5 (m, 2H, J=8.2Hz).

### Example 13.2.

### 2-butyl-1-[(6′-fluoro-3′-hydroxybiphenyl-4-yl)methyl]-4-spirocyclohexyl-1H-imidazol-5(4H)-one

### 13.2.1

### 2-butyl-1-[(6′-fluoro-3′-methoxybiphenyl-4-yl)methyl]-4-spirocyclohexyl-1H-imidazol-5(4H)-one

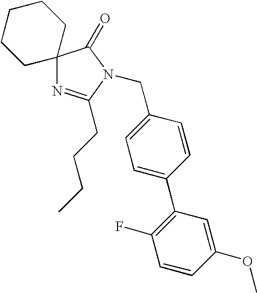

Prepared following the Suzuki reaction previously described (Method 13B) using 2-butyl-1-[(4-bromophenyl)methyl]-4-spirocyclohexyl-1H-imidazol-5(4H )-one (example 12.48) and 6-fluoro-3-methoxyphenylboronic acid. The product was chromatographed over silica gel (elution gradient cyclohexane/ethyl acetate 8/2 to 7/3). The product was obtained as a colorless oil.

Yield: 66.6%

Rf (cyclohexane/ethyl acetate 70/30): 0.3

IR: νCO 1721 cm⁻¹

NMR ¹H (CDCl₃): 0.88 (t, 3H, J=7.3 Hz); 1.35 (m, 2H); 1.67 (m, 12H); 2.37 (t, 2H, J=7.9 Hz); 3.83 (s, 3H); 4.73 (s, 2H); 6.84 (m, 1H); 6.92 (m, 1H); 7.08 (t, 1H, J=9.1 Hz); 7.23 (d, 2H, J=7.9 Hz); 7.52 (d, 2H, J=7.9 Hz).

### 13.2.2

### 2-butyl-1-[(6′-fluoro-3′-hydroxybiphenyl-4-yl)methyl]-4-spirocyclohexyl-1H-imidazol-5(4H )-one

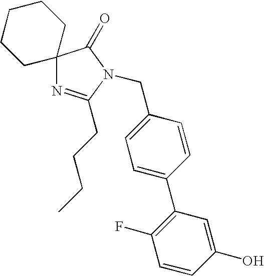

Prepared following the demethylation reaction previously described (Method 13B) using 2-butyl-1-[(6′-fluoro-3′-methoxybiphenyl-4-yl)methyl]-4-spirocyclohexyl-1H-imidazol-5(4H)-one (example 13.2.1). The product was chromatographed over silica gel (elution gradient dichloromethane/methanol 100/0 to 95/5). The product was obtained as a yellow powder.

Yield: 84.7%

Rf (dichloromethane/methanol 95/5): 0.3

IR: νCO 1728 cm⁻¹

NMR ¹H (CDCl₃): 0.79 (t, 3H, J=7.3 Hz); 1.27 (m, 2H); 1.67 (m, 12H); 2.39 (t, 2H, J=7.6 Hz); 4.74 (s, 2H); 6.8 (m, 1H); 6.86 (m, 1H); 7.02 (t, 1H, J=8.8 Hz); 7.19 (d, 2H, J=8.2 Hz); 7.47 (d, 2H, J=7 Hz); 7.73 (s, 1H).

### Example 13.3.

### 2-butyl-1-[(3′-hydroxybiphenyl-4-yl)methyl]-4,4-diethyl-1H-imidazol-5(4H)-one

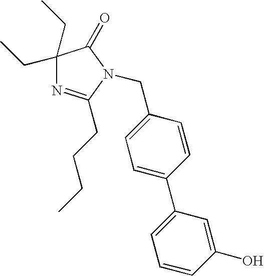

Prepared following the Suzuki reaction previously described (Method 13A) using 2-butyl-1-[(4-bromophenyl)methyl]-4,4-diethyl-1H-imidazol-5(4H )-one (example 12.49) and 3-hydroxyphenylboronic acid. The product was chromatographed over silica gel (elution gradient cyclohexane/ethyl acetate 8/2 to 7/3). The product was obtained as a white solid.

Yield: 45.9%

Rf (cyclohexane/ethyl acetate 60/40): 0.22

IR: νCO 1720 cm⁻¹

NMR ¹H (CDCl₃): 0.76 (m, 9H); 1.32 (m, 2H); 1.58 (m, 2H); 1.9 (q, 4H, J=7.3 Hz); 2.45 (t, 2H, J=7.9 Hz); 4.74 (s, 2H); 6.86 (dd, 1H, J=1.7, J=7.9 Hz); 7.05 (m, 1H); 7.09 (d, 1H, J=7.9 Hz); 7.31 (m, 3H); 7.51 (d, 2H, J=8.2 Hz); 7.65 (s, 1H).

### Example 13.4.

### 2-butyl-1-[(6′-fluoro-3′-hydroxybiphenyl-4-yl)methyl]-4,4-diethyl-1H-imidazol-5(4H)-one

### 13.4.1

### 2-butyl-1-[(6′-fluoro-3′-methoxybiphenyl-4-yl)methyl]-4,4-diethyl-1H-imidazol-5(4H)-one

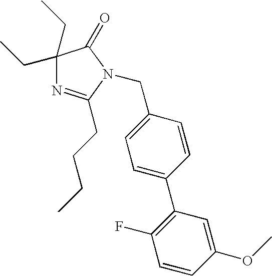

Prepared following the Suzuki reaction previously described (Method 13B) using 2-butyl-1-[(4-bromophenyl)methyl]-4.4-diethyl-1H-imidazol-5(4H)-one (example 12.49) and 6-fluoro-3-methoxyphenylboronic acid. The product was chromatographed over silica gel (elution gradient cyclohexane/ethyl acetate 8/2 to 7/3). The product was obtained as a yellow oil.

Yield: 50%

Rf (cyclohexane/ethyl acetate 60/40): 0.45

IR: νCO 1721 cm⁻¹

NMR ¹H (CDCl₃): 0.74 (t, 6H, J=7.6 Hz); 0.89 (t, 3H, J=7.3 Hz); 1.37 (m, 2H); 1.65 (m, 2H); 1.83 (q, 4H, J=7.3 Hz); 2.42 (t, 2H, J=7.2 Hz); 3.82 (s, 3H); 4.72 (s, 2H); 6.84 (m, 1H); 6.91 (m, 1H); 7.07 (m, 1H); 7.29 (d, 2H, J=8.5 Hz); 7.52 (d, 2H, J=7.3 Hz).

### 13.4.2

### 2-butyl-1-[(6′-fluoro-3′-hydroxybiphenyl-4-yl)methyl]-4.4-diethyl-1H-imidazol-5(4H)-one

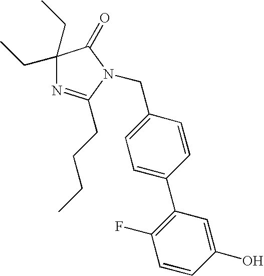

Prepared following the demethylation reaction previously described (Method 13B) using -butyl-1-[(6′-fluoro-3′-methoxybiphenyl-4-yl)methyl]-4-spirocyclohexyl-1H-imidazol-5(4H)-one (example 13.4.1). The product was chromatographed over silica gel (elution gradient cyclohexane/ethyl acetate 8/2 to 7/3). The product was obtained as a white solid.

Yield: 41.9%

Rf (cyclohexane/ethyl acetate 60/40): 0.2

IR: νCO 1738 cm⁻¹

NMR ¹H (CDCl₃): 0.74 (m, 9H); 1.27 (m, 2H); 1.55 (m, 2H); 1.89 (q, 4H, J=7.6 Hz); 2.45 (t, 2H, J=7.9 Hz); 4.73 (s, 2H); 6.83 (m, 2H); 7.02 (t, 1H, J=9.3 Hz); 7.27 (m, 2H); 7.47 (d, 2H, J=7.6 Hz); 8.1 (s, 1H).

### Example 13.5.

### 2-butyl-1-[(3′-hydroxy-2-methyl biphenyl-4-yl)methyl]-4-spirocyclohexyl-1H-imidazol-5(4H)-one

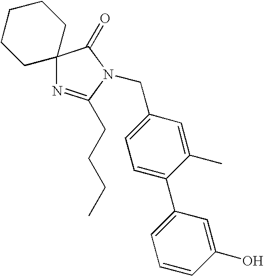

Prepared following the Suzuki reaction previously described (Method 13A) using 2-butyl-1-[(4-bromo-3-methylphenyl)methyl]-4-spirocyclohexyl-1H-imidazol-5(4H )-one (example 12.50) and 3-hydroxyphenylboronic acid. The product was chromatographed over silica gel (elution gradient cyclohexane/ethyl acetate 8/2 to 7/3). The product was obtained as a yellow solid.

Yield: 75%

Rf (cyclohexane/ethyl acetate 60/40): 0.4

IR: νCO 1727 cm⁻¹

NMR ¹H (CDCl₃): 0.8 (t, 3H, J=7.3 Hz); 1.29 (m, 2H); 1.69 (m, 12H); 2.23 (s, 3H); 2.42 (t, 2H, J=8.2 Hz); 4.69 (s, 2H); 6.83 (m, 3H); 7.02 (m, 2H); 7.16 (d, 1H, J=7.9 Hz); 7.28 (m, 1 H).

### Example 13.6.

### 2-butyl-1-[(3′-hydroxybiphenyl-3-yl)methyl]-4-spirocyclopentyl-1H-imidazol-5(4H)-one

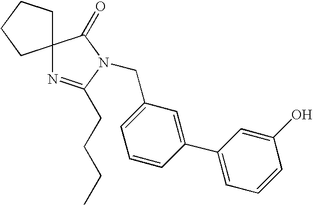

Prepared following the Suzuki reaction previously described (Method 13A) using 2-butyl-1-[(3-bromophenyl)methyl]-4-spirocyclopentyl-1H-imidazol-5(4H)-one (example 12.51) and 3-hydroxyphenylboronic acid. The product was chromatographed over silica gel (eluent cyclohexane/ethyl acetate 60/40). The product was obtained as a colorless oil.

Yield: 76%

Rf (petroleum ether/ethyl acetate 60/40): 0.36

IR: νCO 1722 cm⁻¹

NMR ¹H (CDCl₃): 0.77 (t, 3H, J=7.3 Hz); 1.25 (m, 2H); 1.53 (m, 2H); 1.85-2.07 (m, 8H); 2.35 (t, 2H, J=7.6 Hz); 4.75 (s, 2H); 6.84 (ddd, 1H, J=8 Hz, J=2.4 Hz, J=0.8 Hz); 7.03 (d, 1H, J=1.4 Hz); 7.04 (m, 1H); 7.09 (d, 1H, J=7.8 Hz); 7.23 (d, 1H, J=8.0 Hz); 7.33 (m, 1H); 7.36 (t, 1H, J=7.8 Hz); 7.45 (dt, 1H, J=7.6 Hz, J=1.7 Hz); 8.11 (s, 1H).

### Example 13.7.

### 2-butyl-1-[(2′-hydroxybiphenyl-3-yl)methyl]-4-spirocyclopentyl-1H-imidazol-5(4H)-one

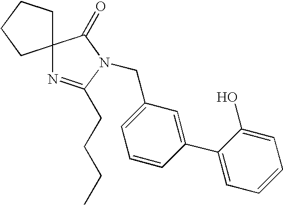

Prepared following the Suzuki reaction previously described (Method 13A) using 2-butyl-1-[(3-bromophenyl)methyl]-4-spirocyclopentyl-1H-imidazol-5(4H)-one (example 12.51) and 2-hydroxyphenylboronic acid. The product was chromatographed over silica gel (eluent cyclohexane/ethyl acetate 70/30). The product was obtained as a colorless oil.

Yield: 71%

Rf (petroleum ether/ethyl acetate 70/30): 0.43

IR: νCO 1724 cm⁻¹

NMR ¹H (CDCl₃): 0.78 (t, 3H, J=7.2 Hz); 1.25 (m, 2H, J=7.5 Hz); 1.55 (m, 2H, J=7.5 Hz); 1.80-2.04 (m, 8H); 2.34 (t, 2H, J=7.5 Hz); 4.73 (s, 2H); 6.94 (m, 2H); 7.10-7.25 (m, 3H); 7.35-7.45 (m, 3H); 7.63 (s, 1H).

### Example 13.8.

### 2-butyl-1-[(3′-hydroxy-6′-propylbiphenyl-4-yl)methyl]-4-spirocyclopentyl-1H-imidazol-5(4H)-one

### 13.8.1

### 2-butyl-1-[(3′-methoxy-6′-propylbiphenyl-4-yl)methyl]-4-spirocyclopentyl-1H-imidazol-5(4H)-one

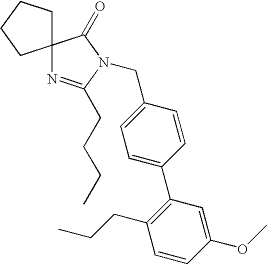

Prepared following the Suzuki reaction previously described (Method 13B) using 2-butyl-1-[(4-bromophenyl)methyl]-4-spirocyclopentyl-1H-imidazol-5(4H)-one (example 12.52) and 3-methoxy-6-propylphenylboronic acid (prepared following the method previously described (methode 13A) using 3-bromo-4-propylanisole

(example 10.2.2)). The product was chromatographed over silica gel (elution gradient petroleum ether/ethyl acetate 8/2 to 6/4). The product was obtained as a yellow oil.

Yield: 60%

Rf (petroleum ether/ethyl acetate 60/40): 0.5

IR: νCO 1719 cm⁻¹

NMR ¹H (CDCl₃): 0.78 (t, 3H, J=7.3 Hz); 0.86 (t, 3H, J=7.3 Hz); 1.28-1.47 (m, 4H); 1.52-1.64 (m, 2H); 1.81-2.04 (m, 8H); 2.31-2.38 (m, 2H); 2.41-2.48 (m, 2H); 3.79 (s, 3H); 4.73 (s, 2H); 6.71 (d, 1H, J=2.8 Hz); 6.85 (dd, 1H, J=8.4 Hz, J=2.8 Hz); 7.16-7.20 (m, 3H); 7.28 (d, 2H, J=8.4 Hz).

### 13.8.2

### 2-butyl-1-[(3′-hydroxy-6′-propylbiphenyl-4-yl )methyl]-4-spirocyclopentyl-1H-imidazol-5(4H)-one

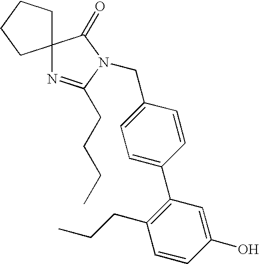

Prepared following the demethylation reaction previously described (Method 13B) using 2-butyl-1-[(3′-methoxy-6′-propylbiphenyl-4-yl)methyl]-4-spirocyclopentyl-1H-imidazol-5(4H)-one (example 13.8.1). The product was chromatographed over silica gel (elution gradient dichloromethane/methanol 100/0 to 95/5). The product was obtained as a beige powder.

Yield: 96%

Rf (petroleum ether/ethyl acetate 80/20): 0.55

IR: νCO 1726 cm⁻¹

NMR ¹H (CDCl₃): 0.71-0.80 (m, 6H); 1.16-1.54 (m, 6H); 1.82-2.03 (m, 8H); 2.33-2.44 (m, 4H); 4.72 (s, 2H); 6.65 (d,1H, J=2.6 Hz); 6.79 (dd,1H, J=8.3 Hz, J=2.6 Hz); 7.11-7.26 (m, 5H).

### Example 13.9.

### 2-butyl-1-[(4′-hydroxybiphenyl-3-yl)methyl]-4-spirocyclopentyl-1H-imidazol-5(4H)-one

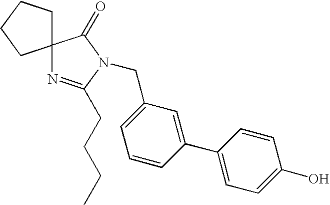

Prepared following the Suzuki reaction previously described (Method 13A) using 2-butyl-1-[(3-bromophenyl)methyl]-4-spirocyclopentyl-1H-imidazol-5(4H)-one (example 12.51) and 4-hydroxyphenylboronic acid. The product was chromatographed over silica gel (elution gradient petroleum ether/ethyl acetate 80/20 to 70/30). The product was obtained as a yellow oil.

Yield: 82%

Rf (petroleum ether/ethyl acetate 70/30): 0.5

IR: νCO 1725 cm⁻¹

NMR ¹H (CDCl₃): 0.83 (t, 3H, J=7.3 Hz); 1.23-1.34 (m, 2H); 1.51-1.63 (m, 2H); 1.82-2.06 (m, 8H); 2.32-2.38 (m, 2H); 4.76 (s, 2H); 6.61 (s, 1H); 6.9 (d, 2H, J=8.6 Hz); 7.09 (d,1H, J=7.4 Hz); 7.34-7.47 (m, 4H).

### Example 13.10.

### 2-butyl-1-[(2′-fluoro-3′-hydroxybiphenyl-4-yl)methyl]-4-spirocyclopentyl-1H-imidazol-5(4H)-one

### 13.10.1

### 2-butyl-1-[(2′-fluoro-3′-methoxybiphenyl-4-yl)methyl]-4-spirocyclopentyl-1H-imidazol-5(4H)-one

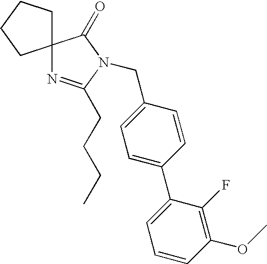

Prepared following the Suzuki reaction previously described (Method 13B) using 2-butyl-1-[(4-bromophenyl)methyl]-4-spirocyclopentyl-1H-imidazol-5(4H)-one (example 12.52) and 2-fluoro-3-methoxyphenylboronic acid. The product was chromatographed over silica gel (elution gradient petroleum ether/ethyl acetate 80/20 to 40/60). The product was obtained as a colorless oil.

Yield: 67%

Rf (petroleum ether/ethyl acetate 40/60): 0.5

IR: νCO 1721 cm⁻¹

NMR ¹H (CDCl₃): 0.86 (t, 3H, J=7.3 Hz); 1.26-1.41 (m, 2H); 1.52-1.64 (m, 2H); 1.81-2.05 (m, 8H); 2.31-2.37 (m, 2H); 3.92 (s, 3H); 4.72 (s, 2H); 6.92-7.00 (m, 2H); 7.10 (dd, 1H, J=8.0 Hz, J=1.2 Hz); 7.23 (d, 2H, J=8.2 Hz); 7.52 (dd, 2H, J=8.2 Hz, J=1.5 Hz).

### 13.10.2

### 2-butyl-1-[(2′-fluoro-3′-hydroxybiphenyl-4-yl)methyl]-4-spirocyclopentyl-1H-imidazol-5(4H)-one

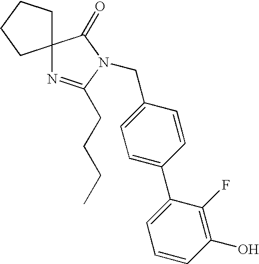

Prepared following the demethylation reaction previously described (Method 13B) using 2-butyl-1-[(2′-fluoro-3′-methoxybiphenyl-4-yl )methyl]-4-spirocyclopentyl-1H-imidazol-5(4H)-one (example 13.10.1). The product was chromatographed over silica gel (elution gradient dichloromethane/methanol 98/2). The product was obtained as a yellow powder.

Yield: 79%

Rf (petroleum ether/ethyl acetate 20/80): 0.6

NMR ¹H (CDCl₃): 0.78 (t, 3H, J=7.4 Hz); 1.19-1.28 (m, 2H); 1.47-1.56 (m, 2H); 1.86-2.06 (m, 8H); 2.33-2.40 (m, 2H); 4.73 (s, 2H); 6.82-6.88 (m, 1H); 6.92-7.05 (m, 2H); 7.21 (d, 2H, J=8.2 Hz); 7.48 (d, 2H, J=8.0 Hz).

### Example 13.11.

### 2-butyl-1-[(3′-hydroxy-4′-methoxybiphenyl-4-yl)methyl]-4-spirocyclopentyl-1H-imidazol-5(4H)-one

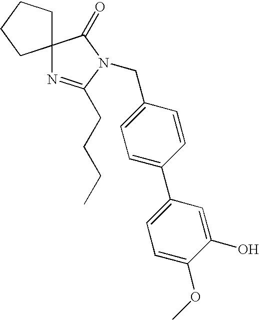

Prepared following the Suzuki reaction previously described (Method 13A) using 2-butyl-1-[(4-bromophenyl)methyl]-4-spirocyclopentyl-1H-imidazol-5(4H)-one (example 12.52) and 3-hydroxy-4-methoxyphenylboronic acid. The product was chromatographed over silica gel (elution gradient petroleum ether/ethyl acetate 80/20 to 60/40). The product was obtained as a pale yellow solid.

Yield: 69%

Rf (petroleum ether/ethyl acetate 40/60): 0.4

IR: νCO 1730 cm⁻¹

NMR ¹H (CDCl₃): 0.85 (t, 3H, J=7.3 Hz); 1.26-1.37 (m, 2H); 1.51-1.63 (m, 2H); 1.81-2.02 (m, 8H); 2.30-2.36 (m, 2H); 3.92 (s, 3H); 4.70 (s, 2H); 5.97 (sl, 1H); 6.91 (d, 1H, J=8.4 Hz); 7.06 (dd, 1H, J=8.3 Hz, J=2.2 Hz); 7.16-7.20 (m, 3H); 7.50 (d, 2H, J=8.2 Hz).

### Example 13.12.

### 2-butyl-1-[(6′-ethyl-3′-hydroxybiphenyl-4-yl)methyl]-4-spirocyclopentyl-1H-imidazol-5(4H)-one

### 13.12.1

### 2-butyl-1-[(6′-ethyl-3′-methoxybiphenyl-4-yl)methyl]-4-spirocyclopentyl-1H-imidazol-5(4H)-one

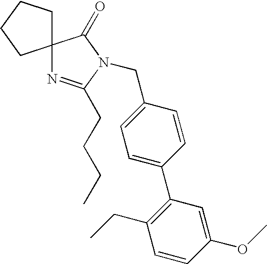

Prepared following the Suzuki reaction previously described (Method 13B) using 2-butyl-1-[(4-bromophenyl)methyl]-4-spirocyclopentyl-1H-imidazol-5(4H)-one (example 12.52) and 6-ethyl-3-methoxyphenylboronic acid (prepared following the method previously described (methode 13A) using 3-bromo-4-ethylanisole (example 10.1.2)). The product was chromatographed over silica gel (elution gradient petroleum ether /ethyl acetate 80/20 to 70/30). The product was obtained as a yellow oil.

Yield: 95%

Rf (petroleum ether/ethyl acetate 60/40): 0.35

IR: νCO 1718 cm⁻¹

NMR ¹H (CDCl₃): 0.85 (t, 3H, J=7.3 Hz); 1.04 (t, 3H, J=7.5 Hz); 1.29-1.38 (m, 2H); 1.54-1.62 (m, 2H); 1.82-2.05 (m, 8H); 2.34-2.37 (m, 2H); 2.47-2.52 (m, 2H); 3.78 (s, 3H); 4.73 (s, 2H); 6.72 (d, 1H, J=2.8 Hz); 6.86 (dd, 1H, J=8.7 Hz J=2.8 Hz); 7.18-7.21 (m, 2H); 7.29 (d, 3H, J=8.1 Hz).

### 13.12.2

### 2-butyl-1-[(6′-ethyl-3′-hydroxybiphenyl-4-yl)methyl]-4-spirocyclopentyl-1H-imidazol-5(4H)-one

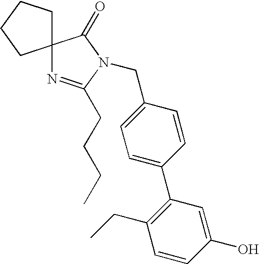

Prepared following the demethylation reaction previously described (Method 13B) using 2-butyl-1-[(6′-ethyl-3′-methoxybiphenyl-4-yl)methyl]-4-spirocyclopentyl-1H-imidazol-5(4H)-one (example 13.12.1). The product was chromatographed over silica gel (elution gradient dichloromethane/methanol 100/0 to 95/5). The product was obtained as a beige powder.

Yield: 91%

Rf (petroleum ether/ethyl acetate 20/80): 0.65

NMR ¹H (CDCl₃): 0.84 (t, 3H, J=7.3 Hz); 1.03 (t, 3H, J=7.5 Hz); 1.27-1.42 (m, 2H); 1.57-1.69 (m, 2H); 1.98-2.12 (m, 8H); 2.41-2.50 (m, 4H); 4.83 (s, 2H); 6.66 (d, 1H, J=2.7 Hz); 6.83 (dd, 1H, J=8.3 Hz, J=2.7 Hz); 7.14-7.18 (m, 3H); 7.29 (d, 2H, J=8.1 Hz).

### Example 13.13.

### 2-butyl-1-[(3′-hydroxy-4′-isobutylbiphenyl-4-yl)methyl]-4-spirocyclopentyl-1H-imidazol-5(4H)-one

### 13.13.1

### 2-butyl-1-[(4′-isobutyl-3′-methoxybiphenyl-4-yl)methyl]-4-spirocyclopentyl-1H-imidazol-5(4H)-one

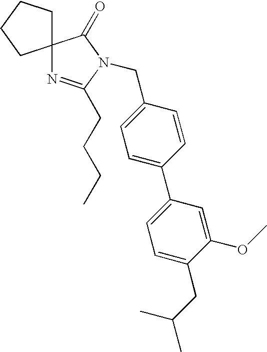

Prepared following the Suzuki reaction previously described (Method 13B) using 2-butyl-1-[(4-bromophenyl)methyl]-4-spirocyclopentyl-1H-imidazol-5(4H)-one (example 12.52) and 4-isobutyl-3-methoxyphenylboronic acid (prepared following the method previously described (methode 13A) using 3-bromo-6-isobutylanisole

(example 10.3.3)). The product was chromatographed over silica gel (elution gradient petroleum ether /ethyl acetate 80/20 to 70/30). The product was obtained as a yellow oil.

Yield: 79%

Rf (petroleum ether/ethyl acetate 60/40): 0.4

IR: νCO 1718 cm⁻¹

NMR ¹H (CDCl₃): 0.83-0.93 (m, 9H); 1.26-1.40 (m, 2H); 1.52-1.64 (m, 2H); 1.81-2.04 (m, 9H); 2.31-2.37 (m, 2H); 2.51 (d, 2H, J=7.1 Hz); 3.85 (s, 3H); 4.71 (s, 2H); 7.02-7.15 (m, 3H); 7.21 (d, 2H, J=8.1 Hz); 7.55 (d, 2H, J=8.1 Hz).

### 13.13.2

### 2-butyl-1-[(3′-hydroxy-4′-isobutyl-biphenyl-4-yl )methyl]-4-spirocyclopentyl-1H-imidazol-5(4H)-one

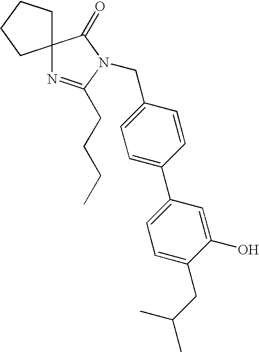

Prepared following the demethylation reaction previously described (Method 13B) using 2-butyl-1-[(4′-isobutyl-3′-methoxybiphenyl-4-yl)methyl]-4-spirocyclopentyl-1H-imidazol-5(4H)-one (example 13.13.1). The product was chromatographed over silica gel (eluent dichloromethane/methanol 95/5). The product was obtained as a yellow powder.

Yield: 92%

Rf (petroleum ether/ethyl acetate 20/80): 0.7

IR: νCO 1728 cm⁻¹

NMR ¹H (CDCl₃): 0.73 (t, 3H, J=7.3 Hz); 0.94 (d, 6H, J=6.6 Hz); 1.18-1.28 (m, 2H); 1.48-1.66 (m, 2H); 1.88-2.09 (m, 9H); 2.34-2.38 (m, 2H); 2.53 (d, 2H, J=7.1 Hz); 4.72 (s, 2H); 7.03 (m, 2H); 7.11-7.16 (m, 3H); 7.46 (d, 2H, J=8.3 Hz); 8.91 (s, 1H).

### Example 13.14.

### 2-butyl-1-[(3′-hydroxy-3-methoxybiphenyl-4-yl )methyl]-4-spirocyclopentyl-1H-imidazol-5(4H)-one

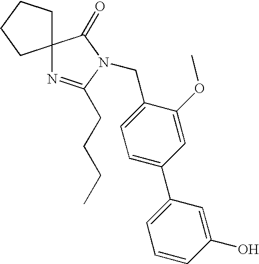

Prepared following the Suzuki reaction previously described (Method 13A) using 2-butyl-1-[(4-bromo-2-methoxyphenyl )methyl]-4-spirocyclopentyl-1H-imidazol-5(4H)-one (example 12.53) and 3-hydroxyphenylboronic acid. The product was chromatographed over silica gel (eluent petroleum ether/ethyl acetate 60/40). The product was obtained as a white solid.

Yield: 76%

Rf (petroleum ether/ethyl acetate 60/40): 0.22

IR: νCO 1727 cm⁻¹

NMR ¹H (CDCl₃): 0.80 (t, 3H, J=7.5 Hz); 1.22-1.37 (m, 2H); 1.51-1.63 (m, 2H); 1.88-2.11 (m, 8H); 2.40-2.46 (m, 2H); 3.88 (s, 3H); 4.78 (s, 2H); 6.88 (dd, 1H, J=7.5 Hz, J=1.3 Hz); 7.04-7.10 (m, 5H); 7.27-7.34 (m, 1H); 8.86 (s, 1H).

### Example 13.15.

### 2-butyl-1-[(3′-hydroxy-6′-isobutyl-biphenyl-4-yl)methyl]-4-spirocyclopentyl-1H-imidazol-5(4H)-one

### 13.15.1

### 2-butyl-1-[(6′-isobutyl-3′-methoxybiphenyl-4-yl)methyl]-4-spirocyclopentyl-1H-imidazol-5(4H)-one

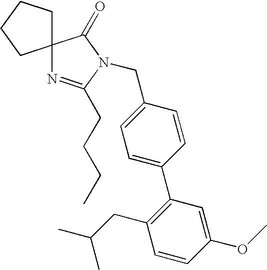

Prepared following the Suzuki reaction previously described (Method 13B) using 2-butyl-1-[(4-bromophenyl)methyl]-4-spirocyclopentyl-1H-imidazol-5(4H)-one (example 12.52) and 6-isobutyl-3-methoxyphenylboronic acid (prepared following the method previously described (methode 13A) using 3-bromo-4-isobutylanisole (example 10.3.2)). The product was chromatographed over silica gel (elution gradient petroleum ether/ethyl acetate 80/20 to 70/30). The product was obtained as a yellow oil.

Yield: 79%

Rf (petroleum ether/ethyl acetate 40/60): 0.6

IR: νCO 1724 cm⁻¹

NMR ¹H (CDCl₃): 0.70 (d, 6H, J=6.6 Hz); 0.86 (t, 3H, J=7.3 Hz); 1.28-1.38 (m, 2H); 1.54-1.62 (m, 3H); 1.83-2.07 (m, 8H); 2.32-2.39 (m, 4H); 3.78 (s, 3H); 4.74 (s, 2H); 6.71 (d, 1H, J=2.7 Hz); 6.83 (dd, 1H, J=8.4 Hz, J=2.7 Hz); 7.14 (d, 1H, J=8.4 Hz); 7.17 (d, 2H, J=8.0 Hz); 7.26 (d, 2H, J=8.0 Hz).

### 13.15.2

### 2-butyl-1-[(3′-hydroxy-6′-isobutyl-biphenyl-4-yl)methyl]-4-spirocyclopentyl-1H-imidazol-5(4H)-one

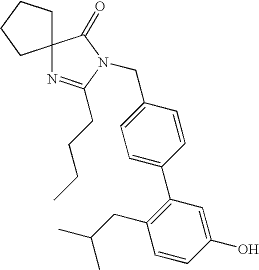

Prepared following the demethylation reaction previously described (Method 13B) using 2-butyl-1-[(6′-isobutyl-3′-methoxybiphenyl-4-yl)methyl]-4-spirocyclopentyl-1H-imidazol-5(4H)-one (example 13.15.1). The product was chromatographed over silica gel (elution gradient dichloromethane/methanol 100/0 to 98/2). The product was obtained as a yellow powder.

Yield: 100%

Rf (petroleum ether/ethyl acetate 20/80): 0.7

IR: νCO 1730 cm⁻¹

NMR ¹H (CDCl₃): 0.69-0.78 (m, 9H); 1.18-1.32 (m, 2H); 1.45-1.62 (m, 3H); 1.87-2.09 (m, 8H); 2.35-2.42 (m, 4H); 4.76 (s, 2H); 6.70 (d, 1H, J=2.6 Hz); 6.82 (dd, 1H, J=8.3 Hz, J=2.6 Hz); 7.08 (d, 1H, J=8.3 Hz); 7.16 (d, 2H, J=8.2 Hz); 7.24 (d, 2H, J=8.2 Hz); 9.01 (s, 1H).

### Example 13.16.

### 2-butyl-1-[(2-ethyl-3′-hydroxybiphenyl-4-yl)methyl]-4-spirocyclopentyl-1H-imidazol-5(4H)-one

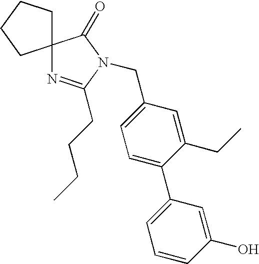

Prepared following the Suzuki reaction previously described (Method 13A) using 2-butyl-1-[(4-bromo-3-ethylphenyl)methyl]-4-spirocyclopentyl-1H-imidazol-5(4H)-one (example 12.54) and 3-hydroxyphenylboronic acid. The product was chromatographed over silica gel (eluent petroleum ether/ethyl acetate 80/20). The product was obtained as a beige solid.

Yield: 64%

Rf (petroleum ether/ethyl acetate 60/40): 0.48

IR: νCO 1732 cm⁻¹

NMR ¹H (CDCl₃): 0.73 (t, 3H, J=7.5 Hz); 1.04 (t, 3H, J=7.5 Hz); 1.20-1.35 (m, 2H); 1.49-1.58 (m, 2H); 1.86-2.08 (m, 8H); 2.38-2.45 (m, 2H); 2.58 (q, 2H); 4.73 (s, 2H); 6.77-6.80 (m, 2H); 2.87 (d, 1H, J=7.5 Hz); 7.01 (d, 1H, J=7.5 Hz); 7.07 (s, 1H); 7.15 (d, 1H, J=7.5 Hz); 7.27-7.30 (m, 1H); 8.55 (s,1H).

### Example 13.17.

### 2-butyl-1-[(6′-cyano-3′-hydroxybiphenyl-4-yl)methyl]-4-spirocyclopentyl-1H-imidazol-5(4H)-one

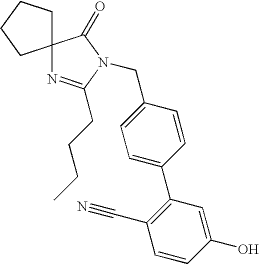

Prepared following the Suzuki reaction previously described (Method 13A) using 2-butyl-1-[(4-(4,4,5,5-tetramethyl-[1,3,2]dioxaborolan-2-yl)phenyl)methyl]-4-spirocyclopentyl-1H-imidazol-5(4H)-one (example 12.55) and 2-bromo-4-hydroxybenzonitrile (example 10.4). The product was chromatographed over silica gel (eluent petroleum ether/ethyl acetate 80/20 to 50/50). The product was obtained as a brown oil.

Yield: 30%

Rf (petroleum ether/ethyl acetate 40/60): 0.3

NMR ¹H (CDCl₃): 0.83 (t, 3H, J=7.3 Hz); 1.22-1.28 (m, 2H); 1.51-1.59 (m, 2H); 1.80-1.99 (m, 8H); 2.34-2.40 (m, 2H); 4.75 (s, 2H); 6.92 (dd, 1H, J=8.6 Hz, J=2.3 Hz); 6.95 (d, 1H, J=2.3 Hz); 7.24 (d, 2H, J=8.1 Hz); 7.51 (d, 2H, J=8.1 Hz); 7.56 (d,1H, J=8.6 Hz); 9.86 (s,1H).

### Example 13.18.

### 2-butyl-1-[(2-methoxy-3′-hydroxybiphenyl-4-yl)methyl]-4-spirocyclopentyl-1H-imidazol-5(4H)-one

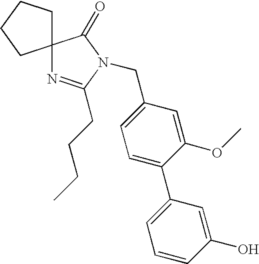

Prepared following the Suzuki reaction previously described (Method 13A) using 2-butyl-1-[(4-bromo-3-methoxyphenyl)methyl]-4-spirocyclopentyl-1H-imidazol-5(4H)-one (example 12.56) and 3-hydroxyphenylboronic acid. The product was chromatographed over silica gel (eluent petroleum ether/ethyl acetate 60/40). The product was obtained as a beige solid.

Yield: 78%

Rf (petroleum ether/ethyl acetate 60/40): 0.25

IR: νCO 1725 cm⁻¹

NMR ¹H (CDCl₃): 0.72 (t, 3H, J=7.5 Hz); 1.06-1.21 (m, 2H); 1.28-1.40 (m, 2H); 1.81-2.07 (m, 10H); 3.77 (s, 3H); 4.70 (s, 2H); 6.60 (s, 1H); 6.77-6.87 (m, 4H); 7.18-7.29 (m, 2H); 8.48 (s, 1H).

### Example 13.19.

### 2-butyl-1-[(3′-hydroxy-3-methyl-biphenyl-4-yl)methyl]-4-spirocyclopentyl-1H-imidazol-5(4H)-one

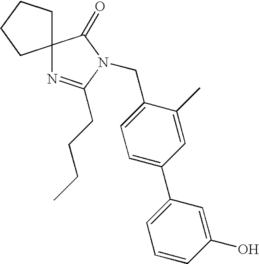

Prepared following the Suzuki reaction previously described (Method 13A) using 2-butyl-1-[(4-bromo-2-methylphenyl)methyl]-4-spirocyclopentyl-1H-imidazol-5(4H)-one (example 12.57) and 3-hydroxyphenylboronic acid. The product was chromatographed over silica gel (eluent petroleum ether/ethyl acetate 80/20). The product was obtained as a colorless oil.

Yield: 71%

Rf (petroleum ether/ethyl acetate 60/40): 0.5

IR: νCO 1718 cm⁻¹

NMR ¹H (CDCl₃): 0.78 (t, 3H, J=7.5 Hz); 1.20-1.35 (m, 2H); 1.50-1.62 (m, 2H); 1.95-2.14 (m, 8H); 2.32-2.38 (m, 5H); 4.76 (s, 2H); 6.85-6.93 (m, 2H); 7.09 (s, 1H); 7.12 (s, 1H); 7.27-7.35 (m, 2H); 7.41 (s, 1H); 8.64 (s, 1H).

### Example 13.20.

### 2-butyl-1-[[2-[(4-hydroxyphenyl]-6-methyl-thiazolo[3,2-b][1,2,4]triazol-5-yl]methyl]-4-spirocyclopentyl-1H-imidazol-5(4H)-one

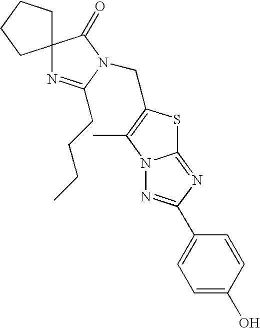

Prepared following the demethylation method previously described (Method 13B) using 2-butyl-1-[[2-[(4-methoxyphenyl]-6-methyl-thiazolo[3,2-b][1,2,4]triazol-5-yl]methyl]-4-spirocyclopentyl-1H-imidazol-5(4H)-one (example 12.61). The product was chromatographed over silica gel (elution gradient dichloromethane/methanol 90/10 to 70/30). The product was obtained as a white powder.

Yield: 70.2%

Rf (dichloromethane/methanol 90/10): 0.1

IR: νCO 1727 cm⁻¹

NMR ¹H (CDCl₃): 0.94 (t, 3H, J=7.6 Hz); 1.42 (m, 2H); 1.70 (m, 6H); 1.95 (m, 4H); 2.48 (t, 2H, J=8.2 Hz); 2.63 (s, 3H); 4.78 (s, 2H); 6.89 (d, 2H, J=8.8 Hz); 8.02 (d, 2H, J=8.8 Hz).

### Example 13.21.

### 2-butyl-1-[[2-[(3-hydroxyphenyl]-6-methyl-thiazolo[3,2-b][1,2,4]triazol-5-yl]methyl]-4-spirocyclopentyl-1H-imidazol-5(4H)-one

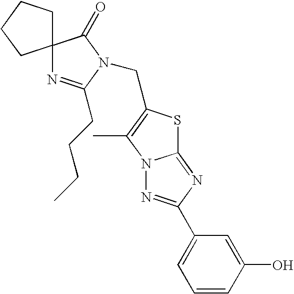

Prepared following the demethylation method previously described (Method 13B) using 2-butyl-1-[[2-[(3-methoxyphenyl]-6-methyl-thiazolo[3,2-b][1,2,4]triazol-5-yl]methyl]-4-spirocyclopentyl-1H-imidazol-5(4H)-one (example 12.62). The product was chromatographed over silica gel (eluent dichloromethane/methanol 95/5). The product was obtained as a white powder.

Yield: 63.9%

Rf (cyclohexane/ethyl acetate 50/50): 0.1

IR: νCO 1727 cm⁻¹

NMR ¹H (CDCl₃): 0.93 (t, 3H, J=7.3 Hz); 1.44 (m, 2H); 1.83 (m, 10H); 2.50 (t, 2H, J=7.6 Hz); 2.65 (s, 3H); 4.80 (s, 2H); 6.92 (m, 1H); 7.33 (t, 1H, J=7.9 Hz); 7.66 (m, 1H); 7.71 (d,1H, J=7.6 Hz).

### Example 13.22.

### 2-butyl-1-[(3′-hydroxy-2-propyl-biphenyl-4-yl)methyl]-4-spirocyclopentyl-1H-imidazol-5(4H)-one

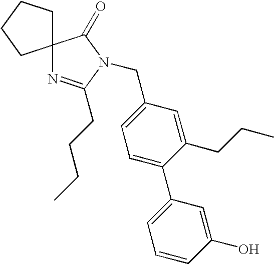

Prepared following the Suzuki reaction previously described (Method 13A) using 2-butyl-1-[(4-bromo-3-propyl phenyl)methyl]-4-spirocyclopentyl-1H-imidazol-5(4H)-one (example 12.58) and 3-hydroxyphenylboronic acid. The product was chromatographed over silica gel (eluent petroleum ether/ethyl acetate 80/20). The product was obtained as a colorless oil.

Yield: 68%

Rf (petroleum ether/ethyl acetate 60/40): 0.48

IR: νCO 1722 cm⁻¹

NMR ¹H (CDCl₃): 0.74-0.81 (m, 6H, J=7.5 Hz); 1.23-1.32 (m, 2H); 1.40-1.60 (m, 4H); 1.89-2.10 (m, 8H); 2.39-2.45 (m, 2H); 2.55 (t, 2H, J=7.5 Hz); 4.73 (s, 2H); 6.75-6.89 (m, 3H); 6.99 (d, 1H, J=7.5 Hz); 7.06 (s1, 1H); 7.14 (d, 1H, J=7.5 Hz); 7.24 (t, 1H, J=7.5 Hz); 9.12 (s, 1H).

### Example 13.23.

### 2-butyl-1-[(3′-hydroxy-6′-nitrobiphenyl-4-yl)methyl]-4-spirocyclopentyl-1H-imidazol-5(4H)-one

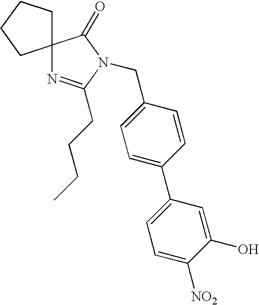

Prepared following the Suzuki reaction previously described (Method 13A) using 2-butyl-1-[(4-(4,4, 5, 5-tetramethyl-[1,3,2]dioxaborolan-2-yl)phenyl)methyl]-4-spirocyclopentyl-1H-imidazol-5(4H)-one (example 12.55) and 3-bromo-4-nitrophenol. The product was chromatographed over silica gel (eluent petroleum ether/ethyl acetate 80/20 to 60/40). The product was obtained as a yellow oil.

Yield: 33%

Rf (petroleum ether/ethyl acetate 60/40): 0.55

IR: νCO 1724 cm⁻¹

NMR ¹H (CDCl₃): 0.85 (t, 3H, J=7.3 Hz); 1.25-1.4 (m, 2H); 1.51-1.65 (m, 2H); 1.81-2.03 (m, 8H); 2.31-2.37 (m, 2H); 4.74 (s, 2H); 7.18 (dd, 1H, J=8.8 Hz, J=1.9 Hz); 7.28 (d, 2H, J=8.3 Hz); 7.32 (d, 1H, J=1.9 Hz); 7.59 (d, 2H, J=8.3 Hz); 8.14 (d, 1H, J=8.8 Hz); 10.66 (s, 1H).

### Example 13.24.

### 2-butyl-1-[(3′-hydroxy-2-trifluoromethylbiphenyl-4-yl)methyl]-4-spirocyclopentyl-1H-imidazol-5(4H)-one

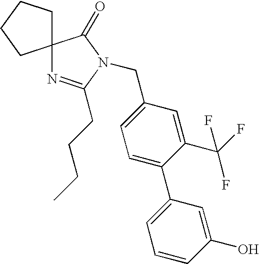

Prepared following the Suzuki reaction previously described (Method 13A) using 2-butyl-1-[(4-bromo-3-trifluoromethylphenyl)methyl]-4-spirocyclopentyl-1H-imidazol-5(4H)-one (example 12.59) and 3-hydroxyphenylboronic acid. The product was chromatographed over silica gel (eluent petroleum ether/ethyl acetate 70/30). The product was obtained as a yellow solid.

Yield: 75%

Rf (petroleum ether/ethyl acetate 60/40): 0.54

IR: νCO 1735 cm⁻¹

NMR ¹H (CDCl₃): 0.77 (t, 3H, J=7.5 Hz); 1.21-1.36 (m, 2H); 1.47-1.59 (m, 2H); 1.88-2.05 (m, 8H); 2.38-2.44 (m, 2H); 4.79 (s, 2H); 6.82 (s, 2H); 6.91 (d, 1H, J=7.5 Hz); 7.24-7.39 (m, 3H); 7.49 (s, 1H); 8.65 (s, 1H).

### Example 13.25.

### 2-butyl-1-[(3′-hydroxy-2-nitromethylbi phenyl-4-yl)methyl]-4-spirocyclopentyl-1H-imidazol-5(4H)-one

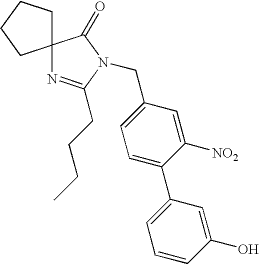

Prepared following the Suzuki reaction previously described (Method 13A) using 2-butyl-1-[(4-bromo-3-nitrophenyl)methyl]-4-spirocyclopentyl-1H-imidazol-5(4H)-one (example 12.60) and 3-hydroxyphenylboronic acid. The product was chromatographed over silica gel (eluent petroleum ether/ethyl acetate 70/30). The product was obtained as a beige solid.

Yield: 84%

Rf (petroleum ether/ethyl acetate 60/40): 0.23

IR: νCO 1741 cm⁻¹

NMR ¹H (CDCl₃): 0.83 (t, 3H, J=7.5 Hz); 1.30-1.42 (m, 2H); 1.55-1.68 (m, 2H); 1.88-2.07 (m, 8H); 2.44-2.50 (m, 2H); 4.81 (s, 2H); 6.79 (s, 1H); 1.84-1.94 (s, 2H); 7.32 (t, 1H, J=7.5 Hz); 7.47 (s, 2H); 7.57 (s, 1H); 9.16 (s, 1H).

### Example 13.26.

### 2-butyl-1-[(3′-hydroxy-4′-propylbiphenyl-4-yl)methyl]-4-spirocyclopentyl-1H-imidazol-5(4H)-one

### 13.26.1

### 2-butyl-1-[(3′-methoxy-4′-propylbiphenyl-4-yl)methyl]-4-spirocyclopentyl-1H-imidazol-5(4H)-one

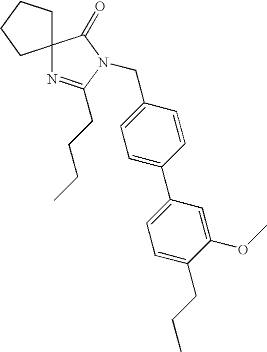

Prepared following the Suzuki reaction previously described (Method 13B) using 2-butyl-1-[(4-bromophenyl)methyl]-4-spirocyclopentyl-1H-imidazol-5(4H)-one (example 12.52) and 3-methoxy-4-propylphenylboronic acid (prepared following the method previously described (methode 13A) using 3-bromo-6-propylanisole (example 10.2.3)). The product was chromatographed over silica gel (elution gradient petroleum ether/ethyl acetate 80/20 to 60/40). The product was obtained as a colorless oil.

Yield: 82%

Rf (petroleum ether/ethyl acetate 60/40): 0.4

IR: νCO 1723 cm⁻¹

NMR ¹H (CDCl₃): 0.86 (t, 3H, J=7.3 Hz); 0.97 (t, 3H, J=7.3 Hz); 1.29-1.41 (m, 2H); 1.52-1.71 (m, 4H); 1.80-2.03 (m, 8H); 2.30-2.37 (m, 2H); 2.58-2.64 (m, 2H); 3.88 (s, 3H); 4.71 (s, 2H); 7.01 (d, 1H, J=1.4 Hz); 7.08 (dd, 1H, J=7.7 Hz, J=1.6 Hz); 7.17-7.23 (m, 3H); 7.54 (d, 2H, J=8.2 Hz).

### 13.26.2

### 2-butyl-1-[(3′-hydroxy-4′-propylbiphenyl-4-yl)methyl]-4-spirocyclopentyl-1H-imidazol-5(4H)-one

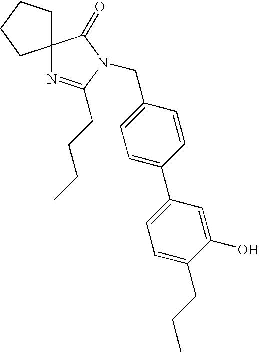

Prepared following the demethylation reaction previously described (Method 13B) using 2-butyl-1-[(3′-methoxy-4′-propylbiphenyl-4-yl)methyl]-4-spirocyclopentyl-1H-imidazol-5(4H)-one (example 13.26.1). The product was chromatographed over silica gel (elution gradient dichloromethane/methanol 100/0 to 95/5). The product was obtained as a yellow powder.

Yield: 93%

Rf (petroleum ether/ethyl acetate 20/80): 0.65

IR: νCO 1731 cm⁻¹

NMR ¹H (CDCl₃): 0.74 (t, 3H, J=7.3 Hz); 0.98 (t, 3H, J=7.3 Hz); 1.19-1.28 (m, 2H); 1.48-1.56 (m, 2H); 1.62-1.70 (m, 2H); 1.88-2.03 (m, 8H); 2.33-2.37 (m, 2H); 2.61-2.64 (m, 2H); 4.72 (s, 2H); 7.00-7.02 (m, 2H); 7.15-7.17 (m, 3H); 7.46 (d, 2H, J=8.2 Hz).

### Example 13.27.

### 2-butyl-1-[(3′-hydroxymethylbiphenyl-4-yl)methyl]-4-spirocyclopentyl-1H-imidazol-5(4H)-one

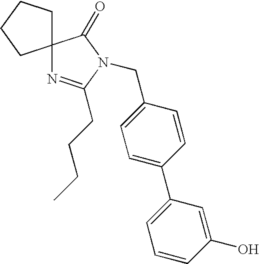

Prepared following the Suzuki reaction previously described (Method 13A) using 2-butyl-1-[(4-bromophenyl)methyl]-4-spirocyclopentyl-1H-imidazol-5(4H)-one (example 12.52) and 3-hydroxyphenylboronic acid. The product was chromatographed over silica gel (eluent petroleum ether/ethyl acetate 70/30). The product was obtained as a white solid.

Yield: 82%

Rf (petroleum ether/ethyl acetate 60/40): 0.25

IR: νCO 1730 cm⁻¹

NMR ¹H (CDCl₃): 0.79 (t, 3H, J=7.3 Hz); 1.21-1.36 (m, 2H); 1.51-1.62 (m, 2H); 1.90-12.13 (m, 8H); 2.37-2.34 (m, 2H); 4.77 (s, 2H); 6.87 (d, 1H, J=8.3 Hz); 7.08-7.11 (m, 2H); 7.21 (d, 2H, J=8 Hz); 7.27-7.33 (m, 1H); 7.52 (d, 2H, J=8 Hz); 8.95 (s, 1H).

### Example 14

### General Procedure for the Phenol O-alkylation

Method 14A: To a solution of phenol (1 eq) in acetonitrile was added potassium carbonate (3 to 6 eq), then brominated derivative (2 to 4 eq) drop by drop. The reaction mixture was stirred at reflux for 12 hours. Acetonitrile was evaporated under reduced pressure. The residue was taken up in water and extracted with dichloromethane. The organic layer was dried over sodium sulfate, filtered and evaporated under reduced pressure. The product was chromatographed over silica gel.

Method 14B: To a solution of phenol (1 eq) in acetonitrile was added potassium carbonate (3 eq), then brominated derivative (2 eq) drop by drop. The reaction mixture was stirred at reflux for 12 hours. Potassium carbonate was filtered and acetonitrile was evaporated under reduced pressure. The residue was chromatographed over silica gel.

Method 14C: To a solution of phenol (1 eq) in acetonitrile was added potassium carbonate (3 eq), then brominated derivative (2 eq) drop by drop. The reaction mixture was stirred at reflux for 12 hours. The reaction mixture was filtered then were added again potassium carbonate (3 eq), then brominated derivative (2 eq). The reaction mixture was stirred again at reflux for 12 hours. Potassium carbonate was filtered and acetonitrile was evaporated under reduced pressure. The residue was chromatographed over silica gel.

### Example 14.1.

### 2-butyl-1-[(3′-((1-ethoxycarbonylmethyl)oxy)biphenyl-4-yl)methyl]-4-spirocyclohexyl-1H-imidazol-5(4H)-one

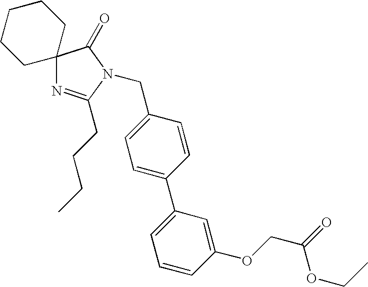

Prepared following the general procedure previously described (Method 14A) using 2-butyl-1-[(3′-hydroxybiphenyl-4-yl)methyl]-4-spirocyclohexyl-1H-imidazol-5(4H)-one (example 13.1) and ethyl bromoacetate. The product was chromatographed over silica gel (eluent cyclohexane/ethyl acetate 8/2). The product was obtained as a colorless oil.

Yield: 61.5%

Rf (cyclohexane/ethyl acetate 70/30): 0.25

IR: νCO 1721 and 1759 cm⁻¹

NMR ¹H (CDCl₃): 0.87 (t, 3H, J=7.3 Hz); 1.34 (m, 5H); 1.63 (m, 12H); 2.35 (t, 2H, J=8.2 Hz); 4.29 (q, 2H, J=7.3 Hz); 4.68 (s, 2H); 4.71 (s, 2H); 6.89 (dd, 1H, J=1.8 Hz, J=8.2 Hz); 7.13 (dd, 1H, J=1.8 Hz); 7.21 (m, 3H); 7.36 (t, 1H, J=7.9 Hz); 7.53 (d, 2H, J=8.2 Hz).

### Example 14.2.

### 2-butyl-1-[(3′-((1-ethoxycarbonyl-1-methylmethyl)oxy)-biphenyl-4-yl)methyl]-4-spirocyclohexyl-1H-imidazol-5(4H)-one

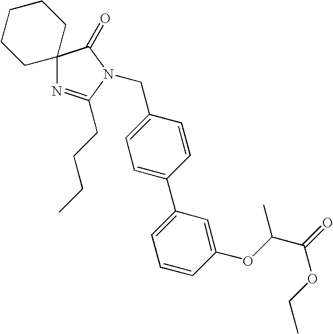

Prepared following the general procedure previously described (Method 14B) using 2-butyl-1-[(3′-hydroxybiphenyl-4-yl)methyl]-4-spirocyclohexyl-1H-imidazol-5(4H)-one (example 13.1) and ethyl 2-bromopropanoate. The product was chromatographed over silica gel (elution gradient cyclohexane/ethyl acetate 9/1 to 7/3). The product was obtained as a yellow oil.

Yield: 83.8%

Rf (cyclohexane/ethyl acetate 60/40): 0.55

IR: νCO 1723 and 1752 cm⁻¹

NMR ¹H (CDCl₃): 0.87 (t, 3H, J=7.3 Hz); 1.26 (t, 3H, J=7.3 Hz); 1.35 (m, 2H); 1.66 (m, 15H); 2.35 (t, 2H, J=8.2 Hz); 4.23 (q, 2H, J=7 Hz); 4.71 (s, 2H); 4.81 (q, 1H, J=7 Hz); 6.85 (dd, 1H, J=8.2 Hz, J=2 Hz); 7.11 (m, 1H); 7.19 (m, 3H); 7.33 (t, 1H, J=7.9 Hz); 7.53 (d, 2H, J=8.2 Hz).

### Example 14.3.

### 2-butyl-1-[(3′-((1-ethoxycarbonyl-1-ethylmethyl)oxy)-biphenyl-4-yl)methyl]-4-spirocyclohexyl-1H-imidazol-5(4H)-one

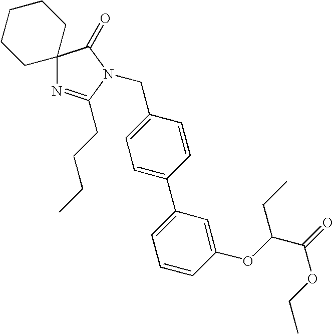

Prepared following the general procedure previously described (Method 14B) using 2-butyl-1-[(3′-hydroxybiphenyl-4-yl)methyl]-4-spirocyclohexyl-1H-imidazol-5(4H)-one (example 13.1) and ethyl 2-bromobutanoate. The product was chromatographed over silica gel (elution gradient cyclohexane/ethyl acetate 9/1 to 7/3). The product was obtained as a yellow oil.

Yield: 84.3%

Rf (cyclohexane/ethyl acetate 60/40): 0.55

IR: νCO 1724 and 1752 cm⁻¹

NMR ¹H (CDCl₃): 0.88 (t, 3H, J=7.3 Hz); 1.12 (t, 3H, J=76 Hz); 1.26 (t, 3H, J=7 Hz); 1.37 (m, 2H); 1.69 (m, 14H); 2.03 (m, 2H); 2.37 (t, 2H, J=8.2 Hz); 4.24 (q, 2H, J=7 Hz); 4.62 (q, 1H, J=7 Hz); 4.72 (s, 2H); 6.86 (dd, 1H, J=8.2 Hz, J=2 Hz); 7.12 (m, 1H); 7.21 (m, 3H); 7.34 (t, 1H, J=7.9 Hz); 7.52 (d, 2H, J=8.2 Hz).

### Example 14.4.

### 2-butyl-1-[(3′-((1-ethoxycarbonyl-1-(1,1-dimethylmethyl)-methyl)oxy)biphenyl-4-yl)methyl]-4-spirocyclohexyl-1H-imidazol-5(4H)-one

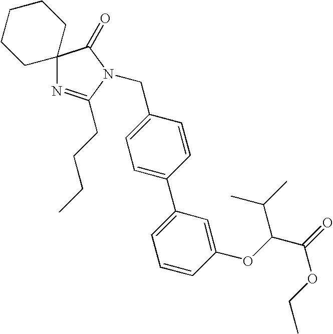

Prepared following the general procedure previously described (Method 14C) using 2-butyl-1-[(3′-hydroxybiphenyl-4-yl)methyl]-4-spirocyclohexyl-1H-imidazol-5(4H)-one (example 13.1) and ethyl 2-bromoisovalerate. The product was chromatographed over silica gel (elution gradient cyclohexane/ethyl acetate 9/1 to 7/3). The product was obtained as a yellow oil.

Yield: 87.2%

Rf (cyclohexane/ethyl acetate 60/40): 0.6

IR: νCO 1725 and 1751 cm⁻¹

NMR ¹H (CDCl₃): 0.88 (t, 3H, J=7.3 Hz); 1.11 (m, 6H); 1.26 (t, 3H, J=7.3 Hz); 1.35 (m, 2H); 1.71 (m, 12H); 2.33 (m, 3H); 4.24 (q, 2H, J=7.3 Hz); 4.42 (d, 1H, J=5.9 Hz); 4.73 (s, 2H); 6.86 (dd, 1H, J=8.2 Hz, J=1.7 Hz); 7.13 (m, 1H); 7.21 (m, 3H); 7.34 (t, 1. J=8.9 Hz); 7.54 (d, 2H, J=8.2 Hz).

### Example 14.5.

### 2-butyl-1-[(3′-((1-ethoxycarbonylmethyl)oxy)-6′-fluoro-biphenyl-4-yl)methyl]-4-spirocyclohexyl-1H-imidazol-5(4H)-one

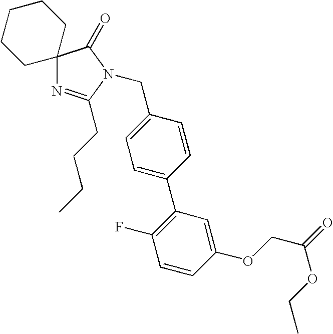

Prepared following the general procedure previously described (Method 14B) using 2-butyl-1-[(6′-fluoro-3′-hydroxybiphenyl-4-yl )methyl]-4-spirocyclohexyl-1H-imidazol-5(4H)-one (example 13.2) and ethyl 2-bromoacetate. The product was chromatographed over silica gel (elution gradient cyclohexane/ethyl acetate 1/0 to 7/3). The product was obtained as a colorless oil.

Yield: 83.8%

Rf (cyclohexane/ethyl acetate 60/40):0.5

IR: νCO 1721 and 1758 cm⁻¹

NMR ¹H (CDCl₃): 0.87 (t, 3H, J=7.3 Hz); 1.35 (m, 5H); 1.61 (m, 12H); 2.36 (t, 2H, J=7.6 Hz); 4.27 (q, 2H, J=7 Hz); 4.62 (s, 2H); 4.71 (s, 2H); 6.83 (m, 1H); 6.95 (m, 1H); 7.07 (m,1H); 7.22 (d, 2H, J=8.2 Hz); 7.49 (d, 2H, J=7 Hz).

### Example 14.6.

### 2-butyl-1-[(3′-((1-ethoxycarbonyl-1-methylmethyl)oxy)-6′-fluoro-biphenyl-4-yl)methyl]-4-spirocyclohexyl-1H-imidazol-5(4H)-one

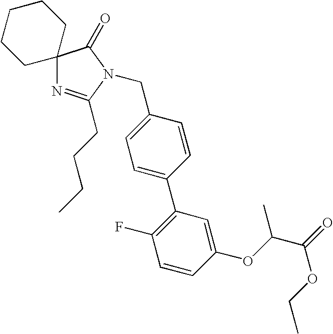

Prepared following the general procedure previously described (Method 14B) using 2-butyl-1-[(6′-fluoro-3′-hydroxybiphenyl-4-yl)methyl]-4-spirocyclohexyl-1H-imidazol-5(4H)-one (example 13.2) and ethyl 2-bromopropanoate. The product was chromatographed over silica gel (elution gradient cyclohexane/ethyl acetate 1/0 to 7/3). The product was obtained as a colorless oil.

Yield: 61.4%

Rf (cyclohexane/ethyl acetate 70/30): 0.45

IR: νCO 1724 cm⁻¹

NMR ¹H (CDCl₃): 0.87 (t, 3H, J=7.3 Hz); 1.26 (t, 3H, J=7 Hz); 1.33 (m, 2H); 1.64 (m, 15 Hz); 2.36 (t, 2H, J=7.9 Hz); 4.22 (q, 2H, J=7 Hz); 4.72 (m, 3H); 6.81 (m, 1H); 6.94 (m,1H); 7.05 (t, 1H, J=9.7 Hz); 7.22 (d, 2H, J=8.2 Hz); 7.49 (d, 2H, J=6.9 Hz).

### Example 14.7.

### 2-butyl-1-[(3′-((1-ethoxycarbonyl-1-ethylmethyl)oxy)-6′-fluoro-biphenyl-4-yl)methyl]-4-spirocyclohexyl-1H-imidazol-5(4H)-one

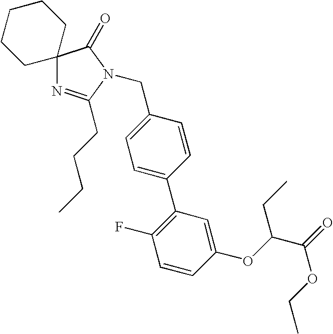

Prepared following the general procedure previously described (Method 14B) using 2-butyl-1-[(6′-fluoro-3′-hydroxybiphenyl-4-yl )methyl]-4-spirocyclohexyl-1H-imidazol-5(4H)-one (example 13.2) and ethyl 2-bromobutanoate. The product was chromatographed over silica gel (elution gradient cyclohexane/ethyl acetate 1/0 to 7/3). The product was obtained as a colorless oil.

Yield: 33.5%

Rf (cyclohexane/ethyl acetate 60/40): 0.4

IR: νCO 1724 cm⁻¹

NMR ¹H (CDCl₃): 0.87 (t, 3H, J=7.3 Hz); 1.09 (t, 3H, J=7.6 Hz); 1.25 (t, 3H, J=7.3 Hz); 1.36 (m, 2H); 1.63 (m, 12H); 1.99 (m, 2H); 2.36 (t, 2H, J=7.6 Hz); 4.23 (q, 2H, J=7 Hz); 4.53 (t, 1H, J=6.2 Hz); 4.72 (s, 2H); 6.81 (m, 1H); 6.94 (m, 1H); 7.05 (t,1H, J=9.1 Hz); 7.22 (d, 2H, J=8.2 Hz9); 7.49 (d, 2H, J=7 Hz).

### Example 14.8.

### 2-butyl-1-[(3′-((1-ethoxycarbonyl-1-(1,1-dimethylmethyl)-methyl)oxy)-6′-fluoro-biphenyl-4-yl)methyl]-4-spirocyclohexyl-1H-imidazol-5(4H)-one

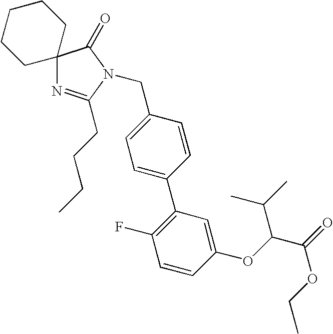

Prepared following the general procedure previously described (Method 14B) using 2-butyl-1-[(6′-fluoro-3′-hydroxybiphenyl-4-yl )methyl]-4-spirocyclohexyl-1H-imidazol-5(4H)-one (example 13.2) and ethyl 2-bromoisovalerate. The product was chromatographed over silica gel (elution gradient cyclohexane/ethyl acetate 1/0 to 7/3). The product was obtained as a colorless oil.

Yield: 75%

Rf (cyclohexane/ethyl acetate 60/40): 0.4

IR: νCO 1725 cm⁻¹

NMR ¹H (CDCl₃): 0.88 (t, 3H, J=7.3 Hz); 1.11 (m, 6H, J=6.4 Hz); 1.25 (t, 3H, J=7.3 Hz); 1.36 (m, 2H, J=7.6 Hz); 1.56 (m, 12H); 2.31 (m, 3H); 4.23 (q, 2H, J=7 Hz); 4.34 (d, 1H, J=5.6 Hz); 4.72 (s, 2H); 6.81 (m, 1H); 6.94 (m, 1H); 7.05 (t, 1H, J=9.7 Hz); 7.21 (d, 2H, J=7.9 Hz); 7.49 (d, 2H, J=7 Hz).

### Example 14.9.

### 2-butyl-1-[(3′-((1-ethoxycarbonyl-1,1-dimethylmethyl)oxy)-6′-fluoro-biphenyl-4-yl)methyl]-4-spirocyclohexyl-1H-imidazol-5(4H)-one

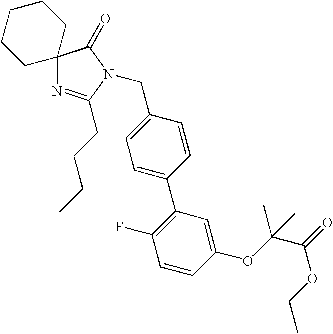

Prepared following the general procedure previously described (Method 14C) using 2-butyl-1-[(6′-fluoro-3′-hydroxybiphenyl-4-yl )methyl]-4-spirocyclohexyl-1H-imidazol-5(4H)-one (example 13.2) and ethyl 2-bromoisobutyrate. The product was chromatographed over silica gel (elution gradient cyclohexane/ethyl acetate 8/2 to 7/3). The product was obtained as a colorless oil.

Yield: 89.2%

Rf (cyclohexane/ethyl acetate 60/40): 0.55

IR: νCO 1727 cm⁻¹

NMR ¹H (CDCl₃): 0.86 (t, 3H, J=7.3 Hz); 1.25 (t, 3H, J=7 Hz); 1.34 (m, 2H); 1.63 (m, 18H); 2.35 (t, 2H, J=7.9 Hz); 4.23 (q, 2H, J=7 Hz); 4.71 (s, 2H); 6.82 (m, 1H); 6.98 (m, 2H); 7.21 (d, 2H, J=8.2 Hz); 7.47 (d, 2H, J=7.3 Hz).

### Example 14.10.

### 2-butyl-4-spirocyclohexyl-1-[(3′-((1-(cyano)methyl)oxy)-biphenyl-4-yl)methyl]-1H-imidazol-5(4H)-one

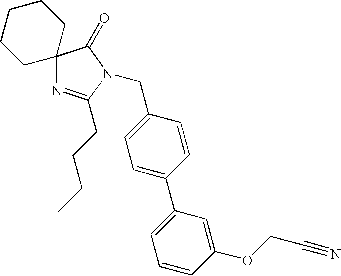

Prepared following the general procedure previously described (Method 14B) using 2-butyl-1-[(3′-hydroxybiphenyl-4-yl)methyl]-4-spirocyclohexyl-1H-imidazol-5(4H)-one (example 13.1) and 2-bromoacetonitrile. The product was chromatographed over silica gel (eluent cyclohexane/ethyl acetate 7/3). The product was obtained as a colorless oil.

Yield: 90.9%

Rf (cyclohexane/ethyl acetate 60/40): 0.45

IR: νCO 1716 cm⁻¹

NMR ¹H (CDCl₃): 0.89 (t, 3H, J=7.3 Hz); 1.33 (m, 2H); 1.65 (m, 12H, J=8.2 Hz); 2.38 (m, 2H); 4.74 (s, 2H); 4.85 (s, 2H); 6.99 (dd, 1J, J=7.9 Hz, J=2 Hz); 7.18 (m, 1H); 7.26 (m, 4H); 7.44 (t,1H, J=7.9 Hz); 7.56 (d, 2H, J=8.2 Hz).

### Example 14.11.

### 2-butyl-1-[(6′-fluoro-3′-((1-(cyano)methyl)oxy)biphenyl-4-yl)methyl]-4-spirocyclohexyl-1H-imidazol-5(4H)-one

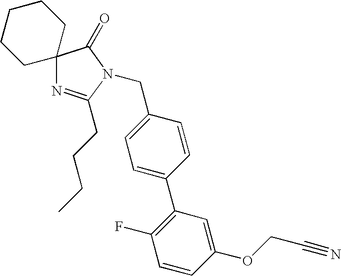

Prepared following the general procedure previously described (Method 14B) using 2-butyl-1-[(6′-fluoro-3′-hydroxybiphenyl-4-yl )methyl]-4-spirocyclohexyl-1H-imidazol-5(4H)-one (example 13.2) and 2-bromoacetonitrile. The product was chromatographed over silica gel (eluent cyclohexane/ethyl acetate 7/3). The product was obtained as a colorless oil.

Yield: 90.1%

Rf (cyclohexane/ethyl acetate 60/40): 0.45

IR: νCO 1760 cm⁻¹

NMR ¹H (CDCl₃): 0.87 (t, 3H, J=7.3 Hz); 1.35 (m, 2H); 1.63 (m, 12H); 2.36 (t, 2H, J=8.2 Hz); 4.72 (s, 2H); 4.78 (s, 2H); 6.94 (m, 1H); 7.02 (m, 1H); 7.14 (t, 1H, J=9.4 Hz); 7.23 (d, 2H, J=7.9 Hz); 7.51 (d, 2H, J=7.3 Hz).

### Example 14.12.

### 2-butyl-1-[(3′-((1-ethoxycarbonyl-1-(1,1-dimethylmethyl)-methyl)oxy)-biphenyl-4-yl)methyl]-4,4-diethyl-1H-imidazol-5(4H)one

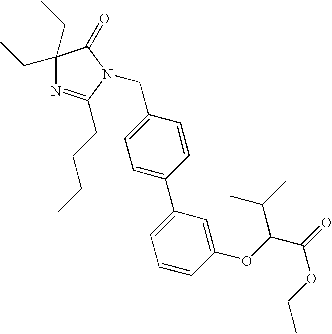

Prepared following the general procedure previously described (Method 14C) using 2-butyl-1-[(3′-hydroxybiphenyl-4-yl)methyl]-4,4-diethyl-1H-imidazol-5(4H)one (example 13.3) and ethyl 2-bromoisovalerate. The product was chromatographed over silica gel (elution gradient cyclohexane/ethyl acetate 9/1 to 7/3). The product was obtained as a colorless oil.

Yield: 71.7%

Rf (cyclohexane/ethyl acetate 60/40): 0.45

IR: νCO 1726 and 1751 cm⁻¹

NMR ¹H (CDCl₃): 0.73 (t, 6H, J=7.6 Hz); 0.88 (t, 3H, J=7.3 Hz); 1.09 (t, 6H, J=8.5 Hz); 1.24 (t, 3H, J=7 Hz); 1.36 (m, 2H); 1.64 (m, 2H); 1.83 (q, 4H, J=7.3 Hz); 2.29 (m, 1H); 2.4 (t, 2H, J=7.9 Hz); 4.22 (q, 2H, J=7 Hz); 4.41 (d, 1H, J=5.6 Hz); 4.7 (s, 2H); 6.84 (dd, 1H, J=7.9 Hz, J=1.8 Hz); 7.17 (m, 2H); 7.29 (m, 3H); 7.53 (d, 2H, J=8.2 Hz).

### Example 14.13.

### 2-butyl-1-[(3′-((1-ethoxycarbonyl-1-ethylmethyl)oxy)-biphenyl-4-yl)methyl]-4,4-diethyl-1H-imidazol-5(4H)one

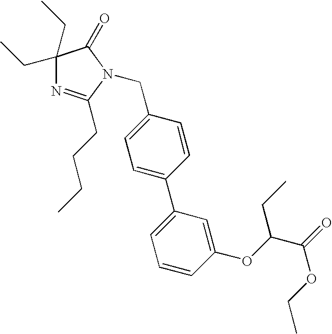

Prepared following the general procedure previously described (Method 14B) using 2-butyl-1-[(3′-hydroxybiphenyl-4-yl)methyl]-4,4-diethyl-1H-imidazol-5(4H)one (example 13.3) and ethyl 2-bromobutanoate. The product was chromatographed over silica gel (elution gradient cyclohexane/ethyl acetate 9/1 to 7/3). The product was obtained as a colorless oil.

Yield: 71.9%

Rf (cyclohexane/ethyl acetate 60/40): 0.4

IR: νCO 1726 and 1753 cm⁻¹

NMR ¹H (CDCl₃): 0.73 (t, 6H, J=7.6 Hz); 0.88 (t, 3H, J=7.3 Hz); 1.11 (t, 3H, J=7.3 Hz); 1.25 (t, 3H, J=7 Hz); 1.39 (m, 2H); 1.64 (m, 2H); 1.81 (q, 4H, J=7.3 Hz); 1.99 (m, 2H); 2.4 (t, 2H, J=7.9 Hz); 4.23 (q, 2H, J=7 Hz); 4.62 (t, 1H, J=6.4 Hz); 4.7 (s, 2H); 6.85 (dd, 1H, J=7.9 Hz, J=1.8 Hz); 7.12 (m, 1H); 7.18 (m, 1H); 7.31 (m, 3H); 7.53 (d, 2H, J=8.2 Hz).

### Example 14.14.

### 2-butyl-1-[(3′-((1-ethoxycarbonyl-1,1-dimethylmethyl)oxy)-biphenyl-4-yl)methyl]-4,4-diethyl-1H-imidazol-5(4H)one

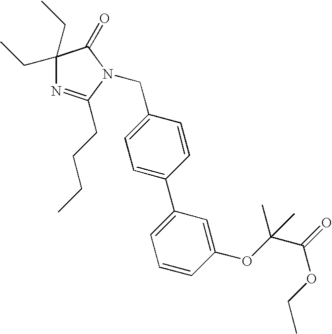

Prepared following the general procedure previously described (Method 14C) using 2-butyl-1-[(3′-hydroxybiphenyl-4-yl)methyl]-4,4-diethyl-1H-imidazol-5(4H)one

(example 13.3) and ethyl 2-bromobutanoate. The product was chromatographed over silica gel (elution gradient cyclohexane/ethyl acetate 9/1 to 7/3). The product was obtained as a colorless oil.

Yield: 70.7%

Rf (cyclohexane/ethyl acetate 60/40): 0.4

IR: νCO 1728 cm⁻¹

NMR ¹H (CDCl₃): 0.73 (t, 6H, J=7.6 Hz); 0.88 (t, 3H, J=7.3 Hz); 1.24 (t, 3H, J=7 Hz2); 1.36 (m, 2H); 1.63 (m, 8H); 1.83 (q, 4H, J=7.3 Hz); 2.41 (t, 2H, J=7.9 Hz); 4.24 (q, 2H, J=7 Hz); 4.71 (s, 2H); 6.81 (dd, 1H, J=7.9 Hz, J=1.8 Hz); 7.09 (m, 1H); 7.26 (m, 4H); 7.52 (d, 2H, J=8.2 Hz6).

### Example 14.15.

### 2-butyl-1-[(3′-((1-ethoxycarbonyl-1-methylmethyl)oxy)-biphenyl-4-yl)methyl]-4,4-diethyl-1H-imidazol-5(4H)one

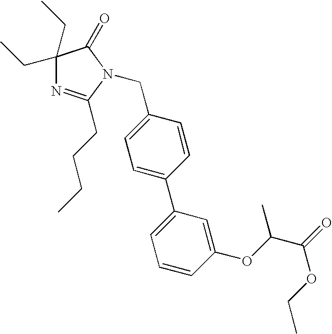

Prepared following the general procedure previously described (Method 14C) using 2-butyl-1-[(3′-hydroxybiphenyl-4-yl)methyl]-4,4-diethyl-1H-imidazol-5(4H)one (example 13.3) and ethyl 2-bromopropanoate. The product was chromatographed over silica gel (elution gradient cyclohexane/ethyl acetate 9/1 to 7/3). The product was obtained as a colorless oil.

Yield: 79.7%

Rf (cyclohexane/ethyl acetate 60/40): 0.4

IR: νCO 1725 and 1755 cm⁻¹

NMR ¹H (CDCl₃): 0.73 (t, 6H, J=7.6 Hz); 0.88 (t, 3H, J=7.3 Hz); 1.24 (t, 3H, J=7 Hz); 1.36 (m, 2H); 1.16 (d, 5H); 1.83 (q, 4H, J=7.3 Hz); 2.41 (t, 2H, J=7.9 Hz); 4.24 (q, 2H, J=7.02); 4.71 (s, 2H); 4.8 (q, 1H, J=7 Hz); 6.83 (dd, 1H, J=7.9 Hz, J=1.8 Hz); 7.1 (m, 1H); 7.17 (m, 1H); 7.3 (m, 3H); 7.52 (d, 2H, J=8.2 Hz).

### Example 14.16.

### 2-butyl-1-[(3′-((1-ethoxycarbonyl-1-spirocyclobutyl-methyl)oxy)-biphenyl-4-yl)methyl]-4-spirocyclohexyl-1H-imidazol-5(4H)-one

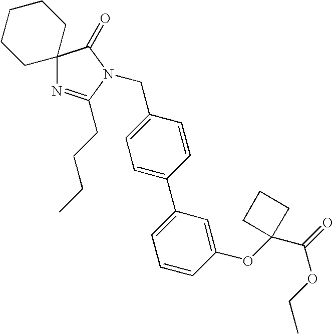

Prepared following the general procedure previously described (Method 14C) using 2-butyl-1-[(3′-hydroxybiphenyl-4-yl)methyl]-4-spirocyclohexyl-1H-imidazol-5(4H)-one (example 13.1) and ethyl 2-bromocyclobutanecarboxylate. The product was chromatographed over silica gel (elution gradient cyclohexane/ethyl acetate 9/1 to 7/3). The product was obtained as a colorless oil.

Yield: 40%

Rf (cyclohexane/ethyl acetate 60/40): 0.5

IR: νCO 1725 cm⁻¹

NMR ¹H (CDCl₃): 0.88 (t, 3H, J=7 Hz); 1.18 (t, 3H, J=7 Hz); 1.35 (m, 2H); 1.71 (m, 12H); 2.02 (m, 2H); 2.5 (m, 4H); 2.78 (m, 2H); 4.22 (q, 2H, J=7 Hz); 4.74 (s, 2H); 6.65 (dd, 1H, J=8.2 Hz, J=2 Hz); 6.94 (m, 1H); 7.18 (m, 3H); 7.3 (m, 1H); 7.51 (d, 2H, J=8.2 Hz9).

### Example 14.17.

### 2-butyl-1-[(3′-((1-ethoxycarbonyl-1-(1,1-dimethylmethyl)-methyl)oxy)-6′-fluoro-biphenyl-4-yl)methyl]-4,4-diethyl-1H-imidazol-5(4H)one

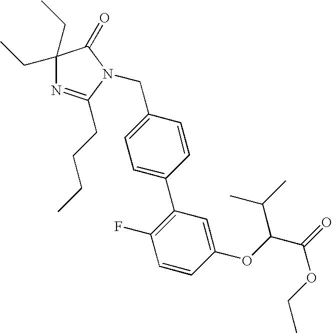

Prepared following the general procedure previously described (Method 14C) using 2-butyl-1-[(6′-fluoro-3′-hydroxybiphenyl-4-yl)methyl]-4,4-diethyl-1H-imidazol-5(4H)one (example 13.4) and ethyl 2-bromoisovalerate. The product was chromatographed over silica gel (elution gradient cyclohexane/ethyl acetate 9/1 to 7/3). The product was obtained as a colorless oil.

Yield: 84.7%

Rf (cyclohexane/ethyl acetate 60/40): 0.3

IR: νCO 1726 and 1750 cm⁻¹

NMR ¹H (CDCl₃): 0.75 (t, 6H, J=7.3 Hz); 0.9 (t, 3H, J=7.3 Hz); 1.1 (t, 6H, J=6.4 Hz); 1.26 (t, 3H, J=7.3 Hz); 1.38 (m, 2H); 1.64 (m, 2H); 1.85 (q, 4H, J=7.3 Hz); 2.29 (m, 1H); 2.43 (t, 2H, J=7.6 Hz); 4.23 (q, 2H, J=7.3 Hz); 4.35 (d, 1H, J=5.3 Hz); 4.73 (s, 2H); 6.82 (m, 1H); 6.95 (m, 1H); 7.05 (t, 1H, J=9.4 Hz); 7.29 (d, 2H, J=7.6 Hz); 7.51 (d, 2H, J=7 Hz).

### Example 14.18.

### 2-butyl-1-[(3′-((1-ethoxycarbonyl-1,1-dimethylmethyl)oxy)-6′-fluoro-biphenyl-4-yl)methyl]-4,4-diethyl-1H-imidazol-5(4H)one

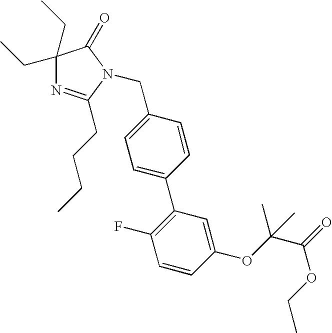

Prepared following the general procedure previously described (Method 14B) using 2-butyl-1-[(6′-fluoro-3′-hydroxybiphenyl-4-yl )methyl]-4,4-diethyl-1H-imidazol-5(4H)one (example 13.4) and ethyl 2-bromoisobutyrate. The product was chromatographed over silica gel (elution gradient cyclohexane/ethyl acetate 9/1 to 7/3). The product was obtained as a colorless oil.

Yield: 42.7%

Rf (cyclohexane/ethyl acetate 60/40): 0.25

IR: νCO 1727 cm⁻¹

NMR ¹H (CDCl₃): 0.73 (t, 6H, J=7.3 Hz); 0.88 (t, 3H, J=7.3 Hz); 1.25 (t, 3H, J=7 Hz); 1.34 (m, 2H); 1.62 (m, 8H); 1.83 (q, 4H, J=7.3 Hz); 2.41 (t, 2H, J=8.2 Hz); 4.23 (q, 2H, J=7 Hz); 4.71 (s, 2H); 6.81 (m, 1H); 6.98 (m, 2H); 7.28 (d, 2H, J=8.2 Hz); 7.48 (d, 2H, J=7.3 Hz).

### Example 14.19.

### 2-butyl-1-[(3′-((1-ethoxycarbonyl-1-methylmethyl)oxy)-2-methyl-biphenyl-4-yl)methyl]-4-spirocyclohexyl-1H-imidazol-5(4H)one

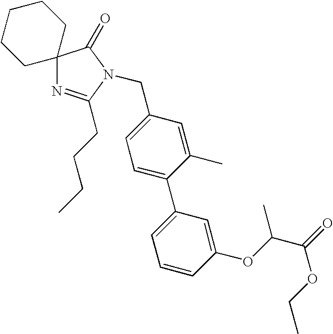

Prepared following the general procedure previously described (Method 14B) using 2-butyl-1-[(3′-hydroxy-2-methyl-biphenyl-4-yl)methyl]-4-spirocyclohexyl-1H-imidazol-5(4H)one (example 13.5) and ethyl 2-bromopropanoate. The product was chromatographed over silica gel (elution gradient cyclohexane/ethyl acetate 9/1 to 7/3). The product was obtained as a colorless oil.

Yield: 77.9%

Rf (cyclohexane/ethyl acetate 60/40): 0.5

IR: νCO 1725 cm⁻¹

NMR ¹H (CDCl₃): 0.89 (t, 3H, J=7.3 Hz); 1.25 (t, 3H, J=7.3 Hz); 1.36 (m, 2H); 1.65 (m, 15H); 2.23 (s, 3H); 2.44 (m, 2H); 4.22 (q, 2H, J=7 Hz); 4.69 (s, 2H); 4.77 (q,1H, J=6.7 Hz); 6.81 (m, 1H); 6.88 (m, 2H); 7.01 (m, 2H); 7.17 (d, 1H, J=7.9 Hz); 7.31 (t, 1H, J=7.9 Hz).

### Example 14.20.

### 2-butyl-1-[(3′-((1-ethoxycarbonyl-1-ethyl methyl)oxy)-2-methyl-biphenyl-4-yl)methyl]-4-spirocyclohexyl-1H-imidazol-5(4H)one

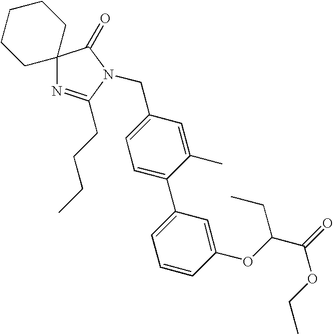

Prepared following the general procedure previously described (Method 14B) using 2-butyl-1-[(3′-hydroxy-2-methyl-biphenyl-4-yl)methyl]-4-spirocyclohexyl-1H-imidazol-5(4H)one (example 13.5) and ethyl 2-bromobutanoate. The product was chromatographed over silica gel (elution gradient cyclohexane/ethyl acetate 9/1 to 7/3). The product was obtained as a colorless oil.

Yield: 77.9%

Rf (cyclohexane/ethyl acetate 60/40): 0.5

IR: νCO 1725 cm⁻¹

NMR ¹H (CDCl₃): 0.89 (t, 3H, J=7.3 Hz); 1.25 (t, 3H, J=7.3 Hz); 1.36 (m, 2H); 1.65 (m, 15H); 2.23 (s, 3H); 2.44 (m, 2H); 4.22 (q, 2H, J=7 Hz); 4.69 (s, 2H); 4.77 (q, 1H, J=6.7 Hz); 6.81 (m, 1H); 6.88 (m, 2H); 7.01 (m, 2H); 7.17 (d, 1H, J=7.9 Hz); 7.31 (t, 1H, J=7.9 Hz).

### Example 14.21.

### 2-butyl-1-[(3′-((1-ethoxycarbonyl-1,1-dimethylmethyl)oxy)-2-methyl-biphenyl-4-yl)methyl]-4-spirocyclohexyl-1H-imidazol-5(4H)one

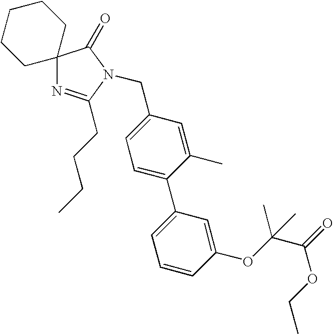

Prepared following the general procedure previously described (Method 14B) using 2-butyl-1-[(3′-hydroxy-2-methylbiphenyl-4-yl)methyl]-4-spirocyclopentyl-1H-imidazol-5(4H)-one (example 13.5) and ethyl 2-bromoisobutyrate. The product was chromatographed over silica gel (elution gradient cyclohexane/ethyl acetate 100/0 to 70/30). The product was obtained as a colorless oil.

Yield: 72.4%

Rf (cyclohexane/ethyl acetate 70/30): 0.55

IR: νCO 1727 cm⁻¹

NMR ¹H (CDCl₃): 0.89 (t, 3h, J=7.3 Hz); 1.24 (t, 3h, J=7 Hz); 1.36 (m, 2H); 1.65 (m, 18H); 2.23 (s, 3H); 2.42 (m, 2H); 4.23 (q, 2H, J=7 Hz); 4.69 (s, 2H); 6.79 (m, 1H); 6.83 (m,1H); 6.91 (m,1H); 7 (m, 2H); 7.15 (d,1H, J=7.6 Hz); 7.27 (m, 1H).

### Example 14.22.

### 2-butyl-1-[(3′-((1-ethoxycarbonyl-1-(1,1-dimethylmethyl)-methyl)oxy)-2-methyl-biphenyl-4-yl)methyl]-4-spirocyclohexyl-1H-imidazol-5(4H)one

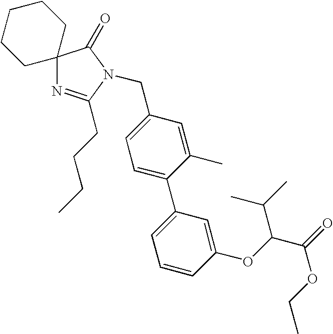

Prepared following the general procedure previously described (Method 14B) using 2-butyl-1-[(3′-hydroxy-2-methylbiphenyl-4-yl)methyl]-4-spirocyclopentyl-1H-imidazol-5(4H)-one (example 13.5) and ethyl 2-bromo-3-methylpropanoate. The product was chromatographed over silica gel (eluent cyclohexane/ethyl acetate 60/40). The product was obtained as a colorless oil.

Yield: 65.1%

Rf (cyclohexane/ethyl acetate 60/40):0.65

IR: νCO 1726 and 1752 cm⁻¹

NMR ¹H (CDCl₃): 0.89 (t, 3H, J=7.3 Hz); 1.09 (t, 6H, J=7 Hz); 1.25 (t, 3H, J=7 Hz); 1.36 (m, 2H); 1.65 (m, 12H); 2.23 (s, 3H); 2.29 (m, 1H); 2.4 (t, 2H, J=7.3 Hz); 4.22 (q, 2H, J=7 Hz); 4.38 (d, 1H, J=5.6 Hz); 4.68 (s, 2H); 6.86 (m, 3H); 7.0 (m, 2H); 7.16 (d,1H, J=7.6 Hz); 7.3 (m, 1H).

### Example 14.23.

### 2-butyl-1-[(3′-((1-ethoxycarbonyl-1,1-dimethylmethyl)-oxy)biphenyl-3-yl)methyl]-4-spirocyclopentyl-1H-imidazol-5(4H)-one

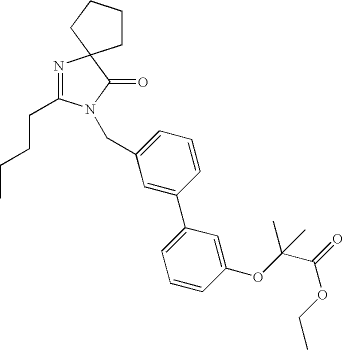

Prepared following the general procedure previously described (Method 14A) using 2-butyl-1-[(3′-hydroxybiphenyl-3-yl )methyl]-4-spirocyclopentyl-1H-imidazol-5(4H)-one (example 13.6) and ethyl 2-bromoisobutyrate. The product was chromatographed over silica gel (eluent petroleum ether/ethyl acetate 60/40). The product was obtained as a colorless oil.

Yield: 36%

Rf (cyclohexane/ethyl acetate 60/40): 0.49

IR: νCO 1727 cm⁻¹

NMR ¹H (CDCl₃): 0.84 (t, 3H, J=7.2 Hz); 1.25 (t, 3H, J=7.1 Hz); 1.29 (m, 2H); 1.57 (m, 2H); 1.63 (s, 6H); 1.80-2.04 (m, 8H); 2.33 (t, 2H, J=7.5 Hz); 4.25 (q, 2H, J=7 Hz); 4.74 (s, 2H); 6.81 (ddd, 1H, J=8 Hz? J=2.4 Hz, J=0.8 Hz); 7.08 (d, 1H, J=2 Hz); 7.13 (dt, 1H, J=7.7 Hz); 7.16 (dt, 1H, J=7.8 Hz, J=1Hz); 7.28 (d, 1H, J=7.9 Hz); 7.33 (d,1H, J=1.8 Hz); 7.38 (t,1H, J=7.5 Hz); 7.47 (dt, 1H, J=7.8 Hz).

### Example 14.24.

### 2-butyl-1-[(2′-((1-ethoxycarbonyl-1,1-dimethylmethyl)-oxy)biphenyl-3-yl)methyl]-4-spirocyclopentyl-1H-imidazol-5(4H)-one

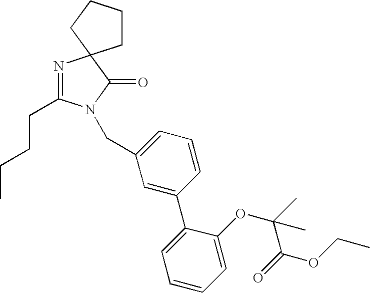

Prepared following the general procedure previously described (Method 14A) using 2-butyl-1-[(2′-hydroxybiphenyl-3-yl)methyl]-4-spirocyclopentyl-1H-imidazol-5(4H)-one (example 13.7) and ethyl 2-bromoisobutyrate. The product was chromatographed over silica gel (eluent petroleum ether/ethyl acetate 60/40). The product was obtained as a colorless oil.

Yield: 46%

Rf (cyclohexane/ethyl acetate 60/40): 0.55

IR: νCO 1731 cm⁻¹

NMR ¹H (CDCl₃): 0.85 (t, 3H, J=7.3 Hz); 1.24 (t, 3H, J=7.1 Hz); 1.29 (m, 2H); 1.41 (s, 6H); 1.57 (m, 2H); 1.75-2.10 (m, 8H); 2.33 (t, 2H, J=7.5 Hz); 4.22 (q, 2H, J=7.1 Hz); 4.73 (s, 2H); 6.86 (dd, 1H, J=8.1 Hz, J=0.8 Hz); 7.01-7.12 (m, 2H); 7.19 (dd, 1H, J=7.8 Hz, J=1.5 Hz); 7.26-7.30 (m, 1H); 7.33-7.39 (m, 2H); 7.47 (d, 1H, J=7.8 Hz).

### Example 14.25.

### 2-butyl-1-[(3′-((1-ethoxycarbonyl-1,1-dimethylmethyl)oxy)-6′-propyl-biphenyl-4-yl)methyl]-4-spirocyclopentyl-1H-imidazol-5(4H)-one

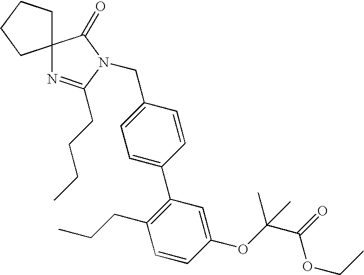

Prepared following the general procedure previously described (Method 14A) using 2-butyl-1-[(3′-hydroxy-6′-propylbiphenyl-4-yl)methyl]-4-spirocyclopentyl-1H-imidazol-5(4H)-one (example 13.8) and ethyl 2-bromoisobutyrate. The product was chromatographed over silica gel (elution gradient petroleum ether/ethyl acetate 80/20 to 60/40). The product was obtained as a yellow oil.

Yield: 78%

Rf (petroleum ether/ethyl acetate 20/80): 0.5

IR: νCO 1633 and 1724 cm⁻¹

NMR ¹H (CDCl₃): 0.79 (t, 3H, J=7.3 Hz); 0.88 (t, 3H, J=7.3 Hz); 1.24 (t, 3H, J=7.1 Hz);1.31-1.49 (m, 4H);1.54-1.67 (m, 2H); 1.61 (s, 6H);1.84-2.08 (m, 8H); 2.34-2.40 (m, 2H);2.43-2.49 (m, 2H); 4.24 (q, 2H, J=7.1 Hz); 4.76 (s, 2H); 6.71 (d, 1H, J=2.6 Hz); 6.8 (dd, 1H, J=8.3 Hz, J=2.6 Hz);7.13-7.33 (m, 5H).

### Example 14.26.

### 2-butyl-1-[(4′-((1-ethoxycarbonyl-1,1-dimethylmethyl)-oxy)biphenyl-3-yl)methyl]-4-spirocyclopentyl-1H-imidazol-5(4H)-one

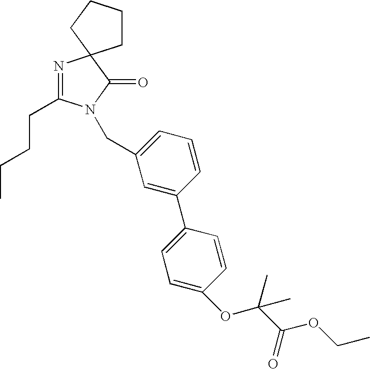

Prepared following the general procedure previously described (Method 14A) using 2-butyl-1-[(4′-hydroxybiphenyl-3-yl)methyl]-4-spirocyclopentyl-1H-imidazol-5(4H)-one (example 13.9) and ethyl 2-bromoisobutyrate. The product was chromatographed over silica gel (elution gradient petroleum ether/ethyl acetate 80/20 to 60/40). The product was obtained as a yellow oil.

Yield: 85%

Rf (petroleum ether/ethyl acetate 40/60): 0.3

IR: νCO 1631 and 1728 cm⁻¹

NMR ¹H (CDCl₃): 0.84 (t, 3H, J=7.3 Hz); 1.18-1.38 (m, 5H); 1.51-1.63 (m, 8H); 1.80-2.05 (m, 8H); 2.33 (m, 2H); 4.25 (q, 2H, J=7.1 Hz); 4.73 (s, 2H); 6.91 (d, 2H, J=8.7 Hz); 7.08 (d,1H, J=7.5 Hz); 7.32-7.47 (m, 5H).

### Example 14.27.

### 2-butyl-1-[(3′-((1-ethoxycarbonyl-1,1-dimethylmethyl)oxy)-2′-fluoro-biphenyl-4-yl)methyl]-4-spirocyclopentyl-1H-imidazol-5(4H)-one

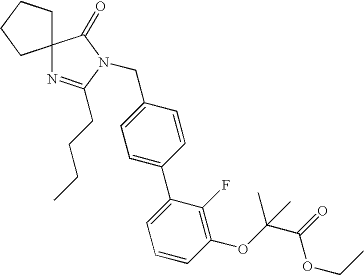

Prepared following the general procedure previously described (Method 14A) using 2-butyl-1-[(2′-fluoro-3′-hydroxybiphenyl-4-yl)methyl]-4-spirocyclopentyl-1H-imidazol-5(4H)-one (example 13.10) and ethyl 2-bromoisobutyrate. The product was chromatographed over silica gel (elution gradient petroleum ether/ethyl acetate 80/20 to 60/40). The product was obtained as a yellow oil.

Yield: 83%

Rf (petroleum ether/ethyl acetate 60/40): 0.3

IR: νCO 1632 and 1725 cm⁻¹

NMR ¹H (CDCl₃): 0.87 (t, 3H, J=7.3 Hz); 1.29 (t, 3H, J=7.1 Hz); 1.29-1.38 (m, 2H); 1.54-1.61 (m, 2H); 1.61 (s, 6H); 1.82-2.04 (m, 8H); 2.32-2.36 (m, 2H); 4.26 (q, 2H, J=7.1 Hz); 4.73 (s, 2H); 6.94-6.98 (m, 1H); 7.03-7.07 (m, 2H); 7.23 (d, 2H, J=8.2 Hz); 7.50 (d, 2H, J=8 Hz).

### Example 14.28.

### 2-butyl-1-[(3′-((1-ethoxycarbonyl-1,1-dimethylmethyl)oxy)-4′-methoxy-biphenyl-4-yl)methyl]-4-spirocyclopentyl-1H-imidazol-5(4H)-one

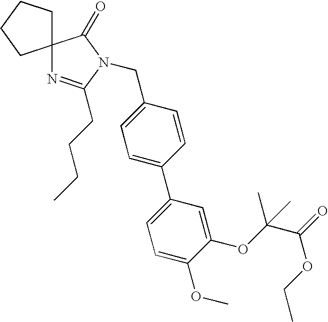

Prepared following the general procedure previously described (Method 14A) using 2-butyl-1-[(3′-hydroxy-4′-methoxybiphenyl-4-yl)methyl]-4-spirocyclopentyl-1H-imidazol-5(4H)-one (example 13.11) and ethyl 2-bromoisobutyrate. The product was chromatographed over silica gel (elution gradient petroleum ether/ethyl acetate 80/20 to 60/40). The product was obtained as a yellow oil.

Yield: 51%

Rf (petroleum ether/ethyl acetate 60/40): 0.37

IR: νCO 1627 and 1732 cm⁻¹

NMR ¹H (CDCl₃): 0.84 (t, 3H, J=7.3 Hz); 1.24 (t, 3H, J=7.1 Hz); 1.28-1.36 (m, 2H); 1.52-1.60 (m, 2H); 1.58 (s, 6H); 1.79-2.02 (m, 8H); 2.29-2.33 (m, 2H); 3.82 (s, 3H); 4.23 (q, 2H, J=7.1 Hz); 4.68 (s, 2H); 6.91 (d, 1H, J=8.4 Hz); 7.14 (d, 1H, J=2.2 Hz); 7.16-7.21 (m, 3H); 7.45 (d, 2H, J=8.2 Hz).

### Example 14.29.

### 2-butyl-1-[(3′-((1-ethoxycarbonyl-1,1-dimethylmethyl)oxy)-6′-ethyl-biphenyl-4-yl)methyl]-4-spirocyclopentyl-1H-imidazol-5(4H)-one

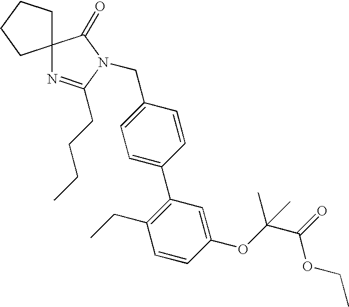

Prepared following the general procedure previously described (Method 14A) using 2-butyl-1-[(6′-ethyl-3′-hydroxybiphenyl-4-yl)methyl]-4-spirocyclopentyl-1H-imidazol-5(4H)-one (example 13.12) and ethyl 2-bromoisobutyrate. The product was chromatographed over silica gel (elution gradient petroleum ether/ethyl acetate 80/20 to 70/30). The product was obtained as a yellow oil.

Yield: 95%

Rf (petroleum ether/ethyl acetate 60/40): 0.2

IR: νCO 1633 and 1727 cm⁻¹

NMR ¹H (CDCl₃): 0.86 (t, 3H, J=7.3 Hz); 1.04 (t, 3H, J=7.5 Hz); 1.24 (t, 3H, J=7.1 Hz); 1.26-1.40 (m, 2H); 1.51-1.63 (m, 8H); 1.81-2.03 (m, 8H); 2.31-2.37 (m, 2H); 2.48 (q, 2H, J=7.5 Hz); 4.22 (q, 2H, J=7.1 Hz); 4.72 (s, 2H); 6.89 (d, 1H, J=2.6 Hz); 6.98 (dd, 1H, J=8.4 Hz, J=2.6 Hz); 7.12-7.17 (m, 3H); 7.23 (d, 2H, J=8.4 Hz).

### Example 14.30.

### 2-butyl-1-[(3′-((1-ethoxycarbonyl-1,1-dimethylmethyl)oxy)-4′-isobutyl-biphenyl-4-yl)methyl]-4-spirocyclopentyl-1H-imidazol-5(4H)-one

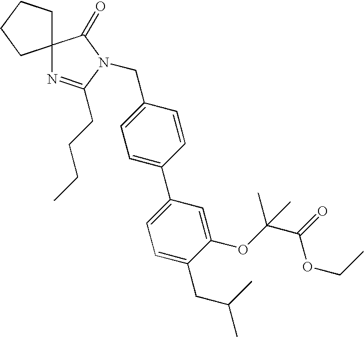

Prepared following the general procedure previously described (Method 14A) using 2-butyl-1-[(3′-hydroxy-4′-isobutylbiphenyl-4-yl)methyl]-4-spirocyclopentyl-1H-imidazol-5(4H)-one (example 13.13) and ethyl 2-bromoisobutyrate. The product was chromatographed over silica gel (elution gradient petroleum ether/ethyl acetate 80/20 to 60/40). The product was obtained as a yellow oil.

Yield: 50%

Rf (petroleum ether/ethyl acetate 60/40): 0.3

IR: νCO 1632 and 1727 cm⁻¹

NMR ¹H (CDCl₃): 0.85 (t, 3H, J=7.3 Hz); 0.92 (d, 6H, J=6.6 Hz); 1.17 (t, 3H, J=7.1 Hz); 1.28-1.31 (m, 2H); 1.53-1.60 (m, 3H); 1.64 (s, 6H); 1.80-2.05 (m, 8H); 2.30-2.34 (m, 2H); 2.51 (d, 2H, J=7.1 Hz); 4.21 (q, 2H, J=7.1 Hz); 4.70 (s, 2H); 6.85 (dd, 1H, J=1.5 Hz); 7.08 (d, 1H, J=7.7 Hz, J=1.5 Hz); 7.14 (d, 1H, J=7.7 Hz); 7.18 (d, 2H, J=8.2 Hz); 7.46 (d, 2H, J=8.2 Hz).

### Example 14.31.

### 2-butyl-1-[(3′-((1-ethoxycarbonyl-1.1-dimethylmethyl)oxy)-3-methoxy-biphenyl-4-yl )methyl]-4-spirocyclopentyl-1H-imidazol-5(4H)-one

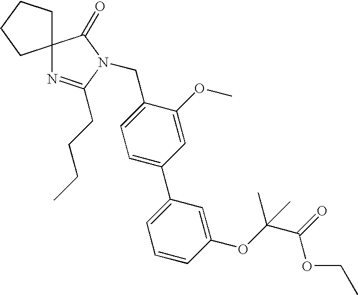

Prepared following the general procedure previously described (Method 14A) using 2-butyl-1-[(3′-hydroxy-3-methoxybiphenyl-4-yl )methyl]-4-spirocyclopentyl-1H-imidazol-5(4H)-one (example 13.14) and ethyl 2-bromoisobutyrate. The product was chromatographed over silica gel (eluent petroleum ether/ethyl acetate 80/20). The product was obtained as a colorless oil.

Yield: 45%

Rf (petroleum ether/ethyl acetate 70/30): 0.57

IR: νCO 1630 and 1728 cm⁻¹

NMR ¹H (CDCl₃): 0.89 (t, 3H, J=7.5 Hz); 1.27 (t, 3H, J=7.5 Hz); 1.31-1.40 (m, 2H); 1.54-1.61 (m, 2H); 1.66 (s, 6H); 1.82-2.10 (m, 8H); 2.34-2.41 (m, 2H); 3.93 (s, 3H); 4.28 (q, 2H, J=7.5 Hz); 4.74 (s, 2H); 6.83 (dd, 1H, J=7.5 Hz, J=2.5 Hz); 7.02-7.12 (m, 4H); 7.22 (d, 1H, J=7.5 Hz); 7.34 (d, 1H, J=7.5 Hz).

### Example 14.32.

### 2-butyl-1-[(3′-((1-ethoxycarbonyl-1-methylmethyl)oxy)-6′-propyl-biphenyl-4-yl)methyl]-4-spirocyclopentyl-1H-imidazol-5(4H)-one

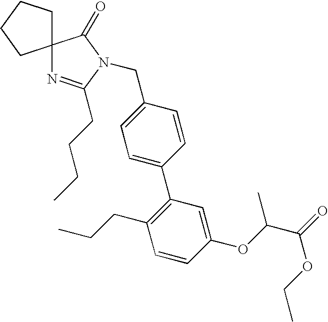

Prepared following the general procedure previously described (Method 14B) using 2-butyl-1-[(3′-hydroxy-6′-propylbiphenyl-4-yl)methyl]-4-spirocyclopentyl-1H-imidazol-5(4H)-one (example 13.8) and ethyl 2-bromopropanoate. The product was chromatographed over silica gel (elution gradient cyclohexane/ethyl acetate 100/0 to 70/30). The product was obtained as a colorless oil.

Yield: 84%

Rf (cyclohexane/ethyl acetate 70/30): 0.2

IR: νCO 1726 and 1632 cm⁻¹

NMR ¹H (CDCl₃): 0.78 (t, 3H, J=7.3 Hz); 0.87 (t, 3H, J=7.3 Hz); 1.24 (t, 3H, J=7 Hz); 1.35 (m, 4H); 1.62 (m, 5H); 1.87 (m, 2H); 2.01 (m, 6H); 2.40 (m, 4H); 4.22 (q, 2H, J=7.3 Hz); 4.73 (m, 3H); 6.71 (d, 1H, J=2.9 Hz); 6.81 (dd, 1H, J=8.5 Hz, J=2.9 Hz); 7.16 (m, 3H); 7.24 (d, 2H, J=8.2 Hz).

### Example 14.33.

### 2-butyl-1-[(3′-((1-ethoxycarbonylmethyl)oxy)-6′-propyl-biphenyl-4-yl)methyl]-4-spirocyclopentyl-1H-imidazol-5(4H)-one

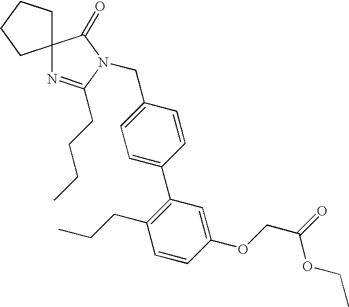

Prepared following the general procedure previously described (Method 14B) using 2-butyl-1-[(3′-hydroxy-6′-propylbiphenyl-4-yl)methyl]-4-spirocyclopentyl-1H-imidazol-5(4H)-one (example 13.8) and ethyl 2-bromoacetate. The product was chromatographed over silica gel (elution gradient cyclohexane/ethyl acetate 100/0 to 70/30). The product was obtained as a colorless oil.

Yield: 46%

Rf (cyclohexane/ethyl acetate 70/30): 0.2

IR: νCO 1724,1759 and 1632 cm⁻¹

NMR ¹H (CDCl₃): 0.78 (t, 3H, J=7.3 Hz); 0.88 (t, 3H, J=7.3 Hz); 1.29 (t, 3H, J=7.2 Hz); 1.39 (m, 6H); 1.59 (m, 2H); 1.98 (m, 6H); 2.45 (m, 4H); 4.27 (q, 2H, J=7.2 Hz); 4.61 (s, 2H); 4.77 (s, 2H); 6.73 (d, 1H, J=2.9 Hz); 6.86 (dd, 1H, J=8.5 Hz, J=2.9 Hz); 7.18 (m, 3H); 7.26 (m, 2H).

### Example 14.34.

### 2-butyl-1-[(3′-((1-ethoxycarbonyl-1-ethylmethyl)oxy)-6′-propyl-biphenyl-4-yl)methyl]-4-spirocyclopentyl-1H-imidazol-5(4H)-one

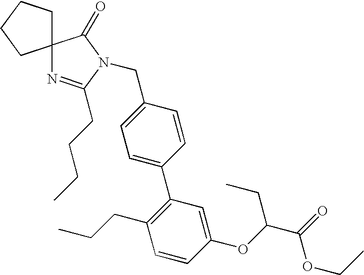

Prepared following the general procedure previously described (Method 14B) using 2-butyl-1-[(3′-hydroxy-6′-propylbiphenyl-4-yl)methyl]-4-spirocyclopentyl-1H-imidazol-5(4H)-one (example 13.8) and ethyl 2-bromobutanoate. The product was chromatographed over silica gel (elution gradient cyclohexane/ethyl acetate 100/0 to 70/30). The product was obtained as a colorless oil.

Yield: 90%

Rf (cyclohexane/ethyl acetate 70/30): 0.2

IR: νCO 1726,1753 and 1633 cm⁻¹

NMR ¹H (CDCl₃): 0.78 (t, 3H, J=7.3 Hz); 0.88 (t, 3H, J=7 Hz); 1.08 (t, 3H, J=7.3 Hz); 1.25 (t, 3H, J=7.3 Hz); 1.36 (m, 4H); 1.63 (m, 2H); 1.89-2.05 (m, 10H); 2.44 (m, 4H); 4.22 (q, 2H, J=7 Hz); 4.54 (t, 1H, J=6.1 Hz); 4.76 (s, 2H); 6.72 (d, 1H, J=2.9 Hz); 6.81 (dd, 1H, J=8.5 Hz, J=2.9 Hz); 7.16 (m, 3H); 7.24 (m, 2H).

### Example 14.35.

### 2-butyl-1-[(3′-((1-ethoxycarbonyl-1-(1,1-dimethylmethyl)-methyl)oxy)-6′-propyl-biphenyl-4-yl)methyl]-4-spirocyclopentyl-1H-imidazol-5(4H)-one

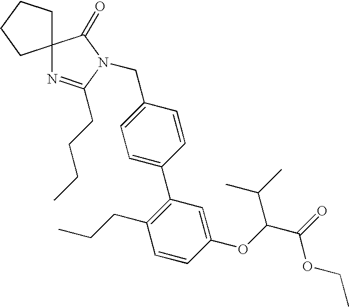

Prepared following the general procedure previously described (Method 14B) using 2-butyl-1-[(3′-hydroxy-6′-propylbiphenyl-4-yl)methyl]-4-spirocyclopentyl-1H-imidazol-5(4H)-one (example 13.8) and ethyl 2-bromo-3-methylbutanoate. The product was chromatographed over silica gel (elution gradient cyclohexane/ethyl acetate 100/0 to 70/30). The product was obtained as a colorless oil.

Yield: 73%

Rf (cyclohexane/ethyl acetate 70/30): 0.2

IR: νCO 1727 and 1632 cm⁻¹

NMR ¹H (CDCl₃): 0.78 (t, 3H, J=7.3 Hz); 0.88 (t, 3H, J=7.3 Hz); 1.06 (m, 6H); 1.24 (t, 3H, J=7 Hz); 1.38 (m, 4H); 1.60 (m, 2H); 1.97 (m, 8H); 2.24 (m, 1H); 2.43 (m, 4H); 4.21 (q, 2H, J=7.3 Hz); 4.34 (d, 1H, J=5.6 Hz); 4.77 (s, 2H); 6.72 (d, 1H, J=2.9 Hz); 6.81 (dd, 1H, J=8.5 Hz, J=2.9 Hz); 7.16 (m, 3H); 7.26 (m, 2H).

### Example 14.36.

### 2-butyl-1-[(3′-((1-ethoxycarbonyl-1,1-dimethylmethyl)oxy)-6′-isobutyl-biphenyl-4-yl)methyl]-4-spirocyclopentyl-1H-imidazol-5(4H)-one

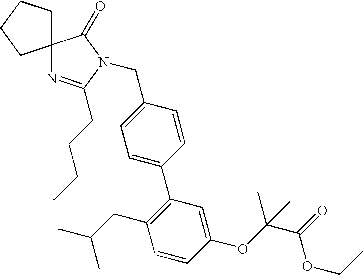

Prepared following the general procedure previously described (Method 14A) using 2-butyl-1-[(3′-hydroxy-6′-isobutylbiphenyl-4-yl)methyl]-4-spirocyclopentyl-1H-imidazol-5(4H)-one (example 13.15) and ethyl 2-bromoisobutyrate. The product was chromatographed over silica gel (elution gradient petroleum ether/ethyl acetate 80/20 to 60/40). The product was obtained as a colorless oil.

Yield: 91%

Rf (petroleum ether/ethyl acetate 60/40): 0.35

IR: νCO 1633 and 1728 cm⁻¹

NMR ¹H (CDCl₃): 0.69 (d, 6H, J=6.6 Hz); 0.88 (t, 3H, J=7.3 Hz); 1.23 (t, 3H, J=7.1 Hz); 1.30-1.42 (m, 2H); 1.54-1.66 (m, 3H); 1.60 (s, 6H); 1.83-2.06 (m, 8H); 2.31-2.40 (m, 4H); 4.23 (q, 2H, J=7.1 Hz); 4.75 (s, 2H); 6.70 (dd, 1H, J=2.7 Hz); 6.74-6.80 (m, 1H); 7.09 (d, 1H, J=8.4 Hz); 7.16 (d, 2H, J=8.2 Hz); 7.23 (d, 2H, J=8.2 Hz).

### Example 14.37.

### 2-butyl-1-[(3′-((1-ethoxycarbonyl-1,1-dimethylmethyl)oxy)-2-ethyl-biphenyl-4-yl)methyl]-4-spirocyclopentyl-1H-imidazol-5(4H)-one

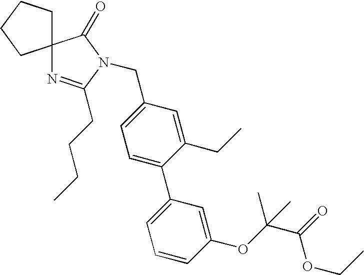

Prepared following the general procedure previously described (Method 14A) using 2-butyl-1-[(3′-hydroxy-2-ethylbiphenyl-4-yl)methyl]-4-spirocyclopentyl-1H-imidazol-5(4H)-one (example 13.16) and ethyl 2-bromoisobutyrate. The product was chromatographed over silica gel (eluent petroleum ether/ethyl acetate 80/20). The product was obtained as a colorless oil.

Yield: 43%

Rf (petroleum ether/ethyl acetate 70/30): 0.63

IR: νCO 1626 and 1720 cm⁻¹

NMR ¹H (CDCl₃): 0.89 (t, 3H, J=7.5 Hz); 1.06 (t, 3H, J=7.5 Hz); 1.25 (t, 3H, J=7.5 Hz); 1.32-1.41 (m, 2H); 1.55-1.67 (m, 8H); 1.83-2.06 (m, 8H); 2.35-2.41 (m, 2H); 2.53 (q, 2H, J=7.5 Hz); 4.24 (q, 2H, J=7.5 Hz); 4.72 (s, 2H); 6.79-6.93 (m, 3H); 7.01 (dd, 1H, J=7.5 Hz, J=2.5 Hz); 7.07 (sl, 1H); 7.14 (d, 1H, J=7.5 Hz); 7.34 (m, 1H).

### Example 14.38.

### 2-butyl-6′-cyano-1-[(3′-((1-ethoxycarbonyl-1,1-dimethylmethyl)oxy)biphenyl-4-yl)methyl]-4-spirocyclopentyl-1H-imidazol-5(4H)-one

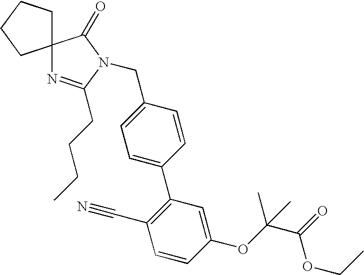

Prepared following the general procedure previously described (Method 14A) using 2-butyl-1-[(6′-cyano-3′-hydroxybiphenyl-4-yl)methyl]-4-spirocyclopentyl-1H-imidazol-5(4H)-one (example 13.17) and ethyl 2-bromoisobutyrate. The product was chromatographed over silica gel (elution gradient petroleum ether/ethyl acetate 80/20 to 50/50). The product was obtained as a yellow oil.

Yield: 12%

Rf (petroleum ether/ethyl acetate 40/60): 0.5

IR: νCO 1632 and 1726 cm⁻¹

NMR ¹H (CDCl₃): 0.87 (t, 3H, J=7.3 Hz); 1.22 (t, 3H, J=7.1 Hz); 1.51-1.67 (m, 2H); 1.67 (s, 8H); 1.82-2.04 (m, 8H); 2.32-2.38 (m, 2H); 4.23 (q, 2H, J=7.1 Hz); 4.74 (s, 2H); 6.81 (dd, 1H, J=8.6 Hz, J=2.5 Hz); 6.90 (d, 1H, J=2.5 Hz); 7.26 (d, 2H, J=8.1 Hz); 7.51 (d, 2H, J=8.1 Hz); 7.62 (d, 1H, J=8.6 Hz).

### Example 14.39.

### 2-butyl-1-[(3′-((1-ethoxycarbonyl-1,1-dimethylmethyl)oxy)-2-methoxy-biphenyl-4-yl )methyl]-4-spirocyclopentyl-1H-imidazol-5(4H)-one

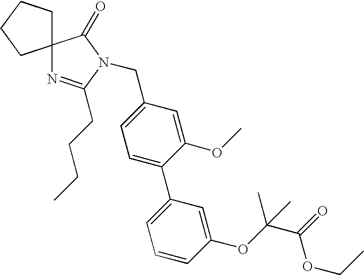

Prepared following the general procedure previously described (Method 14A) using 2-butyl-1-[(3′-hydroxy-2-methoxybiphenyl-4-yl)methyl]-4-spirocyclopentyl-1H-imidazol-5(4H)-one (example 13.18) and ethyl 2-bromoisobutyrate. The product was chromatographed over silica gel (eluent petroleum ether/ethyl acetate 80/20). The product was obtained as a colorless oil.

Yield: 72%

Rf (petroleum ether/ethyl acetate 70/30): 0.55

IR: νCO 1633 and 1725 cm⁻¹

NMR ¹H (CDCl₃): 0.77 (t, 3H, J=7.5 Hz); 1.15-1.20 (m, 2H); 1.26 (t, 3H, J=7.5 Hz); 1.33-1.40 (m, 2H); 1.62 (s, 6H); 1.77-2.01 (m, 10H); 3.76 (s, 3H); 4.24 (q, 2H, J=7.5 Hz); 4.62 (s, 2H); 6.55 (d, 1H, J=7.5 Hz); 6.81 (sl, 1H); 6.83-6.93 (m, 3H); 7.17 (d, 1H, J=7.5 Hz); 7.29 (m, 1H, J=7.5 Hz).

### Example 14.40.

### 2-butyl-1-[(3′-((1-ethoxycarbonyl-1,1-dimethylmethyl)oxy)-3-methyl-biphenyl-4-yl)methyl]-4-spirocyclopentyl-1H-imidazol-5(4H)-one

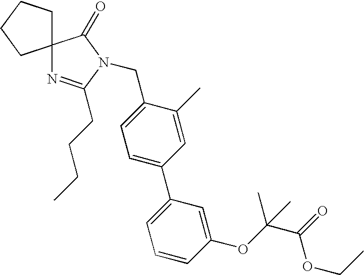

Prepared following the general procedure previously described (Method 14A) using 2-butyl-1-[(3′-hydroxy-3-methylbiphenyl-4-yl)methyl]-4-spirocyclopentyl-1H-imidazol-5(4H)-one (example 13.19) and ethyl 2-bromoisobutyrate. The product was chromatographed over silica gel (eluent petroleum ether/ethyl acetate 80/20). The product was obtained as a colorless oil.

Yield: 70%

Rf (petroleum ether/ethyl acetate 70/30): 0.6

IR: νCO 1623 and 1729 cm⁻¹

NMR ¹H (CDCl₃): 0.85 (t, 3H, J=7.5 Hz); 1.23-1.34 (m, 5H); 1.52-1.60 (m, 2H); 1.63 (s, 6H); 1.48-2.02 (m, 8H); 2.26-2.30 (m, 2H); 2.37 (s, 3H); 4.24 (q, 2H, J=7.5 Hz); 4.70 (s, 2H); 6.81 (dd,1H, J=7.5 Hz, J=2.5 Hz); 6.92 (d,1H, J=7.5 Hz); 7.08 (m, 1H); 7.19 (d, 1H, J=7.5 Hz); 7.26-7.30 (m, 1H); 7.33-7.37 (m, 2H).

### Example 14.41.

### 2-butyl-1-[[2-[(4-(1-ethoxycarbonyl-1,1-dimethylmethyloxy)phenyl]-6-methyl-thiazolo[3,2-b][1,2,4]triazol-5-yl]methyl]-4-spirocyclopentyl-1H-imidazol-5(4H)-one

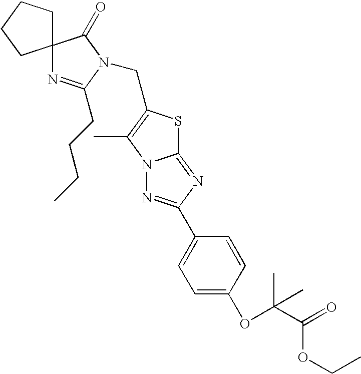

Prepared following the general procedure previously described (Method 14B) using 2-butyl-1-[[2-(4-hydroxyphenyl)-6-methyl-thiazolo[3,2-b][1,2,4]triazol-5-yl]methyl]-4-spirocyclopentyl-1H-imidazol-5(4H)-one (example 13.20) and ethyl 2-bromoisobutyrate. The product was chromatographed over silica gel (elution gradient petroleum ether/ethyl acetate 90/10 to 70/30). The product was obtained as a colorless oil.

Yield: 91.2%

Rf (cyclohexane/ethyl acetate 50/50): 0.3

IR: νCO 1634 and 1731 cm⁻¹

NMR ¹H (CDCl₃): 0.94 (t, 3H, J=7.6 Hz); 1.25 (m, 5H); 1.43 (m, 2H); 1.67 (m, 8H); 1.95 (m, 6H); 2.58 (t, 2H, J=7.6 Hz); 2.64 (s, 3H); 4.24 (q, 2H, J=7.3 Hz); 4.94 (s, 2H); 6.90 (d, 2H, J=8.8 Hz); 7.99 (d, 2H, J=8.8 Hz).

### Example 14.42.

### 2-butyl-1-[[2-[(3-(1-ethoxycarbonyl-1,1-dimethylmethyloxy)phenyl]-6-methyl-thiazolo[3,2-b][1,2,4]triazol-5-yl]methyl]-4-spirocyclopentyl-1H-imidazol-5(4H)-one

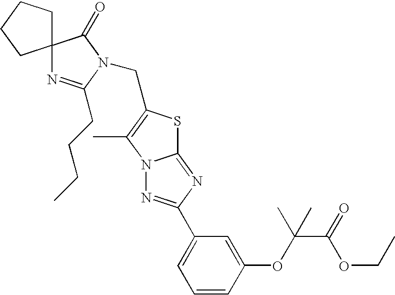

Prepared following the general procedure previously described (Method 14B) using 2-butyl-1-[[2-(3-hydroxyphenyl)-6-methyl-thiazolo[3,2-b][1,2,4]triazol-5-yl]methyl]-4-spirocyclopentyl-1H-imidazol-5(4H)-one (example 13.21) and ethyl 2-bromoisobutyrate. The product was chromatographed over silica gel (elution gradient cyclohexane/ethyl acetate 80/20 to 50/50). The product was obtained as a colorless oil.

Yield: 77.1%

Rf (cyclohexane/ethyl acetate 50/50): 0.3

IR: νCO 1635 and 1731 cm⁻¹

NMR ¹H (CDCl₃): 0.93 (t, 3H, J=7.6 Hz); 1.26 (t, 3H, J=7 Hz); 1.42 (m, 2H); 1.72 (m, 10H); 1.92 (m, 6H); 2.44 (t, 2H, J=7.9 Hz); 2.06 (s, 3H); 4.25 (q, 2H, J=7 Hz); 4.73 (s, 2H); 6.89 (dd, 1H, J=7.9 Hz, J=2.3 Hz); 7.30 (m, 1H); 7.67 (m, 1H); 7.76 (d, 1H, J=7.6 Hz).

### Example 14.43.

### 2-butyl-1-[(3′-((1-ethoxycarbonyl-1,1-dimethylmethyl)oxy)-2-propyl-biphenyl-4-yl)methyl]-4-spirocyclopentyl-1H-imidazol-5(4H)-one

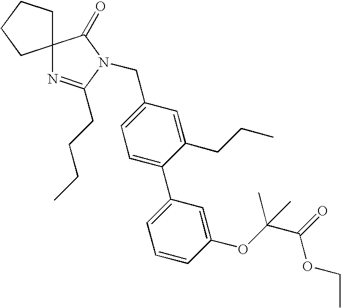

Prepared following the general procedure previously described (Method 14A) using 2-butyl-1-[(3′-hydroxy-2-propylbiphenyl-4-yl)methyl]-4-spirocyclopentyl-1H-imidazol-5(4H)-one (example 13.22) and ethyl 2-bromoisobutyrate. The product was chromatographed over silica gel (eluent petroleum ether/ethyl acetate 80/20). The product was obtained as a colorless oil.

Yield: 72%

Rf (petroleum ether/ethyl acetate 70/30): 0.65

IR: νCO 1628 and 1726 cm⁻¹

NMR ¹H (CDCl₃): 0.77 (t, 3H, J=7.5 Hz); 0.88 (t, 3H, J=7.5 Hz); 1.23 (t, 3H, J=7.5 Hz); 1.30-1.47 (m, 4H); 1.55-1.61 (m, 8H); 1.80-2.01 (m, 8H); 2.34-2.38 (m, 2H); 2.51 (t, 2H, J=7.5 Hz); 4.23 (q, 2H, J=7.5 Hz); 4.70 (s, 2H); 6.77 (m, 1H); 6.84 (dd, 1H, J=7.5 Hz, J=2.5 Hz); 6.89 (d, 1H, J=7.5 Hz); 6.99 (dd, 1H, J=7.5 Hz, J=2.5 Hz); 7.07 (s, 1H); 7.12 (d, 1H, J=7.5 Hz); 7.25 (m, 1H).

### Example 14.44.

### 2-butyl-1-[(3′-((1-ethoxycarbonyl-1,1-dimethylmethyl)oxy)-4′-nitro-biphenyl-4-yl)methyl]-4-spirocyclopentyl-1H-imidazol-5(4H)-one

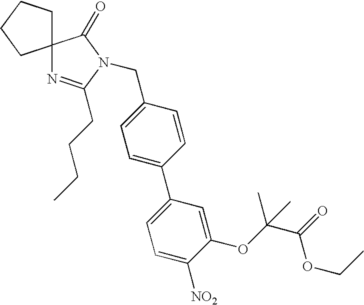

Prepared following the general procedure previously described (Method 14A) using 2-butyl-1-[(3′-hydroxy-4′-nitrobiphenyl-4-yl)methyl]-4-spirocyclopentyl-1H-imidazol-5(4H)-one (example 13.23) and ethyl 2-bromoisobutyrate. The product was chromatographed over silica gel (elution gradient petroleum ether/ethyl acetate 80/20 to 50/50). The product was obtained as a yellow oil.

Yield: 52%

Rf (petroleum ether/ethyl acetate 50/50): 0.5

IR: νCO 1632 and 1726 cm⁻¹

NMR ¹ H (CDCl₃): 0.85 (t, 3H, J=7.3 Hz); 1.21 (t, 3H, J=7.1 Hz); 1.28-1.40 (m, 2H); 1.52-1.64 (m, 2H); 1.67 (s, 6H); 1.79-2.03 (m, 8H); 2.29-2.35 (m, 2H); 4.23 (q, 2H, J=7.1 Hz); 4.72 (s, 2H); 7.15 (d, 1H, J=1.6 Hz); 7.23-7.26 (m, 3H); 4715-7.19 (d, 2H, J=8.2 Hz); 7.84 (d, 1H, J=8.4 Hz).

### Example 14.45.

### 2-butyl-1-[(3′-((1-ethoxycarbonyl-1,1-dimethylmethyl)oxy)-2-trifluoromethylbiphenyl-4-yl)methyl]-4-spirocyclopentyl-1H-imidazol-5(4H)-one

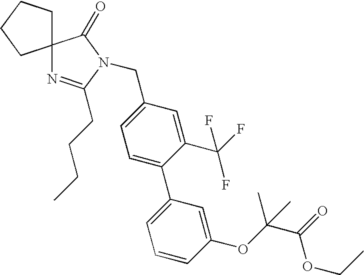

Prepared following the general procedure previously described (Method 14A) using 2-butyl-1-[(3′-hydroxy-2-trifluoromethylbiphenyl-4-yl)methyl]-4-spirocyclopentyl-1H-imidazol-5(4H)-one (example 13.24) and ethyl 2-bromoisobutyrate. The product was chromatographed over silica gel (eluent petroleum ether/ethyl acetate 70/30). The product was obtained as a colorless oil.

Yield: 96%

Rf (eluent petroleum ether/ethyl acetate 60/40): 0.33

IR: νCO 1633 and 1728 cm⁻¹

NMR ¹H (CDCl₃): 0.89 (t, 3H, J=7.5 Hz); 1.23 (t, 3H, J=7.5 Hz); 1.33-1.44 (m, 2H); 1.56-1.68 (m, 8H); 1.84-2.01 (m, 8H); 2.34-2.40 (m, 2H); 2.22 (q, 2H, J=7.5 Hz); 4.78 (s, 2H); 6.81 (s, 1H); 6.92-6.94 (m, 2H); 7.24-7.36 (m, 3H); 7.51 (s, 1H).

### Example 14.46.

### 2-butyl-1-[(3′-((1-ethoxycarbonyl-1,1-dimethyl methyl)oxy)-2-nitro-biphenyl-4-yl)methyl]-4-spirocyclopentyl-1H-imidazol-5(4H)-one

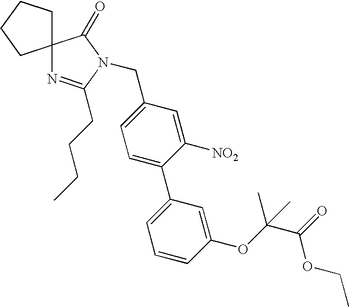

Prepared following the general procedure previously described (Method 14A) using 2-butyl-1-[(3′-hydroxy-2-nitrobiphenyl-4-yl)methyl]-4-spirocyclopentyl-1H-imidazol-5(4H)-one (example 13.25) and ethyl 2-bromoisobutyrate. The product was chromatographed over silica gel (eluent petroleum ether/ethyl acetate 70/30). The product was obtained as a colorless oil.

Yield: 94%

Rf (petroleum ether/ethyl acetate 60/40): 0.33

IR: νCO 1635 and 1726 cm⁻¹

NMR ¹H (CDCl₃): 0.91 (t, 3H, J=7.5 Hz); 1.24 (t, 3H, J=7.5 Hz); 1.34-1.43 (m, 2H); 1.54-1.70 (m, 8H); 1.84-2.04 (m, 8H); 2.34-2.40 (m, 2H); 2.24 (q, 2H, J=7.5 Hz); 4.77 (s, 2H); 6.79 (s, 1H); 6.88-6.93 (m, 2H); 7.29 (m, 1H); 7.41 (s, 2H); 7.64 (s, 1H).

### Example 14.47.

### 2-butyl-1-[(3′-((1-ethoxycarbonyl-1,1-dimethylmethyl)oxy)-4′-propyl-biphenyl-4-yl)methyl]-4-spirocyclopentyl-1H-imidazol-5(4H)-one

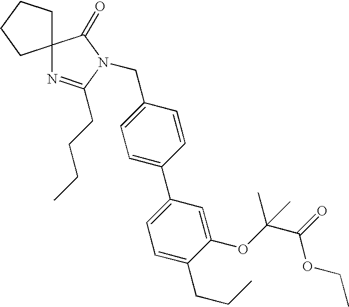

Prepared following the general procedure previously described (Method 14A) using 2-butyl-1-[(3′-hydroxy-4′-propylhylbiphenyl-4-yl)methyl]-4-spirocyclopentyl-1H-imidazol-5(4H)-one (example 13.26) and ethyl 2-bromoisobutyrate. The product was chromatographed over silica gel (elution gradient petroleum ether/ethyl acetate 80/20 to 60/40). The product was obtained as a yellow oil.

Yield: 75%

Rf (petroleum ether/ethyl acetate 50/50): 0.5

IR: νCO 1633 and 1727 cm⁻¹

NMR ¹H (CDCl₃): 0.84 (t, 3H, J=7.3 Hz); 0.95 (t, 3H, J=7.3 Hz); 1.17 (t, 3H, J=7.1 Hz); 1.23-1.38 (m, 2H); 1.53-1.68 (m, 10H); 1.79-2.02 (m, 8H); 2.28-2.34 (m, 2H); 2.58-2.61 (m, 2H); 4.21 (q, 2H, J=7.1 Hz); 4.68 (s, 2H); 6.85 (s, 1H); 7.09 (d, 1H, J=7.8 Hz); 7.15-7.19 (m, 3H); 7.44 (d, 2H, J=8.1 Hz).

### Example 14.48.

### 2-butyl-1-[(3′-((1-(1-benzyloxymethyltetrazol-5-yl)-1-ethylmethyl)oxy)-biphenyl-4-yl)methyl]-4-spirocyclopentyl-1H-imidazol-5(4H)-one

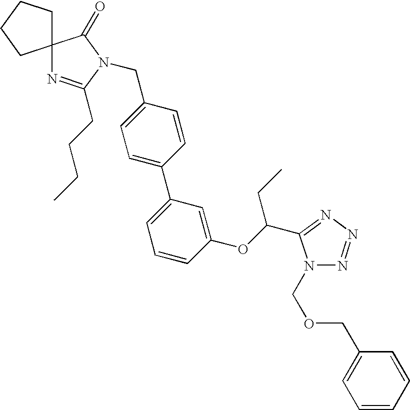

Prepared following the general procedure previously described (Method 14A) using 2-butyl-1-[(3′-hydroxybiphenyl-4-yl)methyl]-4-spirocyclopentyl-1H-imidazol-5(4H)-one (example 13.27) and 1-(1-(benzyloxymethyl)-1H-tetrazol-5-yl)propyl methanesulfonate (example 11.3). The product was chromatographed over silica gel (eluent petroleum ether/ethyl acetate 70/30). The product was obtained as a colorless oil.

Yield: 81%

Rf (petroleum ether/ethyl acetate 60/40): 0.38

IR: νCO 1719 cm⁻¹

NMR ¹H (CDCl₃): 0.89 (t, 3H, J=7.5 Hz); 1.10 (t, 3H, J=7.5 Hz); 1.24-1.43 (m, 2H); 1.54-1.66 (m, 2H); 1.80-2.02 (m, 8H); 2.15-2.38 (m, 4H); 4.60 (s, 2H); 4.73 (s, 2H); 5.60 (t, 1H, J=5 Hz); 5.91 (s, 2H); 6.99 (d, 1H, J=7.5 Hz); 7.14-7.34 (m, 10H); 7.51-7.54 (m, 2H).

MS (ESI): 607 (M+H)

### Example 15.

### Compounds of General Formula (I) According to the Invention

Compounds of general formula (I) according to the invention can be prepared following various methods:

Method 15A: Saponification of ethyl or methyl esters

To a solution of ethyl or methyl ester (1eq) in ethanol was added a sodium hydroxide 1N solution (2 to 5eq). The reaction mixture was stirred for 12 hours at room temperature. Ethanol was evaporated under reduced pressure. The reaction mixture was acidified then extracted with dichloromethane. The organic layer was dried over sodium sulfate, filtered and concentrated under reduced pressure. The residue was chromatographed over silica gel or recrystallized.

Method 15B: Acid hydrolysis of tert-butyl esters.

To a solution of tert-butyl ester (1eq) in dichloromethane was added trifluoroacetic acid (69eq). The reaction mixture was stirred for 12 hours at room temperature. The reaction mixture was taken up in dichloromethane, washed with water, then with brine. The organic layer was dried over sodium sulfate, filtered, and concentrated under reduced pressure. The residue was chromatographed over silica gel or crystallized.

Method 15C: Imidazolone substitution

Compounds can be prepared directly by imidazolone substitution with the appropriate brominated derivative in acetonitrile or N,N-dimethylformamide with potassium carbonate following one of the previously described methods (Method 10A and Method 10B).

Method 15D: Carbonyl reduction.

To a solution of he carbonyl derivative in trifluoroacetic acid was added triethylsilane (1eq) drop by drop under nitrogen atmosphere. The reaction mixture was stirred at 55° C. for 8 hours. The mixture was evaporated under reduced pressure and the residue was chromatographed over silica gel.

Method 15E: Tetrazole synthesis.

Nitrile (1eq), trimethylsilylazide (2eq) and bis(tributyltin) oxide (1eq) were dissolved in toluene in a schlenk tube. The reaction mixture was stirred at 110° C. for 48 hours under nitrogen atmosphere. The mixture was then stirred at room temperature for 12 hours, then acidified with a hydrochloric acid 6N solution to reach pH 1. The precipitate was taken up in dichloromethane. The organic layer was washed with water, dried over sodium sulfate, filtered and concentrated under reduced pressure. The residue was chromatographed over silica gel.

Method 15F: Tetrazole deprotection.

To a solution of the protected tetrazole (1eq) in dioxane was added hydrochloric acid 6N (45eq). The reaction mixture was stirred at 55° C. The organic layer was extracted three times with a sodium hydroxide 1N solution. The organic layer was then acidified to reach pH 2. The aqueous layer was then extracted with ethyl acetate. The organic layer was dried over magnesium sulfate and evaporated under reduced pressure. The residue was chromatographed over silica gel.

### Compound 1. 2-butyl-1-[(3′-((1-carboxy-1,1-dimethylmethyl)oxy)biphenyl-4-yl)methyl]-4-spirocyclopentyl-1H-imidazol-5(4H)-one

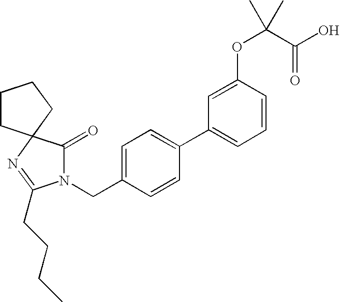

Prepared following the general procedure previously described (Method 15A) using 2-butyl-1-[(3′-((1-ethoxycarbonyl-1,1-dimethylmethyl)oxy)biphenyl-4-yl)methyl]-4-spirocyclopentyl-1H-imidazol-5(4H)-one (example 12.1). The product was chromatographed over silica gel (eluent dichloromethane/methanol 9/1) and preparative HPLC (gradient water/methanol/trifluoroacetic acid). The product was obtained as a white powder.

Yield: 8%

Rf (dichloromethane/methanol 95/5): 0.30

MP: 169-171° C. IR: νCO: 1727 cm⁻¹

NMR ¹H (DMSO-d₆): 0.79 (t, 3H, J=7.3 Hz); 1.25 (sext, 2H, J=7.6 Hz); 1.51 (quint, 2H, J=7.3 Hz); 1.54 (s, 6H); 1.65-1.95 (m, 8H); 2.34 (t, 2H, J=7.3 Hz); 4.72 (s, 2H); 6.81 (dd, 1H, J=1.8 Hz, J=7.3 Hz); 7.06 (t, 1H, J=2 Hz); 7.22-7.26 (m, 3H); 7.35 (t, 1H, J=7.9 Hz); 7.6 (d, 2H, J=8.2 Hz); 13.1 (s, 1H),

MS (MALDI-TOF): 463 (M+H)

### Compound 2. 2-butyl-1-[(4′-((1-carboxy-1,1-dimethylmethyl)oxy)biphenyl-4- yl)methyl]-4-spirocyclopentyl-1H-imidazol-5(4H)-one

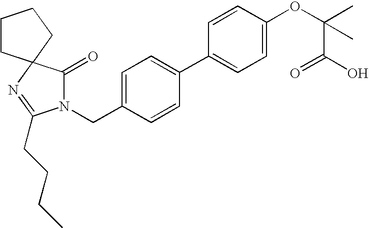

Prepared following the general procedure previously described (Method 15A) using 2-butyl-1-[(4′-((1-ethoxycarbonyl-1,1-dimethylmethyl)oxy)biphenyl-4-yl)methyl]-4-spirocyclopentyl-1H-imidazol-5(4H)-one (example 12.2). The product was obtained as a white powder which was recrystallized in acetonitrile.

Yield: 30%

Rf (dichloromethane/methanol 9/1): 0.15

MP: 180° C. IR: νCO: 1735 cm⁻¹

NMR ¹H (DMSO-d₆): 0.79 (t, 3H, J=7.4 Hz); 1.20-1.35 (m, 2H); 1.45-1.60 (m, 2H); 1.54 (s, 6H); 1.65-1.75 (m, 2H); 1.75-1.95 (m, 6H); 2.38 (m, 2H); 4.73 (s, 2H); 6.88 (d, 2H, J=8.1 Hz); 7.22 (d, 2H, J=8.1 Hz); 7.57 (d, 2H, J=8.1 Hz); 7.61 (d, 2H, J=8.1 Hz), 13.11 (s, 1H).

MS (APCI): 463 (M+H)

### Compound 3. 2-butyl-1-[2-(4-((1-carboxy-1,1-dimethylmethyl)oxy)phenyl)ethyl]-4-spirocyclopentyl-1H-imidazol-5(4H)-one

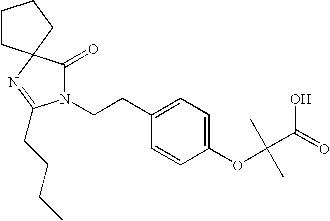

Prepared following the general procedure previously described (Method 15A) using 2-butyl-1-[2-(4-((1-ethoxycarbonyl-1,1-dimethylmethyl)oxy)phenyl)ethyl]-4-spirocyclopentyl-1H-imidazol-5(4H)-one (example 12.3). The product was chromatographed over silica gel (eluent dichloromethane/methanol 95/5). The product was obtained as a white powder.

Yield: 12%

Rf (dichloromethane/methanol 9/1): 0.40

MP: 50-53° C. IR: νCO: 1773 cm⁻¹; 1730 cm⁻¹

NMR ¹H (CDCl₃): 0.87 (t, 3H, J=7.3 Hz); 1.25 (sext, 2H, J=7.3 Hz); 1.47 (quint, 2H, J=7.3 Hz); 1.66 (s, 6H); 1.88 (t, 2H, J=7.9 Hz); 1.95-2.20 (m, 8H); 2.92 (t, 2H, J=5.8 Hz); 3.77 (t, 2H, J=5.8 Hz); 6.88 (m, 4H).

MS (ESI): 402 (M+H)

### Compound 4. 1-[(5′-bromo-2′-((1-carboxy-1,1-dimethylmethyl)oxy)biphenyl-4-yl)methyl]-2-butyl-4-spirocyclopentyl-1H-imidazol-5(4H)-one

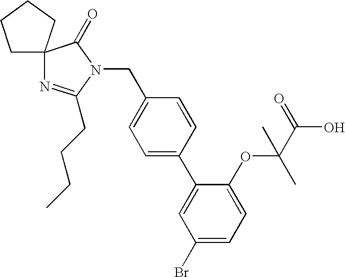

Prepared following the general procedure previously described (Method 15A) using 1-[(5′-bromo-2′-((1-ethoxycarbonyl-1,1-dimethylmethyl)oxy)biphenyl-4-yl)methyl]-2-butyl-4-spirocyclopentyl-1H-imidazol-5(4H)-one (example 12.4). The product was chromatographed over silica gel (eluent dichloromethane/methanol 9/1), then purified by preparative HPLC (elution gradient water/methanol/trifluoroacetic acid). The product was obtained as a white powder.

Yield: 18%

Rf (dichloromethane/methanol 9/1): 0.40

IR: νCO: 1773 cm⁻¹; 1730 cm⁻¹; 1626 cm⁻¹

NMR ¹H (CDCl₃): 0.90 (t, 3H, J=7.3 Hz); 1.30-1.43 (m, 2H); 1.39 (s, 6H); 1.60-1.72 (quint, 2H, J=7.9 Hz); 1.95-2.25 (m, 8H); 2.67 (t, 2H, J=7.9 Hz); 4.88 (s, 2H); 6.95 (d, 1H, J=8.8 Hz); 7.2 (d, 2H, J=7.9 Hz); 7.38 (dd, 1H, J=8.8 Hz, J=2.6 Hz); 7.45 (d, 1H, J=2.6 Hz); 7.50 (d, 2H, J=7.9 Hz).

MS (ESI): 541-542-543 (M+H)

### Compound 5. 2-butyl-1-[[4-[(3-((1-carboxyl-1,1-dimethylmethyl)oxy)phenyl)carbonyl]phenyl]methyl]-4-spirocyclopentyl-1H-imidazol-5(4H)-one

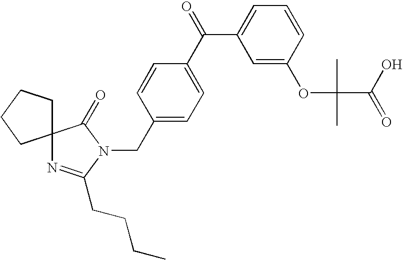

Prepared following the general procedure previously described (Method 15A) using 2-butyl-1-[[4-[(3-((1-ethoxycarbonyl-1,1-dimethylmethyl)oxy)phenyl)carbonyl]phenyl]methyl]-4-spirocyclopentyl-1H-imidazol-5(4H)-one (example 12.5). The product was chromatographed over silica gel (eluent dichloromethane/methanol 9/1). The product was obtained as a white powder.

Yield: 8%

Rf (dichloromethane/methanol 9/1): 0.40

IR: νCO: 1726 cm⁻¹

NMR ¹H (CDCl₃): 0.84 (t, 3H, J=7.3 Hz); 1.31 (sext, 2H, J=7.6 Hz); 1.50-1.68 (m, 8H); 1.75-2.10 (m, 8H); 2.36 (t, 2H, J=7.6 Hz); 4.78 (s, 2H); 7.12-7.40 (m, 6H); 7.75 (d, 2H, J=7.9 Hz).

MS (ESI): 491 (M+H)

### Compound 6. 2-butyl-1-[(2′-((1-carboxy-1,1-dimethylmethyl)oxy)biphenyl-4-yl)methyl]-4-spirocyclopentyl-1H-imidazol-5(4H)-one

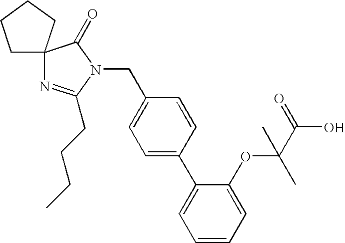

Prepared following the general procedure previously described (Method 15A) using 2-butyl-1-[(2′-((1-ethoxycarbonyl-1,1-dimethylmethyl)oxy)biphenyl-4-yl)methyl]-4-spirocyclopentyl-1H-imidazol-5(4H)-one (example 12.4). The product was chromatographed over silica gel (eluent dichloromethane/methanol 9/1), then purified by preparative HPLC (elution gradient water/methanol/trifluoroacetic acid).The product was obtained as a white powder.

Yield: 30%

Rf (dichloromethane/methanol 95/5): 0.30

MP: 74-80° C. IR: νCO: 1735 and 1627 cm⁻¹

NMR ¹H (CDCl₃): 0.86 (t, 3H, J=7.3 Hz); 1.32 (sext, 2H, J=7.6 Hz); 1.41 (s, 6H); 1.55 (quint, 2H, J=7.3 Hz); 1.85-2.10 (m, 8H); 2.37 (t, 2H, J=7.3 Hz); 4.74 (s, 2H); 7.03 (d, 1H, J=7.9 Hz); 7.11 (t, 1H, J=7.3 Hz); 7.17-7.24 (m, 3H); 7.32 (d, 1H, J=7.3 Hz); 7.5 (d, 2H, J=7.9 Hz).

MS (ESI): 463 (M+H)

### Compound 7. 2-butyl-1-[[4-[(2-((1-carboxyl-1,1-dimethylmethyl)oxy)phenyl)carbonyl]phenyl]methyl]-4-spirocyclopentyl-1H-imidazol-5(4H)-one

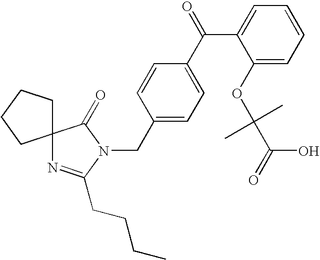

Prepared following the general procedure previously described (Method 15A) using 2-butyl-1-[[4-[(2-((1-ethoxycarbonyl-1,1-dimethylmethyl)oxy)phenyl)carbonyl]phenyl]methyl]-4-spirocyclopentyl-1H-imidazol-5(4H)-one (example 12.6). The product was chromatographed over silica gel (eluent dichloromethane/methanol 9/1). The product was obtained as a white powder.

Yield: 43%

Rf (dichloromethane/methanol 9/1): 0.25

MP: 85-90° C. IR: νCO: 1727, 1662, and 1633 cm⁻¹

NMR ¹H (DMSO-d₆): 0.78 (t, 3H, J=7.3 Hz); 1.12 (s, 6H); 1.23 (sext, 2H, 7.6 Hz); 1.44 (quint, 2H, J=7.9 Hz); 1.65-1.83 (m, 8H); 2.29 (t, 2H, J=7.3 Hz); 4.76 (s, 2H); 6.84 (d, 1H, J=8.5 Hz); 7.03 (t, 1H, J=7.3 Hz); 7.26 (d, 2H, J=7.9 Hz); 7.36 (d, 1H, J=8.5 Hz); 7.42 (t, 1H, J=7.3 Hz); 7.70 (d, 2H, J=8.2 Hz).

MS (ESI): 491 (M+H)

### Compound 8. 2-butyl-1-[2-(3-((1-carboxy-1,1-dimethylmethyl)oxy)phenyl)ethyl]-spirocyclopentyl-1H-imidazol-5(4H)-one

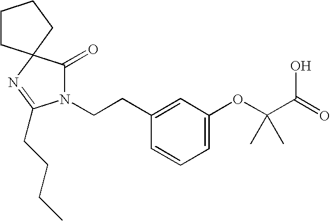

Prepared following the general procedure previously described (Method 15A) using 2-butyl-1-[2-(3-((1-ethoxycarbonyl-1,1-dimethylmethyl)oxy)phenyl)ethyl]-spirocyclopentyl-1H-imidazol-5(4H)-one (example 12.7). The product was chromatographed over silica gel (eluent dichloromethane/methanol 9/1). The product was obtained as a colorless oil.

Yield: 38%

Rf (dichloromethane/methanol 95/5): 0.30

IR: νCO (ester): 1732 cm⁻¹

NMR ¹H (CDCl₃): 0.88 (t, 3H, J=7.3 Hz); 1.20-1.40 (sext, 2H, J=7.3 Hz); 1.49-1.66 (quint, 2H, J=7.6 Hz); 1.66 (s, 6H); 1.90-2.15 (m, 6H); 2.16-2.30 (m, 4H); 2.96 (t, 2H, J=5.8 Hz); 3.95 (t, 2H, J=5.8 Hz); 6.47 (s, 1H); 6.87 (t, 2H, J=8.8 Hz); 7.26 (t, 1H, J=7.9 Hz)

MS (ESI): 401 (M+H)

### Compound 9. 2-butyl-1-[[4-[(4-((1-carboxyl-1,1-dimethylmethyl)oxy)phenyl)carbonyl]phenyl]methyl]-4-spirocyclopentyl-1H-imidazol-5(4H)-one

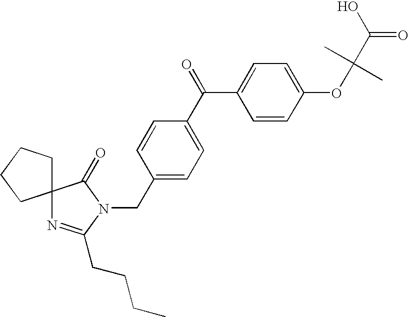

Prepared following the general procedure previously described (Method 15A) using 2-butyl-1-[[4-[(4-((1-ethoxycarbonyl-1,1-dimethylmethyl)oxy)phenyl)carbonyl]phenyl]methyl]-4-spirocyclopentyl-1H-imidazol-5(4H)-one (example 12.8). The product was chromatographed over silica gel (eluent dichloromethane/methanol 9/1). The product was obtained as a white powder.

Yield: 43%

Rf (dichloromethane/methanol 9/1): 0.25

MP: 90-99° C. IR: νCO: 1726 cm⁻¹

NMR ¹H (CDCl₃): 0.78 (t, 3H, J=7 Hz); 1.25 (sext, 2H, J=7.3 Hz); 1.46 (quint, 2H, J=7.3 Hz); 1.53 (s, 6H); 1.67-1.84 (m, 8H); 2.33 (t, 2H, J=7.6 Hz); 4.78 (s, 2H); 6.89 (d, 2H, J=8.8 Hz); 7.29 (d, 2H, J=8.2 Hz); 7.66 (m, 4H).

MS (ESI): 491 (M+H)

### Compound 10. 2-butyl-1-[2-(2-((1-carboxy-1,1-dimethylmethyl)oxy)phenyl)ethyl]-4-spirocyclopentyl-1H-imidazol-5(4H)-one

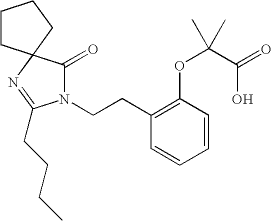

Prepared following the general procedure previously described (Method 15A) using 2-butyl-1-[2-(2-((1-ethoxycarbonyl-1,1-dimethylmethyl)oxy)phenyl)ethyl]-4-spirocyclopentyl-1H-imidazol-5(4H)-one (example 12.9). The product was chromatographed over silica gel (eluent dichloromethane/methanol 9/1). The product was obtained as a white powder.

Yield: 56%

Rf (dichloromethane/methanol 9/1): 0.40

MP: 51-57° C. IR: νCO (ester): 1732 cm⁻¹; νCO (lactone): 1623 cm⁻¹

NMR ¹H (CDCl₃): 0.84 (t, 3H, J=7.3 Hz); 1.31 (quint, 2H, J=7.6 Hz); 1.54 (m, 8H); 1.96 (m, 8H); 2.36 (t, 2H, J=7.6 Hz); 2.95 (t, 2H, J=5.9 Hz); 3.95 (t, 2H, J=5.9 Hz); 4.77 (s, 2H); 7.13-7.37 (m, 2H); 7.75 (d, 2H, J=7.9 Hz).

MS (ESI): 491 (M+H)

### Compound 11. 2-butyl-1-[[4-[(3-((1-carboxyl-1,1-dimethylmethyl)oxy)phenyl)carbonyl]phenyl]methyl]-4-spirocyclohexyl-1H-imidazol-5(4H)-one

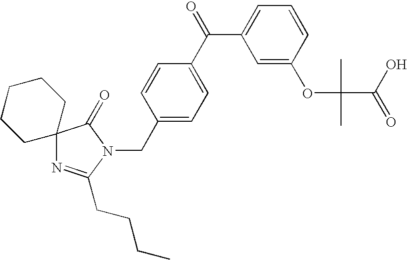

Prepared following the general procedure previously described (Method 15A) using 2-butyl-1-[[4-[(3-((1-ethoxycarbonyl-1,1-dimethylmethyl)oxy)phenyl)carbonyl]-phenyl]methyl]-4-spirocyclohexyl-1H-imidazol-5(4H)-one (example 12.10). The product was chromatographed over silica gel (eluent dichloromethane/methanol 9/1). The product was obtained as a white powder.

Yield: 56%

Rf (dichloromethane/methanol 9/1): 0.30

MP: 68-75° C. IR: νCO: 1726 cm⁻¹

NMR ¹H (CDCl₃): 0.84 (t, 3H, J=7.3 Hz); 1.33 (sext, 2H, J=7.6 Hz); 1.51-1.78 (m, 18H); 2.38 (t, 2H, J=7 Hz); 4.77 (s, 2H); 7.08-7.52 (m, 6H); 7.75 (d, 2H, J=7.9 Hz).

MS (ESI): 505 (M+H)

### Compound 12. 1-[(6′-bromo-3′-((1-carboxy-1,1-dimethylmethyl)oxy)biphenyl-4-yl)methyl]-2-butyl-4-spirocyclopentyl-1H-imidazol-5(4H)-one

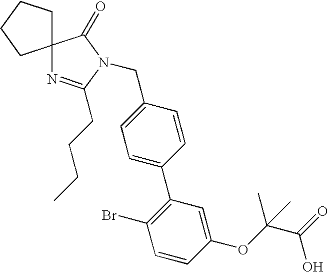

Prepared following the general procedure previously described (Method 15A) using 1-[(6′-bromo-3′-((1-ethoxycarbonyl-1,1-dimethylmethyl)oxy)biphenyl-4-yl)methyl]-2-butyl-4-spirocyclopentyl-1H-imidazol-5(4H)-one (example 12.1). The product was chromatographed over silica gel (eluent dichloromethane/methanol 9/1), and then purified by preparative HPLC (gradient water/methanol/trifluoroacetic acid). The product was obtained as a white powder.

Yield: 7%

Rf (dichloromethane/methanol 9/1): 0.50

MP: 174-176° C. IR: νCO: 1737 cm⁻¹

NMR ¹H (CDCl₃): 0.80 (t, 3H, J=7.3 Hz); 1.27 (sext, 2H, J=7.9 Hz); 1.54 (quint, 2H, J=7.3 Hz); 1.64 (s, 6H); 1.84-2.03 (m, 8H); 2.42 (t, 2H, J=7.3 Hz); 4.74 (s, 2H); 6.82 (dd, 1H, J=8.8 Hz, J=1.6 Hz); 6.88 (d, 1H, J=2.9 Hz); 7.17 (d, 2H, J=8.2 Hz); 7.36 (d, 2H, J=8.5 Hz); 7.52 (d, 1H, J=8.8 Hz).

MS (ESI): 541-543 (M+H)

### Compound 13. 2-butyl-1-[(3′-((1-carboxy-1,1-dimethylmethyl)oxy)biphenyl-4-yl)methyl]-4,4-dimethyl-1H-imidazol-5(4H)-one

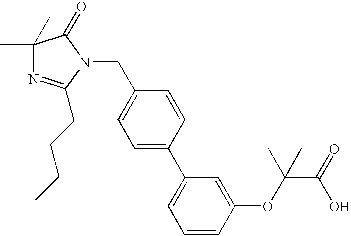

Prepared following the general procedure previously described (Method 15A) using 2-butyl-4,4-dimethyl-1-[(3′-((1-ethoxycarbonyl-1,1-dimethylmethyl)oxy)biphenyl-4-yl)methyl]-1H-imidazol-5(4H)-one (example 12.11). The product was chromatographed over silica gel (eluent dichloromethane/methanol 9/1). The product was then purified by preparative HPLC (gradient water/methanol/trifluoroacetic acid). The product was obtained as a white powder.

Yield: 27%

Rf (dichloromethane/methanol 95/5): 0.3

MP: 171-173° C. IR: νCO: 1730 cm⁻¹

NMR ¹H (DMSO-d₆): 0.79 (t, 3H, J=7.3 Hz); 1.23 (s, 6H); 1.30 (sext, 2H, J=7.3 Hz); 1.49 (quint, 2H, J=7.9 Hz); 1.54 (s, 6H); 2.34 (t, 2H, J=7.3 Hz); 4.71 (s, 2H); 6.81 (dd, 1H, J=8.2 Hz, J=1.8 Hz); 7.07 (s, 1H); 7.22-7.25 (m, 3H); 7.35 (t, 1H, J=7.9 Hz); 7.6 (d, 2H, J=8.2 Hz).

MS (ESI): 463 (M+H)

### Compound 14. 2-butyl-1-[[4-[(3-((1-carboxyl-1,1-dimethylmethyl)oxy)phenyl)carbonyl]phenyl]methyl]-4,4-dimethyl-1H-imidazol-5(4H)-one

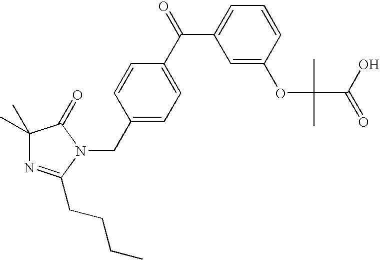

Prepared following the general procedure previously described (Method 15A) using 2-butyl-4,4-dimethyl-1-[[4-[(3-((1-ethoxycarbonyl-1,1-dimethylmethyl)oxy)phenyl)carbonyl]phenyl]methyl]-1H-imidazol-5(4H)-one (example 12.12). The product was purified by preparative HPLC (gradient water/methanol/trifluoroacetic acid). The product was obtained as a white powder.

Yield: 67%

Rf (dichloromethane/methanol 9/1): 0.35

MP: 167-169° C. IR: νCO: 1746 and 1661 cm⁻¹

NMR ¹H (CDCl₃): 0.84 (t, 3H, J=7.2 Hz); 1.33 (sext, 2H, J=7.3 Hz); 1.41 (s, 6H); 1.54-1.63 (m, 8H); 2.39 (t, 2H, J=7.3 Hz); 4.78 (s, 2H); 7.16 (d, 1H, J=7.6 Hz); 7.25-7.43 (m, 5H); 7.78 (d, 2H, J=8.2 Hz).

MS (ESI): 465 (M+H); 487 (M+Na); 503 (M+K)

### Compound 15. 2-butyl-1-[[4-[(3-((1-carboxy-1,1-dimethylmethyl)oxy)phenyl)carbonyl]phenyl]methyl]-4-phenyl-1H-imidazol-5(4H)-one

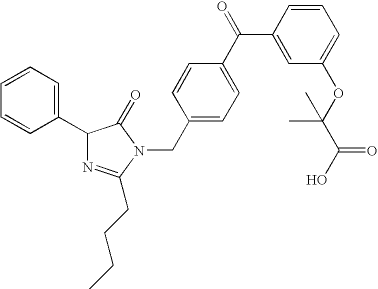

Prepared following the general procedure previously described (Method 15A) using 2-butyl-1-[[4-[(3-((1-ethoxycarbonyl-1,1-dimethylmethyl)oxy)phenyl)carbonyl]phenyl]methyl]-4-phenyl-1H-imidazol-5(4H)-one (example 12.13). The product was chromatographed over silica gel (eluent dichloromethane/methanol 95/5). The product was obtained as a brown solid.

Yield: 25%

Rf (dichloromethane/methanol 9/1): 0.35

MP: 115-120° C. IR: νCO: 1736 and 1656 cm⁻¹

NMR ¹H (CDCl₃): 0.84 (t, 3H, J=7.3 Hz); 1.15-1.29 (m, 2H); 1.46 (quint, 2H, J=7.3 Hz); 1.57 (s, 6H); 2.28 (t, 2H, J=7.6 Hz); 4.26 (s, 1H); 4.72 (s, 2H) 6.82 (m, 1H); 7.09 (m, 1H); 7.19-7.30 (m, 5H); 7.31 (m, 2H); 7.52 (d, 2H, J=8.2 Hz); 7.78 (d, 2H, J=8.2 Hz).

MS (ESI): 513 (M+H)

### Compound 16. 1-[(3′-((1-carboxy-1,1-dimethylmethyl)oxy)biphenyl-4-yl)methyl]-2-propyl-4-spirocyclopentyl-1H-imidazol-5(4H)-one

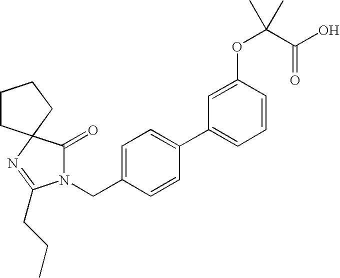

Prepared following the general procedure previously described (Method 15B) using 1-[(3′-((1-tert-butyloxycarbonyl-1,1-dimethylmethyl)oxy)biphenyl-4-yl)methyl]-2-propyl-4-spirocyclopentyl-1H-imidazol-5(4H)-one (example 12.14). The product was washed with ethyl acetate, filtered, and dried under high reduced pressure The product was obtained as a white powder.

Yield: 47%

Rf (dichloromethane/methanol 95/5): 0.35

MP: 134-190° C. IR: νCO: 1763 and 1731 cm⁻¹

NMR ¹H (CDCl₃): 0.88 (t, 3H, J=7.3 Hz); 1.59-1.72 (m, 8H); 1.90-2.12 (m, 8H); 2.52 (t, 2H, J=7.3 Hz); 4.75 (s, 2H); 6.94 (dd, 1H, J=7.3 Hz, J=1.8 Hz); 7.15 (s, 1H); 7.18 (d, 2H, J=7.6 Hz); 7.23 (d, 1H, J=7.3 Hz); 7.32 (t, 1H, J=7.9 Hz); 7.52 (d, 2H, J=8.2 Hz).

MS (ESI): 449 (M+H); 471 (M+Na); 487 (M+K)

### Compound 17. 1-[(3′-((1-carboxy-1,1-dimethylmethyl)oxy)biphenyl-4-yl)methyl]-2-ethyl-4-spirocyclopentyl-1H-imidazol-5(4H)-one

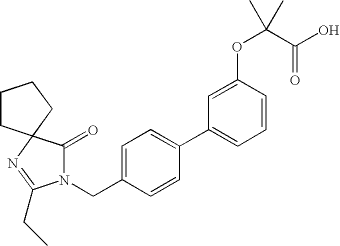

Prepared following the general procedure previously described (Method 15B) using 1-[(3′-((1-tert-butyloxycarbonyl-1,1-dimethylmethyl)oxy)biphenyl-4-yl)methyl]-2-ethyl-4-spirocyclopentyl-1H-imidazol-5(4H)-one (example 12.15). The product was obtained as a colorless oil.

Yield: 99%

Rf (dichloromethane/methanol 95/5): 0.35

IR: νCO: 1776 and 1730 cm⁻¹

NMR ¹H (CDCl₃): 1.20-1.32 (m, 3H); 1.65 (s, 6H); 1.92-2.31 (m; 8H); 2.80 (m, 2H); 4.89 (s, 2H); 6.92 (d, 1H, J=7.3 Hz); 7.16 (s, 1H); 7.19-7.27 (m, 3H); 7.35 (t, 1H, J=7.9 Hz); 7.58 (d, 2H, J=8.2 Hz).

MS (ESI): 433 (M−H)

### Compound 18. 1-[(3′-((1-carboxy-1,1-dimethylmethyl)oxy)biphenyl-4-yl)methyl]-2-methyl-4-spirocyclopentyl-1H-imidazol-5(4H)-one

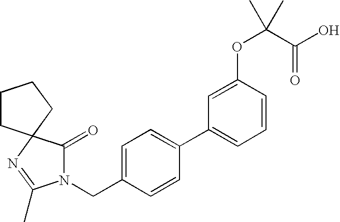

Prepared following the general procedure previously described (Method 15B) using 1-[(3′-((1-tert-butyloxycarbonyl-1,1-dimethylmethyl)oxy)biphenyl-4-yl)methyl]-2-methyl-4-spirocyclopentyl-1H-imidazol-5(4H)-one (example 12.16). The product was obtained as a white powder.

Yield: 99%

Rf (dichloromethane/methanol 95/5): 0.30

MP: 81-88° C. IR: νCO: 1776 and 1729 cm⁻¹

NMR ¹H (CDCl₃): 1.70 (s, 6H); 1.92-2.26 (m, 8H); 2.52 (s, 3H); 4.86 (s, 2H); 6.98 (d, 1H, J=7.3 Hz); 7.19 (s, 1H); 7.20-7.26 (m, 3H); 7.39 (t, 1H, J=7.9 Hz); 7.59 (d, 2H, J=8.2 Hz).

MS (ESI): 419 (M−H)

### Compound 19. 2-butyl-1-[(3′-((1-carboxy-1,1-dimethylmethyl)oxy)biphenyl-4-yl)methyl]-4-phenyl-1H-imidazol-5(4H)-one

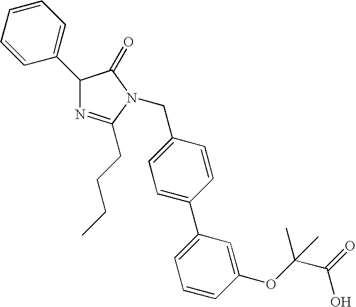

Prepared following the general procedure previously described (Method 15A) using 2-butyl-1-[(3′-((1-ethoxycarbonyl-1,1-dimethylmethyl)oxy)biphenyl-4-yl)methyl]-4-phenyl-1H-imidazol-5(4H)-one (example 12.17). The product was purified by preparative HPLC (gradient water/methanol/trifluoroacetic acid). The product was obtained as a white powder.

Yield: 30%

Rf (dichloromethane/methanol 95/5): 0.30

MP: 197-199° C. IR: νCO: 1692cm⁻¹

NMR ¹H (DMSO-d₆): 0.73 (t, 3H, J=7.3 Hz); 1.02-1.12 (sext, 2H, J=7.3 Hz); 1.31-1.42 (quint, 2H, J=7.3 Hz); 1.54 (s, 6H); 2.15-2.28 (m, 2H); 3.17-3.33 (m, 2H); 6.79 (dd, 1H, J=7.6 Hz, J=1.8 Hz); 7.04 (t, 1H, J=1.8 Hz); 7.17-7.25 (m, 3H); 7.27-7.41 (m, 4H); 7.42-7.50 (d, 2H, J=8.2 Hz); 7.60-7.67 (d, 2H, J=7.3 Hz); 10.62 (s, 1H); 13.12 (s, 1H).

MS (ESI): 463 (M+H)

### Compound 20. 2-butyl-1-[[4-[(4-((1-carboxy-1,1-dimethylmethyl)oxy)phenyl)carbonyl]phenyl]methyl]-4-phenyl-1H-imidazol-5(4H)-one

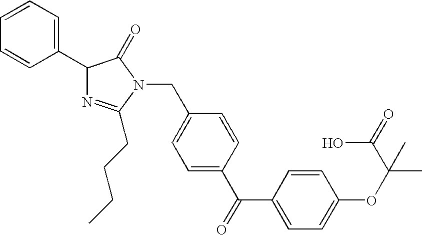

Prepared following the general procedure previously described (Method 15A) using 2-butyl-1-[[4-[(4-((1-ethoxycarbonyl-1,1-dimethylmethyl)oxy)phenyl)carbonyl]phenyl]methyl]-4-phenyl-1H-imidazol-5(4H)-one (example 12.18). The product was purified by preparative HPLC (gradient water/methanol/trifluoroacetic acid) The product was obtained as a white powder.

Yield: 46%

Rf (dichloromethane/methanol 9/1): 0.40

MP: 215-217° C.

IR: νCO: 1725 and 1650 cm⁻¹

NMR ¹H (DMSO-d₆): 0.76 (t, 3H, J=7.3 Hz); 1.02-1.15 (m; 2H); 1.30-1.41 (quint, 2H, J=7.3 Hz); 1.60 (s, 6H); 2.18-2.30 (m, 2H); 3.21-3.48 (m, 2H); 6.89 (d, 2H, J=8.8 Hz); 7.26-7.42 (m, 5H); 7.54-7.59 (d, 2H, J=7.9 Hz); 7.60-7.69 (m, 4H); 10.07 (s, 1H); 13.29 (s, 1H).

MS (ESI): 513 (M+H)

### Compound 21. 2-butyl-1-[(3′-((1-carboxy-1,1-dimethylmethyl)oxy)biphenyl-4-yl)methyl]-4-spirocyclohexyl-1H-imidazol-5(4H)-one

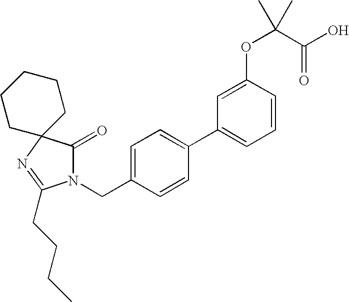

Prepared following the general procedure previously described (Method 15A) using 2-butyl-1-[(3′-((1-ethoxycarbonyl-1,1-dimethylmethyl)oxy)biphenyl-4-yl)methyl]-4-spirocyclohexyl-1H-imidazol-5(4H)-one (example 12.19). The product was purified by preparative HPLC (gradient water/methanol/trifluoroacetic acid). The product was obtained as a white powder.

Yield: 46%

Rf (dichloromethane/methanol 95/5): 0.30

MP: 160-162° C. IR: νCO: 1725 and 1629 cm⁻¹

NMR ¹H (DMSO-d₆): 0.79 (t, 3H, J=7.3 Hz); 1.25 (sext, 2H, J=7.6 Hz); 1.30-1.42 (m, 2H); 1.52 (quint, 2H, J=7.3 Hz); 1.54 (s, 6H); 1.59-1.78 (m, 8H); 2.35 (t, 2H, J=7.3 Hz); 4.72 (s, 2H); 6.81 (dd, 1H, J=7.9 Hz, J=1.5 Hz); 7.06 (t, 1H, J=2 Hz); 7.19-7.28 (m, 3H); 7.35 (t, 1H, J=7.9 Hz); 7.59 (d, 2H, J=8.2 Hz); 13.11 (s, 1H).

MS (ESI): 477 (M+H)

### Compound 22. 1-[(6′-bromo-3′-((1-carboxy-1,1-dimethylmethyl)oxy)biphenyl-4-yl)methyl]-2-butyl-4-spirocyclohexyl-1H-imidazol-5(4H)-one

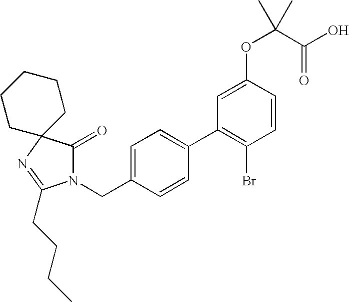

Prepared following the general procedure previously described (Method 15A) using 1-[(6′-bromo-3′-((1-ethoxycarbonyl-1,1-dimethylmethyl)oxy)biphenyl-4-yl)methyl]-2-butyl-4-spirocyclohexyl-1H-imidazol-5(4H)-one (example 12.19). The product was purified by preparative HPLC (gradient water/methanol/trifluoroacetic acid)The product was obtained as a white powder.

Yield: 5%

Rf (dichloromethane/methanol 9/1): 0.40

MP: 170-172° C. IR: νCO: 1736 and 1629 cm⁻¹

NMR ¹H (DMSO-d₆): 0.79 (t, 3H, J=7.3 Hz); 1.25 (sext, 2H, J=7.6 Hz); 1.32-1.41 (m, 2H); 1.49 (quint, 2H, J=7.3 Hz); 1.52 (s, 6H); 1.59-1.78 (m, 8H); 2.37 (t, 2H, J=7.3 Hz); 4.74 (s, 2H); 6.75-6.81 (m, 2H); 7.21 (d, 2H, J=8.2 Hz); 7.37 (d, 1H, J=8.2 Hz); 7.60 (d, 2H, J=9 Hz); 13.19 (s, 1H).

MS (ESI): 555-557 (M+H); 577-579 (M+Na)

### Compound 23. 2-butyl-1-[(3′-(cyanomethoxy)biphenyl-4-yl)methyl]-4-spirocyclopentyl-1H-imidazol-5(4H)-one

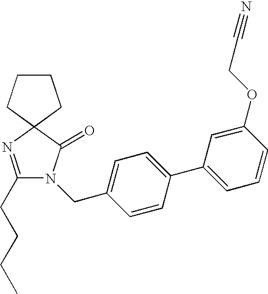

Prepared following the general procedure previously described (Method 15C) using 2-butyl-4-spirocyclopentyl-1H-imidazol-5(4H)-one (example 3.4) and 2-((4′-bromomethylbiphenyl-3-yl)oxy)acetonitrile (example 6.5). The product was chromatographed over silica gel (eluent dichloromethane/methanol 98/2). The product was obtained as a white powder.

Yield: 19%

Rf (dichloromethane/methanol 98/2): 0.34

MP: 128-130° C. IR: νCO: 1714 and 1638 cm⁻¹

NMR ¹H (CDCl₃): 0.87 (t, 3H, J=7.3 Hz); 1.30-1.40 (sext, 2H, J=7.6 Hz); 1.53-1.65 (quint, 2H, J=8.5 Hz); 1.80-2.10 (m, 8H); 2.35 (t, 2H, J=7.3 Hz); 4.73 (s, 2H); 4.83 (s, 2H); 6.94-7.01 (dd, 1H, J=8.2 Hz, J=2.6 Hz); 7.17 (s, 1H); 7.24 (d, 1H, J=8.2 Hz); 7.29 (d, 2H, J=7.6 Hz); 7.38-7.46 (t, 1H, J=7.9 Hz); 7.54 (d, 2H, J=8.2 Hz).

MS (ESI): 416 (M+H)

### Compound 24. 1-[(5′-bromo-2′-((1-carboxy-1,1-dimethylmethyl)oxy)biphenyl-4-yl)methyl]-2-butyl-4-spirocyclohexyl-1H-imidazol-5(4H)-one

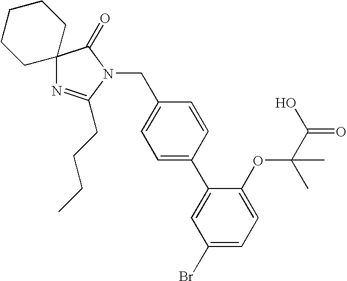

Prepared following the general procedure previously described (Method 15A) using 1-[(5′-bromo-2′-((1-ethoxycarbonyl-1,1-dimethylmethyl)oxy)biphenyl-4-yl)methyl]-2-butyl-4-spirocyclohexyl-1H-imidazol-5(4H)-one (example 12.20). The product was purified by preparative HPLC (elution gradient water/methanol/trifluoroacetic acid). The product was obtained as a yellowish oil.

Yield: 14%

Rf (dichloromethane/methanol 9/1): 0.30

IR: νCO: 1777 and 1627 cm⁻¹

NMR ¹H (DMSO-d₆): 0.80 (t, 3H, J=7.3 Hz); 1.32 (sext, 2H, J=7.9 Hz); 1.41 (s, 6H); 1.51 (quint, 2H, J=7.3 Hz); 1.55-1.72 (m, 8H); 2.66 (m, 4H); 4.87 (s, 2H); 6.79 (d, 1H, J=8.5 Hz); 7.28 (d, 2H, J=8.2 Hz); 7.43-7.48 (m, 2H); 7.53 (d, 2H, J=8.2 Hz).

MS (APCI): 557-558 (M+H)

### Compound 25. 2-butyl-1-[(4′-((1-carboxy-1,1-dimethylmethyl)oxy)biphenyl-4-yl)methyl]-4-spirocyclohexyl-1H-imidazol-5(4H)-one

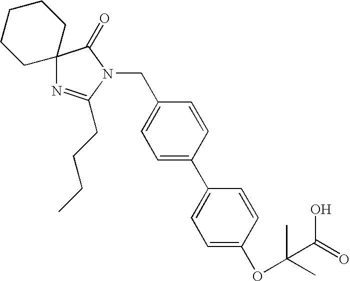

Prepared following the general procedure previously described (Method 15A) using 2-butyl-1-[(4′-((1-ethoxycarbonyl-1,1-dimethylmethyl)oxy)biphenyl-4-yl)methyl]-4-spirocyclohexyl-1H-imidazol-5(4H)-one (example 12.21). The product was purified by preparative HPLC (elution gradient water/methanol/trifluoroacetic acid). The product was obtained as a white powder.

Yield: 64%

Rf (dichloromethane/methanol 9/1): 0.25

MP: 75-77° C. IR: νCO: 1729 and 1627 cm⁻¹

NMR ¹H (DMSO-d₆): 0.79 (t, 3H, J=7.3 Hz); 1.21-1.35 (sext, 2H, J=7.3 Hz); 1.42-1.55 (m, 2H); 1.54 (s, 6H); 1.59-1.76 (m, 8H); 2.50 (m, 4H); 4.77 (s, 2H); 6.88 (d, 2H, J=8.8 Hz); 7.23 (d, 2H, J=7.9 Hz); 7.56 (d, 2H, J=8.8 Hz); 7.61 (d, 2H, J=8.5 Hz).

MS (APCI): 477 (M+H)

### Compound 26. 1-[(5′-bromo-2′-((1-carboxy-1,1-dimethylmethyl)oxy)biphenyl-4-yl)methyl]-2-butyl-4-phenyl-1H-imidazol-5(4H)-one

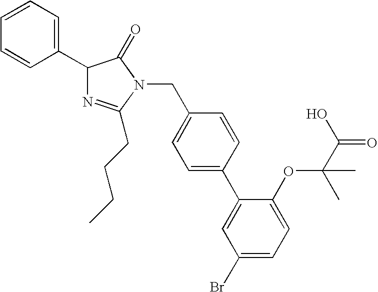

Prepared following the general procedure previously described (Method 15A) using 1-[(5′-bromo-2′-((1-ethoxycarbonyl-1,1-dimethylmethyl)oxy)biphenyl-4-yl)methyl]-2-butyl-4-phenyl-1H-imidazol-5(4H)-one (example 12.22). The product was purified by preparative HPLC (elution gradient water/methanol/trifluoroacetic acid)The product was obtained as a white powder.

Yield: 7%

Rf (dichloromethane/methanol 95/5): 0.30

MP: 199-201° C. IR: νCO: 1786 and 1666 cm⁻¹

NMR ¹H (DMSO-d₆): 0.78 (t, 3H, J=7.3 Hz); 1.02-1.18 (sext, 2H, J=7.9 Hz); 1.38 (m, 2H); 1.40 (s, 6H); 2.40 (t, 2H, J=7.6 Hz); 3.20-3.50 (m, 3H); 6.80 (d, 1H, J=8.8 Hz); 7.18 (d, 2H, J=8.2 Hz); 7.30-7.50 (m, 7H); 7.62 (d, 2H, J=8.2 Hz).

MS (ESI): 579-580-581 (M+H)

### Compound 27. 1-[(3′-((1-carboxy-1,1-dimethylmethyl)oxy)biphenyl-4-yl)methyl]-2-(2-methyl)propyl-4-spirocyclopentyl-1H-imidazol-5(4H)-one

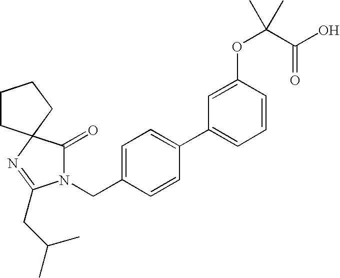

Prepared following the general procedure previously described (Method 15B) using 1-[(3′-((1-tert-butyloxycarbonyl-1,1-dimethylmethyl)oxy)biphenyl-4-yl)methyl]-2-(2-methyl)propyl-4-spirocyclopentyl-1H-imidazol-5(4H)-one (example 12.23). The product was crystallized in a dichloromethane/diethyl ether mixture. The product was obtained as a white powder.

Yield: 61%

Rf (dichloromethane/methanol 95/5): 0.32

MP: 213-216° C. IR: νCO: 1731 and 1630 cm⁻¹

NMR ¹H (CDCl₃): 0.95 (d, 6H, J=6.7 Hz); 1.69 (s, 6H); 1.90-2.20 (m, 10H); 2.50 (m, 1H); 4.80 (s, 2H); 6.95 (d, 1H, J=7.3 Hz); 7.19 (s, 1H); 7.20 (d, 2H, J=8.2 Hz); 7.27 (m, 1H); 7.36 (t, 1H, J=7.9 Hz); 7.55 (d, 2H, J=8.2 Hz).

MS (ESI): 461 (M−H)

### Compound 28. 2-benzyl-1-[(3′-((1-carboxy-1,1-dimethylmethyl)oxy)biphenyl-4-yl)methyl]-4-spirocyclopentyl-1H-imidazol-5(4H)-one

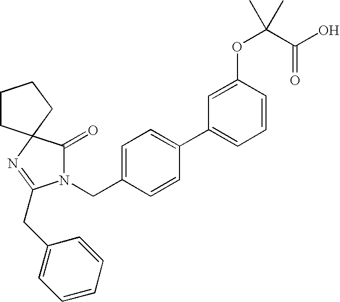

Prepared following the general procedure previously described (Method 15B) using 2-benzyl-1-[(3′-((1-tert-butyloxycarbonyl-1,1-dimethylmethyl)oxy)biphenyl-4-yl)methyl]-4-spirocyclopentyl-1H-imidazol-5(4H)-one (example 12.24). The product was chromatographed over silica gel (eluent cyclohexane/ethyl acetate 9/1 to 0/1). The product was obtained as a white powder.

Yield: 38%

Rf (dichloromethane/methanol 95/5): 0.30

MP: 149-153° C. IR: νCO: 1736 and 1627 cm⁻¹

NMR ¹H (CDCl₃): 1.68 (s, 6H); 1.90-2.15 (m, 8H); 3.75 (s, 2H); 4.49 (s, 2H); 6.92 (dd, 1H, J=7.3 Hz, J=1.8 Hz); 7.08 (d, 2H, J=7.6 Hz); 7.11-7.22 (m, 4H); 7.24-7.38 (m, 4H); 7.45 (d, 2H, J=8.2 Hz).

MS (ESI): 495 (M−H)

### Compound 29. 1-[(3′-((1-carboxy-1,1-dimethylmethyl)oxy)biphenyl-4-yl)methyl]-2-cyclopropyl-4-spirocyclopentyl-1H-imidazol-5(4H)-one

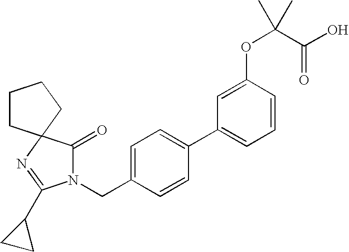

Prepared following the general procedure previously described (Method 15B) using 1-[(3′-((1-tert-butyloxycarbonyl-1,1-dimethylmethyl)oxy)biphenyl-4-yl)methyl]-2-cyclopropyl-4-spirocyclopentyl-1H-imidazol-5(4H)-one (example 12.25). The product was chromatographed over silica gel (eluent cyclohexane/ethyl acetate 9/1 to 0/1). The product was obtained as a white powder.

Yield: 12%

Rf (dichloromethane/methanol 95/5): 0.30

MP: 175-177° C.

IR: νCO: 1734 and 1600 cm⁻¹

NMR ¹H (DMSO-d₆): 0.80 (d, 4H, J=7.1 Hz); 1.53 (s, 6H); 1.61 (m, 1H); 1.72-1.90 (m, 8H); 4.84 (s, 2H); 6.82 (dd, 1H, J=7.3 Hz, J=1.8 Hz); 7.09 (s, 1H); 7.22-7.31 (m, 3H); 7.35 (t, 1H, J=7.9 Hz); 7.6 (d, 2H, J=8.2 Hz).

MS (ESI): 445 (M−H)

### Compound 30. 1-[(3′-((1-carboxy-1,1-dimethylmethyl)oxy)biphenyl-4-yl)methyl]-2-(thiophen-2-yl)methyl-4-spirocyclopentyl-1H-imidazol-5(4H)-one

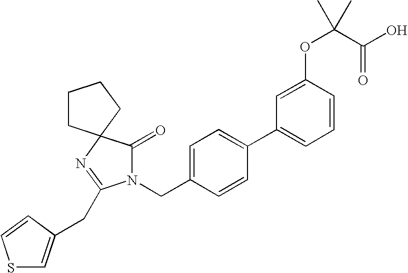

Prepared following the general procedure previously described (Method 15B) using 1-[(3′-((1-tert-butyloxycarbonyl-1,1-dimethylmethyl)oxy)biphenyl-4-yl)methyl]-2-(thiophen-2-yl)methyl-4-spirocyclopentyl-1H-imidazol-5(4H)-one (example 12.26). The product was chromatographed over silica gel (eluent cyclohexane/ethyl acetate 9/1 to 0/1). The product was obtained as a white powder.

Yield: 15%

Rf (dichloromethane/methanol 95/5): 0.31

MP: 102-110° C.

IR: νCO: 1738 and 1625 cm⁻¹

NMR ¹H (CDCl₃): 1.70 (s, 6H); 1.90-2.19 (m, 8H); 3.75 (s, 2H); 4.59 (s, 2H); 6.91-7.02 (m, 2H); 7.07 (s, 1H); 7.09-7.20 (m, 3H); 7.21-7.40 (m, 3H); 7.49 (d, 2H, J=8.2 Hz).

MS (ESI): 501 (M−H)

### Compound 31. 2-butyl-1-[[4-[(4-((1-carboxy-1,1-dimethylmethyloxy)phenyl)methyl]phenyl]methyl]-4-spirocyclopentyl-1H-imidazol-5(4H)-one

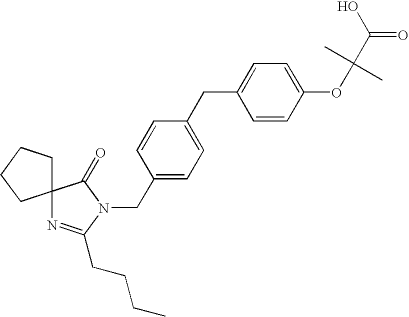

Prepared following the general procedure previously described (Method 15A) using 2-butyl-1-[[4-[(4-((1-ethoxycarbonyl-1,1-dimethylmethyloxy)phenyl)methyl]phenyl]methyl]-4-spirocyclopentyl-1H-imidazol-5(4H)-one (example 12.27).

Yield: 29%

This product can also be prepared following the general procedure previously described (Method 15D) using 2-butyl-1-[[4-[(4-((1-carboxy-1,1-dimethylmethyl)oxy)phenyl)carbonyl]phenyl]methyl]-4-spirocyclopentyl-1H-imidazol-5(4H)-one (EXAMPLE 15-Compound 9). The product was chromatographed over silica gel (eluent dichloromethane/methanol 95/5). The product was obtained as a white powder.

Yield: 20%

Rf (dichloromethane/ethyl acetate 95/5): 0.50

MP: 147-149° C. IR: νCO: 1733 cm⁻¹

NMR ¹H (CDCl₃): 0.80 (t, 3H, J=7.3 Hz); 1.27 (sext, 2H, J=7.6 Hz); 1.51 (quint, 2H, J=7.6 Hz); 1.58 (s, 6H); 1.84-2.01 (m, 8H); 2.36 (t, 2H, J=7.6 Hz); 3.89 (s, 2H); 4.66 (s, 2H); 6.84 (d, 2H, J=8.5 Hz); 7.00-7.06 (m, 4H); 7.13 (d, 2H, J=8.2 Hz).

MS (ESI): 475 (M−H)

### Compound 32. 2-butyl-1-[(4′-((4-carboxy-4,4-dimethylbutan-1-yl)oxy)biphenyl-4-yl)methyl]-4-spirocyclopentyl-1H-imidazol-5(4H)-one

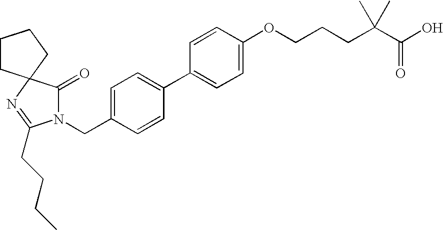

Prepared following the general procedure previously described (Method 15A) using 2-butyl-1-[(4′-((4-methyloxycarbonyl-4,4-dimethylbutan-1-yl)oxy)biphenyl-4-yl)methyl]-4-spirocyclopentyl-1H-imidazol-5(4H)-one (example 12.28). The product was chromatographed over silica gel (eluent dichloromethane/methanol 95/5, then cyclohexane/ethyl acetate 7/3). The product was obtained as a yellow powder.

Yield: 28%

Rf (dichloromethane/methanol 95/5): 0.30

IR: νCO: 1729 cm⁻¹

NMR ¹H (CDCl₃): 0.88 (t, 3H, J=7.2 Hz); 1.25 (s, 6H); 1.20-1.40 (m, 2H); 1.49 (quint, 2H, J=8.4 Hz); 1.68-1.90 (m, 6H); 1.91-2.10 (m, 4H); 2.18 (t, 2H, 8 Hz); 2.36 (t, 2H, J=7.6 Hz); 3.98 (t, 2H, J=6 Hz); 4.66 (s, 2H); 6.95 (d, 2H, J=9.2 Hz); 7.21 (d, 1H, J=8 Hz); 7.30 (d, 1H, J=8.8 Hz); 7.42-7.55 (m, 4H).

MS (ESI): 503 (M−H)

### Compound 33. 2-butyl-1-[(3′-((4-carboxy-4,4-dimethylbutan-1-yl)oxy)biphenyl-4-yl)methyl]-4-spirocyclopentyl-1H-imidazol-5(4H)-one

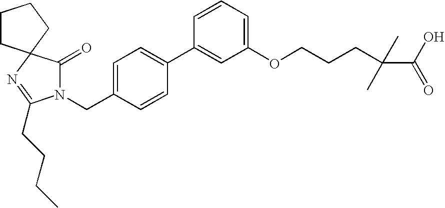

Prepared following the general procedure previously described (Method 15A) using 2-butyl-1-[(3′-((4-methoxycarbonyl-4,4-dimethylbutan-1-yl)oxy)biphenyl-4-yl)methyl]-4-spirocyclopentyl-1H-imidazol-5(4H)-one (example 12.29). The product was chromatographed over silica gel (eluent cyclohexane/ethyl acetate 6/4, then 7/3). The product was obtained as a white oil.

Yield: 20%

Rf (dichloromethane/methanol 95/5): 0.30

IR: νCO: 1736 cm⁻¹

NMR ¹H (CDCl₃): 0.85 (t, 3H, J=8 Hz); 1.15 (s, 6H); 1.26 (sext, 2H, J=7.6 Hz); 1.55-1.75 (m, 6H); 1.88 (m, 2H); 1.92-2.10 (m, 6H); 2.40 (t, 2H, J=8 Hz); 3.95 (t, 2H, J=6 Hz); 4.66 (s, 2H); 6.92 (d, 1H, J=8 Hz); 7.05 (t, 1H, J=2 Hz); 7.11 (d, 2H, J=8 Hz); 7.28 (d, 2H, J=8 Hz); 7.51 (d, 2H, J=8 Hz).

MS (ESI): 503 (M−H)

### Compound 34. 2-butyl-1-[[4-[(4-((1-carboxy-1,1-dimethylmethyloxy)phenyl)oxy]phenyl]methyl]-4-spirocyclopentyl-1H-imidazol-5(4H)-one

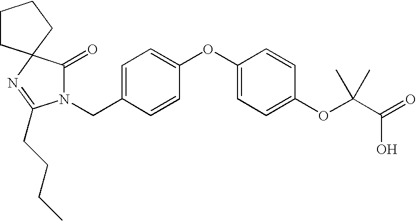

Prepared following the general procedure previously described (Method 15B) using 2-butyl-1-[[4-[(4-((1-tert-butyloxycarbonyl-1,1-dimethylmethyloxy)phenyl)oxy]phenyl]methyl]-4-spirocyclopentyl-1H-imidazol-5(4H)-one (example 12.30). The product was chromatographed over silica gel (eluent dichloromethane/methanol 98/2). The product was obtained as a yellow powder.

Yield: 96%

Rf (dichloromethane/methanol 95/5): 0.30

IR: νCO 1730 cm⁻¹

NMR ¹H (CDCl₃): 0.87 (t, 3H, J=7.2 Hz); 1.31 (sext, 2H, J=7 Hz); 1.55 (m, 2H); 1.60 (s, 6H); 1.80-2.08 (m, 8H); 2.40 (t, 2H, J=8 Hz); 4.66 (s, 2H); 6.85-7.00 (m, 6H); 7.10 (d, 2H, J=8 Hz).

MS (ESI): 477 (M−H)

### Compound 35. 2-butyl-1-[[4-[(4-((1-carboxy-1,1-dimethylmethyloxy)phenyl)oxy]phenyl]methyl]-4-spirocyclohexyl-1H-imidazol-5(4H)-one

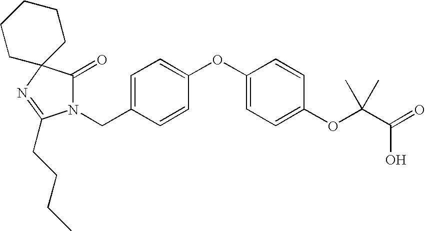

Prepared following the general procedure previously described (Method 15B) using 2-butyl-1-[[4-[(4-((1-tert-butyloxycarbonyl-1,1-dimethylmethyloxy)phenyl)oxy]phenyl]methyl]-4-spirocyclohexyl-1H-imidazol-5(4H)-one (example 12.31). The product was chromatographed over silica gel (eluent dichloromethane/methanol 97/3). The product was obtained as a yellow powder.

Yield: 70%

Rf (dichloromethane/methanol 95/5): 0.30

IR: νCO 1732 cm⁻¹

NMR ¹H (CDCl₃): 0.85 (t, 3H, J=7.2 Hz); 1.20-1.40 (m, 4H); 1.42-1.55 (m, 4H); 1.59 (s, 6H); 1.69-1.85 (m, 6H); 2.39 (t, 2H, J=8 Hz); 4.63 (s, 2H); 6.85-6.95 (m, 6H); 7.08 (d, 2H, J=8 Hz).

MS (ESI): 491 (M−H)

### Compound 36. 2-butyl-1-[[4-[(3-((1-carboxy-1,1-dimethylmethyloxy)phenyl)methyl]phenyl]methyl]-4-spirocyclopentyl-1H-imidazol-5(4H)-one

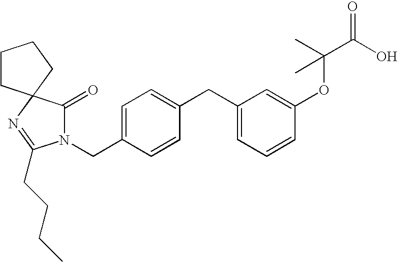

Prepared following the general procedure previously described (Method 15A) using 2-butyl-1-[[4-[(3-((1-ethoxycarbonyl-1,1-dimethylmethyloxy)phenyl)methyl]phenyl]methyl]-4-spirocyclopentyl-1H-imidazol-5(4H)-one (example 12.32). The product was chromatographed over silica gel (elution gradient dichloromethane/methanol 100/0 to 97/3). The product was obtained as a white solid.

Yield: 40%

Rf (dichloromethane/methanol 95/5): 0.30

IR: νCO: 1737 cm⁻¹

NMR ¹H (CDCl₃): 0.82 (t, 3H, J=7.2 Hz); 1.28 (sext, 2H, J=7 Hz); 1.52 (quint, 2H, J=7 Hz); 1.55 (s, 6H); 1.78-2.08 (m, 8H); 2.35 (t, 2H, J=8 Hz); 3.90 (s, 2H); 4.67 (s, 2H); 6.70 (s, 1H); 6.72 (d, 1H, J=8 Hz); 6.80 (d, 1H, J=8 Hz); 7.05 (d, 2H, J=8 Hz); 7.12 (m, 3H).

MS (ESI): 475 (M−H)

### Compound 37. 2-butyl-1-[[4-[(3-((1-carboxy-1,1-dimethylmethyloxy)phenyl)methyl]phenyl]methyl]-4-spirocyclohexyl-1H-imidazol-5(4H)-one

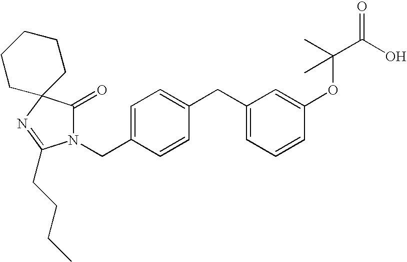

Prepared following the general procedure previously described (Method 15A) using 2-butyl-1-[[4-[(3-((1-ethoxycarbonyl-1,1-dimethylmethyloxy)phenyl)methyl]phenyl]methyl]-4-spirocyclohexyl-1H-imidazol-5(4H)-one (example 12.33). The product was chromatographed over silica gel (elution gradient dichloromethane/methanol 100/0 to 98/2). The product was obtained as a white solid.

Yield: 64%

Rf (dichloromethane/methanol 95/5): 0.30

IR: νCO: 1741 cm⁻¹

NMR ¹H (CDCl₃): 0.82 (t, 3H, J=7.2 Hz); 1.29 (m, 2H); 1.40-1.68 (m, 8H); 1.55 (s, 6H); 1.69-1.85 (m, 4H); 2.37 (t, 2H, J=8 Hz); 3.90 (s, 2H); 4.65 (s, 2H); 6.70 (s, 1H); 6.73 (d, 1H, J=8 Hz); 6.81 (d, 1H, J=8 Hz); 7.05 (d, 2H, J=8 Hz); 7.11 (m, 3H).

MS (ESI): 489 (M−H)

### Compound 38. 2-butyl-1-[(4′-((4-carboxy-4,4-dimethylbutan-1-yl)oxy)biphenyl-4-yl)methyl]-4-spirocyclohexyl-1H-imidazol-5(4H)-one

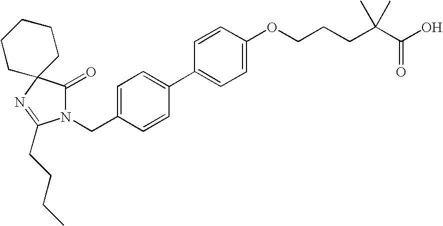

Prepared following the general procedure previously described (Method 15A) using 2-butyl-1-[(4′-((4-methoxycarbonyl-4,4-dimethylbutan-1-yl)oxy)biphenyl-4-yl)methyl]-4-spirocyclohexyl-1H-imidazol-5(4H)-one (example 12.34). The product was chromatographed over silica gel (eluent dichloromethane/methanol 95/5). The product was obtained as an amorphous solid.

Yield: 62%

Rf (dichloromethane/methanol 95/5): 0.30

IR: νCO: 1730 cm⁻¹

NMR ¹H (CDCl₃): 0.86 (t, 3H, J=7.2 Hz); 1.25 (s, 6H); 1.20-1.40 (m, 2H); 1.42-11.65 (m, 4H); 1.66-1.90 (m, 8H); 1.90-2.10 (m, 2H); 2.18 (t, 2H, J=8 Hz); 2.38 (t, 2H, 8 Hz); 3.98 (t, 2H, J=8 Hz); 4.70 (s, 2H); 6.93 (d, 2H, J=9.2 Hz); 7.18 (d, 1H, J=8 Hz); 7.48 (t, 4H, J=8.8 Hz).

MS (ESI): 517 (M−H)

### Compound 39. 2-butyl-1-[[4-[(3-((1-carboxy-1,1-dimethylmethyloxy)phenyl)oxy]phenyl]methyl]-4-spirocyclohexyl-1H-imidazol-5(4H)-one

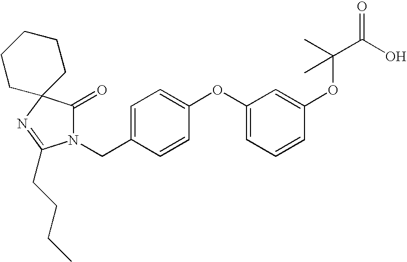

Prepared following the general procedure previously described (Method 15B) using 2-butyl-1-[[4-[(3-((1-tert-butyloxycarbonyl-1,1-dimethylmethyloxy)phenyl)oxy]phenyl]methyl]-4-spirocyclohexyl-1H-imidazol-5(4H)-one (example 12.35). The product was chromatographed over silica gel (eluent dichloromethane/methanol 98/2). The product was obtained as a colorless, very viscous oil.

Yield: 74%

Rf (dichloromethane/methanol 95/5): 0.30

IR: νCO: 1772 and 1732 cm⁻¹

NMR ¹H (CDCl₃): 0.91 (t, 3H, J=7.2 Hz); 1.38 (sext, 2H, J=7 Hz); 1.45-1.69 (m, 2H); 1.59 (s, 6H); 1.70-1.95 (m, 10H); 2.87 (t, 2H, J=8 Hz); 4.85 (s, 2H); 6.52 (s, 1H); 6.69 (d, 1H, J=8 Hz); 6.73 (d, 1H, J=8 Hz); 7.01 (d, 2H, J=8 Hz); 7.12 (d, 2H, J=8 Hz); 7.23 (t, 1H, J=8 Hz).

MS (ESI): 491 (M−H)

### Compound 40. 2-butyl-1-[[4-[(3-((1-carboxy-1,1-dimethylmethyloxy)phenyl)oxy]phenyl]methyl]-4-spirocyclopentyl-1H-imidazol-5(4H)-one

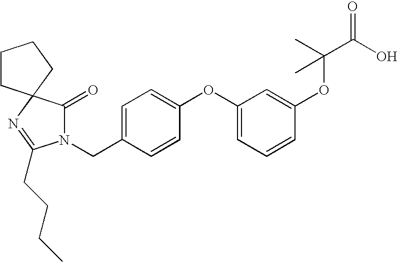

Prepared following the general procedure previously described (Method 15B) using 2-butyl-1-[[4-[(3-((1-tert-butyloxycarbonyl-1,1-dimethylmethyloxy)phenyl)oxy]phenyl]methyl]-4-spirocyclopentyl-1H-imidazol-5(4H)-one (example 12.36). The product was chromatographed over silica gel (eluent dichloromethane/methanol 98/2). The product was obtained as a colorless, very viscous oil.

Yield: 50%

Rf (dichloromethane/methanol 95/5): 0.30

IR: νCO: 1770 and 1735 cm⁻¹

NMR ¹H (CDCl₃): 0.86 (t, 3H, J=7.2 Hz); 1.34 (sext, 2H, J=7 Hz); 1.55-1.65 (m, 2H); 1.55 (s, 6H); 1.90-2.15 (m, 8H); 2.58 (t, 2H, J=8 Hz); 4.73 (s, 2H); 6.53 (s, 1H); 6.65 (dd, 2H, J=8 Hz, J=2 Hz); 6.97 (d, 2H, J=8 Hz); 7.10 (d, 2H, J=8 Hz); 7.19 (t, 1H, J=8 Hz).

MS (ESI): 477 (M−H)

### Compound 41. 2-butyl-1-[[4-[(2-((1-carboxy-1,1-dimethylmethyloxy)phenyl)methyl]phenyl]methyl]-4-spirocyclopentyl-1H-imidazol-5(4H)-one

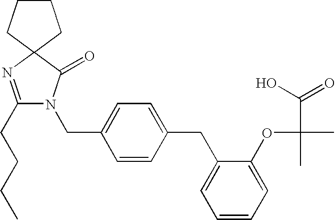

Prepared following the general procedure previously described (Method 15A) using 2-butyl-1-[[4-[(2-((1-ethoxycarbonyl-1,1-dimethylmethyloxy)phenyl)methyl]phenyl]methyl]-4-spirocyclopentyl-1H-imidazol-5(4H)-one (example 12.37). The product was chromatographed over silica gel (eluent dichloromethane/methanol 98/2). The product was obtained as a white solid.

Yield: 26%

Rf (dichloromethane/methanol 95/5): 0.30

IR: νCO: 1736 cm⁻¹

NMR ¹H (CDCl₃): 0.81 (t, 3H, J=7 Hz); 1.28 (sext, 2H, J=7 Hz); 1.50 (s, 6H); 1.45-1.60 (m, 2H); 1.80-2.08 (m, 8H); 2.38 (t, 2H, J=8 Hz); 3.95 (s, 2H); 4.67 (s, 2H); 6.77 (d, 1H, J=8 Hz); 6.90 (t, 1H, J=8 Hz); 7.03 (d, 2H, J=8 Hz); 7.10 (t, 2H, J=8 Hz); 7.18 (d, 2H, J=8 Hz).

MS (ESI): 475 (M−H)

### Compound 42. 2-butyl-1-[(2′-((4-carboxy-4,4-dimethylbutan-1-yl)oxy)biphenyl-4-yl)methyl]-4-spirocyclohexyl-1H-imidazol-5(4H)-one

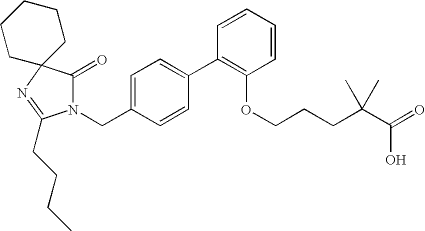

Prepared following the general procedure previously described (Method 15A) using 2-butyl-1-[(2′-((4-methoxycarbonyl-4,4-dimethylbutan-1-yl)oxy)biphenyl-4-yl)methyl]-4-spirocyclohexyl-1H-imidazol-5(4H)-one (example 12.38). The product was chromatographed over silica gel (eluent dichloromethane/methanol 99.5/0.5). The product was obtained as a white oil.

Yield: 63%

Rf (dichloromethane/methanol 95/5): 0.30

IR: νCO: 1734 cm⁻¹

NMR ¹H (CDCl₃): 0.87 (t, 3H, J=7 Hz); 1.16 (s, 6H); 1.20-1.45 (m, 8H); 1.49-1.90 (m, 10H) 2.38 (t, 2H, J=8 Hz); 3.91 (t, 2H, J=6 Hz); 4.73 (s, 2H); 6.94 (d, 1H, J=8 Hz); 7.02 (t, 1H, J=8 Hz); 7.15 (d, 2H, J=8 Hz); 7.29 (t, 2H, J=8 Hz); 7.49 (d, 2H, J=8 Hz).

MS (ESI): 517 (M−H)

### Compound 43. 2-butyl-1-[(2′-((7-carboxy-7,7-dimethylheptan-1-yl)oxy)biphenyl-4-yl)methyl]-4-spirocyclopentyl-1H-imidazol-5(4H)-one

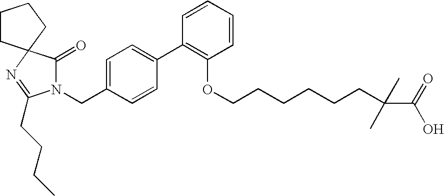

Prepared following the general procedure previously described (Method 15A) using 2-butyl-1-[(2′-((7-methoxycarbonyl-7,7-dimethylheptan-1-yl)oxy)biphenyl-4-yl)methyl]-4-spirocyclopentyl-1H-imidazol-5(4H)-one (example 12.39). The product was chromatographed over silica gel (eluent dichloromethane/methanol 99/1). The product was obtained as a colorless oil.

Yield: 6%

Rf (dichloromethane/methanol 95/5): 0.30

IR: νCO: 1730 cm⁻¹

NMR ¹H (CDCl₃): 0.87 (t, 3H, J=7 Hz); 1.16 (s, 6H); 1.11-1.40 (m, 8H); 1.41-1.72 (m, 6H); 1.85-2.10 (m, 8H); 2.35 (t, 2H, J=8 Hz); 3.92 (t, 2H, J=6 Hz); 4.79 (s, 2H); 6.94 (d, 1H, J=8 Hz); 7.01 (t, 1H, J=8 Hz); 7.13 (d, 2H, J=8 Hz); 7.27 (t, 2H, J=8 Hz); 7.47 (d, 2H, J=8 Hz).

MS (ESI): 545 (M−H)

### Compound 44. 2-butyl-1-[(2′-((4-carboxy-4,4-dimethylbutan-1-yl)oxy)biphenyl-4-yl)methyl]-4-spirocyclopentyl-1H-imidazol-5(4H)-one

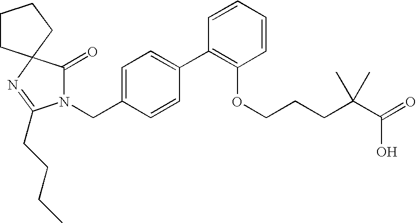

Prepared following the general procedure previously described (Method 15A) using 2-butyl-1-[(2′-((4-methoxycarbonyl-4,4-dimethylbutan-1-yl)oxy)biphenyl-4-yl)methyl]-4-spirocyclopentyl-1H-imidazol-5(4H)-one (example 12.40). The product was chromatographed over silica gel (eluent dichloromethane/methanol 99.5/0.5). The product was obtained as a white oil.

Yield: 54%

Rf (dichloromethane/methanol 95/5): 0.30

IR: νCO: 1736 cm⁻¹

NMR ¹H (CDCl₃): 0.87 (t, 3H, J=7 Hz); 1.16 (s, 6H); 1.35 (sext, 2H, J=7.2 Hz); 1.52-1.70 (m, 8H); 1.78-2.10 (m, 6H); 2.35 (t, 2H, J=8 Hz); 3.91 (t, 2H, J=6 Hz); 4.74 (s, 2H); 6.93 (d, 1H, J=8 Hz); 7.02 (t, 1H, J=8 Hz); 7.15 (d, 2H, J=8 Hz); 7.29 (t, 2H, J=8 Hz); 7.50 (d, 2H, J=8 Hz).

MS (ESI): 503 (M−H)

### Compound 45. 2-butyl-1-[[4-[(2-((1-carboxy-1,1-dimethylmethyloxy)phenyl)methyl]phenyl]methyl]-4-spirocyclohexyl-1H-imidazol-5(4H)-one

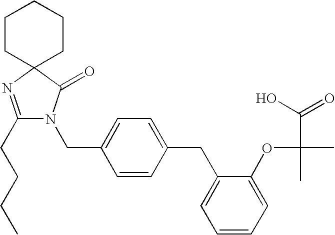

Prepared following the general procedure previously described (Method 15A) using 2-butyl-1-[[4-[(2-((1-ethoxycarbonyl-1,1-dimethylmethyloxy)phenyl)methyl]phenyl]methyl]-4-spirocyclohexyl-1H-imidazol-5(4H)-one (example 12.41). The product was chromatographed over silica gel (eluent dichloromethane/methanol 97/3); The product was obtained as a yellow solid.

Yield: 18%

Rf (dichloromethane/methanol 95/5): 0.30

IR: νCO: 1742 cm⁻¹

NMR ¹H (CDCl₃): 0.85 (t, 3H, J=7 Hz); 1.31-1.45 (m, 2H); 1.50-1.70 (m, 4H); 1.52 (s, 6H); 1.71-2.00 (m, 8H); 2.05 (t, 2H, J=8 Hz); 3.95 (s, 2H); 4.74 (s, 2H); 6.72 (d, 1H, J=9 Hz); 6.92 (t, 1H, J=8 Hz); 7.02 (d, 2H, J=8 Hz); 7.11 (t, 2H, J=8 Hz); 7.19 (d, 2H, J=8 Hz).

MS (ESI): 489 (M−H)

### Compound 46. 2-butyl-1-[[4-[(3-((1-carboxy-1,1-dimethylmethyloxy)phenyl)thio]phenyl]methyl]-4-spirocyclopentyl-1H-imidazol-5(4H)-one

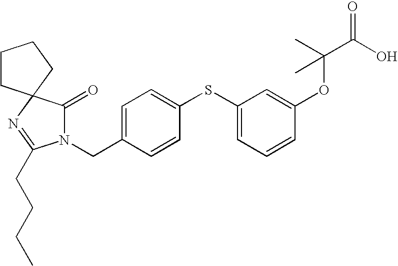

Prepared following the general procedure previously described (Method 15B) using 2-butyl-1-[[4-[(3-((1-tert-butyloxycarbonyl-1,1-dimethylmethyloxy)phenyl)thio]phenyl]methyl]-4-spirocyclopentyl-1H-imidazol-5(4H)-one (example 12.42). The product was chromatographed over silica gel (eluent dichloromethane/methanol 98/2). The product was obtained as an amorphous yellow solid.

Yield: 26%

Rf (dichloromethane/methanol 95/5): 0.30

IR: νCO: 1738 cm⁻¹

NMR ¹H (CDCl₃): 0.86 (t, 3H, J=7.2 Hz); 1.20-1.45 (m, 2H); 1.45-1.70 (m, 2H); 1.55 (s, 6H); 1.75-2.10 (m, 8H); 2.39 (t, 2H, J=6 Hz); 4.69 (s, 2H); 6.62 (d, 1H, J=2 Hz); 6.75 (dd, 1H, J=8 Hz, J=2 Hz); 6.95 (d, 1H, J=8 Hz); 7.02-7.20 (m, 3H); 7.35 (d, 2H, J=8 Hz).

MS (ESI): 493 (M−H)

### Compound 47. 2-butyl-1-[[4-[(3-((1-carboxy-1,1-dimethylmethyloxy)phenyl)thio]phenyl]methyl]-4-spirocyclohexyl-1H-imidazol-5(4H)-one

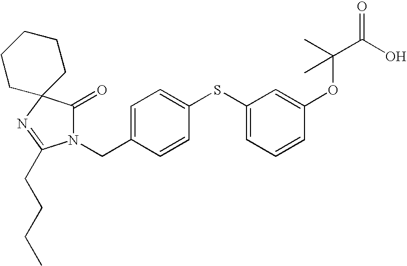

Prepared following the general procedure previously described (Method 15B) using 2-butyl-1-[[4-[(3-((1-tert-butyloxycarbonyl-1,1-dimethylmethyloxy)phenyl)thio]phenyl]methyl]-4-spirocyclohexyl-1H-imidazol-5(4H)-one (example 12.43). The product was chromatographed over silica gel (eluent dichloromethane/methanol 99/1). The product was obtained as a beige solid.

Yield: 80%

Rf (dichloromethane/methanol 95/5): 0.30

IR: νCO: 1738 cm⁻¹

NMR ¹H (CDCl₃): 0.90 (t, 3H, J=7.2 Hz); 1.20-1.45 (m, 4H); 1.53 (s, 6H); 1.55-2.00 (m, 10H); 2.86 (t, 2H, J=6 Hz); 4.82 (s, 2H); 6.72 (d, 1H, J=2 Hz); 6.82 (dd, 1H, J=8 Hz, J=2 Hz); 7.08 (d, 1H, J=8 Hz); 7.15-7.25 (m, 3H); 7.32 (d, 2H, J=8 Hz).

MS (ESI): 507 (M−H)

### Compound 48. 2-butyl-1-[[4-[(4-((1-carboxy-1,1-dimethylmethyloxy)phenyl)thio]phenyl]methyl]-4-spirocyclohexyl-1H-imidazol-5(4H)-one

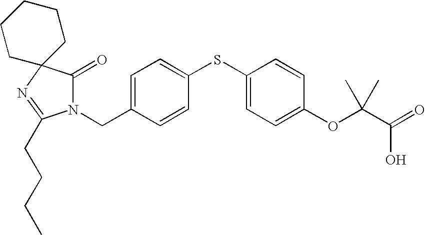

Prepared following the general procedure previously described (Method 15B) using 2-butyl-1-[[4-[(4-((1-tert-butyloxycarbonyl-1,1-dimethylmethyloxy)phenyl)thio]phenyl]methyl]-4-spirocyclohexyl-1H-imidazol-5(4H)-one (example 12.44). The product was chromatographed over silica gel (elution gradient dichloromethane/methanol 100/0 to 99/1). The product was obtained as a white solid.

Yield: 16%

Rf (dichloromethane/methanol 95/5): 0.30

IR: νCO 1730 cm⁻¹

NMR ¹H (CDCl₃): 0.82 (t, 3H, J=7.2 Hz); 1.10-1.40 (m, 4H); 1.41-1.85 (m, 10H); 1.62 (s, 6H); 2.39 (t, 2H, J=8 Hz); 4.62 (s, 2H); 6.90 (d, 2H, J=8 Hz); 6.99 (d, 2H, J=8 Hz); 7.11 (d, 2H, J=8 Hz); 7.31(d, 2H, J=8 Hz).

MS (ESI): 507 (M−H)

### Compound 49. 2-butyl-1-[(3′-((2-carboxy-2,2-dimethylethyl-1-yl)oxy)biphenyl-4-yl)methyl]-4-spirocyclopentyl-1H-imidazol-5(4H)-one

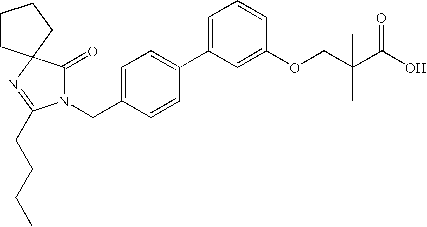

Prepared following the general procedure previously described (Method 15A) using 2-butyl-1-[(3′-((2-methoxycarbonyl-2,2-dimethylethyl-1-yl)oxy)biphenyl-4-yl)methyl]-4-spirocyclopentyl-1H-imidazol-5(4H)-one (example 12.45). The product was chromatographed over silica gel (eluent dichloromethane/methanol 99/1). The product was obtained as a white solid.

Yield: 40%

Rf (dichloromethane/methanol 95/5): 0.30

IR: νCO: 1730 cm⁻¹

NMR ¹H (CDCl₃+2 drops of CD₃OD): 0.87 (t, 3H, J=7 Hz); 1.19 (s, 6H); 1.20-1.47 (m, 4H); 1.48-1.65 (m, 2H); 1.80-2.12 (m, 6H); 2.44 (t, 2H, J=8 Hz); 3.97 (s, 2H); 4.77 (s, 2H); 7.00 (m, 2H); 7.02 (d, 2H, J=8 Hz); 7.28 (d, 2H, J=8 Hz); 7.50 (d, 2H, J=8 Hz).

MS (ESI): 475 (M−H)

### Compound 50. 2-butyl-1-[[4-[(4-((1-carboxy-1,1-dimethylmethyloxy)phenyl)methyl]phenyl]methyl]-4-spirocyclohexyl-1H-imidazol-5(4H)-one

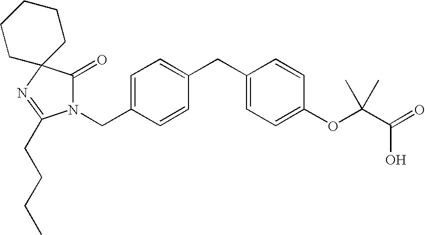

Prepared following the general procedure previously described (Method 15A) using 2-butyl-1-[[4-[(4-((1-ethoxycarbonyl-1,1-dimethylmethyloxy)phenyl)methyl]phenyl]methyl]-4-spirocyclohexyl-1H-imidazol-5(4H)-one (example 12.46). The product was chromatographed over silica gel (elution gradient dichloromethane/methanol 100/0 to 98/2). The product was obtained as a yellowish powder.

Yield: 10%

Rf (dichloromethane/ethyl acetate 95/5): 0.50

IR: νCO: 1733 cm⁻¹

NMR ¹H (CDCl₃): 0.83 (t, 3H, J=7.3 Hz); 1.28 (sext, 2H, J=7.6 Hz); 1.52 (quint, 2H, J=7.6 Hz); 1.60 (s, 6H); 1.55-1.86 (m, 10H); 2.38 (t, 2H, J=7.6 Hz); 3.91 (s, 2H); 4.66 (s, 2H); 6.86 (d, 2H, J=8.5 Hz); 7.03-7.11 (m, 4H); 7.13 (d, 2H, J=8.2 Hz).

MS (ESI): 489 (M−H)

### Compound 51. 2-butyl-1-[[4-[(4-((1-carboxy-1,1-dimethylmethyloxy)phenyl)thio]phenyl]methyl]-4-spirocyclopentyl-1H-imidazol-5(4H)-one

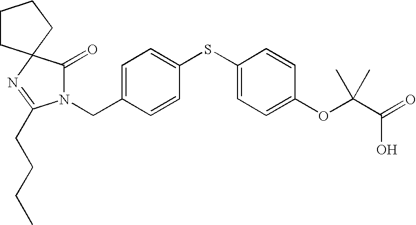

Prepared following the general procedure previously described (Method 15B) using 2-butyl-1-[[4-[(4-((1-tert-butyloxycarbonyl-1,1-dimethylmethyloxy)phenyl)thio]phenyl]methyl]-4-spirocyclopentyl-1H-imidazol-5(4H)-one (example 12.47). The product was chromatographed over silica gel (elution gradient dichloromethane/methanol 100/0 to 98/2). The product was obtained as a beige solid.

Yield: 70%

Rf (dichloromethane/methanol 95/5): 0.32

IR: νCO 1732 cm⁻¹

NMR ¹H (CDCl₃): 0.82 (t, 3H, J=7.2 Hz); 1.29 (sext, 2H, J=7 Hz); 1.52 (quint, 2H, J=7 Hz); 1.62 (s, 6H); 1.82-2.08 (m, 8H); 2.45 (t, 2H, J=8 Hz); 4.66 (s, 2H); 6.88 (dd, 2H, J=8 Hz, J=2 Hz); 7.01 (d, 2H, J=8 Hz); 7.13 (dd, 2H, J=8 Hz, J=2 Hz); 7.32 (d, 2H, J=8 Hz).

MS (ESI): 493 (M−H)

### Compound 52. 2-butyl-1-[(3′-((1-carboxymethyl)oxy)biphenyl-4-yl)methyl]-4-spirocyclohexyl-1H-imidazol-5(4H)-one

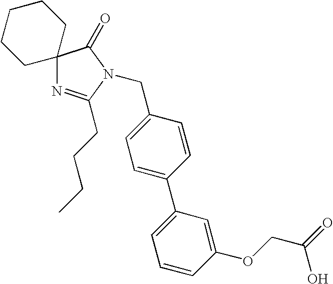

Prepared following the general procedure previously described (Method 15A) using 2-butyl-1-[(3′-((1-ethoxycarbonylmethyl)oxy)biphenyl-4-yl)methyl]-4-spirocyclohexyl-1H-imidazol-5(4H)-one (example 14.1). The product was obtained as a white solid.

Yield: 94.4%

Rf (dichloromethane/methanol 90/10): 0.35

IR: νCO 1731 cm⁻¹

MP: 82-85° C.

NMR ¹H (CDCl₃): 0.81 (t, 3H, J=7.29 Hz9); 1.28 (m, 2H); 1.56 (m, 12H); 2.43 (t, 2H, J=7.9 Hz); 4.71 (m, 4H); 6.95 (dd, 1H, J=8.2 Hz, J=1.8 Hz); 7.09 (m, 1H); 7.18 (m, 3H); 7.37 (t, 2H, J=7.9 Hz); 7.53 (d, 2H, J=8.2 Hz).

MS (ESI): 447 (M−H)

### Compound 53. 2-butyl-1-[(3′-((1-carboxy-1-methylmethyl)oxy)biphenyl-4-yl)methyl]-4-spirocyclopentyl-1H-imidazol-5(4H)-one

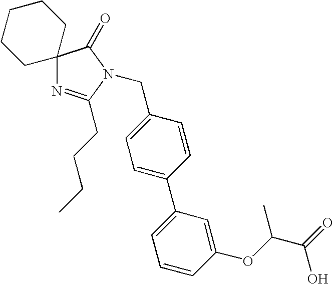

Prepared following the general procedure previously described (Method 15A) using 2-butyl-1-[(3′-((1-ethoxycarbonyl-1-methylmethyl)oxy)biphenyl-4-yl)methyl]-4-spirocyclohexyl-1H-imidazol-5(4H)-one (example 14.2). The product was obtained as a white solid.

Yield: 87%

Rf (dichloromethane/methanol 90/10): 0.35

IR: νCO 1729 cm⁻¹

MP: 85° C.

NMR ¹H (CDCl₃): 0.74 (t, 3H, J=7.23 Hz); 1.21 (m 2H); 1.57 (m, 15H); 2.43 (t, 2H, J=7.9 Hz); 4.69 (q, 2H); 4.82 (q, 1H, J=6.7 Hz); 6.94 (dd, 1H); 7.09 (m, 1H); 7.15 (m, 3H); 7.34 (t, 1H, J=8.2 Hz); 7.51 (d, 2H, J=7.9 Hz).

MS (ESI): 461 (M−H)

### Compound 54. 2-butyl-1-[(3′-((1-carboxy-1-ethylmethyl)oxy)biphenyl-4-yl)methyl]-4-spirocyclohexyl-1H-imidazol-5(4H)-one

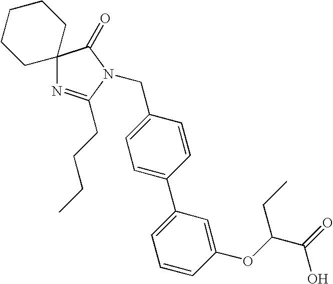

Prepared following the general procedure previously described (Method 15A) using 2-butyl-1-[(3′-((1-ethoxycarbonyl-1-ethylmethyl)oxy)biphenyl-4-yl)methyl]-4-spirocyclohexyl-1H-imidazol-5(4H)-one (example 14.3). The product was obtained as a white solid.

Yield: 64%

Rf (dichloromethane/methanol 90/10): 0.35

MP: 170° C.

IR: νCO 1728 cm⁻¹

NMR ¹H (CDCl₃): 0.72 (t, 3H, J=7.3 Hz); 1.15 (m, 5H); 1.55 (m, 12H); 2.07 (m, 2H); 2.39 (m, 2H); 4.67 (m, 3H); 6.96 (dd, 1H, J=8.2 Hz); 7.1 (m, 1H); 7.15 (m, 3H); 7.34 (t, 1H, J=7.9 Hz); 7.51 (d, 2H, J=8.2 Hz).

MS (ESI): 475 (M−H)

### Compound 55. 2-butyl-1-[(3′-((1-carboxy-1-(1,1-dimethylmethyl)methyl)oxy)-biphenyl-4-yl)methyl]-4-spirocyclohexyl-1H-imidazol-5(4H)-one

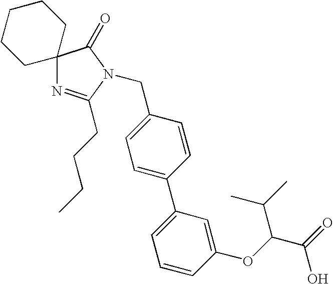

Prepared following the general procedure previously described (Method 15A) using 2-butyl-1-[(3′-((1-ethoxycarbonyl-1-(1,1-dimethylmethyl)methyl)oxy)biphenyl-4-yl)methyl]-4-spirocyclohexyl-1H-imidazol-5(4H)-one (example 14.4). The product was obtained as a white solid.

Yield: 89.4%

Rf (dichloromethane/methanol 90/10): 0.4

MP: 105° C.

IR: νCO 1733, 1772 cm⁻¹

NMR ¹H (CDCl₃): 0.79 (t, 3H, J=7.3 Hz); 1.13 (d, 6H, J=6.7 Hz); 1.29 (m, 2H); 1.52 (m, 3H); 1.84 (m, 9H); 2.34 (m, 1H); 2.75 (m, 2H); 4.47 (d, 1H, J=5.3 Hz); 4.79 (s, 2H); 6.91 (dd, 1H, J=8.2 Hz); 7.15 (m, 4H); 7.34 (t, 1H, J=7.Hz9); 7.54 (d, 2H, J=7.9 Hz).

MS (ESI): 489 (M−H)

### Compound 56. 2-butyl-1-[(3′-((1-carbonylmethyl)oxy)-6′-fluoro-biphenyl-4-yl)methyl]-4-spirocyclohexyl-1H-imidazol-5(4H)-one

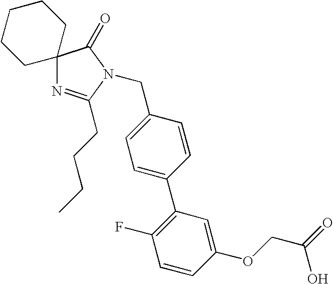

Prepared following the general procedure previously described (Method 15A) using 2-butyl-1-[(3′-((1-ethoxycarbonylmethyl)oxy)-6′-fluoro-biphenyl-4-yl)methyl]-4-spirocyclohexyl-1H-imidazol-5(4H)-one (example 14.5). The product was obtained as a white solid.

Yield: 68.5%

Rf (dichloromethane/methanol 90/10): 0.35

MP: 102-105° C.

IR: νCO 1729 cm⁻¹

NMR ¹H (CDCl₃): 0.86 (t, 3H, J=7.3 Hz); 1.39 (m, 14H); 2.73 (m, 2H); 4.67 (s, 2H); 4.82 (s, 2H); 6.92 (m, 2H); 7.09 (t, 1H, J=9.4 Hz); 7.21 (d, 2H, J=8.2 Hz); 7.52 (d, 2H, J=7.3 Hz).

MS (ESI): 467 (M+H)

### Compound 57. 2-butyl-1-[(3′-((1-carboxy-1-methylmethyl)oxy)-6′-fluoro-biphenyl-4-yl)methyl]-4-spirocyclohexyl-1H-imidazol-5(4H)-one

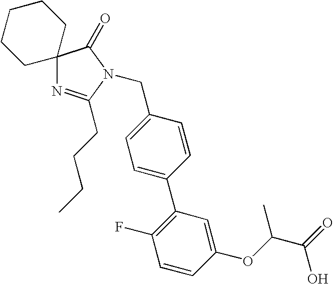

Prepared following the general procedure previously described (Method 15A) using 2-butyl-1-[(3′-((1-ethoxycarbonyl-1-methylmethyl)oxy)-6′-fluoro-biphenyl-4-yl)methyl]-4-spirocyclohexyl-1H-imidazol-5(4H)-one (example 14.6). The product was obtained as a white solid.

Yield: 60.5%

Rf (dichloromethane/methanol 90/10): 0.25

MP: 96° C.

IR: νCO 1733 cm⁻¹

NMR ¹H (CDCl₃): 0.78 (t, 3, J=7.32); 1.27-1.79 (m, 18); 2.53 (m, 2); 4.77 (m, 2); 6.92 (m, 2); 7.08 (t, 1, J=9.63); 7.18 (d, 2, J=8.19); 7.5 (d, 2, J=7.29).

MS (ESI): 481 (M+H)

### Compound 58. 2-butyl-1-[(3′-((1-carboxy-1-ethylmethyl)oxy)-6′-fluoro-biphenyl-4-yl)methyl]-4-spirocyclohexyl-1H-imidazol-5(4H)-one

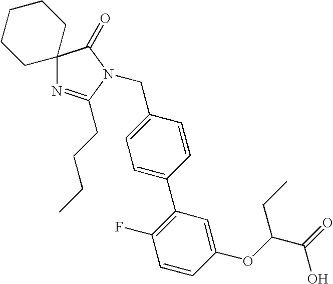

Prepared following the general procedure previously described (Method 12A) using 2-butyl-1-[(3′-((1-ethoxycarbonyl-1-ethylmethyl)oxy)-6′-fluoro-biphenyl-4-yl)methyl]-4-spirocyclohexyl-1H-imidazol-5(4H)-one (example 10.54). The product was obtained as a white solid.

Yield: 75.6%

Rf (dichloromethane/methanol 90/10): 0.35

IR: νCO 1731 cm⁻¹

NMR ¹H (CDCl₃): 0.8 (t, 3H, J=7.3 Hz); 1.13 (t, 3H, J=7.3 Hz); 1.27 (m, 2H); 1.65 (m, 12H); 2.05 (t, 2H, J=7 Hz); 2.57 (m, 2H); 4.58 (t, 1H, J=5.9 Hz); 4.76 (s, 2H); 6.92 (m, 2H); 7.07 (t, 1H, J=9.7 Hz); 7.18 (d, 2H, J=7.9 Hz); 7.5 (d, 2H, J=7.3 Hz).

MS (ESI): 495 (M+H)

### Compound 59. 2-butyl-1-[(3′-((1-carboxy-1-(1,1-dimethylmethyl)-methyl)oxy)-6′-fluoro-biphenyl-4-yl)methyl]-4-spirocyclohexyl-1H-imidazol-5(4H)-one

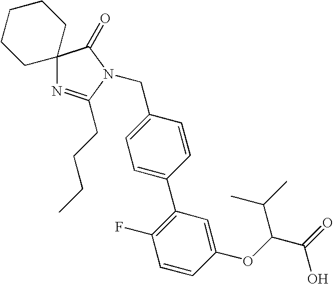

Prepared following the general procedure previously described (Method 15A) using 2-butyl-1-[(3′-((1-ethoxycarbonyl-1-(1,1-dimethylmethyl)methyl)oxy)-6′-fluoro-biphenyl-4-yl)methyl]-4-spirocyclohexyl-1H-imidazol-5(4H)-one (example 14.8). The product was obtained as a white solid.

Yield: 80.5%

Rf (dichloromethane/methanol 90/10): 0.35

MP: 95° C.

IR: νCO 1732 cm⁻¹

NMR ¹H (CDCl₃): 0.76 (t, 3H, J=7.3 Hz); 1.14 (m, 6H); 1.24 (m, 2H); 1.6 (m, 12H); 2.32 (m, 1H); 2.49 (m, 2H); 4.37 (d, 1H, J=7.6 Hz); 4.73 (m, 2H); 6.92 (m, 2H); 7.07 (t, 1H, J=9.1 Hz); 7.18 (d, 2H, J=8.2 Hz); 7.49 (d, 2H, J=7.3 Hz).

MS (ESI): 509 (M+H)

### Compound 60. 2-butyl-1-[(3′-((1-carboxy-1,1-dimethylmethyl)oxy)-6′-fluoro-biphenyl-4-yl)methyl]-4-spirocyclohexyl-1H-imidazol-5(4H)-one

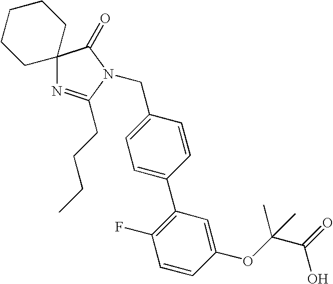

Prepared following the general procedure previously described (Method 15A) using 2-butyl-1-[(3′-((1-ethoxycarbonyl-1,1-dimethylmethyl)oxy)-6′-fluoro-biphenyl-4-yl)methyl]-4-spirocyclohexyl-1H-imidazol-5(4H)-one (example 14.9). The product was obtained as a white solid.

Yield: 45.5%

Rf (dichloromethane/methanol 90/10): 0.25

MP: 72° C.

IR: νCO 1730 cm⁻¹

NMR ¹H (DMSO): 0.79 (t, 3H, J=7.3 Hz); 1.44 (m, 20H); 2.35 (t, 2H, J=7.6 Hz); 4.72 (s, 2H); 6.87 (m, 1H); 6.94 (m, 1H); 7.22 (m, 3H); 7.49 (d, 2H, J=7.6 Hz); 13.13 (s, 1H).

MS (ESI): 495 (M+H)

### Compound 61. 2-butyl-4-spirocyclohexyl-1-[(3′-((1-(tetrazol-5-yl)methyl)oxy)-biphenyl-4-yl)methyl]-1H-imidazol-5(4H)-one

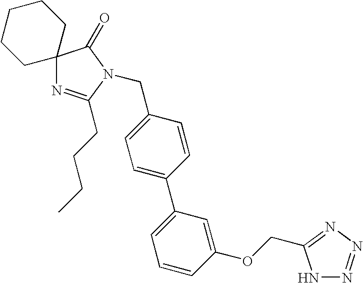

Prepared following the general procedure previously described (Method 15E) using 2-butyl-1-[(3′-((1-cyanomethyl)oxy)-biphenyl-4-yl)methyl]-4-spirocyclohexyl-1H-imidazol-5(4H)-one (example 14.10). The product was chromatographed over silica gel (elution gradient dichloromethane/methanol 95/5 to 90/10); The product was obtained as a white solid.

Yield: 36.4%

Rf (dichloromethane/methanol 90/10): 0.2

MP: 76° C.

IR: νCO 1721 cm⁻¹

NMR ¹H (CDCl₃): 0.78 (t, 3H, J=7.3 Hz); 0.87 (m, 2H); 1.51 (m, 12H); 2.38 (t, 2H, J=8.2 Hz); 4.73 (s, 2H); 5.49 (s, 2H); 6.94 (dd, 1H, J=8.2 Hz, J=1.9 Hz); 7.11 (m, 1H); 7.16 (m, 3H); 7.34 (t, 1H, J=7.9 Hz); 7.44 (d, 2H, J=7.9 Hz).

MS (ESI): 471 (M+H)

### Compound 62. 2-butyl-1-[(6′-fluoro-3′-((1-(tetrazol-5-yl)methyl)oxy)biphenyl-4-yl)methyl]-4-spirocyclohexyl-1H-imidazol-5(4H)-one

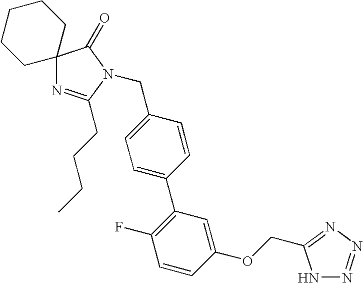

Prepared following the general procedure previously described (Method 15E) using 2-butyl-1-[(6′-fluoro-3′-((1-cyanomethyl)oxy)-biphenyl-4-yl)methyl]-4-spirocyclohexyl-1H-imidazol-5(4H)-one (example 14.11). The product was chromatographed over silica gel (elution gradient dichloromethane/methanol 95/5 to 90/10). The product was obtained as a yellow solid.

Yield: 63.9%

Rf (dichloromethane/methanol 90/10): 0.2

IR: νCO 1720 cm⁻¹

NMR ¹H (CDCl₃): 0.8 (m, 3H); 1.48 (m, 14H); 2.36 (t, 2H, J=7.3 Hz); 4.73 (s, 2H); 5.42 (s, 2H); 7.06 (m, 1H); 7.23 (m, 4H); 7.76 (d, 2H, J=7.6 Hz).

MS (ESI): 491 (M+H)

### Compound 63. 2-butyl-1-[(3′-((1-carboxy-1-(1,1-dimethylmethyl)methyl)oxy)-biphenyl-4-yl)methyl]-4,4-diethyl-1H-imidazol-5(4H)-one

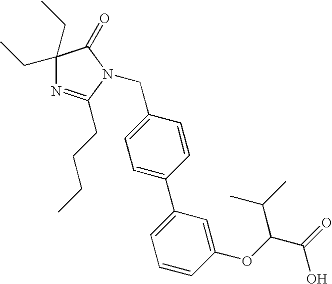

Prepared following the general procedure previously described (Method 15A) using 2-butyl-1-[(3′-((1-ethoxycarbonyl-1-(1,1-dimethylmethyl)methyl)oxy)-biphenyl-4-yl)methyl]-4,4-diethyl-1H-imidazol-5(4H)one (example 14.12). The product was chromatographed over silica gel (eluent dichloromethane/methanol 95/5). The product was obtained as a white solid.

Yield: 78.2%

Rf (dichloromethane/methanol 95/5): 0.3

MP: 65° C.

IR: νCO 1736 cm⁻¹

NMR ¹H (DMSO): 0.63 (t, 6H, J=7 Hz); 0.81 (t, 3H, J=7.3 Hz); 1.03 (d, 6H, J=6.7 Hz); 1.32 (m, 2H); 1.53 (m, 2H); 1.66 (m, 4H); 2.21 (m, 1H); 2.42 (t, 2H, J=7.3 Hz); 4.59 (d, 1H, J=4.4 Hz); 4.71 (s, 2H); 6.86 (dd, 1, J=7.2 Hz, J=1.8 Hz); 7.13 (m, 1H); 7.23 (d, 1H, J=7.9 Hz); 7.32 (m, 3H); 7.62 (d, 2H, J=8.2 Hz); 13.07 (s, 1H).

MS (ESI): 479 (M+H)

### Compound 64. 2-butyl-1-[(3′-((1-carboxy-1-ethylmethyl)oxy)biphenyl-4-yl)methyl]-4,4-diethyl-1H-imidazol-5(4H)one

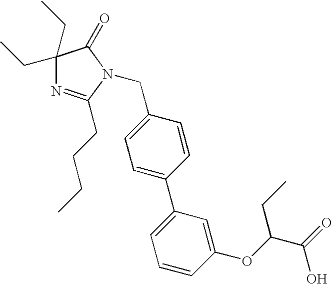

Prepared following the general procedure previously described (Method 15A) using 2-butyl-1-[(3′-((1-ethoxycarbonyl-1-ethylmethyl)oxy)biphenyl-4-yl)methyl]-4,4-diethyl-1H-imidazol-5(4H)-one (example 14.13). The product was chromatographed over silica gel (eluent dichloromethane/methanol 95/5). The product was obtained as a white solid.

Yield: 38.3%

Rf (dichloromethane/methanol 95/5): 0.3

MP: 184° C.

IR: νCO 1739 cm⁻¹

NMR ¹H (DMSO): 0.63 (t, 6H, J=7.3 Hz); 0.81 (t, 3H, J=7.3 Hz); 1.03 (t, 3H, J=7.3 Hz); 1.32 (m, 2H); 1.53 (m, 2H); 1.64 (m, 4H); 1.88 (m, 2H); 2.42 (t, 2H, J=7.3 hZ); 4.71 (s, 2H); 4.75 (t, 1H, J=5.6 Hz); 6.85 (dd, 1H, J=7.6 Hz, J=1.7 Hz); 7.13 (m, 1H); 7.23 (d, 1H, J=7.9 Hz); 7.32 (m, 3H); 7.65 (d, 2J, J=8.2 Hz); 13.08 (s, 1H).

MS (ESI): 465 (M+H)

### Compound 65. 2-butyl-1-[(3′-((1-carboxy-1,1-dimethylmethyl)oxy)biphenyl-4-yl)methyl]-4,4-diethyl-1H-imidazol-5(4H)one

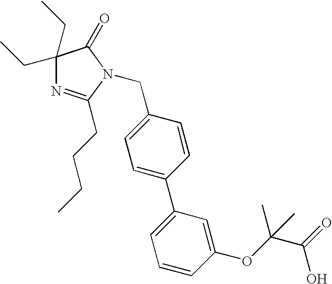

Prepared following the general procedure previously described (Method 15A) using 2-butyl-1-[(3′-((1-ethoxycarbonyl-1,1-dimethylmethyl)oxy)biphenyl-4-yl)methyl]-4,4-diethyl-1H-imidazol-5(4H)-one (example 14.14). The product was chromatographed over silica gel (eluent dichloromethane/methanol 95/5). The product was obtained as a white solid.

Yield: 86.8%

Rf (dichloromethane/methanol 90/10): 0.3

MP: 144° C.

IR: νCO 1715 cm⁻¹

NMR ¹H (DMSO): 0.63 (t, 6H, J=7 Hz); 0.81 (t, 3H, J=7.3 Hz); 1.29 (m, 2H); 1.53 (m, 8H); 1.64 (m, 4H); 2.42 (t, 2H, J=7.3 Hz); 4.71 (s, 2H); 6.81 (dd, 1H, J=7.6 Hz); 7.07 (m, 1H); 7.31 (m, 4H); 7.59 (d, 2H, J=8.19); 13.11 (s, 1H).

MS (ESI): 465 (M+H)

### Compound 66. 2-butyl-1-[(3′-((1-carboxy-1-methylmethyl)oxy)biphenyl-4-yl)methyl]-4,4-diethyl-1H-imidazol-5(4H)one

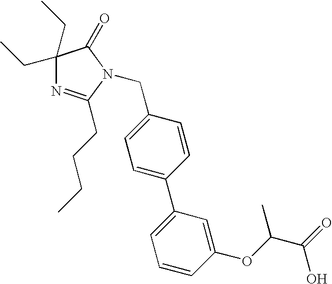

Prepared following the general procedure previously described (Method 15A) using 2-butyl-1-[(3′-((1-ethoxycarbonyl-1-methylmethyl)oxy)biphenyl-4-yl)methyl]-4,4-diethyl-1H-imidazol-5(4H)-one (example 14.15). The product was chromatographed over silica gel (eluent dichloromethane/methanol 95/5). The product was obtained as a white solid.

Yield: 56.3%

Rf (dichloromethane/methanol 90/10): 0.3

MP: 186° C.

IR: νCO 1723 cm⁻¹

NMR ¹H (DMSO): 0.64 (t, 6H, J=7.3 Hz2); 0.81 (t, 3H, J=7.3 Hz); 1.31 (m, 2H); 1.53 (m, 5H); 1.67 (m, 4H); 2.45 (m, 2H); 4.72 (s, 2H); 4.96 (q, 1H, J=6.7 Hz); 6.85 (dd, 1H, J=7.9 Hz, J=1.7 Hz); 7.13 (m, 1H); 7.23 (d, 1H, J=7.9 Hz); 7.32 (m, 3); 7.64 (d, 2H, J=7.9 Hz); 13.02 (s, 1).

MS (ESI): 451 (M+H)

### Compound 67. 2-butyl-1-[(3′-((1-carboxy-1-spirocyclobutylmethyl)oxy)biphenyl-4-yl)methyl]-4-spirocyclohexyl-1H-imidazol-5(4H)-one

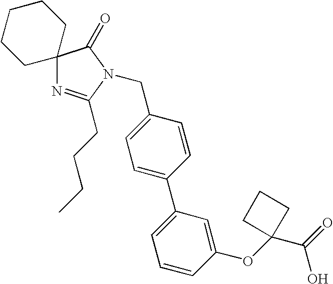

Prepared following the general procedure previously described (Method 15A) using 2-butyl-1-[(3′-((1-ethoxycarbonyl-1-spirocyclobutylmethyl)oxy)biphenyl-4-yl)methyl]-4-spirocyclohexyl-1H-imidazol-5(4H)-one (example 14.16). The product was chromatographed over silica gel (eluent dichloromethane/methanol 95/5). The product was obtained as a white solid.

Yield: 45.3%

Rf (dichloromethane/methanol 90/10): 0.4

MP: 184° C.

IR: νCO 1728 cm⁻¹

NMR ¹H (DMSO): 0.79 (t, 3H, J=7.3 Hz); 1.38 (m, 14H); 1.93 (m, 2H); 2.35 (m, 4H); 2.69 (m, 2H); 4.71 (s, 2H); 6.61 (dd, 1H, J=7.6 Hz, J=1.7 Hz); 6.9 (m, 1H); 7.2 (m, 3H); 7.33 (t, 1H, J=7.9 Hz); 7.58 (d, 2H, J=8.2 Hz).

MS (ESI): 487 (M−H)

### Compound 68. 2-butyl-1-[(3′-((1-carboxy-1-(1,1-dimethylmethyl)methyl)oxy)-6′-fluoro-biphenyl-4-yl)methyl]-4,4-diethyl-1H-imidazol-5(4H)one

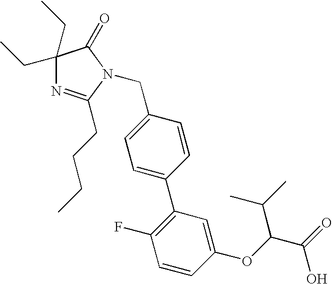

Prepared following the general procedure previously described (Method 15A) using 2-butyl-1-[(3′-((1-ethoxycarbonyl-1-(1,1-dimethylmethyl)methyl)oxy)-6′-fluoro-biphenyl-4-yl)methyl]-4,4-diethyl-1H-imidazol-5(4H)one (example 14.17). The product was chromatographed over silica gel (eluent dichloromethane/methanol 95/5). The product was obtained as a white solid.

Yield: 49.3%

Rf (dichloromethane/methanol 90/10): 0.4

MP: 65° C.

IR: νCO 1734 cm⁻¹

NMR ¹H (DMSO): 0.64 (t, 6H, J=7 Hz); 0.81 (t, 3H, J=7.3 Hz); 1.01 (d, 6H, J=6.7 Hz); 1.29 (m, 2H); 1.53 (m, 2H); 1.65 (m, 4H); 2.21 (m, 1H); 2.43 (t, 2H, J=7.6 Hz); 4.54 (d, 1H, J=4.7 Hz); 4.72 (s, 2H); 6.89 (m, 1H); 6.95 (m, 1H); 7.21 (m, 1H); 7.31 (d, 2H, J=8.2 Hz); 7.53 (d, 2H, J=7 Hz).

MS (ESI): 495 (M−H)

### Compound 69. 2-butyl-1-[(3′-((1-carboxy-1,1-dimethylmethyl)oxy)-6′-fluoro-biphenyl-4-yl)methyl]-4,4-diethyl-1H-imidazol-5(4Hone

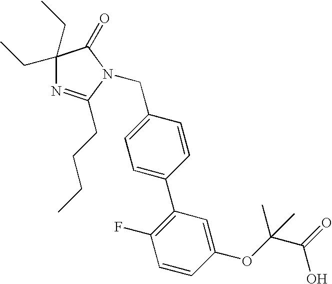

Prepared following the general procedure previously described (Method 15A) using 2-butyl-1-[(3′-((1-ethoxycarbonyl-1,1-dimethylmethyl)oxy)-6′-fluoro-biphenyl-4-yl)methyl]-4,4-diethyl-1H-imidazol-5(4H)one (example 14.18). The product was chromatographed over silica gel (eluent dichloromethane/methanol 95/5). The product was obtained as a white solid.

Yield: 63.5%

Rf (dichloromethane/methanol 90/10): 0.4

MP: 62° C.

IR: νCO 1736 cm⁻¹

NMR ¹H (DMSO): 0.63 (t, 6H, J=7.6 Hz); 0.81 (t, 3H, J=7.3 Hz); 1.3 (m, 2H); 1.52 (m, 8H); 1.65 (m, 4H); 2.43 (t, 2H, J=7.6 Hz); 4.72 (s, 2H); 6.87 (m, 1H); 6.94 (m, 1H); 7.22 (t, 1H, J=9.4 Hz); 7.3 (d, 2H, J=8.5 Hz); 7.5 (d, 2H, J=7.3 Hz).

MS (ESI): 481 (M−H)

### Compound 70. 2-butyl-1-[(3′-((1-carboxy-1-methylmethyl)oxy)-3-methyl-biphenyl-4-yl)methyl]-4-spirocyclohexyl-1H-imidazol-5(4H)one

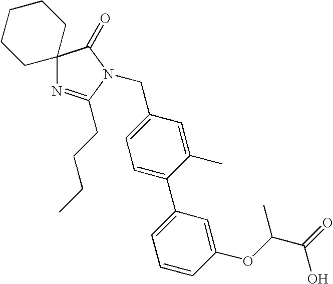

Prepared following the general procedure previously described (Method 15A) using 2-butyl-1-[(3′-((1-ethoxycarbonyl-1-methylmethyl)oxy)-3-methyl-biphenyl-4-yl)methyl]-4-spirocyclohexyl-1H-imidazol-5(4H)one (example 14.19). The product was chromatographed over silica gel (eluent dichloromethane/methanol 95/5). The product was obtained as a white solid.

Yield: 43.2%

Rf (dichloromethane/methanol 90/10): 0.4

MP: 81° C.

IR: νCO 1730 cm⁻¹

NMR ¹H (DMSO): 0.8 (t, 3H, J=7.3 Hz); 1.49 (m, 17H); 2.18 (s, 3H); 2.36 (t, 2H, J=7.6 Hz); 4.67 (s, 2H); 4.83 (q, 1H, J=6.7 Hz); 6.75 (m, 1H); 6.85 (m, 2H); 6.97 (m, 1H); 7.05 (s, 1H); 7.16 (d, 1H, J=7.9 Hz); 7.32 (t, 1H, J=7.9 Hz); 13.03 (s, 1H).

MS (ESI): 475 (M−H)

### Compound 71. 2-butyl-1-[(3′-((1-carboxy-1-ethylmethyl)oxy)-3-methyl-biphenyl-4-yl)methyl]-4-spirocyclohexyl-1H-imidazol-5(4H)one

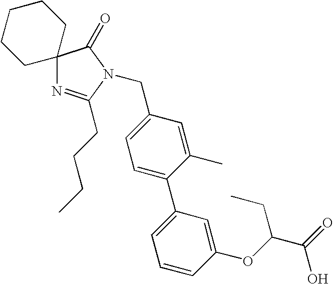

Prepared following the general procedure previously described (Method 15A) using 2-butyl-1-[(3′-((1-ethoxycarbonyl-1-ethylmethyl)oxy)-3-methyl-biphenyl-4-yl)methyl]-4-spirocyclohexyl-1H-imidazol-5(4H)one (example 14.20). The product was chromatographed over silica gel (eluent dichloromethane/methanol 95/5). The product was obtained as a white solid.

Yield: 73.2%

Rf (dichloromethane/methanol 90/10): 0.4

MP: 77° C.

IR: νCO 1731 cm⁻¹

NMR ¹H (DMSO): 0.8 (t, 3H, J=7 Hz); 0.99 (t, 3H, J=7.3 Hz); 1.41 (m, 14H); 1.88 (m, 2H); 2.19 (s, 3H); 2.36 (t, 2H, J=7.6 Hz); 4.67 (m, 3H); 6.76 (m, 1H); 6.86 (m, 2H); 6.97 (m, 1H); 7.05 (m, 1H); 7.16 (d, 1H, J=7.6 Hz); 7.32 (t, 1H, J=7.9 Hz); 13.01 (s, 1H).

MS (ESI): 489 (M−H)

### Compound 72. 2-butyl-1-[(3′-((1-carboxy-1,1-dimethylmethyl)oxy)-3-methyl-biphenyl-4-yl)methyl]-4-spirocyclohexyl-1H-imidazol-5(4H)one

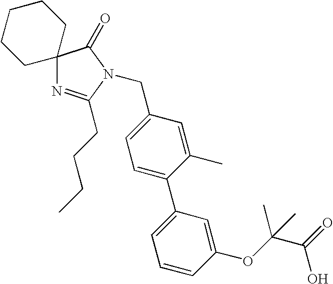

Prepared following the general procedure previously described (Method 15A) using 2-butyl-1-[(3′-((1-ethoxycarbonyl-1,1-dimethylmethyl)oxy)-3-methyl-biphenyl-4-yl)methyl]-4-spirocyclohexyl-1H-imidazol-5(4H)one (example 14.21). The product was chromatographed over silica gel (eluent dichloromethane/methanol 95/5). The product was obtained as a white solid.

Yield: 63.6%

Rf (dichloromethane/methanol 90/10): 0.4

MP: 140° C.

IR: νCO 1737 cm⁻¹

NMR ¹H (DMSO): 0.8 (t, 3H, J=7.3 Hz); 1.29 (m, 6H); 1.51 (m, 8H); 1.63 (m, 6H); 2.18 (s, 3H); 2.36 (t, 2H, J=7.6 Hz); 4.66 (s, 2H); 6.71 (m, 1H); 6.82 (m, 1H); 6.9 (m, 1H); 6.97 (m, 1H); 7.05 (m, 1H); 7.15 (m, 1H, J=7.9 Hz); 7.32 (t, 1H, J=7.9 Hz); 13.06 (s, 1H).

MS (ESI): 489 (M−H)

### Compound 73. 2-butyl-1-[(3′-((1-carboxy-1-(1,1-dimethylmethyl)methyl)oxy)-3-methyl-biphenyl-4-yl)methyl]-4-spirocyclohexyl-1H-imidazol-5(4H)one

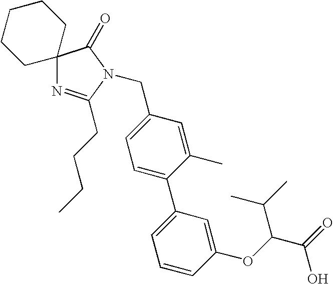

Prepared following the general procedure previously described (Method 15A) using 2-butyl-1-[(3′-((1-ethoxycarbonyl-1-(1,1-dimethylmethyl)methyl)oxy)-3-methyl-biphenyl-4-yl)methyl]-4-spirocyclohexyl-1H-imidazol-5(4H)one (example 14.22). The product was chromatographed over silica gel (eluent dichloromethane/methanol 95/5). The product was obtained as a white solid.

Yield: 84.4%

Rf (dichloromethane/methanol 90/10): 0.4

MP: 92° C.

IR: νCO 1731 cm⁻¹

NMR ¹H (DMSO): 0.8 (t, 3H, J=7.3 Hz); 1.01 (d, 6H, J=6.7 Hz); 1.27 (m, 6H); 1.49 (m, 2H); 1.67 (m, 6H); 2.19 (m, 4H); 2.36 (t, 2H, J=7.62); 4.48 (d, 1H, J=5 Hz); 4.67 (s, 2H); 6.76 (m, 1H); 6.86 (m, 2H); 6.97 (m, 1H, J=7.6 Hz); 7.05 (s, 1H); 7.16 (d, 1H, J=7.9 Hz); 7.32 (t, 1H, J=7.9 Hz); 12.99 (s, 1H).

MS (ESI): 503 (M−H)

### Compound 74. 2-butyl-1-[(3′-((1-carboxy-1,1-dimethylmethyl)oxy)biphenyl-3-yl)methyl]-4-spirocyclopentyl-1H-imidazol-5(4H)-one

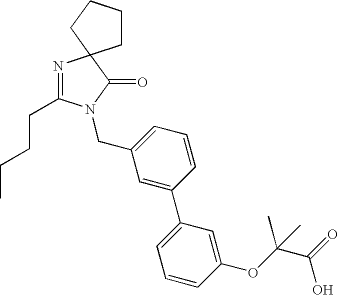

Prepared following the general procedure previously described (Method 15A) using 2-butyl-1-[(3′-((1-ethoxycarbonyl-1,1-dimethylmethyl)oxy)biphenyl-3-yl)methyl]-4-spirocyclopentyl-1H-imidazol-5(4H)-one (example 14.23). The product was chromatographed over silica gel (eluent dichloromethane/methanol 95/5). The product was obtained as a white solid.

Yield: 25%

Rf (dichloromethane/methanol 90/10): 0.4

MP: 70° C.

IR: νCO 1.731 cm⁻¹

NMR ¹H (DMSO): 0.77 (t, 3H, J=7.3 Hz); 1.24 (m, 3H); 1.48 (m, 2H); 1.54 (s, 6H); 1.75 (m, 1H); 1.86 (s, 6H); 2.43 (m, 2H); 4.80 (s, 2H); 6.82 (dd, 1H, J=2 Hz J=7.9 Hz); 7.05 (d, 1H, J=2 Hz); 7.14 (m, 1H); 7.20 (m, 1H); 7.37 (t, 1H, J=7.9 Hz); 7.45 (m, 2H); 7.51 (m, 1H).

MS (ESI): 463 (M+H)

### Compound 75. 2-butyl-1-[(2′-((1-carboxy-1,1-dimethylmethyl)oxy)biphenyl-3-yl)methyl]-4-spirocyclopentyl-1H-imidazol-5(4H)-one

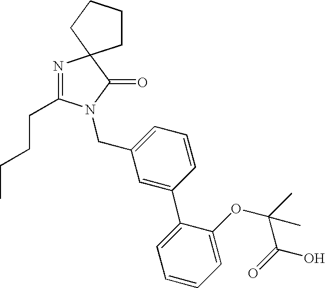

Prepared following the general procedure previously described (Method 15A) using 2-butyl-1-[(2′-((1-ethoxycarbonyl-1,1-dimethylmethyl)oxy)biphenyl-3-yl)methyl]-4-spirocyclopentyl-1H-imidazol-5(4H)-one (example 14.24). The product was chromatographed over silica gel (eluent dichloromethane/methanol 95/5). The product was obtained as a white solid.

Yield: 25%

Rf (dichloromethane/methanol 90/10): 0.4

MP: 167° C.

IR: νCO 1742 cm⁻¹

NMR ¹H (DMSO): 0.77 (t, 3H, J=7.3 Hz); 1.23 (m, 3H); 1.37 (s, 6H); 1.47 (m, 2H); 1.69 (m, 1H); 1.84 (s, 6H); 2.38 (m, 2H); 4.75 (s, 2H); 6.82 (d, 1H, J=8.5 Hz); 7.05 (t, 1H, J=7.3 Hz); 7.13 (m, 1H); 7.27 (m, 3H); 7.40 (d, 2H, J=4.7 Hz).

MS (ESI): 463 (M+H)

### Compound 76. 2-butyl-1-[(3′-((1-carboxy-1,1-dimethylmethyl)oxy)-6′-propyl-biphenyl-4-yl)methyl]-4-spirocyclopentyl-1H-imidazol-5(4H)-one

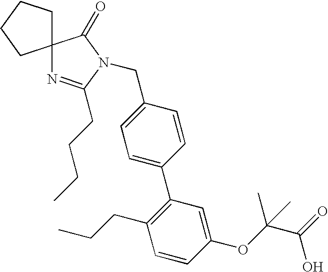

Prepared following the general procedure previously described (Method 15A) using 2-butyl-1-[(3′-((1-ethoxycarbonyl-1,1-dimethylmethyl)oxy)-6′-propyl-biphenyl-4-yl)methyl]-4-spirocyclopentyl-1H-imidazol-5(4H)-one (example 14.25). The product was chromatographed over silica gel (eluent dichloromethane/methanol 92/8). The product was obtained as a white solid.

Yield: 89%

Rf (dichloromethane/methanol 90/10): 0.4

MP <60° C.

IR: νCO 1721 cm⁻¹

NMR ¹H (CDCl₃): 0.80 (m, 6H); 1.27 (m, 2H); 1.42 (m, 2H); 1.52 (m, 2H); 1.59 (s, 6H); 1.88 (m, 8H); 2.41 (m, 4H); 4.73 (s, 2H); 6.75 (d, 1H, J=2.6 Hz); 6.87 (dd, 1H, J=8.4 Hz, J=2.6 Hz); 7.15 (m, 3H); 7.23 (d, 2H, J=8.2 Hz).

MS (ESI): 505 (M+H)

### Compound 77. 2-butyl-1-[(4′-((1-carboxy-1,1-dimethylmethyl)oxy)biphenyl-3-yl)methyl]-4-spirocyclopentyl-1H-imidazol-5(4H)-one

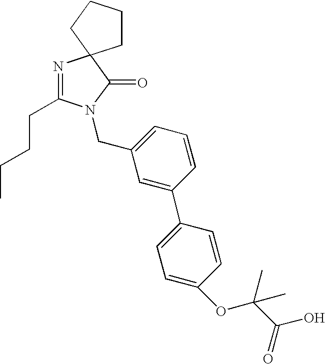

Prepared following the general procedure previously described (Method 15A) using 2-butyl-1-[(4′-((1-ethoxycarbonyl-1,1-dimethylmethyl)oxy)biphenyl-3-yl)methyl]-4-spirocyclopentyl-1H-imidazol-5(4H)-one (example 14.26). The product was chromatographed over silica gel (eluent dichloromethane/methanol 92/8). The product was obtained as a white solid.

Yield: 84%

Rf (dichloromethane/methanol 90/10): 0.3

MP: 170° C.

IR: νCO 1727 cm⁻¹

NMR ¹H (DMSO): 0.74 (t, 3H, J=7.3 Hz); 1.20 (m, 2H); 1.47 (m, 2H); 1.64 (s, 6H); 1.94 (m, 8H); 2.37 (m, 2H); 4.72 (s, 2H); 6.95 (d, 2H, J=8.6 Hz); 7.07 (d, 1H, J=7.4 Hz); 7.09 (d, 1H, J=7.6 Hz); 7.37 (m, 4H); 10.26 (s, 1H).

MS (ESI): 463 (M+H)

### Compound 78. 2-butyl-1-[(3′-((1-carboxy-1,1-dimethylmethyl)oxy)-2′-fluoro-biphenyl-4-yl)methyl]-4-spirocyclopentyl-1H-imidazol-5(4H)-one

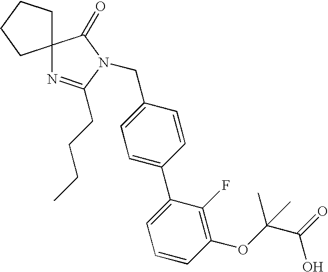

Prepared following the general procedure previously described (Method 15A) using 2-butyl-1-[(3′-((1-ethoxycarbonyl-1,1-dimethylmethyl)oxy)-2′-fluoro-biphenyl-4-yl)methyl]-4-spirocyclopentyl-1H-imidazol-5(4H)-one (example 14.27). The product was chromatographed over silica gel (eluent dichloromethane/methanol 92/8). The product was obtained as a white solid.

Yield: 77%

Rf (dichloromethane/methanol 90/10): 0.4

MP: 162° C.

IR: νCO 1732 cm⁻¹

NMR ¹H (DMSO): 0.79 (t, 3H, J=6.3 Hz); 1.27 (m, 2H); 1.43-1.52 (m, 8H); 1.67 (m, 2H); 1.83 (m, 6H); 2.35 (t, 2H, J=7.9 Hz); 4.73 (s, 2H); 6.97 (m, 1H); 7.08-7.19 (m, 2H); 7.24 (d, 2H, J=7.1 Hz); 7.51 (d, 2H, J=7.1 Hz); 13.16 (s, 1H).

MS (ESI): 481 (M+H)

### Compound 79. 2-butyl-1-[(3′-((1-carboxy-1,1-dimethylmethyl)oxy)-4′-methoxy-biphenyl-4-yl)methyl]-4-spirocyclopentyl-1H-imidazol-5(4H)-one

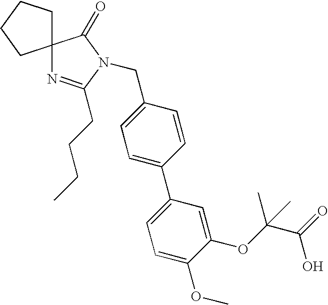

Prepared following the general procedure previously described (Method 15A) using 2-butyl-1-[(3′-((1-ethoxycarbonyl-1,1-dimethylmethyl)oxy)-4′-methoxy-biphenyl-4-yl)methyl]-4-spirocyclopentyl-1H-imidazol-5(4H)-one (example 14.28). The product was chromatographed over silica gel (eluent dichloromethane/methanol 92/8). The product was obtained as a viscous oil.

Yield: 78.9%

Rf (dichloromethane/methanol 90/10): 0.6

IR: νCO 1729 cm⁻¹

NMR ¹H (CDCl₃): 1.22-1.37 (m, 2H); 1.50-1.60 (m, 2H); 1.61 (s, 6H); 1.83-2.03 (m, 8H); 2.35-2.41 (m, 2H); 3.91 (s, 3H); 4.72 (s, 2H); 6.99 (d, 1H, J=8.4 Hz); 7.19 (d, 2H, J=8.1 Hz); 7.25-7.30 (m, 2H); 7.49 (d, 2H, J=8.1 Hz).

MS (ESI): 493 (M+H)

### Compound 80. 2-butyl-1-[(3′-((1-carboxy-1,1-dimethylmethyl)oxy)-6′-ethyl-biphenyl-4-yl)methyl]-4-spirocyclopentyl-1H-imidazol-5(4H)-one

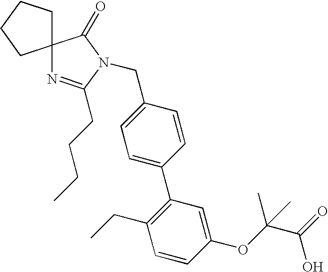

Prepared following the general procedure previously described (Method 15A) using 2-butyl-1-[(3′-((1-ethoxycarbonyl-1,1-dimethylmethyl)oxy)-6′-ethyl-biphenyl-4-yl)methyl]-4-spirocyclopentyl-1H-imidazol-5(4H)-one (example 14.29). The product was chromatographed over silica gel (eluent dichloromethane/methanol 95/5). The product was obtained as a white solid.

Yield: 82%

Rf (dichloromethane/methanol 90/10): 0.3

MP: 143° C.

IR: νCO 1626 and 1724 cm⁻¹

NMR ¹H (CDCl₃): 0.81 (t, 3H, J=7.3 Hz); 1.00 (t, 3H, J=7.1 Hz); 1.23-1.32 (m, 2H); 1.43-1.57 (m, 8H); 1.84-2.01 (m, 8H); 2.36-2.40 (m, 2H); 2.44 (q, 2H, J=7.1 Hz); 4.71 (s, 2H); 6.74 (d, 1H); 6.84 (dd, 1H); 7.06 (d, 1H, J=7.5 Hz); 7.13 (d, 2H, J=7.7 Hz); 7.21 (d, 2H, J=7.4 Hz).

MS (ESI): 491 (M+H)

### Compound 81. 2-butyl-1-[(3′-((1-carboxy-1,1-dimethylmethyl)oxy)-4′-isobutyl-biphenyl-4-yl)methyl]-4-spirocyclopentyl-1H-imidazol-5(4H)-one

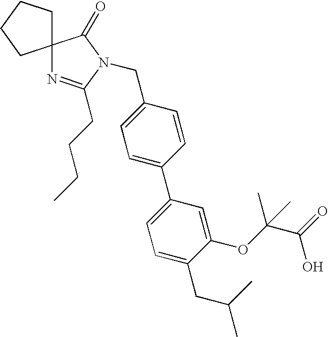

Prepared following the general procedure previously described (Method 15A) using 2-butyl-1-[(3′-((1-ethoxycarbonyl-1,1-dimethylmethyl)oxy)-4′-isobutyl-biphenyl-4-yl)methyl]-4-spirocyclopentyl-1H-imidazol-5(4H)-one (example 14.30). The product was chromatographed over silica gel (eluent dichloromethane/methanol 95/5). The product was obtained as a white solid.

Yield: 79%

Rf (dichloromethane/methanol 90/10): 0.45

MP: 78° C.

IR: νCO 1624 and 1736 cm⁻¹

NMR ¹H (CDCl₃): 0.71 (t, 3H, J=7.2 Hz); 0.92 (d, 6H, J=6.5 Hz); 1.28-1.31 (m, 2H); 1.53-1.60 (m, 3H); 1.65 (s, 6H); 1.84-2.00 (m, 8H); 2.35-2.41 (m, 2H); 2.51 (d, 2H, J=6.9 Hz); 4.67 (s, 2H); 7.00-7.14 (m, 5H); 7.46 (d, 2H, J=7.8 Hz).

MS (ESI): 519 (M+H)

### Compound 82. 2-butyl-1-[(3′-((1-carboxy-1,1-dimethylmethyl)oxy)-2-methoxy-biphenyl-4-yl)methyl]-4-spirocyclopentyl-1H-imidazol-5(4H)-one

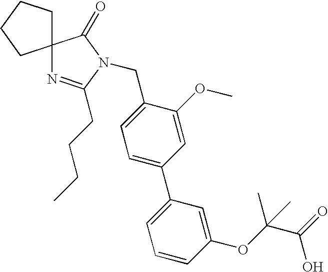

Prepared following the general procedure previously described (Method 15A) using 2-butyl-1-[(3′-((1-ethoxycarbonyl-1,1-dimethylmethyl)oxy)-2-methoxy-biphenyl-4-yl)methyl]-4-spirocyclopentyl-1H-imidazol-5(4H)-one (example 14.31). The product was chromatographed over silica gel (eluent dichloromethane/methanol 95/5). The product was obtained as a white solid.

Yield: 75%

Rf (dichloromethane/methanol 90/10): 0.4

MP: 154° C.

IR: νCO 1727 cm⁻¹

NMR ¹H (CDCl₃): 0.82 (t, 3H, J=7.3 Hz); 1.20-1.30 (m, 2H); 1.45-1.55 (m,: 2H); 1.60 (s, 6H); 1.82-1.98 (m, 8H); 1.36-1.40 (m, 2H); 3.88 (s, 3H); 4.73 (s, 2H); 6.90 (d, 1H, J=6.2 Hz); 6.99-7.10 (m, 3H); 7.16-7.20 (m, 2H); 7.28 (s, 1H); 8.82 (s, 1H).

MS (ESI): 493 (M+H)

### Compound 83. 2-butyl-1-[(3′-((1-carboxy-1-methylmethyl)oxy)-6′-propyl-biphenyl-4-yl)methyl]-4-spirocyclopentyl-1H-imidazol-5(4H)-one

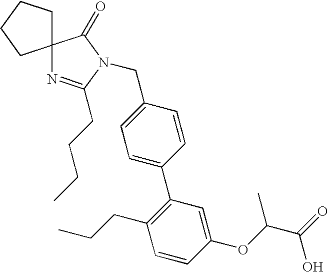

Prepared following the general procedure previously described (Method 15A) using 2-butyl-1-[(3′-((1-ethoxycarbonyl-1-methylmethyl)oxy)-6′-propyl-biphenyl-4-yl)methyl]-4-spirocyclopentyl-1H-imidazol-5(4H)-one (example 14.32). The product was chromatographed over silica gel (eluent dichloromethane/methanol 90/10). The product was obtained as a white solid.

Yield: 23.8%

Rf (dichloromethane/methanol 90/10): 0.2

MP: 76° C.

IR: νCO 1730 cm⁻¹

NMR ¹H (DMSO): 0.69 (t, 3H, J=7 Hz); 0.76 (t, 3H, J=7.3 Hz); 1.17-1.50 (m, 9H); 1.68 (m, 2H); 1.84 (m, 6H); 2.35 (m, 4H); 4.75 (m, 3H); 6.56 (d, 1H, J=2.7 Hz); 6.78 (dd, 1H, J=8.5 Hz, J=2.7 Hz); 7.17 (m, 3H); 7.25 (d, 2H, J=8.2 Hz).

MS (ESI): 489 (M−H)

### Compound 84. 2-butyl-1-[(3′-((1-carboxymethyl)oxy)-6′-propyl-biphenyl-4-yl)methyl]-4-spirocyclopentyl-1H-imidazol-5(4H)-one

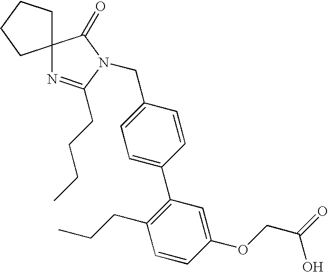

Prepared following the general procedure previously described (Method 15A) using 2-butyl-1-[(3′-((1-ethoxycarbonyl-methyl)oxy)-6′-propyl-biphenyl-4-yl)methyl]-4-spirocyclopentyl-1H-imidazol-5(4H)-one (example 14.33). The product was chromatographed over silica gel (eluent dichloromethane/methanol 90/10). The product was obtained as a white solid.

Yield: 42.4%

Rf (dichloromethane/methanol 90/10): 0.2

MP: 76° C.

IR: νCO 1729 cm⁻¹

NMR ¹H (DMSO): 0.69 (t, 3H, J=7.3 Hz); 0.76 (t, 3H, J=7.3 Hz); 1.18-1.50 (m, 6H); 1.67 (m, 2H); 1.84 (m, 6H); 2.36 (m, 4H); 4.54 (s, 2H); 4.73 (s, 2H); 6.60 (d, 1H, J=2.9 Hz); 6.81 (dd, 1H, J=8.5 Hz, J=2.9 Hz); 7.17 (m, 3H); 7.26 (d, 2H, J=8.2 Hz).

MS (ESI): 475 (M−H)

### Compound 85. 2-butyl-1-[(3′-((1-carboxy-1-ethylmethyl)oxy)-6′-propyl-biphenyl-4-yl)methyl]-4-spirocyclopentyl-1H-imidazol-5(4H)-one

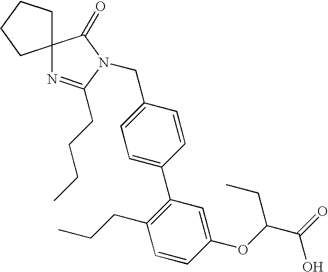

Prepared following the general procedure previously described (Method 15A) using 2-butyl-1-[(3′-((1-ethoxycarbonyl-1-ethylmethyl)oxy)-6′-propyl-biphenyl-4-yl)methyl]-4-spirocyclopentyl-1H-imidazol-5(4H)-one (example 14.34). The product was chromatographed over silica gel (eluent dichloromethane/methanol 90/10). The product was obtained as a white solid.

Yield: 52.9%

Rf (dichloromethane/methanol 90/10): 0.2

MP: 74° C.

IR: νCO 1738 and 1775 cm⁻¹

NMR ¹H (DMSO): 0.70 (t, 3H, J=7.3 Hz); 0.79 (t, 3H, J=7.3 Hz); 0.98 (t, 3H, J=7.3 Hz); 1.27 (m, 4H); 1.46 (m, 2H); 1.87 (m, 10H); 2.41 (t, 2H, J=7.9 Hz); 2.54 (m, 2H); 4.60 (t, 1H, J=5.3 Hz); 4.84 (s, 2H); 6.60 (d, 1H, J=2.6 Hz); 6.81 (dd, 1H, J=8.5 Hz, J=2.6 Hz); 7.18 (d, 1H, J=8.5 Hz); 7.28 (m, 4H).

MS (ESI): 503 (M−H)

### Compound 86. 2-butyl-1-[(3′-((1-carboxy-1-(1,1-dimethylmethyl)methyl)oxy)-6′-propyl-biphenyl-4-yl)methyl]-4-spirocyclopentyl-1H-imidazol-5(4H)-one

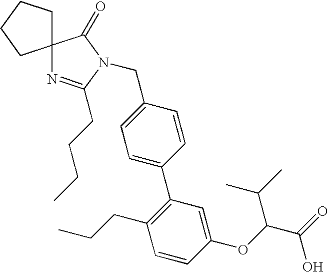

Prepared following the general procedure previously described (Method 15A) using 2-butyl-1-[(3′-((1-ethoxycarbonyl-1-(1,1-dimethylmethyl)methyl)oxy)-6′-propyl-biphenyl-4-yl)methyl]-4-spirocyclopentyl-1H-imidazol-5(4H)-one (example 14.35). The product was chromatographed over silica gel (eluent dichloromethane/methanol 90/10). The product was obtained as a white solid.

Yield: 69.3%

Rf (dichloromethane/methanol 90/10): 0.2

MP: 81° C.

IR: vCO 1736 and 1775 cm⁻¹

NMR ¹H (DMSO): 0.70 (t, 3H, J=7.3 Hz); 0.79 (t, 3H, J=7.3 Hz); 0.99 (d, 6H, J=6.8 Hz); 1.33 (m, 4H); 1.48 (m, 2H); 1.87 (m, 8H); 2.16 (m, 1H); 2.40 (t, 2H, J=8.2 Hz); 2.51 (m, 2H); 4.41 (d, 1H, J=5.3 Hz); 4.83 (s, 2H); 6.60 (d, 1H, J=2.6 Hz); 6.80 (dd, 1H, J=8.5 Hz, J=2.6 Hz); 7.19 (d, 1H, J=8.5 Hz); 7.28 (m, 4H); 12.93 (s, 1H).

MS (ESI): 519 (M+H)

### Compound 87. 2-butyl-1-[(3∝-((1-carboxy-1,1-dimethylmethyl)oxy)-6′-isobutyl-biphenyl-4-yl)methyl]-4-spirocyclopentyl-1H-imidazol-5(4H )-one

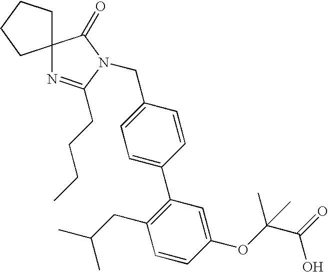

Prepared following the general procedure previously described (Method 15A) using 2-butyl-1-[(3′-((1-ethoxycarbonyl-1,1-dimethylmethyl)oxy)-6′-isobutyl-biphenyl-4-yl)methyl]-4-spirocyclopentyl-1H-imidazol-5(4H)-one (example 14.36).

The product was chromatographed over silica gel (eluent dichloromethane/methanol 95/5). The product was obtained as a white solid.

Yield: 90%

Rf (dichloromethane/methanol 90/10): 0.3

MP<50° C.

IR: vCO 1731 c⁻¹

NMR ¹H (DMSO): 0.68 (d, 6H, J=6.6 Hz); 0.81 (t, 3H, J=7.2 Hz); 1.20-1.35 (m, 2H); 1.48-1.60 (m, 3H); 1.60 (s, 6H); 1.85-2.03 (m, 8H); 2.36-2.44 (m, 4H); 4.74 (s, 2H); 6.76 (d, 1H, J=2.5 Hz); 6.84 (dd, 1H, J=8.4 Hz, J=2.5 Hz); 7.07 (d, 1H, J=8.4 Hz); 7.13 (d, 2H, J=8.1 Hz); 7.23 (d, 2H, J=8.1 Hz); 12.30 (s, 1H).

MS (ESI): 519 (M+H)

### Compound 88. 2-butyl-1-[(3′-((1-carboxy-1,1-dimethylmethyl)oxy)-3-ethyl-biphenyl-4-yl)methyl]-4-spirocyclopentyl-1H-imidazol-5(4H)-one

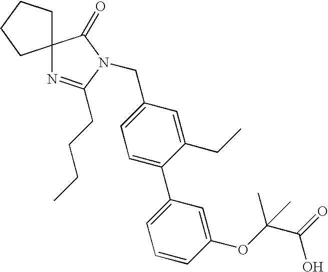

Prepared following the general procedure previously described (Method 15A) using 2-butyl-1-[(3′-((1-ethoxycarbonyl-1,1-dimethylmethyl)oxy)-3-ethyl-biphenyl-4-yl)methyl]-4-spirocyclopentyl-1H-imidazol-5(4H)-one (example 14.37). The product was chromatographed over silica gel (eluent dichloromethane/methanol 95/5). The product was obtained as a white solid.

Yield: 83%

Rf (dichloromethane/methanol 90/10): 0.45

MP: 160° C.

IR: vCO 1732 cm⁻¹

NMR ¹H (DMSO): 0.80 (t, 3H, J=7.5 Hz); 1.01 (t, 3H, J=7.5 Hz); 1.23-1.32 (m, 2H); 1.44-1.55 (m, 8H); 1.67-1.86 (m, 8H); 2.34-2.40 (m, 2H); 2.52 (q, 2H, J=7.5 Hz); 4.71 (s, 2H); 6.71 (s, 1H); 6.84-6.87 (m, 2H); 7.01 (d, 1H, J=7.5 Hz); 7.10-7.15 (m, 2H); 7.29 (m, 1H); 13.34 (s, 1H).

MS (ESI): 491 (M+H)

### Compound 89. 2-butyl-1-[(3′-((1-carboxy-1.1-dimethylmethyl)oxy)-6′-cyano-biphenyl-4-yl)methyl]-4-spirocyclopentyl-1H-imidazol-5(4H)-one

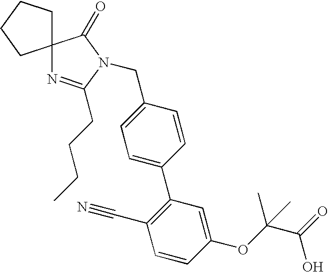

Prepared following the general procedure previously described (Method 15A) using 2-butyl-1-[(3′-((1-ethoxycarbonyl-1,1-dimethylmethyl)oxy)-6′-cyano-biphenyl-4-yl)methyl]-4-spirocyclopentyl-1H-imidazol-5(4H)-one (example 14.38). The product was chromatographed over silica gel (eluent dichloromethane/methanol 95/5). The product was obtained as a white solid.

Yield: 83%

Rf (dichloromethane/methanol 90/10): 0.15

MP<50° C.

IR: vCO 1735 cm⁻¹

NMR ¹H (DMSO): 0.79 (t, 3H, J=7.2Hz); 1.17-1.32 (m, 2H); 1.47-1.57 (m, 2H); 1.73 (s, 6H); 1.87-2.04 (m, 8H); 2.39-2.46 (m, 2H); 4.76 (s, 2H); 6.93 (d, 1H, J=2.4 Hz); 7.00 (dd, 1H, J=8.6 Hz, J=2.4Hz); 7.25 (d, 2H, J=8.2 Hz); 7.51 (d, 2H, J=8.2 Hz); 7.66 (d,1H, J=8.6 Hz).

MS (ESI): 488 (M+H)

### Compound 90. 2-butyl-1-[(3′-((1-carboxy-1,1-dimethylmethyl)oxy)-3-methoxy-biphenyl-4-yl )methyl1-4-spirocyclopentyl-1H-imidazol-5(4H )-one

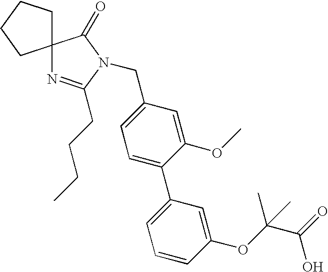

Prepared following the general procedure previously described (Method 15A) using 2-butyl-1-[(3′-((1-ethoxycarbonyl-1,1-dimethylmethyl)oxy)-3-methoxy-biphenyl-4-yl)methyl]-4-spirocyclopentyl-1H-imidazol-5(4H)-one (example 14.39).

The product was chromatographed over silica gel (eluent dichloromethane/methanol 95/5). The product was obtained as a white solid.

Yield: 85%

Rf (dichloromethane/methanol 90/10): 0.42

MP: 157° C.

IR: vCO 1728 cm⁻¹

NMR ¹H (DMSO): 0.73 (t, 3H, J=7.5 Hz); 1.10-1.19 (m, 2H); 1.28-1.37 (m, 2H); 1.53 (s, 6H); 1.62-1.82 (m, 8H); 1.99-2.05 (m, 2H); 3.72 (s, 3H); 4.59 (s, 2H); 6.49 (s, 1H); 6.78 (s, 1H); 6.86-6.96 (m, 3H); 7.21 (d, 1H, J=7.5 Hz); 7.33 (m, 1H); 13.18 (s, 1H).

MS (ESI): 493 (M+H)

### Compound 91. 2-butyl-1-[(3′-((1-carboxy-1,1-dimethylmethyl)oxy)-2-methyl-biphenyl-4-yl)methyl]-4-spirocyclopentyl-1H-imidazol-5(4H )-one

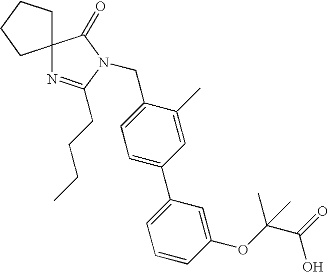

Prepared following the general procedure previously described (Method 15A) using 2-butyl-1-[(3′-((1-ethoxycarbonyl-1,1-dimethylmethyl)oxy)-2-methyl-biphenyl-4-yl)methyl]-4-spirocyclopentyl-1H-imidazol-5(4H)-one (example 14.40). The product was chromatographed over silica gel (eluent dichloromethane/methanol 95/5). The product was obtained as a white solid.

Yield: 83%

Rf (dichloromethane/methanol 90/10): 0.54

MP: 160° C.

IR: vCO 1732 cm⁻¹

NMR ¹H (DMSO): 0.78 (t, 3H, J=7.5 Hz); 1.19-1.33 (m, 2H); 1.43-1.55 (m, 8H); 1.72-1.88 (m, 8H); 2.28-2.36 (m, 5H); 4.71 (s, 2H); 6.85 (m, 2H); 7.09 (s, 1H); 7.23-7.48 (m, 4H); 13.21 (s,1H).

MS (ESI): 477 (M+H)

### Compound 92. 2-butyl-1-[[2-[(4-(1-carboxy-1,1-dimethylmethyloxy)phenyl]-6-methyl-thiazolo[3.2-b][1,2,4]triazol-5-yl]methyl]-4-spirocyclopentyl-1H-imidazol-5(4H)-one

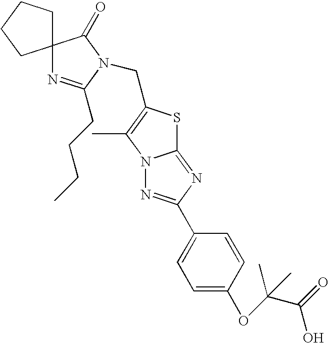

Prepared following the general procedure previously described (Method 15A) using 2-butyl-1-[[2-[(4-(1-ethoxycarbonyl-1,1-di methylmethyloxy)phenyl]-6-methyl-thiazolo[3,2-b][1,2,4]triazol-5-yl]methyl]-4-spirocyclopentyl-1H-imidazol-5(4H)-one (example 14.41). The product was chromatographed over silica gel (eluent dichloromethane/methanol 95/5). The product was obtained as a white solid.

Yield: 49.9%

Rf (dichloromethane/methanol 90/10): 0.4

MP: 110° C.

IR: vCO 1728 cm⁻¹

NMR ¹H (DMSO): 0.85 (t, 3H, J=7.3 Hz); 1.38 (m, 2H); 1.52-1.67 (m, 10H); 1.83 (m, 6H); 2.52 (m, 2H); 2.58 (s, 3H); 4.93 (s, 2H); 6.91 (d, 2H, J=9 Hz); 7.95 (d, 2H, J=9 Hz); 13.13 (s, 1H).

### Compound 93. 2-butyl-1-[[2-[(3-(1-carboxy-1,1-dimethylmethyloxy)phenyl]-6-methyl-thiazolo[3,2-b][1,2,4]triazol-5-yl]methyl]-4-spirocyclopentyl-1H-imidazol-5(4H)-one

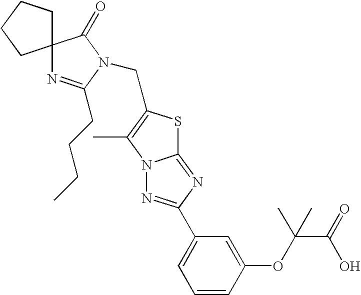

Prepared following the general procedure previously described (Method 15A) using 2-butyl-1-[[2-[(3-(1-ethoxycarbonyl-1,1-dimethylmethyloxy)phenyl]-6-methyl-thiazolo[3,2-b][1,2,4]triazol-5-yl]methyl]-4-spirocyclopentyl-1H-imidazol-5(4H)-one (example 14.42). The product was chromatographed over silica gel (eluent dichloromethane/methanol 95/5). The product was obtained as a white solid.

Yield: 61.7%

Rf (dichloromethane/methanol 90/10): 0.3

MP: 145° C.

IR: vCO 1725 cm⁻¹

NMR ¹H (DMSO): 0.84 (t, 3H, J=7.3 Hz); 1.32 (m, 2H); 1.56 (m, 10H); 1.81 (m, 6H); 2.50 (m, 2H); 2.58 (s, 3H); 4.92 (s, 2H); 6.94 (m, 1H); 7.33 (m, 1H); 7.55 (m, 1H); 7.63 (d,1H, J=7.6 Hz).

### Compound 94. 2-butyl-1-[(3′-((1-carboxy-1,1-dimethylmethyl)oxy)-3-propyl-biphenyl-4-yl)methyl]-4-spirocyclopentyl-1H-imidazol-5(4H)-one

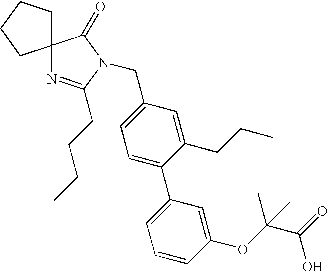

Prepared following the general procedure previously described (Method 15A) using 2-butyl-1-[(3′-((1-ethoxycarbonyl-1,1-dimethylmethyl)oxy)-3-propyl-biphenyl-4-yl)methyl]-4-spirocyclopentyl-1H-imidazol-5(4H)-one (example 14.43). The product was chromatographed over silica gel (eluent dichloromethane/methanol 98/2). The product was obtained as a white solid.

Yield: 71%

Rf (dichloromethane/methanol 90/10): 0.5

MP: 59° C.

IR: vCO 1731 cm⁻¹

NMR ¹H (DMSO): 0.76-0.86 (m, 6H); 1.22-1.63 (m, 12H); 1.92-2.04 (m, 8H); 2.42-2.56 (m, 4H); 4.72 (s, 2H); 6.87-7.04 (m, 4H); 7.04 (s, 1H); 7.14 (d, 1H, J=7.5 Hz); 7.27 (m, 1H); 8.68 (s, 1H).

MS (ESI): 503 (M+H)

### Compound 95. 2-butyl-1-[(3′-((1-carboxy-1,1-dimethylmethyl)oxy)-4′-nitro-biphenyl-4-yl)methyl]-4-spirocyclopentyl-1H-imidazol-5(4H)-one

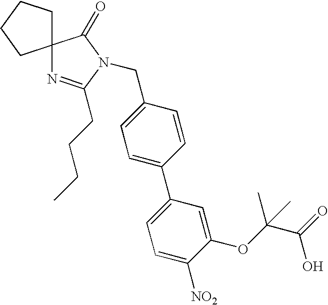

Prepared following the general procedure previously described (Method 15A) using 2-butyl-1-[(3′-((1-ethoxycarbonyl-1,1-dimethylmethyl)oxy)-4′-nitro-biphenyl-4-yl)methyl]-4-spirocyclopentyl-1H-imidazol-5(4H)-one (example 14.44). The product was chromatographed over silica gel (eluent dichloromethane/methanol 98/2) yellow solid.

Yield: 77%

Rf (dichloromethane/methanol 90/10): 0.4

MP<50° C.

IR: vCO 1734 cm⁻¹

NMR ¹H (DMSO): 0.74 (t, 3H, J=7.2 Hz); 1.15-1.29 (m, 2H); 1.45-1.57 (m, 2H); 1.71 (s, 6H); 1.84-1.99 (m, 8H); 2.40-2.46 (m, 2H); 4.73 (s, 2H); 7.19-7.22 (m, 3H); 7.29 (s, 1H); 7.49 (d, 2H, J=8.1 Hz); 7.85 (d, 1H, J=8.4Hz); 10.11 (s, 1H).

MS (ESI): 508 (M+H)

### Compound 96. 2-butyl-1-[(3′-((1-carboxy-1.1-dimethylmethyl)oxy)-3-trifluoromethyl-biphenyl-4-yl)methyl1-4-spirocyclopentyl-1H-imidazol-5(4H)-one

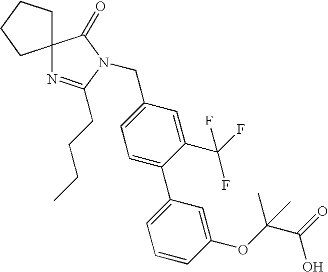

Prepared following the general procedure previously described (Method 15A) using 2-butyl-1-[(3′-((1-ethoxycarbonyl-1,1-dimethylmethyl)oxy)-3-trifluoromethyl-biphenyl-4-yl)methyl]-4-spirocyclopentyl-1H-imidazol-5(4H )-one (example 14.45). The product was chromatographed over silica gel (eluent dichloromethane/methanol 98/2). The product was obtained as a white solid.

Yield: 80%

Rf (dichloromethane/methanol 90/10): 0.54

MP: 90° C.

IR: vCO 1735 cm⁻¹

NMR ¹H (DMSO): 0.77 (t, 3H, J=8 Hz); 1.21-1.30 (m, 2H); 1.42-1.49 (m, 8H); 1.66-1.84 (m, 8H); 2.36-2.39 (m, 2H); 4.83 (s, 2H); 6.73 (s, 1H); 6.87-6.89 (m, 2H); 7.31 (m, 1H); 7.37-7.45 (m, 2H); 7.59 (s, 1H); 13.15 (s, 1H).

MS (ESI): 531 (M+H)

### Compound 97. 2-butyl-1-[(3′-((1-carboxy-1,1-dimethylmethyl)oxy)-3-nitro-biphenyl-4-yl)methyl]-4-spirocyclopentyl-1H-imidazol-5(4H )-one

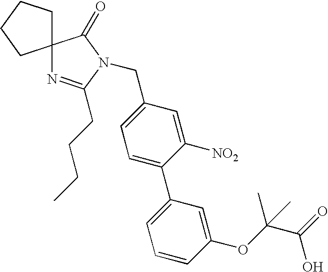

Prepared following the general procedure previously described (Method 15A) using 2-butyl-1-[(3′-((1-ethoxycarbonyl-1,1-dimethylmethyl)oxy)-3-nitro-biphenyl-4-yl)methyl]-4-spirocyclopentyl-1H-imidazol-5(4H)-one (example 14.46). The product was chromatographed over silica gel (eluent dichloromethane/methanol 90/10).

The product was obtained as a yellow solid.

Yield: 82%

Rf (dichloromethane/methanol 90/10): 0.54

MP: 101° C.

IR: vCO 1731 cm⁻¹

NMR ¹H (DMSO): 0.82 (t, 3H, J=6.8 Hz); 1.26-1.33 (m, 2H); 1.52 (m, 8H); 1.69-1.86 (m, 8H); 2.39-2.44 (m, 2H); 4.85 (s, 2H); 6.75 (s, 1H); 7.88-7.98 (m, 2H); 7.35 (m,1H); 7.52-7.58 (m, 2H); 7.80 (s, 1H); 13.25 (s, 1H).

MS (ESI): 508 (M+H)

### Compound 98. 2-butyl-1-[(3′-((1-carboxy-1,1-dimethylmethyl)oxy)-4′-propyl-biphenyl-4-yl)methyl]-4-spirocyclopentyl-1H-imidazol-5(4H)-one

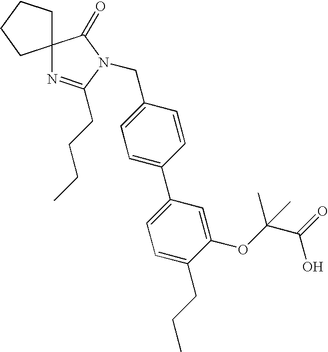

Prepared following the general procedure previously described (Method 15A) using 2-butyl-1-[(3′-((1-ethoxycarbonyl-1,1-dimethylmethyl)oxy)-4′-propyl-biphenyl-4-yl)methyl]-4-spirocyclopentyl-1H-imidazol-5(4H)-one (example 14.47). The product was chromatographed over silica gel (eluent dichloromethane/methanol 95/5). The product was obtained as a white solid.

Yield: 78%

Rf (dichloromethane/methanol 90/10): 0.8

MP: 74° C.

IR: vCO 1731 cm⁻¹

NMR ¹H (DMSO): 0.72 (t, 3H, J=7.3 Hz); 0.98 (t, 3H, J=7.3 Hz); 1.11-1.26 (m, 2H); 1.42-1.54 (m, 2H); 1.54-1.70 (m, 2H); 1.66 (s, 6H); 1.84-2.98 (m, 8H); 2.36-2.42 (m, 2H); 2.60-2.65 (m, 2H); 4.68 (s, 2H); 7.02 (d, 1H); 7.07-7.20 (m, 4H); 7.43 (d, 2H, J=8.2 Hz).

MS (ESI): 505 (M+H)

### Compound 99. 2-butyl-1-[(3′-((1-(tetrazol-5-yl)-1-ethylmethyl)oxy)-biophenyl-4-yl)methyl]-4-spirocyclopentyl-1H-imidazol-5(4H)-one

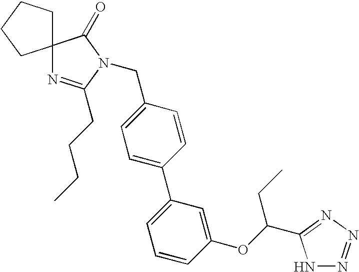

Prepared following the general procedure previously described (Method 15F) using 2-butyl-1-[(3′-((1-(1-benzyloxymethyltetrazol-5-yl)-1-ethylmethyl)oxy)-biphenyl-4-yl)methyl]-4-spirocyclopentyl-1H-imidazol-5(4H)-one (example 14.48). The product was chromatographed over silica gel (eluent dichloromethane/methanol 90/10). The product was obtained as a white solid.

Yield: 93%

Rf (dichloromethane/methanol 90/10): 0.2

MP: 76° C.

IR: vCO 1633 cm⁻¹

NMR ¹H (DMSO): 0.74 (t, 3H, J=7 Hz); 1.08 (t, 3H, J=7 Hz); 1.18-1.27 (m, 2H); 1.42-1.51 (m, 2H); 1.90-2.04 (m, 8H); 2.10-2.25 (m, 2H); 2.30-2.36 (m, 2H); 4.70 (s, 2H); 5.69 (t, 1H, J=7 Hz); 6.85 (d, 1H, J=7.3 Hz); 7.09 (m, 5H); 7.43 (d, 2H, J=7.8 Hz); 10.40 (s, 1H).

MS (ESI): 485 (M−H)

### Example 16

### In vitro Evaluation of the PPAR Activating Properties of the Compounds According to the Invention

The PPAR-activating properties of the compounds according to the invention were evaluated in vitro.

Principle

The activation of PPARs was evaluated in vitro using a monkey kidney fibroblast line (COS-7) by measuring the transcriptional activity of chimeras made up of the DNA binding domain of the Gal4 transcription factor of yeast and of the binding domain to the ligand of the different PPARs. The compounds were tested at doses of between 0.01 and 100 μM on Gal4-PPARα, γ, and δ chimeras.

Protocol

Culture of the Cells

COS-7 cells came from ATCC (American type culture collection) and were cultivated in a DMEM medium supplemented with 10% (vol/vol) of fetal calf serum, 100 U/ml of penicillin (Gibco, Paisley, UK) and 2 mM of L-Glutamine (Gibco, Paisley, UK). The cells were incubated at 37° C. under humid atmosphere containing 5% CO₂.

Description of the Plasmids Used in Transfection

The plasmids Gal4(RE)_TkpGL3, pGal4-hPPARα, pGal4-hPPARγ, pGal4-hPPARδ and pGal4-φ have been described in the literature (Raspe E et al., 1999). The constructions pGal4-hPPARα, pGal4-hPPARγ, and pGal4-hPPARδ were obtained by cloning, in the pGal4-φ vector, DNA fragments amplified by PCR and corresponding to the DEF domains of human PPARα, PPARγ, and PPARδ nuclear receptors.

Transfection

The COS-7 cells in suspension were transfected with 150 ng of DNA per well, with a pGal4-PPAR/Gal4(RE)_TkpGL3 ratio of 1/10, in presence of 10% fetal calf serum. The cells were then plated in 96-well plates (4×10⁴ cells/well), then incubated for 24 hours at 37° C. Activation with the test compounds was performed for 24 hours at 37° C. in a medium without serum. At the end of the experiment, the cells were lysed and the luciferase activity was determined using the Steady-Lite™ HTS (Perkin Elmert) or the Steady Glow Luciferase kit (Promega), in accordance with the provider's recommendations.

Results

### Example 16.1

### Transactivation of pGal4-hPPAR (α/γ/δ) in Function of the Dose

The compounds according to the invention were tested at doses between 0,01 and 100 μM on the 3 PPAR isoforms. The results obtained are presented in detail under FIG. 1a.

The inventors have shown a remarkable and dose-dependent increase in the luceferase activity in cells transfected pGal4-hPPAR and treated with the compounds according to the invention.

FIG. 1a shows the dose-dependent feature of this specific affinity. For example, compound 1 has an EC50 of 3μM for hPPARα and 8 μM hPPARγ, and compound 21 has an EC50 of 0.6 μM for hPPARα and an EC50 of 1 μM for HPPARγ.

FIG. 1b discloses EC50 relative to compounds according to the invention for hPPARα and hPPARγ. The lower the EC50 is, the stronger the affinity of the compound according to the invention for the receptor. Surprisingly, the compounds according to the invention bind to PPARs and activate them with high affinity.

Some compounds (for example, compound 1) activate the two PPAR isoforms, whereas others do activate one of them only (for example, compound 25 activates only hPPARα).

Conclusions

These results show that compounds according to the invention bind and activate hPPARα and/or hPPARγ significantly. The levels of transactivation achieved with compounds according to the invention are variable and depend on the structure of the tested compound and the studied PPAR subtype.

### Example 17

### In vitro Evaluation of the Binding between the Compounds According to the Invention and the Angiotensin II AT1 Receptor

Principle

The results presented the specific binding of the compounds according to the invention on the angiotensin II AT1 receptor, a well-known target of the antihypertensive medications currently sold on the market. IC50 stands for the concentration of the compound according to the invention which is needed to inhibit 50% of the binding of the reference molecule (saralasine). The lower the IC50 is, the stronger the affinity of the compound for the AT1 receptor

Protocol

The binding test was performed at the CEREP (Celle L'Evescault, France (86)) following a protocol based on Bergsma et al. (Bergsma D J et al., 1992): the binding was performed on recombinant CHO cells expressing the human AT1 receptor. The reference compound was [¹²⁵I][SaR1, Ile⁸]-ATII at 0.05 nM. The protocol consisted in an incubation at 37° C. for 60 minutes. Measurements were carried out using scintillation counting.

Results

### Example 17.1

### Binding of the Compounds According to the Invention to the Human Angiotensin II AT1 Receptor, in Function of the Dose

The compounds according to the invention were tested at doses between 0.001 and 10 μM on human angiotensin II AT1 receptor.

The inventors have shown a significant and dose-dependent augmentation in the binding of the compounds according to the invention to human angiotensin II AT1 receptor. FIG. 2 shows the IC50 (50% inhibitory concentration) of compounds according to the invention regarding human angiotensin II AT1 receptor. The lower the IC50 is, the stronger the affinity of the compound for AT1 receptor

Conclusion

The results disclosed show that the compounds according to the invention bind to human angiotensin II AT1 receptor significantly and dose-dependently. The levels of binding of the compounds according to the invention are variable and depend on the nature of the groups of compounds (e.g. according to the nature of groups L1, L2, R1, R2, E, etc.).

### Example 18

### Ex vivo Evaluation of the Antagonist Effect of the Compounds According to the Invention on the Angiotensin II AT1 Receptor

Principle

The disclosed results show the activity of the compounds according to the invention on the angiotensin II AT1 receptor. This activity is either agonist or antagonist and was evaluated ex vivo on isolated organs. Agonist activity corresponds to tissue contraction. Antagonist activity corresponds to tissue dilatation.

Protocol

The ex vivo test was performed at the CEREP (Celle L'Evescault, France (86)) following a protocol based on Pendleton et al. (Pendleton R G et al.,1989). Rabbit thoracic aorta rings with intact endothelium were suspended in baths for organs containing an oxygenated physiologic saline solution (95% O₂ and 5% CO₂) and preheated (37° C.) having the following composition (mM): NaCl 118.0, KCl 4.7, MgSO₄ 1.2, CaCl₂ 2.5, KH₂PO₄ 1.2, NaHCO₃ 25.0, and glucose 11.0 (pH 7.4).

Benextramine (1 μM), propranolol (1 μM), pyrilamine (1 μM), atropine (1 μM), methysergide (1 μM), and captopril (0.1 μM) were also used in all carried out experiments to block respectively α-adrenergic, β-adrenergic, histaminic H1 muscarinic, and 5-HT2 receptors as well as to prevent peptide degradation. The tissues were bound to a force sensor suitable for isometric tension recording. They were stretched to a resting tension of 4 g, then stabilized for 60 minutes. During the stabilization, they were washed several times and the tension was adjusted. The experiments were carried out using semi-automatic systems of isolated organs with eight organ baths and multichannel data acquisition.

Agonist Activity

The tissues were exposed to a submaximal concentration of the reference agonist (angiotensin II at 0.003 μM) to verify the response and obtain a control contractile response.

After several washing operations and after having recovered the basal tone, the tissues were exposed to increasing concentrations of compounds according to the invention or the same agonist. The different concentrations were added cumulatively, each one was left in contact with the tissues until a stable response was reached and for a maximum of 30 minutes. If an agonist type response (contraction) was registered, the reference antagonist (Saralasine at 0.01 μM) was tested regarding the highest concentration of the compounds according to the invention to confirm the involvement of the AT1 receptors in the response.

Antagonist Activity

The tissues were exposed to a submaximal concentration of the reference agonist (angiotensin II at 0.003 μM) to verify the response and obtain a control contractile response.

After stabilization of the contraction induced by angiotensin II, increasing concentrations of compounds according to the invention or of the reference antagonist (Saralasine) were added cumulatively. Each concentration was left in contact with the tissues until a stable response was observed and for a maximum of 30 minutes.

The inhibiting effect of the compounds according to the invention on the contraction induced by the angiotensin 11 indicates antagonist activity on AT1 receptors.

Results

The results disclosed, expressed in percentages, show the effects of compounds 1, 21, 53 and 80 according to the invention tested as agonists and antagonists of human angiotensin II AT1 receptor, on rabbit thoracic aorta. The measured parameter is the maximal change in tension induced by each compound concentration. The results are expressed in percentages of the control response to angiotensin II. On non-treated tissue, compounds 1, 21, 53 and 80 do not induce contraction. On tissue previously exposed to angiotensin II, compounds 1, 21, 53 and 80 according to the invention show an inhibiting effect of the contractile response of the reference agonist.

Therefore, the results show that the compounds according to the invention do not present agonist activity on the human angiotensin II AT1 receptor (FIG. 3a) and that compounds according to the invention are human AT1 angiotensin II receptor antagonists in a dose-dependent way (FIG. 3b).

Conclusion

The inventors have surprisingly shown the antagonist activity of the compounds according to the invention on the human angiotensin II AT1 receptor.

### Example 19

### In vivo Evaluation of the Compounds According to the Invention for Their Hypolipemic Properties and Their Capacity to Stimulate HDL-Cholesterol Synthesis, in the ApoE2/E2 Mouse

Principle

The hypolipemic properties of the compounds according to the invention were evaluated in vivo by assaying plasma lipids and by an analysis of the gene expression of PPARs target genes, in the liver after a treatment of the dyslipidemic E2/E2 mice with the compounds according to the invention.

The murine model used is the ApoE2/E2 mouse, a transgenic mouse having human apolipoprotein E isoform E2 (Sullivan P M et al., 1998). In human, this apolipoprotein, a constituent of the low and the very low density lipoproteins (LDL-VLDL), exists in three isoforms E2, E3, and E4. The E2 form has a mutation affecting the amino acid of position 158, which considerably weakens the affinity of this protein for the receptors to LDL., the VLDL clearance is nearly non-existent.

An accumulation of low-density lipoproteins then occurs along with a mixed hyperlipidemia known as of type III (high cholesterol and triglycerides rates). PPARα regulates the expression of genes involved in the transport of lipids (apolipoproteins such as Apo AI, Apo AII, and Apo CIII, membrane transporters such as FAT) or in the catabolism of lipids (ACO, CPT-I, or CPT-II, fatty acid β-oxidation enzymes). Accordingly, a treatment with PPARα activators, in human as well as in rodents, leads to a reduction in circulating triglycerides levels. Measuring the plasmatic lipids rate, after a treatment with the compounds according to the invention, allows an evaluation of the PPAR agonist properties of the compounds according to the invention, and consequently their hypolipemic effects.

The agonist properties of PPARα previously measured in vitro should, in the liver, lead to an over-expression of the target genes which are target genes directly under the control of the PPARα: the genes that were studied in these experiment are those coding for ACO (acyl co-enzyme A oxydase, a key enzyme in the mechanism of fatty acid β-oxidation), Apo CIII (apolipoprotein involved in lipid metabolism), and PDK-4 (pyruvate deshydrogenase kinase isoform 4, an enzyme of glucid metabolism).

In parallel, the treatment of animals with a PPARγ agonist should lead, in the white adipose tissue, to an over-expression of target genes which are directly under the control of PPAR: the gene studied in this experiment is the one coding for PEPCK (PhosphoEnolPyruvate CarboxyKinase, a neoglucogenesis enzyme).

Measuring the transcriptional activity of PPAR target genes after a treatment with compounds according to the invention, also inform about the hypolipidemic properties of the compounds according to the invention.

Protocol

Treatment of the Animals

The ApoE2/E2 transgenic mice were kept on a 12 hour/12 hour light/dark cycle at a constant temperature of 20±3° C. After a one week acclimatization period, the mice were weighed and divided into groups of 5 animals selected so as to render uniform the distribution of their body weights and their plasma lipid levels, determined before the experiment. The tested compounds were suspended in carboxymethylcellulose (Sigma C4888) and administered by intra-gastric tube feeding, once a day for 7 days at the chosen dose. The animals had free access to food and water. At the end of the experiment, the animals were anesthetized after a 4 hour fast, a blood sample was taken using anticoagulant (EDTA), then the mice were weighed and euthanized. The plasma was separated by centrifugation at 3000 rotations/minute for 20 minutes. The samples were kept at +4° C.

The liver and epididymal adipose tissue samples were taken and frozen immediately in liquid nitrogen then conserved at −80° C. for later analysis.

Measurement of Plasma Lipids

Plasma lipid concentrations (total cholesterol and triglycerides) were measured by enzymatic assays (bioMerieux-Lyon-France) according to the provider's recommendations.

Analysis of Cholesterol Distribution in Plasma Lipoprotein Fractions

The different lipid fractions (VLDL, LDL,HDL) of the plasma were separated by gel-filtration chromatography. The concentrations of cholesterol of each fraction were then measured by enzymatic assays (bioMerieux-Lyon-France) according to the provider's recommendations.

Gene Expression Analysis by Quantitative RT-PCR

Hepatic Tissue

Total RNA was extracted from liver fragments by using a NucleoSpin® 96 RNA kit (Macherey Nagel, Hoerdt, France) according to the manufacturer's instructions. 1 μg of total RNA (quantified by using the Ribogreen RNA quantification kit (Molecular Probes)) was then reverse-transcribed into cDNA by means of a 1 hour reaction at 37° C. in a total volume of 20 μl containing a buffer 1× (Sigma), 1.5 mM of DTT, 0.18 mM, of dNTPs (Promega), 200 ng of pdN6 (Amersham), 30 U of RNase inhibitor (Sigma), and 1 μl of MMLV-RT (Sigma).

Adipose Tissue

The total RNA of the adipose tissue was extracted from tissue fragments with a method using guanidine thiocyanate. The tissues were briefly homogenized in 5 mL of a lysis buffer (guanidine thiocyanate 4M, EDTA pH8 10 mM, Tris HCl pH 7.5 50 mM and b-mercaptoethanol 1.4%) using polytron. To separate the layers, 500 μL of sodium acetate 2M pH4, 5 mL of phenol, and 2 mL of a mixture of chloroform/isoamylic alcohol (49:1) were added. After centrifugation, the aqueous layer was collected and the RNA was precipitated in the presence of isopropanol. After a second precipitation, the RNA was washed with ethanol 70°, dried, then suspended again in an volume of water free of RNase. A step of purification/DNase I treatment of the RNA was then carried out by using NucleoSpin® 96 RNA kit (Macherey-Nagel) according to the manufacturer's instructions.

1 μg of total RNA (quantified by using the Ribogreen RNA quantification kit (Molecular Probes)) was then reverse-transcribed into cDNA by means of a 1 hour reaction at 37° C. in a total volume of 20 μl containing a buffer 1× (Sigma), 1.5 mM of DTT, 0.18 mM, of dNTPs (Promega), 200 ng of pdN6 (Amersham), 30 U of RNase inhibitor (Sigma), and 1 μl of MMLV-RT (Sigma).

The quantitative PCR experiments were carried out using the MyiQ Single-Color Real-Time PCR Detection System (Biorad, Marnes-la-Coquette, France) and were performed using the iQ SYBR Green Supermix kit according to the manufacturer's recommendations, in 96-well plates in 5 μl of a diluted reverse transcription solution at a hybridization temperature of 55° C. Primer pairs specific to the studied genes were used:

| | |
|---|---|
| PDK4: sense primer: | |
| 5′-TACTCCACTGCTCCAACACCTG-3′ | (SEQ ID NO: 1) |
| and | |
| antisense primer | |
| 5′-GTTCTTCGGTTCCCTGCTTG-3′ | (SEQ ID NO: 2) |
| ACO: sense primer: | |
| 5′-GAAGCCAGCGTTACGAGGTG-3′ | (SEQ ID NO: 3) |
| and | |
| antisense primer | |
| 5′-TGGAGTTCTTGGGACGGGTG-3′ | (SEQ ID NO: 4) |
| ApoCIII: sense primer: | |
| 5′-CTCTTGGCTCTCCTGGCATC-3′ | (SEQ ID NO: 5) |
| and | |
| antisense primer | |
| 5′-GCATCCTGGACCGTCTTGGA-3′ | (SEQ ID NO: 6) |
| PEPCK: sense primer: | |
| 5′-AAGGAAAACGCCTTGAACCT-3′ | (SEQ ID NO: 11) |
| and | |
| antisense primer | |
| 5′-GTAAGGGAGGTCGGTGTTGA-3′. | (SEQ ID NO: 12) |

In both cases (hepatic tissue and adipose tissue), the quantity of emitted fluorescence is directly proportional to the quantity of cDNA present at the beginning of the reaction and amplified during the PCR. For each target studied, a range of solutions is performed with successive dilutions of a mixture made up of a few μl of different reverse-transcription solutions. The relative levels of expression of each target are thus determined by using efficiency curves obtained with the points relative to the range.

The expression levels of the genes of interest were then normalized with respect to the level expression of the reference gene

36B4, in hepatic tissue (whose specific primers
are: sense primer: 5′-CATGCTCAACATCTCCCCCTTCTCC-3′
(SEQ ID NO: 15) and antisense primer: 5′-GGGAAGGTG
TAATCCGTCTCCACAG-3′ (SEQ ID NO: 16)),
18S, in adipose tissue (whose specific primers
are: sense primer: 5′-CGGACACGGACAGGATTGACAG-3′
(SEQ ID NO: 17) and antisense primer: 5′-AATCTCGGG
TGGTGGCTGAACGC-3′ (SEQ ID NO: 18)).

The induction factor, i.e. the ratio between the relative signal (induced by the compound according to the invention) and the average of relative values obtained with the control group, was then calculated for each sample. The higher the induction factor is, the more the compound promotes gene expression. The final result is represented as the average of the induction values within each experimental group.

Results

Measurement of Plasma Lipids

The results relative to FIGS. 4a and 4b, 5a and 5c show a remarkable dose-dependently decrease in total cholesterol and triglyceride level, after 7 days of treatment with compound 1 administered at 25, 50, 100 and 200 mpk, or with compound 21 administered at 10, 30 or 100 mpk.

FIG. 5b show that a 7-day treament with compound 21 leads to a modification in the cholesterol distribution in the lipoprotein fraction, with a significant decrease in the VLDL and LDL fractions and a significant increase in the HDL-cholesterol fraction.

Analysis of Gene Expression, by Quantitative RT-PCR

The results presented in FIGS. 4c, 4d 5d and 5e show that the compounds according to the invention induce a significant increase in hepatic expression of the gene coding for PDK-4, and a significant increase in hepatic expression of the gene coding for ACO. The disclosed results of FIG. 4e show that the compounds according to the invention induce a significant decrease in hepatic expression of ApoCII. The results presented in FIG. 4f show that the compounds according to the invention induce a significant and dose-dependent increase in the expression of the gene coding for PEPCK in adipose tissue.

Conclusion

The inventors have shown that the compounds according to the invention have hypolipemic properties, decreasing the plasma cholesterol and triglyceride rates. The compounds according to the invention have also the aptitude to increase the beneficial HDL-cholesterol fraction. Additionally, the inventors have shown that the compounds according to the invention are regulator of the expression of genes coding for enzymes highly involved in lipid and glucid metabolism. These results, obtained in vivo, demonstrate the therapeutic potential of the compounds according to the invention for major pathologies such as dyslipidemias.

### Example 20

### In vivo Evaluation of the Compounds According to the Invention for Their Antidiabetic and Hypolipemic Properties, in the db/db Mouse

Principle

The insulin-resistance and hypolipemic properties of the compounds according to the invention were evaluated in vivo by assaying the plasma lipid, by measuring the plasma glucose and insulin levels, and by an analysis of the gene expression of PPAR target genes, after a per os treatment with the compounds according to the invention. These tests were performed in the db/db mouse

Protocol

Treatment of the Animals

Female db/db mice were kept on a 12 hour/12 hour light/dark cycle at a constant temperature of 20±3° C. After a one week acclimatization period, the mice were weighed and divided into groups of 8 animals selected so as to render uniform the distribution of their body weights and their plasma lipid rates, determined before the experiment. The tested compounds were suspended in carboxymethylcellulose (Sigma C4888) and administered by intra-gastric tube feeding once a day for 28 days at the chosen dose. The animals had free access to food and water (standard diet). Taking of food and weight gain are recorded throughout the experiment. At the end of the experiment, the animals were anesthetized after a 4 hour fast, a blood sample was taken using (EDTA) anticoagulant, then the mice were weighed and euthanized. The plasma was separated by centrifugation at 3000 rotations/minute for 20 minutes. The samples were kept at +4° C.

Samples of hepatic tissue and adipose epididymal tissue were taken and frozen immediately in liquid nitrogen then conserved at −80° C. for later analysis.

Measurement of Plasma Lipids

Plasma lipid concentrations (total cholesterol and triglycerides) were measured by enzymatic assays (bioMerieux-Lyon-France) according to the provider's recommendations.

Measurement of Plasma Glycemia and Insulinemia

Murine plasma glucose was measured according to an enzyme-colorimetric method using a Glucose RTU kit (Biomerieux). Glucose is transformed into gluconic acid under the action of glucose oxidase; the reaction releases hydrogen peroxide. Hydrogen peroxide was measured according to the Trinder reaction which, under the action of a peroxidase and in the presence of phenol and amino-4-antipyrine, produces water and a colored product, quinoneimine. The color intensity, due to the quinoneimine, is proportional to the amount of glucose present in the sample.

Murine insulin is measured using ELISA method (using the INSKR020 kit from provider Crystal chem.). A microplate is coated with a mouse anti-insulin antibody. Then, the serum to be assayed for insulin is placed onto the plate. A guinea pig anti-insulin antibody is used to recognize the complex formed by the mouse insulin and the anti-insulin monoclonal antibody. Finally an anti-guinea pig antibody labeled with peroxidase is added and bind to the guinea pig anti-insulin antibody. The colorimetric reaction is performed by adding an OPD (Ortho Phenyl Diamine) enzyme substrate. The intensity of the color is proportional to the amount of insulin present in the sample.

Analysis of the Gene Expression, by Quantitative RT-PCR

Hepatic Tissue

Total RNA was extracted from liver fragments by using a NucleoSpine 96 RNA kit (Macherey Nagel, Hoerdt, France) according to the manufacturer's instructions.

Adipose Epididymal Tissue

The total RNA of the adipose tissue was extracted from tissue fragments with a method using guanidine thiocyanate. The tissues were briefly homogenized in 5 mL of a lysis buffer (guanidine thiocyanate 4M, EDTA pH8 10 mM, Tris HCl pH 7.5 50 mM and b-mercaptoethanol 1.4%) using polytron. To separate the layers, 500 μL of sodium acetate 2M pH4, 5 mL of phenol, and 2 mL of a mixture of chloroform/isoamylic alcohol (49:1) were added. After centrifugation, the aqueous layer was collected and the RNA was precipitated in the presence of isopropanol. After a second precipitation, the RNA was washed with ethanol 70°, dried, then suspended again in an volume of water free of RNase. A step of purification/DNase I treatment of the RNA was then carried out by using NucleoSpin® 96 RNA kit (Macherey-Nagel) according to the manufacturer's instructions. 1 μg of total RNA (quantified by using the Ribogreen RNA quantification kit (Molecular Probes)) was then reverse-transcribed into cDNA by means of a 1 hour reaction at 37° C. in a total volume of 20 μl containing a buffer 1× (Sigma), 1.5 mM of DTT, 0.18 mM, of dNTPs (Promega), 200 ng of pdN6 (Amersham), 30 U of RNase inhibitor (Sigma), and 1 μl of MMLV-RT (Sigma). The quantitative PCR experiments were carried out using the MyiQ Single-Color Real-Time PCR Detection System (Biorad, Marnes-la-Coquette, France) and were performed using the iQ SYBR Green Supermix kit according to the manufacturer's recommendations, in 96-well plates in 5 μl of a diluted reverse transcription solution at a hybridization temperature of 55° C. Primer pairs specific to the studied genes were used:

| | |
|---|---|
| PDK4: sense primer: | |
| 5′-TACTCCACTGCTCCAACACCTG-3′ | (SEQ ID NO: 1) |
| and | |
| antisense primer | |
| 5′-GTTCTTCGGTTCCCTGCTTG-3′ | (SEQ ID NO: 2) |
| CPT1b: sense primer: | |
| 5′-GGACTGAGACTGTGCGTTCCTG-3′ | (SEQ ID NO: 7) |
| and | |
| antisense primer: | |
| 5′-AGTGCTTGGCGGATGTGGTT-3′ | (SEQ ID NO: 8) |
| ApoCIII: sense primer: | |
| 5′-CTCTTGGCTCTCCTGGCATC-3′ | (SEQ ID NO: 5) |
| and | |
| antisense primer: | |
| 5′-CGATCCTGGACCGTCTTGGA-3′ | (SEQ ID NO: 6) |
| FGb: sense primer: | |
| 5′-AAGAAGATGGTGGTGGCTGGTG-3′ | (SEQ ID NO: 9) |
| and | |
| antisense primer | |
| 5′-GGGACTATTGCTGTGGGAAG-3′ | (SEQ ID NO: 10) |
| PEPCK: sense primer: | |
| 5′-AAGGAAAACGCCTTGAACCT-3′ | (SEQ ID NO: 11) |
| and | |
| antisense primer | |
| 5′-GTAAGGGAGGTCGGTGTTGA-3′. | (SEQ ID NO: 12) |

The quantity of emitted fluorescence is directly proportional to the quantity of cDNA present at the beginning of the reaction and amplified during the PCR. For each target studied, a range of solutions is performed with successive dilutions of a mixture made up of a few μl of different reverse-transcription solutions. The relative levels of expression of each target are thus determined by using efficiency curves obtained with the points relative to the range.

The expression levels of the genes of interest were then normalized, in the hepatic tissue with respect to the level expression of reference gene 36B4 (whose specific primers are: sense primer: 5′-CATGCTCAACATCTCCCCCTTCTCC-3′ (SEQ ID NO: 15) and antisense primer: 5′-GGGMGGTGTMTCCGTCTCCACAG-3′ (SEQ ID NO: 16)), and in the adipose tissue with respect to reference gene 18S (whose specific primers are: sense primer: 5′-CGGACACGGACAGGATTGACAG-3′ (SEQ ID NO: 17) and antisense primer: 5′-MTCTCGGGTGGTGGCTGMCGC-3′ (SEQ ID NO: 18)). The induction factor was then calculated for each sample. The higher the induction factor is, the more the compound promotes gene expression. The final result is represented as the average of the induction values within each experimental group.

Results

Measurement of Plasma Lipids

FIGS. 6a and 6b, 7a and 7b allow a comparison of the plasma triglycerides and free lipids levels after a 28-day treatment with compound 1 adiministered at 10, 30 or 100 mpk, and with compound 21 administered at 100 mpk, and the levels obtained with the control animals. Unexpectedly, triglycerides and free lipids levels decreased significantly (and dose-dependently) thanks to the treatment with compounds according to the invention.

Measurement of Glycemia and Insulinemia

FIGS. 6c, 6d, 7c and 7d allows a comparison of the plasma glucose and insulin levels after a 28-day treatment with compound 1 adiministered at 10, 30 or 100 mpk, and with compound 21 administered at 100 mpk, and the levels obtained with the control animals. Unexpectedly, glycemia and insulinemia decreased significantly (and dose-dependently) thanks to the treatment with the compounds according to the invention.

Analysis of Gene Expression, by Quantitative RT-PCR

The inventors have also shown that the compounds according to the invention are in vivo regulators of PPARs target genes expression. The results presented in FIGS. 6e to 6h and 7e to 7i show that compounds 1 and 2, administered at 50 mpk for 28 days, to db/db mice, induce, in liver, a significant increase in hepatic expression of the gene coding for PDK-4 (FIGS. 6f and 7f) and CPT1 b (FIGS. 6f and 7f) as well as a significant decrease in the expression of the gene coding for ApoCIII (FIGS. 6g ans 7g) and for FBb (FIGS. 6h and 7h). The experimental data also show that compounds according to the invention induce a significant increase in hepatic expression of the gene coding for PEPCK.

All these genes do code for enzymes which are highly involved in the lipid and glucid metabolisms, and in the anti-inflammatory response. The fact that their expression is regulated by the compounds according to the invention strengthens the idea that the compounds according to the invention have a main interest regarding the treatment of metabolic pathologies.

Conclusion

Unexpectedly, disclosed experimental data show that, in vivo, the compounds according to the invention improve the sensitivity to insulin and, in parallel, induce a hypolipemic effect (decrease of triglycerides and free lipids levels). Additionally, disclosed experimental data show that the compounds according to the invention modulate the expression of genes which are regulated by PPARs activation and which code for enzymes highly involved in lipid and glucid metabolisms, and in anti-inflammatory response.

### Example 21

### In vivo Evaluation of the Angiotensin II Antagonist Properties of the Compounds According to the Invention in Rats (Intravenous Administration)

The angiotensin II antagonist properties of compounds according to the invention were evaluated in vivo by intravenous administration on Wistar type normotensive rats.

Principle

This experimental procedure is intended to show the anti-hypertensive effects of the compounds according to the invention in Wistar rats in which hypertension is induced either by continuous intravenous administration of angiotensin II (example 21.1) or by repeated intravenous administration of angiotensin II (example 21.2). The animals were treated with the compounds according to the invention intravenously at increasing doses. To validate the study, the control animals were administered, in the same conditions, either the vehicle itself or a reference.

### Example 21.1

### In vivo Evaluation of the Angiotensin II Antagonist Properties of the Compounds According to the Invention in Rats (Intravenous Administration): Treatment of the Animal Under Angiotensin II Perfusion

Protocol

Wistar rats (males—200-250 g—5-6 weeks old—CERJ) were anesthetized for surgery using pentobarbital (50 mg/kg). A catheter was placed in the right jugular vein for the angiotensin II perfusion. A catheter was placed in the left jugular vein for the administration of the compounds according to the invention. The angiotensin II perfusion at 100 ng/kg/min was performed using an auto-syringe set at 5 ml/hour (vehicle NaCl 0.15M). A solution of Na₂CO₃/NaHCO₃ (50 mM pH 9.6) served as the vehicle for the compounds according to the invention. Arterial pressure was measured using a sensor set in the carotid artery via a catheter.

Results

The arterial pressure of the animal was measured for few minutes in basal conditions, before the angiotensin II perfusion was put into place. The average arterial pressure (P), expressed in mm of Hg, raised to a stable plateau. After being stabilized, successive injections of compounds according to the invention were performed. The parameters were followed in real time and an injection was performed when the previous injection reached its greatest effect.

Under angiotensin II perfusion, the compounds according to the invention at increasing doses showed an antagonist effect on angiotensin II in a significant and dose-dependent way as shown by the results obtained with compound 1 (FIG. 8a) and with compound 21 (FIG. 8b).

### Example 21.2

### In vivo Evaluation of the Angiotensin II Antagonist Properties of the Compounds According to the Invention in Rats (Intravenous Administration): Treatment of the Animals with Repeated Administration of Angiotensin II

Protocol

Wistar rats (males—200-250 g—5-6 weeks old—CERJ) were anesthetized for surgery using pentobarbital (50 mg/kg). A catheter was placed in the right jugular vein for angiotensin II administration. A catheter was placed in the left jugular vein the administration of the compounds according to the invention. Boluses of angiotensin II were performed at predetermined intervals by the experimenter using a manual syringe.

Results

The arterial pressure of the animal was measured for a few minutes at basal conditions; then three successive intravenous administrations of angiotensin II (50, 100, and 200 ng/kg) were performed. At every bolus of angiotensin II, the average arterial pressure, expressed in mm of Hg, increased in a very temporary way. The compounds according to the invention were then injected intravenously (20 mpk) and the cycle of 3 boluses of angiotensin II was repeated. The antihypertensive properties of the compounds according to the invention were evaluated by comparing the range of response to angiotensin II before and after administration of compounds according to the invention.

Under repeated administration of angiotensin II, the compounds according to the invention, at single dose, showed an antagonist effect on angiotensin II as compound 1 shows in FIG. 8c.

### Example 22

### In vitro Evaluation of the Cardioprotective Properties of the Compounds According to the Invention

Principle

The two previous examples (example 20 and example 21) showed that the compounds according to the invention have at least two pharmacological properties in vivo: angiotensin II antagonist and PPAR agonist. The goal of this study is to measure the therapeutic effect of the molecules according to the invention on a major pathology: arterial hypertension associated or not with a dyslipidemia.

Protocol

Treatment of the Animals

SHR rats (spontaneous hypertensive rats) (male sex—300-320 g—16-weeks old—Charles River France) were kept on a light/dark cycle of 12/12 hours at a constant temperature of 20±3° C. The compounds according to the invention being tested were suspended in carboxymethylcellulose (Sigma c4888) and administered to the animals by intra-gastric gavage, once a day for 14 days at the chosen doses. The animals had free access to food and water.

Measuring Arterial Pressure

At the end of the experiment, after a 5-hour fast, the animals were anesthetized for surgery with pentobarbital (50 mg/kg). A catheter treated with heparin was placed in the carotid artery to measure arterial pressure. A second catheter was placed in the jugular vein to administer angiotensin II. Arterial pressure was measured and recorded at basal tone for 15 minutes. At this stage, the average arterial pressure of the control animals is compared to that of the animals having received the compounds according to the invention. After 15 minutes, three successive intravenous administrations of angiotensin II (50 ng/kg) were performed to evaluate the response to angiotensin II of animals treated with the compounds according to the invention.

At the end of the arterial pressure measurements, a blood sample was taken using anticoagulant (EDTA), then the animals were euthanized. The plasma was separated by centrifugation at 3000 rotations/minute for 20 minutes. The samples were kept at +4° C.

The liver and epididymal adipose tissue samples were taken, weighed, and frozen immediately in liquid nitrogen then conserved at −80° C. for later analysis.

Measurement of Lipid Parameters

Plasma total concentrations of cholesterol, triglycerides, free fatty acids, and glucose were measured by enzymatic assays (bioMerieux-Lyon-France and Wako-Japan) according to the provider's recommendations.

Insulinaemias were determined by an ELISA Crystal Chem Inc-USA) method according to the provider's recommendations.

Analysis of Gene Expression by Quantitative RT-PCR

The gene expression analyses by quantitative RT-PCR were performed according to the previously described procedure (example 20).

Results

Measurement of Lipid Parameters

FIG. 9 shows triglyceride rates after 14 days of treatment with compound 1 at 150 mpk. The plasma triglyceride rate was unexpectedly decreased by the treatment.

Measuring Arterial Pressure

Before Repeated Intravenous Administrations of Angiotensin II

After a chronic treatment, the compounds according to the invention show an antihypertensive effect as shown by compound 1 at 150 mpk in FIG. 10a.

After Repeated Intravenous Administrations of Angiotensin II

After chronic treatment and repeated administrations of angiotensin II, the compounds according to the invention showed a significant antagonist effect on angiotensin II as shown by compound 1 at 150 mpk in FIG. 10b and compound 21 at 100 mpk in FIG. 10c: the decrease in hypertension induced by angiotensin II demonstrated the antagonist effect of the compounds according to the invention.

Analysis of Gene Expression by Quantitative RT-PCR

The results presented in FIGS. 11a and 11b show that the compounds according to the invention induce a significant increase in the hepatic expression of genes coding respectively for ACO and PDK-4.

Conclusion

The decrease in the plasma triglyceride rate demonstrates the hypolipemic properties of the compounds according to the invention. Moreover, the inventors have shown that the compounds according to the invention have antihypertensive properties.

These results, obtained in vivo on hypertensive rats, demonstrate the therapeutic potential of the compounds according to the invention for major pathologies such as hypertension associated or not with dyslipidemias.

### Example 23

### In vitro Evaluation of Anti-Inflammatory Properties of the Compounds According to the Invention in Monocytes

Principle

The anti-inflammatory effects of the compounds according to the invention were evaluated by measuring the secretion of MCP1 (Monocyte chemotactic protein-1) by THP1 monocytes treated for 24 hours with compounds according to the invention and stimulated simultaneously with PMA (Phorbol 12-myristate 13-acetate, which promotes an inflammatory response in cells and their differentiation into macrophages). The less MCP-1 is secreted, the more the compound according to the invention inhibits the inflammatory reaction.

Protocol

Culture and Treatment of THP-1 Cells

THP-1 human monocytes line (ATCC source) is cultured in a RPMI1640 medium supplemented with 25 mM Hepes (Gibco; 42401-018), 1% glutamine (Gibco; 25030-24) 1% penicillin/streptomycin (Biochrom AG; A 2213), and 10% decomplemented fetal calf serum (SVF. Gibco; 26050-088).

The cells were plated in 24-well plates (Primaria BD Falcon) at a density of 870,000 cells/well then were incubated at 37° C. and 5% CO₂ for 24 hours in a culture medium containing 0.2% fetal calf serum in the presence of 5 ng/ml of phorbol 12-myristate 13-acetate (PMA) and 1 μM of compound 8 according to the invention. The compound according to the invention is dissolved in dimethyl sulfoxide (DMSO, Fluka; 41640). The effect of the compounds according to the invention is compared to the effect of the DMSO alone.

RNA Extraction, Reverse Transcription and Quantitative PCR

After treatment, total RNA was extracted from cells by using a NucleoSpin® 96 RNA kit (Macherey Nagel, Hoerdt, France) according to the manufacturer's instructions.

1 μg of total RNA (quantified by spectrophotometer reading) was then reverse-transcribed into cDNA by a 1-hour reaction at 37° C. in a total volume of 20 μl containing a buffer 1× (Sigma), 1.5 mM of DTT, 0.18 mM, of dNTPs (Promega), 200 ng of pdN6 (Amersham), 30 U of RNase inhibitor (Sigma), and 1 μl of MMLV-RT (Sigma).

The quantitative PCR experiments were carried out using the MyiQ Single-Color Real-Time PCR Detection System (Biorad, Marnes-la-Coquette, France) and were performed using the iQ SYBR Green Supermix kit according to the manufacturer's recommendations, in 96-well plates in 5 μl of a diluted reverse transcription solution at a hybridization temperature of 55° C. Primer pairs specific to the studied genes were used:

| | |
|---|---|
| MCP1: sense primer: | |
| 5′-AGTCTTCGGAGTTTGGGTTTG-3′ | (SEQ ID NO: 13) |
| and | |
| antisense primer: | |
| 5′-AGGAAGATCTCAGTGCAGAGG-3′ | (SEQ ID NO: 14) |

The quantity of emitted fluorescence is directly proportional to the quantity of cDNA present at the beginning of the reaction and amplified during the PCR. For each target studied, a range of solutions is performed with successive dilutions of a mixture made up of a few μl of different reverse-transcription solutions. The relative levels of expression of each target are thus determined by using efficiency curves obtained with the points relative to the range.

The expression levels of the genes of interest were then normalized, in the hepatic tissue with respect to the level expression of reference gene 36B4 (whose specific primers are: sense primer: 5′-CATGCTCAACATCTCCCCCTTCTCC-3′ (SEQ ID NO: 15) and antisense primer: 5′-GGGMGGTGTMTCCGTCTCCACAG-3′ (SEQ ID NO: 16)).

The induction factor was then calculated for each sample. The higher the induction factor is, the more the compound promotes gene expression. The final result is represented as the average of the induction values within each experimental group.

Results

The inventors have shown that, on in vitro monocytes, the compounds according to the invention have anti-inflammatory effects. The results presented in FIG. 12 show that compound 3 according to the invention, at 10 μM, induces a significant reduction in the expression of MCP1 by monocytes.

Conclusion

Unexpectedly, the disclosed experimental data show that the compounds according to the invention have an anti-inflammatory effect on monocytes stimulated by PMA.

### Example 24

### In vitro Evaluation of Anti-Inflammatory Properties of the Compounds According to the Invention in Endothelial Cells

Principle

The anti-inflammatory effects of the compounds according to the invention were evaluated by measuring the secretion of MCP1 (Monocyte chemotactic protein-1), IL-8 (Interleukine 8) VCAM (Vascular Cell Adhesion Molecule) and ICAM (intracellular Adhesion Molecule-1) on endothelial cells of human umbilical vein (HUVEC) treated for 24 hours with the compounds according to the invention, and then stimulated for 24 hours by LPS (Lipopolysaccharide which induces an inflammatory response in the cells). The less the markers are secreted, the more the compound according to the invention inhibits the inflammatory reaction.

Protocol

Culture and Treatment of HUVEC Cells

The endothelial cells of human umbilical vein (HUVEC, from ATCC source) is cultured in an EBM medium (Endothelial Basal Media, CAMBREX; CC-3121) supplemented with EGM SingleQuots (CAMBREX; CC-4133, do not add gentamicine and Bovine Brain Extract [BBE]) and HE complement (Heparine 0.1 g/L final, [SIGMA; H3149], hECGS [Human endothelial Cells Growth Factors, Becton Dinckinson; 356006] 0.03 g/L final).

The cells were plated in 24-well plates (BD Biosciences; Biocoat 356408) at a density of 50 000 cells/well then were incubated at 37° C. and 5% CO₂ for 24 hours in a culture medium without HE complement and containing 10 or 50 μM of a compound according to the invention. 0.2% fetal calf serum in the presence of 5 ng/ml of phorbol 12-myristate 13-acetate (PMA) and 1 μM of compound 3 according to the invention. The compound according to the invention is dissolved in dimethyl sulfoxide (DMSO, Fluka; 41640). The effect of the compounds according to the invention is compared to the effect of the DMSO alone.

Measurement of the Secretion of Inflammation Markers

The treatment medium was taken and the concentration of markers measured by using the following sets:
Elisa “Human MCP-1 ELISA Set” (BD OptEIA; 555179) according to the provider's recommendations
Elisa “Human IL-8 ELISA Set” (BD OptEIA; 555244) according to the provider's recommendations
Elisa “Human VCAM-1” (R&D Biosciences; DY809) according to the provider's recommendations
Elisa “Human ICAM-1” (R&D Biosciences; DY809) according to the provider's recommendations.

The marker is set on a plate to be specifically recognized by an anti-marker antibody. Said antibody is itself recognized by a second antibody which is labeled with a peroxidase. The coloration resulting from the enzymatic reaction is proportional to the fixed marker amount, and can be measured by using a spectrophotometry method. A range is performed from a point representative of a known concentration and from which the MCP1 concentration of each sample is calculated. A is performed from a concentration point representative of a known concentration and from which the marker concentration of each sample is calculated. The final result is represented as the average of the induction values obtained with each experimental group. The induction factor, i.e. the ratio between the signal induced by the compound according to the invention and the signal induced by the control group, was then calculated. The weaker this factor is, the more the compound inhibits the secretion of the assayed marker.

Results

The inventors have shown, on in vitro HUVEC, that the compounds according to the invention have anti-inflammatory effects. The disclosed results in FIGS. 13a, 13b, 13c, 13d show that the compounds according to the invention, at 10 and 50 μM, induce a significant decrease in the secretion of inflammation markers (MCP1, IL8, VCAM, ICAM) by endothelial cells.

Coclusion

Unexpectedly, the disclosed experimental data show that the compounds according to the invention have an anti-inflammatory effect on endothelial cells (HUVEC) stimulated by PMA.

General Conclusion

The inventors have shown that the compounds according to the invention have hypolipemic properties (leading to a decrease in the plasma cholesterol and triglycerides levels) as well as antidiabetic properties (leading to a decrease in the plasma glucose and insulin rates). It has also been shown that, in vivo, the compounds according to the invention have the property to decrease the arterial pressure.

The inventors have also shown that the compounds according to the invention have anti-inflammatory properties.

Additionally, the inventors have shown that the compounds according to the invention are regulators of the expression of genes coding for enzymes highly involved in the metabolism of lipids and glucids, and in the inflammatory response.

All these results, obtained with in vivo and in vitro experiments, show the therapeutic potential of the compounds according to the invention for major pathologies such as dyslipidemias, type 2 diabetes and obesity.

### BIBLIOGRAPHY

Benson S C, Pershadsingh H A, Ho C I, Chittiboyina A, Desai P, Pravenec M, Qi N, Wang J, Avery M A and Kurtz T W, Identification of telmisartan as a unique angiotensin II receptor antagonist with selective PPARgamma-modulating activity, Hypertension, 2004, 43 (5), 993-1002

Bergsma D J, Ellis C, Kumar C, Nuthulaganti P, Kersten H, Elshourbagy N, Griffin E, Stadel J M and Aiyar N, Cloning and characterization of a human angiotensin II type 1 receptor, Biochem Biophys Res Commun, 1992,183 (3), 989-95

Bernhart C, Breliere J, Clement J, Nisato D, Perreault P, Muneaux C and Muneaux Y, N-substituted heterocyclic derivatives, their preparation and the pharmaceutical compositions in which they are present, 1993, (U.S. Pat. No. 5,270,317),

Bernhart C, Perreaut P, Ferrari B, Muneaux Y, Assens J, Clement J, Haudricourt F, Muneaux C, Taillades J and Vignal M, A new series of imidazolones: highly specific and potent nonpeptide AT1 angiotensin II receptor antagonists., J Med Chem, 1993, 36 (22), 3371-80

Black S L, Jales A R, Brandt W, Lewis J W and Husbands S M, The role of the side chain in determining relative delta- and kappa-affinity in C5′-substituted analogues of naltrindole, J Med Chem, 2003, 46 (2), 314-7

de Gasparo M, Inagami T, Wright J W and Unger T, International Union of Pharmacology. XXIII. The Angiotensin II Receptors, Pharmacol Rev, 2000, 52 (3), 415-472

Denke M A, Combination therapy, J Manag Care Pharm, 2003, 9 (1 Suppl), 17-9

Fox-Tucker J, The Cardiovasular Market Outlook to 2010, BUSINESS INSIGHTS REPORTS, 2005,1-174

International Atherosclerosis Society, Harmonized Clinical. Guidelines on Prevention of Atherosclerotic Vascular Disease, 2003,

Kintscher U, Lyon C J and Law R E, Angiotensin II, PPAR-gamma and atherosclerosis, Front Biosci, 2004, 9 359-69

Koh K K, Quon M J, Han S H, Chung W J, Ahn J Y, Kim J A, Lee Y and Shin E K, Additive beneficial effects of fenofibrate combined with candesartan in the treatment of hypertriglyceridemic hypertensive patients, Diabetes Care, 2006, 29 (2), 195-201

Kota B P, Huang T H and Roufogalis B D, An overview on biological mechanisms of PPARs, Pharmacol Res, 2005, 51 (2), 85-94

Kurtz T W, Treating the metabolic syndrome: telmisartan as a peroxisome proliferator-activated receptor-gamma activator, Acta Diabetol, 2005, 42 Suppl 1 S9-16

Lefebvre P, Chinetti G, Fruchart J C and Staels B, Sorting out the roles of PPARalpha in energy metabolism and vascular homeostasis, J Clin Invest, 2006, 116 (3), 571-580

McElwain S and Nelson J, The preparation of orthoesters, J Am Chem Soc, 1942, 64 (8), 1825-27

Mensah M, The Atlas of Heart Disease and Stroke, 2004,

Morphy R and Rankovic Z, Designed multiple ligands. An emerging drug discovery paradigm, J Med Chem, 2005, 48 (21), 6523-43

Pendleton R G, Gessner G and Horner E, Studies on inhibition of angiotensin II receptors in rabbit adrenal and aorta, J Pharmacol Exp Ther, 1989, 248 (2), 637-43

Raspe E, Madsen L, Lefebvre A M, Leitersdorf I, Gelman L, Peinado-Onsurbe J, Dallongeville J, Fruchart J C, Berge R and Staels B, Modulation of rat liver apolipoprotein gene expression and serum lipid levels by tetradecylthioacetic acid (TTA) via PPARalpha activation, J Lipid Res, 1999, 40 (11), 2099-110

Sawyer J S, Schmittling E A, Palkowitz J A and Smith W J, 3rd, Synthesis of Diaryl Ethers, Diaryl Thioethers, and Diarylamines Mediated by Potassium Fluoride-Alumina and 18-Crown-6: Expansion of Scope and Utility(1), J Org Chem, 1998, 63 (18), 6338-6343

Sullivan P M, Mezdour H, Quarfordt S H and Maeda N, Type III hyperlipoproteinemia and spontaneous atherosclerosis in mice resulting from gene replacement of mouse Apoe with human Apoe*2, J Clin Invest, 1998, 102 (1),130-5

Tsang J W, Schmied B, Nyfeler R and Goodman M, Peptide sweeteners. 6. Structural studies on the C-terminal amino acid of L-aspartyl dipeptide sweeteners, J Med Chem, 1984, 27 (12), 1663-8

Vera T, Taylor M, Bohman Q, Flasch A, Roman R J and Stec D E, Fenofibrate prevents the development of angiotensin II-dependent hypertension in mice, Hypertension, 2005, 45 (4), 730-5

Zou Y, Wang Q, Tao F and Ding Z, Palladium catalyzed Suzuki cross-coupling reaction in molten tetra-n-butylammonium bromide, Chin J Chem, 2004, 22 215-8

1- Compounds of general formula (I): 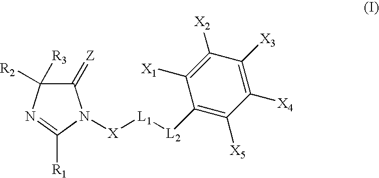
in which:
R1 represents a hydrogen or an alkyl, cycloalkyl, alkyloxy, alkylthio, alkenyl, alkynyl, aryl, arylalkyl, or heteroaryl group, or a heterocycle;
R2 and R3, identical or different, represent independently a hydrogen atom or an alkyl, alkenyl, alkynyl, cycloalkyl, aryl, or arylalkyl group, or a heterocycle, or R2 and R3, with the carbon they are bound to, can form a cycle or a heterocycle;
Z represents an oxygen atom or a sulfur atom;
X represents an alkyl group the principal chain of which comprises 1 to 6 carbon atoms or X represents an alkenyl or alkynyl group the principal chain of which comprises 2 to 6 carbon atoms;
L1 represents:
(i) a covalent bond, or
(ii) a heterocycle, or
(iii) a formula (II) group defined as follows: 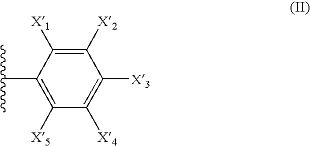
X′1, X′2, X′3, X′4, and X′5, identical or different, representing independently a hydrogen or halogen atom, a NO₂, nitrile, alkyl, cycloalkyl, alkenyl, alkynyl, aryl, arylalkyl, —OR₄, —SR4, —NR4R5, —SOR6, or —SO₂R6 group, a heterocycle, with one of X′1, X′2, X′3, X′4, or X′5 being L2;
L2 represents:
(i) a covalent bond, or
(ii) a carbonyl group (CO), or
(iii) an oxygen or sulfur atom, or
(iv) a methylene group (CH₂);
L1 and L2 cannot simultaneously represent a covalent bond if X has only 1 carbon atom;
X1, X2, X3, X4, and X5, identical or different, represent independently a hydrogen or halogen atom, a NO₂, nitrile, alkyl, cycloalkyl, alkenyl, alkynyl, aryl, arylalkyl, —OR4, —SR4, —NR4R5, —SOR6, a —SO₂R6 group, a heterocycle or a —Y-E group, with at least one of groups X1, X2, X3, X4, and X5 being a —Y-E group;
R4 and R5, identical or different, represent independently a hydrogen atom or an alkyl, cycloalkyl, alkenyl, alkynyl, aryl, or arylalkyl group, a heterocycle, or R4 and R5, together with the nitrogen atom to which they are bound, can form a heterocycle;
R6, substituted or not, represents independently an alkyl, cycloalkyl, alkenyl, alkynyl, aryl, or arylalkyl group, or a heterocycle;
Y represents a methylene group substituted or not, an oxygen, sulfur, or selenium atom, a SO, SO₂, SeO, SeO₂, or NR group, in which R represents a hydrogen atom or an alkyl, cycloalkyl, alkenyl, alkynyl, aryl, or arylalkyl group, or a heterocycle;
E represents an alkyl, cycloalkyl, alkenyl, or alkynyl chain, comprising or not one or several Y1 groups and substituted by one or several W groups,
Y1 represents an oxygen or sulfur atom, or NR type group, R representing a hydrogen atom or an alkyl, cycloalkyl, alkenyl, alkynyl, aryl, or arylalkyl group;
W represents:
(i) a carboxylic acid (—COOH) or an ester (—COOR4), a thioester (—COSR4) an amide (—CONR4R5), a thioamide (—CSNR4R5), a nitrile (—CN), or
(ii) an acylsulfonamide group (—CONHSO₂R6), or
(iii) a tetrazole, or
(iv) an isoxazole, or
(v) a sulfonic acid (—SO₃H), or
(vi) a —SO₃R4 or —SO₂NR4R5, or
(vii) a hydrazide (—CONHNR4R5),
R4, R5, and R6 being such as previously defined;
their stereoisomers (diastereoisomers, enantiomers), pure or mixed, racemic mixtures, geometrical isomers, tautomers, salts, hydrates, solvates, solid forms and mixtures thereof.

2- Compounds according to claim 1, characterized in that L1 represents a group of formula (II): 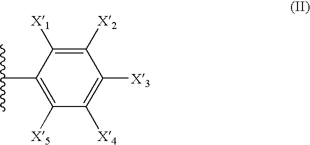
in which X′1, X′2, X′3, X′4, and X′5 are such as defined in claim 1.

3- Compounds according to the claim 2, characterized in that X′3 represents the L2 group.

4- Compounds according to claim 1, characterized in that X′1, X′2, X′4, and X′5 represent a hydrogen atom and X′3 represents the L2 group.

5- Compounds according to claim 1, characterized in that L2 represents a covalent bond.

6- Compounds according to claim 1, characterized in that L2 represents a carbonyl group (CO).

7- Compounds according to claim 1, characterized in that L2 represents an oxygen atom.

8- Compounds according to claim 1, characterized in that L2 represents a sulfur atom.

9- Compounds according to claim 1, characterized in that L2 represents a methylene group.

10- Compounds according to claim 1, characterized in that L1 represents a formula (X) group defined as follows: 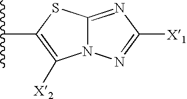
(X)
in which X′1, X′2 are as defined in claim 1.

11- Compounds according to claim 2, characterized in that X′2 represents the L2 group.

12- Compounds according to claim 1, characterized in that L1 and L2 simultaneously represent a covalent bond and X comprises more than one carbon atom.

13- Compounds according to claim 1, characterized in that R1 represents an alkyl group.

14- Compounds according to claim 1, characterized in that R2 and R3, identical or different, independently represent an alkyl group, an arylalkyl group, or R2 and R3 form a cycle with the carbon they are bound to.

15- Compounds according to claim 1, characterized in that Z represents an oxygen atom.

16- Compounds according to claim 1, characterized in that X represents an alkyl group in which the principal chain comprises 1 or 2 carbon atoms.

17- The compounds according to claim 1, characterized in that X1, X2, X3, X4, and X5, identical or different, independently represent a hydrogen atom, a halogen atom, an alkyl group, an alkoxy group, a nitrile group, a nitro group, or a —Y-E group, at least one of groups X1, X2, X3, X4, and X5 being a —Y-E group, said —Y-E group being such as defined in claim 1.

18- The compounds according to claim 1, characterized in that only one of the groups X1, X2, X3, X4, and X5 represents a —Y-E group, said Y-E group.

19- The compounds according to claim 18, characterized in that the Y-E group is in the meta position of the aromatic cycle to which it is attached.

20- Compounds according to claim 1, characterized in that Y represents an oxygen atom.

21- Compounds according to claim 1, characterized in that E represents an alkyl principal chain, branched or not, substituted by one or several W groups.

22- Compounds according to claim 1, characterized in that W represents a carboxylic acid (—COOH) or an ester (—COOR4), a thioester (—COSR4), an amide (—CONR4R5), a thioamide (—CSNR4R5), a nitrile (—CN), an acylsulfonamide (—CONHSO₂R6), a hydrazide (—CONHNR4R5), or a tetrazole, R4, R5, and R6.

23- Compounds according to claim 1, characterized in that the Y-E group represents —O—C(CH₃)₂—COOH, —O—(CH₂)₃—C(CH₃)₂—COOH, —O—CH₂—CN, —O—CH₂—C(CH₃)₂—COOH, —O—(CH₂)₆—C(CH₃)₂—COOH, —O—CH₂—COOH, —O—CH(CH₃)—COOH, —O—CH(CH₂CH₃)—COOH, —O—CH(CH(CH₃)₂)—COOH, —O—CH₂-tetrazole, —O—CH(CH₂CH₃)-tetrazole, —O(spirocyclobutyl)-COOH.

24- Compounds according to claim 1, characterized in that they are chosen among:
2-butyl-1-[(3′-((1-carboxy-1,1-dimethylmethyl)oxy)biphenyl-4-yl)methyl]-4-spirocyclopentyl-1H-imidazol-5(4H)-one;
2-butyl-1-[(4′-((1-carboxy-1,1-dimethylmethyl)oxy)biphenyl-4-yl)methyl]-4-spirocyclopentyl-1H-imidazol-5(4H)-one;
2-butyl-1-[2-(4-((1-carboxy-1,1-dimethylmethyl)oxy)phenyl)ethyl]-4-spirocyclopentyl-1H-imidazol-5(4H)-one;
1-[(5′-bromo-2′-((1-carboxy-1,1-dimethylmethyl)oxy)biphenyl-4-yl)methyl]-2-butyl-4-spirocyclopentyl-1H-imidazol-5(4H)-one;
2-butyl-1-[[4-[(3-((1-carboxyl-1,1-dimethylmethyl)oxy)phenyl)carbonyl]phenyl]methyl]4-spirocyclopentyl-1H-imidazol-5(4H)-one;
2-butyl-1-[(2′-((1-carboxy-1,1-dimethylmethyl)oxy)biphenyl-4-yl )methyl]-4-spirocyclopentyl-1H-imidazol-5(4H)-one;
2-butyl-1-[[4-[(2-((1-carboxyl-1,1-dimethylmethyl)oxy)phenyl)carbonyl]phenyl]methyl]-4-spirocyclopentyl-1H-imidazol-5(4H)-one;
2-butyl-1-[2-(3-((1-carboxy-1,1-dimethylmethyl)oxy)phenyl)ethyl]-4-spirocyclopentyl-1H-imidazol-5(4H)-one;
2-butyl-1-[[4-[(4-((1-carboxyl-1,1-dimethylmethyl)oxy)phenyl)carbonyl]phenyl]methyl]4-spirocyclopentyl-1H-imidazol-5(4H)-one;
2-butyl-1-[2-(2-((1-carboxy-1,1-dimethylmethyl)oxy)phenyl)ethyl]-4-spirocyclopentyl-1H-imidazol-5(4H)-one;
2-butyl-1-[[4-[(3-((1-carboxyl-1,1-dimethylmethyl)oxy)phenyl)carbonyl]phenyl]methyl]4-spirocyclohexyl-1H-imidazol-5(4H)-one;
1-[(6′-bromo-3′-((1-carboxy-1,1-dimethylmethyl)oxy)biphenyl-4-yl)methyl]-2-butyl-4-spirocyclopentyl-1H-imidazol-5(4H)-one;
2-butyl-1-[(3′-((1-carboxy-1,1-dimethylmethyl)oxy)biphenyl-4-yl)methyl]-4,4-dimethyl-1H-imidazol-5(4H)-one;
2-butyl-1-[[4-[(3-((1-carboxyl-1,1-dimethylmethyl)oxy)phenyl )carbonyl]phenyl]methyl]-4,4-dimethyl-1H-imidazol-5(4H)-one;
2-butyl-1-[[4-[(3-((1-carboxyl-1,1-dimethylmethyl)oxy)phenyl)carbonyl]phenyl]methyl]-4-phenyl-1H-imidazol-5(4H)-one;
1-[(3′-((1-carboxy-1,1-dimethylmethyl)oxy)biphenyl-4-yl)methyl]-2-propyl-4-spirocyclopentyl-1H-imidazol-5(4H)-one;
1-[(3′-((1-carboxy-1,1-dimethylmethyl)oxy)biphenyl-4-yl)methyl]-2-ethyl-4-spirocyclopentyl-1H-imidazol-5(4H)-one;
1-[(3′-((1-carboxy-1,1-dimethylmethyl)oxy)biphenyl-4-yl)methyl]-2-methyl-4-spirocyclopentyl-1H-imidazol-5(4H)-one;
2-butyl-1-[(3′-((1-carboxy-1,1-dimethylmethyl)oxy)biphenyl-4-yl)methyl]-4-phenyl-1H-imidazol-5(4H)-one;
2-butyl-1-[[4-[(4-((1-carboxyl-1,1-dimethylmethyl)oxy)phenyl)carbonyl]phenyl]methyl]-4-phenyl-1H-imidazol-5(4H)-one;
2-butyl-1-[(3′-((1-carboxy-1,1-dimethylmethyl)oxy)biphenyl-4-yl)methyl]4-spirocyclohexyl-1H-imidazol-5(4H)-one;
1-[(6′-bromo-3′-((1-carboxy-1,1-dimethylmethyl)oxy)biphenyl-4-yl )methyl]-2-butyl-4-spirocyclohexyl-1H-imidazol-5(4H)-one;
2-butyl-1-[(3′-(cyanomethoxy)biphenyl-4-yl)methyl]-4-spirocyclopentyl-1H-imidazol-5(4H)-one;
1-[(5′-bromo-2′-((1-carboxy-1,1-dimethylmethyl)oxy)biphenyl-4-yl)methyl]-2-butyl-4-spirocyclohexyl-1H-imidazol-5(4H)-one;
2-butyl-1-[(4′-((1-carboxy-1,1-dimethylmethyl)oxy)biphenyl-4-yl)methyl]-4-spirocyclohexyl-1H-imidazol-5(4H)-one;
1-[(5′-bromo-2′-((1-carboxy-1,1-dimethylmethyl)oxy)biphenyl-4-yl)methyl]-2-butyl-4-phenyl-1H-imidazol-5(4H)-one;
1-[(3′-((1-carboxy-1,1-dimethylmethyl)oxy)biphenyl-4-yl)methyl]-2-(2-methyl)propyl-4-spirocyclopentyl-1H-imidazol-5(4H)-one;
2-benzyl-1-[(3′-((1-carboxy-1,1-dimethylmethyl )oxy)biphenyl-4-yl )methyl]-4-spirocyclopentyl-1H-imidazol-5(4H)-one;
1-[(3′-((1-carboxy-1,1-dimethylmethyl)oxy)biphenyl-4-yl )methyl]-2-cyclopropyl-4-spirocyclopentyl-1H-imidazol-5(4H)-one;
1-[(3′-((1-carboxy-1,1-dimethylmethyl)oxy)biphenyl-4-yl)methyl]-2-(thiophen-2-yl)methyl-4-spirocyclopentyl-1H-imidazol-5(4H)-one;
2-butyl-1-[[4-[(4-((1-carboxy-1,1-dimethylmethyloxy)phenyl)methyl]phenyl]methyl]-4-spirocyclopentyl-1H-imidazol-5(4H)-one;
2-butyl-1-[(4′-((4-carboxy-4,4-dimethylbutan-1-yl)oxy)biphenyl-4-yl)methyl]-4-spirocyclopentyl-1H-imidazol-5(4H)-one;
2-butyl-1-[(3′-((4-carboxy-4,4-dimethylbutan-1-yl)oxy)biphenyl-4-yl )methyl]-4-spirocyclopentyl-1H-imidazol-5(4H)-one;
2-butyl-1-[[4-[(4-((1-carboxy-1,1-dimethylmethyloxy)phenyl)oxy]phenyl]methyl]4-spirocyclopentyl-1H-imidazol-5(4H)-one;
2-butyl-1-[[4-[(4-((1-carboxy-1,1-dimethylmethyloxy)phenyl)oxy]phenyl]methyl]-4-spirocyclohexyl-1H-imidazol-5(4H)-one;
2-butyl-1-[[4-[(3-((1-carboxy-1,1-dimethylmethyloxy)phenyl)methyl]phenyl]methyl]-4-spirocyclopentyl-1H-imidazol-5(4H)-one;
2-butyl-1-[[4-[(3-((1-carboxy-1,1-dimethylmethyloxy)phenyl)methyl]phenyl]methyl]-4-spirocyclohexyl-1H-imidazol-5(4H)-one;
2-butyl-1-[(4′-((4-carboxy-4,4-dimethylbutan-1-yl)oxy)biphenyl-4-yl)methyl]-4-spirocyclohexyl-1H-imidazol-5(4H)-one;
2-butyl-1-[[4-[(3-((1-carboxy-1 1-dimethylmethyloxy)phenyl)oxy]phenyl]methyl]4-spirocyclohexyl-1H-imidazol-5(4H)-one;
2-butyl-1-[[4-[(3-((1-carboxy-1,1-dimethylmethyloxy)phenyl)oxy]phenyl]methyl]-4-spirocyclopentyl-1H-imidazol-5(4H)-one;
2-butyl-1-[[4-[(2-((1-carboxy-1,1-dimethylmethyloxy)phenyl)methyl]phenyl]methyl]-4-spirocyclopentyl-1H-imidazol-5(4H)-one;
2-butyl-1-[(2′-((4-carboxy-4,4-dimethylbutan-1-yl)oxy)biphenyl-4-yl)methyl]4-spirocyclohexyl-1H-imidazol-5(4H)-one;
2-butyl-1-[(2′-((7-carboxy-7,7-dimethylheptan-1-yl)oxy)biphenyl-4-yl)methyl]-4-spirocyclopentyl-1H-imidazol-5(4H)-one;
2-butyl-1-[(2′-((4-carboxy-4,4-dimethylbutan-1-yl)oxy)biphenyl-4-yl)methyl]-4-spirocyclopentyl-1H-imidazol-5(4H)-one;
2-butyl-1-[[4-[(2-((1-carboxy-1,1-dimethylmethyloxy)phenyl)methyl]phenyl]methyl]-4-spirocyclohexyl-1H-imidazol-5(4H)-one;
2-butyl-1-[[4-[(3-((1-carboxy-1,1-dimethylmethyloxy)phenyl)thio]phenyl]methyl]4-spirocyclopentyl-1H-imidazol-5(4H)-one;
2-butyl-1-[[4-[(3-((1-carboxy-1,1-dimethylmethyloxy)phenyl)thio]phenyl]methyl]4-spirocyclohexyl-1H-imidazol-5(4H)-one;
2-butyl-1-[[4-[(4-((1-carboxy-1,1-dimethylmethyloxy)phenyl)thio]phenyl]methyl]4-spirocyclohexyl-1H-imidazol-5(4H)-one;
2-butyl-1-[(3′-((2-carboxy-2,2-dimethylethyl-1-yl)oxy)biphenyl-4-yl)methyl]-4-spirocyclopentyl-1H-imidazol-5(4H)-one;
2-butyl-1-[[4-[(4-((1-carboxy-1,1-dimethylmethyloxy)phenyl)methyl]phenyl]methyl]-4-spirocyclohexyl-1H-imidazol-5(4H)-one;
2-butyl-1-[[4-[(4-((1-carboxy-1,1-dimethylmethyloxy)phenyl)thio]phenyl]methyl]4-spirocyclopentyl-1H-imidazol-5(4H)-one;
2-butyl-1-[(3′-((1-carboxymethyl)oxy)biphenyl-4-yl)methyl]-4-spirocyclohexyl-1H-imidazol-5(4H)-one;
2-butyl-1-[(3′-((1-carboxy-1-methylmethyl)oxy)biphenyl-4-yl)methyl]-4-spirocyclopentyl-1H-imidazol-5(4H)-one;
2-butyl-1-[(3′-((1-carboxy-1-ethylmethyl)oxy)biphenyl-4-yl)methyl]-4-spirocyclohexyl-1H-imidazol-5(4H)-one;
2-butyl-1-[(3′-((1-carboxy-1-(1,1-dimethylmethyl)methyl)oxy)-biphenyl-4-yl)methyl]-4-spirocyclohexyl-1H-imidazol-5(4H)-one;
2-butyl-1-[(3′-((1-carbonylmethyl)oxy)-6′-fluoro-biphenyl-4-yl)methyl]-4-spirocyclohexyl-1H-imidazol-5(4H)-one;
2-butyl-1-[(3′-((1-carboxy-1-methylmethyl)oxy)-6′-fluoro-biphenyl-4-yl)methyl]-4-spirocyclohexyl-1H-imidazol-5(4H)-one;
2-butyl-1-[(3′-((1-carboxy-1-ethylmethyl)oxy)-6′-fluoro-biphenyl-4-yl)methyl]-4-spirocyclohexyl-1H-imidazol-5(4H)-one;
2-butyl-1-[(3′-((1-carboxy-1-(1,1-dimethylmethyl)-methyl)oxy)-6′-fluoro-biphenyl-4-yl)methyl]-4-spirocyclohexyl-1H-imidazol-5(4H)-one;
2-butyl-1-[(3-((1-carboxy-1,1-dimethylmethyl)oxy)-6′-fluoro-biphenyl-4-yl)methyl]-4-spirocyclohexyl-1H-imidazol-5(4H)-one;
2-butyl-4-spirocyclohexyl-1-[(3′-((1-(tetrazol-5-yl)methyl)oxy)-biphenyl-4-yl)methyl]-1H-imidazol-5(4H)-one;
2-butyl-1-[(6′-fluoro-3′-((1-(tetrazol-5-yl)methyl)oxy)biphenyl-4-yl)methyl]-4-spirocyclohexyl-1H-imidazol-5(4H)-one;
2-butyl-1-[(3′-((1-carboxy-1-(1,1-dimethylmethyl)methyl)oxy)-biphenyl-4-yl)methyl]-4,4-diethyl-1H-imidazol-5(4H)-one;
2-butyl-1-[(3′-((1-carboxy-1-ethylmethyl)oxy)biphenyl-4-yl)methyl]-4,4-diethyl-1H-imidazol-5(4H)one;
2-butyl-1-[(3′-((1-carboxy-1,1-dimethylmethyl)oxy)biphenyl-4-yl)methyl]-4,4-diethyl-1H-imidazol-5(4H)one;
2-butyl-1-[(3′-((1-carboxy-1-methylmethyl)oxy)biphenyl-4-yl)methyl]-4,4-diethyl-1H-imidazol-5(4H)one;
2-butyl-1-[(3′-((1-carboxy-1-spirocyclobutylmethyl)oxy)-biphenyl-4-yl)methyl]-4-spirocyclohexyl-1H-imidazol-5(4H)-one;
2-butyl-1-[(3′-((1-carboxy-1-(1,1-dimethylmethyl)methyl)oxy)-6′-fluoro-biphenyl-4-yl)methyl]-4,4-diethyl-1H-imidazol-5(4H)one;
2-butyl-1-[(3′-((1-carboxy-1,1-dimethylmethyl)oxy)-6′-fluoro-biphenyl-4-yl)methyl]-4,4-diethyl-1H-imidazol-5(4H)one;
2-butyl-1-[(3′-((1-carboxy-1-methylmethyl)oxy)-3-methyl-biphenyl-4-yl)methyl]4-spirocyclohexyl-1H-imidazol-5(4H)one;
2-butyl-1-[(3′-((1-carboxy-1-ethylmethyl)oxy)-3-methyl-biphenyl-4-yl)methyl]4-spirocyclohexyl-1H-imidazol-5(4H)one;
2-butyl-1-[(3′-((1-carboxy-1 1-dimethylmethyl)oxy)-3′-methyl-biphenyl-4-yl)methyl]-4-spirocyclohexyl-1H-imidazol-5(4H)-one;
2-butyl-1-[(3′-((1-carboxy-1-(1,1-dimethylmethyl)methyl)oxy)-3-methyl-biphenyl-4-yl)methyl]-4-spirocyclohexyl-1H-imidazol-5(4H)one;
2-butyl-1-[(3′-((1-carboxy-1,1-dimethylmethyl)oxy)biphenyl-3-yl)methyl]-4-spirocyclopentyl-1H-imidazol-5(4H)-one;
2-butyl-1-[(2′-((1-carboxy-1,1-dimethylmethyl)oxy)biphenyl-3-yl)methyl]-4-spirocyclopentyl-1H-imidazol-5(4H)-one;
2-butyl-1-[(3′-((1-carboxy-1,1-dimethylmethyl)oxy)-6′-propyl-biphenyl-4-yl)methyl]4-spirocyclopentyl-1H-imidazol-5(4H)-one;
2-butyl-1-[(4′-((1-carboxy-1,1-dimethylmethyl)oxy)biphenyl-3-yl)methyl]-4-spirocyclopentyl-1H-imidazol-5(4H)-one;
2-butyl-1-[(3′-((1-carboxy-1,1-dimethylmethyl)oxy)-2′-fluoro-biphenyl-4-yl)methyl]-4-spirocyclopethyl-1H-imidazol-5(4H)-one;
2-butyl-1-[(3′-((1-carboxy-1,1-dimethylmethyl)oxy)-4′-methoxy-biphenyl-4-yl)methyl]-4-spirocyclopethyl-1H-imidazol-5(4H)-one;
2-butyl-1-[(3′-((1-carboxy-1,1-dimethylmethyl)oxy)-6′-ethyl-biphenyl-4-yl)methyl]-4-spirocyclopentyl-1H-imidazol-5(4H)-one;
2-butyl-1-[(3′-((1-carboxy-1,1-dimethylmethyl)oxy)-4′-isobutyl-biphenyl-4-yl)methyl]-4-spirocyclopentyl-1H-imidazol-5(4H)-one;
2-butyl-1-[(3′-((1-carboxy-1,1-dimethylmethyl)oxy)-2-methoxy-biphenyl-4-yl)methyl]-4-spirocyclopethyl-1H-imidazol-5(4H)-one;
2-butyl-1-[(3′-((1-carboxy-1-methylmethyl)oxy)-6′-propyl-biphenyl-4-yl)methyl]4-spirocyclopentyl-1H-imidazol-5(4H)-one;
2-butyl-1-[(3′-((1-carboxymethyl)oxy)-6′-propyl-biphenyl-4-yl)methyl]4-spirocyclopentyl-1H-imidazol-5(4H)-one;
2-butyl-1-[(3′-((1-carboxy-1-ethylmethyl)oxy)-6′-propyl-biphenyl-4-yl)methyl]4-spirocyclopentyl-1H-imidazol-5(4H)-one;
2-butyl-1-[(3′-((1-carboxy-1-(1,1-dimethylmethyl)methyl)oxy)-6′-propyl-biphenyl-4-yl)methyl]-4-spirocyclopentyl-1H-imidazol-5(4H)-one;
2-butyl-1-[(3′-((1-carboxy-1,1-dimethylmethyl)oxy)-6′-isobutyl-biphenyl-4-yl)methyl]-4-spirocyclopentyl-1H-imidazol-5(4H)-one;
2-butyl-1-[(3′-((1-carboxy-1 1-dimethylmethyl)oxy)-3-ethyl-biphenyl-4-yl)methyl]-4-spirocyclopentyl-1H-imidazol-5(4H)-one;
2-butyl-1-[(3′-((1-carboxy-1,1-dimethylmethyl)oxy)-6′-cyano-biphenyl-4-yl)methyl]-4-spirocyclopentyl-1H-imidazol-5(4H)-one;
2-butyl-1-[(3′-((1-carboxy-1,1-dimethylmethyl)oxy)-3-methoxy-biphenyl-4-yl)methyl]-4-spirocyclopethyl-1H-imidazol-5(4H)-one;
2-butyl-1-[(3′-((1-carboxy-1,1-dimethylmethyl)oxy)-2-methyl-biphenyl-4-yl)methyl]-4-spirocyclopethyl-1H-imidazol-5(4H)-one;
2-butyl-1-[[2-[(4-(1-carboxy-1,1-dimethylmethyloxy)phenyl)-6-methyl-thiazolo[3,2-b][1,2,4]triazol-5-yl]methyl]-4-spirocyclopentyl-1H-imidazol-5(4H)-one;
2-butyl-1-[[2-[(3-(1-carboxy-1,1-dimethylmethyloxy)phenyl)-6-methyl-thiazolo[3,2-b][1,2,4]triazol-5-yl]methyl]-4-spirocyclopentyl-1H-imidazol-5(4H)-one;
2-butyl-1-[(3′-((1-carboxy-1,1-dimethylmethyl)oxy)-3-propyl-biphenyl-4-yl)methyl]4-spirocyclopentyl-1H-imidazol-5(4H)-one;
2-butyl-1-[(3′-((1-carboxy-1,1-dimethylmethyl)oxy)-4′-nitro-biphenyl-4-yl)methyl]-4-spirocyclopentyl-1H-imidazol-5(4H)-one;
2-butyl-1-[(3′-((1-carboxy-1,1-dimethylmethyl)oxy)-3-trifluoromethyl-biphenyl-4-yl)methyl]-4-spirocyclopethyl-1H-imidazol-5(4H)-one;
2-butyl-1-[(3′-((1-carboxy-1,1-dimethylmethyl)oxy)-3-nitro-biphenyl-4-yl)methyl]-4-spirocyclopentyl-1H-imidazol-5(4H)-one;
2-butyl-1-[(3′-((1-carboxy-1,1-dimethylmethyl)oxy)-4′-propyl-biphenyl-4-yl)methyl]4-spirocyclopentyl-1H-imidazol-5(4H)-one;
2-butyl-1-[(3′-((1-(tetrazol-5-yl)-1-ethylmethyl)oxy)-biphenyl-4-yl)methyl]-4-spirocyclopentyl-1H-imidazol-5(4H)-one;

25- Compounds according to claim 1 as medicines.

26- A pharmaceutical composition comprising, in a pharmaceutically acceptable support, at least one of the compounds as defined in claim 1, possibly in association with one or several other therapeutic and/or cosmetic active constituents.

27- A pharmaceutical composition comprising, in a pharmaceutically acceptable support, at least one of the compounds as defined in claim 1 in association with one or several of the compounds selected from the list below:
an anti-diabetic
insulin
a lipid-lowering and/or cholesterol-lowering molecule
an anti-hypertensive or hypotensive agent
an anti-platelet agent
an anti-obesity agent
an anti-inflammatory agent
an antioxidant agent
an agent used in the treatment of cardiac insufficiency
an agent used in the treatment of coronary insufficiency
an anti-cancer drug
an anti-asthma drug
a corticoid used in treating skin pathologies
a vasodilator and/or anti-ischemic agent.

28- The pharmaceutical composition according to claim 26, for the treatment of complications associated with metabolic syndrome, diabetes, dyslipidemias, atherosclerosis, cardiovascular diseases, obesity, hypertension, inflammatory diseases, insulin resistance, neurodegenerative pathologies, cancers, and/or to decrease the global cardiovascular risks.

29- The pharmaceutical composition according to claim 26 for treating dyslipidemias and/or hypertension.

30- Use of at least one compound such as defined in claim 1, for the preparation of a composition for the treatment of complications associated with metabolic syndrome, diabetes, dyslipidemias, atherosclerosis, cardiovascular diseases, obesity, hypertension, inflammatory diseases, insulin resistance, neurodegenerative pathologies, cancers, and/or to decrease the global cardiovascular risks.


SEQUENCE LISTING (18 sequences)

SEQ ID NO: 1 - DNA, 22 bp, artificial
  sense primer PDK4
tactccactgctccaacacctg

SEQ ID NO: 2 - DNA, 20 bp, artificial
  antisense primer PDK4
gttcttcggttccctgcttg

SEQ ID NO: 3 - DNA, 20 bp, artificial
  sense primer ACO
gaagccagcgttacgaggtg

SEQ ID NO: 4 - DNA, 20 bp, artificial
  antisense primer ACO
tggagttcttgggacgggtg

SEQ ID NO: 5 - DNA, 20 bp, artificial
  sense primer APO CIII
ctcttggctctcctggcatc

SEQ ID NO: 6 - DNA, 20 bp, artificial
  antisense primer APO CIII
gcatcctggaccgtcttgga

SEQ ID NO: 7 - DNA, 22 bp, artificial
  sense primer CPT1b
ggactgagactgtgcgttcctg

SEQ ID NO: 8 - DNA, 20 bp, artificial
  antisense primer CPT1b
agtgcttggcggatgtggtt

SEQ ID NO: 9 - DNA, 22 bp, artificial
  sense primer FGb
aagaagatggtggtggctggtg

SEQ ID NO: 10 - DNA, 20 bp, artificial
  antisense primer FGb
gggactattgctgtgggaag

SEQ ID NO: 11 - DNA, 20 bp, artificial
  sense primer PEPCK
aaggaaaacgccttgaacct

SEQ ID NO: 12 - DNA, 20 bp, artificial
  antisense primer PEPCK
gtaagggaggtcggtgttga

SEQ ID NO: 13 - DNA, 21 bp, artificial
  sense primer MCP1
agtcttcggagtttgggtttg

SEQ ID NO: 14 - DNA, 21 bp, artificial
  antisense primer MCP1
aggaagatctcagtgcagagg

SEQ ID NO: 15 - DNA, 25 bp, artificial
  sense primer 36B4
catgctcaacatctcccccttctcc

SEQ ID NO: 16 - DNA, 25 bp, artificial
  antisense primer 36B4
gggaaggtgtaatccgtctccacag

SEQ ID NO: 17 - DNA, 22 bp, artificial
  sense primer 18S
cggacacggacaggattgacag

SEQ ID NO: 18 - DNA, 20 bp, artificial
  antisense primer 18S
aatctcgggtggctgaacgc